(19) United States
(12) Patent Application Publication
(10) Pub. No.: US 2026/0367702 A1
(43) Pub. Date: Nov. 19, 2026

(54) Methods and Systems for Translation of Neural Activity into Embodied Digital-Avatar Animation

(71) Applicant: The Regents of the University of California, Oakland, CA (US)

(72) Inventors: Edward Chang, San Francisco, CA (US); Gopala Krishna Anumanchipalli, Oakland, CA (US); Kaylo Littlejohn, San Francisco, CA (US); Sean Metzger, San Francisco, CA (US)

(21) Appl. No.: 19/486,880

(22) PCT Filed: Jun. 6, 2024
(86) PCT No.: PCT/US2024/032886

§ 371 Date: Nov. 21, 2025

Related U.S. Application Data
(60) Provisional application No. 63/471,485, filed on Jun. 6, 2023.

(51) Int. Cl. G06F 3/01; A61B 5/00; A61B 5/37; A61B 5/374; G06N 3/0442; G06N 3/0455; G06N 3/0464; G06T 13/40; G10L 15/06; G10L 15/16; G10L 15/24

(52) U.S. Cl. CPC G06F 3/015; A61B 5/37; A61B 5/374; A61B 5/7267; G06N 3/0442; G06N 3/0455; G06N 3/0464; G06T 13/40; G10L 15/063; G10L 15/16; G10L 15/24

(57) ABSTRACT

Methods of assisting individuals with communication are provided. In the disclosed methods, cortical activity from a region of the brain associated with movement, speech production, and/or language perception is recorded while an individual attempts to perform an action (e.g., to say words, express an emotion, perform a movement, etc.). Deep learning computational models are used to detect and decode the attempted action from the recorded brain activity. Decoding of actions from brain activity may be aided by the use of self-supervised machine learning techniques, which discretize each action into one or more action representations that serve as an effective intermediary for decoding neural activity patterns and features into meaningful action outputs. Methods for synthesizing decoded actions into audio and/or visual stimuli are also provided, allowing for more naturalistic and expressive communication for individuals who are unable to speak or experience other mobility limitations that inhibit full embodied communication.

### CROSS-REFERENCE TO RELATED APPLICATION

Pursuant to 35 U.S.C. § 119(e), this application claims priority to the filing date of U.S. Provisional Patent Application Ser. No. 63/471,485 filed Jun. 6, 2023, the disclosure of which is herein incorporated by reference in its entirety.

### STATEMENT REGARDING FEDERALLY SPONSORED RESEARCH

This invention was made with government support under grant no. NIH U01 DC018671-01A1, awarded by the National Institutes of Health (NIH). The government has certain rights in the invention.

### INTRODUCTION

Speech is the ability to express thoughts and ideas through spoken words. Anarthria, or the loss of the ability to articulate speech, can result from a variety of conditions, including stroke, traumatic brain injury, and amyotrophic lateral sclerosis. For paralyzed individuals with severe movement impairment, anarthria hinders communication with family, friends, and caregivers, reducing self-reported quality of life. Speech neuroprostheses have the potential to restore communication to people living with paralysis, but naturalistic speed and expressivity remain elusive. Previous demonstrations have shown that it is possible to decode speech from the brain activity of a person with paralysis, but only in the form of text and with limited speed and vocabulary. While text outputs are good for basic communication and messaging, speaking has rich prosody, expressiveness, and identity that can enhance embodied communication beyond what can be conveyed in text alone.

### SUMMARY

Thus, there remains a need for better methods and systems for restoring the ability to communicate to patients with anarthria and paralysis. This invention provides such new and useful methods and systems, addressing the limitations mentioned above. To accomplish this, the invention leverages recent advances in neural interfaces and machine learning techniques which enable models to be trained from highly stable neural recordings using only task go cues for data segmentation, i.e., without any other alignment of neural activity and output features. The methods and systems of the invention, e.g., as described in greater detail below, find use in assisting individuals with communication. In particular, methods, devices, and systems are provided that facilitate full, embodied communication to people living with severe paralysis by restoring the ability to produce speech sounds and facial movements related to speaking, as well as by restoring the ability to perform non-speech communicative gestures.

Methods of assisting individuals with communication are provided. In the disclosed methods, cortical activity from a region of the brain associated with movement, speech production, and/or language perception is recorded while an individual attempts to perform an action (e.g., to say words, express an emotion, perform a movement, etc.). Deep learning computational models are used to detect and decode the attempted action from the recorded brain activity. Decoding of actions from brain activity is aided by the use of self-supervised machine learning techniques, which discretize each action into one or more action representations that serve as an effective intermediary for decoding neural activity patterns and features into meaningful action outputs. In addition, methods for synthesizing decoded actions into audio and/or visual stimuli are provided, allowing for more naturalistic and expressive communication for individuals who are unable to speak or experience other mobility limitations that inhibit full embodied communication.

In one aspect, methods of producing speech audio directly from the neural activity of an individual are provided. Aspects of the methods include: positioning a neural recording device comprising an electrode at a location in a sensorimotor cortex region of the brain of the subject to record brain electrical signal data associated with attempted speech by the subject; positioning an interface in communication with a computing device at a location on the head of the subject, wherein the interface is connected to the neural recording device; recording the brain electrical signal data associated with attempted speech by the subject using the neural recording device, wherein the interface receives the brain electrical signal data from the neural recording device and transmits the brain electrical signal data to a processor of the computing device; and decoding one or more speech sounds from the recorded brain electrical signal data using the processor, wherein the processor is programmed to use a machine learning model for the decoding.

In certain embodiments, the subject has difficulty speaking because of anarthria, a stroke, a traumatic brain injury, a brain tumor, or amyotrophic lateral sclerosis. In some embodiments, the subject has a speech intelligibility of 10% or less for prompted words. In some embodiments, the subject is paralyzed.

In certain embodiments, neural activity from the sensorimotor cortex region of the subject's brain is recorded using a neural recording device comprising an electrode. In some embodiments, the neural recording device includes an electrocorticography (ECoG) electrode array, such as a high density ECoG electrode array. For example, the high density ECoG electrode array may include 250 electrodes or more. In some embodiments, the electrodes may be non-penetrating surface electrodes.

In certain embodiments, the neural recording device is positioned on the pial surface of the sensorimotor cortex such that the device covers a middle portion of the superior and/or middle temporal gyrus, the precentral gyrus, and/or the postcentral gyrus. In some embodiments, the neural recording device is implanted over the subject's lateral cortex and is centered on the central sulcus. In some embodiments, the neural recording device acquires brain electrical signal data from a sensorimotor cortex region selected from the precentral gyrus, postcentral gyrus, superior temporal gyrus, middle temporal gyrus, or any combination thereof.

In certain embodiments, neural signals acquired by the neural recording device are processed to extract high-gamma activity (HGA) and/or low-frequency signals (LFS). In some embodiments, the HGA electrical signal data may include neural oscillations in a range from 70 Hz to 150 Hz. In some embodiments, the LFS electrical signal data may include neural oscillations in a range from 0.3 Hz to 17 Hz.

In certain embodiments, the one or more speech sounds form a word or a sentence. In some embodiments, the subject is limited to a specified word set for the attempted speech. In some embodiments, the word set is chosen in order to enable the subject to express basic concepts and communicate caregiving needs. In some embodiments, the subject may switch between two or more word sets depending on context. For example, the subject may select a first word set to communicate with a caregiver and a second word set to discuss a baseball game with a friend or family member. In some embodiments, the word set includes 100 words or more, or 350 words or more, or 1000 words or more.

In certain embodiments, the method further includes providing a series of go cues to the subject indicating when the subject should initiate attempted speech. In some embodiments, the series of go cues are provided visually on a display. In some embodiments, each go cue is preceded by a countdown to the presentation of the go cue, wherein the countdown for the next spoken word or sentence is provided visually on the display and automatically started after each go cue. In some embodiments, the series of go cues are provided with a set interval of time between each go cue. In some embodiments, the subject can control the set interval of time between each go cue. In some embodiments, the processor is programmed to use the recorded brain electrical signal data within a time window following the go cue.

In certain embodiments, the machine learning model used to decode the one or more speech sounds from the recorded brain electrical signal data includes a neural network. In some embodiments, the neural network includes one or more convolutional layers and is bidirectional. In some embodiments, the neural network is a recurrent neural network (RNN) such as an RNN including gated recurrent units (GRUs).

In certain embodiments, brain electrical signal data is decoded into one or more speech sounds using intermediate representations. In some embodiments, the intermediate representations are a set of discrete speech units. In some embodiments, the discrete speech units are derived using an encoding machine learning model, such as an encoding machine learning model including a neural network. In some embodiments, the neural network includes a transformer encoder. For example, the encoding machine learning model may be a Hidden-Unit Bidirectional Encoder Representations from Transformers (HuBERT) model. In some embodiments, the discrete speech units are derived during self-supervised training of the machine learning model.

In certain embodiments, the methods of producing speech audio directly from neural activity further includes training the machine learning model used to decode the one or more speech sounds from the recorded brain electrical signal data. In some embodiments, the training includes: obtaining reference electronic speech waveforms for a plurality of phrases; encoding each reference speech waveform into a temporal sequence of discrete speech units using the encoding model; recording the brain electrical signal data associated with attempted speech by the subject for each one of the plurality of phrases; training the decoding machine learning model to predict the most likely discrete speech unit associated with a segment of electrical signal data using the speech units derived from the reference speech waveforms and the brain electrical signal data associated with attempted speech for each one of the plurality of phrases.

In certain embodiments, the reference electronic speech waveforms are obtained from a recruited speaker. In some embodiments, the reference electronic speech waveforms are obtained using a text-to-speech algorithm.

In certain embodiments, the decoding machine learning model is trained to learn mappings between neural activity patterns of electrical signals in the brain electrical signal data and the discrete speech units. In certain embodiments, the decoding machine learning model is trained using a CTC loss function. In some embodiments, the training may further include validating the machine learning model or machine learning models. In some embodiments, the methods may further include testing the trained machine learning model.

In certain embodiments, each speech sound is decoded from one or more discrete speech units using a speech synthesizer. In some embodiments, the speech synthesizer includes a machine learning model. In some embodiments, the synthesizing machine learning model is configured to generate a Mel spectrogram from one or more discrete speech units. In some embodiments, the speech synthesizer further includes a vocoder configured to synthesize an electronic speech waveform from the spectrogram.

In certain embodiments, the electronic speech waveform decoded from recorded brain electrical signal data and synthesized using the speech synthesizer is transformed into a personalized electronic speech waveform. For example, the electronic speech waveform may be transformed such that it resembles the subject's own voice. In some embodiments, the electronic speech waveform is transformed using a machine learning model, such as the YourTTS zero-shot voice conversion model.

In certain embodiments, the electronic speech waveform decoded from recorded brain electrical signal data and synthesized using the speech synthesizer is converted into an audible speech waveform using a loudspeaker. In some embodiments, an electronic output device or system is controlled using the electronic speech waveform. For example, an animated avatar may be controlled to perform orofacial movements based on the electronic speech waveform.

In another aspect, the methods of producing speech audio directly from neural activity are provided as computer implemented methods. Aspects of the computer implemented methods include: receiving the brain electrical signal data associated with attempted speech by the subject using the neural recording device; and decoding one or more speech sounds from the recorded brain electrical signal data using a machine learning model. In some embodiments, the computer in implemented methods further include implementing any of the embodiments of the methods of producing speech audio directly from recorded brain electrical signals described herein using a computer.

In certain embodiments, the computer implemented method further includes storing a user profile for the subject including information regarding the patterns of electrical signals in the recorded brain electrical signal data associated with attempted speech by the subject.

In another aspect, a non-transitory computer-readable medium is provided, the non-transitory computer-readable medium including program instructions that, when executed by a processor in a computer, causes the processor to perform the computer implemented methods described herein.

In another aspect, a kit is provided, the kit including the non-transitory computer-readable medium and instructions for decoding brain electrical signal data associated with attempted speech by a subject.

In another aspect, a system for producing speech audio directly from neural activity is provided, the system including: a neural recording device including an electrode adapted for positioning at a location in a sensorimotor cortex region of the brain of the subject to record brain electrical signal data associated with attempted speech by the subject; a processor programmed to use a machine learning model to decode one or more speech sounds from the recorded brain electrical signal data according to a computer implemented method described herein; an interface in communication with a computing device, said interface adapted for positioning at a location on the head of the subject, wherein the interface receives the brain electrical signal data from the neural recording device and transmits the brain electrical signal data to the processor; and an audio speaker for playing the one or more speech sounds from the recorded brain electrical signal data.

In certain embodiments, the neural recording device includes an ECoG electrode array, such as a high density ECoG electrode array. For example, the high density ECoG electrode array may include 250 electrodes or more. In some embodiments, the electrodes may be non-penetrating surface electrodes.

In certain embodiments, the interface includes a percutaneous pedestal connector attached to the subject's cranium.

In certain embodiments, the interface further includes a headstage that is connectable to the percutaneous pedestal connector.

In certain embodiments, the processor is provided by a computer or handheld device (e.g., a cell phone or tablet).

In another aspect, a kit including a system described herein and instructions for using the system for recording and decoding brain electrical signal data associated with an attempted action by a subject.

In one aspect, methods of controlling an electronic output device or system to perform one or more actions using brain electrical signals are provided. Aspects of the methods include: positioning a neural recording device including an electrode at a location in a sensorimotor cortex region of the brain of the subject to record brain electrical signal data associated with an attempted action by the subject; positioning an interface in communication with a computing device at a location on the head of the subject, wherein the interface is connected to the neural recording device; recording the brain electrical signal data associated with the attempted action by the subject using the neural recording device, wherein the interface receives the brain electrical signal data from the neural recording device and transmits the brain electrical signal data to a processor of the computing device; decoding one or more electronic output device or system actions from the recorded brain electrical signal data, wherein the processor is programmed to use a machine learning model for the decoding; and controlling the electronic output device or system to perform the one or more decoded electronic output device or system actions.

In certain embodiments, the subject has difficulty speaking because of anarthria, a stroke, a traumatic brain injury, a brain tumor, or amyotrophic lateral sclerosis. In some embodiments, the subject is paralyzed. In some embodiments, the subject is quadriplegic and/or experiences partial or total facial paralysis.

In certain embodiments, neural activity from the sensorimotor cortex region of the subject's brain is recorded using a neural recording device including an electrode. In some embodiments, the neural recording device includes an ECoG electrode array, such as a high density ECoG electrode array. For example, the high density ECoG electrode array may include 250 electrodes or more. In some embodiments, the electrodes may be non-penetrating surface electrodes.

In certain embodiments, the neural recording device is positioned on the pial surface of the sensorimotor cortex such that the device covers a middle portion of the superior and/or middle temporal gyrus, the precentral gyrus, and/or the postcentral gyrus. In some embodiments, the neural recording device is implanted over the subject's lateral cortex and is centered on the central sulcus. In some embodiments, the neural recording device acquires brain electrical signal data from a sensorimotor cortex region selected from the precentral gyrus, postcentral gyrus, superior temporal gyrus, middle temporal gyrus, or any combination thereof.

In certain embodiments, neural signals acquired by the neural recording device are processed to extract HGA and/or LFS. In some embodiments, the HGA electrical signal data may include neural oscillations in a range from 70 Hz to 150 Hz. In some embodiments, the LFS electrical signal data may include neural oscillations in a range from 0.3 Hz to 17 Hz.

In certain embodiments, the interface includes a percutaneous pedestal connector attached to the subject's cranium.

In certain embodiments, the interface further includes a headstage that is connectable to the percutaneous pedestal connector.

In certain embodiments, the processor is provided by a computer or handheld device (e.g., a cell phone or tablet).

In certain embodiments, the attempted action performed by the subject is the movement of one or more body parts of the subject. In some embodiments, the one or more body parts is one or more limbs of the subject, such as the subject's arms or fingers. In some embodiments, the movement of the one or more body parts is directed to perform a task. In some embodiments, the attempted action performed by the subject is attempted speech.

In certain embodiments, the methods further include mapping the actions an electronic output device or system is capable of performing to attempted actions performed by the subject. In some embodiments, the attempted action performed by the subject is different than the one or more electronic device or system actions. In some embodiments, the attempted action performed by the subject is a hand gesture and the action of the electronic output device or system is powering on/off or opening/closing. In some embodiments, the attempted action performed by the subject corresponds to the one or more electronic device or system actions.

In certain embodiments, the subject is limited to a specified action set for the attempted action. In some embodiments, the action set is chosen in order to enable the subject to control the electronic output device or system for a specific purpose. In some embodiments, the subject may switch between two or more action sets depending on context. For example, the electronic device or system may include a humanoid avatar and the subject may select a first action set to control the avatar for the purpose of communicating with a caregiver and a second action set to control the avatar for the purpose of playing a baseball video game. In some embodiments, the action set includes 100 actions or more, or 350 actions or more, or 1000 actions or more.

In certain embodiments, the method further includes providing a series of go cues to the subject indicating when the subject should initiate an attempted action. In some embodiments, the series of go cues are provided visually on a display. In some embodiments, each go cue is preceded by a countdown to the presentation of the go cue, wherein the countdown for the action is provided visually on the display and automatically started after each go cue. In some embodiments, the series of go cues are provided with a set interval of time between each go cue. In some embodiments, the subject can control the set interval of time between each go cue. In some embodiments, the processor is programmed to use the recorded brain electrical signal data within a time window following the go cue.

In certain embodiments, the machine learning model used to decode the one or more electronic output device or system actions from the recorded brain electrical signal data includes a neural network. In some embodiments, the neural network includes one or more convolutional layers and is bidirectional. In some embodiments, the neural network is an RNN such as an RNN including GRUs.

In certain embodiments, brain electrical signal data is decoded into one or more device or system actions using intermediate representations. In some embodiments, the intermediate representations are a set of discrete action representations. In some embodiments, the discrete action representations are derived using an encoding machine learning model, such as an encoding machine learning model including a neural network. In some embodiments, the neural network includes one or more components of a variational autoencoder and/or may include one or more rectified linear unit (ReLU) activations. For example, the encoding machine learning model may be a vector-quantized variational autoencoder (VQ-VAE) model. In some embodiments, the discrete action representations are derived during self-supervised training of the machine learning model.

In certain embodiments, the electronic output device or system is configured to produce or display the same action that is attempted by the subject. In some embodiments, the electronic device or system is a prosthetic limb. In some embodiments, the electronic device or system includes a humanoid avatar. For example, the electronic output device or system may include a visual display and/or a loudspeaker configured to present a humanoid avatar.

In certain embodiments, the methods of controlling an electronic output device or system to perform one or more actions using recorded neural activity further includes training the machine learning model used to decode the attempted action from the recorded brain electrical signal data. In some embodiments, the training includes: obtaining reference output device or system actions for a plurality of actions; encoding each reference action into a temporal sequence of discrete action representations using the encoding machine learning model; recording the brain electrical signal data associated with attempted action by the subject for each one of the plurality of actions; training the decoding machine learning model to predict the most likely discrete action representation associated with a segment of electrical signal data using the action representations derived from the reference output device or system actions and the brain electrical signal data associated with the attempted action for each one of the plurality of actions. In some embodiments, device or system actions include avatar animations. In some embodiments, the avatar animations are of the avatar performing the same action as the action attempted by the subject.

In certain embodiments, the reference avatar animations are obtained from an avatar-animation system. In some embodiments, the reference avatar animations are of the avatar performing orofacial movements for speech. In some embodiments, the speech orofacial movements include one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction.

In certain embodiments, the reference avatar animations are of the avatar performing non-speech communicative gestures. In some embodiments, the non-speech communicative gestures include the abduction, adduction, flexion, extension, and/or circumduction of one or more body parts. In some embodiments, the non-speech communicative gestures include emotional expressions using facial muscles. For example, the emotional expressions may include happy, sad, and/or surprised expressions.

In certain embodiments, the decoding machine learning model is trained to learn mappings between neural activity patterns of electrical signals in the brain electrical signal data and the discrete action representations. In certain embodiments, the decoding machine learning model is trained using a CTC loss function. In some embodiments, the training may further include validating the machine learning model or machine learning models. In some embodiments, the methods may further include testing the trained machine learning model.

In certain embodiments, each electronic output device or system action is decoded from one or more discrete action representations using a decoder. In some embodiments, the decoder includes a machine learning model. In some embodiments, the decoder machine learning model is trained with the encoding machine learning model or using the encoding machine learning model. In some embodiments, the encoding machine learning model and the decoder machine learning model are separate components of the same machine learning model. For example, a VQ-VAE may be used to discretize or encode each reference electronic output device or system action into one or more discrete action representations and the VQ-VAE's decoder may be used to decode electronic output device or system actions from discrete action representations decoded from recorded brain electrical signal data.

In certain embodiments, the attempted actions performed by the subject and the actions of the device or system include both speech associated actions and non-speech communicative gestures. In some embodiments, the decoding machine learning model is trained to discriminate between actions performed by different regions of the body and/or between speech associated actions and non-speech communicative gestures.

In certain embodiments, the electronic output device or system is controlled using actions decoded from multiple machine learning models. In some embodiments, the actions decoded from the multiple machine learning models occur concurrently and the electronic output device or system is controlled to perform the actions simultaneously. In some embodiments, separate decoding machine learning models are trained for speech and orofacial movements for speech.

In some embodiments, a decoded non-speech communicative gesture action affects how the electronic output device or system performs a decoded speech associated action. For example, a decoded emotional expression may affect the inflection of decoded speech performed by an avatar.

In certain embodiments, the electronic output device or system action decoded from recorded brain electrical signal data is used by the subject to communicate with one or more individuals in person. In some embodiments, the electronic device or system action decoded from recorded brain electrical signal data is used by the subject to communicate with one or more individuals virtually. For example, an animated avatar may be controlled to communicate with one or more individuals in an interactive virtual environment.

In certain embodiments, an animated avatar controlled using brain electrical signals is used by the subject to play a video game. In some embodiments, the avatar is used by the subject for physical therapy. In some embodiments, avatar is used by the subject to control one or more electronic devices in the subject's environment.

In another aspect, the methods of controlling an electronic output device or system to perform one or more actions using brain electrical signals are provided as computer implemented methods. Aspects of the computer implemented methods include: receiving the brain electrical signal data associated with an attempted action by the subject using the neural recording device; decoding one or more electronic output device or system actions from the recorded brain electrical signal data using a machine learning model; and controlling the electronic output device or system to perform the one or more decoded electronic output device or system actions. In some embodiments, the computer implemented methods further include implementing any of the embodiments of the methods of controlling an electronic output device or system using brain electrical signals described herein using a computer.

In certain embodiments, the computer implemented method further includes storing a user profile for the subject including information regarding the patterns of electrical signals in the recorded brain electrical signal data associated with an attempted action by the subject.

In another aspect, a non-transitory computer-readable medium is provided, the non-transitory computer-readable medium including program instructions that, when executed by a processor in a computer, causes the processor to perform the computer implemented methods described herein.

In another aspect, a kit is provided, the kit including the non-transitory computer-readable medium and instructions for decoding brain electrical signal data associated with an attempted action by a subject.

In another aspect, a system for controlling an avatar to perform one or more actions using brain electrical signals is provided, the system including: a neural recording device including an electrode adapted for positioning at a location in a sensorimotor cortex region of the brain of the subject to record brain electrical signal data associated with an attempted action by the subject; a processor programmed to use a machine learning model to decode an avatar animation from the recorded brain electrical signal data according to a computer implemented method described herein; an interface in communication with a computing device, said interface adapted for positioning at a location on the head of the subject, wherein the interface receives the brain electrical signal data from the neural recording device and transmits the brain electrical signal data to the processor; and a display component for displaying the avatar animation from the recorded brain electrical signal data.

In certain embodiments, the neural recording device includes an ECoG electrode array, such as a high density ECoG electrode array. For example, the high density ECoG electrode array may include 250 electrodes or more. In some embodiments, the electrodes may be non-penetrating surface electrodes.

In certain embodiments, the interface includes a percutaneous pedestal connector attached to the subject's cranium.

In certain embodiments, the interface further includes a headstage that is connectable to the percutaneous pedestal connector.

In certain embodiments, the processor is provided by a computer or handheld device (e.g., a cell phone or tablet).

In certain embodiments, the display component includes a computer monitor, a television, and/or a visual projection device. In some embodiments, the visual display includes a virtual reality headset, goggles, or contacts. In some embodiments, the visual display includes an augmented reality headset, goggles, or contacts.

In another aspect, a kit including a system described herein and instructions for using the system for recording and decoding brain electrical signal data associated with an attempted action by a subject.

### BRIEF DESCRIPTION OF THE FIGURES

FIG. 1 illustrates an overview of a multimodal speech decoding pipeline in a participant with vocal-tract paralysis in accordance with an embodiment of the invention.

FIGS. 2A to 2C depict multimodal speech decoding in a participant with vocal-tract paralysis in accordance with an embodiment of the invention. (A) a sagittal MRI showing brainstem atrophy (in the bilateral pons; red arrow) resulting from stroke. (B) an MRI reconstruction of the participant's brain overlaid with the locations of implanted electrodes. (C) an example of ECoG features resulting from attempted orofacial movements.

FIGS. 3A to 3E depict high-performance text decoding from neural activity in accordance with an embodiment of the invention. (A) is a schematic diagram of the text-decoding algorithm. (B) depicts median phone error rates with the 1024-word-General sentence set. (C) depicts word error rates for chance and real-time results. (D) depicts character error rates for chance and real-time results.

FIGS. 4A to 4B depict results of high-performance text decoding from neural activity in accordance with an embodiment of the invention. (A) depicts offline evaluation of error rates as a function of number of recording days and data quantity. (B) depicts real-time classification accuracy during attempts to silently say 26 NATO code words across many recording days.

FIG. 5 is a schematic diagram of the speech-synthesis decoding algorithm in accordance with an embodiment of the invention.

FIG. 6 depicts intelligible speech synthesis from neural activity in accordance with an embodiment of the invention. The top provides three example decoded spectrograms and waveforms from the 529-phrase-AAC sentence set. The bottom provides the corresponding reference spectrograms and waveforms representing the decoding targets.

FIGS. 7A to 7C depict results of intelligible speech synthesis from neural activity in accordance with an embodiment of the invention. (A) depicts Mel-cepstral distortions for the decoded waveforms. (B) shows perceptual word error rates from untrained human evaluators via a transcription task. (C) shows perceptual character error rates from the same human-evaluation results as (B).

FIG. 8 is a schematic diagram of the avatar decoding algorithm in accordance with an embodiment of the invention.

FIGS. 9A to 9B depict results of direct decoding of orofacial articulatory gestures from neural activity to drive an avatar in accordance with an embodiment of the invention. (A) binary perceptual accuracies from human evaluators on avatar animations generated from neural activity. (B) correlations for jaw, lip, and mouth-width movements between decoded avatar renderings and videos of real human speakers on the 1024-word-General sentence set.

FIGS. 10A to 10B depict results of direct decoding of orofacial articulatory gestures from neural activity to drive an avatar in accordance with an embodiment of the invention. (A) top: snapshots of avatar animations of 6 non-speech articulatory movements in the articulatory-movement task; bottom: confusion matrix depicting classification accuracy across the movements. (B) top: snapshots of avatar animations of 3 non-speech emotional expressions in the emotional-expression task; bottom: confusion matrix depicting classification accuracy across 3 intensity levels (high, medium, and low) of the 3 expressions, ordered via hierarchical agglomerative clustering on the confusion values.

FIGS. 11A to 11B depict articulatory encodings driving speech decoding. (A) is a mid-sagittal schematic of the vocal tract with phone place of articulation (POA) features labeled. (B) bottom-right: visualization of the locations of electrodes with the greatest encoding weights for labial, front-tongue, and vocalic phones on the electrocorticography array, the electrodes that most strongly encoded finger flexion during the NATO-motor task are also included.

FIGS. 12A to 12D depict articulatory encodings driving speech decoding. (A) illustrates phone-encoding vectors for each electrode computed by a temporal receptive-field model on neural activity recorded during attempts to silently say sentences from the 1024-word-General set, organized by unsupervised hierarchical clustering. (B) provides Z-scored POA encodings for each electrode, computed by averaging across positive phone encodings within each POA category. (C) and (D) provide projection of consonant (C) and vowel (D) phone encodings into a two-dimensional space via multidimensional scaling (MDS).

FIGS. 13A to 13C provide electrode-tuning comparisons between front-tongue phone encoding and tongue-raising attempts (A), labial phone encoding and lip-puckering attempts (B), and tongue-raising and lip-rounding attempts (C).

FIGS. 14A to 14C depict effects of anatomical coverage, electrode density, and feature sets on decoding performance. (A) depicts MRI reconstruction of the participant's brain overlaid with the locations of implanted electrodes. (B) and (C) provide the effect of excluding each region during training and testing on text-decoding word error rates (B) and NATO code-word classification accuracies (C).

FIGS. 15A to 15C depict effects of anatomical coverage, electrode density, and feature sets on decoding performance. (A) is a visualization of the checkerboard-downsampling procedure used to simulate a low-density electrocorticography array with 127 electrodes instead of 253. (B) and (C) provide the effect of modulating electrode density and feature set on text-decoding word error rates (B) and NATO code-word classification accuracies (C).

FIG. 16 provides results of simulated text decoding with a larger vocabulary in accordance with an embodiment of the invention. Text decoding results were simulated using a 42,391-word vocabulary on the blocks used for real-time evaluation with the 1024-word-General set.

FIG. 17 provides results of simulated text decoding on the 50-phrase-AAC sentence set in accordance with an embodiment of the invention. Text decoding results were simulated on the real-time blocks used for evaluation with the synthesis models.

FIG. 18 provides results of simulated text decoding on the 529-phrase-AAC sentence set in accordance with an embodiment of the invention. Text decoding results were simulated on the real-time blocks used for evaluation of the 529-phrase-AAC sentence set with the synthesis models.

FIG. 19 provides Mel-cepstral distortions using a personalized voice tailored to the participant in accordance with an embodiment of the invention. The Mel-cepstral distortion was calculated between decoded speech with the participant's personalized voice and reference waveforms for the 529-phrase-AAC, 50-phrase-AAC, and 1024-word-General set.

FIG. 20 provides examples of directly decoded articulatory gestures (colored) compared with reference articulatory gestures (black) in accordance with an embodiment of the invention. Examples were taken from the 50-phrase-AAC sentence set.

FIG. 21 provides correlations of directly decoded avatar articulatory gestures with reference articulatory gestures in accordance with an embodiment of the invention.

FIG. 22 provides correlations between avatar articulatory gestures with reference articulatory gestures using the acoustic approach in accordance with an embodiment of the invention.

FIG. 23 illustrates binary perceptual accuracy from human evaluation of silent videos extracted from the audio-visual synthesis task in accordance with an embodiment of the invention.

FIG. 24 provides correlations of facial landmark trajectories within healthy speakers and between healthy speakers and the avatar using the acoustic approach for avatar decoding the 1024-word-General sentence set or in accordance with an embodiment of the invention.

FIG. 25 provides classification results of emotional expressions. Full 15-fold cross validation classification accuracy for emotional expressions across different subsets of intensities in the emotional-expression task are depicted.

FIG. 26 illustrates cross-phone place-of-articulation encoding. For each electrode included in FIG. 11B the relationship between encoding of phone place of articulation (POA) categories was visualized.

FIGS. 27A to 27D demonstrate spatial distribution of electrode tuning to articulatory features. Shown are normalized [0-1] encoding weights across electrodes for (A) hand finger flexion, (B) labial phones, (C) front tongue phones, and (D) vocalic phones.

FIGS. 28A to 28B demonstrate that attempted finger flexion and speech are largely encoded orthogonally. (A) for each electrode, the normalized [0,1] encoding in response to attempted production of NATO code-words is plotted against attempted finger flexion in the NATO-motor task. (B) confusion matrix from the NATO-motor task, showing minimal confusion between hand and speech targets.

FIG. 29 illustrates a virtual environment for avatar decoding in accordance with an embodiment of the invention.

FIGS. 30A to 30C provide examples of dlib facial-landmark detection in accordance with an embodiment of the invention. (A) an example of detected facial landmarks overlaid on a frame selected from a video rendering of an avatar during a single trial and using the direct approach to avatar decoding. (B) an example set of plotted detected facial landmark key points from a video rendering of an avatar during a single trial and using the direct approach to avatar decoding. (C) an example set of plotted facial landmark key points that shows exemplar facial landmark detection of a human face.

FIG. 31 depicts the distribution of phone-encoding r-values across electrodes. Shown are encoding r-values across electrodes from the linear encoding model trained to predict each electrode's high gamma activity from phoneme emission probabilities in accordance with an embodiment of the invention.

FIG. 32 provides a flow diagram depicting a method of controlling a fully embodied virtual avatar using recorded brain electrical signal data in accordance with an embodiment of the invention.

FIG. 33 provides a flow diagram depicting a method of controlling and displaying a fully embodied virtual avatar using recorded brain electrical signal data in accordance with an embodiment of the invention.

FIG. 34 provides a flow diagram depicting a method for training a machine learning model to predict avatar movements using recorded neural activity in accordance with an embodiment of the invention.

FIG. 35 illustrates an overview of a non-speech communicative gesture neural-decoding pipeline in accordance with embodiments of the invention.

FIG. 36 provides a block diagram of a pipeline for concurrent multi-effector neural-decoding in accordance with embodiments of the invention.

FIG. 37 provides a block diagram of a pipeline for non-verbal linguistic neural-decoding in accordance with embodiments of the invention.

FIGS. 38A to 38B depict an overview of a naturalistic streaming silent-speech neuroprosthesis in accordance with an embodiment of the invention. (A) an overview of the streaming speech-synthesis and text-decoding pipeline. (B) an example of an online waveform (top) and spectrogram (bottom) from the 1024-word-General set.

FIGS. 39A to 39F provide results of online continuously streaming synchronized speech synthesis and text decoding from neural activity in accordance with an embodiment of the invention. (A) latency for speech-synthesis and text-decoding. (B) synchronization time between the speech-synthesis onset and text-decoding onset. (C) synthesized words per minute compared to delayed synthesis. (D) phoneme error rates. (E) word error rates. (F) character error rates.

FIGS. 40A to 40E illustrate offline long-form continuous speech decoding with implicit speech detection. (A) Top: a heatmap of log-scaled high-gamma activity (HGA) from the top 20 most speech-responsive electrodes during silent speech attempts of an entire block (5.9 minutes) of 1024-word-General sentences. Bottom: a continuously synthesized speech waveform from the aforementioned neural activity. (B) latency between the detected onset of silently attempted speech to synthesized speech output onset and latency between the detected offset of silently attempted speech to synthesized speech output offset. (C) phoneme error rates. (D) word error rates. (E) character error rates.

FIGS. 41A to 41C illustrate speech synthesis generalization across silent-speech interfaces in accordance with an embodiment of the invention. (A) phoneme error rates. (B) word error rates. (C) character error rates.

FIGS. 42A to 42D demonstrate model-generated auditory feedback does not interfere with articulatory-driven speech decoding. (A) placement of the electrodes on the speech sensorimotor cortex. (B) contribution maps calculated from two conditions: blocks with auditory feedback during online speech-synthesis demonstrations (left) and blocks without decoder feedback (right). (C) contribution comparison for each channel, colored by anatomical region. (D) for both speech and text, there is no significant difference in decoding performance between conditions.

FIG. 43A to 43C provides decoding accuracy results for real-time text-to-speech decoding using the 1024-word-General sentence set in accordance with an embodiment of the invention.

FIG. 44 provides latency results from go-cue to speech-decoding in accordance with an embodiment of the invention.

FIGS. 45A to 45C provide results characterizing the latency of the speech synthesis and text decoding system in accordance with an embodiment of the invention. (A) latency per time step for the neural encoder, speech joiner and beam search, speech synthesizer, text joiner and beam search, and complete system. (B) latency by module. (C) success rate averaged across time for all trials.

FIGS. 46A to 46C provide region-exclusion analysis for 1024-word-General decoding in accordance with an embodiment of the invention. (A) phoneme error rates. (B) word error rates. (C) character error rates.

FIGS. 47A to 47C provide decoding accuracy results by the length of training data for models in accordance with embodiments of the invention. (A) phoneme error rates. (B) word error rates. (C) character error rates.

FIGS. 48A to 48C provide real-time decoding performance of speech synthesis using predicted transcripts gathered from perceptual evaluations or automatic speech recognition using in accordance with embodiments of the invention. (A) phoneme error rates. (B) word error rates. (C) character error rates.

FIGS. 49A to 49L demonstrate reading and listening do not interfere with speech decoding using methods in accordance with embodiments of the invention. (A) chronically implanted ECoG grids in two participants. (B) a speech decoding system. (C) the decoding framework. (D) the false positive rate (FPR) of the full speech decoding system. (E) the true positive rate (TPR) of the full speech decoding system. (F) electrode contributions for the speech-detection model for Bravo-1 and Bravo-3. (G) the false positive and negative rates as a function of the probability threshold used in the speech verification classifier. (H) accuracy of the 10-word classifier during baseline and listening blocks across 6 pseudo-blocks. (I) electrode contributions for the 10-word classifier for Bravo-1 and Bravo-3. (J) scatter plot comparing electrode contributions for the 10-word classifier during online evaluations with the listening distractor versus without. (K) FPR of the decoding system when using the full system and a speech-detection model only trained on speech. (L) the number of false positives using the full system and a speech-only speech detection model during long periods of listening and reading.

FIGS. 50A to 50M depict shared and distinct cortical activations for reading, listening, and attempted speech. (A) electrode heat maps for responsiveness in each task measured by non-parametric tests of pre-trial vs post go-cue mean high-gamma amplitude (HGA). (B) electrodes that have task modulation for reading, listening, and attempted speech visualized alongside electrodes that encode movements of the vocal-tract articulators during continuous speech and hand in Bravo-3. (C)-(E) Two-sided Wilcoxon rank-sum test for each electrode are plotted for reading and speech (C) speech and listening (D), and listening and reading (E). (F) example evoked response potentials (ERPs) for an electrode in Bravo-3 that has significant task modulation across attempted speech, reading, and listening. (G) the mean high-gamma amplitude, theta power, and beta power during reading, listening, and attempted speech across electrodes with significant task modulation for listening, reading, and attempted speech. (H) classification accuracy for the speech-verification model. (I) electrode contributions to the full speech-verification model in Bravo-3. (J) a confusion matrix for predictions from the full speech-verification model in Bravo-3. (K)-(M) the same as (H)-(J) for Bravo-1.

FIGS. 51A to 51D illustrate distinct representations for attempted speech, listening, and reading on speech cortex. (A) classification accuracy by anatomical region on the isolated-word set during reading, listening, and attempted speech. (B) for the Bravo-3 temporal-lobe attempted speech and listening classification models, accuracy is shown for evaluating models across tasks. (C) electrode contributions for Bravo-3 temporal-lobe attempted speech and listening classification models. (D) scatterplot of electrode contributions from (C) across temporal-lobe electrodes.

FIGS. 52A to 52E demonstrate the role for shared electrodes in speech-motor planning. (A) example evoked response potentials (ERPs) from two electrodes in Bravo-3 that are task-modulated during reading. (B) average HGA for each reading-responsive electrode from the isolated-word set and false fonts. (C) average HGA for each reading-responsive electrode from the isolated-word set and sentences. (D) electrode contributions for attempted-speech models, trained on different windows of time around the go-cue. (E) average electrode contribution for postcentral, precentral, and temporal-lobe electrodes in each decoding window.

FIGS. 53A to 53D illustrate the effect of speech decoding system ablations on false positive and negative performance. (A) the false positive rate (FPR) of the system with only a speech-detection model trained only on attempted speech, the full model, and the full speech decoding system during baseline, listening, and reading blocks. (B) the true positive rate (TPR) for each of the system ablations in (A) during baseline, listening, and reading blocks. (C) the absolute number of false positives for each of the system ablations during “long” distractor blocks of listening and reading, where there were no speech attempts and only the distractor. (D) the number of time points (at 200 Hz) that had a speech probability greater than the probability threshold of 0.5 for each of the system ablations and “long” distractor blocks.

FIG. 54 depicts speech-verification model performance for different time windows around detected events for models in accordance with embodiments of the invention.

FIGS. 55A to 55C illustrate temporal dynamics of shared activity during attempted speech, listening, and reading. (A) mean evoked response potentials (ERPs) for attempted-speech, listening, and reading. (B) the maximum HGA evoked by attempted speech, reading, and listening for each tri-function electrode. (C) the evoked HGA 500 ms before the onset/go-cue for attempted speech, reading, and listening for each tri-function electrode.

FIG. 56 provides electrodes with comparable HGA for reading words and false fonts localized around the frontal eye fields in accordance with embodiments of the invention.

FIG. 57 provides anatomical characteristics of electrodes important for decoding attempted speech pre go-cue and after the go-cue in accordance with embodiments of the invention.

FIG. 58 illustrates a sample artifact that occurred during three trials of online evaluation with Bravo-1 in accordance with embodiments of the invention.

FIGS. 59A to 59G demonstrate the implementation of a bilingual speech neuroprosthesis in accordance with embodiments of the invention. (A) schematic diagram of the bilingual decoding system. (B) word error rates with the phrase test set, calculated using shuffled neural data, neural decoding from the RNN without language modeling, and the full online system with language modeling. (C) language classification accuracy for chance, neural-only, and online results. (D) the decoding rate compared to the participant's communication speed with his alternative augmentative communication (AAC) strategy. (E) the language-classification accuracy as a function of word position in a phrase. (F) phrase likelihood scores from GPT2 (large language model) for trials where the language is correctly and incorrectly classified. (G) word error rates, as in (B) when the target language is manually set rather than freely decoded.

FIGS. 60A to 60G provide offline characterizations of the bilingual classification algorithms in accordance with embodiments of the invention. (A) 10-fold cross-validation classification accuracy for English words, Spanish words, and across both languages. (B) classification of words in English, Spanish, and across both languages for 48 days without retraining or recalibration of the system. (C) classification performance before (n=5 days) and after (n=5 days) a 30-day break in recording without retraining. (D) electrode contributions for models trained only on English or Spanish words, separated by neural-feature type (HGA and LFS). (E) relationship between 128 HGA (left) and LFS (right) electrode contributions for Spanish and English models. (F) selected portion of the confusion matrix between bilingual words, highlighting confusability. (G) multiple regression models were fit to predict confusability between a pair of words from their acoustic similarity, semantic similarity, and whether the words are in the same language.

FIGS. 61A to 61J demonstrate shared articulatory representations in speech-motor cortex across languages. (A) large stimulus set of unique words and phrases used to cover a larger articulatory space in each language, relative to the vocabulary used for core phrase-decoding (FIG. 59). (B) standard deviation of the average high-gamma amplitude (HGA) from 0 to 2 seconds (relative to the visual go cue) for English and Spanish phrases across each electrode. (C) sample evoked response potentials (ERPs) to English and Spanish phrases for two electrodes noted in (B). (D) relationship between the maximum HGA for each electrode during English and Spanish phrases. (E) relationship between the HGA standard deviation (as in (B)) for English and Spanish phrases for each electrode. (F) relationship of the correlation of ERPs within a language to the correlation of ERPs between languages for each electrode. (G) 10-fold cross-validation (CV) classification accuracy for classifying each phrase as English or Spanish. (H) stimulus set designed to probe a shared articulatory, syllabic representation between languages. (I) sample ERPs from an electrode indicated in (B) for different syllables in the same language and a shared syllable in different languages. (J) 10-fold CV syllable-classification accuracy across training and testing paradigms.

FIGS. 62A to 62F demonstrate rapid transfer learning between languages in accordance with embodiments of the invention. (A) schematic depiction of the paradigm used to evaluate transfer learning between languages. (B) learning curves for fine-tuning and evaluating on a new Spanish vocabulary. (C) learning curves for fine-tuning and evaluating on a new English vocabulary. (D) schematic depiction of the paradigm used to evaluate the effect of acoustic similarity between the train and fine-tune set on transfer learning efficacy. (E) mel-cepstral distortion (MCD, as in FIG. 60G) between each word in the acoustically similar or different training sets with the corresponding word in the fine-tune/test set. (F) learning curves for fine-tuning and evaluating on the “Fine-tune and test set,” defined in (D), with transfer learning from the acoustically similar and different models.

FIG. 63 illustrates the effect of the amount of pretrain data on transfer learning efficacy for models in accordance with embodiments of the invention.

FIG. 64 illustrates the effect of pre-training with silently attempted speech data for models in accordance with embodiments of the invention.

FIG. 65 demonstrates the performance of an attempted speech model using windows of various input lengths in accordance with embodiments of the invention.

FIG. 66 provides a comparison of all online-evaluation sentences and the AAC evaluation subset in accordance with embodiments of the invention.

### DETAILED DESCRIPTION

Methods of assisting individuals with communication are provided. In the disclosed methods, cortical activity from a region of the brain associated with movement, speech production, and/or language perception is recorded while an individual attempts to perform an action (e.g., to say words, express an emotion, perform a movement, etc.). Deep learning computational models are used to detect and decode the attempted action from the recorded brain activity. Decoding of actions from brain activity may be aided by the use of self-supervised machine learning techniques, which discretize each action into one or more action representations that serve as an effective intermediary for decoding neural activity patterns and features into meaningful action outputs. In addition, methods for synthesizing decoded actions into audio and/or visual stimuli are provided, allowing for more naturalistic and expressive communication for individuals who are unable to speak or experience other mobility limitations that inhibit full embodied communication.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### DEFINITIONS

The term “communication” includes word-based communication such as verbal communication including spoken speech, spelling of words, and production of text (e.g., controlling a personal device to generate email or text via attempts to speak) as well as action-based communication such as through attempted non-speech motor movement. Attempted speech may include vocalized speech, which may or may not be intelligible, or non-vocalized speech. Silent-speech attempts are volitional attempts to articulate speech without vocalizing. Attempted non-speech motor movement may include imagined movement without any detectable physical movement. Attempted non-speech motor movements may include, without limitation, imagined head, arm, hand, foot, and leg movements. Attempted non-speech motor movements may be used to indicate the initiation or termination of attempted speech or an attempted action or to control an external device (e.g., for communication with a personal device or software applications or to turn on or off a device). In the disclosed methods, neural activity is recorded during attempted actions whether or not the individual produces any vocal output or detectable motor movement.

The term “communication disorders” is used herein to refer to a group of conditions that affect the ability of a subject to communicate (e.g., using word-based and/or action-based communication). Communication disorders include, without limitation, anarthria, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barré syndrome, Tay-Sachs disease, encephalopathy, central pontine myelinolysis, and other conditions causing dysfunction or paralysis of the muscles of the head, neck, or chest, or arms resulting in anarthria.

The terms “subject”, “individual”, “patient”, and “participant” are used interchangeably herein and refer to a patient having a communication disorder. The patient is preferably human, e.g., a child, an adolescent, an adult, such as a young, middle-aged, or elderly human who may benefit from the systems, devices, and methods disclosed herein for restoring communication. The patient may have been diagnosed as having anarthria or may be paralyzed.

The term “user” as used herein refers to a person that interacts with a device and/system disclosed herein for performing one or more steps of the presently disclosed methods. The user may be the patient receiving treatment. The user may be a health care practitioner, such as the patient's physician.

### Methods

As summarized above, methods of assisting individuals with communication are provided. In the disclosed methods, cortical activity from a region of the brain associated with movement, speech production, and/or language perception is recorded while an individual attempts to perform an action (e.g., to say words, express an emotion, perform a movement, etc.). Deep learning computational models are used to detect and decode the attempted action from the recorded brain activity. In some embodiments, decoding of actions from brain activity is aided by the use of self-supervised machine learning techniques, which discretize each action into one or more action representations that serve as an effective intermediary for decoding neural activity patterns and features into meaningful action outputs. In addition, methods for synthesizing decoded actions into audio and/or visual stimuli are provided, allowing for more naturalistic and expressive communication for individuals who are unable to speak or experience other mobility limitations that inhibit full embodied communication.

### Attempted Actions and Decoded Actions

As described above, embodiments of the methods include recording cortical activity from a region of the brain associated with movement, speech production, and/or language perception while a subject attempts to perform an action. The action attempted by the subject may be an action the subject is unable to perform without aid, or an action the subject has difficulty performing without aid. Attempted actions may include imagined movement of one or more body parts without any detectable physical movement or imagined speech without any discernable noise. Neural activity is recorded during attempted actions whether or not the individual produces any vocal output or detectable motor movement.

In some embodiments, the attempted action includes attempted speech. In these instances, the subject may have difficulty articulating intelligible words. In some embodiments, the subject has a has a low intelligibility for speaking prompted words or sentences (e.g., as measured by speech perception testing (SPT)). For example, the subject may have a speech intelligibility of 87% or less for prompted words, or 78% or less, or 67% or less, or 50% or less, or 10% or less, or 5% or less, or 0%. In some cases, the subject may have a speech intelligibility of 95% or less for prompted sentences, or 89% or less, or 50% or less, or 10% or less, or 5% or less, or 0%. In some embodiments, the subject may have difficulty articulating intelligible words, or may have anarthria, as the result of experiencing a disease or condition. In these instances, the disease or condition may be, but is not limited to, any condition or disease causing dysfunction or paralysis of the muscles of the head, neck, or chest, or arms resulting in anarthria. For example, the disease or condition may be, or may result from, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barré syndrome, Tay-Sachs disease, encephalopathy, central pontine myelinolysis, etc.

In some embodiments, the attempted action includes an attempted movement. In some embodiments, the attempted movement may be an orofacial movement for speech or a non-speech related movement. In these instances, the subject may have difficulty performing the movement. In some embodiments, the subject may have no ability to perform the movement as, e.g., the body part used to perform the movement is missing or is completely paralyzed. In some embodiments, the subject may have difficulty performing the movement, or may be completely unable to perform the movement, as the result of experiencing a disease or condition. In these instances, the disease or condition may be, but is not limited to, any condition or disease reducing the mobility of one or more body parts of the subject. For example, the disease or condition may be, or may result from, strokes, traumatic brain or spinal injuries, amputations, birth defects, cerebral palsy, Friedreich's ataxia, Guillain-Barré syndrome, Lyme disease, spina bifida, arthritis, tendonitis, a tendon or myotendinous tear, a hernia, old age, chronic health problems, etc. In some instances, the subject may experience diplegia, hemiplegia, monoplegia, paraplegia, or quadriplegia.

As described above, the action attempted by the subject may be an action the subject is unable to perform without aid or assistance, or an action the subject has difficulty performing without aid or assistance. In embodiments where the attempted action includes speech, the speech may include a single speech sound (i.e., a single phoneme), a single word, or a single sentence. In other cases, the speech may include multiple speech sounds, words, or sentences. For example, the speech may include a phrase made up of 2 or more words, or 5 or more words, or 10 or more words, or 20 or more, or 50 or more, or 100 or more. The words and/or phrases may be in any language. For example, the words may be in English, Spanish, Mandarin, Dutch, Swahili, Hindi, etc. In some embodiments, spoken phrases or sentences may include words in multiple different languages. In these cases, the words of the phrases/sentences may switch between languages in any manner, e.g., mirroring the nature of a bilingual conversation. In embodiments where the attempted action includes a movement, the attempted action may include a single movement. For example, the attempted action may consist of the abduction, adduction, flexion, extension, and/or circumduction of a body part or a single orofacial movement. In other cases, the attempted action may include multiple movements. For example, the attempted action may include making a facial expression, shooting a basketball, opening a door, walking, hand gestures including multiple finger flexions, a shoulder shrug, a dance move, a hug, etc. In these instances, the attempted action may include 2 or more movements, or 3 or more movements, or 5 or more, or 10 or more, or 15 or more, or 20 or more.

In some embodiments, the attempted action may be selected from a limited group or set of actions. The number of actions included is preferably large enough to create a meaningful variety of actions but small enough to enable satisfactory neural-based classification performance. In embodiments where the attempted action includes speech, the set of actions may be a set of words or phrases. In other words, the attempted speech may include one or more words from a set of words or one or more phrases from a set of phrases. In some instances, the set of words may include 50 words or more, or 100 words or more, or 300 words or more, or 500 words or more, or 1,000 words or more, or 5,000 words or more, or all the words of a specific languages (e.g., English, Spanish, Mandarin, etc.). In some cases, the set of phrases may include 50 phrases or more, or 100 phrases or more, or 300 phrases or more, or 500 phrases or more, or 1,000 phrases or more. In some embodiments, the word or phrase set is adapted or configured for a specific use. For example, the word or phrase set may include words or phrases useful for expressing basic emotions, communicating caregiving needs, discussing a specific interest or hobby (e.g., a sport, a fandom, a videogame, music, etc.), discussing a profession (e.g., accounting, a field of scientific research, law, finance, etc.), or interacting with other individuals in a specific context (e.g., shopping at a store, interacting in a specific videogame or metaverse, etc.). In some cases, the word or phrase set may be adapted or configured for a specific individual based on the linguistic ability of the individual. For example, word or phrase sets including words from multiple different languages may be created for individuals having the ability to communicate in multiple different languages (such as, e.g., a word set including both English and Spanish words for a bilingual speaker of both languages). In some instances, the word or phrase set may be adapted or configured based on the region an individual is from and/or based on the vocabulary of an individual (i.e., the number of words known to the individual).

In some embodiments, the subject is limited to 2 or more word or phrase sets, such as 5 or more, or 10 or more, or 20 or more, or 50 or more, or 100 or more, or 1,000 or more. In some cases, additional word or phrase sets may be created for the subject as needed. In some instances, the subject is able to switch between word or phrase sets as desired. For example, the subject may switch from a word set adapted for communicating caregiving needs to a word set adapted for playing a specific video game when the subject begins playing the video game. In some cases, a specific non-speech related movement may be used to indicate a switch between word or phrase sets, followed by, e.g., the attempted speech of a word associated with a specific word or phrase set to be switched to. In some instances, a switch between word sets may be automatically initiated based on the context of a conversation.

As discussed above, the attempted action may be selected from a limited group or set of actions. In embodiments where the attempted action includes a movement, the set of actions may be a set of movements. In other words, the attempted action may include one or more movements from a set of movements (i.e., each action in the action set may include one or more movements). In some instances, the set of movements may include 3 movements or more, or 6 movements or more, or 9 movements or more, or 20 movements or more, or 50 movements or more, or 100 movements or more, or 1,000 movements or more. In some embodiments, the movement set is adapted or configured for a specific use. For example, the movement set may include movements useful for expressing basic emotions, communicating caregiving needs, performing tasks associated with a specific interest or hobby (e.g., a sport, a videogame, music, etc.), performing tasks associated with a profession (e.g., accounting, lab work, law, finance, etc.), or interacting with other individuals in a specific context (e.g., shopping at a store, interacting in a specific videogame or metaverse, etc.). In some cases, the movement sent may be adapted or configured to communicate a non-verbal language. For example, the movement set may be configured to communicate via American Sign Language (ASL) and, as such, may include movements associated with specific words or phrases in ASL. In these instances, the movement set may be adapted to include specific word or phrase associated movements in a similar manner as the word or phrase sets for attempted speech discussed above (i.e., based on the linguistic abilities of an individual, specific conversational contexts, etc.).

In some embodiments, the subject is limited to 2 or more movement sets, such as 5 or more, or 10 or more, or 20 or more, or 50 or more, or 100 or more, or 1,000 or more. In some cases, additional movement sets may be created for the subject as needed. In some instances, the subject is able to switch between movement sets as desired. For example, the subject may switch from a movement set adapted for communicating caregiving needs to a movement set adapted for playing a specific video game when the subject begins playing the video game. In some cases, a specific movement that is not included in any movement set, or not included in the presently selected movement set, may be used to indicate a switch between movement sets, followed by, e.g., the attempted speech of a word associated with a specific movement set to be switched to. In some instances, a switch between movement sets may be automatically initiated based on the context of a conversation.

In some embodiments, multiple different attempted action sets (i.e., word or phrase sets and/or movement sets) may be combined, e.g., such that one or more body parts of a subject may simultaneously be limited via a plurality of different attempted action sets. In some embodiments, a single part of the body may be limited via multiple different attempted action sets. For example, the hands of an individual may simultaneously be limited to attempting to communicate via ASL and attempting to interact with a specific virtual or real-world environment. In some instances, different parts of the body may be limited via different attempted action sets. For example, the legs of an individual may be limited to attempting to walk and/or run while the facial features of an individual may be limited to attempting to display specific emotions.

In some embodiments, the attempted action is performed in order to synthesize audio, generate text, or control an electronic output device or system to perform one or more actions. By electronic output device or system is meant any device or system capable of being controlled using electrical signals. For example, the device may be a prosthetic limb having an electric motor, a visual display (e.g., a computer monitor, a television, a virtual or augmented reality headset, etc.), a program or software configured to control one or more visual displays, a robot, a speaker, etc. In some embodiments, the electronic output device or system includes a computer system and the attempted action performed by the subject controls a cursor (via, e.g., a virtual mouse), controls a keyboard (e.g., a virtual keyboard), and/or enters computer commands. The output device or system may receive electrical control signals through a variety of means. In some embodiments, the output device or system may receive electrical control signals directly, e.g., through a wire. In other embodiments, the output device or system may receive electrical control signals by converting an electromagnetic or ultrasound wave into an electronic control signal. For example, the output device or system may be controlled using Bluetooth®, Wi-Fi, cell phone towers and/or satellites (e.g., via Global System for Mobile communications (GSM) or Starlink), etc. In some cases, the electrical control signals are digital signals.

In some embodiments, the electronic output device or system is controlled to perform a different action than the action attempted by the subject. For example, the attempted action performed by the subject may be a hand gesture and the action of the electronic output device or system may be to power on or off. In some embodiments, attempted communicatory actions beyond or different from attempted speech (e.g., facial movements, non-facial movements, silence, hand gestures conveying sign language, etc.) are decoded into speech audio or text. In this way, any method of communication (e.g., overt, covert, silent, etc.) capable of being employed by a subject (e.g., a human) is encompassed within the invention. In some instances, attempted actions performed by the subject that are capable of being decoded quickly and/or are easily distinguishable from one another (i.e., via brain electrical signals using, e.g., the methods as described in greater detail below) may be used to control the one or more actions performed by the electronic output device or system. For example, hand movements that are easily distinguishable from attempted speech, and from other hand movements in a movement set (i.e., as described above), may be used to control a mobility scooter or a prosthetic arm. In some embodiments, the electronic output device or system is controlled to perform the same or a similar action as the action attempted by the subject. For example, the attempted action may be waving, and a prosthetic limb may be controlled to wave.

In some embodiments, multiple simultaneous actions may be used to control the electronic output device or system. In these cases, some, all, or none of the simultaneously attempted actions may be the same or similar to the action the electronic output device or system is controlled to perform. For example, the attempted speech of a subject may be used to control the audible words (e.g., sound frequences) produced by a speaker while finger movements may be used to control the volume of the speaker. In some embodiments, the subject may be able to control multiple different electronic output devices or systems simultaneously. For example, the subject may be able to simultaneously generate noise via a speaker by attempting to speak and move a mobility scooter by attempting to point in a direction.

As discussed above, the attempted action may be performed in order to synthesize audio or generate text. In embodiments where the attempted action includes speech, the attempted speech may be performed in order to generate text or synthesize speech audio such as, e.g., electronic speech audio (i.e., speech audio in a computer readable form).

As discussed above, the attempted action may be performed in order to control an electronic output device or system to perform one or more actions. In some embodiments, the electronic output device or system includes an avatar such as, e.g., a humanoid avatar. In some instances, the avatar may be a physical avatar such as, e.g., an animatronic robot. In some embodiments, the avatar is a virtual avatar. In these instances, the output device or system may include, but is not limited to, a visual display configured to present the avatar (and, e.g., actions performed by the avatar or text generated from attempted actions), a loudspeaker configured to produce sounds made by the avatar (such as, e.g., electronic speech audio synthesized using attempted speech by the subject, as described in greater detail below), and/or an avatar-animation system that includes a computer program for designing and/or animating the avatar. In these embodiments, the avatar may be controlled to perform the same action as the action attempted by the subject, or the same action as at least one of the simultaneously attempted actions of the subject.

In some embodiments, the actions performed by the avatar include speech, orofacial movements for speech, and/or non-speech movements (e.g., non-speech communicative gestures). The speech may include any of the words or phrases as discussed above. The orofacial movements may include, but are not limited to, one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction. The non-speech movements may include, but are not limited to, the abduction, adduction, flexion, extension, and/or circumduction of one or more body parts. In some instances, the non-speech movements may include emotional expressions using facial muscles, such as, e.g., happy, sad, and/or surprised expressions. In these instances, the emotional expressions may include different levels of emotion or expression. For example, the emotional expressions may include extremely happy, very happy, and/or somewhat happy expressions.

As discussed above, embodiments of the methods include recording cortical activity from a region of the brain associated with movement, speech production, and/or language perception while a subject attempts to perform an action. In some embodiments, the attempted action includes attempted speech. In these instances, the subject may have difficulty articulating intelligible words. In some embodiments, the attempted action includes an attempted movement. In these instances, the subject may have difficulty performing the movement. In some embodiments, the attempted action may be selected from a limited group or set of actions. In embodiments where the attempted action includes speech, the set of actions may be a set of words or phrases. In some instances, the set of words or phrases may include 1000 words or more. In embodiments where the attempted action includes a movement, the set of actions may be a set of movements. In some instances, the set of movements may include 9 movements or more. In some embodiments, the subject is limited to 2 or more action sets. In some instances, the subject is able to switch between action sets as desired. In embodiments where the attempted action includes speech, the attempted speech may be performed in order to synthesize speech audio such as, e.g., electronic speech audio. In some embodiments, the attempted action may be performed in order to control an electronic output device or system to perform one or more actions. In some embodiments, the electronic output device or system includes an animated humanoid avatar. In some embodiments, the actions performed by the avatar include speech, orofacial movements for speech, and/or non-speech movements. In some instances, actions performed by the avatar are the same actions as the actions attempted by the subject. Brain electrical signal data associated with the attempted movement by the subject may be recorded and used to synthesize speech audio or control the humanoid avatar, as is discussed in greater detail below.

### Recording Brain Electrical Signals

Embodiments of the methods include positioning a neural recording device including an electrode at a location in a region of the brain of the subject to record brain electrical signal data associated with an attempted action by the subject, e.g., as described above. Embodiments of the methods further include positioning an interface in communication with a computing device at a location on the head of the subject, wherein the interface is connected to the neural recording device. Embodiments of the methods further include recording the brain electrical signal data associated with the attempted action by the subject using the neural recording device, wherein the interface receives the brain electrical signal data from the neural recording device and transmits the brain electrical signal data to a processor of the computing device.

As discussed above, embodiments of the methods include positioning a neural recording device including one or more electrodes at a location of the brain of the subject such as, e.g., in a sensorimotor cortex region, to record brain electrical signal data associated with an attempted action by the subject; and positioning an interface in communication with a computing device at a location on the head of the subject. Brain electrical signal data associated with attempted speech and/or an attempted movement by the subject is recorded using the neural recording device, wherein the interface receives the brain electrical signal data from the neural recording device and transmits the brain electrical signal data to a processor programmed to detect an attempted action by the subject and decode the detected action from the recorded brain electrical signal data. In embodiments where the attempted action includes one or more movements, what the movement is (e.g., a finger flexion) and/or specifics of the movement (e.g., the intensity and duration of the movement) may be decoded from the recorded brain electrical signal data. In embodiments where the attempted action includes speech, one or more speech sounds (e.g., words or phrases) may be decoded from the recorded brain electrical signal data.

The recording device may include non-brain penetrating surface electrodes and/or brain-penetrating depth electrodes. In some embodiments, the recording device may include non-brain penetrating surface electrodes. The electrical signals may be recorded using a single electrode, electrode pairs, or an electrode array. In some embodiments, the brain activity is recorded from more than one site. In certain embodiments, brain electrical signal data is recorded from a sensorimotor cortex region of the brain involved in speech processing such as the precentral gyrus, postcentral gyrus, posterior middle frontal gyrus, posterior superior frontal gyrus, or posterior inferior frontal gyrus region, or any combination thereof. In certain embodiments, brain electrical signal data is recorded from a sensorimotor cortex region of the brain involved in movement, such as a region associated with movement of a specific muscle group or area of the body. In some embodiments, the electrode is positioned on the pial surface of the sensorimotor cortex region of the brain.

Positioning an electrode for recording brain activity at specified region(s) of the brain may be carried out using standard surgical procedures for placement of intra-cranial electrodes. As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. In other words, an electrode that is in contact with a region of the brain is physically touching the region of the brain. An electrode in contact with a region of the brain can be used to detect electrical signals corresponding to neural activity associated with attempted speech and/or an attempted movement. Electrodes used in the methods disclosed herein may be monopolar (cathode or anode) or bipolar (e.g., having an anode and a cathode).

In certain embodiments, one or more electrodes are used to record electrical signals for neural activity associated with attempted speech and/or attempted movement in one or more brain regions. An electrode may be placed, for example, in a region of the sensorimotor cortex involved in speech processing such as the superior gyrus, the middle temporal gyrus, the precentral gyrus, and/or the postcentral gyrus regions of the brain. In certain cases, placing the electrode may involve positioning the electrode on the surface of the specified region(s) of the brain. For example, electrodes may be placed on the surface of the brain at the superior gyrus, the middle temporal gyrus, the precentral gyrus, and/or the postcentral gyrus, or any combination thereof. The electrode may contact at least a portion of the surface of the brain at the superior gyrus, the middle temporal gyrus, the precentral gyrus, and/or the postcentral gyrus. In some embodiments, the electrode may contact substantially the entire surface area at the superior gyrus, the middle temporal gyrus, the precentral gyrus, and/or the postcentral gyrus regions of the brain. In some embodiments, the electrode may additionally contact area(s) adjacent to the superior gyrus, the middle temporal gyrus, the precentral gyrus, and/or the postcentral gyrus regions. In embodiments where the neural recording device includes an electrode array, the electrode array may be centered on the central sulcus.

In some embodiments, an electrode array arranged on a planar support substrate may be used for detecting electrical signals for neural activity from one or more of the brain regions specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode or an electroencephalography (EEG) electrode. In some embodiments, the non-brain penetrating electrode may be an ECoG electrode.

In certain cases, placing the electrode at a target area or site (e.g., a neural recording device electrode) may involve positioning a brain penetrating electrode (also referred to as depth electrode) in the specified region(s) of the brain. For example, a depth electrode may be placed in a selected region of the sensorimotor cortex involved in speech processing (e.g., the superior gyrus, the middle temporal gyrus, the precentral gyrus, and/or the postcentral gyrus region) or involved with movement such as, e.g., the movement of a specific muscle group. In some embodiments, the electrode may additionally contact area(s) adjacent to the selected region of the sensorimotor cortex involved in speech processing (e.g., adjacent to the superior gyrus, the middle temporal gyrus, the precentral gyrus, and/or the postcentral gyrus region) or involved with movement such as, e.g., the movement of a specific muscle group. In some embodiments, an electrode array may be used for recording electrical signals at the selected region of the sensorimotor cortex involved in speech processing (e.g., the superior gyrus, the middle temporal gyrus, the precentral gyrus, and/or the postcentral gyrus region) or involved with movement such as, e.g., the movement of a specific muscle group as specified herein.

The depth to which an electrode is inserted into the brain may be determined by the desired level of contact between the electrode array and the brain and the types of neural populations that the electrode would have access to for recording electrical signals. A brain-penetrating electrode array may be obtained from a commercial supplier. A commercially obtained electrode array may be modified to achieve a desired depth of insertion into the brain tissue. In some cases, the electrode may be a non-brain penetrating electrode and the depth to which an electrode is inserted into the brain is negligible and/or is the minimum depth the electrode can be inserted to ensure stable contact with a region of the brain.

The precise number of electrodes contained in an electrode array (e.g., for recording of neural activity associated with an attempted action) may vary. In certain aspects, an electrode array may include 2 or more electrodes, such as 3 or more, 10 or more, 50 or more, 100 or more, 200 or more, 250 or more, 500 or more, including 253 or more, e.g., about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, about 96 to 128 electrodes, about 128 to 196 electrodes, about 196 to 294 electrodes, about 294 to 440 electrodes, or more electrodes. The electrodes may be arranged into a regular repeating pattern (e.g., a grid, such as a grid with about 3 mm center-to-center spacing between electrodes), or no pattern. An electrode that conforms to the target site for optimal recording of electrical signals from neural activity associated with attempted speech and/or an attempted movement by a subject may be used. One such example, is a non-brain penetrating high-density electrode array that consists of 253 disk-shaped electrodes arranged in a lattice formation with 3-mm center-to-center spacing, wherein each electrode has a 1-mm recording-contact diameter and a 2-mm overall diameter. Another example is an electrode with multiple contacts. Yet further, another example of an electrode that can be used in the present methods branched electrode such as a is a 2 or 3 branched electrode to cover the target site.

In some embodiments, a high-density ECoG electrode array is used to record electrical signals from neural activity associated with attempted speech and/or an attempted movement by a subject. For example, a high-density ECoG electrode array may include at least 100 electrodes, at least 128 electrodes, at least 196 electrodes, at least 253 electrodes, at least 294 electrodes, at least 500 electrodes, or at least 1000 electrodes, or more. In some embodiments, the electrode center-to-center spacing in a high-density ECoG electrode array ranges from 250 μm to 4 mm, including any electrode center-to-center spacing within this range such as 250 μm, 300 μm, 350 μm, 400 μm, 500 μm, 550 μm, 600 μm, 650 μm, 700 μm, 800 μm, 900 μm, 1 mm, 1.5 mm, 2 mm, 2.5 mm, 3 mm, 3.5 mm, or 4 mm. In some embodiments, a high-density ECoG micro-electrode array is used. ECoG micro-electrode arrays may include electrodes having a diameter of 250 μm or less, 230 μm or less, or 200 μm or less, including electrodes having a diameter ranging from 150 μm to 250 μm, including any diameter within this range such as 150, 160, 170, 180, 190, 200, 210, 220, 230, 240, or 250 μm. For a description of high-density ECoG electrode arrays and micro-electrode arrays, see, e.g., Muller et al. (2015) Annu Int Conf IEEE Eng Med Biol Soc 2016:1528-1531; Chiang et al (2020) J. Neural Eng. 17:046008; Escabi et al. (2014) J. Neurophysiol. 112(6): 1566-1583; herein incorporated by reference.

The size of each electrode may also vary depending upon such factors as the number of electrodes in the array, the location of the electrodes, the material, the age of the patient, and other factors. In certain aspects, each electrode has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further includes mapping the brain of the subject to optimize positioning of an electrode. In some embodiments, the positioning of an electrode is optimized to detect brain activity features associated with attempted speech and/or an attempted movement by the subject and to achieve optimal decoding of attempted speech or the attempted movement. For example, patterns of electrical signals in specific frequency ranges (e.g., alpha, delta, beta, gamma, and/or high gamma) may be used for detecting attempted speech and/or an attempted movement and decoding speech sounds and movements intended by the subject. Thus, electrodes may be positioned to optimize detection and/or decoding of brain activity in specific frequency ranges to restore communication to a subject who has a communication disorder or mobility to a subject who has a mobility disorder.

In certain aspects, the methods and systems of the present disclosure may include recording brain activity, for example, electrical activity in the ventral sensorimotor cortex, where patterns of gamma-frequency neural activity associated with words, phrases, and sentences of attempted speech, orofacial movements associated with attempted speech, or movements of a specific muscle or muscle group may be detected. In certain cases, electrical activity from a plurality of locations in the sensorimotor cortex may be measured. In some embodiments, electrical activity in the high gamma frequency range (such as 70 Hz to 150 Hz) and/or the low frequency range (such as 0.3 Hz to 100 Hz) may be measured. In some embodiments, electrical activity in the high gamma frequency range (such as 70 Hz to 150 Hz) and the low frequency range (such as 0.3 Hz to 100 Hz) may be measured the superior gyrus, the middle temporal gyrus, the precentral gyrus, and/or the postcentral gyrus region, or any combination thereof.

Detection of brain activity may be performed by any method known in the art. For example, functional brain imaging of neural activity may be carried out by electrical methods such as electrocorticography (ECoG), electroencephalography (EEG), stereoelectroencephalography (sEEG), magnetoencephalography (MEG), single photon emission computed tomography (SPECT), as well as metabolic and blood flow studies such as functional magnetic resonance imaging (fMRI), positron emission tomography (PET), functional near-infrared spectroscopy (fNIRS), and time-domain functional near-infrared spectroscopy. In some embodiments, the sensorimotor cortex (such as, e.g., a specific region of the sensorimotor cortex including one or more of the superior gyrus, the middle temporal gyrus, the precentral gyrus, and/or the postcentral gyrus region) is mapped to determine optimal positioning for electrodes to detect neural activity associated with attempted speech and/or an attempted movement by the subject. One or more regions may be implanted with a neural recording device including electrodes to measure electrical signals from neural activity associated with attempted speech and/or an attempted movement.

In some cases, electrical activity in one or more locations in the brain may be measured not only during attempted speech or an attempted movement but also during a period extending from just prior to attempted speech or attempted movement (i.e., period of preparation for speech or movement) to a period just after attempted speech or movement (i.e., rest period after attempted speech or movement). Assessment of the accuracy of the decoding of speech or movement from neural activity at a particular site may be determined by comparing decoded speech sounds to the intended speech sounds of the subject or comparing decoded movements to the intended movements of the subject. For example, the patient may communicate the correct intended sounds or movements using an assistive typing device. Both detection of the onset and offset of speech or movement events and speech sound and movement classification accuracy from decoding neural activity may be evaluated. False positives include detected speech or movement events that are not associated with a true speech sound or movement production attempt and false negatives include speech sound or movement production attempts that are not associated with a detected speech or movement event. Lower error rates in detection of speech or movement events and decoding of speech sounds or movement from neural activity indicate better performance. In certain cases, the placement of electrodes or the number of electrodes may be altered to improve detection of electrical signals and decoding of attempted speech and/or movement by the subject.

Application of the methods may include a prior step of selecting a patient for implantation with a neural recording device based on need as determined by clinical assessment of the severity of the communication or mobility disorder and the desire for assistance with communication or mobility, and may also include cognitive assessment, anatomical assessment, behavioral assessment and/or neurophysiological assessment. Patients who have difficulty with communication may be implanted with a neural recording device to assist communication, as described herein.

Embodiments of the methods may include implanting an interface capable of communication with a computing device in the head of the subject or placing the interface on the head of the subject to provide an externally accessible platform through which brain electrical signals can be acquired from the neural recording device and transmitted to a data processor for decoding. In some embodiments, the interface includes a percutaneous pedestal connector anchored in the cranium of the subject. The interface can be connected, for example, to a computing device such as a computer or a handheld computing device (e.g., cell phone or tablet) with a detachable digital connector and cable. Alternatively, the interface may be connected to a computing device wirelessly. In some embodiments, the interface includes a first wireless communication unit in communication with a computing device including a second wireless communication unit. In some embodiments, the first wireless communication unit utilizes a wireless communication protocol using an electromagnetic carrier wave (e.g., a radio wave, microwave, or an infrared carrier wave) or ultrasound to transfer data from the interface to the computing device including the second wireless communication unit. Brain-computer interfaces are commercially available, including the Neuroport™ system from Blackrock Microsystems (Salt Lake City, Utah), See also, e.g., Weiss et al. (2019) Brain-Computer Interfaces 6:106-117; herein incorporated by reference.

The processor may be provided by a computer or a handheld computing device (e.g., cell phone or tablet) programmed to decode the attempted speech and/or attempted movement from the recorded brain electrical signal data.

As discussed above, a neural recording device may be used to record brain electrical signal data associated with an attempted action (e.g., attempted speech or an attempted movement) by the subject. In certain embodiments, a series of go cues is provided to the subject indicating when the subject should initiate attempted an attempted action. In some embodiments, the series of go cues are provided visually on a display. Each go cue may be preceded by a countdown to the presentation of the go cue, wherein the countdown for the attempted action is provided visually on the display and automatically started after each go cue. In some embodiments, the series of go cues are provided with a set interval of time between each go cue, which may be adjustable by the user. In certain embodiments, the processor is programmed to use the recorded brain electrical signal data within a time window following a go cue. In some embodiments, the processor is programmed to use the recorded brain electrical signal data within a time window before a go cue and following a go cue.

As discussed above, embodiments of the methods include positioning a neural recording device including an electrode at a location in a region of the brain of the subject to record brain electrical signal data associated with an attempted action by the subject. Embodiments of the methods further include positioning an interface in communication with a computing device at a location on the head of the subject, wherein the interface is connected to the neural recording device, and recording the brain electrical signal data associated with the attempted action by the subject using the neural recording device, wherein the interface receives the brain electrical signal data from the neural recording device and transmits the brain electrical signal data to a processor of the computing device. In certain embodiments, brain electrical signal data is recorded from a sensorimotor cortex region of the brain involved in movement, such as a region associated with movement of a specific muscle group or area of the body. In certain embodiments, brain electrical signal data is recorded from a sensorimotor cortex region of the brain involved in speech processing such as the precentral gyrus, postcentral gyrus, posterior middle frontal gyrus, posterior superior frontal gyrus, or posterior inferior frontal gyrus region, or any combination thereof. In some embodiments, the recording device may include a non-brain penetrating surface array of electrodes. The number of electrodes contained in the electrode array may be 250 or more electrodes. In some cases, the non-brain penetrating electrode array may be an electrocorticography (ECoG) electrode array. In some embodiments, electrical activity in the high gamma frequency range (such as 70 Hz to 150 Hz) and/or the low frequency range (such as 0.3 Hz to 100 Hz) may be measured. In some cases, electrical activity in one or more locations in the brain may be measured during a period extending from just prior to attempted speech or movement to a period just after attempted speech or movement. Embodiments of the methods further include implanting an interface capable of communication with a computing device in the head of the subject or placing the interface on the head of the subject to provide an externally accessible platform through which brain electrical signals can be acquired from the neural recording device and transmitted to a data processor for decoding. The processor may be provided by a computer or a handheld computing device (e.g., cell phone or tablet) programmed to decode the attempted speech and/or attempted movement from the recorded brain electrical signal data. The processor is programmed to use a machine learning model to decode one or more speech sounds or one or more electronic output device or system actions (e.g., one or more actions of an animated humanoid avatar) from the recorded brain electrical signal data as is discussed in greater detail below.

Decoding Actions from Brain Electrical Signals

Embodiments of the methods include decoding one or more speech sounds, text (i.e., one or more words or sentences), or one or more electronic output device or system actions (e.g., one or more actions of an animated humanoid avatar or prosthetic arm) from brain electrical signal data recorded, e.g., as described above using a machine learning system. The decoding machine learning system, in accordance with embodiments of the methods, may vary and may include, but is not limited to, any of the models discussed below. Decoding of speech sounds, text, and/or actions from brain activity may be aided by the use of self-supervised machine learning techniques, which discretize each speech sound or action into one or more discrete or continuous speech units or action representations that serve as an effective intermediary for decoding neural activity patterns and features into meaningful action outputs. In some embodiments, the methods further include training the decoding machine learning system to perform one or more tasks facilitating the decoding of one or more speech sounds or one or more electronic output device or system actions from the recorded brain electrical signal data. In some embodiments, the training may further include validating and testing.

The term “decoding machine learning system” or “decoding system” as used herein refers to the machine learning model, or group of connected or interconnected models, used to generate one or more speech sounds, text, or one or more electronic output device or system actions from brain electrical signal data recorded, e.g., as described above (i.e., the model(s) used for decoding brain electrical signal data). The decoding machine learning system or decoding system may include other machine learning architectures (e.g., encoders) and may perform multiple different techniques (e.g., encoding) during the process of generating speech sounds/text/output actions from brain electrical signal data. Further, models not explicitly referred to as the “decoding machine learning system” or “decoding system” may be a component of the decoding machine learning system or decoding system based on context. For example, a model referred to as a “neural encoder” is a component of the decoding system if it actively takes part in the aforementioned decoding of speech/text/actions from brain electrical signal data (i.e., is part of the process of decoding after the model(s) of the decoding system have completed training). Further, the decoding machine learning system or decoding system may not be the only machine learning model(s) that performs the technique of decoding. As such, the terms “decoding machine learning system” or “decoding system” are used for the sake of clarity and are not meant to inherently be limiting beyond the definition supplied above.

The term “encoding machine learning system” or “encoding system” as used herein refers to the machine learning model, or group of connected or interconnected models, that, e.g., discretizes reference speech sounds or reference electronic output device or system actions (e.g., reference avatar animations), or creates embedding spaces using said reference sounds or actions, in order to generate intermediate representations for decoding neural activity patterns and features into meaningful action outputs. In some embodiments, the encoding machine learning system or encoding system may include other machine learning architectures (e.g., decoders) and may perform multiple different techniques (e.g., decoding) during the process of generating effective intermediate representations useful for decoding recorded neural activity. In some cases, the decoding machine learning system may include one or more components of the encoding machine learning system. For example, an encoding system may include both an encoder and a decoder and the decoding system may include the encoder and/or the decoder of the encoding system. Further, the encoding machine learning system or encoding system may not be the only machine learning model(s) that performs the technique of encoding. As such, the terms “encoding machine learning system” or “encoding system” are used for the sake of clarity and are not meant to inherently be limiting beyond the definition supplied above.

The term “synthesizing machine learning system” or “synthesizing system” as used herein refers to the machine learning model, or group of connected or interconnected models, that synthesizes one or more speech sounds or speech audio from intermediary representations generated using the decoding system. In this way, the synthesizing system may be a component of the decoding system (i.e., the decoding system may include the synthesizing system) in embodiments wherein the decoding system is used to generate speech sounds from brain electrical signal data. The synthesizing machine learning system or synthesizing system may not be the only machine learning model(s) that performs the technique of synthesizing. As such, the terms “synthesizing machine learning system” or “synthesizing system” are used for the sake of clarity and are not meant to inherently be limiting beyond the definition supplied above.

The decoding machine learning system, in accordance with embodiments of the methods, may vary and may include, but is not limited to, any of the models discussed below or any standard machine learning model known in the art, as well as combinations thereof, capable of performing the decoding tasks described below.

In some embodiments, the decoding machine learning system may include a Logistic Regression, K-Nearest Neighbors, Decision Tree, Random Forest and/or XGBoost model. In some embodiments, the decoding machine learning system may include an artificial neural network (NN) (e.g., a convolutional NN (CNN)). In some embodiments, the machine learning system includes a deep learning model. In these cases, the model may be three or more layers deep, such as five or more layers deep, or ten or more, or twelve or more. In some embodiments, the decoding machine learning system is configured to process sequential input data. In these instances, the decoding machine learning model may include, or be based on, a recurrent neural network (RNN) model or a transformer model (e.g., encoders, decoders and/or attention). In embodiments where the decoding system includes an RNN, the RNN may include, e.g., long short-term memory (LSTM) architecture, gated recurrent units (GRUs), and/or an attention mechanism. In some embodiments, the decoding machine learning system may include an RNN with one or more layers of GRUs, such as 2 layers or more of layers of GRUs, or 3 or more of layers of GRUs, or 5 or more of layers of GRUs.

The decoding machine learning system may learn from the contextual information of the brain electrical signal data and, e.g., may learn from the past to present context of the data and/or the present to past context of the data. In some embodiments, the decoding system may learn from both the past to present context and the present to past context of the brain electrical signal data (i.e., the decoding machine learning model may be bidirectional). For example, the decoding system may include, or be based on, e.g., a bidirectional LSTM model, an RNN model with an attention, a convolutional recurrent neural network model with an attention (CRNN-A), a transformer model, a bidirectional RNN model, or a Transducer (e.g., an RNN Transducer) model. In some embodiments, the bidirectional decoding system includes, or is based on, an RNN model, and the bidirectional RNN model may include one or more layers of GRUs as described above. In some embodiments, the decoding machine learning system such as, e.g., a bidirectional decoding system including a bidirectional RNN model having one or more layers of GRUs, may include one or more convolutional layers. In some cases, the decoding machine learning system includes a linear readout layer. In some cases, a layer of the decoding system may comprise a plurality of hidden units such as, e.g., 10 or more units, or 50 or more units, or 100 or more, or 500 or more.

In certain embodiments, brain electrical signal data is decoded into one or more speech sounds or electronic output device or system actions (e.g., one or more actions of an animated humanoid avatar) using intermediate representations. In some cases, multiple intermediate representations may be combined to generate or decode a single speech sound or electronic output device or system action. In other instances, a single intermediate representations may be used to generate or decode a single speech sound or electronic output device or system action. In some embodiments, the intermediate representations are a set of discrete action representations. In other words, the decoding system may decode one or discrete action representations from the brain electrical signal data, and the one or discrete action representations may then be used to decode the one or more speech sounds or electronic output device or system actions. The set of discrete action representations may be obtained using self-supervised, supervised, or unsupervised machine learning techniques. In some cases, the set of discrete action representations may be obtained using self-supervised machine learning techniques. In some embodiments, the discrete action representations are generated during self-supervised training of an encoding machine learning system (i.e., one or more machine learning models) using reference speech sounds (e.g., the speech sounds of the word or phrase sets as discussed above) and/or speech sounds from a large database or reference electronic output device or system actions (e.g., reference avatar animations). In some instances, the encoding machine learning system may include an artificial neural network (ANN). In some embodiments, the encoding system includes a convolutional element such as, e.g., one or more convolutional layers.

In embodiments where the attempted action is attempted speech, the intermediate representations may be discrete speech units. In these instances, the discrete speech units may be generated by applying a pretrained (i.e., using self-supervised machine learning techniques) encoding machine learning system to reference speech sounds (i.e., the speech sounds of the word or phrase sets as discussed above). In these cases, the encoding system may include a transformer encoder. For example, the encoding machine learning system may include a Hidden-Unit Bidirectional Encoder Representations from Transformers (HuBERT) model. In some embodiments, the HuBERT model generates 50 or more different discrete speech units, such as 80 or more different discrete speech units, or 100 or more different discrete speech units.

In embodiments where the attempted action is performed in order to control the actions or animations of a computer-generated humanoid avatar, the intermediate representations may be discrete action representations. In these instances, the discrete action representations may be generated during self-supervised training of an encoding machine learning system using reference avatar animations. In these instances, the encoding machine learning system may include a neural network. In some embodiments, the neural network includes one or more components of a variational autoencoder and/or may include one or more rectified linear unit (ReLU) activations. In some embodiments, the encoding machine learning system may include an encoder and a decoder, e.g., for encoding each of the reference avatar animations into one or more discrete action representations (encoder) and for decoding one or more discrete action representations into an avatar animation (decoder). For example, the encoding machine learning system may include a vector-quantized variational autoencoder (VQ-VAE) model.

As described above, brain electrical signal data may be decoded into one or more speech sounds or electronic output device or system actions (e.g., one or more actions of an animated humanoid avatar) using intermediate representations. In some embodiments, the intermediate representations are a set of continuous action representations. In other words, the decoding system may decode one or continuous action representations from the brain electrical signal data, and the one or continuous action representations may then be used to decode the one or more speech sounds or electronic output device or system actions. In some embodiments, the continuous action representations are generated using embedding. In these embodiments, the embeddings/embedding space may be generated using any context. For example, the embedding space may be generated using semantic context (i.e., wherein words of similar meanings tend to be near each other in the embedding space), movement context (i.e., wherein similar types of movements, or movements using similar muscles, tend to be near each other in the embedding space), sound context (i.e., wherein similar types of sounds tend to be near each other in the embedding space), and/or any other context. The embedding space for continuous action representations may be obtained using self-supervised, supervised, or unsupervised machine learning techniques. In some cases, the embedding space for continuous action representations may be obtained using supervised machine learning techniques. In some embodiments, the embedding space for continuous action representations is generated during supervised training of an encoding machine learning system using reference speech sounds (e.g., the speech sounds of the word or phrase sets as discussed above) and/or speech sounds from a large database or reference electronic output device or system actions (e.g., reference avatar animations). In some instances, the encoding machine learning system may include an artificial neural network (ANN). In some embodiments, the encoding system includes a convolutional element such as, e.g., one or more convolutional layers.

In some embodiments, the decoding machine learning system may include a predictor model configured to predict a future intermediate representation or decoding system output (e.g., speech sound, text, and/or action) based, e.g., on previous representations/outputs output by the decoding system. In some cases, the predictor model may predict the most likely next intermediate representation or produce a probability distribution (i.e., discrete or continuous) over a number or range of possible future intermediate representations using, e.g., previously output representations. For example, in embodiments of the decoding system configured to decode speech sounds or text via discrete representations, the predictor model may include a language model (e.g., BERT or ChatGTP) configured to predict the next word, or intermediate representation thereof, the subject will attempt to speak based, e.g., on what words would form a coherent sentence. In some embodiments, the decoding machine learning system includes a neural encoder (e.g., including one or more components of an encoding system as described above) configured to output one or more intermediate representations such as, e.g., a probability distribution of the most likely intermediate representations, based on recorded brain electrical signal data. In some embodiments, the decoding machine learning system includes a predictor model, a neural encoder, and a joiner module configured to combine the outputs of the predictor model and the neural encoder. In this way, the decoder system may be able to generate intermediate representations based both on contextual information (e.g., semantic context) and brain electrical signals. In some instances, the decoder system may include an RNN Transducer.

In some embodiments, the decoding machine learning system may be configured to decode speech sounds and/or text from multiple different languages simultaneously. In other words, the user does not need to pre-specify which language intend to communicate in, and the system instead infers the desired language from their neural activity. In some embodiments, the decoding system may decode words from a multilingual vocabulary set as the user attempts to speak the words. In some embodiments, the decoding system may perform multilingual decoding of electrical brain signal data using multilingual linguistic representations that are shared across languages. These multilingual linguistic representations may include phonemes, syllables, word pieces, or other linguistic units. The decoded representations of linguistic units can vary across a variety of forms, including, but not limited to, a time series of predicted linguistic-unit probabilities. In some embodiments, one or more language models (e.g., natural language models) are used to score decoded candidate linguistic speech units, words, or sentences (e.g., using linguistic context specific to each language) in order to facilitate selection and output of the most likely decoded speech unit, word, or sentence.

In some embodiments, the decoding system may include multiple different machine learning models each configured to generate context or information via electrical brain signals associated with a different type or category of attempted action. These different types or categories of attempted actions may include, e.g., the attempted actions of different attempted action sets as described above. In some cases, each of two or more models of the decoding system are configured to generate information from the attempted action of a different part or region of the body. For example, the decoding system may include, e.g., a neural encoder trained to produce representations from neural activity features associated with attempted foot movements and a neural encoder trained to produce representations from neural activity features associated with attempted arm movements. In some embodiments, each of two or more models of the decoding system are configured to generate information from different categories of attempted action performed by the same body part. For example, the decoding system may include, e.g., a neural encoder trained to produce representations from neural activity features associated with attempted ASL hand movements and a neural encoder trained to produce representations from neural activity features associated with attempted cursor control movements. In some embodiments, the decoding system may include a joiner module configured to combine (and, e.g., weight) the outputs of the different machine learning models in order to decode speech sounds, text, and/or electronic output device actions from recorded brain electrical signal data.

In some embodiments, the decoding system may be configured decode multiple outputs simultaneously, e.g., from context or information generated by one or multiple machine learning models (e.g., one or multiple neural encoders). For example, a user may attempt to speak while imagining moving their hand to simultaneously facilitate text decoding (e.g., entering a text message into a computer application) and cursor control (e.g., moving a computer cursor around on a screen), wherein the decoding system includes a first neural encoder for the attempted speech and a second neural encoder for the imagined hand movements. In another embodiment, a user may attempt to speak in order to simultaneously control an avatar and a keyboard, e.g., via a single neural encoder. In this way, the decoding system may be used to control or generate any number of different outputs or effectors using any number of different inputs or user intents (e.g., attempted action sets). In some cases, the decoding system may generate/control 1 output based on 2 or more inputs, or 2 or more outputs based on 1 input, or 3 or more outputs based on 1 input, or 1 output based on 3 or more inputs, or 5 or more outputs based on 1 input, or 1 output based on 5 or more inputs, or 2 or more outputs based on 2 or more inputs, or 3 or more outputs based on 3 or more inputs, or 5 or more outputs based on 2 or more inputs, or 2 or more outputs based on 5 or more inputs, etc.

In some embodiments, the decoding system may include an intent detection system comprising one or more machine learning models trained to differentiate neural activity (e.g., brain electrical signal data) associated with volitional attempts or intents of a specific activity (e.g., the attempted movements of a specific movement set or attempted speech of a word or phrase set, as described above) from neural activity related to irrelevant activities. In some embodiments, the intent detection system may be configured to increase the accuracy of the decoding system (e.g., in real-world environments where environmental noise or other irrelevant activities or distractors may be present) and/or volitionally engage and/or disengage the decoding system via the neural activity (e.g., brain electrical signal data) of a user. For example, the decoding system may be inactive until it is activated by the intent detection system when neural features (e.g., HGA and/or LFS) associated with attempted speech are detected. In some embodiments, the intent detection system may be trained via neural activity collected as a user engages in activities unrelated to intended actions. For example, an intent detection system used for detecting neural activity associated with attempted speech may be trained using data collected as a user reads text on a screen or passively listens to speech played via loudspeaker, e.g., in order to better differentiate attempted speech from other language associated tasks. In some cases, a model of the intent detection system may be trained to distinguish time windows of neural activity associated with a specific activity from time windows of neural activity associated with a different activity.

As discussed above, in some embodiments an intent detection system may be used to volitionally engage and disengage the decoding system via brain electrical signal data. In this way, the decoding system may be utilized by a subject as it is needed simply by attempting to speak (e.g., without any setup steps), allowing for the subject to more easily initiate spoken conversation or respond in real time to spontaneous interactions. In some embodiments, the decoding system may decode brain electrical signal data into one or more speech sounds or electronic output device or system actions in real-time, with low latency. In some embodiments, low latency is achieved by continuously streaming small increments of data through a transducer model, as described above. In these cases, the increments may be 500 ms or less, or 100 ms or less, or 80 ms or less, or 50 ms or less, or 10 ms or less.

As discussed above, the methods further include training the decoding machine learning system to perform one or more tasks facilitating the decoding of one or more speech sounds or one or more electronic output device or system actions from the recorded brain electrical signal data. In certain embodiments, the training includes: obtaining reference output device or system actions for a plurality of actions; encoding each reference action into a temporal sequence of discrete action representations using the encoding machine learning system (or, e.g., generating embeddings using the encoding machine learning system if continuous action representations are utilized); recording the brain electrical signal data associated with attempted action by the subject for each one of the plurality of actions; training the decoding machine learning system to perform one or more tasks facilitating the decoding of one or more electronic output device or system actions from the recorded brain electrical signal data using the action representations derived from the reference output device or system actions and the brain electrical signal data associated with the attempted action for each one of the plurality of actions. In some embodiments, the plurality of actions consist of the actions of an action set as described above.

In some embodiments, the decoding machine learning system is trained to predict the most likely discrete action representation associated with a segment of electrical signal data using the action representations derived from the reference output device or system actions and the brain electrical signal data associated with the attempted action for each one of the plurality of actions. In some embodiments, the decoding machine learning system is trained to predict the probability of each discrete action representation for a segment of electrical signal data. In some instances, the decoding machine learning system is trained to learn mappings between neural activity patterns of electrical signals in the brain electrical signal data and the discrete action representations. In some embodiments, device or system actions include avatar animations. In some embodiments, the avatar animations are of the avatar performing the same action as the action attempted by the subject. In certain embodiments, the reference avatar animations are obtained from an avatar-animation system.

In some embodiments, the decoding machine learning system is trained to predict the most likely continuous action representation associated with a segment of electrical signal data using the action representations (e.g., embeddings) derived from the reference output device or system actions and the brain electrical signal data associated with the attempted action for each one of the plurality of actions. In some embodiments, the decoding machine learning system is trained to predict the probability of different regions of an embedding space for a segment of electrical signal data. In some instances, the decoding machine learning system is trained to learn mappings between neural activity patterns of electrical signals in the brain electrical signal data and the continuous action representations (e.g., embeddings).

In embodiments where the attempted action is attempted speech, the training may include: obtaining reference electronic speech waveforms for a plurality of phrases; encoding each reference speech waveform into a temporal sequence of discrete speech units (or, e.g., generating embeddings using the encoding machine learning system if continuous action representations are utilized) using the encoding system; recording the brain electrical signal data associated with attempted speech by the subject for each one of the plurality of phrases; training the decoding machine learning system to perform one or more tasks facilitating the decoding of one or more speech sounds (or, e.g., text) from the recorded brain electrical signal data using the discrete speech units (or, e.g., embeddings) derived from the reference speech waveforms and the brain electrical signal data associated with the attempted speech for each one of the plurality of phrases. In some embodiments, the plurality of phrases consist of the words or phrases of a word or phrase set as described above. In certain embodiments, the reference electronic speech waveforms are obtained from a recruited speaker. In some embodiments, the reference electronic speech waveforms are obtained using a text-to-speech algorithm.

In some embodiments, the decoding machine learning system is trained to predict the most likely discrete speech unit associated with a segment of electrical signal data using the discrete speech units derived from the reference speech waveforms and the brain electrical signal data associated with the attempted speech for each one of the plurality of phrases. In some embodiments, the decoding machine learning system is trained to predict the probability of each discrete speech unit for a segment of electrical signal data. In some instances, the decoding machine learning system is trained to learn mappings between neural activity patterns of electrical signals in the brain electrical signal data and the discrete speech units.

In some embodiments, the decoding machine learning system is trained to predict the most likely continuous speech representation associated with a segment of electrical signal data using the speech representations (e.g., embeddings) derived from the reference speech waveforms and the brain electrical signal data associated with the attempted speech for each one of the plurality of phrases. In some embodiments, the decoding machine learning system is trained to predict the probability of different regions of a word embedding space for a segment of electrical signal data. In some instances, the decoding machine learning system is trained to learn mappings between neural activity patterns of electrical signals in the brain electrical signal data and the continuous speech representations (e.g., word embeddings).

In some embodiments, the training algorithms and hyperparameters used to control the training of the decoding system may depend on, e.g., the nature or architecture of the decoding machine learning system model(s), the tasks the machine learning model(s) is trained to perform, the desired accuracy or efficiency of the decoding machine learning system, and/or the nature or size of the training data set (e.g., the reference speech waveforms or reference avatar animations). In some embodiments, algorithms or techniques are used (e.g., during training) to account for the differences in timing between reference speech waveforms or reference electronic output device or system actions (e.g., reference avatar animations) and the brain electrical signal data associated with the attempted action (i.e., attempted speech or an attempted movement). In some embodiments, a CTC loss function is used during training. In some cases, algorithms or techniques are used during training that correct for overfitting issues such as, e.g., dropout layers.

In embodiments wherein a decoding system is trained to decode speech sounds or text from electrical brain signal data, transfer learning may be used to train the decoding system for a language using a different decoding system trained using a different language. For example, a neural-decoding model may first be trained using brain electrical signal data and associated metadata from one language before the weights resulting from the aforementioned training are used to initialize the weights of a subsequent model trained using brain electrical signal data and activity from a second language. In some cases, transfer learning may be used to train a decoding system for a second language after the decoding system has first been trained using a first language. In some cases, multilingual transfer learning, as described above, may be used to reduce the amount of training data required to train a decoding system.

As discussed above, algorithms or techniques may be used to account for the differences in timing between reference speech waveforms or reference electronic output device or system actions (e.g., reference avatar animations) and the brain electrical signal data associated with the attempted action (i.e., attempted speech or an attempted movement). In some cases, a silence/pause or blank token is used in order to account for the differences in timing between reference speech waveforms or reference electronic output device or system actions. For example, the intermediate representations (e.g., discrete speech units, continuous action representations, etc.) may include a representation of the lack of speech or the lack of an action (e.g., representing periods of time where the subject is not attempting speech or an action) that can be referred to as a blank token. In embodiments wherein the intermediate representations are continuous, the blank token may be zero or may be the origin in the coordinate plane of the embedding space. In some cases, connectionist temporal classification (CTC) decoding is used with the blank token. In some cases, an RNN-transducer is used with the blank token. In some embodiments, training methods that do not rely on alignment are utilized (e.g., supervised machine learning techniques). In embodiments wherein supervised machine learning techniques are utilized, the labels may be generated in any fashion, e.g., from the task, using forced alignment, or using unsupervised learning. In some cases, breaks between actions (e.g., words or sentences for attempted speech) may be decoded using a blank token. In these embodiments, the decoding system may be continuously pinged or polled in order to determine when the blank or silence token is being decoded from the brain electrical data by the decoding system. In some cases, the decoding system may be polled every 500 ms or less, such as every 100 ms or less, or every 10 ms or less, or every 5 ms or less, or every 1 ms or less. In these cases, if the blank or silence token is determined to have been decoded by the decoding system for a certain number of polls in a row, the end of an action, or group of actions, may be determined (e.g., the end of a word, sentence, or attempted speech). In some cases, a certain number of blank or silence tokens may initiate the collapse of a CTC search beam. In some cases, a certain number of blank or silence tokens may initiate the next frame of an RNN-transducer search function.

In some embodiments, the training may further include testing the trained decoding machine learning system or decoding machine learning systems. By testing in this context is meant evaluating the trained decoding machine learning system using, e.g., reference data (e.g., reference speech waveforms or reference avatar animations) different from the reference data used for training after the machine learning system has finished training. The testing may use one or more metrics to evaluate the performance of the trained decoding machine learning system. In some embodiments, the metric may be used to determine if the trained decoding machine learning system performs sufficiently using, e.g., a predetermined threshold (i.e., requirement). In these instances, if the trained decoding machine learning system does not meet the predetermined threshold, the model may be discarded and/or another model may be trained. In embodiments where another decoding machine learning system is trained, one or more of the model architecture, training and/or the training data set may be modified prior to training. In some instances, decoding machine learning models are trained until a trained machine learning models meets the predetermined threshold. The division between the training set of reference data and the testing set of reference data may vary. In some cases, roughly 80% of the reference data may be used for training and roughly 20% for testing. In some instances, roughly 70% of the reference data may be used for training and roughly 30% for testing.

In some embodiments, the training may further include validating the decoding machine learning model or decoding machine learning models. By validating in this context is meant evaluating the decoding machine learning system during training using reference data different from the reference data used for training and testing. The validating may use one or more metrics to evaluate the performance of the decoding machine learning model or decoding machine learning models. In some embodiments, the validating may be used to, e.g., select model parameters (e.g., select one or more machine learning algorithms to continue training), optimize or tune hyperparameters (e.g., model hyperparameters or algorithm hyperparameters), etc. The division between the training reference data, validating reference data, and testing reference data may vary. In some cases, roughly 80% of the reference data may be used for training, roughly 10% for testing, and roughly 10% for validating.

As discussed above, embodiments of the methods include decoding one or more speech sounds or one or more electronic output device or system actions (e.g., one or more actions of a computer-generated humanoid avatar) from recorded brain electrical signal data using a machine learning model. In some embodiments, the decoding machine learning model may be bidirectional and may include an RNN with one or more layers of GRUs. In certain embodiments, brain electrical signal data is decoded into one or more speech sounds or electronic output device or system actions using a set of discrete action representations (e.g., discrete speech units). The set of discrete action representations may be obtained using self-supervised machine learning techniques. In some embodiments, the discrete action representations are generated during self-supervised training of an encoding machine learning model. In embodiments where the attempted action is attempted speech, the encoding machine learning model may be a Hidden-Unit Bidirectional Encoder Representations from Transformers (HuBERT) model. In embodiments where the attempted action is performed in order to control the actions or animations of a computer-generated humanoid avatar, the encoding machine learning model may be a vector-quantized variational autoencoder (VQ-VAE) model. The methods may further include training the decoding machine learning model to perform one or more tasks facilitating the decoding of one or more speech sounds or one or more electronic output device or system actions from the recorded brain electrical signal data. In some embodiments, the decoding machine learning model is trained to predict the most likely discrete action representation associated with a segment of electrical signal data using the action representations derived from reference data (e.g., reference waveforms or reference output device or system actions) and the brain electrical signal data associated with the attempted action for each one of a plurality of actions. In some embodiments, a CTC loss function is used during training. In some cases, the training may further include validating and testing. An encoding machine learning model or a synthesizing machine learning model may be used to synthesize speech audio (e.g., one or more speech sounds or an electronic speech waveform) or generate electrical control signals for an electronic output device or system from the discrete action representations decoded from the recorded the brain electrical signal data as is discussed in greater detail below.

### Synthesizing Audio/Visual Stimuli and Controlling Electronic Output Devices

Embodiments of the methods may include decoding one or more speech sounds or one or more electronic output device or system actions (e.g., one or more actions of a computer-generated humanoid avatar) from discrete action representations (e.g., discrete speech units) decoded from the recorded the brain electrical signal data using the decoding machine learning model as discussed above. In some embodiments, one or more speech sounds are decoded from discrete speech units using a speech synthesizer. In some embodiments, one or more avatar animations are decoded from discrete action representations using an encoding machine learning model. Embodiments of the methods may further include synthesizing an electronic speech waveform from the decoded speech sounds and/or generating an electrical control signal from the decoded electronic output device or system actions. In some embodiments, the electronic speech waveform may be transformed into the subject's own voice. In some embodiments, electrical control signals may be generated from multiple machine learning models that cause a computer-generated avatar to speak, perform orofacial movements for speech, and perform non-speech associated actions.

As discussed above, one or discrete action representations may be decoded from the brain electrical signal data using the decoding model, and the one or more discrete action representations may then be used to decode the one or more speech sounds or electronic output device or system actions. In embodiments where discrete speech units are decoded from brain electrical signal data associated with attempted speech, the discrete speech units may be decoded into one or more speech sounds and synthesized into an electronic speech waveform using a speech synthesizer. In some embodiments, the speech synthesizer includes a machine learning model. In some embodiments, the synthesizing machine learning model is configured to generate a spectrogram from one or more discrete speech units. In other words, the synthesizing machine learning model may be trained to generate a spectrogram from one or more discrete speech units. In some instances, the spectrogram is a mel spectrogram. In some cases, the synthesizing machine learning model may include a neural network. In these instances, the synthesizing machine learning model may be an RNN such as, e.g., a bidirectional RNN. In embodiments where the synthesizing machine learning model includes a bidirectional RNN, the bidirectional RNN may include GRUs or LSTM. In some cases, the bidirectional RNN includes one or more layers of GRUs. In some embodiments, the synthesizing machine learning model may include one or more convolutional layers and/or may include an attention mechanism. In some cases, the synthesizing machine learning model may include the Tacotron2 model.

In some embodiments, the speech synthesizer further includes a vocoder configured to synthesize an electronic speech waveform from the spectrogram generated using the synthesizing machine learning model. In some instances, the vocoder may include a machine learning model. In some cases, the vocoder machine learning model may include a neural network. In these instances, the neural network may include one or more convolutional layers. In some cases, the vocoder machine learning model may include the WaveGlow model.

In certain embodiments, the electronic speech waveform decoded from recorded brain electrical signal data and synthesized using the speech synthesizer is transformed or converted into a personalized electronic speech waveform. For example, the electronic speech waveform may be transformed such that it resembles the subject's own voice. In some embodiments, the electronic speech waveform is transformed using a machine learning model, such as a machine learning model including a neural network. In some cases, the conversion machine learning model may include an encoder and/or may be a zero-shot learning model. In these instances, the conversion machine learning model may include the YourTTS model.

In some embodiments, the electronic speech waveform decoded from recorded brain electrical signal data and synthesized using the speech synthesizer is converted into an audible speech waveform using a loudspeaker. In some embodiments, an electronic output device or system is controlled using the electronic speech waveform. For example, an animated avatar (such as the humanoid avatar described herein) may be controlled to perform speech associated orofacial movements based on the electronic speech waveform. In some embodiments, the electronic speech waveform is played in sync with a humanoid avatar performing orifical facial movements decoded using a different decoding model than the decoding model used to decode the one or more speech sounds used to synthesize the electronic speech waveform.

In embodiments where discrete action representations are decoded from brain electrical signal data associated with an attempted action (e.g., an attempted movement), the discrete action representations may be decoded into one or more electronic output device or system actions (e.g., one or more actions of a computer-generated humanoid avatar) using a decoder. In some cases, the decoder may be the decoder of the encoding machine learning model as discussed above. For example, a VQ-VAE may be used to discretize or encode each reference electronic output device or system action into one or more discrete action representations and the VQ-VAE's decoder may be used to decode electronic output device or system actions from discrete action representations decoded from recorded brain electrical signal data. The decoded electronic output device or system actions may then be used to control the electronic output device or system (i.e., to perform the decoded action). For example, the decoded electronic output device or system actions may be used to generate electrical control signals that are transmitted to the electronic output device or system.

In some embodiments, the attempted actions performed by the subject and the actions of the device or system include both speech associated actions and non-speech communicative gestures. In some embodiments, the decoding machine learning model is trained to discriminate between actions performed by different regions of the body and/or between speech associated actions and non-speech communicative gestures.

In certain embodiments, the electronic output device or system is controlled using actions decoded from multiple different decoding machine learning models. In some embodiments, the actions decoded from the different decoding machine learning models occur concurrently (i.e., the attempted actions associated with the decoded actions occur concurrently) and the electronic output device or system is controlled to perform the actions simultaneously. For example, multimodal speech decoding may be used to display animations of a humanoid avatar performing orofacial speech movements using a first decoding machine learning model (e.g., as described above), audibly play a speech waveform synthesized using a second decoding machine learning model (e.g., as described above), and display text of the speech using a third decoding machine learning model (see, e.g., the Examples section below) simultaneously as a subject attempts to speak.

In some embodiments, the multiple decoding machine learning models each are trained using a different action set as described above and, e.g., are adapted for a specific region of the body (e.g., the subject's body and the avatar's body) or for a specific purpose. In some cases, the subject may be able to switch between action sets associated with specific regions of the body and with communication in a specific context as desired. For example, the subject may switch to a word or phrase set adapted for playing basketball for generating audible speech and orofacial movements, and to an action set adapted for playing basketball for lower limb movements when playing a basketball video game by controlling the humanoid avatar.

In some embodiments, one decoding machine learning model may affect the manner in which the avatar performs an action decoded from a different machine learning model. In some embodiments, separate decoding machine learning models are trained for speech and orofacial movements for speech. In these embodiments, a decoded non-speech communicative gesture affects how the avatar performs a decoded speech associated action. For example, a decoded emotional expression may affect the inflection of decoded speech performed by an avatar.

As discussed above, embodiments of the methods may include decoding one or more speech sounds or one or more electronic output device or system actions from discrete action representations decoded from the recorded the brain electrical signal data using the decoding machine learning model. In embodiments where discrete speech units are decoded from brain electrical signal data associated with attempted speech, the discrete speech units may be decoded into one or more speech sounds and synthesized into an electronic speech waveform using a speech synthesizer. In some embodiments, the speech synthesizer includes a machine learning model trained to generate a mel spectrogram from one or more discrete speech units. In some cases, the synthesizing machine learning model may include the Tacotron2 model. In some embodiments, the speech synthesizer further includes a vocoder configured to synthesize an electronic speech waveform from the spectrogram generated using the synthesizing machine learning model. The electronic speech waveform decoded from recorded brain electrical signal data and synthesized using the speech synthesizer may be transformed or converted into a personalized electronic speech waveform using a zero-shot learning model such as, e.g., the YourTTS model. In embodiments where discrete action representations are decoded from brain electrical signal data associated with an attempted action (e.g., an attempted movement), the discrete action representations may be decoded into one or more actions of a computer-generated humanoid avatar using the decoder of the encoding machine learning model discussed above. The decoded avatar actions may then be used to control the avatar by generating electrical control signals that are transmitted to the device or system generating the avatar. In certain embodiments, the avatar is simultaneously controlled using actions decoded from multiple different decoding machine learning models. In some embodiments, one decoding machine learning model may affect the manner in which the avatar performs an action decoded from a different machine learning model. The speech sounds generated from recorded brain electrical signal data and the avatar controlled using recorded brain electrical signal data have a variety of applications, as discussed in greater detail below.

### Avatar Applications and Environments

Embodiments of the methods may include methods of applying the speech sounds generated from recorded brain electrical signal data and the avatar controlled using recorded brain electrical signal data. In some embodiments, the speech sounds generated from recorded brain electrical signal data are used as the voice of the avatar controlled using recorded brain electrical signal data. In some embodiments, a virtual environment is created for the avatar, or the avatar is configured to interact in a virtual environment. In some cases, the avatar may be used for communication, entertainment, work, or therapy. The avatar may be highly personalized according to the needs and/or desires of the subject.

In some embodiments, the computer-generated avatar is used by the subject to communicate with one or more individuals in person (e.g., using a TV screen, projector, computer monitor, or augmented reality goggles, glasses, or contacts). In some cases, the avatar is used by the subject to communicate with one or more individuals in an interactive virtual environment (e.g., using one or more TV screens, projectors, computer monitors, or virtual reality goggles, glasses, or contacts). For example, the avatar may be used by the subject to communicate with other individuals in a metaverse, or in an online video game. In these instances, the other individuals may play the video game or interact in the metaverse using a personal computer or a gaming console (e.g., using an Xbox or PlayStation® controller).

In some embodiments, the actions performed by the avatar are determined using recorded brain electrical signal data (e.g., as described above) and the virtual environment the avatar is rendered or generated in. For example, an attempted hand turn by the subject may result in the avatar opening a door when the avatar is within a certain proximity to the door and may result in the avatar waving when the avatar is not adjacent to a door. In some embodiments, the actions performed by the avatar are determined using recorded brain electrical signal data (e.g., as described above) and the previous actions performed by the avatar. For example, multiple consecutive attempted finger snaps may result in the avatar performing a dance.

In some embodiments, the computer-generated avatar is used by the subject for therapy such as, e.g., physical therapy. In these instances, the physical therapy involves regaining mobility of a body part after an injury. For example, by watching the avatar perform leg movements when the subject attempts leg movements the subject may take advantage of neuroplasticity in order to regain leg mobility.

In some embodiments, the computer-generated avatar is used by the subject to control one or more electronic devices in the subject's environment such as, e.g., one or more electronic devices in the same room as the subject. For example, the subject may turn on a light by controlling the avatar to turn on the light in augmented reality. The electronic devices controlled by the avatar may include, but are not limited to, one or smart home appliances, or one or more electronic motors configured to move objects in the subject's environment.

In some embodiments, the computer-generated avatar is personalized. In these instances, one or more physical characteristics of the avatar's body (e.g., facial feature shapes, height, weight, skin color, hair color, eye color, etc.) and/or the avatar's outfits (e.g., shirts, hats, pants, skirts, shoes, socks, necklaces, earrings, etc.) may be customizable by or for the subject.

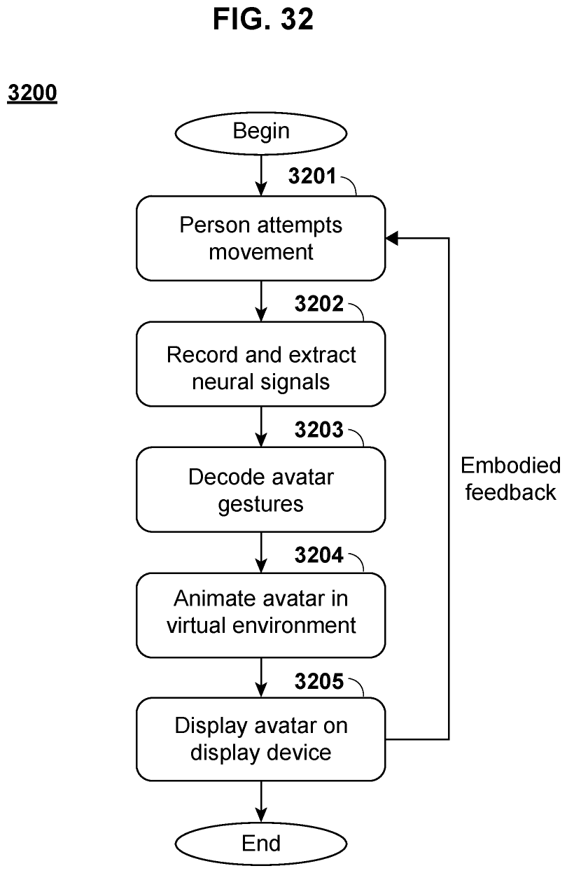

FIG. 32 provides a flow diagram depicting a method of controlling a fully embodied virtual avatar using recorded brain electrical signal data in accordance with an embodiment of the invention. At step 3201, the subject attempts a movement. The movement may be a hand or arm movement such as a wave performed in order to acknowledge another individual in the same physical or virtual environment as the subject. At step 3202, the neural activity occurring as a result of the subject attempting the movement is recorded, e.g., using a non-penetrating high-density ECoG electrode array positioned on the sensorimotor cortex of the subject's brain, and high-gamma activity and low-frequency signals are extracted to produce brain electrical signal data associated with the attempted action. At step 3203, avatar gestures are decoded from the brain electrical signal data using the decoding machine learning model and the decoder of the encoding machine learning model as described above. At step 3204, the decoded avatar gestures are used to animate an avatar in a virtual environment and at step 3205 the animated avatar and, e.g., the virtual environment are displayed on a visual display device. The subject may then attempt another movement using feedback obtained by watching the displayed avatar animation.

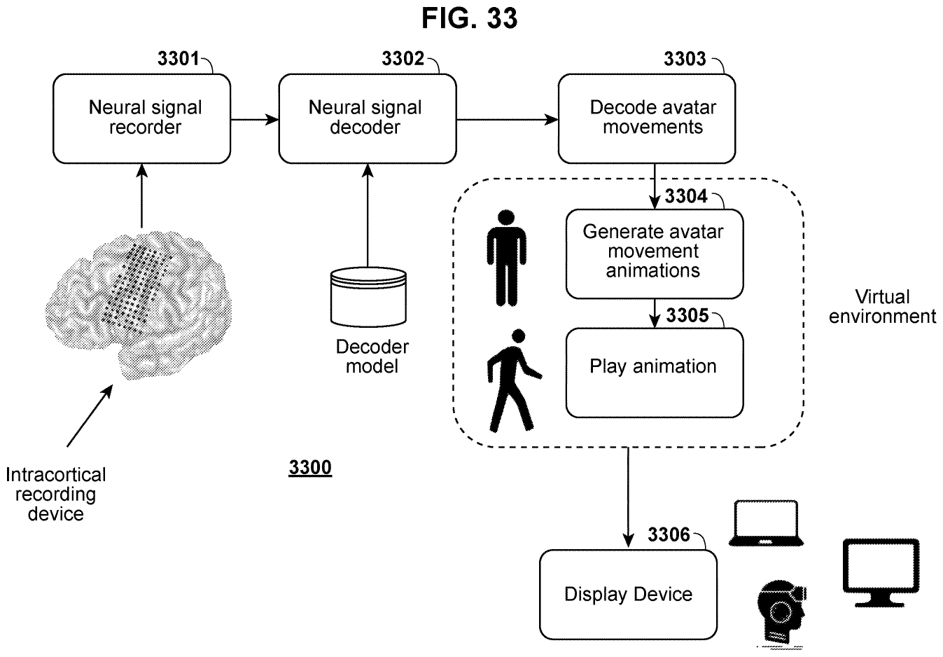

FIG. 33 provides a flow diagram depicting a method of controlling and displaying a fully embodied virtual avatar using recorded brain electrical signal data in accordance with an embodiment of the invention. At step 3301, the neural activity occurring as a result of the subject attempting a movement is recorded, e.g., using an intracortical non-penetrating high-density ECoG electrode, and high-gamma activity and low-frequency signals are extracted to produce brain electrical signal data associated with the attempted action. At step 3302, the decoding machine learning model decodes one or more discrete action representations from the brain electrical signal data. At step 3303, a decoder such as, e.g., the decoder of the autoencoder used to generate the discrete action representations from reference avatar animations is used to decode the one or more discrete action representations (i.e., the representations decoded from the brain electrical signal data in step 3302) into one or more avatar animations. At step 3304, the decoded avatar movements are used to generate an avatar movement animation and at step 3205 the is played. At step 3304, the virtual environment, including the avatar animation, is reproduced on a display device.

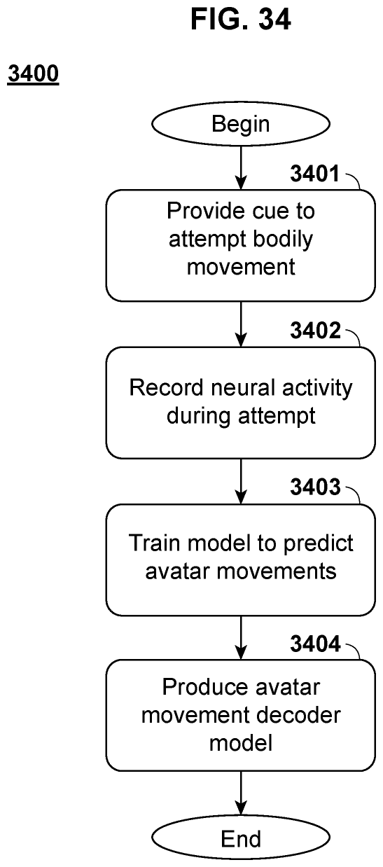

FIG. 34 provides a flow diagram depicting a method for training a machine learning model to predict avatar movements using recorded neural activity in accordance with an embodiment of the invention. At step 3401, a go cue is provided to the subject indicating when the subject should initiate an attempted action. The go cue may be provided visually on a display and may be preceded by a countdown to the presentation of the go cue. At step 3402, the neural activity occurring as a result of the subject attempting a movement is recorded, e.g., using an intracortical non-penetrating high-density ECoG electrode, and high-gamma activity and low-frequency signals are extracted to produce brain electrical signal data associated with the attempted action. In some embodiments, the processor is programmed to use the recorded brain electrical signal data within a time window before the go cue and following the go cue. At step 3403, a decoding machine learning model and, e.g., an autoencoder are trained to predict avatar movements from the recorded brain electrical signal data (e.g., within a time window before the go cue and following the go cue). Training for the decoding model may include: obtaining reference avatar animations for a plurality of movements; encoding each reference animation into a temporal sequence of discrete action representations using the autoencoder; training the decoding machine learning model to predict the most likely discrete action representation associated with a segment of electrical signal data using the action representations derived from the reference avatar animations and the brain electrical signal data recorded at step 3402. At step 3404, an avatar movement decoder model is produced that can decode avatar movements from brain electrical signal data.

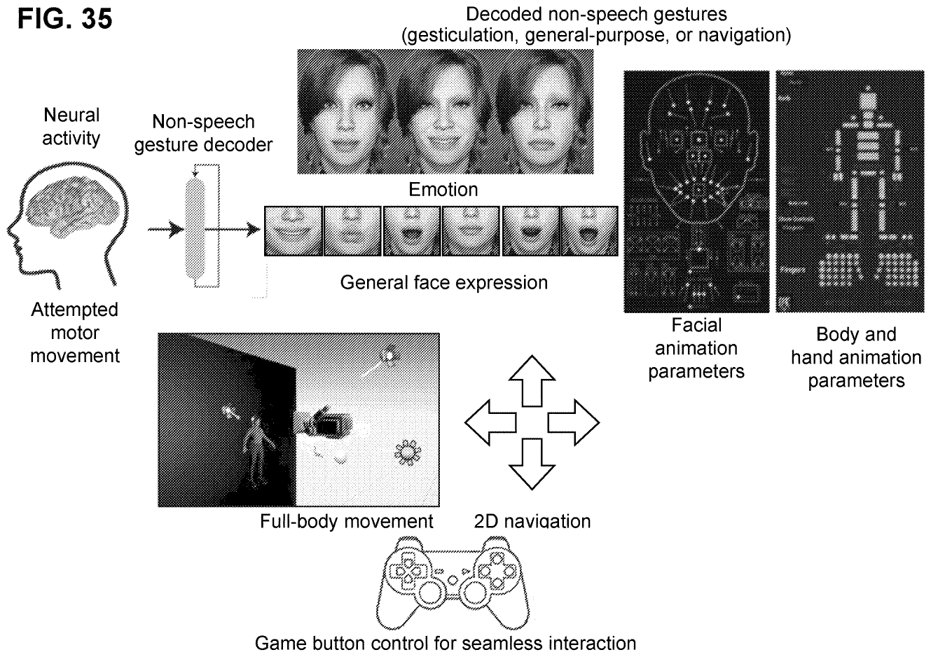

FIG. 35 illustrates an overview of a non-speech communicative gesture neural-decoding pipeline in accordance with embodiments of the invention.

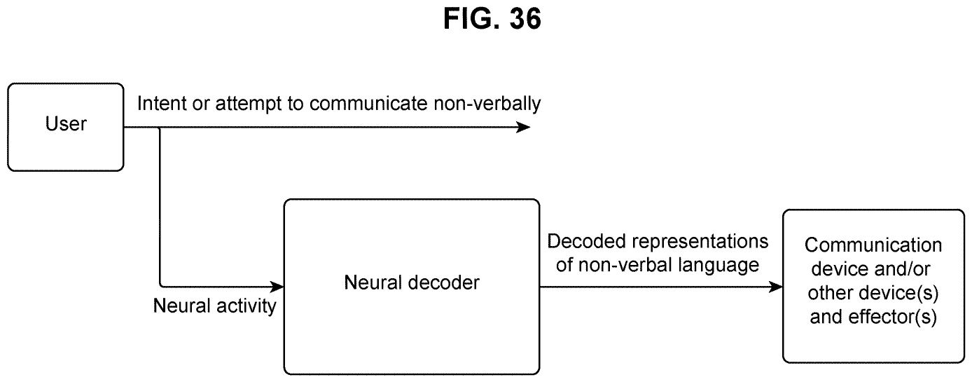

FIG. 36 provides a block diagram of a pipeline for concurrent multi-effector neural-decoding in accordance with embodiments of the invention.

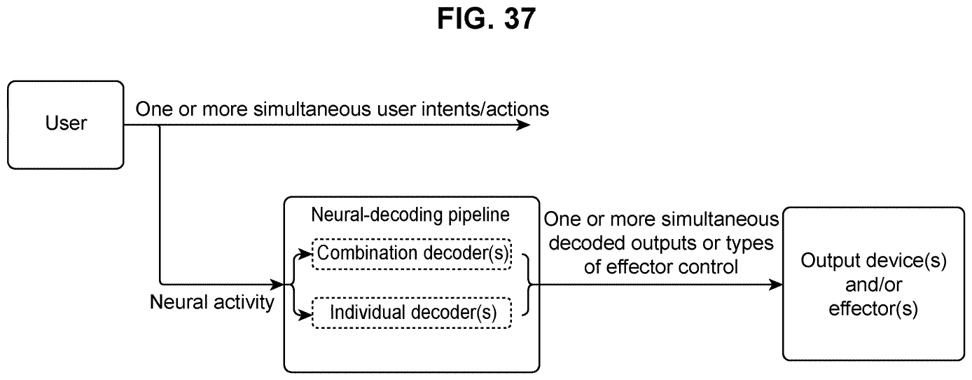

FIG. 37 provides a block diagram of a pipeline for non-verbal linguistic neural-decoding in accordance with embodiments of the invention.

### Systems and Computer Implemented Methods

Aspects of the present disclosure further include systems, such as computer-controlled systems, for practicing embodiments of the above methods. Aspects of the systems may include: a neural recording device including an electrode adapted for positioning at a location in a sensorimotor cortex region of the brain of the subject to record brain electrical signal data associated with attempted speech by the subject; a processor programmed to use a machine learning model to decode one or more speech sounds from the recorded brain electrical signal data; an interface in communication with a computing device, said interface adapted for positioning at a location on the head of the subject, wherein the interface receives the brain electrical signal data from the neural recording device and transmits the brain electrical signal data to the processor; and an audio speaker for playing the one or more speech sounds from the recorded brain electrical signal data.

Aspects of the systems may also include: a neural recording device including an electrode adapted for positioning at a location in a sensorimotor cortex region of the brain of the subject to record brain electrical signal data associated with an attempted action by the subject; a processor programmed to use a machine learning model to decode an avatar animation from the recorded brain electrical signal data; an interface in communication with a computing device, said interface adapted for positioning at a location on the head of the subject, wherein the interface receives the brain electrical signal data from the neural recording device and transmits the brain electrical signal data to the processor; and a display component for displaying the avatar action from the recorded brain electrical signal data.

For example, electrical activity in the high gamma frequency range (such as 70 Hz to 150 Hz) and/or low frequency range (e.g., 0.3 Hz to 100 Hz) from the precentral gyrus, postcentral gyrus, superior temporal gyrus, middle temporal gyrus, or any combination thereof may be recorded with the neural recording device using this system, wherein the interface receives the brain electrical signal data from the neural recording device and transmits the brain electrical signal data to a processor. The processor may run programming for speech sounds or avatar actions from the recorded brain electrical signal data using one or more machine learning models, as described herein.

In some embodiments, a computer implemented method is used for decoding speech sounds from recorded brain electrical signal data associated with attempted speech by a subject. The processor may be programmed to perform steps of the computer implemented method including: receiving the recorded brain electrical signal data associated with the attempted speech by the subject; and decoding one or more speech sounds from the recorded brain electrical signal data using a machine learning model.

In some embodiments, a computer implemented method is used for decoding avatar actions from recorded brain electrical signal data associated with an attempted action by a subject. The processor may be programmed to perform steps of the computer implemented method including: receiving the recorded brain electrical signal data associated with the attempted action by the subject; and decoding one or more electronic output device or system actions from the recorded brain electrical signal data using a machine learning model.

In certain embodiments, the computer implemented method further includes storing a user profile for the subject including information regarding the patterns of electrical signals in the recorded brain electrical signal data associated with attempted speech or an attempted action by the subject.

The recorded brain electrical signal data may be processed in various ways before decoding. For example, data processing may include, without limitation, real-time sample-by-sample processing of neural feature streams, the use of common-average referencing across individual electrode channels, the use of finite impulse response (FIR) filters to perform digital signal filtering, a running sliding-window normalization procedure, e.g., using Welford's method, automatic artifact rejection, and parallelization and linear pipelining to improve computational efficiency. Processing of neural features may be performed in real-time to extract one or more feature streams for use during speech/action decoding. For a description of data processing methods, see, e.g., Moses et al. (2018) J. Neural. Eng. 15(3):036005, Moses et al. (2019) Nat. Commun. 2019 10(1):3096, Moses et al. (2021) N. Engl. J. Med. 385(3):217-227, Sun et al. (2020) J. Neural. Eng. 17(6), and Makin et al. (2020) Nature Neuroscience 23:575-582; herein incorporated by reference in their entireties.

In some instances the systems further include one or more computers for complete automation or partial automation of the methods described herein. In some embodiments, systems include a computer having a computer readable storage medium with a computer program stored thereon.

The methods described herein can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component includes instructions for decoding a speech sound or action from recorded brain electrical signal data associated with attempted speech and/or attempted action by a subject. The computer processor is coupled to the storage component and configured to execute the instructions stored in the storage component in order to receive brain electrical signal data associated with attempted speech by the subject and analyze the data according to one or more algorithms, as described herein. The display component displays the sentence decoded from the recorded brain electrical signal data.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC or an FPGA.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may include any information sufficient to identify the relevant information, such as numbers, descriptive text, proprietary codes, pointers, references to data stored in other memories (including other network locations) or information which is used by a function to calculate the relevant data.

In certain embodiments, the processor and storage component may include multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may include a collection of processors which may or may not operate in parallel.

The system also includes an interface capable of communication with a computing device. The interface may be implanted in the cranium or placed on the head of the subject to provide an externally accessible platform through which brain electrical signals can be acquired from the neural recording device and transmitted to a computing device for decoding. In some embodiments, the interface includes a percutaneous pedestal connector anchored in the cranium of the subject. The interface can be connected, for example, to a computing device such as a computer or a handheld computing device (e.g., cell phone or tablet) with a detachable digital connector and cable. Alternatively, the interface may be connected to a computing device wirelessly. In some embodiments, the interface includes a first wireless communication unit in communication with a computing device including a second wireless communication unit. In some embodiments, the first wireless communication unit utilizes a wireless communication protocol using an electromagnetic carrier wave (e.g., a radio wave, microwave, or an infrared carrier wave) or ultrasound to transfer data from the interface to the computing device including the second wireless communication unit. Brain-computer interfaces are commercially available, including the Neuroport™ system from Blackrock Microsystems (Salt Lake City, Utah), See also, e.g., Weiss et al. (2019) Brain-Computer Interfaces 6:106-117; herein incorporated by reference.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

Components of systems for carrying out the presently disclosed methods are further described in the examples below.

### Kits

Kits are also provided for carrying out the methods described herein. In some embodiments, the kit includes software for carrying out the computer implemented methods for decoding a speech sound or avatar action from recorded brain electrical signal data associated with attempted speech and/or attempted action by a subject, as described herein. In some embodiments, the kit includes a system for assisting a subject with communication as described herein. Such a system may include: a neural recording device including an electrode adapted for positioning at a location in a sensorimotor cortex region of the subject to record brain electrical signal data associated with attempted speech and/or attempted action by the subject; a processor programmed to decode a speech sound or avatar action from the recorded brain electrical signal data according to a computer implemented method described herein; an interface capable of communication with a computing device, said interface adapted for positioning at a location on the head of the subject, wherein the interface receives the brain electrical signal data from the neural recording device and transmits the brain electrical signal data to the processor; and a display component for displaying the speech sound or avatar action decoded from the recorded brain electrical signal data.

In addition, the kits may further include (in certain embodiments) instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The methods, devices, and systems of the invention find use in assisting individuals with communication. In particular, methods, devices, and systems are provided that facilitate full, embodied communication to people living with severe paralysis by restoring the ability to produce speech sounds and facial movements related to speaking, as well as by restoring the ability to perform non-speech communicative gestures. In some embodiments, the methods, devices, and systems of the present disclosure find use in restoring aspects of an individual's personhood and identity by providing them the ability to communicate with naturalistic speed and expressivity, and by providing them with highly personalizable audio-visual representations. Embodiments of the present disclosure enable an individual with a communication or mobility disorder to interface with evolving technology to communicate with family and friends, facilitate community involvement and occupational participation, and engage in virtual, internet-based social contexts (such as social media, videogames, and metaverses). In some embodiments, the subject methods, devices, and systems find use in applications where it is desirable to increase the independence of an individual with a communication or mobility disorder.

The methods, devices, and systems disclosed herein may be used to assist individuals who have difficulty with communication (e.g., using word-based and/or action-based communication) caused by conditions and diseases including, without limitation, anarthria, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barré syndrome, Tay-Sachs disease, encephalopathy, central pontine myelinolysis, and other conditions causing dysfunction or paralysis of the muscles of the head, neck, arms, or chest resulting in anarthria. The methods disclosed herein may be used to restore communication to such individuals and improve autonomy and quality of life.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-406 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of assisting a subject with communication, the method comprising:
   positioning a neural recording device comprising an electrode at a location in a sensorimotor cortex region of the brain of the subject to record brain electrical signal data associated with attempted speech by the subject;
   positioning an interface in communication with a computing device at a location on the head of the subject, wherein the interface is connected to the neural recording device;
   recording the brain electrical signal data associated with attempted speech by the subject using the neural recording device, wherein the interface receives the brain electrical signal data from the neural recording device and transmits the brain electrical signal data to a processor of the computing device; and
   decoding one or more speech sounds from the recorded brain electrical signal data using the processor, wherein the processor is programmed to use a machine learning model for the decoding.
2. The method of aspect 1, wherein the subject has difficulty with said communication because of anarthria, a stroke, a traumatic brain injury, a brain tumor, or amyotrophic lateral sclerosis.
3. The method of aspect 1 or 2, wherein the subject is paralyzed.
4. The method of any of aspects 1-3, wherein the subject has a speech intelligibility of 10% or less for prompted words.
5. The method of any of aspects 1-4, wherein the location of the neural recording device is on a surface of the sensorimotor cortex region or within the sensorimotor cortex region.
6. The method of any of aspects 1-5, wherein the electrode of the neural recording device is positioned on the pial surface of the sensorimotor cortex.
7. The method of any of aspects 1-6, wherein the neural recording device is positioned such that the recording device covers regions associated with speech production and language perception.
8. The method of aspect 7, wherein the covered regions include a middle portion of the superior and/or middle temporal gyrus, the precentral gyrus, and/or the postcentral gyrus.
9. The method of any of aspects 1-8, wherein the neural recording device is centered on the central sulcus.
10. The method of any of aspects 1-9, wherein the neural recording device comprises an electrocorticography (ECoG) electrode array.
11. The method of aspect 10, wherein the ECoG electrode array is a high-density array.
12. The method of aspect 11, wherein the high-density array comprises 200 electrodes or more.
13. The method of any of aspects 10-12, wherein the electrode array comprises non-penetrating surface electrodes.
14. The method of any of aspects 1-13, wherein the interface comprises a percutaneous pedestal connector attached to the subject's cranium.
15. The method of any of aspects 1-14, wherein the interface further comprises a headstage connected to the percutaneous pedestal connector.
16. The method of aspect 15, wherein the headstage processes and digitizes the brain electrical signal data before transmitting the data to the processor.
17. The method of any of aspects 1-16, wherein the electrical signal data comprises high-gamma frequency content features.
18. The method of any of aspects 1-17, wherein the electrical signal data comprises neural oscillations in a range from 70 Hz to 150 Hz.
19. The method of any of aspects 1-18, wherein the electrical signal data comprises low-frequency signals.
20. The method of aspect 19, wherein the electrical signal data comprises neural oscillations in a range from 0.3 Hz to 17 Hz.
21. The method of any of aspects 1-20, wherein the brain electrical signal data is recorded from a sensorimotor cortex region selected from the precentral gyrus, postcentral gyrus, superior temporal gyrus, middle temporal gyrus, or any combination thereof.
22. The method of any of aspects 1-21, wherein the method further comprises mapping the brain of the subject to identify an optimal location for positioning the electrode for recording the brain electrical signals associated with the attempted speech by the subject.
23. The method of any of aspects 1-22, wherein the one or more speech sounds form a word.
24 The method of aspect 23, wherein the one or more speech sounds form a sentence.
25. The method of aspects 23 or 24, wherein the subject is limited to a specified word set for the attempted speech.
26. The method of aspect 25, wherein the word set comprises words for expressing basic concepts and/or caregiving needs.
27. The method of aspects 25 or 26, wherein the word set comprises 100 words or more.
28. The method of aspect 27, wherein the word set comprises 350 words or more.
29. The method of aspect 28, wherein the word set comprises 1000 words or more.
30. The method of any of aspects 25-29, wherein the subject is limited to two or more word sets for the attempted speech.
31. The method of aspect 30, wherein the subject may switch between word sets.
32. The method of any of aspects 1-31, wherein the decoding machine learning model comprises a neural network.
33. The method of aspect 32, wherein the neural network comprises one or more convolutional layers.
34. The method of aspects 32 or 33, wherein the neural network is bidirectional.
35. The method of aspect 34, wherein the neural network is a recurrent neural network (RNN).
36. The method of aspect 35, wherein the RNN comprises gated recurrent units (GRUs) or long-short term memory (LSTM).
37. The method of aspect 36, wherein the RNN comprises GRUs.
38. The method of any of aspects 1-37, wherein one or more discrete speech units of a set of discrete speech units are decoded from the recorded brain electrical signal data.
39. The method of aspect 38, wherein each speech sound is decoded from one or more discrete speech units.
40. The method of aspects 38 or 39, wherein the set of discrete speech units comprises 50 or more discrete speech units.
41. The method of aspect 40, wherein the set of discrete speech units comprises 100 or more discrete speech units.
42. The method of any of aspects 36-39, wherein discrete speech units are continuously decoded at a uniform frequency.
43. The method of aspect 42, wherein the frequency is 50 Hz or more.
44. The method of aspect 43, wherein the frequency is 200 Hz or more.
45. The method of any of aspects 38-44, wherein the set of discrete speech units are generated using an encoding machine learning model.
46. The method of aspect 45, wherein the encoding machine learning model comprises a neural network.
47. The method of aspect 46, wherein the neural network comprises one or more convolutional layers.
48. The method of aspects 46 or 47, wherein the neural network is bidirectional.
49. The method of aspect 48, wherein the neural network comprises a transformer encoder.
50. The method of aspect 49, wherein the machine learning model is a Hidden-Unit Bidirectional Encoder Representations from Transformers (HuBERT) model.
51. The method of any of aspects 45-50, wherein the set of discrete speech units are generated by training the encoding machine learning model.
52. The method of aspect 51, wherein the training is self-supervised training.
53. The method of any of aspects 45-52, wherein the method further comprises:
   obtaining reference electronic speech waveforms for a plurality of phrases;
   encoding each reference speech waveform into a temporal sequence of discrete speech units using the encoding model;
   recording the brain electrical signal data associated with attempted speech by the subject for each one of the plurality of phrases;
   training the decoding machine learning model to predict the most likely discrete speech unit associated with a segment of electrical signal data using the speech units derived from the reference speech waveforms and the brain electrical signal data associated with attempted speech for each one of the plurality of phrases.
54. The method of aspect 53, wherein the reference speech waveforms are obtained from a recruited speaker.
55. The method of aspect 53, wherein the reference speech waveforms are obtained using a text-to-speech algorithm.
56. The method of any of aspects 53-55, wherein the decoding machine learning model is trained to learn mappings between neural activity patterns of electrical signals in the brain electrical signal data and the discrete speech units.
57. The method of any of aspects 53-56, wherein the training uses a CTC loss function.
58. The method of any of aspects 39-57, wherein each speech sound is decoded from one or more discrete speech units using a speech synthesizer.
59. The method of aspect 58, wherein the speech synthesizer comprises a machine learning model.
60. The method of aspect 59, wherein the synthesizing machine learning model comprises a neural network.
61. The method of aspect 60, wherein the neural network comprises one or more convolutional layers.
62. The method of aspects 60 or 61, wherein the neural network is bidirectional.
63. The method of aspect 62, wherein the neural network is an RNN.
64. The method of aspect 63, wherein the RNN comprises GRUs or LSTM.
65. The method of aspect 64, wherein the RNN comprises one or more LSTM layers.
66. The method of any of aspects 60-65, wherein the neural network comprises an attention mechanism.
67. The method of any of aspects 59-66, wherein the synthesizing machine learning model is configured to generate a spectrogram from the one or more discrete speech units.
68. The method of aspect 67, wherein the spectrogram is a mel spectrogram.
69. The method of aspects 67 or 68, wherein the speech synthesizer further comprises a vocoder configured to synthesize an electronic speech waveform from the spectrogram.
70. The method of aspect 69, wherein the vocoder comprises a machine learning model.
71. The method of aspect 70, wherein the vocoder machine learning model comprises a neural network.
72. The method of aspect 71, wherein the neural network is an RNN.
73. The method of any of aspects 69-72, wherein the method further comprises transforming the electronic speech waveform into a personalized electronic speech waveform.
74. The method of aspect 73, wherein the personalized electronic speech waveform resembles speech in the subject's own voice.
75. The method of aspects 73 or 74, wherein the transforming is performed using a machine learning model.
76. The method of aspect 75, wherein the transforming machine learning model comprises a neural network.
77. The method of aspect 76, wherein the neural network is based on transformer architecture.
78. The method of any of aspects 69-77, wherein the method further comprises converting the electronic speech waveform into an audible speech waveform.
79. The method of aspect 78, wherein the electronic speech waveform is converted into an audible speech waveform using a loudspeaker.
80. The method of any of the preceding aspects, wherein the processor is provided by a computer or handheld device.
81. The method of aspect 80, wherein the handheld device is a cell phone or a tablet.
82. A non-transitory computer-readable medium comprising program instructions that, when executed by a processor, cause the processor to perform the method of any one of aspects 1-81.
83. A kit comprising the non-transitory computer-readable medium of aspect 82 and instructions for decoding brain electrical signal data associated with attempted speech by a subject.
84. A system for decoding speech sounds from recorded brain electrical signal data configured to perform the method according to any of Aspects 1-81.
85. A computer implemented method for decoding speech audio from recorded brain electrical signal data associated with attempted speech by a subject, the computer performing steps comprising:
   receiving the recorded brain electrical signal data associated with the attempted speech by the subject; and
   decoding one or more speech sounds from the recorded brain electrical signal data using a machine learning model.
86. The computer implemented method of aspect 85, wherein the decoding machine learning model comprises a neural network.
87. The computer implemented method of aspect 86, wherein the neural network comprises one or more convolutional layers.
88. The computer implemented method of aspects 86 or 87, wherein the neural network is bidirectional.
89. The computer implemented method of aspect 88, wherein the neural network is a recurrent neural network (RNN).
90. The computer implemented method of aspect 89, wherein the RNN comprises gated recurrent units (GRUs) or long-short term memory (LSTM).
91. The computer implemented method of aspect 90, wherein the RNN comprises GRUs.
92. The computer implemented method of any of aspects 85-91, wherein one or more discrete speech units of a set of discrete speech units are decoded from the recorded brain electrical signal data.
93. The computer implemented method of aspect 92, wherein each speech sound is decoded from one or more discrete speech units.
94. The computer implemented method of aspects 92 or 93, wherein the set of discrete speech units comprises 50 or more discrete speech units.
95. The computer implemented method of aspect 94, wherein the set of discrete speech units comprises 100 or more discrete speech units.
96. The computer implemented method of any of aspects 92-95, wherein discrete speech units are continuously decoded at a uniform frequency.
97. The computer implemented method of aspect 96, wherein the frequency is 50 Hz or more.
98. The computer implemented method of aspect 97, wherein the frequency is 200 Hz or more.
99. The computer implemented method of any of aspects 92-98, wherein the set of discrete speech units are generated using an encoding machine learning model.
100. The computer implemented method of aspect 99, wherein the encoding machine learning model comprises a neural network.
101. The computer implemented method of aspect 100, wherein the neural network comprises one or more convolutional layers.
102. The computer implemented method of aspects 100 or 101, wherein the neural network is bidirectional.
103. The computer implemented method of aspect 102, wherein the neural network comprises a transformer encoder.
104. The computer implemented method of aspect 103, wherein the machine learning model is a Hidden-Unit Bidirectional Encoder Representations from Transformers (HuBERT) model.
105. The computer implemented method of any of aspects 99-104, wherein the set of discrete speech units are generated by training the encoding machine learning model.
106. The computer implemented method of aspect 105, wherein the training is self-supervised training.
107. The computer implemented method of any of aspects 99-106, wherein the method further comprises:
   receiving reference electronic speech waveforms for a plurality of phrases;
   encoding each reference speech waveform into a temporal sequence of discrete speech units using the encoding model;
   receiving brain electrical signal data associated with attempted speech by the subject for each one of the plurality of phrases;
   training the decoding machine learning model to predict the most likely discrete speech unit associated with a segment of electrical signal data using the speech units derived from the reference speech waveforms and the brain electrical signal data associated with attempted speech for each one of the plurality of phrases.
108. The computer implemented method of aspect 107, wherein the reference speech waveforms are generated using a text-to-speech algorithm.
109. The computer implemented method of aspects 107 or 108, wherein the decoding machine learning model is trained to learn mappings between neural activity patterns of electrical signals in the brain electrical signal data and the discrete speech units.
110. The computer implemented method of any of aspects 93-109, wherein each speech sound is decoded from one or more discrete speech units using a speech synthesizer.
111. The computer implemented method of aspect 110, wherein the speech synthesizer comprises a machine learning model.
112. The computer implemented method of aspect 111, wherein the synthesizing machine learning model comprises a neural network.
113. The computer implemented method of aspect 112, wherein the neural network comprises one or more convolutional layers.
114. The computer implemented method of aspects 112 or 113, wherein the neural network is bidirectional.
115. The computer implemented method of aspect 114, wherein the neural network is an RNN.
116. The computer implemented method of aspect 115, wherein the RNN comprises GRUs or LSTM.
117. The computer implemented method of aspect 116, wherein the RNN comprises one or more LSTM layers.
118. The computer implemented method of any of aspects 112-117, wherein the neural network comprises an attention mechanism.
119. The computer implemented method of any of aspects 111-118, wherein the synthesizing machine learning model is configured to generate a spectrogram from the one or more discrete speech units.
120. The computer implemented method of aspect 119, wherein the spectrogram is a mel spectrogram.
121. The computer implemented method of aspects 119 or 120, wherein the speech synthesizer further comprises a vocoder configured to synthesize an electronic speech waveform from the spectrogram.
122. The computer implemented method of aspect 121, wherein the vocoder comprises a machine learning model.
123. The computer implemented method of aspect 122, wherein the vocoder machine learning model comprises a neural network.
124. The computer implemented method of aspect 123, wherein the neural network is an RNN.
125. The computer implemented method of any of aspects 121-124, wherein the method further comprises transforming the electronic speech waveform into a personalized electronic speech waveform.
126. The computer implemented method of aspect 125, wherein the personalized electronic speech waveform resembles speech in the subject's own voice.
127. The computer implemented method of aspects 125 or 126, wherein the transforming is performed using a machine learning model.
128. The computer implemented method of aspect 127, wherein the transforming machine learning model comprises a neural network.
129. The computer implemented method of aspect 128, wherein the neural network is based on transformer architecture.
131. A non-transitory computer-readable medium comprising program instructions that, when executed by a processor, cause the processor to perform the computer implemented method of any one of aspects 85-129.
132. A kit comprising the non-transitory computer-readable medium of aspect 131 and instructions for decoding brain electrical signal data associated with attempted speech by a subject.
133. A system for producing speech audio directly from neural activity, the system comprising:
   a neural recording device comprising an electrode adapted for positioning at a location in a sensorimotor cortex region of the brain of the subject to record brain electrical signal data associated with attempted speech by the subject;
   a processor programmed to use a machine learning model to decode one or more speech sounds from the recorded brain electrical signal data;
   an interface in communication with a computing device, said interface adapted for positioning at a location on the head of the subject, wherein the interface receives the brain electrical signal data from the neural recording device and transmits the brain electrical signal data to the processor; and
   an audio speaker for playing the one or more speech sounds from the recorded brain electrical signal data.
134. The system of aspect 133, wherein the subject has difficulty with said speech because of anarthria, a stroke, a traumatic brain injury, a brain tumor, or amyotrophic lateral sclerosis.
135. The system of any of aspects 133-134, wherein the neural recording device is adapted for positioning on a surface of the sensorimotor cortex region or within the sensorimotor cortex region.
136. The system of any of aspects 133-135, wherein the electrode of the neural recording device is adapted for positioning on the pial surface of the sensorimotor cortex.
137. The system of any of aspects 133-136, wherein the neural recording device is adapted to be positioned such that the recording device covers regions associated with speech production and language perception.
138. The system of aspect 137, wherein the covered regions include a middle portion of the superior and/or middle temporal gyrus, the precentral gyrus, and/or the postcentral gyrus.
139. The system of any of aspects 133-138, wherein the neural recording device is adapted to be centered on the central sulcus.
140. The system of any of aspects 133-139, wherein the neural recording device comprises an electrocorticography (ECoG) electrode array.
141. The system of aspect 140, wherein the ECoG electrode array is a high-density array.
142. The system of aspect 141, wherein the high-density array comprises 200 electrodes or more.
143. The system of any of aspects 140-142, wherein the electrode array comprises non-penetrating surface electrodes.
144. The system of any of aspects 133-143, wherein the interface comprises a percutaneous pedestal connector attached to the subject's cranium.
145. The system of any of aspects 133-144, wherein the interface further comprises a headstage connected to the percutaneous pedestal connector.
146. The system of aspect 145, wherein the headstage is configured to process and digitize the brain electrical signal data before transmitting the data to the processor.
147. The system of any of aspects 133-146, wherein the electrical signal data comprises high-gamma frequency content features.
148. The system of any of aspects 133-147, wherein the electrical signal data comprises neural oscillations in a range from 70 Hz to 150 Hz.
149. The system of any of aspects 133-148, wherein the electrical signal data comprises low-frequency signals.
150. The system of aspect 149, wherein the electrical signal data comprises neural oscillations in a range from 0.3 Hz to 17 Hz.
151. The system of any of aspects 133-150, wherein the brain electrical signal data is recorded from a sensorimotor cortex region selected from the precentral gyrus, postcentral gyrus, superior temporal gyrus, middle temporal gyrus, or any combination thereof.
153. The system of any of aspects 133-152, wherein the one or more speech sounds form a word.
154. The system of aspect 153, wherein the one or more speech sounds form a sentence.
155. The system of aspects 153 or 154, wherein the subject is limited to a specified word set for the attempted speech.
156. The system of aspect 155, wherein the word set comprises words for expressing basic concepts and/or caregiving needs.
157. The system of aspects 155 or 156, wherein the word set comprises 100 words or more.
158. The system of aspect 157, wherein the word set comprises 350 words or more.
159. The system of aspect 158, wherein the word set comprises 1000 words or more.
160. The system of any of aspects 155-159, wherein the subject is limited to two or more word sets for the attempted speech.
161. The system of aspect 160, wherein the processor is configured to switch between word sets.
162. The system of any of aspects 133-161, wherein the decoding machine learning model comprises a neural network.
163. The system of aspect 162, wherein the neural network comprises one or more convolutional layers.
164. The system of aspects 162 or 163, wherein the neural network is bidirectional.
165. The system of aspect 164, wherein the neural network is a recurrent neural network (RNN).
166. The system of aspect 165, wherein the RNN comprises gated recurrent units (GRUs) or long-short term memory (LSTM).
167. The system of aspect 166, wherein the RNN comprises GRUs.
168. The system of any of aspects 133-167, wherein one or more discrete speech units of a set of discrete speech units are decoded from the recorded brain electrical signal data.
169. The system of aspect 168, wherein each speech sound is decoded from one or more discrete speech units.
170. The system of any of aspects 168-169, wherein discrete speech units are continuously decoded at a uniform frequency.
171. The system of aspect 170, wherein the frequency is 200 Hz or more.
172. The system of any of aspects 168-171, wherein the set of discrete speech units are generated using an encoding machine learning model.
173. The system of aspect 172, wherein the encoding machine learning model comprises a neural network.
174. The system of aspect 173, wherein the neural network comprises one or more convolutional layers.
175. The system of aspects 173 or 174, wherein the neural network is bidirectional.
176. The system of aspect 175, wherein the neural network comprises a transformer encoder.
177. The system of aspect 176, wherein the machine learning model is a Hidden-Unit Bidirectional Encoder Representations from Transformers (HuBERT) model.
178. The system of any of aspects 172-177, wherein the set of discrete speech units are generated by training the encoding machine learning model.
179. The system of aspect 178, wherein the training is self-supervised training.
180. The system of any of aspects 172-179, wherein the processor is further programmed to:
   obtain reference electronic speech waveforms for a plurality of phrases;
   encode each reference speech waveform into a temporal sequence of discrete speech units using the encoding model;
   record the brain electrical signal data associated with attempted speech by the subject for each one of the plurality of phrases;
   train the decoding machine learning model to predict the most likely discrete speech unit associated with a segment of electrical signal data using the speech units derived from the reference speech waveforms and the brain electrical signal data associated with attempted speech for each one of the plurality of phrases.
181. The system of aspect 180, wherein the system further comprises a microphone configured to generate reference speech waveforms from a recruited speaker.
182. The system of aspect 180, wherein the processor further comprises a text-to-speech algorithm configured to generate the reference speech waveforms.
183. The system of any of aspects 180-182, wherein the decoding machine learning model is trained to learn mappings between neural activity patterns of electrical signals in the brain electrical signal data and the discrete speech units.
184. The system of any of aspects 172 to 183, wherein each speech sound is decoded from one or more discrete speech units using a speech synthesizer.
185. The system of aspect 184, wherein the speech synthesizer comprises a machine learning model.
186. The system of aspect 185, wherein the synthesizing machine learning model comprises a neural network.
187. The system of aspect 186, wherein the neural network comprises one or more convolutional layers.
188. The system of aspects 186 or 187, wherein the neural network is bidirectional.
189. The system of aspect 188, wherein the neural network is an RNN.
190. The system of aspect 189, wherein the RNN comprises GRUs or LSTM.
191. The system of aspect 190, wherein the RNN comprises one or more LSTM layers.
192. The system of any of aspects 186 to 191, wherein the neural network comprises an attention mechanism.
193. The system of any of aspects 185 to 192, wherein the synthesizing machine learning model is configured to generate a spectrogram from the one or more discrete speech units.
194. The system of aspect 193, wherein the spectrogram is a mel spectrogram.
195. The system of aspects 193 or 194, wherein the speech synthesizer further comprises a vocoder configured to synthesize an electronic speech waveform from the spectrogram.
196. The system of aspect 195, wherein the vocoder comprises a machine learning model.
197. The system of aspect 196, wherein the vocoder machine learning model comprises a neural network.
198. The system of aspect 197, wherein the neural network is an RNN.
199. The system of any of aspects 195 to 198, wherein the processor is further programmed to transform the electronic speech waveform into a personalized electronic speech waveform.
200. The system of aspect 199, wherein the personalized electronic speech waveform resembles speech in the subject's own voice.
201. The system of aspects 199 or 200, wherein the transforming is performed using a machine learning model.
202. The system of aspect 201, wherein the transforming machine learning model comprises a neural network.
203. The system of aspect 202, wherein the neural network is based on transformer architecture.
204. The system of any of aspects 195-203, wherein the processor is further programmed to convert the electronic speech waveform into an audible speech waveform.
205. The system of aspect 204, wherein the electronic speech waveform is converted into an audible speech waveform using a loudspeaker.
206. The system of any of aspects 133-205, wherein the processor is provided by a computer or handheld device.
207. The system of aspect 206, wherein the handheld device is a cell phone or a tablet.
208. A kit comprising the system of any of aspects 133-207 and instructions for decoding brain electrical signal data associated with attempted speech by a subject.
209. A method of controlling an electronic output device or system to perform one or more actions using brain electrical signals, the method comprising:
   positioning a neural recording device comprising an electrode at a location in a sensorimotor cortex region of the brain of the subject to record brain electrical signal data associated with an attempted action by the subject;
   positioning an interface in communication with a computing device at a location on the head of the subject, wherein the interface is connected to the neural recording device;
   recording the brain electrical signal data associated with the attempted action by the subject using the neural recording device, wherein the interface receives the brain electrical signal data from the neural recording device and transmits the brain electrical signal data to a processor of the computing device;
   decoding one or more electronic output device or system actions from the recorded brain electrical signal data, wherein the processor is programmed to use a machine learning model for the decoding; and
   controlling the electronic output device or system to perform the one or more decoded electronic output device or system actions.
210. The method of aspect 209, wherein the subject has difficulty communicating because of anarthria, a stroke, a traumatic brain injury, a brain tumor, or amyotrophic lateral sclerosis.
211. The method of aspect 209 or 210, wherein the subject is paralyzed.
212. The method of any of aspects 209-211, wherein the subject is quadriplegic and/or experiences partial or total facial paralysis.
213. The method of any of aspects 209-212, wherein the location of the neural recording device is on a surface of the sensorimotor cortex region or within the sensorimotor cortex region.
214. The method of any of aspects 209-213, wherein the electrode of the neural recording device is positioned on the pial surface of the sensorimotor cortex.
215. The method of any of aspects 209-214, wherein the neural recording device is positioned such that the recording device covers regions associated with speech production and language perception.
216. The method of aspect 215, wherein the covered regions include a middle portion of the superior and/or middle temporal gyrus, the precentral gyrus, and/or the postcentral gyrus. 217. The method of any of aspects 209-216, wherein the neural recording device is centered on the central sulcus.
218. The method of any of aspects 209-217, wherein the neural recording device comprises an electrocorticography (ECoG) electrode array.
219. The method of aspect 218, wherein the ECoG electrode array is a high-density array.
220. The method of aspect 219, wherein the high-density array comprises 200 electrodes or more.
221. The method of any of aspects 218-220, wherein the electrode array comprises non-penetrating surface electrodes.
222. The method of any of aspects 209-221, wherein the interface comprises a percutaneous pedestal connector attached to the subject's cranium.
223. The method of any of aspects 209-222, wherein the interface further comprises a headstage connected to the percutaneous pedestal connector.
224. The method of aspect 223, wherein the headstage processes and digitizes the brain electrical signal data before transmitting the data to the processor.
225. The method of any of aspects 209-224, wherein the electrical signal data comprises high-gamma frequency content features.
226. The method of any of aspects 209-225, wherein the electrical signal data comprises neural oscillations in a range from 70 Hz to 150 Hz.
227. The method of any of aspects 209-226, wherein the electrical signal data comprises low-frequency signals.
228. The method of aspect 227, wherein the electrical signal data comprises neural oscillations in a range from 0.3 Hz to 17 Hz.
229. The method of any of aspects 209-228, wherein the brain electrical signal data is recorded from a sensorimotor cortex region selected from the precentral gyrus, postcentral gyrus, superior temporal gyrus, middle temporal gyrus, or any combination thereof.
230. The method of any of aspects 209-229, wherein the method further comprises mapping the brain of the subject to identify an optimal location for positioning the electrode for recording the brain electrical signals associated with the attempted speech by the subject.
231. The method of any of aspects 209-230, wherein the subject is limited to a specified action set for the attempted action.
232. The method of aspect 231, wherein the action set comprises actions for expressing basic emotions and/or communicating caregiving needs.
233. The method of aspects 231 or 232, wherein the action set comprises 6 actions or more.
234. The method of aspect 233, wherein the action set comprises 100 actions or more.
235. The method of aspect 234, wherein the action set comprises 500 actions or more.
236. The method of any of aspects 231-235, wherein the subject is limited to two or more action sets for the attempted action.
237. The method of aspect 28236 wherein the subject may switch between action sets.
238. The method of any of aspects 209-237, wherein the decoding machine learning model comprises a neural network.
239. The method of aspect 238, wherein the neural network comprises one or more convolutional layers.
240. The method of aspects 238 or 239, wherein the neural network is bidirectional.
241. The method of aspect 240, wherein the neural network is a recurrent neural network (RNN).
242. The method of aspect 241, wherein the RNN comprises gated recurrent units (GRUs) or long-short term memory (LSTM).
243. The method of aspect 242, wherein the RNN comprises GRUs.
244. The method of any of aspects 209-243, wherein each action of the device or system is discretized into one or more action representations.
245. The method of aspect 244, wherein the action representations are decoded from the recorded brain electrical signal data.
246. The method of aspect 245, wherein each action of the device or system is decoded from one or more action representations.
247. The method of aspect 246, wherein discrete action representations are continuously decoded from the recorded brain electrical signal data at a uniform frequency.
248. The method of aspect 247, wherein the frequency is 50 Hz or more.
249. The method of aspect 248, wherein the frequency is 200 Hz or more.
250. The method of any of aspects 244-249, wherein the discretization is performed using an autoencoder.
251. The method of aspect 250, wherein the autoencoder comprises one or more convolutional layers.
252. The method of aspects 250 or 251, wherein the autoencoder uses one or more rectified linear unit (ReLU) activations.
253. The method of aspect 252, wherein the autoencoder comprises a vector-quantized variational autoencoder (VQ-VAE).
254. The method of any of aspects 250-253, wherein the discretization occurs during training of the autoencoder.
255. The method of aspect 254, wherein the training is self-supervised training.
256. The method of any of aspects 250-255, wherein the attempted action performed by the subject is different than the one or more electronic output device or system actions.
257. The method of aspect 256, wherein the attempted action performed by the subject is a hand gesture and the action of the electronic output device or system is powering on or off.
258. The method of any of aspects 250-255, wherein the attempted action performed by the subject corresponds to the one or more electronic output device or system actions.
259. The method of aspect 258, wherein the electronic output device or system is a prosthetic limb.
260. The method of aspect 258, wherein the electronic output device or system comprises a visual display and/or a loudspeaker.
261. The method of aspect 260, wherein the visual display and/or loudspeaker is configured to present a humanoid avatar.
262. The method of aspect 261, wherein the one or more electronic output device or system actions comprise actions performed by the avatar.
263. The method of aspect 262, wherein actions performed by the avatar include speech, orofacial movements for speech, and/or non-speech communicative gestures.
264. The method of aspect 263, wherein the speech orofacial movements comprise one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction.
265. The method of aspect 263, wherein the non-speech communicative gestures comprise the abduction, adduction, flexion, extension, and/or circumduction of one or more body parts.
266. The method of aspects 263 or 265, wherein the non-speech communicative gestures include emotional expressions using facial muscles.
267. The method of aspect 266, wherein the emotional expressions comprise happy, sad, and surprised expressions.
268. The method of any of aspects 262-267, wherein the method further comprises:
   obtaining reference avatar animations for a plurality of actions;
   encoding each reference animation into a temporal sequence of discrete action representations using the autoencoder;
   recording the brain electrical signal data associated with attempted action by the subject for each one of the plurality of actions;
   training the decoding machine learning model to predict the most likely discrete action representation associated with a segment of electrical signal data using the action representations derived from the reference avatar animations and the brain electrical signal data associated with attempted action for each one of the plurality of actions.
269. The method of aspect 268, wherein the reference avatar animations are obtained from an avatar-animation system.
270. The method of any of aspects 268 or 268, wherein the decoding machine learning model is trained to learn mappings between neural activity patterns of electrical signals in the brain electrical signal data and the discrete action representations.
271. The method of any of aspects 268-270, wherein the training uses a CTC loss function.
272. The method of any of aspects 250-271, wherein each action of the avatar is decoded from one or more action representations using the autoencoder.
273. The method of any of aspects 268-272, wherein the plurality of actions includes at least two of speech, orofacial movements for speech, and/or non-speech communicative gestures.
274. The method of aspect 273, wherein the decoding machine learning model is trained to discriminate between actions performed by different regions of the body and/or between speech associated actions and non-speech communicative gestures.
275. The method of aspect 274, wherein the decoding machine learning model is trained to discriminate between finger-flexions and actions associated with attempted speech.
276. The method of any of aspects 268-275, wherein the machine learning model is trained to decode orofacial movements for speech using action representations derived from reference avatar animations of the avatar performing the orofacial movements and brain electrical signal data associated with orofacial movements attempted by the subject.
277. The method of any of aspects 268-275, wherein the avatar is controlled to perform orofacial movements based on speech decoded from the recorded brain electrical signal data.
278. The method of aspect 277, wherein the avatar is controlled to perform orofacial movements using a speech-to-gesture algorithm.
279. The method of any of aspects 268-277, wherein a single decoding machine learning model is trained for speech, orofacial speech movement, and non-speech communicative gesture actions.
280. The method of any of aspects 268-277, wherein separate decoding machine learning models are trained for speech associated actions and non-speech communicative gestures.
281. The method of any of aspects 268-277 and 280, wherein separate decoding machine learning models are trained for speech and orofacial movements for speech.
282. The method of aspects 280 or 281, wherein the avatar is controlled using actions decoded from multiple machine learning models.
283. The method of aspect 282, wherein the actions decoded from the multiple machine learning models occur concurrently and the avatar is controlled to perform the actions simultaneously.
284. The method of any of aspects 273-283, wherein a decoded non-speech communicative gesture action affects how the avatar performs a decoded speech associated action. 285. The method of aspect 284, wherein a decoded emotional expression affects the inflection of decoded speech performed by the avatar.
286. The method of any of aspects 262-285, wherein the avatar is used by the subject to communicate with one or more individuals in person.
287. The method of any of aspects 262-286, wherein the avatar is used by the subject to communicate with one or more individuals in an interactive virtual environment.
288. The method of any of aspects 262-287, wherein the avatar is used by the subject to play a video game.
289. The method of any of aspects 262-288, wherein the avatar is used by the subject for therapy.
290. The method of aspect 289, wherein the avatar is used by the subject for physical therapy.
291. The method of aspect 290, wherein the physical therapy involves regaining mobility of a body part after an injury.
292. The method of any of aspects 262-291, wherein actions performed by the avatar control one or more electronic devices in the subject's environment.
293. The method of aspect 292, wherein actions performed by the avatar control one or more electronic devices in the same room as the subject.
294. The method of aspect 293, wherein actions performed by the avatar control one or smart home appliances.
295. The method of any of aspects 262-294, wherein the visual display comprises a computer monitor, a television, and/or a visual projection device.
296. The method of any of aspects 262-295, wherein the visual display comprises a virtual reality headset, goggles, or contacts.
297. The method of any of aspects 262-295, wherein the visual display comprises an augmented reality headset, goggles, or contacts.
298. The method of any of aspects 262-297, wherein the method further comprises generating a virtual environment for the avatar.
299. The method of aspect 298, wherein the action performed by the avatar is determined using the virtual environment.
300. The method of any of aspects 262-299, wherein previous actions performed by the avatar are used to determine the action performed by the avatar.
301. The method of any of aspects 209-300, wherein the processor is provided by a computer, a handheld device, or a headset.
302. The method of aspect 301, wherein the handheld device is a cell phone or a tablet.
303. A non-transitory computer-readable medium comprising program instructions that, when executed by a processor, cause the processor to perform the method of any one of aspects 209-302.
304. A kit comprising the non-transitory computer-readable medium of aspect 303 and instructions for decoding brain electrical signal data associated with attempted speech by a subject.
305. A system for controlling an electronic device or system using brain electrical signals configured to perform the method according to any of Aspects 209-302.
306. A computer implemented method for controlling an electronic output device or system to perform one or more actions using brain electrical signals, the computer performing steps comprising:
   receiving the recorded brain electrical signal data associated with the attempted action by the subject; and
   decoding one or more electronic output device or system actions from the recorded brain electrical signal data using a machine learning model.
307. The computer implemented method of aspect 306, wherein the decoding machine learning model comprises a neural network.
308. The computer implemented method of aspect 307, wherein the neural network comprises one or more convolutional layers.
309. The computer implemented method of aspects 307 or 308, wherein the neural network is bidirectional.
310. The computer implemented method of aspect 309, wherein the neural network is a recurrent neural network (RNN).
311. The computer implemented method of aspect 310, wherein the RNN comprises gated recurrent units (GRUs) or long-short term memory (LSTM).
312. The computer implemented method of aspect 311, wherein the RNN comprises GRUs.
313. The computer implemented method of any of aspects 306-312, wherein the subject is limited to a specified action set for the attempted action.
314. The computer implemented method of aspect 313, wherein the action set comprises actions for expressing basic emotions and/or communicating caregiving needs.
315. The computer implemented method of aspect 314, wherein the action set comprises 6 actions or more.
316. The computer implemented method of aspect 315, wherein the action set comprises 100 actions or more.
317. The computer implemented method of aspect 316, wherein the action set comprises 500 actions or more.
318. The computer implemented method of any of aspects 313-317, wherein the subject is limited to two or more action sets for the attempted action.
319. The computer implemented method of aspect 318, wherein the subject may switch between action sets.
320. The computer implemented method of any of aspects 306-319, wherein each action of the device or system is discretized into one or more action representations.
321. The computer implemented method of aspect 320, wherein the action representations are decoded from the recorded brain electrical signal data.
322. The computer implemented method of aspect 321, wherein each action of the output device or system is decoded from one or more action representations.
323. The computer implemented method of any of aspects 320-322, wherein the discretization is performed using an autoencoder.
324. The computer implemented method of aspect 323, wherein the autoencoder comprises one or more convolutional layers.
325. The computer implemented method of aspects 323 or 324, wherein the autoencoder uses one or more rectified linear unit (ReLU) activations.
326. The computer implemented method of aspect 325, wherein the autoencoder comprises a vector-quantized variational autoencoder (VQ-VAE).
327. The computer implemented method of any of aspects 323-326, wherein the discretization occurs during training of the autoencoder.
328. The computer implemented method of aspect 327, wherein the training is self-supervised training.
329. The computer implemented method of any of aspects 306-328, wherein the electronic output device or system comprises a visual display and/or a loudspeaker.
330. The computer implemented method of aspect 329, wherein the visual display and/or loudspeaker is configured to present a humanoid avatar.
331. The computer implemented method of aspect 330, wherein the one or more electronic output device or system actions comprise actions performed by the avatar.
332. The computer implemented method of aspect 331, wherein actions performed by the avatar include speech, orofacial movements for speech, and/or non-speech communicative gestures.
333. The method of aspect 332, wherein the non-speech communicative gestures include emotional expressions using facial muscles.
334. The computer implemented method of any of aspects 331-333, wherein the method further comprises:
   receiving reference avatar animations for a plurality of actions;
   encoding each reference avatar animation into a temporal sequence of discrete action representations using the autoencoder;
   receiving brain electrical signal data associated with the attempted action by the subject for each one of the plurality of actions;
   training the decoding machine learning model to predict the most likely discrete action representation associated with a segment of electrical signal data using the action representations derived from the reference avatar animations and the brain electrical signal data associated with the attempted action for each one of the plurality of actions.
335. The computer implemented method of aspect 334, wherein separate decoding machine learning models are trained for speech associated actions and non-speech communicative gestures.
336. The computer implemented method of aspects 334 or 335, wherein the avatar is used by the subject to communicate with one or more individuals in person.
337. The computer implemented method of any of aspects 334-336, wherein the avatar is used by the subject to communicate with one or more individuals in an interactive virtual environment.
338. The computer implemented method of aspect 337, wherein the method further comprises generating a virtual environment for the avatar.
339. The computer implemented method of aspect 338, wherein the action performed by the avatar is determined using the virtual environment.
340. The computer implemented method of any of aspects 331-339, wherein previous actions performed by the avatar are used to determine the action performed by the avatar.
341. A non-transitory computer-readable medium comprising program instructions that, when executed by a processor, cause the processor to perform the computer implemented method of any one of aspects 306-340.
342. A kit comprising the non-transitory computer-readable medium of aspect 341 and instructions for decoding brain electrical signal data associated with attempted speech by a subject.
343. A system for controlling an avatar to perform one or more actions using brain electrical signals, the system comprising:
   a neural recording device comprising an electrode adapted for positioning at a location in a sensorimotor cortex region of the brain of the subject to record brain electrical signal data associated with an attempted action by the subject;
   a processor programmed to use a machine learning model to decode an avatar animation from the recorded brain electrical signal data;
   an interface in communication with a computing device, said interface adapted for positioning at a location on the head of the subject, wherein the interface receives the brain electrical signal data from the neural recording device and transmits the brain electrical signal data to the processor; and
   a display component for displaying the avatar action from the recorded brain electrical signal data.
344. The system of aspect 343, wherein the subject has difficulty with said speech because of anarthria, a stroke, a traumatic brain injury, a brain tumor, or amyotrophic lateral sclerosis.
345. The system of aspects 343 or 344, wherein the neural recording device is adapted for positioning on a surface of the sensorimotor cortex region or within the sensorimotor cortex region.
346. The system of any of aspects 343-345, wherein the electrode of the neural recording device is adapted for positioning on the pial surface of the sensorimotor cortex.
347. The system of any of aspects 343-345, wherein the neural recording device is adapted to be positioned such that the recording device covers regions associated with speech production and language perception.
348. The system of aspect 347, wherein the covered regions include a middle portion of the superior and/or middle temporal gyrus, the precentral gyrus, and/or the postcentral gyrus.
349. The system of any of aspects 343-348, wherein the neural recording device is adapted to be centered on the central sulcus.
350. The system of any of aspects 343-349, wherein the neural recording device comprises an electrocorticography (ECoG) electrode array.
351. The system of aspect 350, wherein the ECoG electrode array is a high-density array.
352. The system of aspect 351, wherein the high-density array comprises 200 electrodes or more.
353. The system of any of aspects 350-352, wherein the electrode array comprises non-penetrating surface electrodes. 354. The system of any of aspects 343-353, wherein the interface comprises a percutaneous pedestal connector attached to the subject's cranium.
355. The system of any of aspects 343-354, wherein the interface further comprises a headstage connected to the percutanous pedestal connector.
356. The system of aspect 355, wherein the headstage is configured to process and digitize the brain electrical signal data before transmitting the data to the processor.
357. The system of any of aspects 343-356, wherein the electrical signal data comprises high-gamma frequency content features.
358. The system of any of aspects 343-357, wherein the electrical signal data comprises neural oscillations in a range from 70 Hz to 150 Hz.
359. The system of any of aspects 343-358, wherein the electrical signal data comprises low-frequency signals.
360. The system of aspect 359, wherein the electrical signal data comprises neural oscillations in a range from 0.3 Hz to 17 Hz.
361. The system of any of aspects 343-360, wherein the brain electrical signal data is recorded from a sensorimotor cortex region selected from the precentral gyrus, postcentral gyrus, superior temporal gyrus, middle temporal gyrus, or any combination thereof.
362. The system of any of aspects 343-361, wherein the subject is limited to a specified action set for the attempted action.
363. The system of aspect 362, wherein the action set comprises actions for expressing basic emotions and/or communicating caregiving needs.
364. The system of aspects 362 or 363, wherein the action set comprises 6 actions or more.
365. The system of aspect 364, wherein the action set comprises 100 actions or more.
366. The system of any of aspects 362-365, wherein the subject is limited to two or more action sets for the attempted action.
367. The system of aspect 366, wherein the processor is configured to switch between action sets.
368. The system of any of aspects 343-367, wherein the decoding machine learning model comprises a neural network.
369. The system of aspect 368 wherein the neural network comprises one or more convolutional layers.
370. The system of aspects 368 or 369, wherein the neural network is bidirectional.
371. The system of aspect 370, wherein the neural network is a recurrent neural network (RNN).
372. The system of aspect 371, wherein the RNN comprises gated recurrent units (GRUs) or long-short term memory (LSTM).
373. The system of aspect 372, wherein the RNN comprises GRUs.
374. The system of any of aspects 343-373, wherein the processor is further programmed to discretize each avatar action into one or more action representations.
375. The system of aspect 374, wherein the action representations are decoded from the recorded brain electrical signal data.
376. The system of aspect 375, wherein each avatar action is decoded from one or more action representations.
377. The system of any of aspects 374-376, wherein the discretization is performed using an autoencoder.
378. The system of aspect 377, wherein the autoencoder comprises one or more convolutional layers.
379. The system of aspects 377 or 378, wherein the autoencoder uses one or more rectified linear unit (ReLU) activations.
380. The system of aspect 379, wherein the autoencoder comprises a vector-quantized variational autoencoder (VQ-VAE).
381. The system of any of aspects 377-380, wherein the discretization occurs during training of the autoencoder.
382. The system of aspect 381, wherein the training is self-supervised training.
383. The system of any of aspects 343-382, wherein actions performed by the avatar include speech, orofacial movements for speech, and/or non-speech communicative gestures.
384. The system of aspect 383, wherein the speech orofacial movements comprise one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction.
385. The system of aspect 383, wherein the non-speech communicative gestures comprise the abduction, adduction, flexion, extension, and/or circumduction of one or more body parts.
386. The system of aspects 383 or 385, wherein the non-speech communicative gestures include emotional expressions using facial muscles.
387. The system of aspect 58, wherein the emotional expressions comprise happy, sad, and/or surprised expressions.
388. The system of any of aspects 377-387, wherein the processor is further programmed to:
   obtain reference avatar animations for a plurality of actions;
   encode each reference animation into a temporal sequence of discrete action representations using the autoencoder;
   record brain electrical signal data associated with the attempted action by the subject for each one of the plurality of actions;
   train the decoding machine learning model to predict the most likely discrete action representation associated with a segment of electrical signal data using the action representations derived from the reference avatar animations and the brain electrical signal data associated with attempted action for each one of the plurality of actions.
389. The system of aspect 388, wherein the decoding machine learning model is trained to learn mappings between neural activity patterns of electrical signals in the brain electrical signal data and the discrete action representations.
390. The system of aspects 388 or 389, wherein the training uses a CTC loss function.
391. The system of any of aspects 377-390, wherein each action of the avatar is decoded from one or more action representations using the autoencoder.
392. The system of any of aspects 388-391, wherein the plurality of actions includes at least two of speech, orofacial movements for speech, and/or non-speech communicative gestures.
393. The system of aspect 392, wherein the decoding machine learning model is trained to discriminate between actions performed by different regions of the body and/or between speech associated actions and non-speech communicative gestures.
394. The system of any of aspects 377-393, wherein separate decoding machine learning models are trained for speech associated actions and non-speech communicative gestures. 395. The system of aspect 394, wherein separate decoding machine learning models are trained for speech and orofacial movements for speech.
396. The system of aspects 394 or 395, wherein the avatar is controlled using actions decoded from multiple machine learning models.
397. The system of aspect 396, wherein the actions decoded from the multiple machine learning models occur concurrently and the avatar is controlled to perform the actions simultaneously.
398. The system of any of aspects 343-397, wherein the visual display comprises a computer monitor, a television, and/or a visual projection device.
399. The system of any of aspects 343-398, wherein the visual display comprises a virtual reality headset, goggles, or contacts.
400. The system of any of aspects 343-399, wherein the visual display comprises an augmented reality headset, goggles, or contacts.
401. The system of any of the aspects 343-400, wherein the processor is further programmed to generate a virtual environment for the avatar.
402. The system of aspect 401, wherein the action performed by the avatar is determined using the virtual environment.
403. The system of any of the aspects 343-402, wherein previous actions performed by the avatar are used to determine the action performed by the avatar.
404. The system of any of aspects 343-403, wherein the processor is provided by a computer, a handheld device, or a headset.
405. The system of aspect 404, wherein the handheld device is a cell phone or a tablet.
406. A kit comprising the system of any of aspects 343-405 and instructions for decoding brain electrical signal data associated with attempted speech by a subject.

### EXPERIMENTAL

As demonstrated in the above disclosure, the present invention has a wide variety of applications. The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, percentages, etc.) but some experimental errors and deviations should be accounted for.

### Example 1: A High-Performance Neuroprosthesis for Speech Decoding and Avatar Control

### 1.1. OVERVIEW

Speech neuroprostheses have the potential to restore communication to people living with paralysis, but naturalistic speed and expressivity remain elusive. Here, high-density cortical-surface recordings were used in a clinical-trial participant with severe limb and vocal paralysis to achieve high-performance real-time decoding across three complementary speech-related output modalities: text, speech audio, and facial-avatar animation. Deep-learning models were trained and evaluated using neural data collected as the participant attempted to silently speak sentences. For text, accurate and rapid large-vocabulary decoding was demonstrated with a median rate of 77.6 words per minute and median word error rate of 25.5%. For speech sounds, intelligible speech synthesis of high-utility phrases was demonstrated, with untrained listeners achieving a median perceptual word error rate of 29.2% during transcription. For facial avatar, the control of virtual orofacial movements for speech and non-speech communicative gestures was demonstrated. The decoders reached high performance with fewer than two weeks of training. The findings introduce a new multimodal speech-neuroprosthetic approach that has significant promise to restore full, embodied communication to people living with severe paralysis.

### 1.2. INTRODUCTION

Speech is the ability to express thoughts and ideas through spoken words. Speech loss after neurological injury is devastating because it significantly impairs communication and causes social isolation [1]. Previous demonstrations have shown that it is possible to decode speech from the brain activity of a person with paralysis, but only in the form of text and with limited speed and vocabulary [2] [3]. A compelling goal is to both enable faster large-vocabulary text-based communication and restore the produced speech sounds and facial movements related to speaking. While text outputs are good for basic communication and messaging, speaking has rich prosody, expressiveness, and identity that can enhance embodied communication beyond what can be conveyed in text alone. To address this, a multimodal speech neuroprosthesis that uses broad-coverage, high-density electrocorticography was designed to decode text and audio-visual speech outputs from articulatory vocal-tract representations distributed throughout the sensorimotor cortex. Due to severe paralysis caused by a brainstem stroke that occurred over 18 years ago, the participant cannot speak or vocalize speech sounds given the severe weakness of her orofacial muscles (anarthria) and cannot type given the weakness in her arms and hands (quadriplegia). Instead, she uses commercially available assistive technology to communicate, primarily relying on a head-tracking interface to generate intended messages at about 14 words per minute.

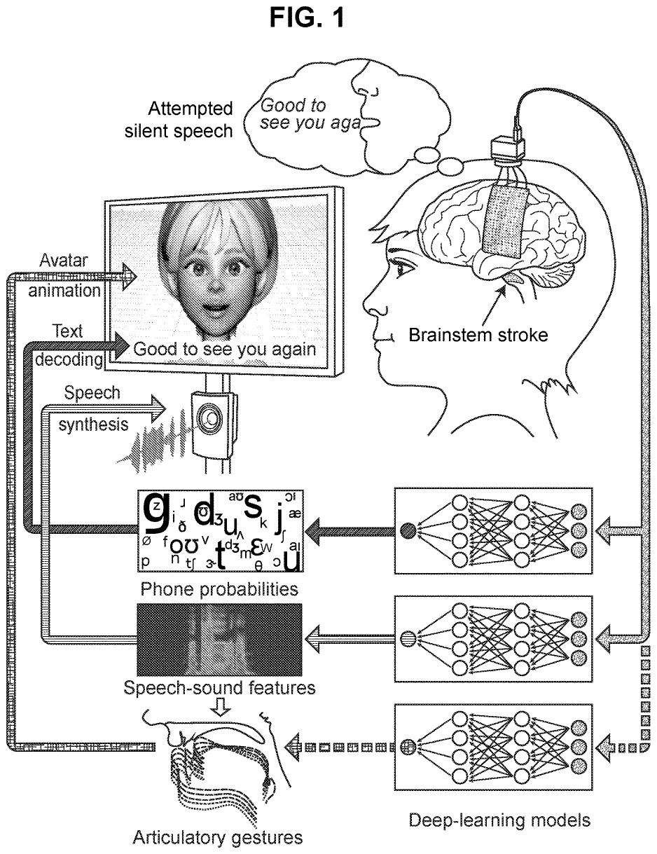

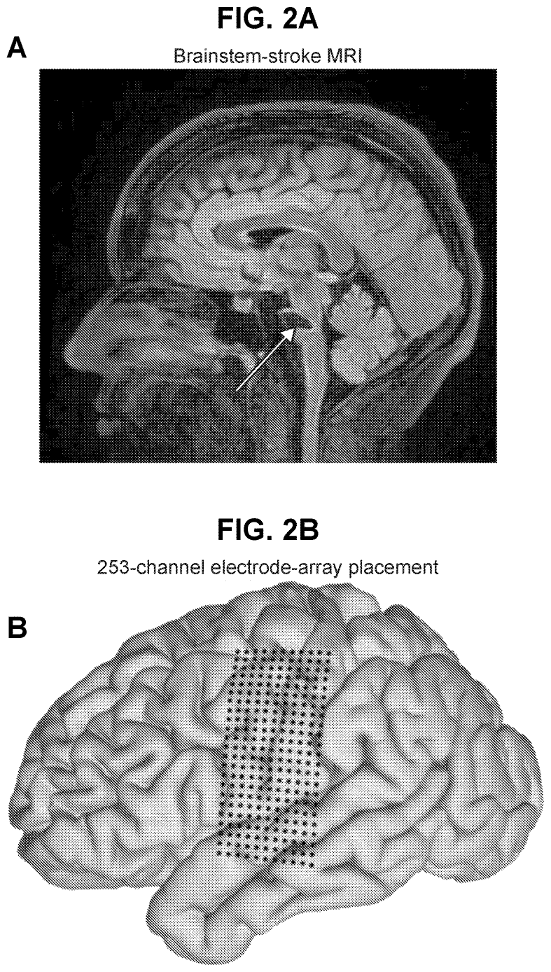

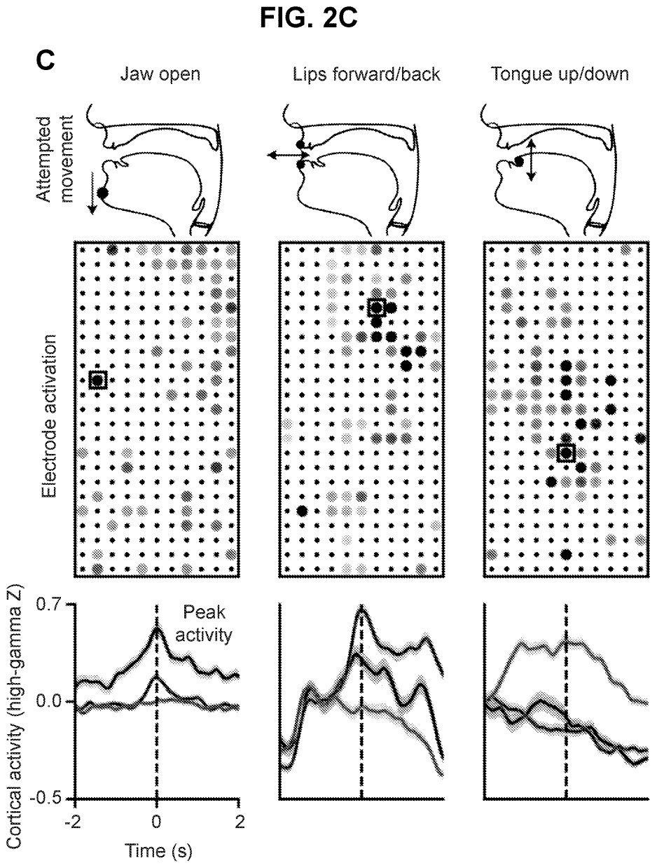

A speech-decoding system was designed that enabled a clinical-trial participant (ClinicalTrials.gov; NCT03698149) with severe paralysis and anarthria to communicate by decoding intended sentences from signals acquired by a 253-channel high-density electrocorticography (ECoG) array implanted over her sensorimotor cortex (FIG. 1 and FIGS. 2A to 2B). The array was positioned over cortical areas relevant for orofacial movements, and simple movement tasks demonstrated differentiable activations associated with attempted movements of the lips, tongue, and jaw (FIG. 2C).

For speech decoding, the participant was presented with a sentence as a text prompt on a screen and was instructed to silently attempt to say the sentence to the best of her ability after a visual go cue. Meanwhile, neural signals recorded from all 253 ECoG electrodes were processed to extract high-gamma activity (HGA; between 70-150 Hz) and low-frequency signals (LFS; between 0.3-17 Hz) [3]. Deep-learning models were trained to learn mappings between these ECoG features and phones, speech-sound features, and articulatory gestures, which were then used to output text, synthesize speech audio, and animate a virtual avatar, respectively (FIG. 1).

The system was evaluated using three custom sentence sets containing varying amounts of unique words and sentences named “50-phrase-AAC,” “529-phrase-AAC,” and “1024-word-General.”The first two sets closely mirror corpora preloaded on commercially available augmentative and alternative communication (AAC) devices, designed to let patients express basic concepts and caregiving needs [4]. These two sets were chosen to assess the ability to decode high-utility sentences at a limited and expanded vocabulary level. The 529-phrase-AAC set contained 529 sentences composed of 372 unique words, and 50 high-utility sentences composed of 119 unique words were selected to create the 50-phrase-AAC set. To evaluate how well the system performed with a larger vocabulary containing common English words, the 1024-word-General set was created, containing 9,512 sentences composed of 1,024 unique words sampled from Twitter and movie transcriptions. This set was primarily used to assess how well the decoders could generalize to sentences that the participant did not attempt to say during training with a vocabulary size large enough to facilitate general-purpose communication.

To train the neural-decoding models prior to real-time testing, ECoG data was recorded as the participant silently attempted to speak individual sentences. Learning statistical mappings between the ECoG features and the sequences of phones and speech sound features in the sentences was challenged by the absence of clear timing information in the attempted speech. To overcome the inability to definitively know when the phones and speech units began and ended, a connectionist temporal classification (CTC) loss function was used during training of the neural decoders, which is commonly used in automatic speech recognition to infer sequences of sub-word units (such as phones or letters) from speech waveforms when precise time alignment between the units and the waveforms is unknown [5]. CTC loss was used during training of the text-decoding, speech-synthesis, and articulatory-decoding models to enable prediction of phone probabilities, discrete speech-sound units, and discrete articulator movements, respectively, from the ECoG signals.

FIG. 1 and FIGS. 2A to 2C depict multimodal speech decoding in a participant with vocal-tract paralysis. FIG. 1 illustrates an overview of the speech-decoding pipeline. A brainstem-stroke survivor with anarthria was implanted with a 253-channel high-density electrocorticography (ECoG) array 18 years after injury as part of a clinical trial to decode speech. Neural activity was processed and used to train deep learning models to predict phone probabilities, speech-sound features, and articulatory gestures. These outputs were used to decode text, synthesize audible speech, and animate a virtual avatar, respectively. FIG. 2A provides a sagittal MRI showing brainstem atrophy (in the bilateral pons; red arrow) resulting from stroke. FIG. 2B shows MRI reconstruction of the participant's brain overlaid with the locations of implanted electrodes. The ECoG array was implanted over the participant's lateral cortex, centered on the central sulcus. FIG. 2C shows an example of ECoG features resulting from attempted orofacial movements. The top illustrations depict simple articulatory movements attempted by the participant. The middle depictions illustrate electrode-activation maps demonstrating robust electrode tunings across articulators during attempted movements. Only the electrodes with the strongest responses (top 20%) are shown for each movement type. Color indicates the magnitude of the average evoked high-gamma activity (HGA) response with each type of movement. The bottom plots depict Z-scored trial-averaged evoked HGA responses with each movement type for each of the boxed electrodes in the electrode-activation maps. In each plot, each response trace shows mean±/−standard error across trials and is aligned to the peak activation time.

### 1.3. RESULTS

### 1.3.1. Text Decoding

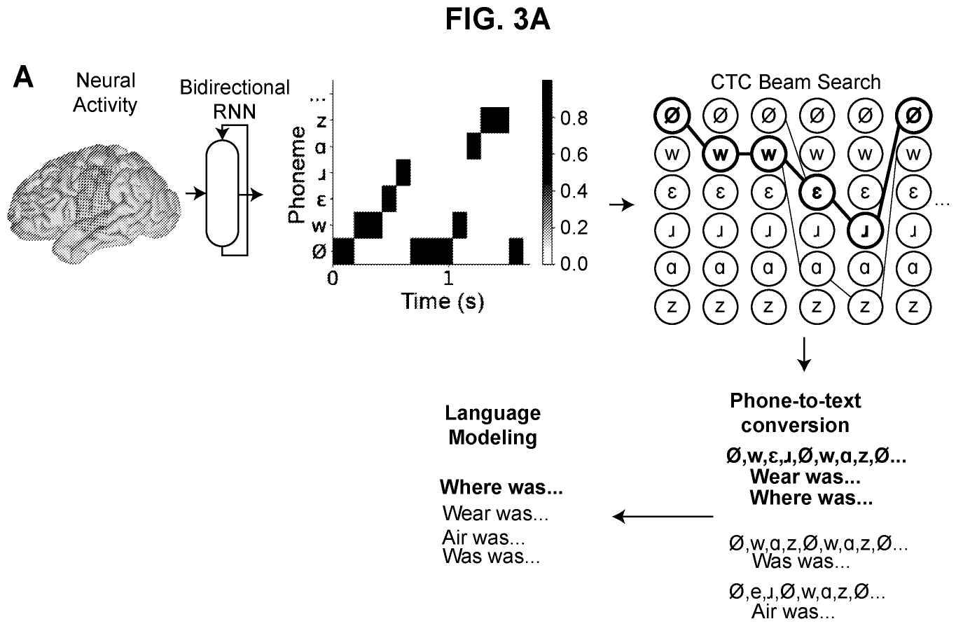

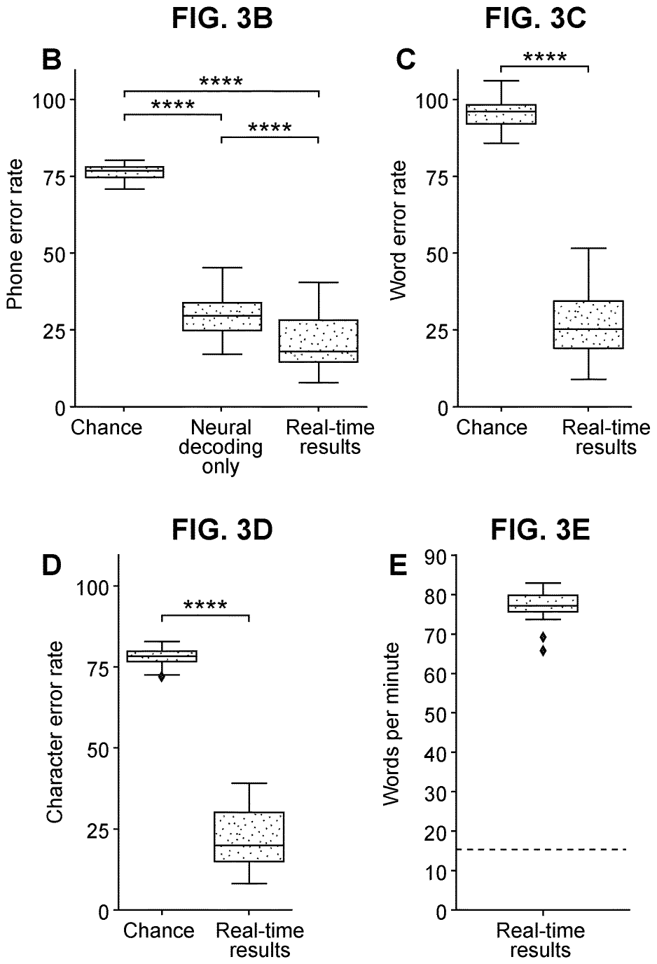

To evaluate real-time performance during the text task condition, text was decoded as the participant attempted to silently say 250 randomly selected sentences from the 1024-word-General set that were not used during model training (FIG. 3A, Video 1). To decode text, features extracted from ECoG signals were streamed starting 500 ms prior to the go cue into a bidirectional recurrent neural network (RNN). Prior to testing, the RNN was trained to predict the probabilities of 39 phones and silence at each time step. A CTC beam search then determined the most likely sentence given these probabilities. First, it created a set of candidate phone sequences that were constrained to form valid words within the 1,024-word vocabulary. Then, it evaluated candidate sentences by combining each candidate's underlying phone probabilities with its linguistic probability using a natural-language model.

To quantify text-decoding performance, standard metrics were used in automatic speech recognition: word error rate (WER), phone error rate (PER), character error rate (CER), and words per minute (WPM). WER, PER, and CER measure the percentage of decoded words, phones, and characters, respectively, that were incorrect.

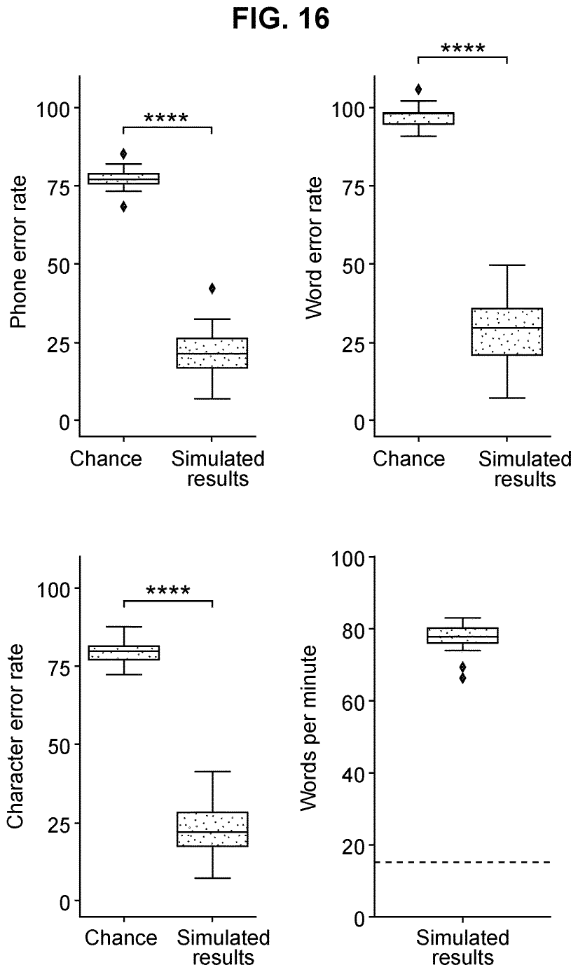

During real-time evaluation 250 randomly selected test sentences were decoded from the corpus that the participant did not attempt to produce for model training, and then error rates were computed across sequential pseudo-blocks of 10-sentence segments. Sentences were decoded with a median PER of 18.5% (99% CI [14.9, 28.5]; FIG. 3B), a median WER of 25.5% (99% CI [19.3, 34.5]; FIG. 3C), and a median CER of 19.9% (99% CI [15.0, 30.1]; FIG. 3C; see Table 1 for example decodes). For all metrics, performance was better than chance, which was computed by re-evaluating performance after using temporally shuffled neural data as the input to the decoding pipeline (P<0.0001 for all three comparisons, two-sided Wilcoxon rank-sum tests with 5-way Holm-Bonferroni correction). The average WER passes the 30% threshold below which speech-recognition applications generally become useful [6] while providing access to a large vocabulary of over 1,000 words, indicating that the approach may be viable in clinical applications. Furthermore, in offline simulations using the same neural data and decoder but with a modified language model and vocabulary containing 42,391 words, an offline WER of 29.8% was achieved (99% CI [21.1, 36.00]; FIG. 16) showing that the approach retains high performance in a large-vocabulary setting.

TABLE 1
Illustrative text decoding examples for the 1024-word-General set. Examples are shown
for various levels of word error rate (WER) during real-time decoding with the 1024-
word-General set. Each percentile value indicates the percent of decoded sentences
that had a WER less than or equal to the WER of the provided example sentence.

| | | WER | Percentile |
|---|---|---|---|
| Target sentence | Decoded sentence | (%) | (%) |
| You should have let me do the talking | You should have let me do the talking | 0 | 46.0 |
| I think I need a little air | I think I need a little air | 0 | 46.0 |
| Do you want to get some coffee | Do you want to get some coffee | 0 | 46.0 |
| What do you get if you finish | Why do you get if you finish | 14 | 46.8 |
| What do you want from us | What do you want for us | 17 | 51.6 |
| You got your wish | You get your wish | 25 | 62.4 |
| No tell me why | So tell me why | 25 | 62.4 |
| You have no right to keep us here | You have no right to be out here | 25 | 62.4 |
| Why would they come to me | Why would they have to be | 33 | 64.8 |
| Why are you looking at me like that | Why are you looking at that | 38 | 66.4 |
| All I told them was the truth | Can I do that was the truth | 43 | 71.6 |
| You got it all in your head | You got here all your right | 43 | 71.6 |
| I would like to watch television | I was that no one television | 67 | 86.0 |
| Do you mind me talking about your stuff | Do you make it out to yourself | 75 | 89.2 |

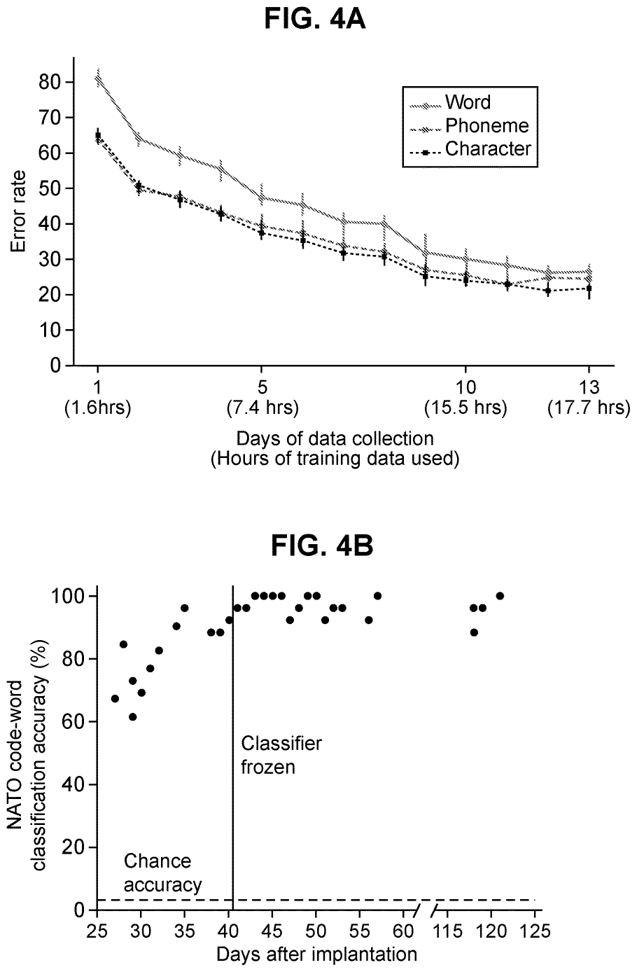

A median real-time decoding rate of 77.6 WPM was observed (99% CI [76.7, 78.4]; FIG. 4A) and a maximum rate of 83.7 WPM. This decoding rate exceeds the participant's typical communication rate using her previous assistive device (14.2 WPM) and is closer to naturalistic speaking rates than has been previously reported with communication neuroprostheses [2] [3] [7-9].

To assess how well the system could decode phones in the absence of a language model and constrained vocabulary, performance was evaluated using just the RNN neural-decoding model (using the most likely phone prediction at each time step instead of the CTC beam search) in an offline analysis. This yielded a median PER of 29.4% (99% CI [26.3, 33.0]; FIG. 3B). This median PER is only 10.9 percentage points higher than the full model, demonstrating that the primary contributor to phone-decoding performance was the neural-decoding RNN model and not the CTC beam search or language model (P<0.0001 for all comparisons to chance and to the full model, two-sided Wilcoxon signed-rank tests with 5-way Holm-Bonferroni correction; Table 2).

TABLE 2
Real-time text-decoding comparisons with
the 1024- word-General sentence set.

| | | Corrected |
|---|---|---|
| Comparison | Statistic¹ | P-value |
| PER Chance vs. Real-time results | 0.00e+00 | 2.98e−07 |
| PER Neural decoding only vs. Real-time results | 0.00e+00 | 2.98e−07 |
| PER Chance vs. Neural decoding only | 0.00e+00 | 2.98e−07 |
| WER Chance vs. Real-time results | 0.00e+00 | 2.98e−07 |
| CER Chance vs. Real-time results | 0.00e+00 | 2.98e−07 |

¹Each comparison is a two-sided Wilcoxon Signed Rank test across n = 25 pseudo-blocks, with 5-way Holm-Bonferroni correction. These comparisons were compated using real-time text-decoding results with the 1024-word-General sentence set, shown in FIG. 2 in the main text.

The relationship between quantity of training data and text-decoding performance was also characterized in offline analyses. For each day of data collection, models were trained on all the data collected on or before that date, then performance was simulated on the real-time blocks. Steadily declining error rates were observed over the course of 13 days of training-data collection (FIG. 4A), during which 9,512 sentence trials were collected at an average rate of about 1.6 hours of training data per day. Overall, these results show that functional speech-decoding performance can be achieved after a relatively short period of data collection compared to prior work [2] [3] and is likely to continue to improve with more data [10].

To assess signal stability, real-time classification performance was measured during a separate NATO-motor task that was collected during each research session with the participant. In each trial of this task, the participant was prompted to attempt to either silently say one of the 26 code words from the NATO phonetic alphabet (alpha, bravo, charlie, and so forth) or perform one of four hand movements (described and analyzed below). A neural-network classifier was trained to predict the most likely NATO code word from a 4-second window of ECoG features (aligned to the task go cue), and real-time performance was evaluated with the classifier during the NATO-motor task (FIG. 4B, Video 2). The model continued to be retrained using available data prior to real-time testing in subsequent sessions until day 40, at which point the classifier was frozen after training it on data from the 1,196 available trials (46 per code word). Across 19 sessions after freezing the classifier, a mean classification accuracy of 96.8% (99% CI [94.5, 98.6]) was observed, with accuracies of 100% obtained on 8 of these sessions. Accuracy remained high after a 61 day pause in recording for the participant to travel. These results illustrate the stability of the cortical-surface neural interface and demonstrate that high performance can be achieved with relatively few training trials and without requiring recalibration.

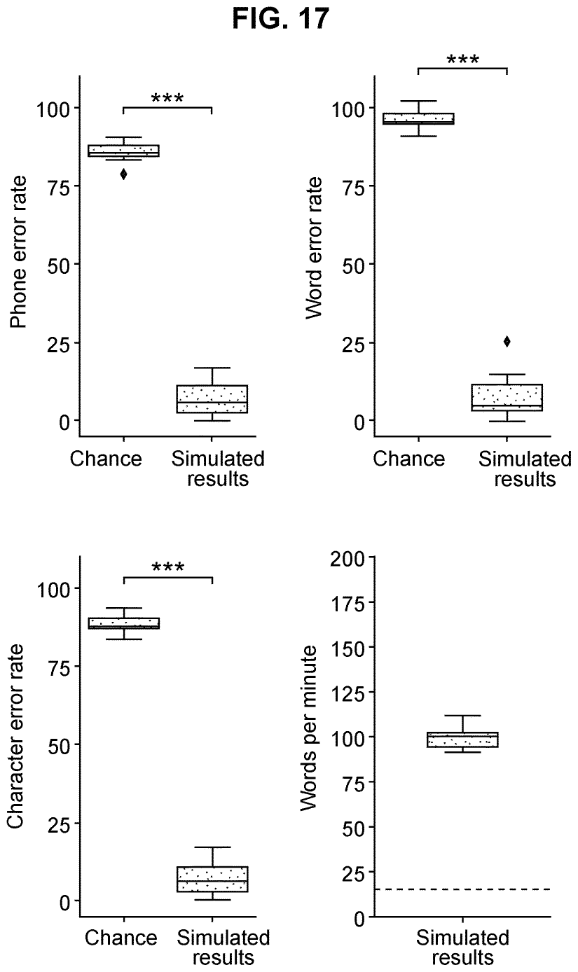

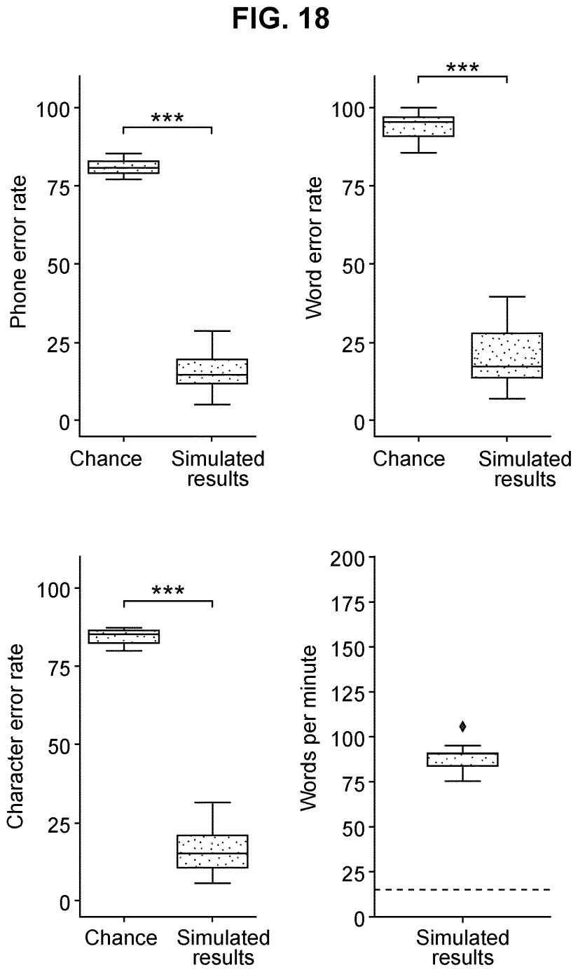

To evaluate model performance on closed sets with repeats across sentences and without pauses in the participant's speech models were trained, then simulated text decoding with the AAC-50 (FIG. 17) and AAC-529 (FIG. 18) sentence sets (Method 1). With the AAC-529 set, a median WER of 17.1% was observed across sentences (99% CI [8.89%, 28.9%]), with a median decoding rate of 89.9 WPM (99% CI [83.6, 93.3]). With the AAC-50 set, a median WER of 4.92% (99% CI [3.18, 14.04]) was observed with median decoding speeds of 101 WPM (99% CI: [95.6, 103]). PERs and CERs for each set are given in FIGS. 17 and 18. The results show the models make more accurate predictions as the set's sizes are reduced, and that the approach of the invention can directly decode faster, continuous speech in the context of closed sentence sets, which are highly valuable for common assistive-communication needs.

FIGS. 3A to 3E and FIGS. 4A to 4B depict high-performance text decoding from neural activity. FIG. 3A is a schematic diagram of the text-decoding algorithm. During attempts to silently speak, a bidirectional recurrent neural network (RNN) decodes neural features into a time series of phone and silence probabilities. Only the first 5 target phones and the silence token (denoted as Ø) are depicted. A CTC beam search processes this time series, searching for the most likely sequence of phones that can be translated into valid words in the vocabulary. An n-gram language model rescores sentences created from these phone sequences to yield the most likely sentence. FIG. 3B depicts median phone error rates with the 1024-word-General sentence set, calculated using shuffled neural data (Chance), neural decoding with the RNN without applying vocabulary constraints or language modeling (Neural decoding only), and the full real-time system (Real-time results) across 25 pseudo-blocks each containing 10 sentence trials. FIG. 3C depicts word error rates for chance and real-time results. FIG. 3D depicts character error rates for chance and real-time results. FIGS. 3B to 3D used a ****P<0.0001, Two-sided Wilcoxon Signed-Rank test with 5-way Holm-Bonferroni correction for multiple comparisons; P-values and statistics are found in Table 2. FIG. 3E depicts communication rates, measured by decoded words per minute. Dashed line denotes previous state-of-the art rate for a speech brain-computer interface (BCI) in a person with paralysis [2]. FIG. 4A depicts offline evaluation of error rates as a function of number of recording days and data quantity. FIG. 4B depicts real-time classification accuracy during attempts to silently say 26 NATO code words across many recording days. The classifier was retrained after every session until it was frozen and no longer updated (vertical line). All box plots in all figures depict mean (horizontal line inside box), 25th and 75th percentiles (box), 25th and 75th percentiles+/−1.5 times the interquartile range (whiskers), and outliers (diamonds).

FIG. 16 provides results of simulated text decoding with a larger vocabulary. Text decoding results were simulated using a 42,391 word vocabulary on the blocks used for real-time evaluation with the 1024-word-General set. Across pseudo-blocks, a median WER of 29.82% (99% CI [21.1, 36.0], median CER of 22.17% (99% CI [17.5, 28.4]), and median PER of 21.4% (99% CI [16.8%, 26.6%]) was achieved. These results demonstrate consistent performance, even with a vocabulary 40× larger than the one used in real-time, demonstrating the natural ability of the phone decoding approach to scale up. The PER, WER, and CER were also significantly better than chance (P<0.0001 for all metrics, Wilcoxon signed-rank test with 3-way Holm-Bonferonni Correction for multiple comparisons, n=25 pseudo-blocks). For PER: stat=0, P=1.79e-8. For CER: stat=0, P=1.79e-8. For WER: stat=0, P=1.79e-8.

FIG. 17 provides results of simulated text decoding on the 50-phrase-AAC sentence set. Text decoding results were simulated on the real-time blocks used for evaluation with the synthesis models. On the 50-phrase-AAC sentence set, extremely high accuracy was achieved. A median PER of 5.63% (99% CI [2.10, 12.0]) was observed. Median WER was 4.92% (99% CI [3.18, 14.0]), and median CER was 5.91% (99% CI [2.21, 11.4]). Speech was decoded at high rates with a median WPM of 101 (99% CI [95.6, 103]). The PER, WER, and CER were also significantly better than chance (P<0.001 for all metrics, Wilcoxon signed-rank test with 3-way Holm-Bonferonni Correction for multiple comparisons). Statistics compare n=15 total pseudo-blocks. For PER: stat=0, P=1.83e-4. For CER: stat=0, P=1.83e-4. For WER: stat=0, P=1.83e-4.

FIG. 18 provides results of simulated text decoding on the 529-phrase-AAC sentence set. Text decoding results were simulated on the real-time blocks used for evaluation of the 529-phrase-AAC sentence set with the synthesis models. A median PER of 17.3 (99% CI [12.6, 20.11) was observed. Median WER was 17.1% (99% CI [8.89, 28.9]), and median CER was 15.2% (99% CI [10.1, 22.7]). Speech was decoded at high rates with a median WPM of 89.9 (99% CI [83.6, 93.3]). The PER, WER, and CER were also significantly better than chance (P<0.001 for all metrics, two-sided Wilcoxon signed-rank test with 3-way Holm-Bonferonni Correction for multiple comparisons). Statistics compare n=15 total pseudo-blocks. For PER: stat=0, P=1.83e-4. For CER: stat=0, P=1.83e-4. For WER: stat=0, P=1.83e-4.

### 1.3.2. Speech Synthesis

An alternative approach to text decoding is to synthesize speech sounds directly from recorded neural activity, which could offer a pathway towards more naturalistic and expressive communication for someone who is unable to speak. It has not previously been shown that intelligible speech can be synthesized from neural activity in someone who is paralyzed.

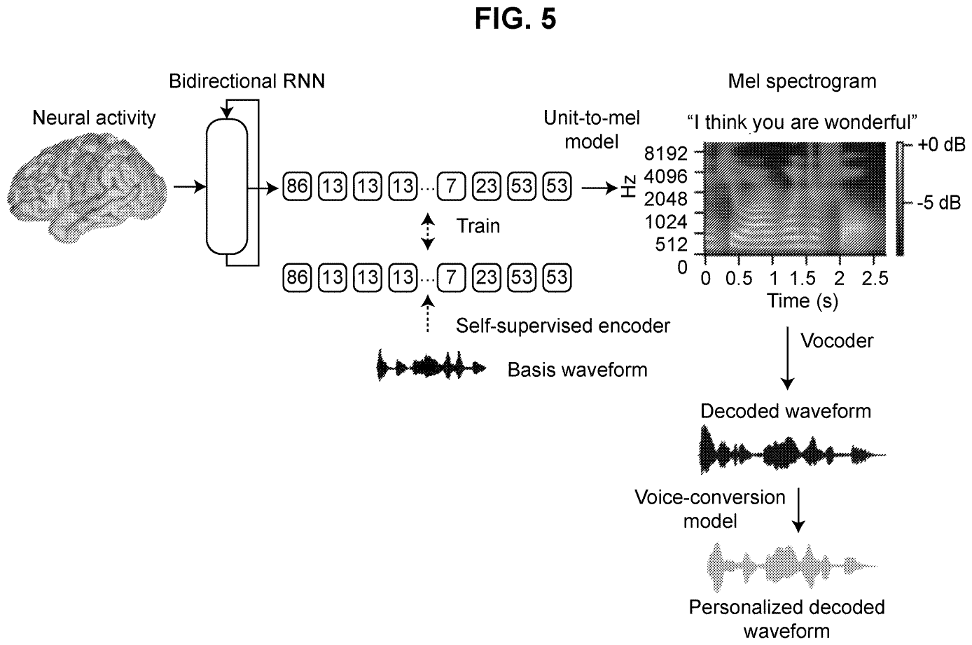

To assess this, real-time speech synthesis was performed by transforming the participant's neural activity directly into audible speech as she attempted to silently speak during the audio-visual task condition (FIG. 5, Videos 3 and 4). To synthesize speech, time windows of neural activity were passed around the go cue into a bidirectional RNN. Prior to testing, the RNN was trained to predict the probabilities of 100 discrete speech units at each time step. To create the reference speech-unit sequences for training, HuBERT, a self-supervised speech-representation learning model, was used [13]. HuBERT was chosen for its ability to encode a continuous speech waveform into a temporal sequence of discrete speech units that captures latent phonetic and articulatory representations [14]. Because the participant cannot speak, reference speech waveforms were acquired from a recruited speaker for the AAC sentence sets or via a text-to-speech algorithm for the 1024-word-General set. A CTC loss function was used during training to enable the RNN to learn mappings between the ECoG features and speech units derived from these reference waveforms without having precise alignment between the participant's silent speech attempts and the reference waveforms. Note the participant never heard the basis or reference waveforms and was not instructed to alter the style or pacing of her attempts to match the basis or reference waveforms in any way. After predicting the unit probabilities, the most likely unit at each time step was passed into a pre-trained unit-to-speech model that first generated a mel spectrogram and vocoded this mel spectrogram into an audible speech waveform in real time [15] [16]. Offline, a voice-conversion model trained on a brief segment of the participant's speech (recorded before her injury) was used to process the decoded speech into the participant's own personalized synthetic voice (Video 5).

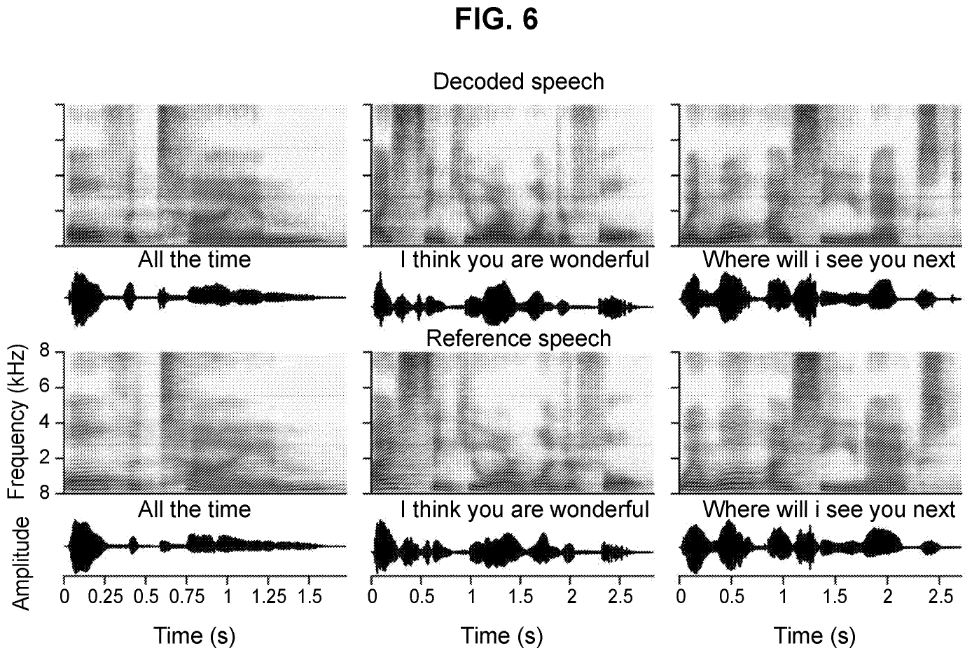

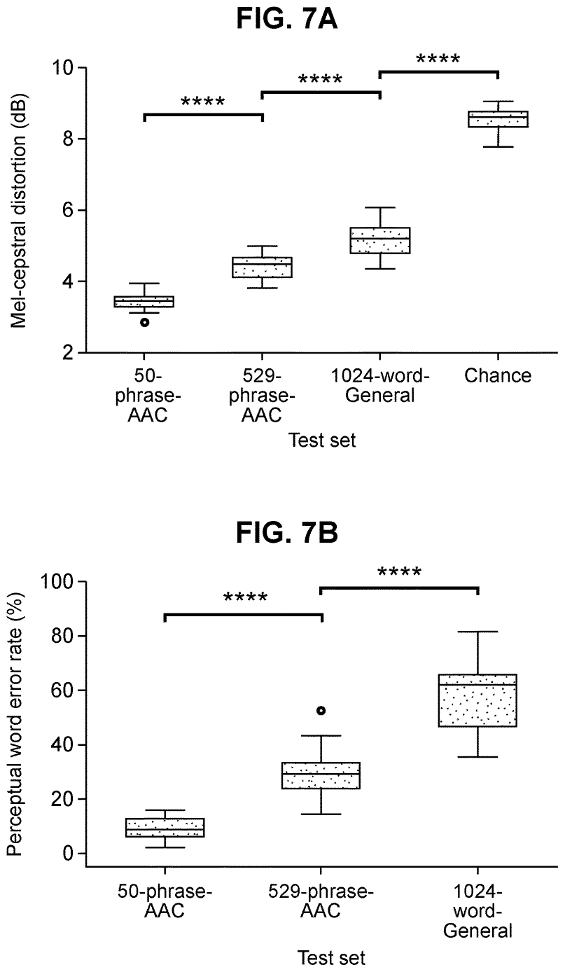

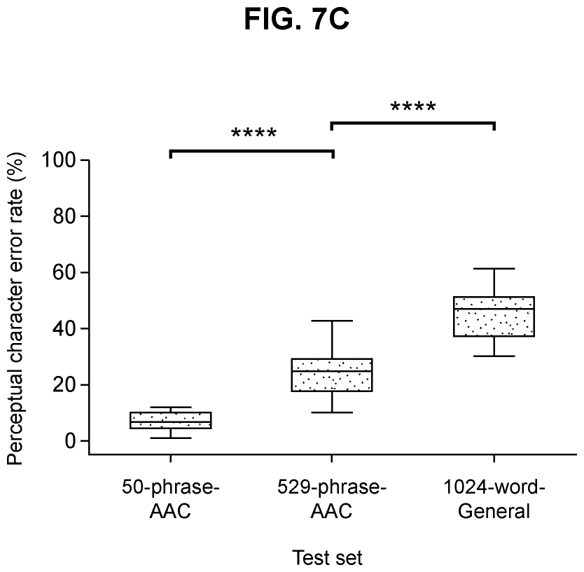

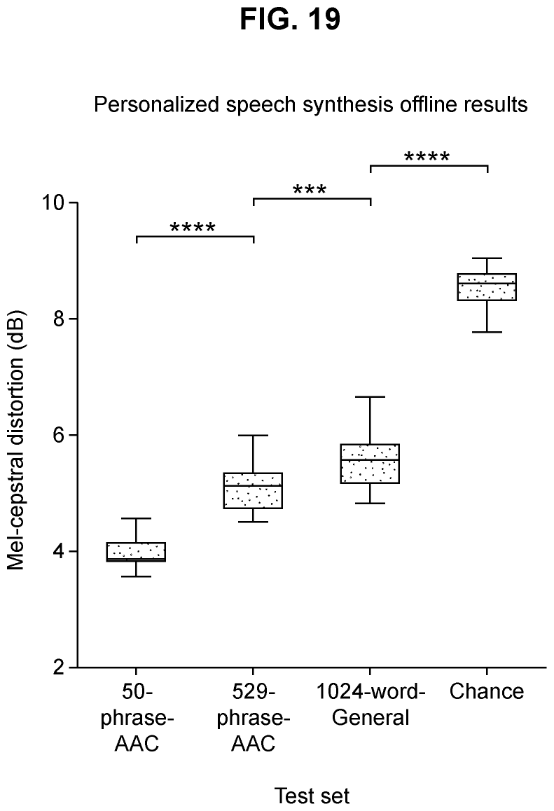

From speech-synthesis outputs during real-time testing, it was qualitatively observed that decoded spectrograms shared both fine-grained and broad time-scale information with corresponding reference spectrograms (FIG. 6). To quantitatively assess the quality of the decoded speech, the mel-cepstral distortion (MCD) metric was used, which measures the similarity between two sets of mel-cepstral coefficients (which are speech-relevant acoustic features) and is commonly used to evaluate speech-synthesis performance [17] [18]. Lower MCD indicates stronger similarity. Mean MCDs of 3.45 (99% CI [3.25, 3.82]), 4.49 (99% CI [4.07, 4.67]), and 5.21 (99% CI [4.74, 5.51]) dB were achieved for the 50-phrase-AAC, 529-phrase-AAC, and 1024-word-General sets, respectively (FIG. 7A). Similar MCD performance was observed on the participant's personalized voice (FIG. 19; Table 3). Performance increased as the number of unique words and sentences in the sentence set decreased but was always better than chance (all P<0.0001, two-sided Wilcoxon rank-sum tests with 19-way Holm-Bonferroni correction; chance MCDs were measured using waveforms generated by passing temporally shuffled ECoG features through the synthesis pipeline). Further, these MCDs are comparable to those observed with text-to-speech synthesizers [18] and better than what has been reported in prior neural-decoding work with participants that were able to speak naturally [12].

TABLE 3
Participant's personalized voice MCD comparisons.

| | | | Corrected |
|---|---|---|---|
| Dataset 1 | Dataset 2 | Statistic | P-value |
| 1024-word-General B3 MCD | 50-phrase-AAC chance MCD | 2.07e+03 | 1.62e−42 |
| 1024-word-General B3 MCD | 1024-word-General chance MCD | 1.88e+02 | 1.42e−32 |
| 50-phrase-AAC B3 MCD | 1024-word-General B3 MCD | 4.98e+03 | 5.37e−26 |
| 50-phrase-AAC B3 MCD | 50-phrase AAC chance MCD | 1.00e+00 | 9.39e−26 |
| 529-phrase-AAC B3 MCD | 529-phrase-AAC chance MCD | 7.80e+01 | 3.28e−25 |
| 50-phrase-AAC B3 MCD | 529-phrase-AAC B3 MCD | 6.04e+03 | 8.00e−12 |
| 529-phrase-AAC B3 MCD | 1024-word-General B3 MCD | 1.16e+04 | 2.34e−04 |

¹ Across dataset comparisons are two-sided Mann-Whitney U-tests whereas within-dataset comparisons are two-sided Wilcoxon signed-rank paired tests; all with 7-way Holm-Bonferonni correction for multiple comparisons. Comparisons use n = 20 pseudo-blocks for the 1024-word-General sentence set. and n = 15 pseudo-blocks for the AAC setnence sets.

Human-transcription assessments are a standard method to quantify the perceptual accuracy of synthesized speech [19]. To directly assess the intelligibility of the synthesized speech waveforms, perceptual free-transcription accuracy was measured from evaluators on a crowd-sourcing platform (Amazon Mechanical Turk). In this assessment, evaluators listened to the synthesized speech waveforms and then transcribed what they heard into text. Perceptual word and character error rates (WERs and CERs, respectively) were then computed by comparing these transcriptions to the ground-truth sentence texts. For each test trial, 11 evaluators transcribed the same synthesized speech waveform, and then the median error rate was used across evaluators to obtain a single WER and CER value for that trial. Median perceptual WERs of 8.33% (99% CI [5.17, 14.0]), 29.2% (99% CI [22.5, 33.3]), 61.9% (99% CI [46.4, 65.4]) and median perceptual CERs of 6.30% (99% CI [3.69, 10.1]), 24.6% (99% CI [16.5, 29.3]), and 46.7% (99% CI [36.6, 51.7]) were achieved across test trials for the 50-phrase-AAC, 529-phrase-AAC, and 1024-word-General sets, respectively (FIGS. 7B and 7C). Similar to the MCD results, perceptual WERs and CERs improved as the number of unique words and sentences in the sentence set decreased (all P<0.0001, 2-sided Wilcoxon rank-sum tests with 19-way Holm-Bonferroni correction; chance perceptual WERs and CERs were measured by shuffling the mapping between the transcriptions and the ground-truth sentence texts). For the AAC sentence sets, evaluators often transcribed the decoded speech with perfect or near-perfect accuracy (119/150 trials and 79/150 trials had a perceptual WER of less than 15% for the 50-phrase-AAC and 500-phrase-AAC sets respectively). Together, these results demonstrate it is possible to synthesize intelligible speech from brain activity in a person with paralysis.

TABLE 4
Real-time audio-visual synthesis comparisons.

| | | | Corrected |
|---|---|---|---|
| Dataset 1 | Dataset 2 | Statistic | P-value |
| 1024-word-General MCD | 50-phrase-AAC chance MCD | 1.10e+03 | 1.68e−48 |
| 50-phrase-AAC WER | 1024-word-General WER | 3.94e+03 | 3.09e−33 |
| 50-phrase-AAC CER | 1024-word-General CER | 3.96e+03 | 4.56e−33 |
| 1024-word-General MCD | 1024-werd-General chance MCD | 7.70e+01 | 7.30e−33 |
| 1024-word-General CER | 1024-word-General chance CER | 1.96e+02 | 4.01e−32 |
| 1024-word-General WER | 1024-word-Operat chance WER | 2.55e+01 | 1.10e−29 |
| 50-phrase-AAC MCD | 1024-word-General MCD | 4.53e+03 | 6.59e−28 |
| 50-phrase-AAC WER | 50-phrase AAC chance WER | 0.00e+00 | 1.35e−25 |
| 50-phrase-AAC MCD | 50-phrase-AAC chance MCD | 1.00e+00 | 2.58e−25 |
| 50-phrase-AAC CER | 50-phrase-AAC chance CER | 3.00e+00 | 2.58e−25 |
| 529-phrase-AAC MCD | 529-phrase-AAC chance MOD | 2.70e+01 | 3.56e−25 |
| 529-phrase-AAC CER | 529 phrase-AAC chance CER | 4.10e+01 | 6.13e−25 |
| 529-phrase-AAC WER | 529-phrase-AAC chauce WER | 6.20e+01 | 7.52e−24 |
| 529-phrase-AAC WER | 1024-word-General WER | 8.34e+03 | 3.12e−12 |
| 529-phrase-AAC CER | 1024-word-General CER | 9.06e+03 | 6.45e−10 |
| 50-phrase-AAC MCD | 529-phrase-AAC MCD | 7.12e+03 | 1.56e−07 |
| 50-phrase-AAC CER | 529-phrase-AAC CER | 7.79e+03 | 2.43e−07 |
| 50-phrase-AAC WER | 529-phrase AAC WER | 7.83e+03 | 2.43e−07 |
| 529-phrase-AAC MCD | 1024-word-General MCD | 1.04e+04 | 7.08e−07 |

¹ Across dataset comparisons use two-sided Mann-Whitney [symbol] whereas within-dataset comparisons are two-sided Wilcoxon [symbol] . All [symbol] Holm-Bonferroni correction. We [symbol] the AAC [symbol] and n = 20 [symbol] for the 1024-word-General sentence set. [symbol]
indicates data missing or illegible when filed

FIG. 5, FIG. 6, and FIGS. 7A to 7C depict intelligible speech synthesis from neural activity. FIG. 5 is a schematic diagram of the speech-synthesis decoding algorithm. During attempts to silently speak, a bidirectional recurrent neural network (RNN) decodes neural features into a time series of discrete speech units. The RNN was trained using reference speech units computed by applying a large pretrained acoustic model (HuBERT) on basis waveforms. Predicted speech units are then transformed into the mel spectrogram and vocoded into audible speech. The decoded waveform is played back to the participant in real time after a brief delay. Offline, the decoded speech was transformed to be in the participant's personalized synthetic voice using a voice-conversion model. FIG. 5 top: three example decoded spectrograms and waveforms (top) from the 529-phrase-AAC sentence set. Bottom: the corresponding reference spectrograms and waveforms representing the decoding targets. FIG. 7A depicts mel-cepstral distortions (MCDs) for the decoded waveforms. Lower MCD indicates better performance. Chance waveforms were computed by shuffling electrode indices in the test data for the 50-phrase-AAC set with the same synthesis pipeline. FIG. 7B shows perceptual word error rates from untrained human evaluators via a transcription task. FIG. 7C shows perceptual character error rates from the same human-evaluation results as FIG. 7B. In FIGS. 7A to 7C, ****P<0.0001, Mann-Whitney U-test with 19-way Holm-Bonferroni correction for multiple comparisons; all non-adjacent comparisons were also significant (P<0.0001; not depicted); n=15 pseudo-blocks for the AAC sets, n=20 pseudo-blocks for 1024-word-General set. P-values and statistics in Table 4. In FIG. 6 and FIGS. 7A to 7C, all decoded waveforms, spectrograms, and quantitative results use the non-personalized voice (see FIG. 19 and Table 3 for results with the personalized voice).

FIG. 19 provides Mel-cepstral distortions (MCDs) using a personalized voice tailored to the participant. The Mel-cepstral distortion (MCDs) was calculated between decoded speech with the participant's personalized voice and reference waveforms for the 529-phrase-AAC, 50-phrase-AAC, and 1024-word-General set. Lower MCD indicates better performance. Mean MCDs of 3.87 (99% CI [3.83, 4.45]), 5.12 (99% CI [4.41, 5.35]), and 5.57 (99% CI [5.17, 5.90]) dB were achieved for the 50-phrase-AAC (N=15 pseudo-blocks), 529-phrase-AAC (N=15 pseudo-blocks), and 1024-word-General sets (N=20 pseudo-blocks). Chance MCDs were computed by shuffling electrode indices in the test data with the same synthesis pipeline. The MCDs of all sets are significantly lower than the chance. 529-phrase-AAC vs. 1024-word-General * * *=P≤0.001, otherwise all * * **=P≤0.0001. Two-sided Wilcoxon rank-sum tests for comparisons within-dataset and Mann-Whitney U-test outside of dataset with 9-way Holm-Bonferroni correct.

### 1.3.3. Animating Articulation for a Personalized BCI Avatar

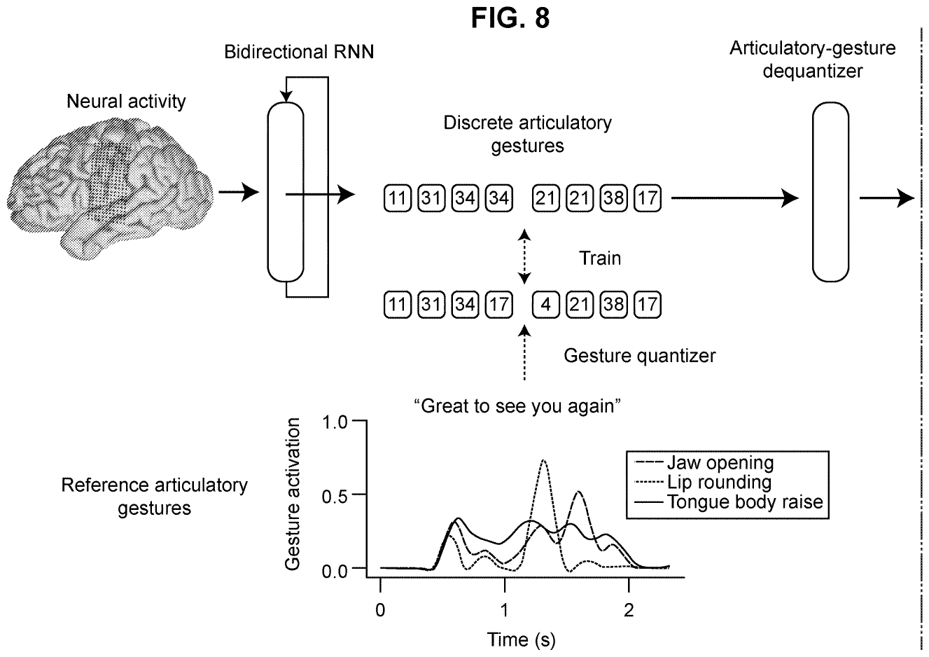

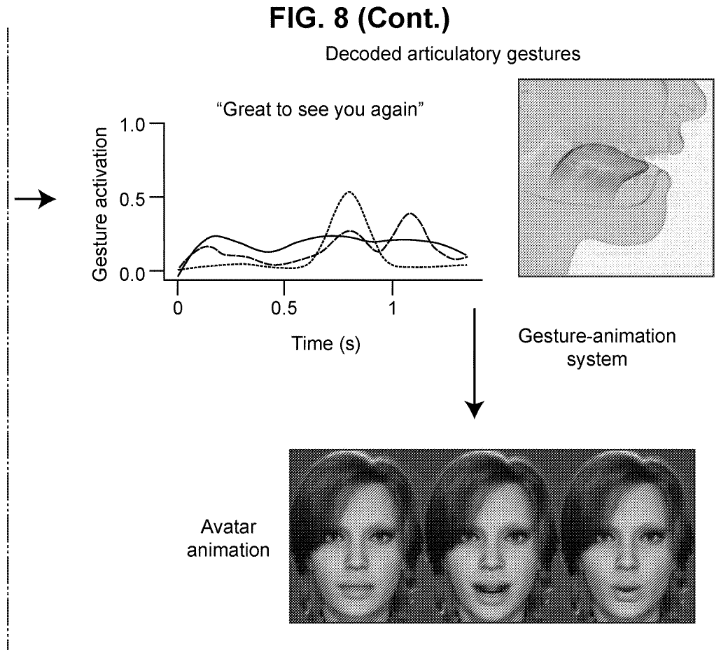

Face-to-face audio-visual communication offers multiple advantages over solely audio-based communication. Previous studies show that non-verbal facial gestures often account for a significant portion of the perceived fecling and attitude of a speaker [20] [21] and that face-to-face communication enhances social connectivity [22] and intelligibility [23]. Therefore, animation of a facial avatar to accompany synthesized speech and further embody the user is a promising means toward naturalistic communication. To this end, a facial-avatar BCI was developed to decode neural activity into articulatory speech gestures and render a dynamically moving virtual face during the audio-visual task condition (FIG. 8).

To synthesize the avatar's motion an avatar-animation system was used that was designed to transform speech signals into accompanying facial-movement animations for applications in games, TV, film and communication (Speech Graphics Ltd, Edinburgh, Scotland). This technology uses speech-to-gesture methods that predict articulatory gestures (Table 5) from sound waveforms then synthesizes the avatar animation from these gestures [28]. A 3-D virtual environment was designed to display the avatar to the participant during testing. Before testing, the participant selected an avatar from multiple potential candidates.

TABLE 5
Articulatory gesture descriptions.

| Articulatory gesture | Description |
|---|---|
| Tongue tip raise | Raise the tip of the tongue |
| Tongue retraction | Retraction of the tongue |
| Tongue body raise | Raise the body portion of tongue |
| Tongue advance | Tongue extends out of the mouth |
| Rounding | Lips are puckered |
| Pinching | Mouth corners are pressed and pulled back |
| Nostril flare | Nostrils open (e.g during a breath) |
| Upper lip pull | Upper lip is pulled upward |
| Lower lip tuck | Lower lip is tucked over teeth, as in during an F |
| Lower lip push | Lower lip pushed upward by movement in chin |
| Lower lip pull | Lower lip pulled down and gums exposed |
| Lip flare | Lips move to make ‘SH’ sound |
| Jaw opening | Opening of the jaw |
| Compression | Lips pursed without protrusion |
| Adduction | Upper lips rolls down and in, as in during an |
| | ‘M’ sound |

¹ Reference articulatory gestures sampled at 100 Hz and generate vis Speech Graphics’ SG Com acoustic-to-articulatory inversion model.
² The gestures are continuous-valued activations of muscle movements that correspond to the degree each gesture is activated.

Two approaches were implemented for animating the avatar: a direct approach and an acoustic approach. The direct approach was used for offline analyses to evaluate if articulatory movements could be directly inferred from neural activity without the use of a speech-based intermediate, which has implications for potential future uses of an avatar that are not based on speech representations, including non-verbal facial expressions. The acoustic approach was used for real-time audio-visual synthesis because it provided low-latency synchronization between decoded speech audio and avatar movements.

For the direct approach, a bidirectional RNN was trained with CTC loss to learn a mapping between ECG features and reference discretized articulatory gestures. These articulatory gestures were obtained by passing the reference waveforms through the animation system's speech-to-gesture model. The articulatory gestures were then discretized using a vector quantized variational autoencoder (VQ-VAE) [29]. During testing, the RNN was used to decode the discretized articulatory gestures from neural activity and then de-quantized them into continuous articulatory gestures using the VQ-VAE's decoder. Finally, the gesture-to-animation subsystem was used to animate the avatar face from the continuous gestures.

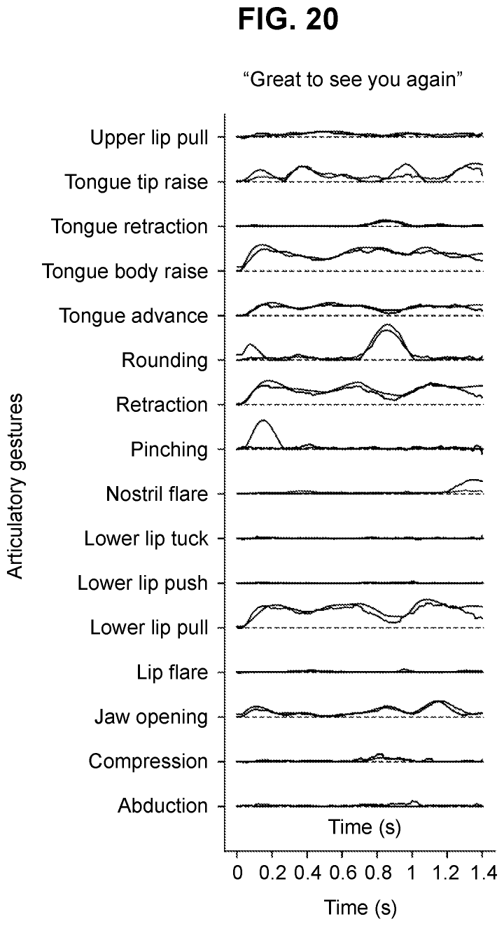

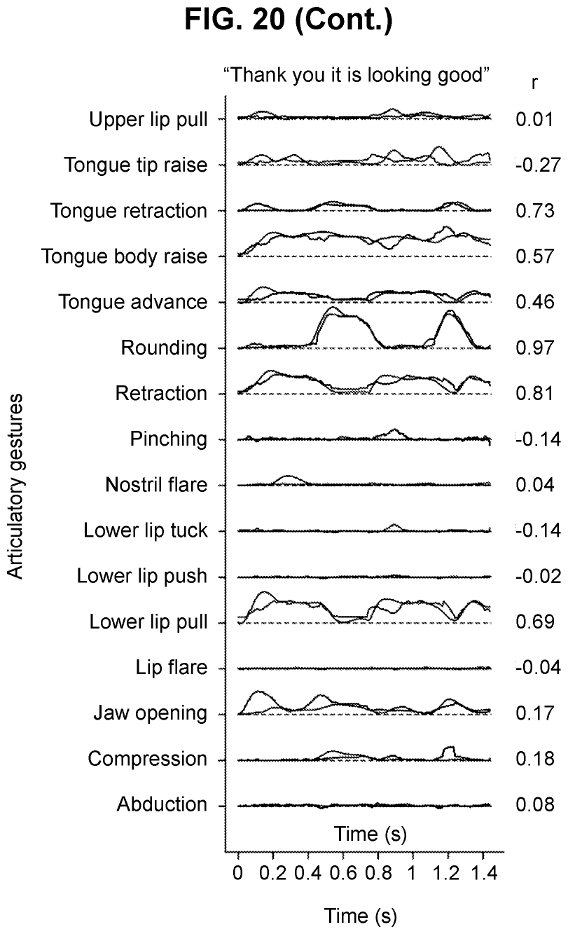

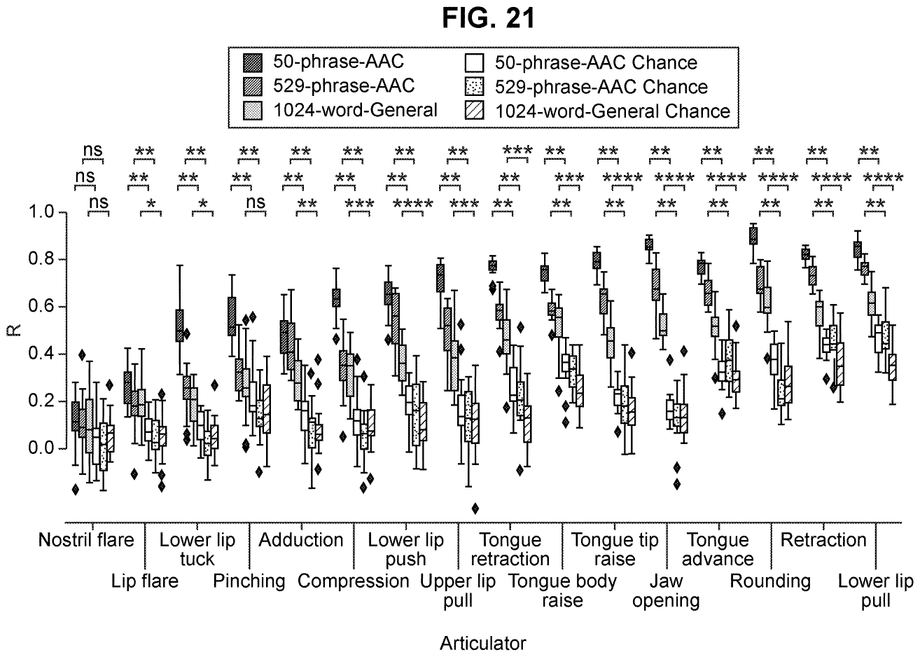

It was found that the direct approach produced articulatory gestures that were strongly correlated with reference articulatory gestures across all datasets (FIG. 20 and FIG. 21; Table 6), highlighting the system's ability to decode articulatory information from brain activity.

TABLE 6
Comparisons for articulatory gesture decoding using the direct-decoding approach.

| | | | | Corrected |
|---|---|---|---|---|
| Articulator | Dataset 1 | Dataset 2 | Statistic¹ | P-value² |
| Upper lip pull | 1024-word-General | 1024-word-General Chance | 4.00e+00 | 8.01e−05 |
| Upper lip pull | 50-phrase AAC | 50-phrase-AAC Chance | 0.00e+00 | 9.77e−04 |
| Upper lip pull | 529-phrase-AAC | 529-phrase-AAC Chance | 0.00e+00 | 9.77e−04 |
| Lower lip pull | 1024-word-General | 1024-word-General Chance | 0.00e+00 | 3.05e−05 |
| Lower lip pull | 50-phrase AAC | 50-phrase-AAC Chance | 8.00e+00 | 9.77e−04 |
| Lower lip pull | 529-phrase-AAC | 529-phrase-AAC Chance | 0.00e+00 | 9.77e−04 |
| Nostril flare | 1024-word-General | 1024-word-General Chance | 7.80e+01 | 3.30e−01 |
| Nostril flare | 50-phrase AAC | 50-phrase-AAC Chance | 1.80e+01 | 1.51e−02 |
| Nostril flare | 529-phrase-AAC | 529-phrase-AAC Chance | 2.60e+01 | 5.54e−02 |
| Lower lip tuck | 1024-word-General | 1024-word-General Chance | 2.10e+01 | 3.40e−03 |
| Lower lip tuck | 50-phrase AAC | 50-phrase-AAC Chance | 0.00e+00 | 9.77e−04 |
| Lower lip tuck | 529-phrase-AAC | 529-phrase-AAC Chance | 2.00e+00 | 9.77e−04 |
| Jaw opening | 1024-word-General | 1024-word-General Chance | 0.00e+00 | 3.05e−05 |
| Jaw opening | 50-phrase AAC | 50-phrase-AAC Chance | 0.00e+00 | 9.77e−04 |
| Jaw opening | 529-phrase-AAC | 529-phrase-AAC Chance | 0.00e+00 | 9.77e−04 |
| Compression | 1024-word-General | 1024-word-General Chance | 3.00e+00 | 7.63e−05 |
| Compression | 50-phrase AAC | 50-phrase-AAC Chance | 0.00e+00 | 9.77e−04 |
| Compression | 529-phrase-AAC | 529-phrase-AAC Chance | 0.00e+00 | 9.77e−04 |
| Adduction | 1024-word-General | 1024-word-General Chance | 9.00e+00 | 3.15e−04 |
| Adduction | 50-phrase AAC | 50-phrase-AAC Chance | 0.00e+00 | 9.77e−04 |
| Adduction | 529-phrase-AAC | 529-phrase-AAC Chance | 0.00e+00 | 9.77e−04 |
| Lip flare | 1024-word-General | 1024-word-General Chance | 2.20e+01 | 3.40e−03 |
| Lip flare | 50-phrase AAC | 50-phrase-AAC Chance | 0.00e+00 | 9.77e−04 |
| Lip flare | 529-phrase-AAC | 529-phrase-AAC Chance | 4.00e+00 | 9.77e−04 |
| Tongue tip raise | 1024-word-General | 1024-word-General Chance | 0.00e+00 | 3.05e−05 |
| Tongue tip raise | 50-phrase AAC | 50-phrase-AAC Chance | 0.00e+00 | 9.77e−04 |
| Tongue tip raise | 529-phrase-AAC | 529-phrase-AAC Chance | 0.00e+00 | 9.77e−04 |
| Tongue body raise | 1024-word-General | 1024-word-General Chance | 3.00e+00 | 7.63e−05 |
| Tongne body raise | 50-phrase AAC | 50-phrase-AAC Chance | 0.00e+00 | 9.77e−04 |
| Tongue body raise | 529-phrase-AAC | 529-phrase-AAC Chance | 0.00e+00 | 9.77e−04 |
| Rounding | 1024-word-General | 1024-word-General Chance | 0.00e+00 | 3.05e−05 |
| Rounding | 50-phrase AAC | 50-phrase-AAC Chance | 0.00e+00 | 9.77e−04 |
| Rounding | 529-phrase-AAC | 529-phrase-AAC Chance | 0.00e+00 | 9.77e−04 |
| Lower lip push | 1024-word-General | 1024-word-General Chance | 0.00e+00 | 3.05e−05 |
| Lower lip push | 50-phrase AAC | 50-phrase-AAC Chance | 0.00e+00 | 9.77e−04 |
| Lower lip push | 529-phrase-AAC | 529-phrase-AAC Chance | 0.00e+00 | 9.77e−04 |
| Retraction | 1024-word-General | 1024-word-General Chance | 0.00e+00 | 3.05e−05 |
| Retraction | 50-phrase AAC | 50-phrase-AAC Chance | 0.00e+00 | 9.77e−04 |
| Retraction | 529-phrase-AAC | 529-phrase-AAC Chance | 0.00e+00 | 9.77e−04 |
| Tongue retraction | 1024-word-General | 1024-word-General Chance | 1.00e+00 | 3.43e−05 |
| Tongue retraction | 50-phrase AAC | 50-phrase-AAC Chance | 0.00e+00 | 9.77e−04 |
| Tongue retraction | 529-phrase-AAC | 529-phrase-AAC Chance | 0.00e+00 | 9.77e−04 |
| Pinching | 1024-word-General | 1024-word-General Chance | 5.10e+01 | 8.81e−02 |
| Pinching | 50-phrase AAC | 50-phrase-AAC Chance | 0.00e+00 | 9.77e−04 |
| Pinching | 529-phrase-AAC | 529-phrase-AAC Chance | 1.00e+00 | 9.77e−04 |
| Tongue advance | 1024-word-General | 1024-word-General Chance | 0.00e+00 | 3.05e−05 |
| Tongue advance | 50-phrase AAC | 50-phrase-AAC Chance | 0.00e+00 | 9.77e−04 |
| Tongue advance | 529-phrase-AAC | 529-phrase-AAC Chance | 0.00e+00 | 9.77e−04 |

¹Each comparison is a two-sided Wilcoxon signed-rank test between correlations from real-time decoded articulatory gestures (generated using the direct approach) and reference gestures across n = 20 pseudo-blocks for the 1024-word-General sentence set and n = 15 pseudo-blocks for the 50-phrase-AAC and 529-phrase-AAC sentence sets.
²16-way Holm-Bonferroni correction for multiple comparisons (corresponding to the 16 gesture categories).

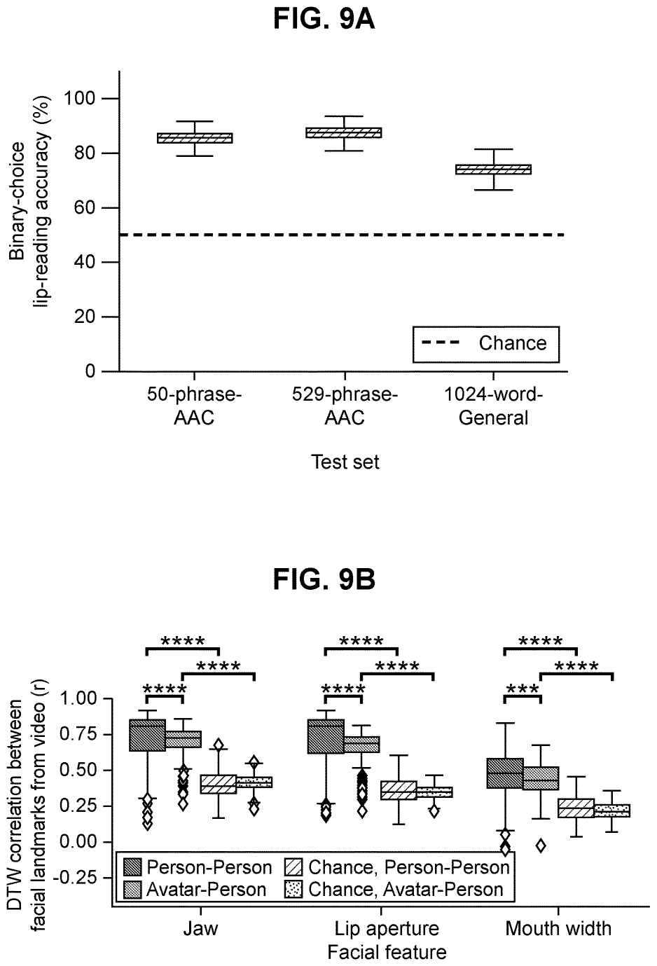

Direct-decoding results were then evaluated by measuring the perceptual accuracy of the avatar. Evaluators on a crowd-sourcing platform (Amazon Mechanical Turk) watched silent videos of the decoded avatar animations and, for each video, were asked to identify to which of two sentences the video corresponded. One sentence was the ground-truth sentence while the other was randomly selected from the set of test sentences. The median bootstrapped accuracy was used across six evaluators to represent the final accuracy for each sentence. Median accuracies of 85.7% (99% CI [79.0, 92.0]), 87.7% (99% CI [79.7, 93.7]), and 74.3% (99% CI [66.7, 80.8]) were obtained across the 50-phrase-AAC, 529-phrase-AAC, and 1024-word-General sets, demonstrating that the avatar renderings conveyed perceptually meaningful speech-related facial movements (FIG. 9B).

Next, the facial-avatar movements generated during direct decoding were compared with real movements made by healthy speakers. Videos of eight healthy volunteers were recorded as they read aloud sentences from the 1024-word-General set. A facial-keypoint recognition model was then applied (dlib) [30] to avatar and healthy-speaker videos to extract trajectories important for speech; jaw opening, lip aperture, and mouth width. For each pseudo-block of 10 test sentences and each trajectory, the mean correlations were computed across sentences between the trajectory values for each possible pair of corresponding videos (36 total combinations with one avatar and eight healthy-speaker videos). Prior to calculating correlations between two trajectories for the same sentence, dynamic time warping (DTW) was applied to account for variability in timing. It was found that the jaw opening, lip aperture, and mouth width of the avatar and healthy speakers were well correlated with median values of 0.733 (99% CI [0.711, 0.748]), 0.690 (99% CI [0.663, 0.714]), and 0.446 (99% CI [0.417, 0.470]) respectively (FIG. 9B). Although correlations amongst pairs of healthy speakers were significantly higher than between the avatar and healthy speakers (all P<0.0001, two-sided Mann-Whitney U-test with 9-way Holm-Bonferroni correction; Table 7) there was a large degree of overlap between the two distributions, illustrating that the avatar reasonably approximated the expected articulatory trajectories relative to natural variances between healthy speakers. Correlations for both distributions were significantly above chance, which was calculated by temporally shuffling the human trajectories and then recomputing correlations with DTW (all P<0.0001, two-sided Mann-Whitney U-test with 9-way Holm-Bonferroni correction; Table 7).

TABLE 7
Comparisons for dlib traces with the direct approach.

| | | | | Corrected |
|---|---|---|---|---|
| Articulator | Dataset 1 | Dataset 2 | Statistic¹ | P-value² |
| Jaw | Avatar-Person | Person-Person | 2.49e+04 | 1.00e−12 |
| Jaw | Avatar-Person | Chance, Avatar-Person | 2.20e+04 | 1.20e−41 |
| Jaw | Person-Person | Chance, Person-Person | 2.56e+05 | 2.58e−114 |
| Lip aperture | Avatar-Person | Person-Person | 2.23e+04 | 1.11e−16 |
| Lip aperture | Avatar-Person | Chance, Avatar-Person | 2.19e+04 | 3.37e−41 |
| Lip aperture | Person-Person | Chance, Person-Person | 2.57e+05 | 9.39e−117 |
| Mouth width | Avatar-Person | Person-Person | 3.32e+04 | 7.36e−04 |
| Mouth width | Avatar-Person | Chance, Avatar-Person | 2.18e+04 | 2.74e−40 |
| Mouth width | Person-Person | Chance, Person-Person | 2.57e+05 | 8.35e−117 |

¹Each comparison is a two-sided Mann-Whitney U-test between dlib facial- [symbol] feature correlations for the avatar (decoded using the direct approach) and healthy-speaker videos, with n = 19 pseudo-blocks for each of the 8 speakers (yielding 152 data points for avatar-person comparisons) and n = 19 pseudo-blocks for each of the 28 combinations of pairs of speakers (yielding 532 data points for person-person comparisons).
²9-way Holm-Bonferroni correction for multiple comparisons. [symbol]
indicates data missing or illegible when filed

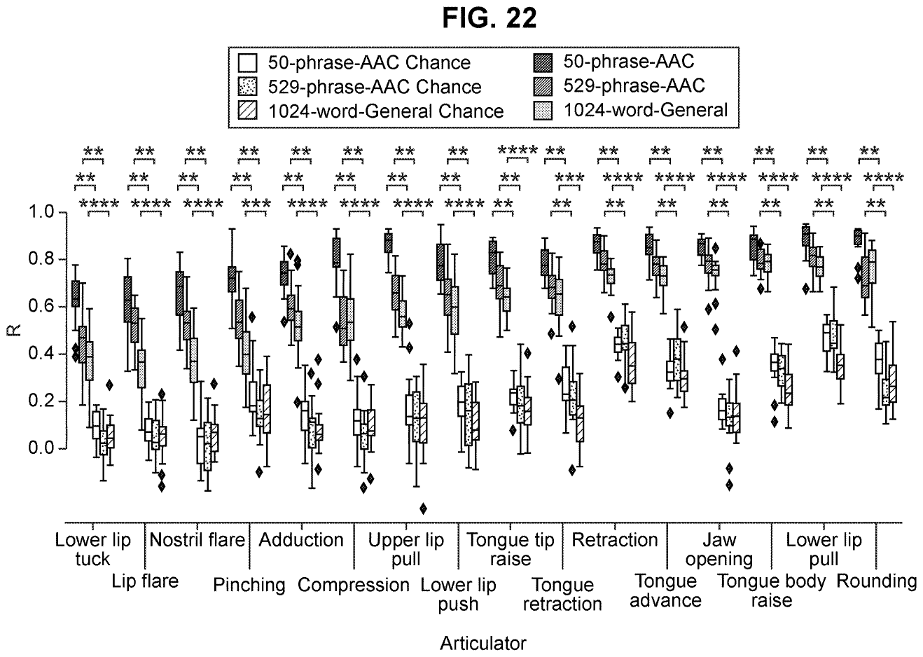

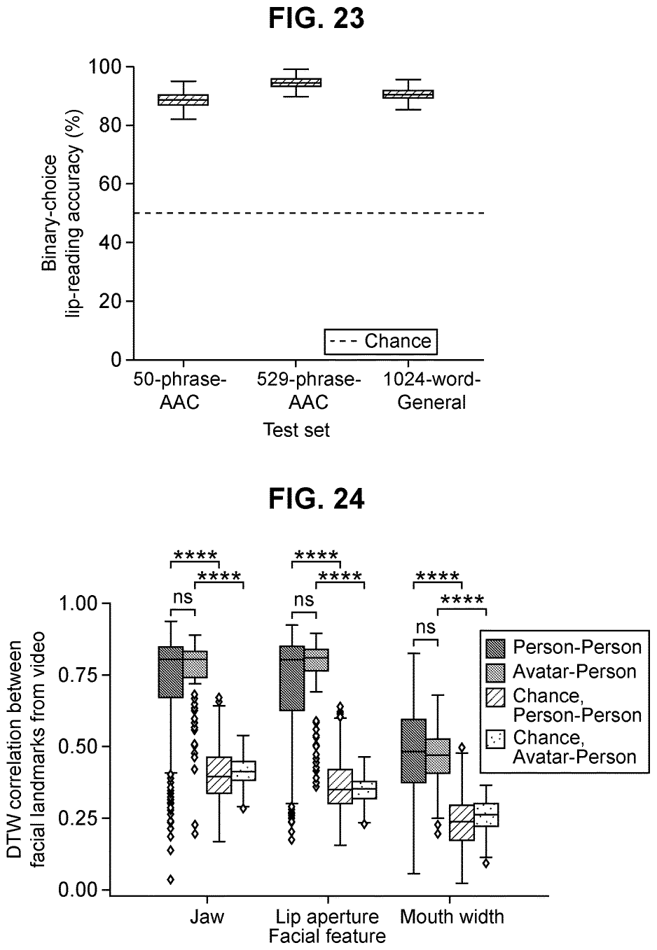

Avatar animations rendered in real time using the acoustic approach also exhibited strong correlations with reference articulatory gestures (FIG. 22; Table 8), high perceptual accuracy (FIG. 23), and visual facial-landmark trajectories that were closely correlated with healthy-speaker trajectories (FIG. 24; Table 9). These findings emphasize the strong performance of the speech-synthesis neural decoder when used with the commercial speech-to-gesture rendering system, although this approach cannot be used to generate meaningful facial gestures in the absence of a speech waveform.

TABLE 8
Comparisons for acoustic approach to avatar animation.

| | | | | Corrected |
|---|---|---|---|---|
| Articulator | Dataset 1 | Dataset 2 | Statistic¹ | P-value² |
| Lower lip push | 1024-word-General | 1024-word-General Chance | 0.00e+00 | 3.05e−05 |
| Lower lip push | 50-phrase-AAC | 50-phrase-AAC Chance | 0.00e+00 | 9.77e−04 |
| Lower lip push | 529-phrase-AAC | 529-phrase-AAC Chance | 0.00e+00 | 9.77e−04 |
| Pinching | 1024-word-General | 1024-word-General Chance | 6.00e+00 | 3.05e−05 |
| Pinching | 50-phrase-AAC | 50-phrase-AAC Chance | 0.00e+00 | 9.77e−04 |
| Pinching | 529-phrase-AAC | 529-phrase-AAC Chance | 0.00e+00 | 9.77e−04 |
| Upper lip pull | 1024-word-General | 1024-word-General Chance | 0.00e+00 | 3.05e−05 |
| Upper lip pull | 50-phrase-AAC | 50-phrase-AAC Chance | 0.00e+00 | 9.77e−04 |
| Upper lip pull | 529-phrase-AAC | 529-phrase-AAC Chance | 0.00e+00 | 9.77e−04 |
| Lip flare | 1024-word-General | 1024-word-General Chance | 0.00e+00 | 3.05e−05 |
| Lip flare | 50-phrase-AAC | 50-phrase-AAC Chance | 0.00e+00 | 9.77e−04 |
| Lip flare | 529-phrase-AAC | 529-phrase-AAC Chance | 0.00e+00 | 9.77e−04 |
| Lower lip pull | 1024-word-General | 1024-word-General Chance | 0.00e+00 | 3.05e−05 |
| Lower lip pull | 50-phrase-AAC | 50-phrase-AAC Chance | 0.00e+00 | 9.77e−04 |
| Lower lip pull | 529-phrase-AAC | 529-phrase-AAC Chance | 0.00e+00 | 9.77e−04 |
| Jaw opening | 1024-word-General | 1024-word-General Chance | 0.00e+00 | 3.05e−05 |
| Jaw opening | 50-phrase-AAC | 50-phrase-AAC Chance | 0.00e+00 | 9.77e−04 |
| Jaw opening | 529-phrase-AAC | 529-phrase-AAC Chance | 0.00e+00 | 9.77e−04 |
| Tongue body raise | 1024-word-General | 1024-word-General Chance | 0.00e+00 | 3.05e−05 |
| Tongue body raise | 50-phrase-AAC | 50-phrase-AAC Chance | 0.00e+00 | 9.77e−04 |
| Tongue body raise | 529-phrase-AAC | 529-phrase-AAC Chance | 0.00e+00 | 9.77e−04 |
| Retraction | 1024-word-General | 1024-word-General Chance | 0.00e+00 | 3.05e−05 |
| Retraction | 50-phrase-AAC | 50-phrase-AAC Chance | 0.00e+00 | 9.77e−04 |
| Retraction | 529-phrase-AAC | 529-phrase-AAC Chance | 0.00e+00 | 9.77e−04 |
| Nostril flare | 1024-word-General | 1024-word-General Chance | 0.00e+00 | 3.05e−05 |
| Nostril flare | 50-phrase-AAC | 50-phrase-AAC Chance | 0.00e+00 | 9.77e−04 |
| Nostril flare | 529-phrase-AAC | 529-phrase-AAC Chance | 0.00e+00 | 9.77e−04 |
| Tongue tip raise | 1024-word-General | 1024-word-General Chance | 0.00e+00 | 3.05e−05 |
| Tongue tip raise | 50-phrase-AAC | 50-phrase-AAC Chance | 0.00e+00 | 9.77e−04 |
| Tongue tip raise | 529-phrase-AAC | 529-phrase-AAC Chance | 0.00e+00 | 9.77e−04 |
| Compression | 1024-word-General | 1024-word-General Chance | 0.00e+00 | 3.05e−05 |
| Compression | 50-phrase-AAC | 50-phrase-AAC Chance | 0.00e+00 | 9.77e−04 |
| Compression | 529-phrase-AAC | 529-phrase-AAC Chance | 0.00e+00 | 9.77e−04 |
| Tongue advance | 1024-word-General | 1024-word-General Chance | 0.00e+00 | 3.05e−05 |
| Tongue advance | 50-phrase-AAC | 50-phrase-AAC Chance | 0.00e+00 | 9.77e−04 |
| Tongue advance | 529-phrase-AAC | 529-phrase-AAC Chance | 0.00e+00 | 9.77e−04 |
| Tongue retraction | 1024-word-General | 1024-word-General Chance | 1.00e+00 | 3.05e−05 |
| Tongue retraction | 50-phrase-AAC | 50-phrase-AAC Chance | 0.00e+00 | 9.77e−04 |
| Tongue retraction | 529-phrase-AAC | 529-phrase-AAC Chance | 0.00e+00 | 9.77e−04 |
| Adduction | 1024-word-General | 1024-word-General Chance | 0.00e+00 | 3.05e−05 |
| Adduction | 50-phrase-AAC | 50-phrase-AAC Chance | 0.00e+00 | 9.77e−04 |
| Adduction | 529-phrase-AAC | 529-phrase-AAC Chance | 0.00e+00 | 9.77e−04 |
| Lower lip tuck | 1024-word-General | 1024-word-General Chance | 0.00e+00 | 3.05e−05 |
| Lower lip tuck | 50-phrase-AAC | 50-phrase-AAC Chance | 0.00e+00 | 9.77e−04 |
| Lower lip tuck | 529-phrase-AAC | 529-phrase-AAC Chance | 0.00e+00 | 9.77e−04 |
| Rounding | 1024-word-General | 1024-word-General Chance | 0.00e+00 | 3.05e−05 |
| Rounding | 50-phrase-AAC | 50-phrase-AAC Chance | 0.00e+00 | 9.77e−04 |
| Rounding | 529-phrase-AAC | 529-phrase-AAC Chance | 0.00e+00 | 9.77e−04 |

¹Each comparison is a two-sided Wilcoxon signed-rank test between correlations from real-time decoded articulatory gestures (generated using the acoustic approach) and reference gestures across n = 20 pseudo-blocks for the 1024-word-General sentence set and n = 15 pseudo-blocks for the 50-phrase-AAC and 529-phrase-AAC sentence sets.
²16-way Holm-Bonferroni correction for multiple comparisons (corresponding to the 16 gesture categories).

TABLE 9
Comparisons for dlib traces with the acoustic approach.

| | | | | Corrected |
|---|---|---|---|---|
| Articulator | Dataset 1 | Dataset 2 | Statistic¹ | P-value² |
| Jaw | Avatar-Person | Person-Person | 4.09e+04 | 8.36e−01 |
| Jaw | Avatar-Person | Chance, Avatar- | 2.30e+04 | 2.00e−49 |
| | | Person | | |
| Jaw | Person-Person | Chance, Person- | 2.56e+05 | 1.62e−114 |
| | | Person | | |
| Lip aperture | Avatar-Person | Person-Person | 4.23e+04 | 7.88e−01 |
| Lip aperture | Avatar-Person | Chance, Avatar- | 2.30e+04 | 2.36e−49 |
| | | Person | | |
| Lip aperture | Person-Person | Chance, Person- | 2.58e+05 | 3.95e−119 |
| | | Person | | |
| Mouth width | Avatar-Person | Person-Person | 3.64e+04 | 1.89e−01 |
| Mouth width | Avatar-Person | Chance, Avatar- | 2.22e+04 | 1.67e−43 |
| | | Person | | |
| Mouth width | Person-Person | Chance, Person- | 2.58e+05 | 2.40e−118 |
| | | Person | | |

¹Each comparison is a two-sided Mann-Whitney U-test between dlib facial-landmark feature correlations for the avatar (decoded using the acoustic approach) and healthy-speaker videos, with n = 19 pseudo-blocks for each of the 8 speakers (yielding 152 data points for avatar-person comparisons) and n = 19 pseudo-blocks for each of the 28 combinations of pairs of speakers (yielding 532 data points for person-person comparisons).
²9-way Hohn-Bonferroni correction for multiple comparisons.

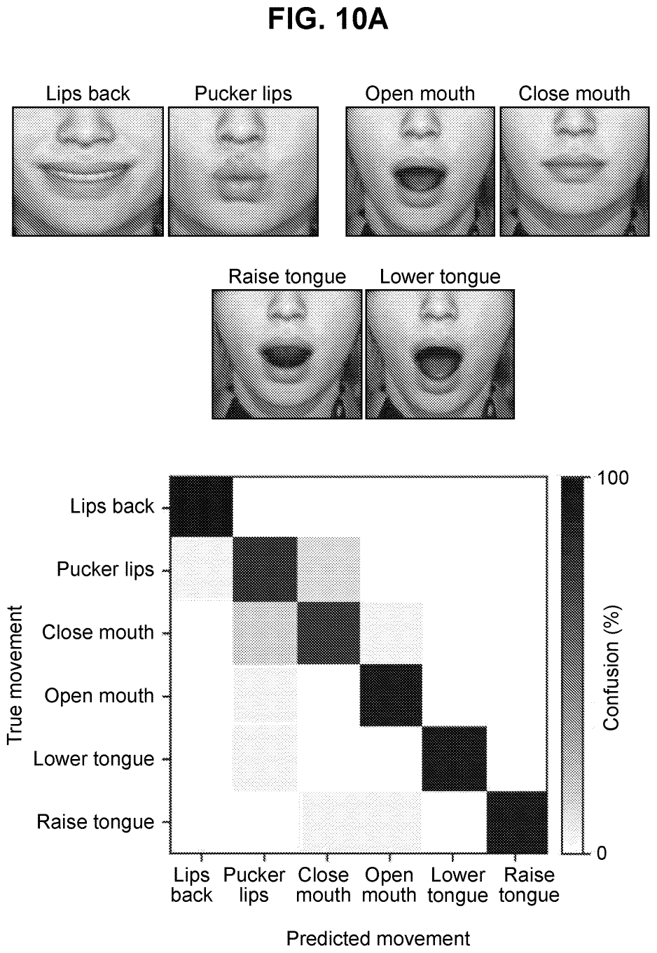

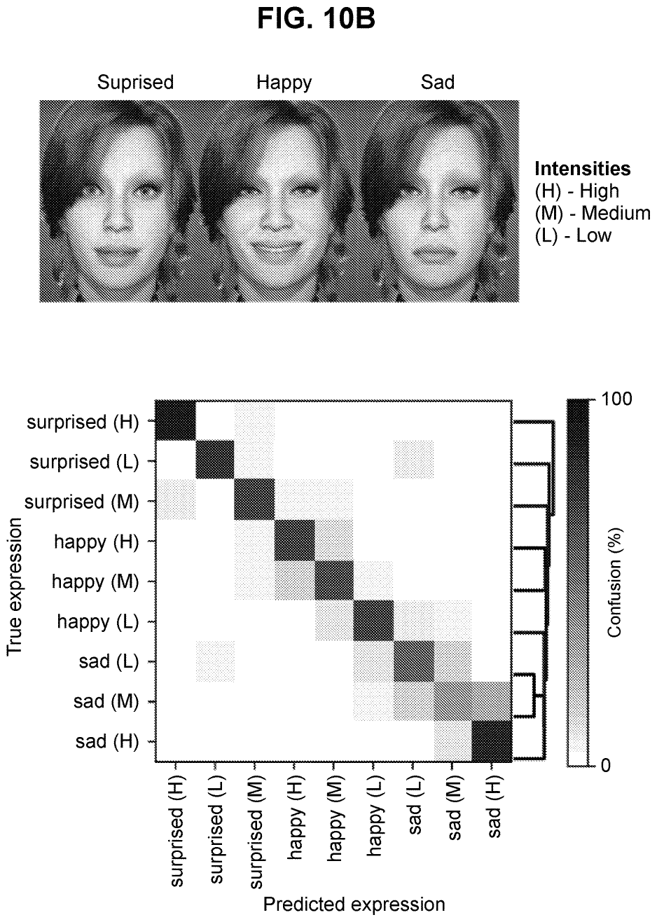

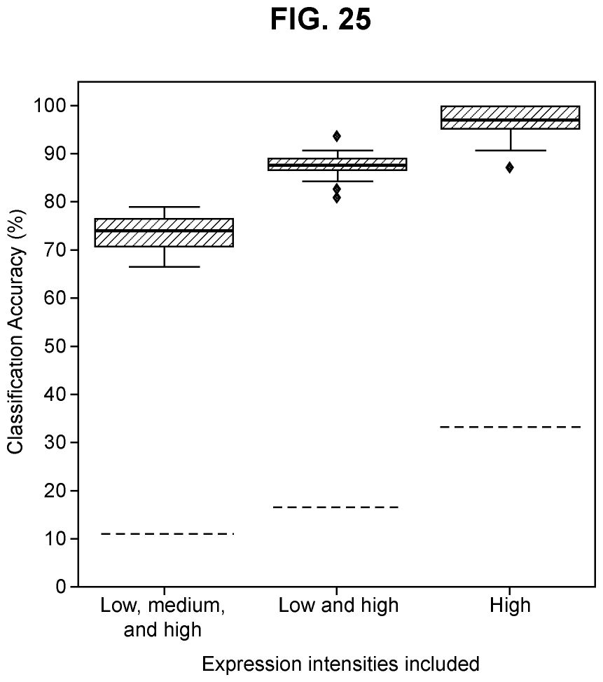

In addition to articulatory gestures to visually accompany synthesized speech, a fully embodying avatar BCI would also enable the user to portray non-speech orofacial gestures, including movements of particular orofacial muscles and expressions that convey emotion. To this end, neural data was collected from the participant as she performed two additional tasks: an articulatory-movement task and an emotional-expression task. In the articulatory-movement task, the participant attempted to produce 6 orofacial movements: jaw opening, lip puckering, lip retraction (smiling), tongue raising, tongue lowering, and rest (idle with mouth closed). In the emotional-expression task, the participant attempted to produce 3 types of expressions—happy, sad, and surprised—with either low, medium, or high intensity, resulting in 9 unique expressions in total. Offline, for the articulatory-movement task a small feed-forward neural-network model was trained to learn the mapping between the ECoG features and each of the targets. For the articulatory-movement task, a median classification accuracy of 87.8% (99% CI [85.1, 90.5]; across n=10 cross-validation folds; FIG. 10A) was observed when classifying between the 6 movements. For the emotional-expression task, a small RNN to was trained learn the mapping between ECoG features and each of the expression targets. A median classification accuracy of 74.0% (99% CI [70.8, 77.1]; across n=15 cross-validation folds; FIG. 10B) was observed when classifying between the 9 possible expressions and a median classification accuracy of 96.9% (99% CI [93.8,100]) when only considering the classifier's outputs for the strong-intensity versions of the 3 expression types (FIG. 25). In separate, qualitative task blocks, it was shown that the participant could control the avatar BCI to portray the articulatory movements (Video 6) and strong-intensity emotional expressions (Video 7), illustrating the potential of multimodal communication BCIs to restore the ability to express meaningful orofacial gestures.

FIG. 8, FIGS. 9A to 9B, and FIGS. 10A to 10B depict direct decoding of orofacial articulatory gestures from neural activity to drive an avatar. FIG. 8 is a schematic diagram of the avatar decoding algorithm. Offline, a bidirectional recurrent neural network (RNN) decodes neural activity recorded during attempts to silently speak into discretized articulatory gestures (quantized via a vector quantized variational autoencoder, abbreviated VQ-VAE). A convolutional neural network de-quantizer (VQ-VAE decoder) is then applied to generate the final predicted gestures, which are then passed through a pre-trained gesture-animation model to animate the avatar in a virtual environment. FIG. 9A binary perceptual accuracies from human evaluators on avatar animations generated from neural activity. Evaluators see the decoded avatar animations (with no accompanying audio) and, for each one, choose between the correct reference text target and a randomly selected incorrect text string. FIG. 9B correlations for jaw, lip, and mouth-width movements between decoded avatar renderings and videos of real human speakers on the 1024-word-General sentence set across all pseudo-blocks for each comparison (n=152 for avatar-person comparison, n=532 for person-person comparisons; ****P<0.0001, Mann-Whitney U-test with 9-way Holm-Bonferroni correction; p-values and U-statistics in Table 7). A facial-landmark detector (dlib) was used to measure orofacial movements from the videos. FIG. 10A top: snapshots of avatar animations of 6 non-speech articulatory movements in the articulatory-movement task. Bottom: confusion matrix depicting classification accuracy across the movements. The classifier was trained to predict which movement the participant was attempting from her neural activity, and the prediction was used to animate the avatar. FIG. 10B top: snapshots of avatar animations of 3 non-speech emotional expressions in the emotional-expression task. Bottom: confusion matrix depicting classification accuracy across 3 intensity levels (high, medium, and low) of the 3 expressions, ordered via hierarchical agglomerative clustering on the confusion values. The classifier was trained to predict which expression the participant was attempting from her neural activity, and the prediction was used to animate the avatar.

FIG. 20 provides examples of directly decoded avatar articulatory gestures. Examples of directly decoded articulatory gestures (colored) compared with reference articulatory gestures (black). Examples were taken from the 50-phrase-AAC sentence set. Dynamic time warping [86] was applied to align traces prior to plotting and computation of Pearson r correlation, which is displayed to the right of each gesture. Reference articulatory gestures were computed using the speech-to-gesture technology from SG Com.

FIG. 21 provides correlations of directly decoded avatar articulatory gestures with reference articulatory gestures. Pearson correlation (R) of decoded articulatory gestures with reference articulatory gestures using the direct decoding approach after applying dynamic time warping using fast-dtw [86] to align the reference and decoded gestures, since the participant never heard the reference waveform used to derive reference gestures. Chance values are derived by shuffling the neural data temporally then feeding it through our decoding pipeline. The resulting traces are then warped using fast-dtw and compared with reference traces. Correlations were significantly above chance for all comparisons except comparisons of nostril flare for all sentence sets and pinching for the 1024-word-General sentence set, two-sided Wilcoxon Signed Rank test with 16-way HolmBonferroni correction across n=20 pseudo-blocks for the 1024-word-General sentence set, n=15 pseudo-blocks for AAC sets. See Supplementary Table 6 for all p-values and statistics. ****P<0.0001, ***P<0.001, **P<0.01.

FIG. 22 provides correlations between avatar articulatory gestures with reference articulatory gestures using the acoustic approach. Pearson correlation (R) of decoded articulatory gestures with reference articulatory gestures during acoustic approach after applying dynamic time warping using fast-dtw [86] to align the reference and decoded gestures, since the participant never heard the reference waveform used to derive reference gestures. Chance values are derived by shuffling the neural data temporally then feeding it through our decoding pipeline. The resulting traces are then warped using fast-dtw and compared with reference traces. Correlations were significantly above chance for all comparisons, two-sided Wilcoxon Signed Rank test with 16-way Holm-Bonferroni correction across n=20 pseudo-blocks for the 1024-word-General sentence set, n=15 pseudo-blocks for AAC sets. See Supplementary Table 8 for all p-values and statistics. ****P<0.0001, ***P<0.001, **P<0.005 *<0.01.

FIG. 23 illustrates perceptual accuracy for the avatar using the acoustic approach or, more specifically, binary perceptual accuracy from human evaluation of silent videos extracted from the audio-visual synthesis task. The median bootstrapped accuracy across six evaluators was used to represent the final accuracy for each sentence. Median accuracies of 88.7% (99% CI [81.7, 94.0]), 94.3% (99% CI [89.7, 98.3]), and 90.5% (99% CI [85.5, 95.0]) were obtained across 150 trials of the 50-phrase-AAC sentence set, 150 trials of the 529-phrase-AAC sentence set, and 200 trials of the 1024-word-General sentence set, respectively.

FIG. 24 provides correlations of facial landmarks using the acoustic approach for avatar decoding the 1024-word-General sentence set or, more specifically, correlations of facial landmark trajectories (extracted using dlib) within healthy speakers and between healthy speakers and the avatar. The avatar was animated using real-time testing blocks for audio-visual synthesis where the decoded acoustic waveform was used with the acoustic speech-to-gesture approach for animation. 8 healthy speakers spoke the same sentences. Correlations were measured using the pearson correlation after applying dynamic time warping. Similar results were observed as for direct decoding with the 1024-word-General sentence set (FIG. 9B), where correlations between the avatar and a healthy speaker were comparable to correlations between two healthy speakers. Mean correlations were 0.801 (99% CI [0.789, 0.814]), 0.808 (99% CI [0.793, 0.815]), and 0.469 (99% CI [0.443, 0.494]) for jaw, lip aperture, and mouth width, respectively. These results were significantly better than chance (see Table 9 for statistics and p-values). Interestingly, the correlations between person-person and avatar-person were not significantly different with this approach, demonstrating a promising path to avatar animation, but more limited than directly decoding articulatory representations.

FIG. 25 provides classification results of emotional expressions. Full 15-fold cross validation classification accuracy for emotional expressions across different subsets of intensities in the emotional-expression task are depicted. Chance for each paradigm is indicated by the dashed black line. Box plots consist of (n=15) cross validation folds. Median cross-validation fold accuracy was 74.0% (99% CI [70.8, 77.1]) for all low, medium, and high intensity expressions, 87.5% (99% CI [84.4, 89.1]) for all low and high intensity expressions, and 96.9% (99% CI [93.8,100]) for all high intensity expressions.

### 1.3.4. Articulatory Representations Driving Speech Decoding

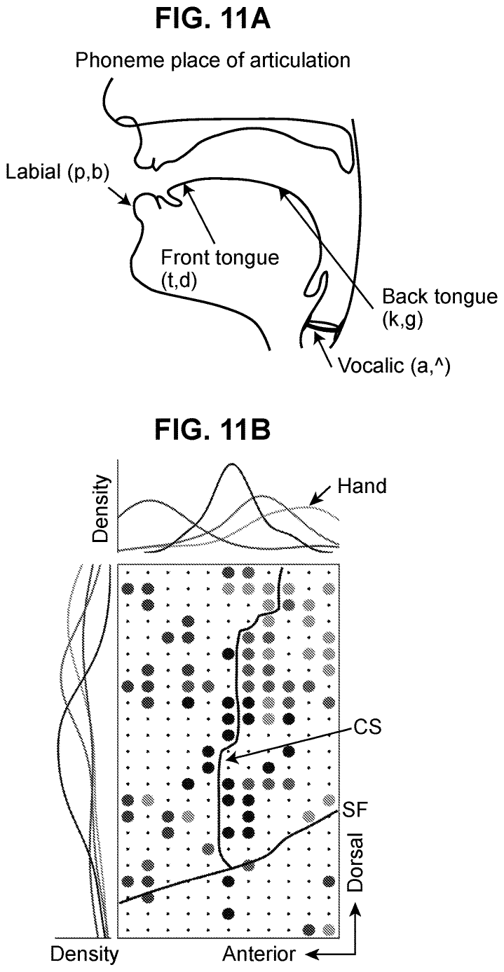

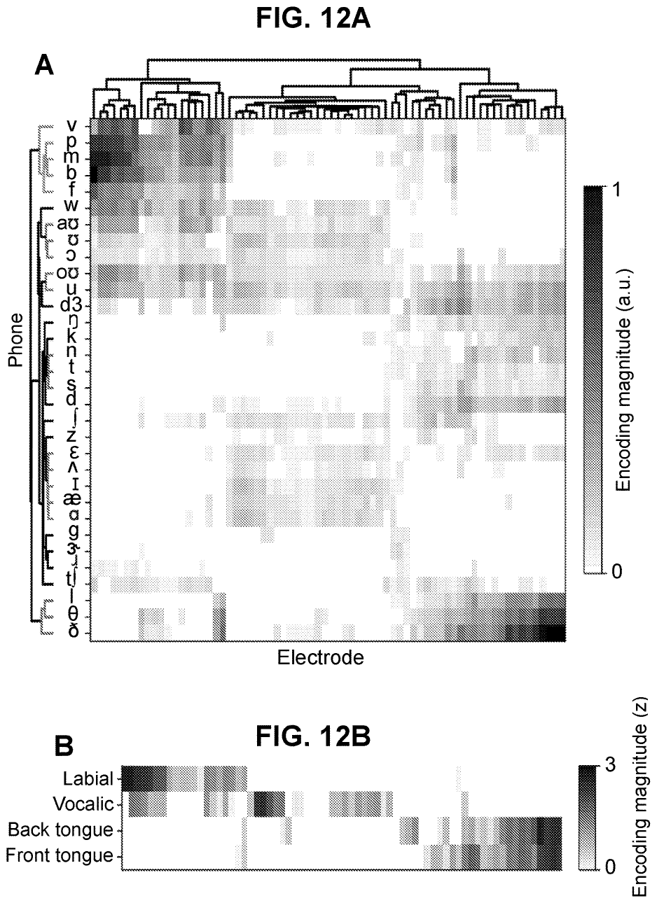

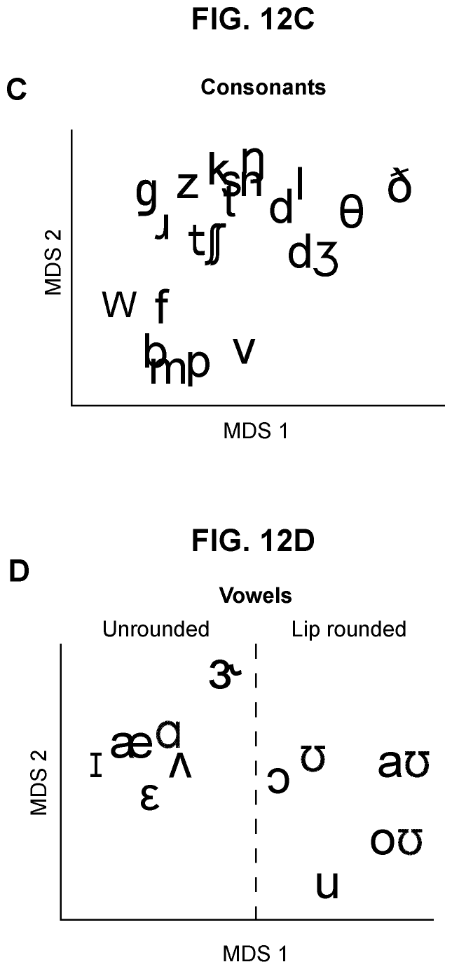

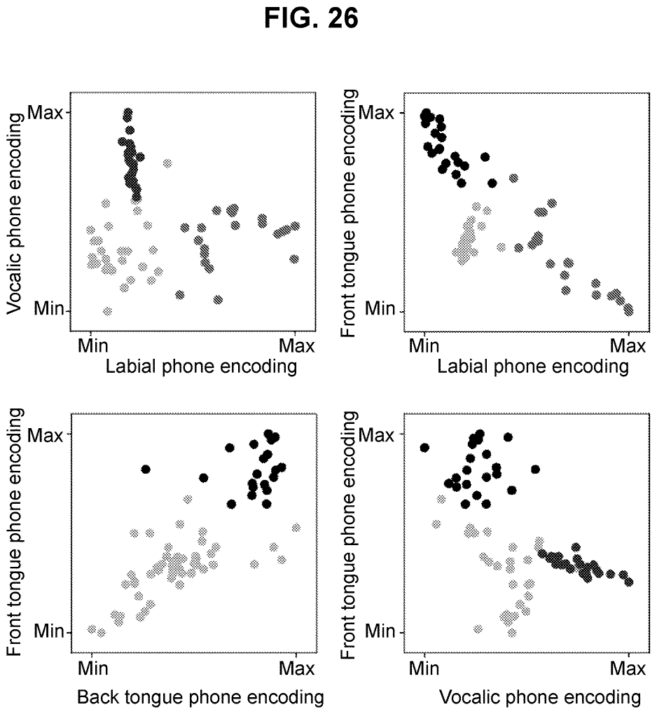

In healthy speakers, neural representations in the sensorimotor cortex (SMC, comprising the precentral and postcentral gyri) encode articulatory movements of the orofacial musculature [26], [26], [31]. With the implanted electrode array centered over the SMC of the participant, it was asked if articulatory representations persisting after paralysis underlied speech-decoding performance. To assess this, a linear temporal receptive-field encoding model was first fit to predict high-gamma activity for each electrode from the phone probabilities computed by the text decoder during the 1024-word-General text task condition. For each speech-activated electrode, the maximum encoding weight was calculated for each phone. This yielded a phonetic-tuning space in which each electrode had an associated vector of relative phone-encoding weights. Within this space, it was determined if phone clustering was organized by the primary orofacial articulator associated with each phone (place of articulation, POA; FIG. 11A), which has been shown in prior studies with healthy speakers [24] [25]. Phones were parceled into four POA categories: labial, vocalic, back tongue, and front tongue. Hierarchical clustering of phones in this tuning space revealed a clear grouping by POA (P<0.0001 compared to chance, one-tailed permutation test; FIG. 12A). A variety of tunings were observed across the electrodes, with some electrodes exhibiting tuning to single POA categories and others to multiple categories (such as both front-tongue and back-tongue phones or both labial and vocalic phones; FIG. 12B; FIG. 26). Multidimensional scaling was used to visualize the phonetic tunings in a two-dimensional space, revealing separability between labial and non-labial consonants (FIG. 12C) and between lip-rounded and non-lip-rounded vowels (FIG. 12D).

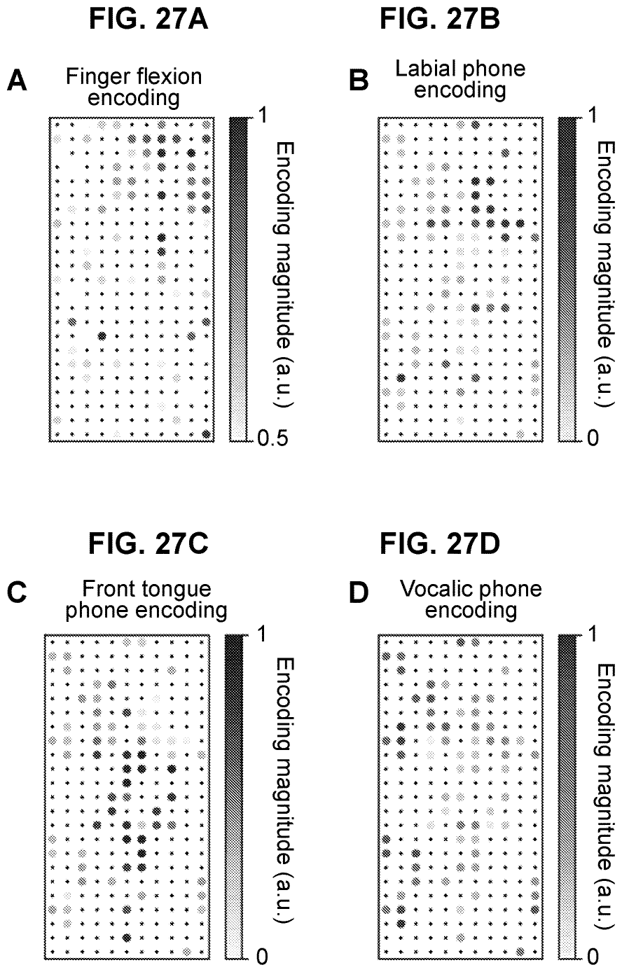

Next, it was investigated whether these articulatory representations were arranged somatotopically (with ordered regions of cortex preferring single articulators), which is observed in healthy speakers [25]. Because the dorsal-posterior corner of the ECoG array provided coverage of the hand cortex, how neural activation patterns related to attempted hand movements fit into the somatotopic map was also assessed, using data collected during the NATO-motor task containing four finger flexion targets (either thumb or simultaneous index- and middle-finger flexion for each hand). The grid locations of the electrodes were visualized that most strongly encoded the vocalic, front-tongue, and labial phones as well attempted hand movement (the top 30% of electrodes were included for each condition; FIG. 11B; see FIG. 27 for full electrode encoding maps). Kernel density estimates revealed a somatotopic map with encoding of attempted hand movements, labial phones, and front-tongue phones organized along a dorsal-ventral axis. The relatively anterior localization of the vocalic cluster in the precentral gyrus is likely associated with the laryngeal motor cortex, consistent with previous investigations in healthy speakers [25] [26] [31] [32].

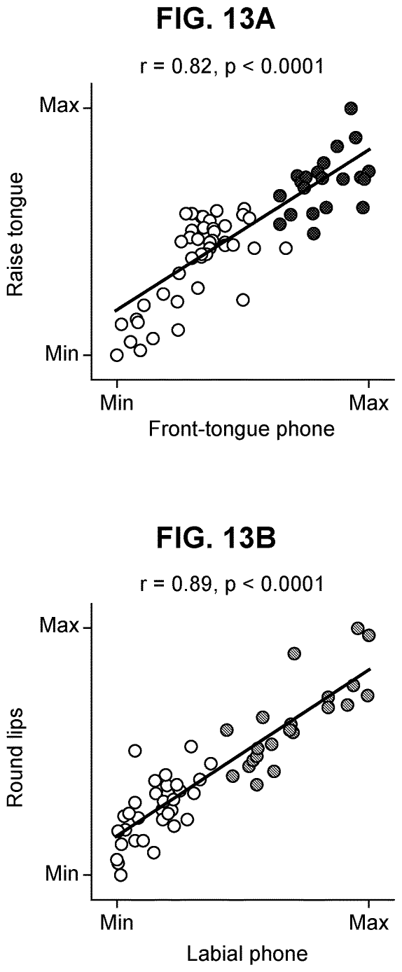

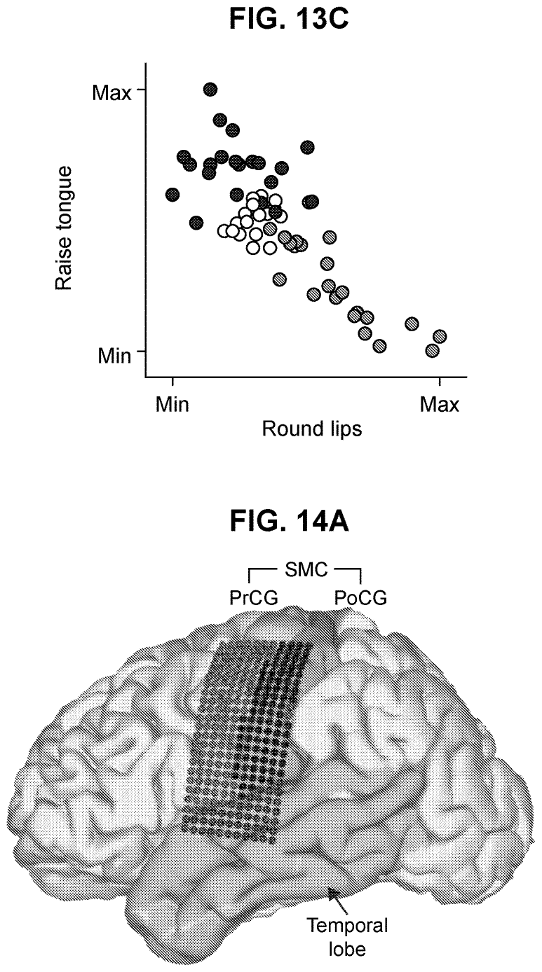

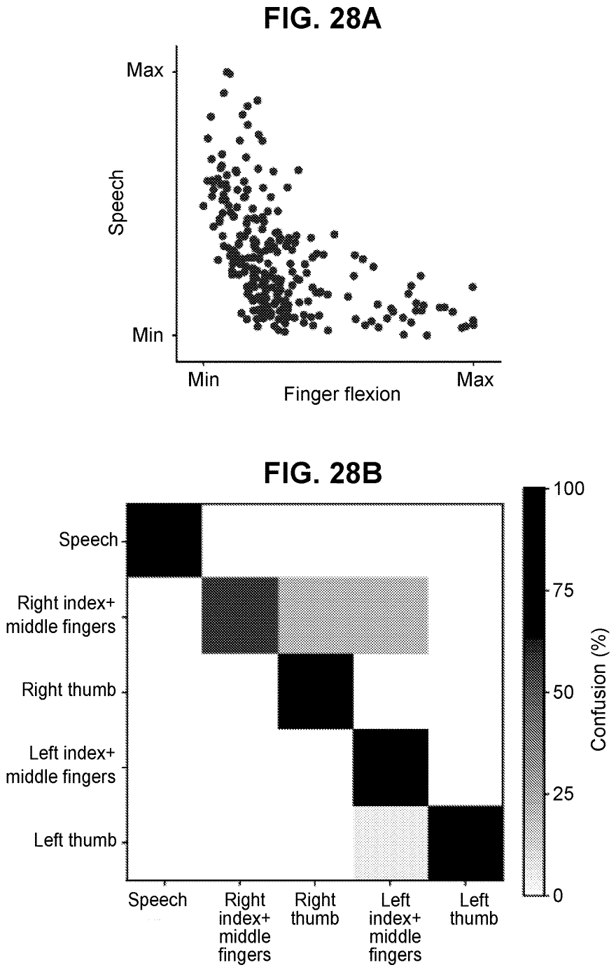

Finally, whether the same electrodes that encoded POA categories during silent speech attempts also encoded non-speech articulatory-movement attempts was assessed. Using the previously computed phonetic encodings and high-gamma activity recorded during the articulatory movement task, a positive correlation was found between front-tongue phonetic encoding and high-gamma activation magnitude during attempts to raise the tongue (P<0.0001, r=0.82, ordinary least squares regression; FIG. 13A). A positive correlation was also observed between labial phonetic tuning and activation magnitude during attempts to pucker the lips (P<0.0001, r=0.89, ordinary least squares regression; FIG. 13B). Although most electrodes were selective to either lip or tongue movements, others were activated by both (FIG. 13C). During the NATO-motor task, electrodes encoding attempted finger flexions were largely orthogonal to those encoding NATO code words, which helped to enable accurate neural discrimination between the four finger-flexion targets and the silent-speech targets (the model correctly classified 569 out of 570 test trials as either finger flexion or silent speech; FIG. 28).

FIGS. 11A to 11B, FIGS. 12A to 12D, and FIGS. 13A to 13C depict articulatory encodings driving speech decoding. FIG. 11A is a mid-sagittal schematic of the vocal tract with phone place of articulation (POA) features labeled. FIG. 12A illustrates phone-encoding vectors for each electrode computed by a temporal receptive-field model on neural activity recorded during attempts to silently say sentences from the 1024-word-General set, organized by unsupervised hierarchical clustering. FIG. 12B provides Z-scored POA encodings for each electrode, computed by averaging across positive phone encodings within each POA category. Z values are clipped at 0. FIGS. 12C to 12D provide projection of consonant (FIG. 12C) and vowel (FIG. 12D) phone encodings into a two-dimensional space via multidimensional scaling (MDS). FIG. 11B bottom-right: visualization of the locations of electrodes with the greatest encoding weights for labial, front-tongue, and vocalic phones on the electrocorticography array. The electrodes that most strongly encoded finger flexion during the NATO-motor task are also included. Only the top 30% of electrodes within each condition are shown, and the strongest tuning was used for categorization if an electrode was in the top 30% for multiple conditions. Black lines denote the central sulcus (CS) and sylvian fissure (SF). Top and bottom-left: The spatial electrode distributions for each condition along the anterior-posterior and ventral-dorsal axes, respectively. FIGS. 13A to 13C provide electrode-tuning comparisons between front-tongue phone encoding and tongue-raising attempts (FIG. 13A; r=0.82, P<0.0001, ordinary least squares regression), labial phone encoding and lip-puckering attempts (FIG. 13B; r=0.89, P<0.0001, ordinary least squares regression), and tongue-raising and lip-rounding attempts (FIG. 13C). Non-phonetic tunings were computed from neural activations during the articulatory-movement task. Each plot depicts the same electrodes encoding front-tongue and labial phones (from FIG. 11B) as blue and orange dots, respectively; all other electrodes are shown as gray dots.

FIG. 26 illustrates cross-phone place-of-articulation encoding. For each electrode included in FIG. 11B the relationship between encoding of phone place of articulation (POA) categories was visualized. The electrode color represents the top 30% of encoding electrodes for a given POA (as in FIG. 11B).

FIGS. 27A to 27D demonstrate spatial distribution of electrode tuning to articulatory features. Shown are normalized [0-1] encoding weights across electrodes for (A) hand finger flexion, (B) labial phones, (C) front tongue phones, and (D) vocalic phones. For (B) to (D) data is shown for all speech responsive electrodes with encoding r>0.2. For (A) data is shown for the top 50% of finger-flexion encoding electrodes.

FIGS. 28A to 28B demonstrate that attempted finger flexion and speech are largely encoded orthogonally. (A) for each electrode, the normalized [0,1] encoding in response to attempted production of NATO code-words is plotted against attempted finger flexion in the NATO-motor task. (B) confusion matrix from the NATO-motor task, showing minimal confusion between hand and speech targets.

### 1.3.5. Effects of Limiting Coverage, Density, and Features

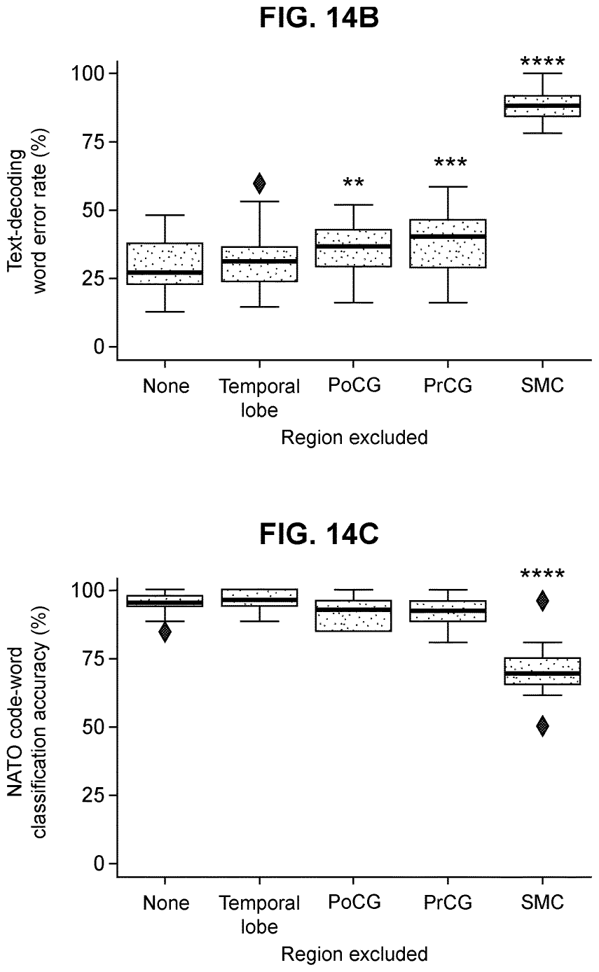

Prior work has shown that brain-based speech-decoding performance is heavily influenced by electrode coverage, electrode density, and feature extraction [2] [3] [11] [12] [33-35]. To assess how these factors affected the results, the impact that several canonical speech production regions had on decoding performance were first quantified [36]. Offline leave-one-region-out evaluations were performed in which all electrodes in one of the following anatomical regions were excluded during training and testing of text decoding RNNs with the 1024-word-general set: precentral gyrus, postcentral gyrus, sensorimotor cortex (SMC; precentral and postcentral gyri), and the temporal lobe (FIG. 14A). Excluding electrodes from any region except the temporal lobe significantly increased WER for text decoding (FIG. 14B, P<0.005 for all, two-sided Wilcoxon signed-rank tests with 10-way Holm-Bonferroni correction; Table 10). For NATO code-word classification, only exclusion of all SMC electrodes significantly decreased classification accuracy (FIG. 14C; P<0.0001 for SMC exclusion, all other p>0.01, Wilcoxon signed-rank tests with 10-way Holm-Bonferroni correction). Exclusion of the SMC had the strongest effect on performance for both models (P<0.0001, two-sided Wilcoxon signed-rank tests with 10-way Holm-Bonferroni correction). No difference between exclusion of the precentral and postcentral gyrus was seen across decoding modalities (Table 10). Consistent with prior research, these results illustrate the importance of neural populations in the SMC for the speech-decoding models, informing effective surgical-implantation strategies for future clinical studies. These findings are provide evidence for motor movement specific states in the postcentral gyrus and highlight a level of redundancy in encoded speech content between the precentral and postcentral gyri, which could have favorable implications for chronic reliability with ECoG-based speech BCIs.

TABLE 10
Exclusion comparisons between anatomical regions.

| | Region | Region | | Corrected |
|---|---|---|---|---|
| Task¹ | excluded 1 | excluded 2 | Statistic² | P-value³ |
| Text WER | SMC | temporal lobe | 0.00e+00 | 5.96e−07 |
| Text WER | SMC | none | 0.00e+00 | 5.96e−07 |
| Text WER | SMC | postcentral | 0.00e+00 | 5.96e−07 |
| Text WER | SMC | precentral | 0.00e+00 | 5.96e−07 |
| Text WER | temporal | none | 8.90e+01 | 1.65e−01 |
| | lobe | | | |
| Text WER | temporal | postcentral | 9.10e+01 | 1.65e−01 |
| | lobe | | | |
| Text WER | temporal | precentral | 7.30e+01 | 5.89e−02 |
| | lobe | | | |
| Text WER | none | postcentral | 2.40e+01 | 2.63e−03 |
| Text WER | none | precentral | 1.10e+01 | 4.29e−04 |
| Text WER | postcentral | precentral | 9.50e+01 | 3.06e−01 |
| NATO code-word | SMC | temporal lobe | 0.00e+00 | 3.81e−05 |
| accuracy | | | | |
| NATO code-word | SMC | none | 0.00e+00 | 3.81e−05 |
| accuracy | | | | |
| NATO code-word | SMC | postcentral | 0.00e+00 | 3.81e−05 |
| accuracy | | | | |
| NATO code-word | SMC | precentral | 0.00e+00 | 3.81e−05 |
| accuracy | | | | |
| NATO code-word | temporal | none | 1.50e+01 | 7.15e−01 |
| accuracy | lobe | | | |
| NATO code-word | temporal | postcentral | 8.00e+00 | 1.35e−02 |
| accuracy | lobe | | | |
| NATO code-word | temporal | precentral | 8.50e+00 | 2.01e−02 |
| accuracy | lobe | | | |
| NATO code-word | none | postcentral | 7.00e+00 | 3.21e−02 |
| accuracy | | | | |
| NATO code-word | none | precentral | 5.00e+00 | 1.25e−02 |
| accuracy | | | | |
| NATO code-word | postcentral | precentral | 5.40e+01 | 7.28e−01 |
| accuracy | | | | |

¹Abbreviations: Word error rate (WER); Sensorimotor cortex (SMC)
²Each comparison is a two-sided Wilcoxon signed-rank test between n = 25 text WER pseudoblocks or n = 19 NATO blocks
³10-way Holm-Bonferroni correction for multiple comparisons.

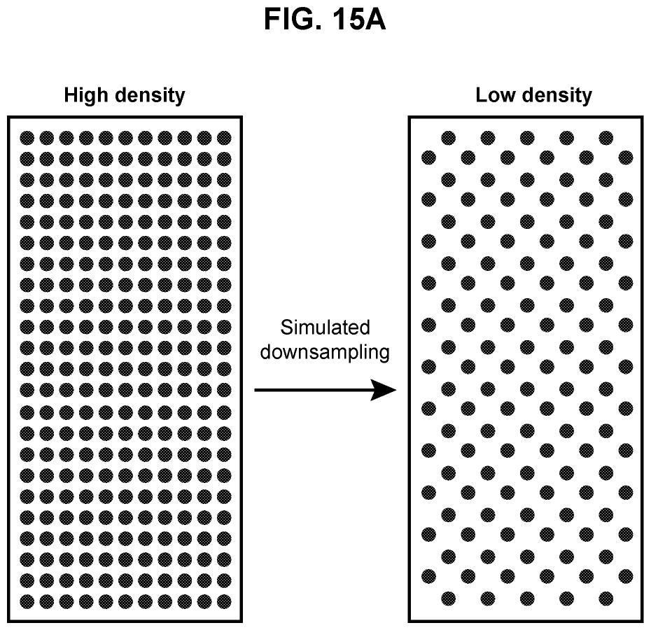

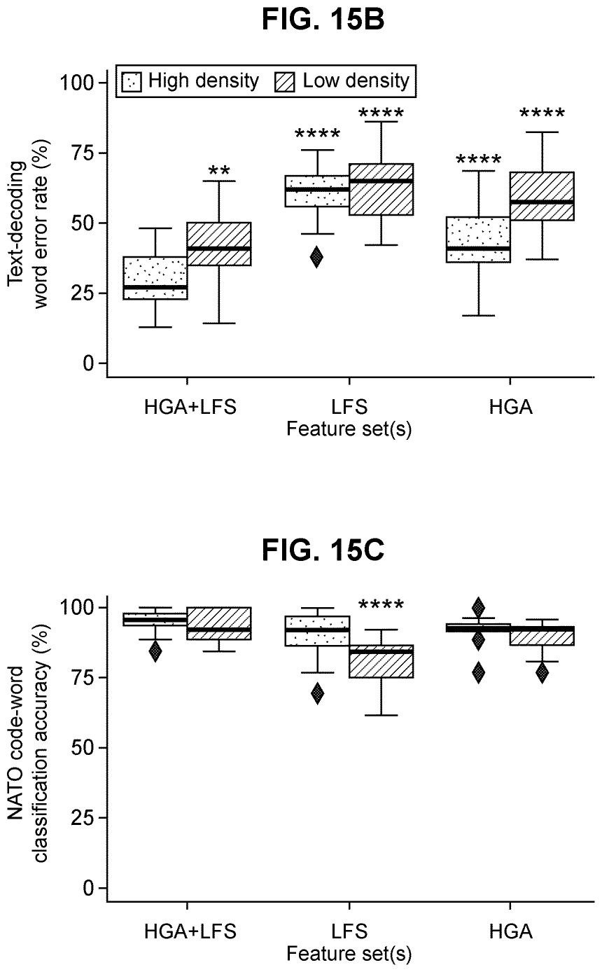

To measure the impact that electrode density and feature selection had on decoding performance, additional offline analyses was performed with the same three models. First, a lower-density ECoG array was simulated using checkerboard downsampling, yielding 127 electrodes at 4.24-mm center-to-center spacing from the original 253 electrodes at 3-mm spacing (FIG. 15A). Then, the decoding models were trained and tested using each density (either high or low) and either the original feature set (high-gamma activity and low-frequency signals; HGA+LFS) or one of two limited feature sets containing only HGA or only LFS. Using low-density sampling or a limited feature set significantly increased the WER for text decoding (FIG. 15B) with the 1024-word-General sentence set (all P<0.005, two-sided Wilcoxon signed-rank tests with 15-way Holm-Bonferroni correction; Table 11). For NATO code-word classification, only the combination of low-density sampling and the LFS-only feature set resulted in significantly worse classification accuracy, although the accuracy decrease was small (FIG. 15C; P<0.01 for low-density coverage and LFS-only features, all other p>0.01, two-sided Wilcoxon signed-rank tests with 15-way Holm-Bonferroni correction). Generally, using only HGA outperformed only LFS (Table 11). These findings are also consistent with prior work and further emphasize the benefits of having high-density coverage and neural features from a broad range of frequencies, informing future array design and signal-processing pipelines for speech-decoding applications.

TABLE 11
Exclusion comparisons between feature set (s) and electrode density.

| [symbol] | [symbol] | [symbol] | [symbol] | [symbol] | [symbol] | [symbol] |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

[symbol]

indicates data missing or illegible when filed

FIGS. 14A to 14C and FIGS. 15A to 15C depict effects of anatomical coverage, electrode density, and feature sets on decoding performance. FIG. 14A depicts MRI reconstruction of the participant's brain overlaid with the locations of implanted electrodes. Cortical regions and electrodes are colored according to anatomical region (PoCG: postcentral gyrus, PrCG: precentral gyrus, SMC: sensorimotor cortex). FIGS. 14B to 14C provide the effect of excluding each region during training and testing on text-decoding word error rates (FIG. 14B), and NATO code-word classification accuracies (FIG. 14C), computed using neural data as the participant attempted to silently say either sentences from the 1024-word-General set (FIG. 14B) or NATO code words with the NATO-motor task (FIG. 14C). Significance markers indicate comparisons against the None condition, which uses all electrodes. FIG. 15A is a visualization of the checkerboard-downsampling procedure used to simulate a low-density electrocorticography array with 127 electrodes instead of 253. FIGS. 15B to 15C provide the effect of modulating electrode density and feature set on text-decoding word error rates (FIG. 15B) and NATO code-word classification accuracies (FIG. 15C), computed using the same base data as in FIGS. 14B to 14C (HGA: high-gamma activity; LFS: low-frequency signals; HGA+LFS: both feature sets). Significance markers indicate comparisons against the full-model condition (high density with HGA+LFS). In FIGS. 14B to 14C and FIGS. 15B to 15C, *P<0.01, **P<0.005, ***P<0.001, ****P<0.0001, two-sided Wilcoxon signed-rank test with 10-way (FIGS. 14B to 14C) or 15-way (FIGS. 15B to 15C) Holm-Bonferroni correction (full comparisons for FIGS. 14B to 14C and FIGS. 15B to 15C are given in Tables 10 and 11, respectively). Distributions are over 25 pseudo-blocks for text decoding and 19 blocks for NATO code-word classification.

### 1.4. SUMMARY AND CONCLUSIONS

Faster, more accurate, and more natural communication are among the most desired needs from people who have lost the ability to speak after severe paralysis [1] [40-42]. Here, it is demonstrated that all of these needs can be addressed with a speech-neuroprosthetic device that decodes articulatory cortical activity into multiple output modalities, including text, speech audio synchronized with a facial avatar, and facial expressions.

During 14 days of data collection shortly after device implantation, text was decoded from vocabulary of over 1,000 words with a 25% word error rate at 77.6 words per minute as the participant attempted to silently say sentences that were not seen during model training, exceeding communication speeds of previous brain-computer interfaces (BCIs) by a factor of 3 or more [2] [3] [9] and expanding the vocabulary size of prior direct-speech BCI by a factor of 20 [2]. It is also shown for the first time that intelligible speech can be synthesized from the brain activity of a person with paralysis. Finally, a novel modality of BCI control was introduced in the form of a digital “talking face”—a personalized avatar capable of dynamic, realistic, and interpretable speech movements that can be synchronized with synthesized speech or used to express non-verbal facial gestures. Together, these results have surpassed an important threshold of performance, generalizability, and expressivity in order to provide practical benefits to people with speech loss.

The progress here was enabled by several important technical and conceptual innovations including, among other things: 1) Advances in the neural interface, providing denser and broader sampling of the distributed orofacial and vocal-tract representations across the entire lateral sensorimotor cortex; 2) Highly stable recordings from non-penetrating cortical-surface electrodes, enabling training and testing across days and weeks without requiring day-of recalibration; 3) Custom sequence-learning neural-decoding models, facilitating training using only task go cues for data segmentation and without any other alignment of neural activity and output features (which is essential for training in the setting of paralysis); 4) Self-supervised learning-derived discrete speech units, serving as effective intermediate representations for intelligible speech synthesis; and 5) Novel control of a virtual face from brain activity to accompany synthesized speech and convey facial expressions.

Studies have characterized how neural populations in the SMC precisely coordinate the articulatory movements that give rise to spoken speech [24-26] [44], but these studies involved participants who could speak. Here, persistent articulatory encoding in the SMC of the participant were found that are consistent with prior intact-speech characterizations despite over 18 years of anarthria, including hand and orofacial-motor somatotopy organized along a dorsal-ventral axis and phonetic tunings clustered by place of articulation. Together with measures of model contributions from different anatomical regions, these outcomes show that text-decoding and speech-synthesis performance was driven by articulatory representations in the SMC and not higher-level linguistic representations, such as semantics. These representations also drove perceptually salient digital animations of the orofacial articulators themselves, even when the participant was performing non-speech expressions and facial gestures. Furthermore, the presence of hand-motor somatotopy and minimal confusability between finger flexions and speech is promising for future BCIs that integrate speech and limb control, building on foundational studies in restoring upper limb function [45-47].

While decoders were able to be trained without the participant hearing or seeing the targets for synthesis and avatar, providing instantaneous closed-loop feedback during decoding

has the potential to improve user engagement, model performance, and neural entrainment [49]. Also, further advances in electrode interfaces and decoding algorithms should continue to improve accuracy and generalizability towards eventual clinical applications.

The ability to interface with evolving technology to communicate with family and friends, facilitate community involvement and occupational participation, and engage in virtual, internet-based social contexts (such as social media and metaverses) can vastly expand a person's access to meaningful interpersonal interactions and ultimately improve their quality of life [1] [41]. Here it is shown that BCIs can give this ability back to patients through highly personalizable audio-visual synthesis capable of restoring aspects of their personhood and identity.

### 1.5. MATERIALS & METHODS: OVERVIEW

### 1.5.1. Participant

The participant, who was 47 years old at time of enrollment into the study, was diagnosed with quadriplegia and anarthria by neurologists and a speech-language pathologist following a right-pontine infarct in 2005 (when the participant was 30 years old). During enrollment testing, she scored 29/30 on the Mini Mental State Exam and was only unable to achieve the final point because she could not physically draw a figure due to her paralysis. She can vocalize a small set of monosyllabic sounds, such as “ah” or “ooh”, but she is unable to articulate intelligible words. During clinical assessments, a speech-language pathologist prompted her to say 58 words and 10 phrases and also asked her to respond to 2 open-ended questions within a structured conversation. From the resulting audio and video transcriptions of her speech attempts, the speech-language pathologist measured her intelligibility to be 5% for the prompted words, 0% for the prompted sentences, and 0% for the open-ended responses. Functionally, she cannot use speech to communicate. Instead, she relies on a transparent letter board and a Tobii Dynavox for communication. She used her transparent letter board to provide informed consent to participate in this study and to allow her image to appear in demonstration videos. To sign the physical consent documents, she used her communication board to spell out “I consent” and directed her spouse to sign the documents on her behalf.

### 1.5.2. Neural Implant

The neural-implant device used in this study featured a high-density ECoG array (PMT) and a percutaneous pedestal connector (Blackrock Microsystems). The ECoG array consists of 253 disk-shaped electrodes arranged in a lattice formation with 3-mm center-to-center spacing. Each electrode has a 1-mm recording-contact diameter and a 2-mm overall diameter. The array was surgically implanted on the pial surface of the left hemisphere of the brain, covering regions associated with speech production and language perception, including the middle aspect of the superior and middle temporal gyri, the precentral gyrus, and the postcentral gyrus. The percutaneous pedestal connector, which was secured to the skull during the same operation, conducts electrical signals from the ECoG array to a detachable digital headstage and HDMI cable (CerePlex E256; Blackrock Microsystems). The digital headstage minimally processes and digitizes the acquired cortical signals and then transmits the data to a computer for further signal processing. The device was implanted in September 2022 at UCSF Medical Center with no surgical complications.

### 1.5.3. Signal Processing

The same signal-processing pipeline was used detailed in previous work [3] to extract high-gamma activity (HGA) and low-frequency signals (LFS) from the ECoG signals at a 200-Hz sampling rate. For data normalization, a 30-second sliding-window z-score was applied in real time to the HGA and LFS features from each ECoG channel.

All data collection and real-time decoding tasks were performed in the common area of the participant's residence. A custom Python package named rtNSR was created and used, to collect and process all data, run the tasks, and coordinate the real-time decoding processes. After each session, the neural data was uploaded to the lab's server infrastructure, where the data and trained decoding models were analyzed.

### 1.5.4. Task Design

Experimental paradigms: To collect training data for the decoding models, a task paradigm was implemented in which the participant attempted to produce prompted targets. In each trial of this paradigm, the participant was presented with text representing a speech target (for example, “Where was he trying to go?”) or a non-speech target (for example, “Lips back”). The text was surrounded by three dots on both sides, which sequentially disappeared to act as a countdown. After the final dot disappeared, the text turned green to indicate the go cue, and the participant attempted to silently say that target or perform the corresponding action. After a brief delay, the screen cleared and the task continued to the next trial.

During real-time testing, three different task conditions were used: text, audio-visual, and NATO-motor. The text task condition was used to evaluate the text decoder. In this condition, the top half of the screen was used to present prompted targets to the participant, similar to what was used for training. The bottom half of the screen was used to display an indicator (three dots) when the text decoder first predicted a non-silence phone, which was updated to the full decoded text once the sentence was finalized.

The audio-visual task condition was used to evaluate the speech-synthesis and avatar-animation models, including the articulatory-movement and emotional-expression classifiers. In this condition, the participant attended to a screen showing the Unreal Engine environment that contained the avatar. The viewing angle of the environment was focused on the avatar's face. In each trial, speech and non-speech targets appeared on the screen as white text. After a brief delay, the text turned green to indicate the go cue, and the participant attempted to silently say that target or perform the corresponding action. Once the decoding models processed the neural data associated with the trial, the decoded predictions were used to animate the avatar and, if the current trial presented a speech target, play the synthesized speech audio.

The NATO-motor task condition was used to evaluate the NATO code-word classification model and to collect neural data during attempted hand-motor movements. This task contained 26 speech targets (the code words in the NATO phonetic alphabet) and 4 non-speech hand-motor targets (left-thumb flexion, right-thumb flexion, right index- and middle-finger flexion, and left index- and middle-finger flexion). This task condition resembled the text condition, except that the top three predictions from the classifier (and their corresponding predicted probabilities) were shown in the bottom half of the screen as a simple horizontal bar chart after each trial. The prompted-target paradigm was used to collect the first few blocks of this dataset, and then switched to the NATO-motor task condition to collect all subsequent data and to perform real-time evaluation.

Sentence sets: Three different sentence sets were used in this work: “50-phrase-AAC”, “529-phrase-AAC”, and “1024-word-General.” The first two sets contained sentences that are relevant for general dialogue as well as augmentative and alternative communication (AAC) [4]. The 50-phrase-AAC set contained 50 sentences composed of 119 unique words, and the 529-phrase-AAC set contained 529 sentences composed of 372 unique words and included all of the sentences in the 50-phrase-AAC set. The 1024-word-General set contained sentences sampled from Twitter and movie transcriptions for a total of 13,463 sentences and 1,024 unique words (Method 1).

To create the 1024-word-General sentence set, sentences were first extracted from the nltk Twitter corpus and the Cornell movies corpus [54]. All sentences were selected from this corpora that were composed entirely from the 1,152-word vocabulary from previous work [3], which contained common English words. This resulted in a set of 18,284 total sentences. Offensive sentences, sentences that grammatically did not make sense, and sentences with overly negative connotation were then subjectively pruned out, which resulted in 13,463 sentences composed of a total of 1,024 unique words. Partway through training, sentences with syntactic pauses or punctuation in the middle were removed (Method 1). Of these sentences, the participant was able to go through 9,512 of them for use as training data.

250 sentences were randomly selected from the 1024-word-General set to use as the final test sentences for text decoding. Training data was did not collect with these sentences as targets. For evaluation of audio-visual synthesis and avatar, 200 sentences were randomly selected that were not used during training and were not included in the 250 sentences used for text-decoding evaluation (Method 1). As a result of the prior reordering, the audio-visual synthesis and avatar test sets contained a larger proportion of common words.

For training and testing with the 1024-word-General sentence set, to help the decoding models infer word boundaries from the neural data without forgoing too much speed and naturalness, the participant was instructed to insert small syllable-length pauses (approximately 300-500 ms) between words during her silent speech attempts. For all other speech targets, the participant was instructed to attempt to silently speak at her natural rate.

### 1.5.5. Text Decoding

Phone decoding: For the text-decoding models, the neural signals were downsampled by a factor of 6 (from 200 Hz to 33.33 Hz) after applying an anti-aliasing low-pass filter at 16.67 Hz using the Scipy python package [55]. The high-gamma activity and low-frequency signals were then normalized separately to have an L2-norm of 1 across all time steps for each channel. All available electrodes were used during decoding.

A recurrent neural network (RNN) was trained to model the probability of each phone at each time step, given these neural features. The RNN was trained using the connectionist temporal classification (CTC) loss [5] to account for the lack of temporal alignment between neural activity and phone labels. The CTC loss maximizes the probability of any correct sequence of phone outputs that correspond to the phone transcript of a given sentence. To account for differences in the length of individual phones, the CTC loss collapses over consecutive repeats of the same phone. For example, predictions corresponding to /w [symbol] z/—the phonetic transcription of “was”—could be a result of the RNN predicting the following valid time series of phones: /w [symbol] z z/,/w w [symbol] [symbol] z/z/, /w w [symbol] z/, and so forth.

Reference sequences were determined using g2p-en [56], a grapheme-to-phoneme model that enabled recovery of phone pronunciations for each word in the sentence sets. A silence token was inserted in between each word and at the beginning and end of each sentence. For simplicity, a single phonetic pronunciation was used for each word in the vocabulary. These sentence-level phone transcriptions were used for training and to measure performance during evaluation.

The RNN itself contained a convolutional portion followed by a recurrent portion. The convolutional portion of the RNN was composed of a 1-D convolutional layer with 500 kernels, a kernel size of 4, and a stride of 4. The recurrent portion was composed of 4 layers of bidirectional gated recurrent units with 500 hidden units. The hidden states of the final recurrent layer were passed through a linear layer and projected into a 41-dimensional space. These values were then passed through a softmax activation function to estimate the probability of each of the 39 phones, the silence token, and the CTC blank token (used in the CTC loss to predict two tokens in a row or to account for silence at each time step [5]). These models were implemented using the PyTorch Python package (version 1.10.0) [59].

The RNN was trained to predict phone sequences using an 8-second window of neural activity. To improve the model's robustness to temporal variabilities of the patient's productions, jitter was introduced during training by randomly sampling a continuous 8-second window from a 9-second window of neural activity spanning from 1 second before to 8 seconds after the go cue, as in previous work [2] [3]. During inference, the model used a window of neural activity beginning 500 ms prior to the go cue. To improve communication rates and decoding of variable-length sentences, trials were terminated before a full 8-second window if the decoder determined the participant had stopped attempted speech by using silence detection. To implement this early-stopping mechanism, the following steps were performed: 1) Starting 1.9 s after the go cue and then every 800 ms afterwards, the RNN was used to decode the neural features acquired up to that point in the trial; 2) If the RNN predicted the silence token for the most recent 8 time steps (960 ms) with over 88.8% average probability (or, in 2 out of the 250 real-time test trials, if the 7.5-second trial duration expired), the current sentence prediction was used as the final model output and the trial ended. A version of the task was attempted where the current decoded text was presented to the participant every 800 ms; however, the participant preferred only seeing the finalized decoded text. See Method 2 for further details about the data-processing, data-augmentation, and training procedures used to fit the RNN and Table 12 for hyperparameter values.

TABLE 12
NATO code word and hand-motor movement
decoder hyperparameters.

| | | Final |
|---|---|---|
| Sentence set | Hyperparameter description | Value |
| 529-phrase-AAC | Kernel size (and stride) | 4 |
| sentence set | Number of GRU layers | 3 |
| | Hidden units per GRU layer | 500 |
| | Dropout | 0.4 |
| | Jitter amount j | 0.5 |
| | Additive noise level σ [symbol] | 0.0027 |
| | Scale [symbol] | 0.955 |
| | Scale [symbol] | 1.07 |
| | Max temporal mask length b | 0.871 |
| | Temporal mask probability p | 0.0478 |
| | Channel-wise noise [symbol] | 0.0283 |
| 50-phrase-AAC | Kernel size (and stride) | 2 |
| and 529-phrase-AAC | Number of GRU layers | 3 |
| sentence set | Hidden units per GRU layer | [symbol] 12 |
| | Dropout | [symbol] .6 |

[symbol]

indicates data missing or illegible when filed

Beam-search algorithm: A CTC beam-search algorithm was used to transform the predicted phone probabilities into text [60]. To implement this CTC beam search, the ctc_decode function was used in the torchaudio Python package [61]. Briefly, the beam search finds the most likely sentence given the phone probabilities emitted by the RNN. For each silent speech attempt, the likelihood of a sentence is computed as the emission probabilities of the phones in the sentence combined with the probability of the sentence under a language-model prior. A custom-trained 5-gram language model [62] was used with Kneser-ney smoothing [63]. The KenLM software package [64] was used to train the 5-gram language model on the full 18,284 sentences that were eligible to be in the 1024-word-General set prior to any pruning. This approach was chosen because the linguistic structure and content of conversational tweets and movie lines are more relevant for everyday usage than formal written language commonly used in many standard speech-recognition databases [65] [66]. The beam search also uses a lexicon to restrict phone sequences to form valid words within a limited vocabulary. Here, a lexicon defined by passing each word in the vocabulary through a grapheme-to-phoneme conversion module (g2p-en) was used to define a valid pronunciation for each word. A language model weight of 4.5 was used and a word insertion score of −0.26 (Method 2).

Decoding Speed: To measure decoding speed during real-time testing, the formula N/T was used, where N is the number of words in the decoded output and T is the time (in minutes) that the participant was attempting to speak. A lower-bound estimate of T was used by computing the elapsed time between the appearance of the go cue and the time of the data sample which immediately preceded the samples that triggered early stopping.

Error-rate calculation: Word error rate (WER) is defined as the word edit distance, which is the minimum number of word deletions, insertions, and substitutions required to convert the decoded sentence into the target (prompted) sentence, divided by the number of words in the target sentence. Phone error rate (PER) and character error rate (CER) are defined analogously for phones and characters, respectively. When measuring PERs, the silence token at the start of each sentence were ignored, since this token is always present at the start of both the reference phone sequence and the phone decoder's output.

Offline simulation of large-vocabulary, 50-phrase-AAC, and 500-phrase-AAC results: To simulate text-decoding results using the 42,391-word vocabulary, the same neural activity, RNN decoder, and start and end times were used that were used during real-time evaluation. The underlying language model was only changed to be a 5-gram language model that was trained on the entirety of the nltk Twitter and Cornell movies corpora. Then, a readily available pronunciation dictionary from the Librispeech Corpus [65] was used, and all words were selected which were present in the Twitter and Cornell movies corpora (for a total of 42,391 words). The results were then simulated on the task with the larger vocabulary and language model.

To simulate text-decoding results on the 50-phrase-AAC and 500-phrase-AAC sentence sets (because the text decoder was only tested in real time with the 1024-word-General set), RNN decoders were trained on data associated with these two AAC sets (Method 2; see Table 12 for hyperparameter values). Decoding was then simulated using the neural data and go cues from the real-time blocks used for evaluation of the avatar and synthesis methods. Early stopping was checked for 2.2 s after the start of the sentence and again every subsequent 350 ms. Once an early stop was detected, or if 5.5 seconds had elapsed since the go cue, the sentence prediction was finalized. During decoding, the CTC beam search was applied using a 5-gram language model fit on the phrases from that set.

Decoding NATO code words and hand-motor movements: The same neural-network decoder architecture was used (but with a modified input and output layer dimensionality to account for differences in the number of electrodes and target classes) as in previous experiments [3] to output the probability of each of the 26 NATO code words and the four hand-motor targets. To maximize data efficiency, transfer learning was used between the participants; the decoder was initialized using weights from previous performed experiments, and the first and last layers to were replaced account for differences in the number of electrodes and number of classes being predicted, respectively. See Method 3 for further details about the data-processing, data-augmentation, and training procedures used to fit the classifier and Table 13 for hyperparameter values. For the results shown in FIG. 4B, NATO code-word classification accuracy was computed using a model that was also capable of predicting the motor targets; here, performance was only measured on trials in which the target was a NATO code word, and any such trial in which a code-word attempt was misclassified as a hand-motor attempt was deemed incorrect.

TABLE 13
NATO code word and hand-motor movement
decoder hyperparameters.

| Hyperparameter description | Final Value |
|---|---|
| Kernel size (and stride) | 4 |
| Number of GRU layers | 2 |
| Hidden units per GRU layer | 274 |
| Dropout | 0.545 |
| Jitter amount j | .237 |
| Additive noise level σₙ | 0.0027 |
| Scale min αₘᵢₙ | 0.955 |
| Scale max αₘₐₓ | 1.07 |
| Max temporal mask length b | 0.871 |
| Temporal mask probability p | 0.0478 |
| Channel-wise noise σ [symbol] | 0.0283 |

[symbol]

indicates data missing or illegible when filed

### 1.5.6. Speech Synthesis

Training and inference procedure: CTC loss was used to train an RNN to predict a temporal sequence of discrete speech units extracted using HuBERT [13] from neural data. HuBERT is a speech-representation learning model that is trained to predict acoustic k-means-cluster identities corresponding to masked timepoints from unlabeled input waveforms. These cluster identities were referred to as discrete speech units, and the temporal sequence of these speech units represents the content of the original waveform.

Because the participant cannot speak, reference sequences of speech units were generated by applying HuBERT to a speech waveform which was referred to as the basis waveform. For the 50-phrase-AAC and 529-phrase-AAC sets, basis waveforms were acquired from a single male speaker (recruited prior to the participant's enrollment in the trial) who was instructed to read each sentence aloud in a consistent manner. Due to the large number of sentences in the 1024-word-General set, the Wavenet text-to-speech model [67] was used to generate basis waveforms.

HuBERT was used to process the basis waveforms and generate a series of reference discrete speech units sampled at 50 Hz. The base 100-unit, 12-transformer-layer HuBERT trained on 960 hours of LibriSpeech [65] was used, which is available in the open-source fairseq library [68]. In addition to the reference discrete speech units, the blank token needed for CTC decoding was added as a target during training.

The synthesis RNN, which was trained to predict discrete speech units from the ECoG features (high-gamma activity and low-frequency signals), consisted of the following layers (in order): 1) A 1-D convolutional layer, with 260 kernels with width and stride of 6; 2) 3 layers of bidirectional gated recurrent units, each with a hidden dimension size of 260; and 3) a 1-D transpose convolutional layer, with a size and stride of 6, that output discrete-unit logits. To improve robustness, data augmentations were applied using the SpecAugment method to the ECoG features during training. See Method 4 for the complete training procedure and Table 14 for hyperparameter values.

TABLE 14
Speech synthesis RNN neural-decoding model hyperparameters.

| | | Final |
|---|---|---|
| Sentence set | Hyperparameter description | Value |
| 1024-word-General | Kernel size (and stride) | 6 |
| | Number of GRU layers | 3 |
| | Hidden units per GRU layer | 260 |
| | Dropout | 0.7 |
| | Batch size | 1 [symbol] |
| 529-phrase-AAC | Kernel size (and stride) | 6 |
| and | Number of GRU layers | 3 |
| 50-phrase-AAC | Hidden units per GRU layer | 260 |
| | Dropout | 0.7 |
| | Batch size | 64 |

[symbol]

indicates data missing or illegible when filed

From the ECoG features, the RNN predicted the probability of each discrete unit every 5 ms. Only the most likely predicted unit was retained at each time step. Time steps where the CTC blank token was decoded were ignored, as this is primarily used to adjust for alignment and repeated decodes of discrete units. Next, a speech waveform was synthesized from the sequence of discrete speech units, using a pre-trained unit-to-speech vocoder [70].

During each real-time inference trial in the audio-visual task condition, the speech-synthesis model was provided with ECoG features collected in a time window around the go cue. This time window spanned from 0.5 seconds before to 4.62 seconds after the go cue for the 50-phrase-AAC the 529-phrase-AAC sentence sets and from 0 seconds before to 7.5 seconds after the go cue for the 1024-word-General sentence set. The model then predicted the most likely sequence of HuBERT units from the neural activity and generated the waveform using the aforementioned vocoder. The waveform was streamed in 5-ms chunks of audio directly to the real-time computer's sound card via the PyAudio Python package.

To decode speech waveforms in the participant's personalized voice (that is, a voice designed to resemble the participant's own voice before her injury), YourTTS [71] was used, a zero-shot voice conversion model. After conditioning the model on a short clip of the participant's voice extracted from a pre-injury video of her, the model was applied to the decoded waveforms to generate the personalized waveforms.

Evaluation: To evaluate the quality of the decoded speech, the mel-cepstral distortion (MCD) was computed between the decoded and reference waveforms (ŷ and y, respectively) [17]. This is defined as the squared error between dynamically time warped sequences of mel cepstra extracted from the target and decoded waveforms and is commonly used to evaluate the quality of synthesized speech:

$$M C D ( \hat{y ,} y ) = \frac{10}{ln ( 10 )} \sqrt{{\sum }_{d = 1}^{24} {( m {c}_{d}^{y} + m {c}_{d}^{\hat{y}} )}^{2}}$$

Silence time points were excluded at the start and end of each waveform during MCD calculation.

A perceptual assessment was designed using a crowd-sourcing platform (Amazon Mechanical Turk), where each test trial was assessed by 12 evaluators (except for 3 of the 500 trials, in which only 11 workers completed their evaluations). In each evaluation, the evaluator listened to the decoded speech waveform and then transcribed what they heard (Method 4). For each sentence, the WER and CER were then computed between the evaluator's transcriptions and the ground-truth transcriptions. To control for outlier evaluator performance, for each trial, the median WER and CER was used across evaluators as the final accuracy metric for the decoded waveform. Metrics across pseudo-blocks of 10 sentences were reported to be consistent with text-decoding evaluations.

### 1.5.7. Avatar

Articulatory-gesture data: A dataset of articulatory gestures was used for all sentences from the 50-phrase-AAC, 529-phrase-AAC, and 1024-word-general datasets provided by Speech Graphics. These articulatory gestures were generated from reference waveforms using Speech Graphics' speech-to-gesture model, which was designed to animate avatar movements given a speech waveform. For each trial, articulatory gestures consisted of 16 individual gesture time series corresponding to jaw, lip, and tongue movements (Table 5).

Offline training and inference procedure for the direct avatar-animation approach: To perform direct decoding of articulatory gestures from neural activity (the direct approach for avatar animation), a vector-quantized variational autoencoder (VQ-VAE) was first trained to encode continuous Speech Graphics' gestures into discrete articulatory-gesture units [29]. A VQ-VAE is composed of an encoder network that maps a continuous feature space to a learned discrete codebook and a decoder network that reconstructs the input using the encoded sequence of discrete units. The encoder was composed of 3 layers of 1-D convolutional units with 40 filters, a kernel size 4, and a stride of 2. Rectified linear unit (ReLU) activations followed the second and third of these layers. After this step, a 1-D convolution was applied, with 1 filter and a kernel size and stride of 1, to generate the predicted codebook embedding. Nearest-neighbor lookup was then used to predict the discrete articulatory-gesture units. A codebook with 40 different 1-D vectors was used, wherein the index of the codebook entry with the smallest distance to the encoder's output served as the discretized unit for that entry. The VQ-VAE's decoder was trained to convert discrete sequences of units back to continuous articulatory gestures by associating each unit with the value of the corresponding continuous 1-D codebook vector. Next, a 1-D convolution layer was applied, with 40 filters and a kernel size and stride of 1, to increase the dimensionality. Then, 3 layers of 1-D transpose convolutions were applied, with 40 filters, a kernel size of 4, and a stride of 2, to upsample the reconstructed articulatory gestures back to their original length and sampling rate. ReLU activations followed the first and second of these layers. The final 1-D transpose convolution had the same number of kernels as the input signal (16). The output of the final layer was used as the reconstructed input signal during training.

To encourage the VQ-VAE units to decode the most critical gestures (such as jaw opening) rather than focusing on those that are less important (such as nostril flare), the mean-squared error (MSE) loss was weighted more highly for the most important gestures. The jaw opening's MSE loss was upweighted by a factor of 20, and the gestures associated with important tongue movements (tongue body raise, tongue advance, tongue retraction, tongue tip raise) and lip movements (rounding and retraction) by a factor of 5. The VQ-VAE was trained using all of the reference articulatory-gestures from the 50-phrase-AAC, 529-phrase-AAC, and 1024-word-General sentence sets. VQ-VAE was excluded from training any sentence that was used during the evaluations with the 1024-word-General set.

To create the CTC decoder, a bidirectional RNN was trained to predict reference discretized articulatory-gesture units given neural activity. The ECoG features were first downsampled by a factor of 6 to 33.33 Hz. These features were then normalized to have an L2-norm of 1 at each time point across all channels. A time window of neural activity spanning from 0.5 s before to 7.5 seconds after the go cue was used for the 1024-word-General set and from 0.5 s before to 5.5 seconds after for the 50-phrase-AAC and 529-phrase-AAC sets. The RNN then processed these neural features using the following components: 1) A 1-D convolution layer, with 256 filters with a kernel size and stride of 2; 2) 3 layers of gated recurrent units, each with a hidden dimension size of 512; and 3) A dense layer, which produced a 41-dimensional output. The softmax activation function was then used to output the probability of the 40 possible discrete units (determined by the VQ-VAE) as well as the CTC blank token. See Method 5 for full training details for the VQ-VAE and CTC decoder. The model hyperparameters stated here are for the 1024-word-General sentence set; see Table 15 for other hyperparameter values.

TABLE 15
Avatar CTC decoding hyperparameters.

| | | Final |
|---|---|---|
| Sentence set | Hyperparameter description | Value |
| 1024-word-General | Kernel size (and stride) | 4 |
| | Number of GRU layers | 3 |
| | Hidden units per GRU layer | 500 |
| | Dropout | 0.4 |
| 529-phrase-AAC | Kernel size (and stride) | 2 |
| | Number of GRU layers | 8 |
| | Hidden units per GRU layer | 256 |
| | Dropout | 0.7 |
| 50-phrase-AAC | Kernel size (and stride) | 2 |
| | Number of GRU layers | 3 |
| | Hidden units per GRU layer | 256 |
| | Dropout | 0.7 |
| All sets | Additive noise level [symbol] | 0.0027 |
| | Scale min αₘᵢₙ | 0.955 |
| | Scale max αₘₐₓ | 1.07 |
| | Max temporal mask length b | 0.871 |
| | Temporal mask probability p | 0.0478 |
| | Channel-wise noise [symbol] | 0.0283 |
| | Weight decay | 1e−5 |

[symbol]

indicates data missing or illegible when filed

During inference, the RNN yielded a predicted probability of each discretized articulatory-gesture unit at every 60 ms. To transform these output probabilities into a sequence of discretized units, only the most probable unit at each time step was retained. The decoder module of the frozen VQ-VAE was used to transform collapsed sequences of predicted discrete articulatory units (here, “collapsed” means that consecutive repeats of the same unit were removed) into continuous articulatory gestures.

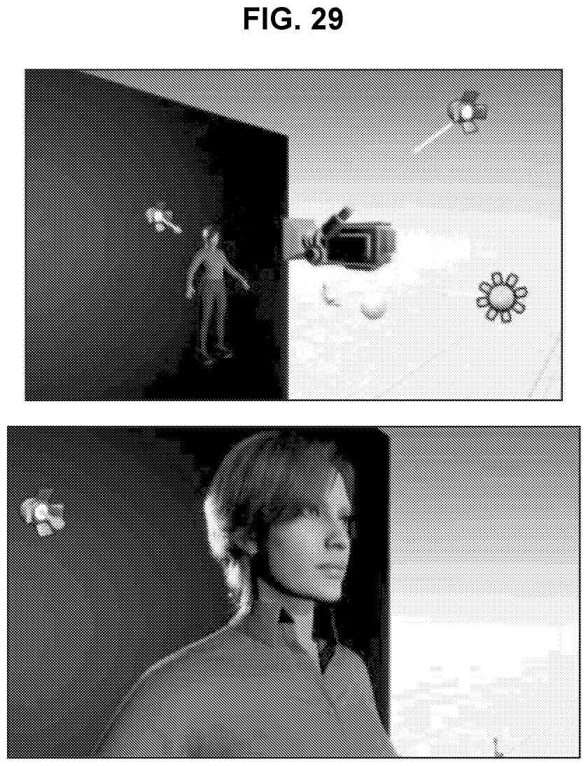

Real-time acoustic avatar-animation approach: During real-time testing, the avatar was animated using avatar-rendering software (referred to as SG Com; provided by Speech Graphics; FIG. 29). This software converts a stream of speech audio into synchronized facial animation with a latency of 50 ms. It performs this conversion in two steps: First, it uses a custom speech-to-gesture model to map speech audio to a time series of articulatory-gesture activations; Then, it performs a forward mapping from articulatory-gesture activations to animation parameters on a 3-D MetaHuman character created by Epic Games, Inc (Cary, North Carolina). The output animation was rendered using Unreal Engine 4.26 [65] (Method 5).

For every 10 ms of input audio, the speech-to-gesture model produces a vector of articulatory-gesture activation values, each between 0 and 1 (where 0 is fully relaxed and 1 is fully contracted). The forward mapping converts these activations into deformations, simulating the effects of the articulatory gestures on the avatar face. Because each articulatory gesture approximates the superficial effect of some atomic action, such as opening the jaw or pursing the lips, the gestures are analogous to the Action Units of the Facial Action Coding System [73], a well known method for taxonomizing human facial movements. However, these articulatory gestures from Speech Graphics are more oriented toward speech articulation and also include tongue movements, containing 16 speech-related articulatory gestures (10 for lips, 4 for tongue, 1 for jaw, and 1 for nostril). The system does not generate values for aspects of the vocal tract that are not externally visible, such as the velum, pharynx, or larynx.

To provide avatar feedback to the participant during real-time testing in the audio-visual task condition, 10-ms chunks of decoded audio were streamed over an Ethernet cable to a separate machine running the avatar processes to animate the avatar in synchrony with audio synthesis. A 200-ms delay was imposed on the audio output in real time to improve perceived synchronization with the avatar.

The avatar-rendering system also generates non-verbal motion, such as emotional expressions, head motion, eye blinks, and eye darts. These are synthesized using a superset of the articulatory gestures involving the entire face and head. These non-verbal motions are used during the audio-visual task condition and emotional-expression real-time decoding.

FIG. 29 illustrates a virtual environment for avatar decoding. Virtual environment (designed in Unreal Engine 4.26) containing the camera and setup (left) for the “Vivian” MetaHuman's character (right).

Speech-related animation evaluation: To evaluate the perceptual accuracy of the decoded avatar animations, a crowd-sourcing platform (Amazon Mechanical Turk) was used to design and conduct a perceptual assessment of the animations. Each decoded animation was assessed by 6 unique evaluators. Each evaluation consisted of playback of the decoded animation (with no audio) and textual presentation of the target (ground-truth) sentence and a randomly chosen other sentence from the same sentence set. Evaluators were instructed to identify the phrase that they thought the avatar was trying to say (Method 5). The accuracy was computed for median accuracy of the evaluations across evaluators for each sentence and that was treated as the accuracy for a given trial and then the final accuracy distribution was computed using the pseudo-block strategy described above.

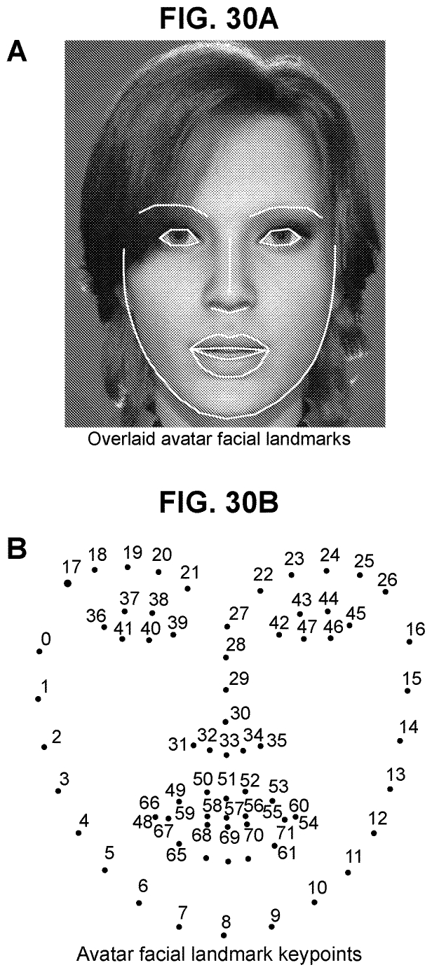

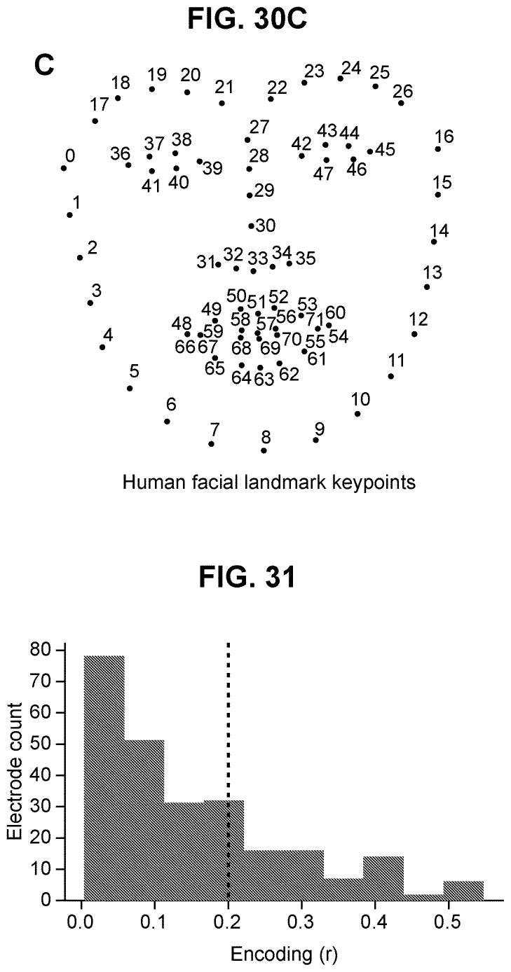

Separately, the dlib software package was used to extract 72 facial keypoints for each frame in avatar-rendered and healthy-speaker videos (sampled at 30 frames per second). To obtain videos of healthy speakers, video and audio of 8 volunteers were recorded as they produced the same sentences used during real-time testing in the audio-visual task condition. The keypoint positions were normalized relative to other keypoints to account for head movements and rotation: jaw movement was computed as the distance between the keypoint at the bottom of the jaw and the nose, lip aperture as the distance between the keypoints at the top and bottom of the lips, and mouth width as the distance between the keypoints at either corner of the mouth (Method 5; FIG. 30). To compare avatar keypoint movements to those for healthy speakers, and to compare amongst healthy speakers, dynamic time warping was first applied to the movement time series and then the Pearson's correlation between the pair of warped time series was computed. 10 of 200 1024-word-General avatar videos were held out from final evaluation since they were used to select parameters to automatically trim the dlib traces to speech onset and offset. This was done because the automatic segmentation method relied on the acoustic onset and offset, which is absent from direct avatar decoding videos.

FIGS. 30A to 30C provide examples of dlib facial-landmark detection. (A) an example of detected facial landmarks overlaid on a frame selected from a video rendering of an avatar during a single trial and using the direct approach to avatar decoding. (B) an example set of plotted detected facial landmark key points from a video rendering of an avatar during a single trial and using the direct approach to avatar decoding. (C) an example set of plotted facial landmark key points that shows exemplar facial landmark detection of a human face. The original video frame was selected from one of 8 volunteer speakers speaking a phrase from the 1024-word-General sentence set. The avatar and human key points are coherently and robustly tracked. All landmarks were tracked and detected using [30]. In (B) and (C), the small red arrow points to the precise xy coordinate of the landmark tracked by each labeled keypoint. Note some landmarks are overlapping (e.g., keypoints 48 and 66).

Articulatory-movement decoding: To collect training data for non-verbal orofacial-movement decoding, the articulatory-movement task was used. Prior to data collection, the participant viewed a video of an avatar performing the following 6 movements: open mouth, pucker lips, lips back (smiling or lip retraction), raise tongue, lower tongue, and close mouth (rest or idle). Then, the participant performed the prompted-target task containing these movements as targets (presented as text).

To train and test the avatar-movement classifier (Method 5), a window of neural activity spanning from 1 second before to 3 seconds after to the go cue was used for each trial. The ECoG features (high-gamma activity and low-frequency signals) were first downsampled by a factor of 6 to 33.33 Hz. These features were then normalized to have an L2-norm of 1 at each time point across all channels separately for the low-frequency and high-gamma signals. Next, the mean, minimum, maximum, and standard deviation were extracted across the first and second halves of the neural time window for each feature. These features were then stacked to form a 4048-dimensional neural-feature vector (the product of 256 electrodes, 2 feature sets, 4 statistics, and 2 data halves) for each trial. A multi-layer perceptron was then trained consisting of 2 linear layers with 512 hidden units and ReLU activations between the first and second layer. The final layer projected the output into a 6-dimensional output vector. A softmax activation was then applied to get a probability of each of the 6 different gestures. The network was evaluated using 10-fold cross-validation.

Emotional-expression decoding: To collect training data for non-verbal emotional-expression decoding, the emotional-expression task was used. Using the prompted-target task paradigm, neural data was collected as the participant attempted to produce three emotions (sad, happy, and surprised) at three intensity levels (high, medium, and low) for a total of 9 unique expressions. The same data-windowing and neural-processing steps as for the articulatory-movement decoding was used. The same model architecture and training procedure was used as for the NATO-and-hand-motor classifier. The expression classifier was initialized with a pre-trained NATO-and-hand-motor classifier (trained on 1,222 trials of NATO-motor task data collected prior to the start of collection for the emotional-expression task) and fine-tuned the weights on neural data from the emotional-expression task. See Method 5 for further details on the data augmentation, ensembling, and hyperparameter values used with this model.

The expression classifier was evaluated using 15-fold cross validation. Within the training set of each cross-validation fold. For each fold, 10 unique models were fit to ensemble predictions on the held-out test set. Hierarchical agglomerative clustering was applied to the 9-way confusion matrix in FIG. 10B using SciPy [55].

### 1.5.8. Articulatory-Encoding Assessments

To investigate the neural representations driving speech decoding, the selectivity of each electrode to articulatory groups of phones was assessed. Specifically, a linear receptive-field encoding model was fit to predict each electrode's high gamma activity (HGA) from phone-emission probabilities predicted by the text-decoding model during 10-fold cross-validation with data recorded with the 1024-word-General sentence set. The HGA was first decimated by a factor of 24, from 200 Hz to 8.33 Hz, to match the sampling rate of the phone-emission probabilities. Then, a linear receptive-field model was fit to predict the HGA at each electrode, using the phone-emission probabilities as time-lagged input features (39 phones and 1 aggregate token representing both the silence and CTC-blank tokens). A +/−4-sample (480-ms) receptive-field window was used, allowing for slight misalignment between the text decoder's bidirectional-RNN phone-emission probabilities and the underlying HGA. An independent model was fit for each electrode. The true HGA, HGA (t), is modeled as a weighted linear combination of phone-emission probabilities (indexed by p) in the overall emissions matrix (X) over a +/−4-sample window around each time point. This resulted in a learned weight matrix w(d,p) in which each phone, p, has temporal coefficients d₁ . . . . D, where d₁ is −4 and D is 4. During training the squared error between the predicted HGA, HGA*(t), and the true HGA, HGA(t), is minimized, Using the following formulas:

$$H G {A}^{*} ( t ) = {\sum }_{d = 1}^{D} {\sum }_{p = 1}^{P} w ( d , p ) * X ( p , t - d ) ,$$

$$min {{\sum }_{t} [ H G {A}^{*} ( t ) - H GA ( t ) ]}^{2} .$$

The model was implemented with the MNE toolbox's receptive-field ridge regression in Python [74]. Cross-validation was used to select the alpha ridge-regression parameter by sweeping over the values [1e-1, 1e0, 1e1, . . . 1e5], using 10% of the total data as a held-out tuning set. The remaining 90% of the total data was used for 10-fold cross-validation with the alpha parameter found optimal on the tuning set. The coefficients were averaged for the model across the 10 folds and collapsed across time samples for every phone using the maximum magnitude weight. The sign of the weight could be positive or negative. This yielded a single vector for each electrode, where each element in each vector was the maximum encoding of a given phone. Next, any electrode channels that were not significantly modulated by silent speech attempts were pruned. For each electrode, the mean HGA magnitudes were computed in the 1-s intervals immediately before and after the go cue for each NATO code-word trial in the NATO-motor task. If an electrode did not have significantly increased HGA after the go cue compared to before, it was excluded from the remainder of this analysis (significant modulation determined using one-sided Wilcoxon signed-rank tests with an alpha level of 0.001 after applying 253-way Holm-Bonferroni correction). A second pruning step was then applied to exclude any electrodes that had encoding values (r) less than or equal to 0.2 (FIG. 31). The centroid clustering method, a hierarchical, agglomerative clustering technique, was applied to the encoding vectors using the SciPy Python package [75]. Clustering was performed along both the electrode and phone dimensions.

To assess any relationships between phone encodings and articulatory features, each phone was assigned to a place-of-articulation (POA) feature category [24] [25]. Specifically, each phone was either primarily articulated at the lips (labial), the front tongue, the back tongue, or larynx (vocalic). To quantify whether the unsupervised phone-encoding clusters reflected grouping by POA, the null hypothesis that the observed parcellation of phones into clusters was not more organized by POA category than by chance was tested. To test this null hypothesis, the following steps were used: 1) Compute the POA linkage distances by clustering the phones by Euclidean distance into F clusters, where F=4 is the number of POA categories; 2) Randomly shuffle the mapping between the phone labels and the phonetic encodings; 3) For each POA category, compute the maximum number of phones within that category that appear within a single cluster; 4) Repeat steps 2 and 3 over a total of 10,000 bootstrap runs; 5) Compute the pairwise Euclidean distance between all combinations of the 10,000 bootstrap results; 6) Repeat step 3 using the true unsupervised phone ordering and clustering; 7) Compute the pairwise Euclidean distances between the result from step 6 and each bootstrap from step 4; 8) Compute the one-tailed Wilcoxon rank-sum test between the results from step 7 and step 5. The resulting P-value is the probability of the aforementioned null hypothesis.

To visualize population-level (across all electrodes that were not pruned from the analysis) encoding of POA features, the mean encoding of each electrode was first computed across the 4 POA feature groups (vocalic, front tongue, labial, and back tongue). The mean encodings for each POA feature were then z-scored and multidimensional scaling MDS then applied over the electrodes to visualize each phone in a 2-dimensional space. This was implemented using the scikit-learn Python package [76].

To measure somatotopy, kernel density estimations of the locations of top electrodes (the 30% of electrodes with the strongest encoding weights) were computed for each POA category along anterior-posterior and dorsal-ventral axes (FIG. 4A). To do this, the seaborn Python package [77], Gaussian kernels, and Scott's Rule were used.

To quantify the magnitude of activation in response to non-verbal orofacial movements, the median of the evoked response potential to each action over the time window spanning from 1 s before to 2 s after the go cue was taken. From this, the same metric computed across all actions was subtracted to account for electrodes that were non-differentially task-activated. For each action, values were then normalized across electrodes to be between 0 and 1. Ordinary least squares linear regression was used, implemented by the statsmodels Python package [78], to relate phone-encoding weights with activation to attempted motor movements.

FIG. 31 depicts the distribution of phone-encoding r-values across electrodes. Shown are encoding r-values across electrodes from the linear encoding model trained to predict each electrode's high gamma activity from phoneme emission probabilities in accordance with an embodiment of the invention. The dashed black line indicates the cut-off for inclusion in subsequent clustering analysis (FIG. 11B). 27.7% of electrodes met a threshold of r>0.2 for inclusion [87].

### 1.5.9. Exclusion Analyses

Each electrode was assigned to an anatomical region and all electrodes were visualized on the pial surface [79]. For the exclusion analyses, the phone-based text-decoding model was tested on the real-time evaluation trials in the text task condition with the 1024-word-General sentence set. Early stopping was not used for these analyses; the full 8-s time windows of neural activity was used for each trial. Also, the NATO code-word classifier was tested by training and testing on NATO code-word trials recorded during the NATO-motor task. All of the NATO-motor task blocks recorded after freezing the classifier (FIG. 4B), a total of 19 blocks, were used as the test set.

### 1.5.10. Statistics

Statistical analyses: Statistical tests are fully described in the figure captions and text. To summarize, two-sided Mann-Whitney Wilcoxon Rank-sum tests were used to compare unpaired distributions. These tests do not assume normally distributed data. For paired comparisons, two-sided Wilcoxon signed-rank tests were used, which also do not assume normally distributed data. When the underlying neural data was not independent across comparisons, the Holm-Bonferroni correction was used for multiple comparisons. P-values<0.01 were considered statistically significant. 99% confidence intervals were estimated using a bootstrapping approach where the distribution (e.g. trials or pseudo-blocks) of interest was randomly sampled with replacement 2000 times and the desired metric was computed. The confidence interval was then computed on this distribution of the bootstrapped metric.

### 1.5.11. Recorded Videos

Video 1: A demonstration of generalizable, real-time text decoding from brain activity. In the task, the participant attempts to silently say sentences from the 1024-word-General sentence set. Each target sentence appears in the top half of the screen and is followed by a countdown. Once the sentence turns green, she attempts to silently say the sentence. Meanwhile, neural data is streamed to a phonetic text-decoding model. Three dots appear in the bottom half of the screen once speech is detected by the model (when a non-silence phone is predicted). The model uses an early-stopping mechanism to estimate when she has completed her attempt, at which time the sentence is finalized and displayed in the bottom half of the screen (replacing the three dots). These sentences were not attempted by the participant during model training.

Video 2: A demonstration of real-time classification of NATO code words and attempted finger flexions from brain activity. In the task, the participant attempts to silently say NATO code words and perform hand-motor movements as prompted by text targets. Each target appears in the top half of the screen and is followed by a countdown. Once the target turns green, she attempts to silently say the code word or perform the hand-motor movement. A classifier processes neural activity associated with the attempt to predict probabilities across the 30 possible targets. Afterwards, the probabilities for the top 3 classes are shown as a bar chart on the bottom of the screen.

Video 3: A demonstration of real-time speech synthesis and avatar animation from brain activity. In the task, the participant attempts to silently say sentences shown on the screen from the 529-phrase-AAC sentence set. Each target sentence is displayed as white text on the screen. Once the sentence turns green, she attempts to silently say the sentence. Meanwhile, neural data is streamed to a speech-synthesis model. This model decodes a sequence of sound features, which is then used to generate an audible speech waveform and simultaneously animate a personalized virtual avatar face.

Video 4: A demonstration of generalizable, real-time speech synthesis and avatar animation from brain activity. In the task, the participant attempts to silently say sentences shown on the screen from the 1024-word-General sentence set. Each target sentence is displayed as white text on the screen. Once the sentence turns green, she attempts to silently say the sentence. Meanwhile, neural data is streamed to a speech-synthesis model. This model decodes a sequence of sound features, which is then used to generate an audible speech waveform and simultaneously animate a personalized virtual avatar face.

Video 5: An offline demonstration of personalized speech synthesis and avatar animation from brain activity. In this simulation, speech waveforms decoded from the participant's brain activity were processed as she attempted to silently say sentences from the 529-phrase-AAC and 1024-word-General sentence sets using a voice-conversion algorithm. This algorithm was conditioned on audio from a short video clip of her speaking in a pre-injury video. The algorithm transformed the decoded waveforms to be in her personalized voice, which was then used to drive the avatar animation. The avatar animation and the personalized decoded voice were synchronized in this video.

Video 6: A demonstration of real-time classification of attempted articulatory movements from brain activity to drive a facial avatar. In the task, the participant attempts to make 6 non-speech orofacial movements as prompted by text targets. Each target is displayed as white text on the screen. Once the target turns green, she attempts to make the prompted articulatory movement. A classifier processes neural activity associated with the attempt to predict which of the 6 possible articulatory movements she had attempted. Afterwards, this prediction is used to animate the avatar from a set of predefined animations associated with each movement.

Video 7: A demonstration of real-time classification of attempted emotional expressions from brain activity to drive a facial avatar. In the task, the participant attempts to make 3 non-speech expressions as prompted by text targets. Each target is displayed as white text on the screen. Once the target turns green, she attempts to make the prompted expression. A classifier processes neural activity associated with the attempt to predict which of the 3 possible expressions she had attempted. Afterwards, this prediction is used to animate the avatar from a set of predefined animations associated with each expression.

### 1.6. METHOD 1: TEXT CORPORA

Text test set and data organization: To create the test sentences for the text decoder, 250 sentences were randomly selected from the entire corpus. By design, these sentences were not used at any point in training any neural decoders, so at test time, models had to generalize to these unseen sentences. To promote coverage of all words in the 1024-word vocabulary, including infrequent words, the corpus was arranged to have any sentence containing any word with fewer than 6 repetitions in the training data to fall within the first half of the training data, such that all infrequent words had already appeared early within data collection. This helped monitor offline model performance on all words in the vocabulary early on during data collection.

Creation of the test dataset for speech-synthesis and avatar evaluations: For synthesis and avatar models, 200 sentences were randomly sampled that had not been used during training of any model and that were not in the test sentences for the text decoder.

### 1.7. METHOD 2: TEXT DECODING

### 1.7.1. Data Preparation

For decoding, a window of neural activity was used, consisting of the high-gamma and low-frequency signals from all electrodes. The details for extraction of high-gamma and low-frequency signals were detailed extensively in previous work [3]. The l2-norm of each channel were them normalized for the high-gamma and low-frequency signals to be 1 across all time-steps for each channel. The window of neural features was larger than the windows seen by the RNN model. Temporal-jittering was used to pull smaller windows from larger trial-relevant windows. For the 1024-word-General sentence set, a window of neural activity from −1 to 8 seconds was used relative to the go-cue for training, with data augmentation that randomly selected an 8 second window of neural activity that started between 0.75 and 0.25 seconds prior to the go cue to make the RNN model more robust to variability in the timing of the participant's productions relative to the go-cue as in previous work [3,2]. The g2p-en python package [56] was used to extract the phonetic pronunciation for each word in the target sentence. Spaces were replaced with a silence token. To account for silence at the start and end of each sentence, the silence token was prepended and appended at the start and end of the sentence. The code 0 was reserved for the blank token traditionally used with the CTC loss [5] then one-hot encoded all the phonemes and the silence token. During training of the RNN models used in real-time for the 1024-word-General sentence set, 95% of the data was randomly selected to use as training set, and 5% as the development set, to use as a held-out set to estimate model performance on unseen data. This development set was used for hyperparameter optimization as well. The data used for real-time demonstrations were recorded after hyperparameter optimization for all models. For the development set used during training for early stopping and evaluation, the model used a fixed window of neural activity from 0.5 seconds prior to the go-cue to 7.5 seconds after.

### 1.7.2. Modeling

RNN model architecture: The RNN consisted of a 1-dimensional convolutional layer, followed by three layers of bidirectional gated-recurrent units (GRUs), which were followed by a linear readout layer. The convolutional layer processes and downsamples the input signals, and finds combinations of signals that form meaningful representations. The GRUs then further process these representations, incorporating temporal structure across the neural time series. GRUs were used since they have been shown to outperform other recurrent architectures on sequence tasks. To improve accuracy, bidirectional gated-recurrent units were used. Dropout layers were used during training after the convolutional layer, as well as between the GRU layers. A dropout rate was used which was found via manual hyperparameter search (see Table 12). The hidden state of the final gated recurrent unit was then passed through a linear layer followed by a softmax activiation to approximate the probability over the 39 phonemes used, plus the silence phone and the blank token used for CTC decoding [5]. The full set of hyperparameters used for decoding can be found in Table 12.

Connectionist temporal classification (CTC) loss: Given a trial of neural data xi and the corresponding ground truth sequence of phones yi, it is desired to maximize the probability of the ground truth sequence of phones yi, given the neural activity xi, p (yi|xi).

Because alignment between xi and yi is unknown, the connectionist temporal classification (CTC) objective aims to maximize the probability of yi over all valid alignments that produce yi. For example, “was”, with pronunciation [w, aa, z] can be produced from the following valid alignments with four timesteps by collapsing over repeated phones: [w, w, aa, z], [w, aa, aa, z], [w, aa, z, z]. To decode silence and word boundaries, the silence phone Ø was also included as a target during training. To allow for the decoding of silence within words that docs not necessarily result in a word boundary, the flexible blank token e was also introduced as a target, which is commonly used in CTC decoding [5].

The blank token can be ignored in the decoded output, hence e.g., [E, w, aa, zz], [w, ϵ, aa, zz], [w, aa, ϵ, zz], [w, aa, zz, ϵ], are also valid alignments with four timesteps for [w, aa, z]. The blank token can also be used to decode repeated tokens—e.g. if one was decoding sequences of letters instead of sequences of phones, collapsing over repeated letters would make it impossible to decode repeated letters, like the ls in ‘hello’. Hence, decoding the blank token between the two instances of the letter 1, then replacing it with the empty string, would enable the word to be decoded.

In the following, the phone at each timestep is denoted as t in each alignment a is denoted as aₜ. Hence aₜ for the valid alignments for “was” in this case is w in all cases except if the alignment is [ϵ, w, aa, zz]. Thus, the CTC objective for the i-th trial of neural data xi and the corresponding label yi, where yi is the ground truth sequence of phones can be expressed as follows:

$$p ( {y}_{i} \vert {x}_{i} ) = \text{?} {\prod }_{t = 1}^{T} {p}_{t} ( {a}_{t} \vert X )$$

$$\text{?} \text{indicates text missing or illegible when filed}$$

Here A_{xi,yi} represents the set of all valid alignments with length equivalent to the length of xi that could produce yi. During the loss calculation, the RNN is used to estimate the per time-step probability pₜ(aₜ|X). In practice, the RNN model's parameters were optimized to minimize the negative log likelihood over all the samples −Σᵢ log p(yi|xi), which is equivalent to maximizing the probability of the training labels yi given the neural data xi, Πᵢ p(yi|xi). PyTorch's CTCLoss function was used to efficiently calculate this loss.

Data augmentations: For the 1024-word-General sentence set set, in order to improve the RNN model performance on unseen data and make the RNN model robust to variability in the neural signals, as in previous work [3], the following set of data augmentations were used:
Temporal jittering: shift the neural features by a time shift t, so that the for a sample xᵢ, xᵢ(t)=xᵢ(t−τ), where τ~U (−j, j), where j is a hyperparameter, and U is the uniform distribution.
Temporal masking, where some neural timepoints of the neural features were randomly set to 0, yielding xᵢ [t₀: t₁]=(1−δₚ), t₁=t₀+s, s~U (0, b). Here to is a randomly drawn time point within xᵢ and p is the probability of δₚ being one, and subsequently the time points being set to 0. Both b and p are hyperparameters.
Additive noise: adding a matrix of random gaussian noise to the neural features, st xᵢ=xᵢ+N (0, τₙ²). Here σₙ² is a hyperparameter.
Channel-wise noise: Offset each channel by a single value randomly sampled from a guassian for each channel, therefore: xi [:, c]=xi+N (0, σ_{ch}²), here σ_{ch} is a hyperparameter shared across all features.
Scaling augmentation: Scale the magnitude of the neural features, such that xᵢ=axᵢ, with α~U[αₘᵢₙ, αₘₐₓ]. Here αₘᵢₙ and αₘₐₓ are both hyperparameters.

For all hyperparameters, hyperparameters were used that were previously found to be effective [3], save the hyperparameter for j, which was found via manual tuning.

Optimization: To train the RNN model for the 1024-word-General sentence set, the AdamW optimizer was used to perform stochastic batch gradient descent. Briefly, AdamW implements decoupled weight decay for model parameter regularization with the Adam adaptive gradient algorithm. A weight decay value λ=1e-5 was used with a learning rate of 1e-3. The default parameters used were β₁=0.9, β₂=0.999, ε=1e-8. A batch size of 64 was used. The gradient for each batch was clipped to have a total l₂-norm of λ_{grad}=1e-4.

CTC beam search: During inference, the RNN model was evaluated on the word error rate (WER) metric, commonly used to evaluate automatic speech recognition systems and speech and text brain-computer interfaces. This required transforming the RNN outputs, given by pₜ(aₜ|xᵢ), (where aₜ represents the probability of a at time t, where a can be any phone, the silence phone Ø, or the blank token e used in CTC decoding), into text. To do this, the transcription Wi was sought for trial i which maximizes the probability:

p_{RNN}(Wᵢ|xᵢ)pₗₘ(Wᵢ)

Here p_{RNN} (Wᵢ|xᵢ) denotes the probability of possible alignments of phones that result in transcription Wi under the RNN, given the neural data for trial i, xᵢ. pₗₘ(Wᵢ) denotes the probability under a language model prior of the transcription Wᵢ. To account for the fact that language models rate sentences as being less likely as words are added, a word insertion penalty or bonus was included, giving:

p_{RNN}(Wᵢ|xᵢ)pₗₘ(Wᵢ)^{α}|Wᵢ|^{β} (S1)

Here, |W| denotes the number of words in Wi, and α and β are hyperparameters for weighting the language model and number of words. It is found the Wi that optimizes equation S1 via a CTC beam-search algorithm, using torchaudio's CTC decode function [61]. The beam-search works by keeping a set of at most B candidate sentences at each timepoint, where B is a hyperparameter. It then adds each phone (or the silence or blank token) to the candidate sentence, and re-evaluates the resulting candidate sentence after the addition using equation S1. Then, only the B most probable sentences are kept. The algorithm repeats until all timesteps have been processed. For the 1024-word-General sentence set, during RNN model training, models were evaluated using α=4, 8=−0.26 and B=100. An n-gram language model [81] trained with n=5 using kenlm that was trained on all 18,284 sentences generated for the conversational set prior to pruning sentences was used. For final evaluations with the 1024-word-General sentence set a small hyperparameter search was performed to evaluate optimal hyperparameters for the language model prior to decoding, and used α=4.5, β=−0.26, and a beam-width of B=3e3.

Model evaluation and early stopping: For all RNN models, starting with the first epoch, the RNN model's WER was kept track of on a set of held out data every 3 epochs. While loss was evaluated at each epoch, to save time, WER was only evaluated every 3rd epoch. If the WER improved, then the current RNN model's weights were saved. If the WER did not improve for 20 evaluations (corresponding to 20 evaluations over 60 epochs), then training was ended.

Hyperparameter optimization for the 1024-word-General sentence set: Due to limited time, hyperparameters were largely hand tuned as data collection occurred. Hyperparameters were first selected for the RNN neural-decoding model use by evaluating its performance with fixed CTC beam search hyperparameters. Then, once the best model with the fixed hyperparameters had been selected, hyperparameters were selected for the CTC beam search using just that model and its predictions on the development set. The number of samples used for early stopping was chosen to be 8 based on offline evaluation of two blocks collected prior to any real-time test blocks.

Real-time implementation details for the 1024-word-General sentence set: To improve decoding rates, it was reasoned that the decoding model should predict a sentence as soon as the participant stopped attempting to say it, rather than waiting for a fixed window which could be composed of much silence for shorter utterances. Hence, starting 1.9 seconds after the go-cue and every 800 ms thereafter, the RNN was run on all the neural data available. It was then evaluated if the probability of the silence or blank token in the final 8 model outputs (corresponding to 960 ms of neural data) was on average greater than 0.8875. If this threshold was exceeded, then trial ended, the beam-search was run over the model's predictions at each timestep, and the most likely resulting sentence was kept as the final prediction. In the case that the model was fed over 8 seconds of neural data, then the trial was automatically ended. However, this only occurred for 2 of the 250 real-time test trials, demonstrating the effectiveness of the early stopping strategy.

During real-time testing, due to an error a lexicon was used that was missing two words (the final two lines of the lexicon containing pronunciations for the words “pen” and “self” were not present). However, for all trials, it was confirmed that the prediction obtained online with this 1,022 word lexicon perfectly matched the prediction of an offline simulation where the full 1,024 word lexicon was used.

Differences for the 50-phrase-AAC sentence set and the 529-phrase-AAC sentence set: Here any differences (or lack thereof) in model training used for 50-phrase-AAC and the 529-phrase-AAC sentence sets are denoted. Most differences are because the models were developed prior to the 1024-word-General RNN model. Differences are denoted below:
Data preparation: For models used for evaluation of 50-phrase-AAC and 529-phrase-AAC, models were trained using the first 3,956 529-phrase-AAC trials, and first 3,900 330 50-phrase-AAC trials. For the 50-phrase-AAC sentence set, the model was trained using a fixed window from [−1, 3] seconds, and for the 529-phrase-AAC sentence set, a model was trained using a fixed window from [−1, 5.5] seconds. For the 529-phrase-AAC sentence set, the most recent 500 trials from the 50-phrase-AAC sentence set were used to augment the data. The same 95% training set, 5% development set split was used. The neural data was normalized to have an (2-norm of 1 across channels as in previous work [3], rather than across timesteps.
CTC loss: The same loss was used.
Model architecture: The same underlying architecture was used, just with different hyperparameters, which were also found via hand-tuning on evaluation data prior to when the evaluation blocks were collected. The full set of hyperparameters are detailed in Table 12.
Data augmentation: No data augmentations were used during training of the RNN models used with 50-phrase-AAC and 529-phrase-AAC sentence sets.
Optimization: For the AAC sentence sets, the Adam Optimizer was used with a learning rate of 1e-3, and β₁=0.9, β₂=0.999. Model gradients were clipped to have a norm of 1 across the batch, and a batch size of 32 was used.
CTC Beam search and hyperparameters: For the AAC sentence sets, α=3.23, β=−0.26 and B=100 were used as beam search hyperparameters during training and offline evaluation. For each set, a custom n-gram language model was used with n=5 that was trained on each restricted set using kenlm. The hyperparameters were the default hyperparameters for the CTC beam search from torchaudio.
Other: Due to limitations in the amount of data, it was found to be beneficial to initialize the 529-phrase-AAC model with the model trained on the full set of the 50-phrase-AAC sentence set, and an additional 500 samples were also used from the 50-phrase-AAC sentence set during training. Hence, model parameters were optimized for the 529-phrase-AAC sentence set after a model had been trained on the 50-phrase-AAC sentence set.

Simulated evaluation of performance on the 50-phrase-AAC sentence set and the 529-phrase-AAC sentence set: The same blocks were used for evaluation of the synthesis models to evaluate text decoding performance on the 50-phrase-AAC sentence set and the 529-phrase-AAC sentence set. For the 529-phrase-AAC sentence set and the 50-phrase-AAC sentence set only the model tended to hold its last prediction and consistently output it within the last 3 samples, which is prone to happen given the CTC-loss trains the bidirectional network to output any valid sequence of phones regardless of its timing. This did not occur with the 1024-word-General sentence set however, likely since there were more distinct periods of silence in the middle of the sentence productions that encouraged the model to predict silence with more temporal precision. Hence, it was checked if a model had predicted silence or the blank token with a probability >88.75% in the 8 samples prior to the last 3 samples. It was decided to use the delay of 3 samples using a held out block recorded after all train and evaluation blocks were recorded. In the offline scenario, the model was first checked if it had completed its prediction 2.2 seconds after the go-cue, and then every 350 ms after that, or until 5.5 seconds had elapsed since the go cue. Once this occurred, the beam-search was ran and the most likely prediction was kept as the final sentence.

### 1.8. METHOD 3: DECODING NATO CODE WORDS AND HAND-MOTOR MOVEMENTS

Data preparation: The high-gamma and low-frequency signals were used as described in previous work [3], streamed from the real-time system at 200 Hz. Then, the neural activity was downsampled by a factor of 6. Then, the neural activity was normalized to have an l₂ norm of 1 across all channels at each timestep for each set of features.

A window of neural activity was used from [−2, 4] seconds relative to the go-cue during training, and temporal jittering was used and the data augmentations used in text decoding and in previous work [3] was used to make the model more robust to variation in the participant's timing.

The training data was split into a train set and development set by using the last 40 trials of neural activity as the development set, and using the remaining data as the training set. The decoding model was evaluated only on real-time data which had not yet been collected when models were trained.

During evaluation with the development set and during real-time blocks, a window of neural activity from [−1, 3] seconds was used relative to the go-cue for prediction.

RNN architecture: The same architecture described in previous work [3] was use, which consists of a 1-D convolutional layer, followed by multiple layers of bidrectional GRUs. Then, the last hidden state of the final GRU layer was taken, and it was passed through a linear layer followed by the softmax activation function, which produces the probability across the 30 targets (the 26 NATO code words+4 hand-motor targets). During training, dropout between each layer was applied except for between the final GRU layer and the linear layer. The full model hyperparameters, including the hidden units for each layer and dropout rate are in Table 13. The hyperparameters used were the best hyperparameters from [3].

Data augmentations: The same data augmentations and data augmentation parameters as used in previous work [3] were use. The augmentations are described in Method 2.

Optimization: To begin model training, model weights were first loaded from previous experiments that were trained on a participant with a lower density grid, doing a task where the 26 NATO code words and a single hand-motor command were decoded. Hence, the first layer was replaced to accommodate for the different number of channels with the new participant's grid, and replaced the final layer to account for the increased number of classes. Then, the model was trained to minimize cross-entropy loss between the models predictions and the training labels. The Adam Optimizer was used to update model parameters, with a learning rate of 5e-4, batch size of 16, and the default Adam parameters β₁=0.9, β₂=0.999, ϵ=1ϵ-8 [82].

Model evaluation and early stopping: During training, the model with the best accuracy on the held out development set was kept track of and the model with the best accuracy was saved. If accuracy did not improve for 35 epochs, then model training ended, and that model was used as the final model.

Model Ensembling: Starting 40 days after implantation, model ensembling was used as in previous experiments in order to improve model predictions [3]. Prior to this date, only one model's predictions were used during real-time decoding. This meant the training procedure was run 10 times to optimize models with 10 different random initializations. This yielded an ensemble of 10 models which were used during real-time prediction, the probability of the 10 models were averaged to get the final probability across the 30 classes.

### 1.9. METHOD 4: SPEECH SYNTHESIS

### 1.9.1. Modeling

Decoding of discrete speech units: As described above, a sequence of discrete speech sound units was generated for each utterance by passing a basis acoustic waveform through a pre-trained HuBERT model [13]. High-gamma and low frequency features were then used from neural data to decode a sequence of discrete speech units sampled at 50 Hz.

HuBERT unit-to-speech vocoder: A two step process was applied to synthesize speech from decoded sequences of discrete speech units, which were adapted from the discrete-unit-based synthesizer introduced in Generative Spoken Language Model (GSLM) [83]. The synthesizer first applies a Tacotron2 model that generates a speech mel-spectrogram from a sequence of discrete units. This is then followed by a WaveGlow vocoder that outputs a speech waveform from the mel-spectrogram. The pretrained HuBERT, Tacotron2 and WaveGlow models were obtained from fairseq from the following link: https: (and)//dl.fbaipublicfiles. (and) com/textless_nlp/gslm/hubert/tts_440 km100/tts_checkpoint_best.pt, wherein the (and) and surrounding spaces are omitted.

Model architecture: A neural network was trained to predict sequences of discrete HuBERT units from neural activity. This neural network consists of a 1-D convolutional layer followed by a 3-layer bidirectional gated-recurrent units (GRUs). The hidden state of the final GRU is then passed into a 1D transpose convolutional layer, which upsamples the hidden representation back to the original sampling rate. The output feature dimension of the transpose convolutional layer is 101, which corresponds to the logits of the 100 HuBERT units and an additional blank token needed for CTC decoding. Hyperparameters of the network are listed in Table 14.

Training and optimization: As for text decoding, the synthesizing network was trained with a fixed duration window of neural activity. For decoding with the 529-phrase-AAC and 50-phrase-AAC a window of −0.5 to 4.62 seconds was used relative to the go cue. For the 1024-word-General sentence set a longer window of 0 to 7.5 seconds was used relative to the go cue. For each utterance in the training sets, the discrete units were computed from a basis speech waveform. During training, the CTC loss was used to train the neural network. SpecAugment was applied during training as a data augmentation for the ECoG data.

The decoder was trained with the Adam optimizer [82]. An initial learning rate of 1e-4 and a multistep learning rate scheduler with a y of 0.5 and milestones at 40000, 80000, 120000, and 1600000 iterations were used. β₁=0.5 and β₂=0.9 were used. A batch size of 64 was used for 529-phrase-AAC and 1024-word-General sets and batch size of 16 was used for 50-phrase-AAC set.

CTC decoding: After obtaining the output logits from the neural decoding model, the greedy CTC-decoding algorithm was applied to determine the final decoded discrete units. The token was first computed with the highest probability at each timepoint. Consecutively repeating tokens were collapsed into one token and then blank tokens were removed, producing the final decoded sequence of units.

Hyperparameter search: Hyperparameters were manually searched for including the number of hidden units in the model, dropout rate, number of layers, kernel size, feature dimension, and stride size. Hyperparameters were chose based on the decoded unit error rate for the held-out development set.

Perceptual assessment: Perceptual assessments were designed using a crowd-sourcing platform (Amazon Mechanical Turk), where each trial from each synthesis test set was assessed by 12 workers (except for 3 of the 500 trials, in which only 11 workers completed their evaluations). Each evaluation consisted of playback of the decoded waveform and workers were then asked to transcribe what they heard. The precise instructions were as follows:
1. Please listen to the audio and write down what you hear. Many of the clips may be difficult to hear. If this is the case, write whatever words you are able to make out, even if it does not form a complete expression. If you are not sure about a word, please only include your guess in your transcription if you feel that you are over 50% confident that your guess is correct. Otherwise, exclude the guess from your transcription. If you cannot make out any words, leave the entry blank. You may listen as many times as needed.

The decoded avatar animations (for the direct approach and the acoustic approach) were also evaluated in the absence of speech. Each decoded animation was assessed by 6 unique evaluators. Each evaluation consisted of playback of the decoded animation (with no audio) and textual presentation of the target (ground-truth) sentence and a randomly chosen other sentence from the same sentence set. Workers were presented with the ground-truth phrase and a randomly chosen phrase from the test set. Evaluators were instructed to identify the phrase that they thought the avatar was trying to say. The precise instructions were as follows:
1. This is a lip-reading task. First, please look at the two phrase options. Then watch the silent video clip of the avatar. Choose the phrase closest to what you were able to lip-read. You may watch the video as many times as needed. Click the submit button after selection to move to the next HIT.

For all perceptual metrics, the median worker response accuracy was taken as the accuracy for that trial.

### 1.10. METHOD 5: AVATAR

Virtual environment and avatar animation system: To animate the avatar during real-time decoding, Speech Graphics' “SG Com” audio-driven animation system was used. This system takes a waveform, applies a speech-to-gesture model [28], and then animates these articulatory gestures. During audio-visual synthesis collection, the decoded speech waveform was taken and passed through this system to generate the avatar animation in sync with the audio waveform. The audio waveform was delayed by 200 ms to improve audio-visual synchronization.

For direct avatar decoding, SG Com provided a custom SG Com build that instead takes articulatory gestures as input, allowing bypassing of the dependency on a speech-to-gesture model and speech waveform and instead the feeding in directly of decoded articulatory gestures. However, speech-to-gesture component of SG Com was used to generate reference articulatory gestures for targets during direct avatar decoding.

A virtual environment was designed using Unreal Engine 4.26 to hold the MetaHuman characters (developed by Epic Games (Cary, North Carolina) for the Unreal Engine). The participant was shown the full range of over 40 MetaHuman characters and she chose which one she preferred. She selected the character “Vivian” which was used for subsequent real-time experiments and offline rendering. The virtual scene consists of a simple camera, a series of spotlights, the “Vivian” MetaHuman's character, and a black background wall, see FIG. 29. The virtual environment ran on a Microsoft Surface Book 3 which was connected to the participant's monitor to display the avatar.

A custom C++ extension was built to stream decoded features to the avatar. This extension waits to receive data (articulatory gestures or audio) from an ethernet cable connected to the real-time decoding PC. Articulatory gestures (used for gesture demos) or audio in 10 ms chunks were streamed from the real-time PC using the Transmission Control Protocol.

Continuous articulatory gesture decoding decoding: VQ-VAE: In order to train a model to predict articulatory gestures using the CTC loss, the articulatory gestures were first discretized.

This was done using a VQ-VAE [85]. The VQ-VAE was trained to minimize the objective in [85], with β=2, and with additional weighting of λ_{jaw}=20 on the reconstruction loss (mean-squared-error) of the jaw opening gesture, and λ_{visuallysalient}=5 on the tongue body raise, tongue advance, tongue retraction, tongue tip raise, lip rounding, and lip retraction gestures, to emphasize the most visually salient avatar features and jaw over other features, which effectively had a weight of 1. These were selected by grid searching over the weights [5, 10, 20] for both λ_{jaw} and λ_{visuallysalient}. The parameters were selected based on the development set correlation of the 534 reconstructed vs reference jaw and visually salient articulatory gestures.

The architecture of the VQ-VAE was an encoder with 3 1-D convolutional layers, filter size 40, kernel size (KS) 4, stride 2. The ReLU activation followed layers 2 and 3. After that, a 1-d convolutional layer with KS 1 and stride 1 was applied. A codebook was used with 40 1-dimensional vectors, which were initialized with the distribution of (U[−1,1]/dₑₙₑ), where dₑₙₑ is the dimensionality of the codebook, in this case 40. The decoder consisted of a 1-dimensional convolution with 40 kernels with size 1 and stride 1, followed by 3 layers of 1d transpose convolutions with KS=4, stride-2, that upsampled the representations back to the original sampling rate of 100 Hz. The layers had 40, 40 and 16 kernels, respectively. The dimensionality of the VQ-VAEs hidden units and codebooks were found via manual tuning when evaluating the reconstruction quality of the full system (VQ-VAE and CTC decoder), rather than just the VQ-VAE's reconstruction.

The VQ-VAE was trained using the AdamW optimizer [80], with a learning rate of 1e-3, weight decay of 1e-2, β₁=0.9, and β₂=0.999. A batch size of 32 was used, and the l₂ norm of the gradient was clipped to be 1 across all samples in the batch. For training, all trials used for evaluation with the 1024-word-General sentence set were first held out from being used as training or dev set data. 90% of the remaining data was then selected (any trial that was not used for evaluation with the 1024-word-General sentence set) as training data, and then 10% of the data was used for early stopping. Models were early stopped when the test loss for that epoch did not improve over the loss from the previous epoch by at least 1e-6, which occurred for the model after 636 total epochs of training. The VQ-VAE parameters were then frozen and were not further trained as part of the CTC decoding process.

CTC decoding: A neural network decoder was next trained to predict VQ-VAE units based on neural activity. To do this, the units were first encoded as a discrete sequence, and the token 0 was reserved for the blank token used in CTC decoding.

The neural data was preprocessed identically to the preprocessing done for text decoding with the 1024-word-General sentence set.

An architecture was then used nearly identical to that used for text decoding, hence a small set of hyperparameters close to those used in text decoding were manually searched over. Identical training procedures were used as used in text decoding for the 1024-word-General set, however temporal jittering was not used during training, and instead a fixed window from [−1, 8] seconds was used relative to the go cue for the 1024-word-General sentence set, and [−1, 6] seconds was used relative to the go-cue for the 50-phrase-AAC sentence set and the 529-phrase-AAC sentence set. However, all other data augmentations were used with the same values as used for text decoding.

Evaluation: During offline evaluation, the same full windows of activity were used as used for training: from [−1, 8] seconds relative to the go cue for the 1024-word-General sentence set dataset, and [−1, 6] seconds relative to the go-cue for the 50-phrase-AAC sentence set and the 529-phrase-AAC sentence set. The decoder was able to output the blank token during silence, thus accounting for variations in production length. A greedy search was used over the models probabilities at each timestep to get the most likely series of VQ-VAE units, and collapsed across repeated units. This set of units was then passed in the VQ-VAE's decoder (which was not updated as part of further training) to produce articulatory gestures.

To evaluate how facial features from healthy speakers compare with those decoded from the avatar, the dlib software package was used to extract 72 facial keypoints for each frame in avatar-rendered and healthy speaker videos (30 frames per second) of all three sentence sets.

For the direct-approach, these videos were extracted by running the decoded articulatory gestures through the avatar animation system offline using Speech Graphics' gesture-animation system. All the gestures were then concatenated and the concatenated gesture animation was played while screen recording to generate the full video of all decoded trials. For the acoustic-approach, the videos captured during real-time audio-visual synthesis were used. Here the videos were cropped to only include the avatar portion of the screen and environment.

For the healthy speakers, 8 English speakers (6 female and 2 male) were recruited to record themselves while speaking sentences from the 1024-word-General sentence set. Labels of sentences to read and the following instructions were given to them (with edits for brevity; they were also provided with a secure location to transfer their recorded data to):
Get familiar with the labels. The first number is the block number. The second number is the trial number in order. During recording, you will record one block at a time.
   Record yourself speaking the sentences using the front-facing webcam:
      1. Make sure your face is in view, at a minimum, up until the lower part of your eyes and showing the full jaw, even when mouth is open. When the videos are processed, the region of interest will be cropped to. Make sure to remove any glasses, ensure camera is clear, and that you have still background.
      2. Make sure you are recording in a quiet environment. If there is talking or noise in the background, please restart the block.
      3. Record each block, reading each sentence back to back in order. Pause for roughly 1-2 seconds in between sentences. Make sure to close mouth in between readings and try to minimize head and shoulder movement (i.e. keep them in roughly the same place).
      4. Save each video and upload to a secure location.

For the healthy speakers and acoustic approach, the videos were segmented according to a manually selected acoustic onset and offset threshold and then trimmed around the video using a window of [−1, 0.3] seconds. For the direct approach, the videos were segmented afterwards by automatically splicing the videos according to the length of the gestures, then included 1.5 seconds of padding at the beginning and end of the gestures. One pseudoblock of data was then used to determine closer trimming landmarks based on visual analysis of the dlib trajectories. For all three approaches, gestural padding was not included in the dlib analyses.

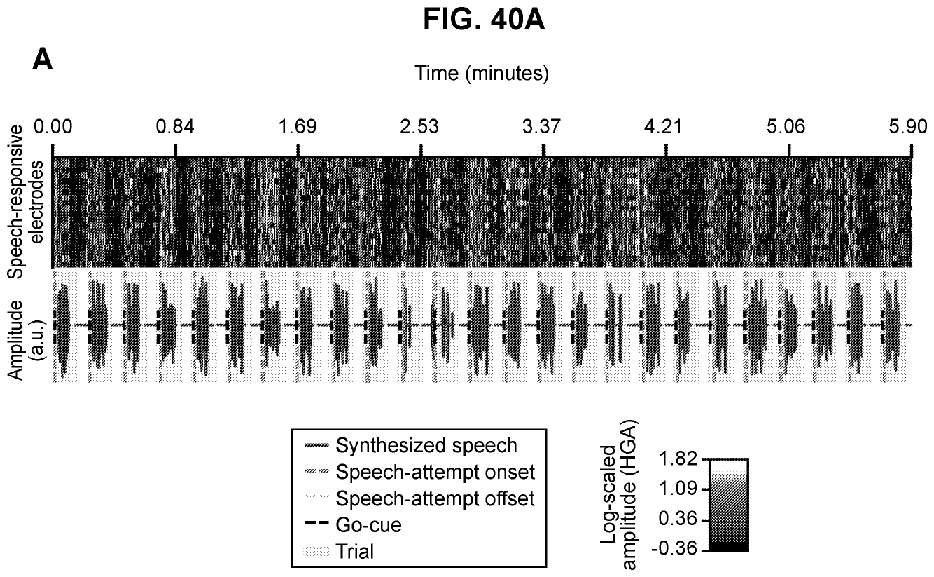

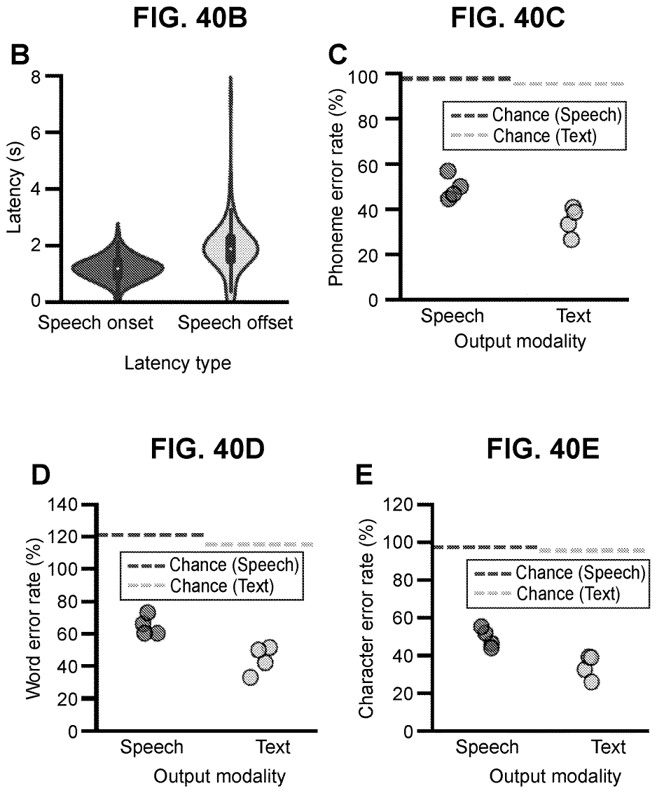

To extract trajectories, the Euclidean distance between key points was used rather than the value of a single keypoint in order to account for head movements, scale, and rotation. The jaw movement was evaluated by extracting the distance between the keypoint at the bottom of the jaw and the tip of the nose (keypoints 33 and 8). Lip aperture was evaluated as the distance between the keypoint at the top and bottom of lips (keypoints 51 and 63), and mouth width was evaluated as the distance between the key points at either corners of the mouth (key points 54 and 48). The keypoints are in FIG. 40.

Expression Decoding: To decode expressions the same model architecture was used as in NATO code-word classification. A single change was made to increase dropout to 0.8, to prevent over-fitting on limited training data. The weights of the model were initialized, excluding the final readout layer, with a pre-trained NATO code-word classification model with the same hyperparameters as described in Table 13 (trained on NATO blocks prior to the start of expression data collection, 1222 samples). The same data augmentations described in (Text decoding: Data augmentations) was used with parameters that were previously found to be effective [3]. The model was trained using the Adam optimizer with learning rate of 5e-4 to perform stochastic batch gradient decent. A batch size of 16 was used during training, given smaller amounts of training examples. The model loss and accuracy were evaluated after each training epoch. For each of 15 CV folds, 10% of the training data was reserved as a validation set. 10 models per fold were then fit to ensemble predictions on the held out test set. Training if was early stopped accuracy did not improve for 20 epochs on the validation set and the model with best accuracy on the validation set was kept.

Articulatory-movement decoding: A small grid-search was performed over the hyperparameters for the number of layers used in the network, the dropout rate, and the number of hidden units. The final 40 samples collected were held out as an evaluation set during the hyperparameter search. The set was not used for model training or evaluation after hyperparameters were selected. A grid search was done over the values [128, 256, 512] for the number of hidden units, [0.4, 0.5, 0.6] for the dropout rate, and [2, 3, 4] as the number of layers. It was found the optimal values were 512, 0.4, and 2, respectively. Model performance was then evaluated using these hyperparameters across 10 held-out folds using the remaining data (800 trials).

The neural network was trained using the Adam optimizer with a learning rate of 1e-3, β₁=0.9, β₂=0.999, and a batch size of 32. The model loss was evaluated after each training epoch. 10% of the data was used as the test set. From the remaining 90%, 90% of the data was used to train the model, and then 10% of the data was used as an evaluation set to early-stop the model. If accuracy did not improve for 20 epochs, training stopped and the model with the best evaluation set accuracy was used as the final model.

Avatar implementation during articulatory-movement and expression decoding: Speech Graphics also provided target gestures for the orofacial movements and expressions tasks. After classifying the most likely movement or expression, they were sent to the Microsoft Surface Book 3 in a streaming fashion. For each emotion, the participant chose which expression she would like to express from 10 variations. The “high” expression corresponded to a maximally intense expression, where as “medium” and “low” were respectively ⅔ and ⅓ the intensity of the “strong” expression.

Perceptual assessment: The precise instructions used during the binary-choice perceptual assessment of the avatar videos were: This is a lip-reading task. First, please look at the two phrase options. Then watch the silent video clip of the avatar. Choose the phrase closest to what you were able to lip-read. You may watch the video as many times as needed. Click the submit button after selection to move to the next HIT.

### 1.11. REFERENCES

The numbering related to the following references apply with respect to the experimental results presented in Example 1:
[1] Peters, B. et al. Brain-Computer Interface Users Speak Up: The Virtual Users' Forum at the 2013 International Brain-Computer Interface Meeting. Arch. Phys. Med. Rehabil. 96, S33-S37 (2015).
[2] Moses, D. A. et al. Neuroprosthesis for Decoding Speech in a Paralyzed Person with Anarthria. N. Engl. J. Med. 385, 217-227 (2021).
[3] Metzger, S. L. et al. Generalizable spelling using a speech neuroprosthesis in an individual with severe limb and vocal paralysis. Nat. Commun. 13, 6510 (2022).
[4] Beukelman, D. R., Mirenda, P., & others. Augmentative and alternative communication. (Paul H. Brookes Baltimore, 1998).
[5] Graves, A., Fernández, S., Gomez, F. & Schmidhuber, J. Connectionist temporal classification: labelling unsegmented sequence data with recurrent neural networks. in Proceedings of the 23rd international conference on Machine learning—ICML '06 369-376 (ACM Press, 2006). doi:10.1145/1143844.1143891.
[6] Watanabe, S., Delcroix, M., Metze, F. & Hershey, J. R. New era for robust speech recognition: exploiting deep learning. (Springer-Verlag, 2017).
[7] Vansteensel, M. J. et al. Fully Implanted Brain-Computer Interface in a Locked-In Patient with ALS. N. Engl. J. Med. 375, 2060-2066 (2016).
[8] Pandarinath, C. et al. High performance communication by people with paralysis using an intracortical brain-computer interface. eLife 6, 1-27 (2017).
[9] Willett, F. R., Avansino, D. T., Hochberg, L. R., Henderson, J. M. & Shenoy, K. V. High-performance brain-to-text communication via handwriting. Nature 593, 249-254 (2021).
[10] Hénaff, O. J. et al. Data-Efficient Image Recognition with Contrastive Predictive Coding. ArXiv190509272 Cs (2020).
[11] Angrick, M. et al. Speech synthesis from ECoG using densely connected 3D convolutional neural networks. J. Neural Eng. 16, 036019 (2019).
[12] Anumanchipalli, G. K., Chartier, J. & Chang, E. F. Speech synthesis from neural decoding of spoken sentences. Nature 568, 493-498 (2019).
[13] Hsu, W.-N. et al. HuBERT: Self-Supervised Speech Representation Learning by Masked Prediction of Hidden Units. IEEEACM Trans. Audio Speech Lang. Process. 29, 3451-3460 (2021).
[14] Cho, C. J., Wu, P., Mohamed, A. & Anumanchipalli, G. K. Evidence of Vocal Tract Articulation in Self-Supervised Learning of Speech. Preprint at https://doi.org/10.48550/arXiv.2210.11723 (2022).
[15] Lakhotia, K. et al. Generative Spoken Language Modeling from Raw Audio. (2021) doi:10.48550/ARXIV.2102.01192.
[16] Prenger, R., Valle, R. & Catanzaro, B. Waveglow: A Flow-based Generative Network for Speech Synthesis. in ICASSP 2019-2019 IEEE International Conference on Acoustics, Speech and Signal Processing (ICASSP) 3617-3621 (IEEE, 2019). doi:10.1109/ICASSP.2019.8683143.
[17] Kubichek, R. Mel-cepstral distance measure for objective speech quality assessment. in Proceedings of IEEE Pacific Rim Conference on Communications Computers and Signal Processing vol. 1 125-128 (IEEE, 1993).
[18] Yamagishi, J. et al. Thousands of Voices for HMM-Based Speech Synthesis-Analysis and Application of TTS Systems Built on Various ASR Corpora. IEEE Trans. Audio Speech Lang. Process. 18, 984-1004 (2010).
[19] Wolters, M. K., Isaac, K. B., Renals, S. Evaluating speech synthesis intelligibility using Amazon Mechanical Turk. Proc 7th ISCA Workshop Speech Synth. SSW 7 136-141 (2010).
[20] Mehrabian, A. Silent messages: implicit communication of emotions and attitudes. (1981).
[21] Jia, J., Wang, X., Wu, Z., Cai, L. & Meng, H. Modeling the correlation between modality semantics and facial expressions. in Proceedings of The 2012 Asia Pacific Signal and Information Processing Association Annual Summit and Conference 1-10 (2012).
[22] Sadikaj, G. & Moskowitz, D. S. I hear but I don't see you: Interacting over phone reduces the accuracy of perceiving affiliation in the other. Comput. Hum. Behav. 89, 140-147 (2018).
[23] Sumby, W. H. & Pollack, I. Visual Contribution to Speech Intelligibility in Noise. J. Acoust. Soc. Am. 26, 212-215 (1954).
[24] Chartier, J., Anumanchipalli, G. K., Johnson, K. & Chang, E. F. Encoding of Articulatory Kinematic Trajectories in Human Speech Sensorimotor Cortex. Neuron 98, 1042-1054.e4 (2018).
[25] Bouchard, K. E., Mesgarani, N., Johnson, K. & Chang, E. F. Functional organization of human sensorimotor cortex for speech articulation. Nature 495, 327-332 (2013).
[26] Carey, D., Krishnan, S., Callaghan, M. F., Sereno, M. I. & Dick, F. Functional and Quantitative MRI Mapping of Somatomotor Representations of Human Supralaryngeal Vocal Tract. Cereb. Cortex 27, 265-278 (2017).
[27] Mugler, E. M. et al. Differential Representation of Articulatory Gestures and Phonemes in Precentral and Inferior Frontal Gyri. J. Neurosci. 4653, 1206-18 (2018).
[28] Berger, M. A., Hofer, G. & Shimodaira, H. Carnival—Combining Speech Technology and Computer Animation. IEEE Comput. Graph. Appl. 31, 80-89 (2011).
[29] Oord, A. van den, Vinyals, O. & Kavukcuoglu, K. Neural Discrete Representation Learning. Preprint at http://arxiv.org/abs/1711.00937 (2018).
[30] King, Davis E. Dlib-ml: A Machine Learning Toolkit. J. Mach. Learn. Res. 10, 1755-1758 (2009).
[31] Eichert, N., Papp, D., Mars, R. B. & Watkins, K. E. Mapping Human Laryngeal Motor Cortex during Vocalization. Cereb. Cortex 30, 6254-6269 (2020).
[32] Breshears, J. D., Molinaro, A. M. & Chang, E. F. A probabilistic map of the human ventral sensorimotor cortex using electrical stimulation. J. Neurosurg. 123, 340-349 (2015).
[33] Muller, L. et al. Thin-film, high-density micro-electrocorticographic decoding of a human cortical gyrus. in 2016 38th Annual International Conference of the IEEE Engineering in Medicine and Biology Society (EMBC) 1528-1531 (IEEE, 2016). doi:10.1109/EMBC.2016.7591001.
[34] Makin, J. G., Moses, D. A. & Chang, E. F. Machine translation of cortical activity to text with an encoder-decoder framework. Nat. Neurosci. 23, 575-582 (2020).
[35] Duraivel, S. et al. Accurate speech decoding requires high-resolution neural interfaces. http://biorxiv.org/lookup/doi/10.1101/2022.05.19.492723 (2022) doi:10.1101/2022.05.19.492723.
[37] Guenther, F. H. Neural Control of Speech. (MIT Press, 2016).
[38] Umeda, T., Isa, T. & Nishimura, Y. The somatosensory cortex receives information about motor output. Sci. Adv. 5, eaaw5388 (2019).
[39] Murray, E. A. & Coulter, J. D. Organization of corticospinal neurons in the monkey. J. Comp. Neurol. 195, 339-365 (1981).
[39] Arce, F. I., Lee, J.-C., Ross, C. F., Sessle, B. J. & Hatsopoulos, N. G. Directional information from neuronal ensembles in the primate orofacial sensorimotor cortex. Am. J. Physiol.-Heart Circ. Physiol. (2013) doi:10.1152/jn.00144.2013.
[40] Rousseau, M.-C. et al. Quality of life in patients with locked-in syndrome: Evolution over a 6-year period. Orphanet J. Rare Dis. 10, 88-88 (2015).
[41] Felgoise, S. H., Zaccheo, V., Duff, J. & Simmons, Z. Verbal communication impacts quality of life in patients with amyotrophic lateral sclerosis. Amyotroph. Lateral Scler. Front. Degener. 17, 179-183 (2016).
[42] Huggins, J. E., Wren, P. A. & Gruis, K. L. What would brain-computer interface users want? Opinions and priorities of potential users with amyotrophic lateral sclerosis. Amyotroph. Lateral Scler. 12, 318-324 (2011).
[43] Berezutskaya, J. et al. Direct Speech Reconstruction from Sensorimotor Brain Activity with Optimized Deep Learning Models. http://biorxiv.org/lookup/doi/10.1101/2022.08.02.502503 (2022) doi:10.1101/2022.08.02.502503.
[44] Conant, D. F., Bouchard, K. E., Leonard, M. K. & Chang, E. F. Human sensorimotor cortex control of directly-measured vocal tract movements during vowel production. J. Neurosci. 38, 2382-17 (2018).
[45] Ajiboye, A. B. et al. Restoration of reaching and grasping movements through brain-controlled muscle stimulation in a person with tetraplegia: a proof-of-concept demonstration. The Lancet 389, 1821-1830 (2017).
[46] Cajigas, I. et al. Implantable brain-computer interface for neuroprosthetic-enabled volitional hand grasp restoration in spinal cord injury. Brain Commun. 3, fcab248 (2021).
[47] Collinger, J. L. et al. High-performance neuroprosthetic control by an individual with tetraplegia. The Lancet 381, 557-564 (2013).
[48] Brumberg, J. S., Pitt, K. M. & Burnison, J. D. A Noninvasive Brain-Computer Interface for Real-Time Speech Synthesis: The Importance of Multimodal Feedback. IEEE Trans. Neural Syst. Rehabil. Eng. 26, 874-881 (2018).
[49] Sadtler, P. T. et al. Neural constraints on learning. Nature 512, 423-426 (2014).
[50] Chiang, C.-H. et al. Development of a neural interface for high-definition, long-term recording in rodents and nonhuman primates. Sci. Transl. Med. 12, eaay4682 (2020).
[51] Crone, N. E., Miglioretti, D. L., Gordon, B. & Lesser, R. P. Functional mapping of human sensorimotor cortex with electrocorticographic spectral analysis. II. Event-related synchronization in the gamma band. Brain 121, 2301-2315 (1998).
[52] Moses, D. A., Leonard, M. K. & Chang, E. F. Real-time classification of auditory sentences using evoked cortical activity in humans. J. Neural Eng. 15, (2018).
[53] Bird, S. & Loper, E. NLTK: The Natural Language Toolkit. in Proceedings of the ACL Interactive Poster and Demonstration Sessions 214-217 (Association for Computational Linguistics, 2004).
[54] Danescu-Niculescu-Mizil, C. & Lee, L. Chameleons in imagined conversations: A new approach to understanding coordination of linguistic style in dialogs. Preprint at https://doi.org/10.48550/arXiv.1106.3077 (2011).
[55] Virtanen, P. et al. SciPy 1.0: fundamental algorithms for scientific computing in Python. Nat. Methods 17, 261-272 (2020).
[56] Park, K. & Kim, J. g2pE. (2019).
[57] Graves, A., Mohamed, A. & Hinton, G. Speech recognition with deep recurrent neural networks. in International Conference on Acoustics, Speech, and Signal Processing 6645-6649 (2013). doi:10.1109/ICASSP.2013.6638947.
[58] Hannun, A. et al. Deep Speech: Scaling up end-to-end speech recognition. Preprint at https://doi.org/10.48550/arXiv.1412.5567 (2014).
[59] Paszke, A. et al. Automatic differentiation in PyTorch.
[60] Collobert, R., Puhrsch, C. & Synnaeve, G. Wav2Letter: an End-to-End ConvNet-based Speech Recognition System. ArXiv160903193 Cs (2016).
[61] Yang, Y.-Y. et al. Torchaudio: Building Blocks for Audio and Speech Processing. in ICASSP 2022-2022 IEEE International Conference on Acoustics, Speech and Signal Processing (ICASSP) 6982-6986 (2022). doi:10.1109/ICASSP43922.2022.9747236.
[62] Jurafsky, D. & Martin, J. H. Speech and language processing: an introduction to natural language processing, computational linguistics, and speech recognition. (Pearson Education, Inc., 2009).
[63] Kneser, R. & Ney, H. Improved backing-off for M-gram language modeling. in 1995 International Conference on Acoustics, Speech, and Signal Processing vol. 1 181-184 (IEEE, 1995).
[64] Heafield, K. KenLM: Faster and Smaller Language Model Queries.
[65] Panayotov, V., Chen, G., Povey, D. & Khudanpur, S. Librispeech: An ASR corpus based on public domain audio books. in 2015 IEEE International Conference on Acoustics, Speech and Signal Processing (ICASSP) 5206-5210 (2015). doi:10.1109/ICASSP.2015.7178964.
[66] Ito, Keith & Johnson, Linda. The LJ Speech Dataset. (2017).
[67] Oord, A. van den et al. WaveNet: A Generative Model for Raw Audio. ArXiv160903499 Cs (2016).
[68] Ott, M. et al. fairseq: A Fast, Extensible Toolkit for Sequence Modeling. Preprint at https://doi.org/10.48550/arXiv.1904.01038 (2019).
[69] Park, D. S. et al. SpecAugment: A Simple Data Augmentation Method for Automatic Speech Recognition. Interspeech 2019 2613-2617(2019) doi:10.21437/Interspeech.2019-2680.
[70] Lee, A. et al. Direct speech-to-speech translation with discrete units. Preprint at https://doi.org/10.48550/arXiv.2107.05604 (2022).
[71] Casanova, E. et al. YourTTS: Towards Zero-Shot Multi-Speaker TTS and Zero-Shot Voice Conversion for everyone. Preprint at http://arxiv.org/abs/2112.02418 (2022).
[72] The most powerful real-time 3D creation tool—Unreal Engine. (2020).
[73] Ekman, P. & Friesen, W. V. Facial Action Coding System. (2019) doi:10.1037/t27734-000.
[74] Gramfort, A. et al. MEG and EEG data analysis with MNE-Python. Front. Neurosci. 7, (2013).
[75] Müllner, D. Modern hierarchical, agglomerative clustering algorithms. Preprint at http://arxiv.org/abs/1109.2378 (2011).
[76] Pedregosa, F. et al. Scikit-learn: Machine Learning in Python. J. Mach. Learn. Res. 12, 2825-2830 (2011).
[77] Waskom, M. seaborn: statistical data visualization. J. Open Source Softw. 6, 3021 (2021).
[78] Seabold, S. & Perktold, J. Statsmodels: Econometric and Statistical Modeling with Python. in 92-96 (2010). doi:10.25080/Majora-92bf1922-011.
[79] Hamilton, L. S., Chang, D. L., Lee, M. B. & Chang, E. F. Semi-automated Anatomical Labeling and Inter-subject Warping of High-Density Intracranial Recording Electrodes in Electrocorticography. Front. Neuroinformatics 11, (2017).
[80] Loshchilov, I. & Hutter, F. Fixing Weight Decay Regularization in Adam. CoRR abs/1711.05101. arXiv: 1711.05101. http://arxiv.org/abs/1711.05101 (2017).
[81] Ney, H., Essen, U. & Kneser, R. On structuring probabilistic dependences in stochastic language modelling. Computer Speech & Language 8, 1-38 (1994).
[82] Kingma, D. P. & Ba, J. Adam: A method for stochastic optimization. arXiv preprint arXiv: 1412.6980 (2014).
[83] Lakhotia, K. et al. On generative spoken language modeling from raw audio. Transactions of the Association for Computational Linguistics 9, 1336-1354 (2021).
[84] Ott, M. et al. fairseq: A Fast, Extensible Toolkit for Sequence Modeling in Proceedings of NAACL-HLT 2019: Demonstrations (2019). 730
[85] Van den Oord, A., Vinyals, O. & Kavukcuoglu, K. Neural Discrete Representation Learning. CoRR abs/1711.00937. arXiv: 1711.00937. http://arxiv.org/abs/1711. 732 00937 (2017).
[86] Salvador, S. & Chan, P. Toward accurate dynamic time warping in linear time and space. Intelligent Data Analysis 11, 561-580 (2007).
[87] Hullett, P. W., Hamilton, L. S., Mesgarani, N., Schreiner, C. E. & Chang, E. F. Human superior temporal gyrus organization of spectrotemporal modulation tuning derived from speech stimuli. The Journal of Neuroscience 36, 2014-2026 (2016).

### Example 2: A Streaming Silent-Speech Neuroprosthesis for Restoring Naturalistic Communication

### 2.1. OVERVIEW

Natural spoken communication happens instantaneously. Speech delays longer than a few seconds can disrupt the natural flow of conversation. This makes it difficult for individuals with paralysis to participate in meaningful dialogue, potentially leading to feelings of isolation and frustration. While speech neuroprostheses aim to restore naturalistic communication, it has yet been unclear whether speech can be synthesized from the brain with low latency, mirroring natural speech production. Here, high-density surface recordings of the speech sensorimotor cortex in a clinical-trial participant with severe paralysis and anarthria were used to drive a continuously streaming naturalistic speech synthesizer. Novel deep-learning transducer models were designed and employed to achieve online large-vocabulary intelligible fluent speech synthesis personalized to the participant's pre-injury voice with neural decoding in 80-ms increments. Offline, the models demonstrated implicit speech-detection capabilities and could continuously decode speech indefinitely, enabling uninterrupted use of the decoder and further increasing speed. The disclosed framework also successfully generalized to other silent-speech interfaces, including single-unit recordings and electromyography. The findings introduce a new speech-neuroprosthetic paradigm to restore naturalistic spoken communication to people with paralysis.

### 2.2. INTRODUCTION

Loss of spoken communication after neurological injury can be debilitating [1-3]; however, a neuroprosthesis that restores naturalistic and embodied communication from the individual's intent to speak would be transformative and significantly improve their quality of life [4-6]. The speech sensorimotor cortex has been shown to encode a rich array of articulatory and speech-production-related information [7-11] that can be used to drive speech decoding in healthy speakers in the form of text or synthesized speech. Translating these findings into speech neuroprostheses that can restore fluent communication in people with severe vocal-tract paralysis has been elusive. This is because synthesizing intelligible speech without vocalization or coordinated articulation is a challenging goal. Individuals who have lost the ability to speak cannot produce an intelligible waveform that can be used as the target during supervised model training, a leading paradigm used in current decoders. Previous demonstrations have shown that it is possible to decode intended speech from neural activity alone, recently expanding to large-vocabulary text decoding followed by text-to-speech to produce the intended phrase [17, 23], or additionally speech-synthesis and accompanying orofacial movements [22]. However, the approaches to output audible speech process an entire window of neural activity corresponding to a speech attempt and wait until the end of the attempted speech production to synthesize speech directly [22,27] or apply text-to-speech synthesis [17,23], causing extended periods of delays which scale linearly with the duration of the speech attempt. From this perspective, these approaches can be considered as running the inference offline and then subsequently playing the offline generated speech waveform back to the participant. A naturalistic speech neuroprosthesis should operate in the same manner as natural spoken communication: as the participant tries to speak, speech is immediately synthesized from their neural activity.

Furthermore, speech delays can hinder the flow of conversation, leading to misunderstandings or frustration for both the speaker and the listener, and can even degrade the perceived quality of the speech [28,29]. Therefore, having a long wait from intent to speak to the produced sound severely limits the natural engagement in communication and decreases the overall communication rate [17,22,23]. Low-latency spoken communication is crucial for maintaining the perceived quality of the conversation [28] and minimizing the frequency of confusions, for example, via interruptions, repeating one's own speech, or unnecessarily long silence periods [30]. Hence, a practical speech neuroprosthesis must continuously synthesize speech from neural data in tandem with low latency from the user's attempt to speak. As persons with severe vocal-tract paralysis may not be able to vocalize, the neuroprosthesis should not rely on audible vocalization at any point during training or inference. Additionally, speech-BCI users should be able to speak using their decoding system continuously and volitionally upon attempting to speak [31]. This capability is crucial as it mirrors the healthy speaker's ability to produce spontaneous speech, enabling speech-BCI users to initiate spoken conversation or respond in real time without external prompts or cues. Lastly, given that articulatory features of neural activity [7-11] and corresponding articulation give rise to intelligible speech in healthy persons, it was hypothesized that any articulatory-recording interface with sufficient spatiotemporal resolution, of which speech neuroprostheses are a subset, should be able to be used to train a model which synthesizes intelligible speech.

### 2.3. RESULTS

### 2.3.1. A Naturalistic Streaming Silent-Speech Decoding System

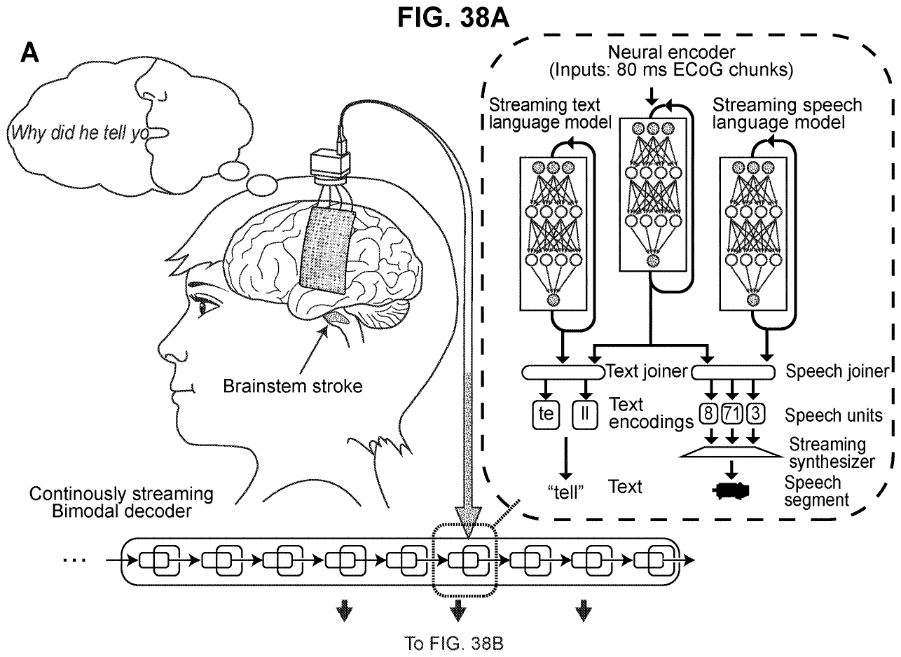

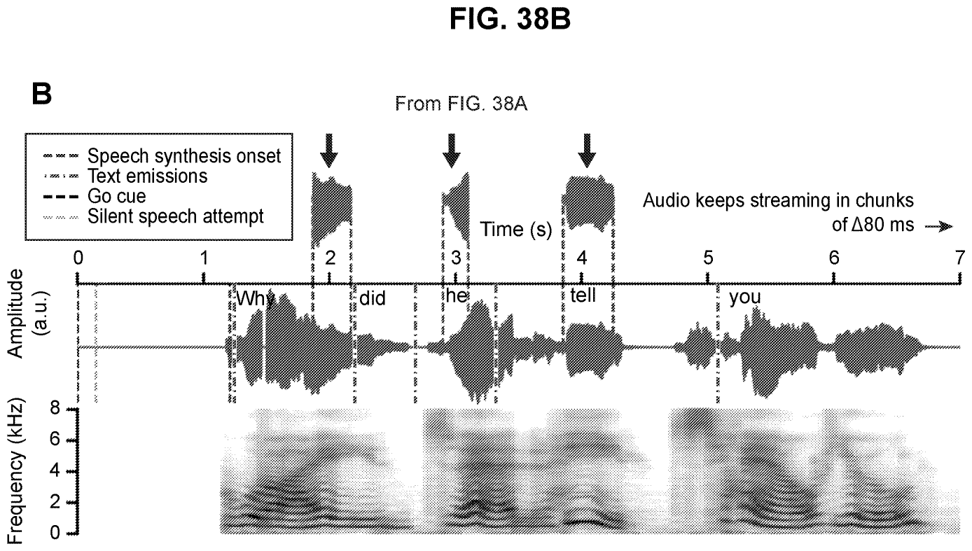

A speech-synthesis neuroprosthesis was designed that enabled a clinical-trial participant (ClinicalTrials.gov; NCT03698149) to naturalistically speak by synthesizing intended speech from neural signals acquired from a 253-channel electrocorticography (ECoG) array implanted over the surface of her speech sensorimotor cortex and a small portion of the temporal lobe (FIG. 38A). To train the system, neural data was recorded as the participant silently attempted to speak individual sentences. The participant was presented with a text prompt on a monitor and was asked to begin silently attempting to speak once a visual go-cue turned green (see Methods). During the online evaluation, the task design was the same. In addition, the synthesized speech was streamed through a nearby analog speaker and decoded text was displayed on the monitor. The neural decoders in the system were bimodal in that they were jointly trained not only to synthesize speech but also to decode text simultaneously.

High-gamma activity (70-150 Hz) and low-frequency signals (0.3-17 Hz; see Methods) were streamed to a custom bimodal decoding model, which processed the neural features in 80 ms increments starting 500 ms before the go-cue in each trial to decode audible speech and text. Inspired by streaming automatic speech recognition (ASR) approaches [37-40], an RNN-transducer (RNN-T) framework [41] was used, a flexible, general-purpose neural network architecture initially designed for streaming ASR without needing future input context. This framework was adapted to facilitate streaming speech synthesis and text decoding from neural features. Specifically, a unidirectional recurrent neural network (RNN) processes neural features in real time, producing an encoding vector corresponding to the speech content. For speech synthesis, these encodings were combined autoregressively with a streaming discrete-speech unit language model to produce a probability distribution over the next discrete-speech unit from 100 candidates. Similarly, for text, the same encodings were combined autoregressively with a streaming sub-word text-encoding language model to produce a probability distribution over the next sub-word text-encoding from 4096 candidates. For discrete-speech units and text encodings, RNN-T beam search was used to determine the most likely token during inference [41]. The predicted discrete-speech unit was passed as input into a personalized speech synthesizer to generate a waveform chunk played synchronously as the participant attempted to speak (FIG. 38A; exemplar output waveform and spectrogram shown in FIG. 38B).

To overcome limitations in aligning neural data to speech behavior due to the lack of intelligible speech from the participant, the RNN-T loss function was employed during training. Notably, the RNN-T loss not only models the probability of the output discrete-speech units or text encodings but also models their interdependence. This allows for the learning of a streaming language model over the discrete-speech units and text encodings without requiring an external language model [41]. The streaming language model portions of the architecture were trained offline on speech-recognition tasks and were frozen before the rest of the pipeline was trained. The target discrete-speech units were extracted using HuBERT, a self-supervised speech-representation learning model that encodes a speech waveform into a temporal sequence of units that capture the speech waveform's underlying phonetic and articulatory characteristics [42,43]. Since the participant cannot speak, the initial speech waveforms were generated using a text-to-speech (TTS) model [44]. No audible vocalization was required to train the speech decoders. Finally, for the speech synthesizer, a personalized autoregressive discrete-speech unit synthesizer was trained, which models the duration of the discrete-speech units to better match the participant's speaking rate. The synthesized speech was conditioned on a short voice clip of the participant recorded before she lost her speech ability (see Methods).

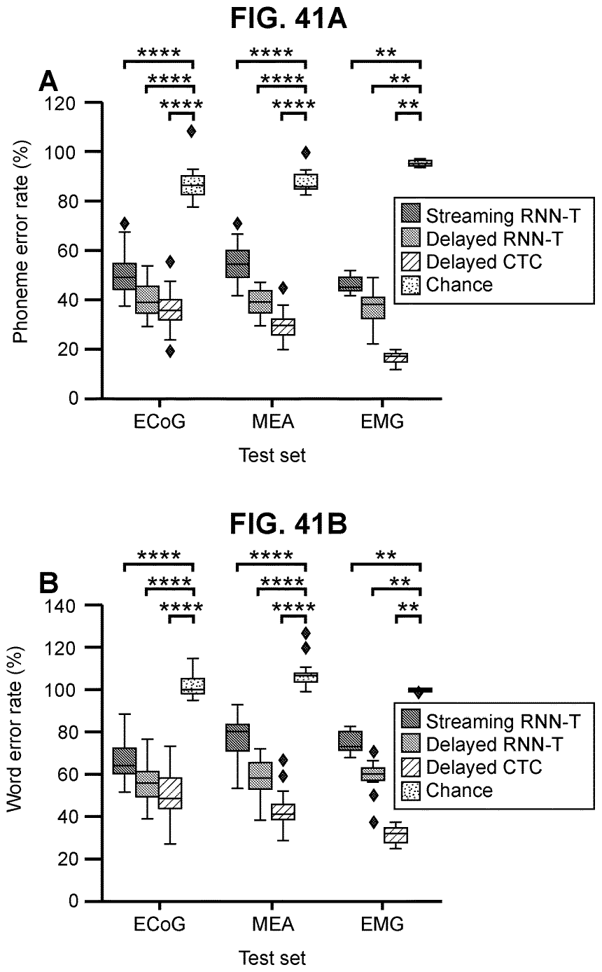

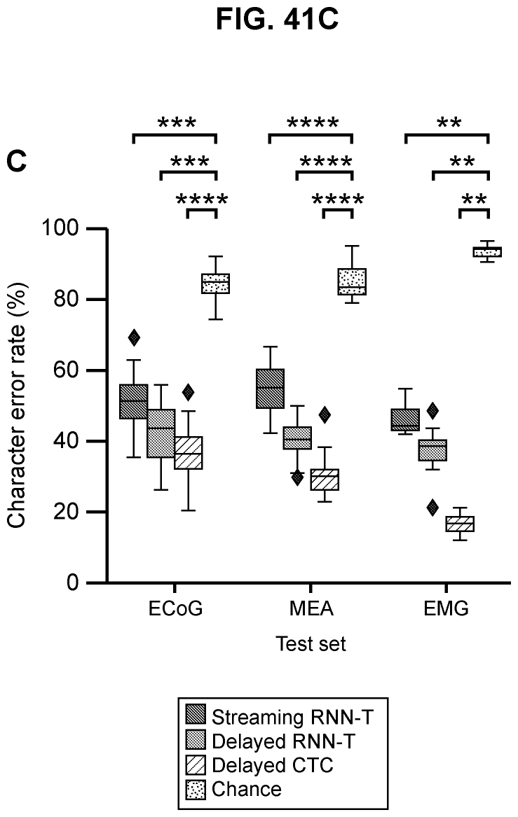

The system was evaluated using a small vocabulary sentence set “50-phrase-AAC” and an extensive vocabulary sentence set, “1024-word-General”. The 50-phrase-AAC set was designed as a predefined phrase set to express primary caregiving needs [45]. In contrast, the 1024-word-General set was designed as a large-vocabulary, large-sentence set containing 12,379 unique sentences composed of 1,024 unique words sampled from Twitter and movie transcriptions [22]. The participant completed nearly two full passes through the corpus during training, resulting in 23,378 total silent speech attempts. Each sentence was seen at least twice during training, and a subset of the sentences was also collected multiple times, resulting in the model seeing each test sentence an average of 6.94 times during training. Additionally, to test the generalizability of the neural decoder, performance was evaluated on novel sentences composed of words within the vocabulary but never seen by the participant (see FIG. 41).

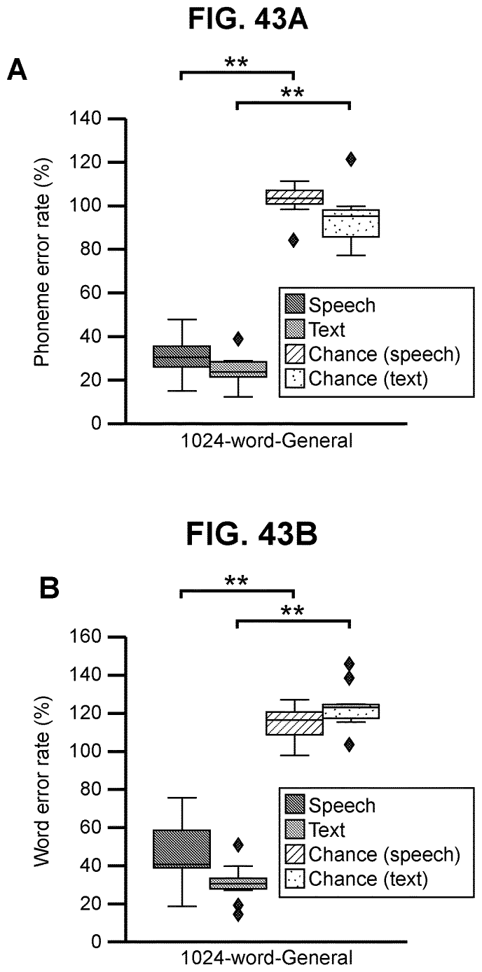

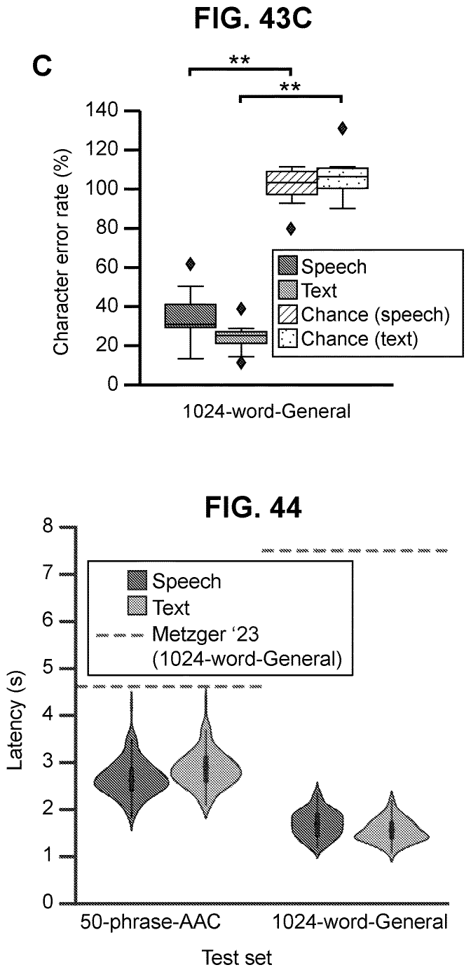

As an alternative to speech synthesis via discrete-speech units, a version of the pipeline was also implemented that performs text decoding followed by word-by-word TTS for the 1024-word-General sentence set. Here, the text-decoding portion of the same model was used to predict the next text segment, and this was then used to condition a TTS model that synthesized speech for that segment (Video 2; FIG. 43; Table 16). This offers higher intelligibility at the cost of fluency.

TABLE 16
Comparisons for real-time text-to-speech decoding. Each comparison
is a two-sided Wilcoxon signed-rank test between n =
10 pseudoblocks for the 1024-word-General set. 6-way Holm-
Bonferroni correction for multiple comparisons applied.

| | | Compari- | Compari- | | Corrected |
|---|---|---|---|---|---|
| Dataset | Metric | son 1 | son 2 | Statistic | P-value |
| 1024-word- | WER | Speech | Chance | 1.842e+04 | 1.172e−02 |
| General | | | (speech) | | |
| 1024-word- | WER | Text | Chance | 1.842e+04 | 1.172e−02 |
| General | | | (text) | | |
| 1024-word- | CER | Speech | Chance | 1.842e+04 | 1.172e−02 |
| General | | | (speech) | | |
| 1024-word- | CER | Text | Chance | 1.842e+04 | 1.172e−02 |
| General | | | (text) | | |
| 1024-word- | PER | Speech | Chance | 1.842e+04 | 1.172e−02 |
| General | | | (speech) | | |
| 1024-word- | PER | Text | Chance | 1.842e+04 | 1.172e−02 |
| General | | | (text) | | |

FIGS. 38A-38B: Overview of a naturalistic streaming silent-speech neuroprosthesis. FIG. 38A: Overview of the streaming speech-synthesis and text-decoding pipeline. A person with severe paralysis due to a brainstem stroke was implanted with a 253-channel electrocorticography array 18 years after injury. Deep-learning models were trained to map the neural activity during silently attempted speech to personalized speech and text in increments of 80 ms. For speech synthesis, discrete-speech units are decoded and then synthesized into speech. For text, sub-word text-encodings are predicted and then dequantized into words. For both outputs, a streaming language model takes in the previous prediction in parallel with neural encoder inference to allow for language modeling during streaming decoding. FIG. 38B: An exemplar online waveform (top) and spectrogram (bottom) from the 1024-word-General set. The detected go-cue and speech attempt are demarcated in black and green, respectively. Timings for text-emissions are demarcated in purple. All elements are temporally to scale.

FIG. 43: Real-time incremental text-to-speech decoding performance. Decoding accuracy for real-time text-to-speech decoding using the 1024-word-General sentence set. Instead of directly synthesizing speech, a text-to-speech model was used to incrementally synthesize the next word or sub-phrase in the predicted sentence. Chance distributions were generated by shuffling the electrode locations and applying the decoder. Predicted speech transcripts were obtained via the Whisper automatic speech recognition model. For speech, a median PER of 33.0% (99% CI [26.3, 51.5]) was observed. Median WER was 40.8% (99% CI [35.3, 62.7]), and median CER was 30.9% (99% CI [29.4, 50.0]). A median PER of 23.1% (99% CI [15.2, 28.5]) for text was observed. Median WER was 30.7% (99% CI [19.2, 40.0]), and median CER was 24.7% (99% CI [14.1, 28.0]). Performances were significantly better than chance (P<0.01 for all metrics, Wilcoxon signed-rank test with 6-way Holm-Bonferonni Correction for multiple comparisons). Statistics compare n=10 total pseudo-blocks (See Table 16).

### 2.3.2. Fast Streaming Intelligible Speech Synthesis and Text Decoding

To evaluate online performance, speech was synthesized and decoded text displayed as the participant silently attempted to say 100 different sentences from the 1024-word-General set (Video 1) and 150 trials (50 phrases, three times each) from the 50-phrase-AAC set.

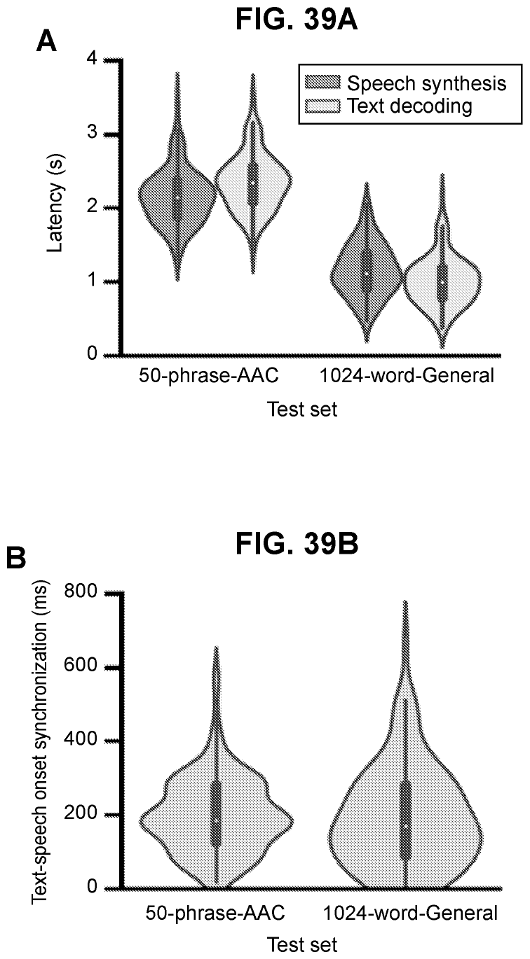

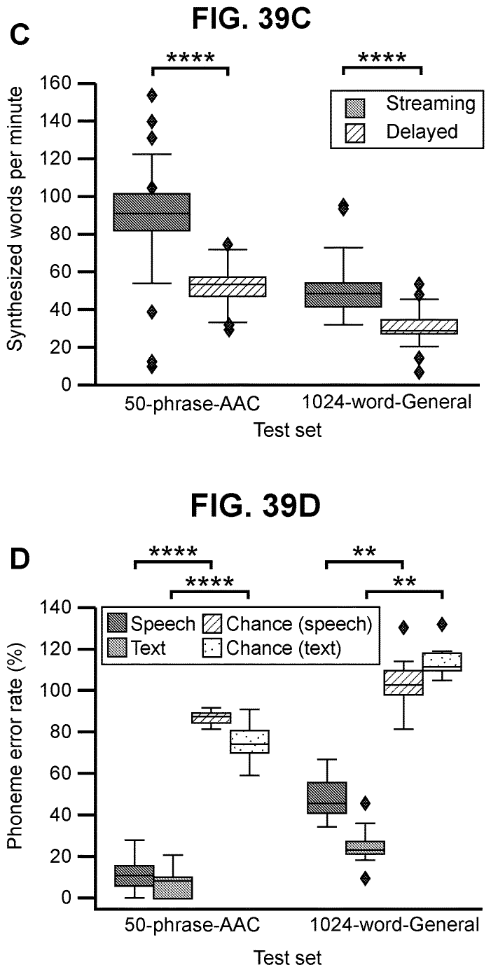

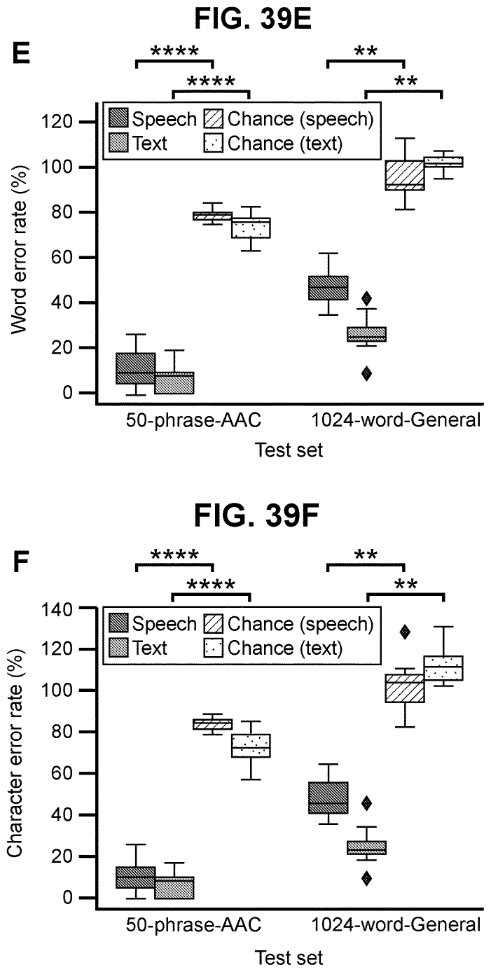

Minimal-delay speech synthesis and text decoding was observed as measured from the time between the speech attempt and the onset of decoding output. A speech detection algorithm was used to predict the start of the attempted speech from the participant's neural activity (see Methods) [22,24,25]. For the 1024-word-General set, median latencies of 1.12 s (99% CI [1.03, 1.26]) for speech synthesis and 1.01 s (99% CI [0.90, 1.13]) for text decoding were achieved, respectively. For the 50-phrase-AAC set, median latencies of 2.14 s (99% CI [2.05, 2.22]) for speech synthesis and 2.35 s (99% CI [2.28, 2.43]) for text decoding were achieved respectively (FIG. 39A). When measuring the latency between the go-cue and the decoding onsets, for the 1024-word-General set, median latencies of 1.67 s (99% CI [1.57, 1.81]) and 1.56 s (99% CI [1.4, 1.56]) for speech-synthesis and text-decoding were observed respectively, increased due to the participant's reaction time and the time between the beginning of articulation and intended acoustics. Similarly, for cue-based latency measurements, for the 50-phrase-AAC set, median latencies of 2.61 s (99% CI [2.53, 2.70]) and 2.90 s (99% CI [2.70, 2.90]) for speech-synthesis and text-decoding were achieved, respectively (FIG. 44). These latencies are faster than the participant's typical communication latency using her previous assistive communication device (23.2 seconds; Supplementary Note 2) and are lower latency than the previous state-of-the-art speech synthesizer (delayed synthesis) [22].

To better characterize the synchronization between the text and speech onsets, a median absolute timing difference between text onset and speech onset of 185 ms (99% CI [170, 210]) and 170 ms (99% CI [130, 210]) were observed for 1024-word-General and 50-phrase-AAC respectively (FIG. 39B). This is important because the text and the speech synthesis are jointly modeled, whereas all previous approaches model the outputs independently or are not multimodal at all and, hence, do not provide actual conditioning. Lastly, the system's decoding speed was observed to be a median 47.4 words per minute (WPM) (99% CI [41.0, 45.8]) and 90.5 WPM (99% CI [77.5, 85.1]) for the 1024-word-General and 50-phrase-AAC sets, respectively, (FIG. 39C) and was significantly faster than the previous decoding approach (P<0.0001 for both comparisons, two-sided Wilcoxon rank-sum tests, Table 17) [22]. The 50-phrase-AAC sentence set had a much higher median WPM because the participant's rate of silently attempted speech was, by design, faster for those sentences.

TABLE 17
Comparisons for decoding speed. Each comparison is a two-sided
Wilcoxon signed-rank test between n = 15 pseudo-blocks
for 50-phrase-AAC and n = 10 pseudo-blocks for 1024-word-General.

| | | Compari- | Compari- | | |
|---|---|---|---|---|---|
| Dataset | Metric | son 1 | son 2 | Statistic | P-value |
| 50-phrase- | WPM | Streaming | Metzger | 1.842e+04 | 1.023e−43 |
| AAC | | | ′23 | | |
| 1024-word- | WPM | Streaming | Metzger | 1.842e+-04 | 1.499e−32 |
| General | | | ′23 | | |

For the evaluation of the content of the decoded outputs, phoneme error rate (PER), word error rate (WER), and character error rate (CER), were used which are standard in automatic speech recognition and measure the percentage of incorrect phonemes, words, and characters, respectively. Error rates were computed across sequential pseudo-blocks of 10-sentence segments. For speech synthesis, transcripts were obtained from perceptual evaluations performed by third-party evaluators (see Methods; see Table 18 and Table 19 for example, 1024-word-General speech-synthesis and text-decoding transcripts, respectively). Median speech PERs of 10.8% (99% CI [4.21, 15.9]) and 45.3% (99% CI [40.7, 64.4]) for the 50-phrase-AAC and 1024-word-General sets were achieved, respectively. For text, PERs of 7.58% (99% CI [0.0, 9.27]) and 23.9% (99% CI [18.4, 31.6]) were achieved for the 50-phrase-AAC and 1024-word-General sets, respectively (FIG. 39E). Additionally, median speech WERs of 12.3% (99% CI [5.26, 23.1]) and 58.8% (99% CI [50.5, 76.0]) were achieved for the 50-phrase-AAC and 1024-word-General sets, respectively. For text, WERs of 10.3% (99% CI [0.0, 13.8]) and 31.9% (99% CI [27.5, 42.8]) for the 50-phrase-AAC and 1024-word-General sets were achieved, respectively (FIG. 39F). Similarly, median speech CERs of 11.2% (99% CI [4.67, 15.0]) and 44.7% (99% CI [39.2, 63.3]) for the 50-phrase-AAC and 1024-word-General sets were achieved, respectively. For text, CERs of 7.23% (99% CI [0.0, 9.72]) and 22.8% (99% CI [18.4, 31.0]) for the 50-phrase-AAC and 1024-word-General sets were achieved, respectively (FIG. 39G).

TABLE 18
Illustrative streaming speech-synthesis example transcripts for the 1024-word-
General set. Examples are shown for various levels of word error rate (WER) during
online decoding with the 1024-word-General set. Each percentile value indicates
the percent of transcripts from the synthesized sentences with a WER less than
or equal to the WER of the ground-truth sentence. Transcripts were obtained
from untrained evaluators transcribing what they heard (Method 2).

| Target sentence | Decoded sentence | WER (%) | Percentile (%) |
|---|---|---|---|
| What did you say to her | What did you say to her | 0 | 6 |
| Would you like that | Would you like that | 0 | 6 |
| You love me then | You love me then | 0 | 6 |
| Why did he tell you | Why did he tell you | 0 | 6 |
| What does she want | What does she want | 0 | 6 |
| Where did you get this | Where did you get that | 20 | 15 |
| I think you mean that | I think you worry that | 20 | 15 |
| I said come on | I can come on | 25 | 19 |
| You had to tell him | You why to make him | 40 | 29.5 |
| This is where i get off | This is where | 50 | 37.5 |
| What do you know about my father | What did you know that might | 57.1 | 42 |
| Do you know where [symbol] can find him | You will know he was him | 75 | 60.5 |
| Is there anything i can do | Is that they buy and chew | 83.3 | 71.5 |
| Like the last time | Is they that then | 100 | 85.5 |
| I just got here | I've said to stash it | 125 | 100 |

[symbol]

indicates data missing or illegible when filed

TABLE 19
Illustrative text-decoding examples. Examples are shown for various levels of word
error rate (WER) during real-time decoding with the 1024-word-General set. Each
percentile value indicates the percent of decoded sentences that had a WER less
than or equal to the WER of the provided example sentence. All examples are text-
decoding examples from real-time bimodal speech synthesis and text-decoding.

| Target sentence | Decoded text | WER (%) | WER Percentile (%) |
|---|---|---|---|
| If they like me | If they like me | 0.0 | 18.0 |
| How can we get out | How can we get out | 0.0 | 18.0 |
| Is that for me | Is that for me | 0.0 | 18.0 |
| What do you know about my father | What do you know about my father | 0.0 | 18.0 |
| What are you going to do with it | What are you going to do with it | 0.0 | 18.0 |
| You love me then | You love me then | 0.0 | 18.0 |
| What does that do | What does that do | 0.0 | 18.0 |
| Where did you get this | Where did you get this | 0.0 | 18.0 |
| What did he have | What did he have | 0.0 | 18.0 |
| How did you get in here | How did you get here | 16.7 | 39.0 |
| Why would you do that | What would you do that | 20.0 | 44.0 |
| I said come on | I can come on | 25.0 | 52.5 |
| You get much of that in here | You get money at that year | 57.1 | 71.0 |
| I can see that | I need these | 75.0 | 83.5 |
| But i found the money | Might as well | 100.0 | 96.5 |

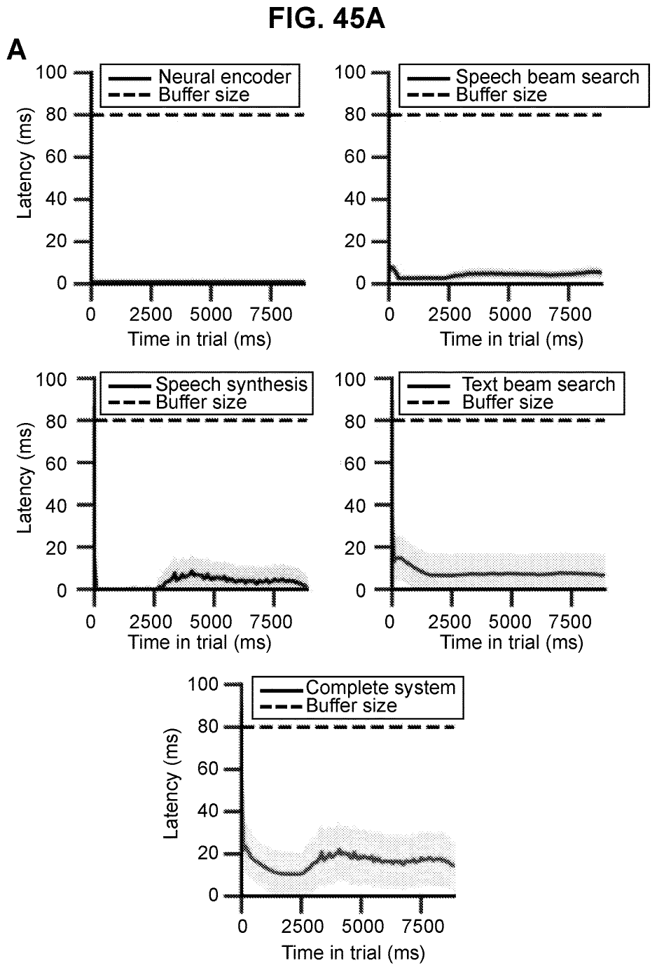

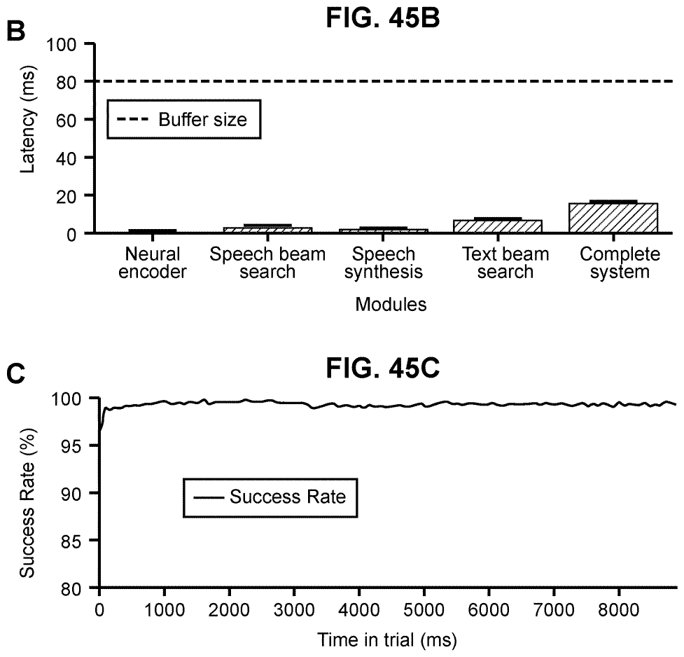

For all error rate metrics, performance was better than chance, which was computed by re-evaluating performance after shuffling the channels of the neural data and using this as the input to the decoding models (P<0.01 for all 12 comparisons, two-sided Wilcoxon rank-sum tests with 6-way Holm-Bonferroni correction, Table 20). Offline, the per-80-ms-chunk inference latency was characterized and over a 99% success rate was observed in achieving sub-80 ms latencies, with mean performances being 16.6 ms (99% CI [16.5, 16.6]) (FIG. 45). Together, these results demonstrate naturalistic streaming speech-synthesis from neural activity that produces low-latency intelligible speech.

TABLE 20
Comparisons for real-time bimodal decoding. Each comparison
is a two-sided Wilcoxon signed-rank test between n =
15 pseudo-blocks for 50-phrase-AAC and n = 10 pseudo-
blocks for 1024-word-General. 6-way Holm-Bonferroni correction
for multiple comparisons was used for each dataset.

| | | Compari- | Compari- | | Corrected |
|---|---|---|---|---|---|
| Dataset | Metric | son 1 | son 2 | Statistic | P-value |
| 50-phrase- | PER | Speech | Chance | 1.842e+04 | 3.662e−04 |
| AAC | | | (speech) | | |
| 50-phrase- | PER | Text | Chance | 1.842e+04 | 3.662e−04 |
| AAC | | | (text) | | |
| 50-phrase- | WER | Speech | Chance | 1.842e+04 | 3.662e−04 |
| AAC | | | (speech) | | |
| 50-phrase- | WER | Text | Chance | 1.842e+04 | 3.662e−04 |
| AAC | | | (text) | | |
| 50-phrase- | CER | Speech | Chance | 1.842e+04 | 3.662e−04 |
| AAC | | | (speech) | | |
| 50-pbrase- | CER | Text | Chance | 1.842e+04 | 3.662e−04 |
| AAC | | | (text) | | |
| 1024-word- | PER | Speech | Chance | 1.842e+04 | 1.172e−02 |
| General | | | (speech) | | |
| 1024-word- | PER | Text | Chance | 1.842e+04 | 1.172e−02 |
| General | | | (text) | | |
| 1024-word- | WER | Sprech | Chance | 1.842e+04 | 1.172e−02 |
| General | | | (speech) | | |
| 1024-word- | WER | Text | Chance | 1.842e+04 | 1.172e−02 |
| General | | | (text) | | |
| 1024-word- | CER | Speech | Chance | 1.842e+04 | 1.172e−02 |
| General | | | (speech) | | |
| 1024-word- | CER | Text | Chance | 1.842e+04 | 1.172e−02 |
| General | | | (text) | | |

FIGS. 39A-39F: Online continuously streaming synchronized speech synthesis and text decoding from neural activity. FIG. 39A: Latency for speech-synthesis and text-decoding. Latency is defined as the time from the detected start of attempted speech to decoding output onset. FIG. 39B: Synchronization time between the speech-synthesis onset and text-decoding onset. FIG. 39C: Synthesized words per minute compared to delayed synthesis [22]. FIG. 39D: Phoneme error rates. FIG. 39E: Word error rates. FIG. 39F: Character error rates. FIGS. 39D-39F: Decoded speech-synthesis transcripts were obtained from untrained human evaluators via a transcription task, n=15 and n=10 for the 50-phrase-AAC set 1024-word-General set, respectively. FIGS. 39C-39F: ****P<0.0001, **P<0.001, Two-sided Wilcoxon Signed-Rank test with 6-way Holm-Bonferroni correction for multiple comparisons; P-values and statistics in Table 20. Box plots in all figures depict the median (horizontal line inside box), 25th and 75th percentiles (box), 25th and 75th percentiles+/−1.5 times the interquartile range (whiskers), and outliers (diamonds). FIGS. 39A-39F: All results are obtained online. Performances for online text-to-speech synthesis can be found in FIG. 43.

FIG. 44: Go-cue to speech-decoding latency. Full distribution of latencies calculated by taking the time difference between the speech or text onset and the go cue. For the 1024-word-General set, median latencies of 1.67 s (99% CI [1.57, 1.81]) for speech synthesis and 1.56 s (99% CI [1.4, 1.56]) for text decoding were achieved, respectively. For the 50-phrase-AAC set, median latencies of 2.61 s (99% CI [2.53, 2.7]) for speech synthesis and 2.90 s (99% CI [2.7, 2.9]) for text decoding were achieved, respectively. The dashed line denotes previous state-of-the-art speech-synthesis BCI latency in a person with paralysis.

FIGS. 45A-45C: Characterization of inference latency. Characterization of the latency of the speech synthesis and text decoding system. Offline, the decoder was re-applied to the real-time test trials (30 re-applications for each trial). The latency of the inference procedure was measured at each time step. FIG. 45A: Latency per time step for the neural encoder, speech joiner and beam search, speech synthesizer, text joiner and beam search, and complete system. Each module is reported in a separate panel. FIG. 45B: Latency by module; Neural encoder: 1.00 ms (99% CI [1.00, 1.01]), Speech beam search 4.29 ms (99% CI [4.28, 4.30]), Speech synthesizer: 3.21 ms (99% CI [3.18, 3.23]), Text joiner beam search: 8.07 ms (99% CI [8.03, 8.11]), Complete system: 16.6 ms (99% CI [16.5, 16.6]). The confidence interval (CI) is measured by random bootstrapping around mean latency. The beam search subsumed the inference time of the joiner and predictor. The text beam search had the longest latency among the modules. However, the total latency, on average, was far smaller than the buffer size of 80 ms, indicating that inference speed is relatively low-latency. FIG. 45C: Success rate averaged across time for all trials. Measured by the ratio of complete-system inference below 80 ms latency, observed a success rate of 99.3% (99% CI [97.60, 99.73]).

2.3.3. Long-Form Continuous Speech Decoding with Implicit Speech Detection

Ideal speech neuroprostheses should be able to operate continuously. They should not be confined to isolated trial-level attempts, enabling future speech-BCI users to use the device outside a dedicated task structure. Although online continuous speech synthesis at the trial level was demonstrated, the speech synthesizer should ideally generalize to longer-form decoding (i.e., over several minutes or hours rather than several seconds). Furthermore, previous approaches to speech detection operated using a separate model, which resulted in waiting until the end of the speech attempt to decode speech [22,24-26], hence not demonstrating continuous low-latency speech decoding. Ideal speech neuroprostheses should be able to operate continuously using a unified model that is aware of when the participant is speaking or not. To address this offline, the 1024-word-General sentence-set model was applied to 4 entire blocks of neural activity consisting of 25 trials each. Specifically, instead of passing in neural data 500 ms before the trial and waiting for a fixed period to allow for trial-level decoding, the entire block of neural activity was autoregressively passed to the model in non-overlapping chunks of 80 ms.

It was observed that the transducer model offers an implicit speech-detection mechanism that allows it to operate continuously over periods beyond single trials. Once the transducer is confident enough that speech is occurring, it begins synthesizing speech. Likewise, it rarely produces speech when speech is not implicitly detected (see Methods, FIG. 40A). Only three false-positive speech-synthesis segments were observed out of 100 in which the participant had not attempted speech. This demonstrates that the system correlates with volitional silent speech attempts and rarely produces speech when not desired. Additionally, no generated speech was observed when applying the model to 16 minutes of rest data (during which the participant was instructed to rest and do nothing) aggregated across ten sessions. To characterize this further, the latency (absolute difference) between the speech-synthesizer outputs and the detected onsets and offset for silently attempted speech was computed (i.e., using a separate speech detection model; see Methods). Median latencies of 1.19 s (99% CI [1.09, 1.29]) for onsets and 1.89 s (99% CI [1.70, 2.04]) for offsets were observed (FIG. 40B).

Error rates were also computed between the predicted and target speech and text during long-form decoding. For speech, median PERs of 49.4% (99% CI [44.5, 56.7]), median WERs of 65.0% (99% CI [60.3, 73.1]), and median CERs of 49.3% (99% CI [43.9, 55.2]) were achieved. For text, median PERs of 34.7% (99% CI [26.5, 40.7]), median WERs of 44.2% (99% CI [33.1, 51.5]), and median CERs of 34.1% (99% CI [25.9, 39.2]) were achieved (FIG. 40C-40E). All performances were above chance (P<0.0001 for all 6 comparisons, two-sided Wilcoxon rank-sum tests with 6-way Holm-Bonferroni correction computed over median-bootstrapped block metrics with 1000 iterations sampled with replacement; Table 21). These results demonstrate continuous speech synthesis beyond isolated trial-level attempts and offer promise for the future development of clinical user interfaces for continuous speech decoding.

TABLE 21
Comparisons for Long-form speech-synthesis and text-
decoding. Each comparison is a two-sided Wilcoxon signed-
rank test between n = 1000 bootstrapped median
metrics for the 1024-word General set. 3-way Holm-Bonferroni
correction for multiple comparisons applied.

| | | Compari- | Compari- | | Corrected |
|---|---|---|---|---|---|
| Dataset | Metric | son 1 | son 2 | Statistic | P-value |
| 1024-word- | PER | Speech | Chance | 0.000e+00 | 1.156e−164 |
| General | | | | | |
| 1024-word- | PER | Text | Chance | 0.000e+00 | 1.156e−164 |
| General | | | | | |
| 1024-word- | WER | Speech | Chance | 0.000e+00 | 1.156e−164 |
| General | | | | | |
| 1024-word- | WER | Text | Chance | 0.000e+00 | 1.156e−164 |
| General | | | | | |
| 1024-word- | CER | Speech | Chance | 0.000e+00 | 1.156e−164 |
| General | | | | | |
| 1024-word- | CER | Text | Chance | 0.000e+00 | 1.156e−164 |
| General | | | | | |

FIGS. 40A-40E: Offline long-form continuous speech decoding with implicit speech detection. FIG. 40A: Top: a heatmap of log-scaled high-gamma activity (HGA) from the top 20 most speech-responsive electrodes during silent speech attempts of an entire block (5.9 minutes) of 1024-word-General sentences. Lighter indicates increased neural activity. Bottom: a continuously synthesized speech waveform from the aforementioned neural activity. Neural data is passed into the model in 80 ms chunks and synthesized continuously in 80 ms chunks. The go-cue detected silent speech-attempt onsets and detected silent speech-attempt offsets are marked in black, green, and purple, respectively. Gray indicates single trials. Specifically, the decoder had access to the original time region of neural data during online inference. FIG. 40B: Latency between the detected onset of silently attempted speech to synthesized speech output onset and latency between the detected offset of silently attempted speech to synthesized speech output offset. Each point in the distribution is a single trial. FIG. 40C: Phoneme error rates. FIG. 40D: Word error rates. FIG. 40E: Character error rates. FIGS. 40C-40E: Predicted speech transcripts were obtained via automatic speech recognition. Chance is computed by shuffling the electrodes and applying the decoder. Each dot indicates performance computed via continuous speech synthesis and text decoding over an entire block of attempted speech.

### 2.3.4. Generalization Across Silent-Speech Interfaces

Provided that the spatiotemporal resolution and coverage of the neural recording device are sufficient, decoding approaches for speech-neuroprostheses should be able to generalize beyond a single recording modality or participant [46]. Furthermore, any such silent-speech recording interface with an articulatory basis should be able to be used as a synthesizer for intelligible speech [32]. To this end, the speech-decoding approach was applied to three separate silent-speech datasets: A previous 1024-word-General ECoG corpus from the participant [22], an open-vocabulary microelectrode array (MEA) dataset from a person with paralysis implanted with an intracortical BCI [23], and an articulatory electromyography dataset (EMG) from a single healthy speaker with surface electrodes placed along their vocal tract [35]. The EMG dataset was included as a baseline for silent-speech synthesis, where healthy speakers are able to make the full range of articulatory movements. Note that although each recording modality is a proxy for articulatory behavior during silent speech attempts, the evaluation sentences, device implant, amount of training data, participant behavior, and participant etiologies differ. Hence, this analysis helps us to understand the generalization of the proposed sequence modeling framework but, for the stated reasons, the performances of the three recording interfaces were not directly compared.

For each of the three datasets, data recorded during silent-speech attempts was tested on, no vocalized audio from the participants during the training of the models was used, and unseen sentences were tested on. For MEA and EMG, the speech-synthesizer voice was not personalized. Recording-modality-specific feature-extraction layers were used before applying the recurrent neural network in the encoder (see Methods). Otherwise, the architecture was the same as demonstrated for online decoding (FIG. 39A). For ECoG, a median PER of 49.0% (99% CI [44.2, 57.9]), a median WER of 63.8% (99% CI [57.5, 73.9]), and a median CER of 51.3% (99% CI [46.1, 58.9]) were observed. For MEA, a median PER of 54.5% (99% CI [48.3, 60.9]), a median WER of 79.7% (99% CI [70.5, 84.0]), and a median CER of 55.0% (99% CI [48.6, 61.7]) were observed. For EMG, a median PER of 44.8% (99% CI [43.3, 49.9]), a median WER of 73.0% (99% CI [69.0, 80.9]), and a median CER of 44.3% (99% CI [42.5, 49.9]) were observed.

To assess the effects of using the entire window of neural activity on performance, the speech-synthesis models was retrained but a streaming constraint was not enforced. No 80-ms streaming buffer was used for all three models, allowing the model to produce delayed synthesis at the expense of latency. For ECoG and MEA, bidirectional neural encoders were used. A unidirectional neural encoder was used for EMG. For ECoG, a median PER of 38.7% (99% CI [34.4, 46.2]), a median WER of 55.4% (99% CI [49.1, 60.8]), and a median CER of 43.7% (99% CI [35.6, 49.0]) were observed. For MEA, a median PER of 39.1% (99% CI [31.9, 44.9]), a median WER of 57.9% (99% CI [50.0, 66.7]), and a median CER of 40.8% (99% CI [33.2, 44.2]) were observed. For EMG, a median PER of 37.9% (99% CI [31.4, 42.0]), a median WER of 59.7% (99% CI [53.3, 64.2]), and a median CER of 38.5% (99% CI [33.1, 42.0]) were observed.

A connectionist temporal classification (CTC)-based approach (described in [22]) was also applied, which requires a full window of neural activity to each modality-achieving intelligible but delayed speech synthesis. For ECoG, a median PER of 35.7% (99% CI [31.9, 40.8]), a median WER of 48.4% (99% CI [41.2, 58.7]), and a median CER of 36.5% (99% CI [31.6, 41.1]) were observed. For MEA, a median PER of 29.9% (99% CI [22.2, 32.4]), a median WER of 40.9% (99% CI [38.3, 46.6]), and a median CER of 30.1% (99% CI [24.0, 32.9]) were observed. For EMG, a median PER of 17.5% (99% CI [14.1, 20.4]), a median WER of 32.0% (99% CI [26.7, 34.9]), and a median CER of 16.8% (99% CI [13.9, 21.3]) were observed (FIG. 41A-41C).

Significantly above-chance performance was observed for each modality and error rate metric (P<0.01 for all 27 comparisons, two-sided Wilcoxon rank-sum tests with 9-way Holm-Bonferroni correction computed over median-bootstrapped block metrics with 1000 iterations sampled with replacement; Table 22). Together, these results show that the streaming and delayed model architectures generalize across silent-speech interfaces and are not limited to ECoG. Error rate metrics decrease with increased delay, at the expense of streaming, speed, and latency. The performance of these approaches is expected to improve and further generalize as the density or number of electrodes increases when recording from the speech sensorimotor cortex.

TABLE 22
Comparisons for modality generalization. Each comparison is a two-sided
Wilcoxon signed-rank test between n = 20 pseudo-blocks for ECoG,
n = 12 pseudo-blocks for MEA, and n = 10 pseudo-blocks for EMG.
9-way Holm-Bonferroni correction for multiple comparisons was used for each dataset.

| Dataset | Metric | Comparison 1 | Comparison 2 | Statistic | Corrected P-value |
|---|---|---|---|---|---|
| ECoG | PER | Streaming RNN-T | Chance | 0.000e+00 | 1.717e−05 |
| ECoG | PER | Full-context RNN-T | Chance | 0.000e+00 | 1.717e−05 |
| ECoG | PER | Full-context CTC | Chance | 0.000e+00 | 1.717e−05 |
| ECoG | WER | Streaming RNN-T | Chance | 0.000e+00 | 1.717e−05 |
| ECoG | WER | Full-context RNN-T | Chance | 0.000e+00 | 1.717e−05 |
| ECoG | WER | Full-context CTC | Chance | 0.000e+00 | 1.717e−05 |
| ECoG | CER | Streaming RNN-T | Chance | 0.000e+00 | 1.171e−03 |
| ECoG | CER | Full-context RNN-T | Chance | 0.000e+00 | 1.171e−03 |
| ECoG | CER | Full-context CTC | Chance | 0.000e+00 | 1.717e−05 |
| MEA | PER | Streaming RNN-T | Chance | 0.000e+00 | 2.747e−04 |
| MEA | PER | Full-context RNN-T | Chance | 0.000e+00 | 2.747e−04 |
| MEA | PER | Full-context CTC | Chance | 0.000e+00 | 2.747e−04 |
| MEA | WER | Streaming RNN-T | Chance | 0.000e+00 | 2.747e−04 |
| MEA | WER | Full-context RNN-T | Chance | 0.000e+00 | 2.747e−04 |
| MEA | WER | Full-context CTC | Chance | 0.000e+00 | 2.747e−04 |
| MEA | CER | Streaming RNN-T | Chance | 0.000e+00 | 2.747e−04 |
| MEA | CER | Full-context RNN-T | Chance | 0.000e+00 | 2.747e−04 |
| MEA | CER | Full-context CTC | Chance | 0.000e+00 | 2.747e−04 |
| EMG | PER | Streaming RNN-T | Chance | 0.000e+00 | 1.758e−02 |
| EMG | PER | Full-context RNN-T | Chance | 0.000e+00 | 1.758e−02 |
| EMG | PER | Full-context CTC | Chance | 0.000e+00 | 1.758e−02 |
| EMG | WER | Streaming RNN-T | Chance | 0.000e+00 | 1.758e−02 |
| EMG | WER | Full-context RNN-T | Chance | 0.000e+00 | 1.758e−02 |
| EMO | WER | Full-context CTC | Chance | 0.000e+00 | 1.758e−02 |
| EMG | CER | Streaming RNN-T | Chance | 0.000e+00 | 1.758e−02 |
| EMG | CER | Full-context RNN-T | Chance | 0.000e+00 | 1.758e−02 |
| EMO | CER | Full-context CTC | Chance | 0.000e+00 | 1.758e−02 |

FIGS. 41A-41C: Speech synthesis generalization across silent-speech interfaces. FIG. 41A: Phoneme error rates. FIG. 41B: Word error rates. FIG. 41C: Character error rates. FIGS. 41A-41C: To evaluate the generalizability of the approach, a streaming RNN-T model, an RNN-T model without a streaming buffer constraint (delayed), and a CTC model (also delayed) were retrained and tested. Predicted speech transcripts were obtained via automatic speech recognition. Chance distributions were generated by shuffling the electrode locations and applying the best-performing decoder. n=20, n=12, and n=10 pseudo-blocks for electrocorticography (ECoG), microelectrode arrays (MEA), and surface electromyography (EMG), respectively. **P<0.01, ***P<0.001, ****P<0.0001, Two-sided Wilcoxon Signed-Rank test with 9-way Holm-Bonferroni correction for multiple comparisons; P-values and statistics in Table 22). Box plots in all figures depict the median (horizontal line inside box), 25th and 75th percentiles (box), 25th and 75th percentiles+/−1.5 times the interquartile range (whiskers), and outliers (diamonds). All models were tested offline.

2.3.5. Model-Generated Auditory Feedback does not Interfere with Articulatory-Driven Speech Decoding

As the decoder was trained without auditory feedback, one could hypothesize that, during inference where auditory feedback is present, neural-activity patterns could differ, especially in areas responsive to listening [49-51], which could disrupt the model's inference. In order to assess this, the electrode contributions and decoding performance on trials with and without naturalistic auditory feedback using synthesized speech were compared. The prompted texts were constrained to be consistent across conditions, leaving 50 trials for each condition. Both conditions used the 1024-word-General sentence set.

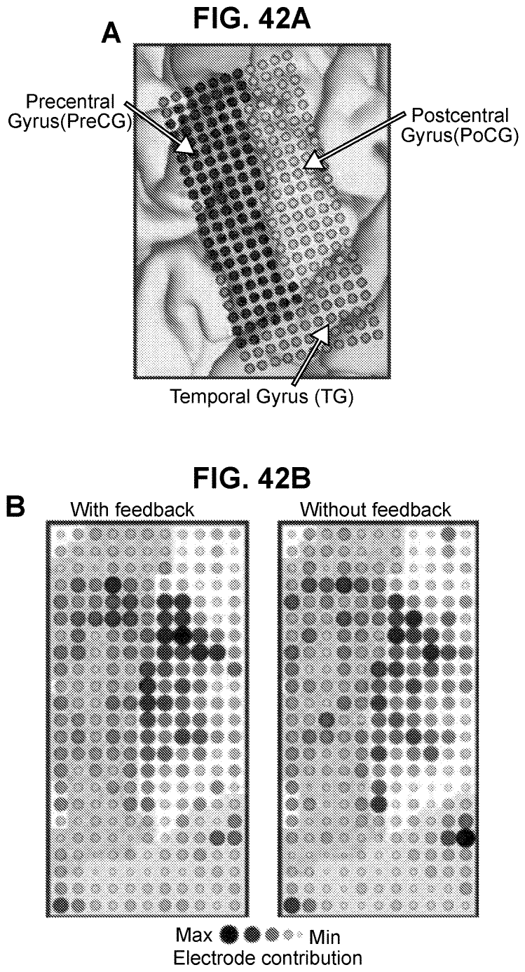

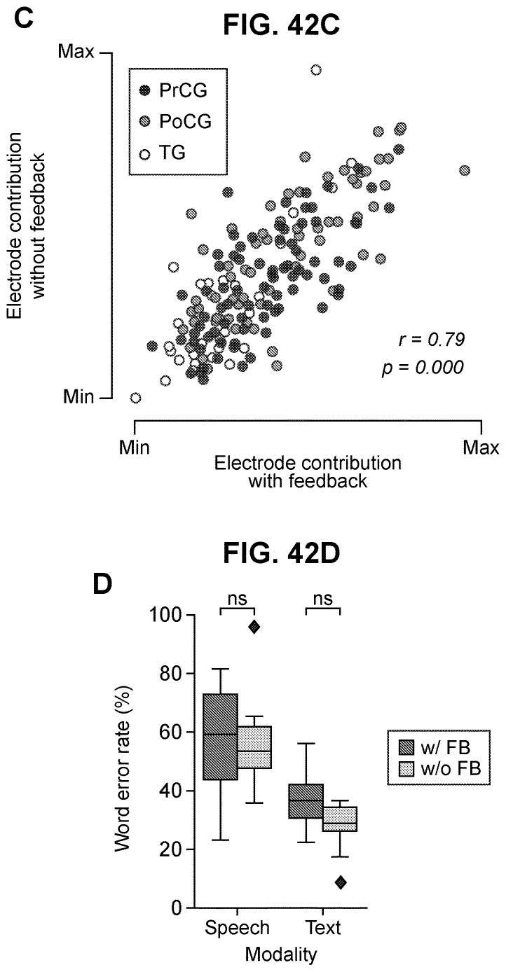

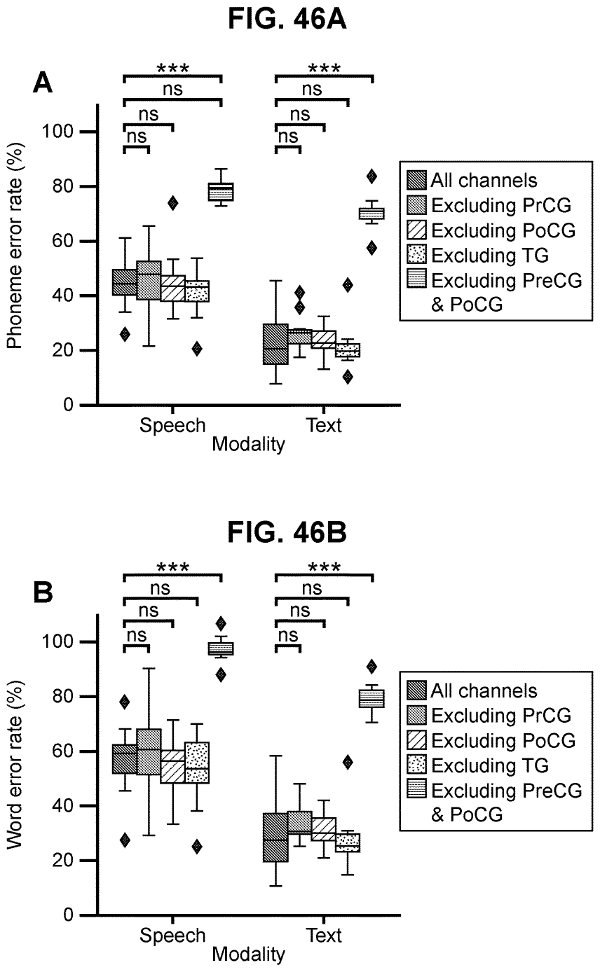

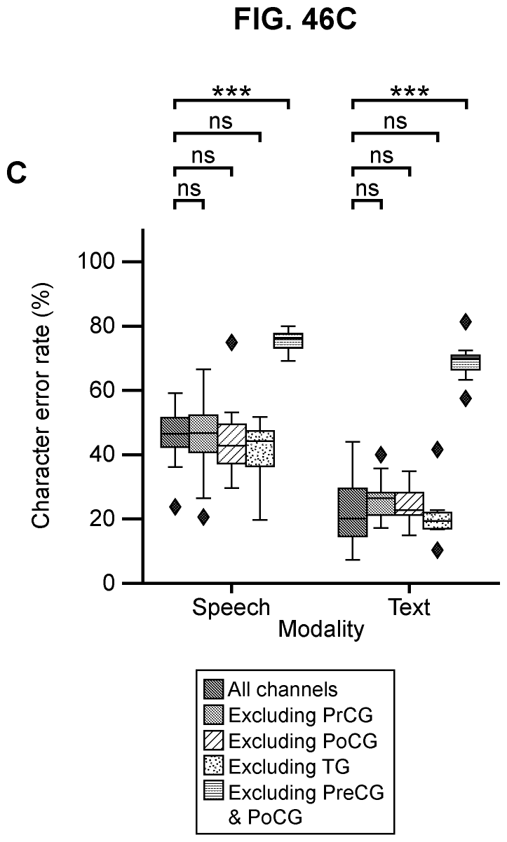

An ablation-based salience mapping technique was applied to calculate the amount of the contribution of each electrode on the array to streaming speech decoding (see Methods). The strongest contributions came from electrodes along the central sulcus and a middle section of the precentral gyrus (middle PreCG). Most of the electrodes on the superior temporal gyrus (STG) had relatively low contributions, except for two smaller clusters on the anterior and dorsal-posterior portions of the array's STG coverage (FIG. 42A-42B). The spatial patterns of electrode contributions are highly consistent regardless of the presence of feedback, with a significant and high correlation of 0.79 (Pearson r; P=0.000; Pearson correlation permutation test) (FIG. 42C). In terms of decoding performance, there was no significant difference between the two conditions for both modalities (FIG. 42D). In both modalities, the median WERs were lower without auditory feedback, however, the difference was insignificant (P=0.95 for speech and P=0.09 for text using a Two-sided Wilcoxon Signed-Rank test corrected by 2-way Holm-Bonferroni correction). Furthermore, region exclusion analysis was performed for the auditory feedback condition for the PoCG, PreCG, and STG, similar performance trends to those reported in prior work with this participant without auditory feedback (FIG. 46; Tables 23 and 24) [22]. In summary, it was shown that the decoders were unaffected by model-generated auditory feedback, instead relying on electrodes that correspond to articulatory control.

TABLE 23
Decoding accuracy by the length of training data. Median
error rates (%) and confidence intervals in FIG. 46.

| | | | Training data | | |
|---|---|---|---|---|---|
| Dataset | Metric | Modality | [symbol] (weak range) | Median | 99% CI |
| 1024-word-General | PER | Speech | Excluding PrCG | 48.02 | [31.74, 53.72] |
| 1024-word-General | PER | Speech | Excluding PoCG | 4 [symbol] .55 | [35.50, 50.67] |
| 1024-word-General | PER | Speech | Excluding TG | 43.45 | [31.9 [symbol] , 50.29] |
| 1024-word-General | PER | Speech | Excluding PreCG & PoCG | 79.43 | [74.66, 82.01] |
| 1024-word-General | PER | Text | Excluding PrCG | 26. [symbol] | [20.32, [symbol] 1.77] |
| 1024-word-General | PER | Text | Excluding PoCG | 22.6 [symbol] | [20.85, 29.14] |
| 1024-word-General | PER | Text | Excluding TG | 19.7 [symbol] | [17.03, 24. [symbol] ] |
| 1024-word-General | PER | Text | Excluding PreCG & PoCG | 7 [symbol] .32 | [66.47, 74.85] |
| 1024-word-General | WER | Speech | Excluding PrCG | 60. [symbol] | [43.27, 69.54] |
| 1024-word-General | WER | Speech | Excluding PoCG | 5 [symbol] .43 | [43.56, 62.39] |
| 1024-word-General | WER | Speech | Excluding TG | 5 [symbol] .59 | [38. [symbol] 3, 64.08] |
| 1024-word-General | WER | Speech | Excluding PreCG & PoCG | 96.12 | [94.78, 101. [symbol] ] |
| 1024-word-General | WER | Text | Excluding PrCG | [symbol] 0.78 | [28.04, 42.40] |
| 1024-word-General | WER | Text | Excluding PoCG | 29.63 | [26.79, 38.00] |
| 1024-word-General | WER | Text | Excluding TG | 24.97 | [19.61, 30.38] |
| 1024-word-General | WER | Text | Excluding PreCG & PoCG | 78.68 | [75. [symbol] 0, 84.09] |
| 1024-word-General | CER | Speech | Excluding PrCG | [symbol] .72 | [33.51, 53.90] |
| 1024-word-General | CER | Speech | Excluding PoCG | 42. [symbol] | [35.0 [symbol] , 51.53] |
| 1024-word-General | CER | Speech | Excluding TG | 44.16 | [31.65, [symbol] 9.28] |
| 1024-word-General | CER | Speech | Excluding PreCG & PoCG | 76. [symbol] 0 | [72.10, 79.66] |
| 1024-word-General | CER | Text | Excluding PrCG | 26.22 | [2 [symbol] .28, 32.21] |
| 1024-word-General | CER | Text | Excluding PoCG | 22.63 | [20.1 [symbol] , 30.53] |
| 1024-word-General | CER | Text | Excluding TG | 19.1 [symbol] | [16.84, 22.49] |
| 1024-word-General | CER | Text | Excluding PreCG & PoCG | 69.98 | [64.60, 72.46] |

[symbol]

indicates data missing or illegible when filed

TABLE 24
Comparisons of different training data length.

| [symbol] | [symbol] | [symbol] | [symbol] | [symbol] | [symbol] | [symbol] |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Each comparison is a Two-sided Wilcoxon Signed-Rank test between n = 10 pseudo-blocks for 1024-word-General. 4-way Holm-Bonferroni correction for multiple comparisons was applied within metrics and modalities. [symbol]
indicates data missing or illegible when filed

FIGS. 42A-42D: Model-generated auditory feedback does not interfere with articulatory-driven speech decoding. FIG. 42A: Placement of the electrodes on the speech sensorimotor cortex: precentral gyrus (PreCG; blue), postcentral gyrus (PoCG; red), and temporal gyrus (TG; green). FIG. 42B: Contribution maps calculated from two conditions: blocks with auditory feedback during online speech-synthesis demonstrations (left) and blocks without decoder feedback (right). Both conditions use the 1024-word-General sentence set. The values are normalized to be in [0,1], which is done separately for each condition. The contribution is calculated for each electrode by computing the difference in RNN-T loss induced by ablating the electrode. Both conditions show similar across-channel patterns of contributions. FIG. 42C: Contribution comparison for each channel, colored by anatomical region: PrCG (blue), PoCG (red), and TG (green). The correlation between electrode contributions from the two conditions is 0.79 (Pearson's correlation r=0.79 with P=0.000 (by region, PrCG: r=0.74, P=0.000; PoCG: r=0.81, P=0.000; TG: r=0.80, P=0.000); Pearson correlation permutation test). FIG. 42D: For both speech and text, there is no significant difference in decoding performance between conditions (w/FB=with feedback, w/o FB=without feedback; for both modalities, P=0.95 for speech, P=0.09 for text measured by Two-sided Wilcoxon Signed-Rank test with 2-way Holm-Bonferroni correction; n=10 pseudo-blocks for each modality and condition; ns P>0.05; Tables 25 and 26).

TABLE 25
Decoding performance of models with and without auditory feedback. Median error
rates (%) and confidence intervals with and without feedback, expanding FIG.
41E in the main text, measured in three forms: phoneme (PER), word (WER), and
character (CER). ASR is applied to get the text script from the speech.

| Dataset | Metric | Modality | Feedback | Median | 99% CI |
|---|---|---|---|---|---|
| 1024-word-General | PER | Speech | Auditory feedback | 42.22 | [30.20, 59.34] |
| 1024-word-General | PER | Speech | No feedback | 40.16 | [36.11, 49.91] |
| 1024-word-General | PER | Text | Anditory feedback | 26.88 | [18.60, 35.62] |
| 1024-word-General | PER | Text | No feedback | 24.23 | [17.22, 27.59] |
| 1024-word-General | WER | Speech | Auditory feedback | 59.10 | [41.29, 77.27] |
| 1024-word-General | WER | Speech | No feedback | 53.20 | [43.48, 65.22] |
| 1024-word-General | WER | Text | Auditory feedback | 36.67 | [26.92, 42.89] |
| 1024-word-General | WER | Text | No feedback | 29.00 | [21.66, 36.36] |
| 1024-word-General | CER | Speech | Auditory feedback | 43.89 | [28.41, 56.64] |
| 1024-word-General | CER | Speech | No feedback | 40.15 | [33.58, 46.57] |
| 1024-word-General | CER | Text | Auditory feedback | 26.37 | [19.05, 32.14] |
| 1024-word-General | CER | Text | No feedback | 21.61 | [17.18, 27.47] |

TABLE 26
Comparisons for with and without auditory feedback.

| [symbol] | [symbol] | [symbol] | [symbol] | [symbol] | [symbol] | [symbol] |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Each comparison is a Two-sided Wilcoxon Signed-Rank test between n = 10 pseudo-blocks with 5 trials for 1024-word-General. 2-way Holm-Bonferroni correction for multiple comparisons was applied within metrics. [symbol]
indicates data missing or illegible when filed

FIGS. 46A-46C: Region-exclusion analysis for 1024-word-General decoding. Decoding accuracy is compared across different channel configurations (all channels, excluding precentral gyrus (PrCG), postcentral gyrus (PoCG), temporal gyrus (TG), or motor (PrCG & PoCG)). FIG. 46A: Phoneme error rates. FIG. 46B: Word error rates. FIG. 46C: Character error rates. FIGS. 46A-46C: For speech, ASR on the synthesized speech was used for measuring the error rates. In all metrics, no significant difference was observed for ablating PrCG, PoCG, or TG (* * *P<0.001, ns >0.05, Two-tailed Wilcoxon signed-rank test with 6-way Holm-Bonferonni Correction for multiple comparisons). However, the accuracies significantly dropped by ablating entire motor regions (both pre- and post-CG). Summary statistics in Tables 23 and 24.

### 2.4. SUMMARY AND CONCLUSIONS

Naturalistic continuous speech synthesis from neural activity with minimal delay is a significant goal for technologies that restore speech to persons with severe paralysis [1,2,5,6,52]. It was demonstrated that this can be achieved using high-density surface recordings of cortical activity in the speech sensorimotor cortex. The deep-learning models of the disclosure could stream synthesized speech in just 80-ms increments and with simultaneous text decoding, using the participant's neural activity as she silently attempted to speak complete sentences from a 1,024-word vocabulary. Speech-evoked neural activity was time-aligned with decoded outputs, suggesting a learned alignment between brain data and target speech.

Previous demonstrations of speech synthesis from cortical activity have primarily been conducted with participants who could speak and did not need assistive communication technology for speech [18,19,21,53]. Recent demonstrations for restoring lost speech suffer from high latency or only predict text [17,24-26]. Improving speech-synthesis latency and decoding speed is essential for dynamic conversation and fluent communication, which is compounded by the fact that speech synthesis requires additional time to play and for the user and listener to comprehend the synthesized audio. In contrast, decoded text can instantly be displayed on a screen, with rates already approaching the attempted speaking rate of study participants [17,22,23]. The latency of the speech-synthesis approach was lower than the previous large-vocabulary speech-synthesis BCI by a factor of 8, and communication rates were significantly improved [22]. Additionally, speech-synthesis outputs were personalized to restore the participant's pre-injury voice, a highly desired feature for this participant and others [22,54], and the participant self-reported more direct control over the streaming synthesizer compared to text-to-speech synthesis using a generic voice.

Offline, it was also shown that the decoder could operate continuously for several minutes instead of single trials lasting several seconds. This allows for the continuous operation of speech neuroprostheses using a single model. By applying the model to extended blocks of neural activity, initial steps were taken towards enabling long-form speech synthesis suitable for daily needs. The deep-learning architectures demonstrate streamability and adaptability across different articulatory silent-speech interfaces, extending beyond just ECoG in a single participant. This is important because silent speech synthesis from neural recordings without acoustic labels has only been demonstrated in a single participant using ECoG and should be generalizable to other articulatory recording interfaces.

Additionally, further improvements in decoding performance could be made from investigating closed-loop learning or entrainment, an important topic of research for motor-BCI [57,58]. Further advances in electrode interfaces enabling higher spatiotemporal resolution should also continue to improve overall system performance, including latencies [46,48]. Future approaches could include frame-wise synthesizers, which process input neural data using a sliding window and predict a single acoustic output frame per window but require a very high signal-to-noise ratio and accurately aligned overt speech reference [19,21].

Speaking seamlessly with real-time, low-latency communication at will is integral to our sense of identity and belonging, which is severely decreased in patients with anarthria. Here, a speech-decoding approach has been demonstrated that enables low-latency naturalistic spoken communication for speech and text outputs—a major step towards this goal. The approach may be refined and may ultimately be used to create a speech neuroprosthesis suitable for daily use by patients who cannot speak.

### 2.5. MATERIALS AND METHODS: OVERVIEW

### 2.5.1. Clinical-Trial Overview

This research was conducted as part of the BCI Restoration of Arm and Voice (BRAVO) clinical trial. The Food and Drug Administration approved the investigational use of the neural implant device, and the study's protocol was approved by the Institutional Review Board at the University of California, San Francisco. Informed consent was obtained from the participant, who was fully briefed on the details of the study enrollment and the potential risks associated with the study device through several discussions with the research and clinical team.

### 2.5.2. Participant

The participant, a 47-year-old at the time of her entry into the study, was diagnosed with quadriplegia and anarthria by neurologists and a speech-language pathologist after a right-pontine stroke in 2005. At the age of 30, while in good health, she experienced a sudden onset of symptoms, including dizziness, slurred speech, quadriplegia, and weakness in the muscles used for speech. Medical investigations revealed a significant pontine stroke caused by a dissection of the left vertebral artery and an occlusion of the basilar artery. During her enrollment assessment, she scored 29/30 on the Mini-Mental State Exam, missing the total score solely because her paralysis prevented her from completing a drawing task. She is capable of making a limited range of monosyllabic sounds like “ah” and “ooh” but cannot form clear words (Supplementary Note 1). In clinical evaluations, a speech-language pathologist asked her to attempt saying 58 words and ten phrases and to answer two open-ended questions within a structured dialogue. The analysis of audio and video recordings of her speech efforts indicated her intelligibility was 5% for the words and 0% for phrases and open-ended responses. She cannot use speech for communication; instead, she uses a transparent letter board and a Tobii Dynavox device to express herself (Supplementary Note 2). She provided informed consent for participation in the study and for her images to be used in demonstration videos through her transparent letter board. She spelled out “I consent” using her communication board to sign the consent documents formally and instructed her spouse to sign on her behalf.

### 2.5.3. Neural Implant

The device this research uses incorporates a high-density electrocorticography (ECoG) array (PMT) and a percutaneous pedestal connector provided by Blackrock Microsystems. The ECoG array is designed with 253 disk-shaped electrodes, organized in a grid pattern with 3 mm center-to-center spacing. Each electrode features a recording contact diameter of 1 mm and an overall diameter of 2 mm. This array was positioned subdurally on the pial surface of the brain's left hemisphere, strategically covering areas involved in speech production and comprehension, predominantly the precentral and postcentral gyri and a portion of the superior temporal gyrus. The percutaneous pedestal connector, secured to the skull in the same surgical procedure, facilitates the transmission of electrical signals from the ECoG array to an external digital headstage (CerePlex E256; Blackrock Microsystems). This digital headstage preliminarily processes and digitizes the captured cortical signals before they are sent to a computer for additional analysis.

### 2.5.4. Signal Processing

The same signal-processing pipeline was used detailed in Example 1 (above) [22,25] to extract and process common average referenced high-gamma activity (HGA) [36] and low-frequency signals (LFS) from the ECoG signals at a 200 Hz sampling rate. A 30-second sliding-window z-score was applied in real time to each ECoG channel's HGA and LFS features. All training data and online demos were collected at the participant's residence. A custom Python online data collection and task management software (rtNSR [16]) was used, which was continually maintained and used.

### 2.5.5. Task Design

Experimental paradigm: The participant silently attempted to speak phrases with short syllable-length pauses between each word precisely as instructed in Example 1 (above) [22]. The prompted text was flanked by three dots on each side, vanishing one at a time in sequence to serve as a countdown. Upon the disappearance of the last dot, the text changed to green, signaling the go-cue and prompting the participant to begin silently attempting to speak the target phrase. Following a short pause, the screen cleared, moving on to the subsequent trial.

During online speech synthesis and text decoding, the decoder began receiving neural features 500 ms before the go-cue. The speech synthesizer streamed predicted speech as she began attempting to speak the sentence silently. Meanwhile, the most recent text outputs were displayed on the screen. The effects of providing explicit instructions to participants to adjust their strategy during online decoding have resulted in conflicting results [59], so the participant was instructed to silently attempt to speak exactly as done during training regardless of the decoded outputs.

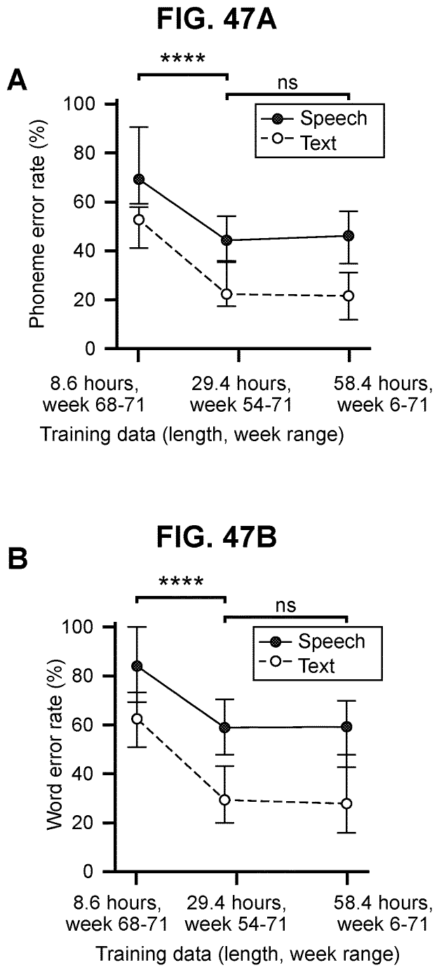

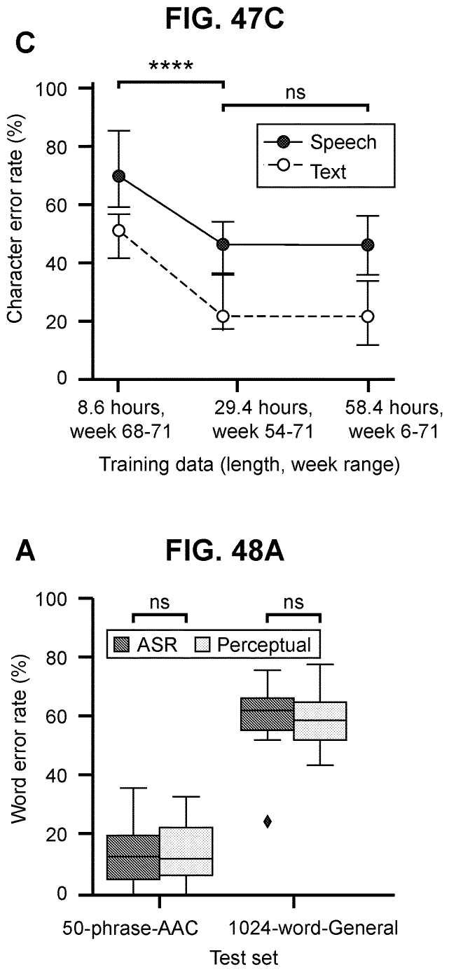

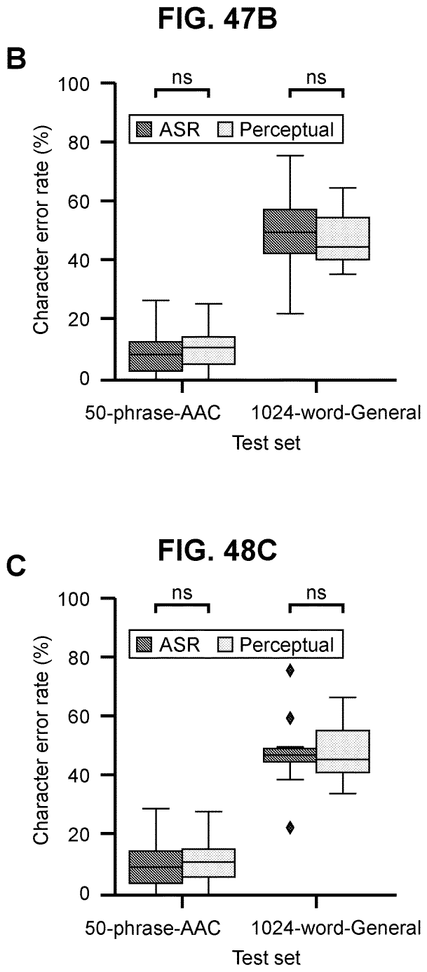

Sentence sets: A “50-phrase-AAC” sentence set and a “1024-word-General” sentence set were used in this work, with the curation being the same as in Example 1 (above) [22]. The 50-phrase-AAC contained 50 sentences of 119 unique words, with sentences chosen for clinical relevance and everyday dialogue. The 1024-word-General set contained 13,463 sentences sampled from Twitter and movie transcriptions containing 1,024 unique words. For the 50-phrase-AAC set, 11,700 trials collected over 58 weeks were used. For the 1024-word-General set, 23,378 trials (12,379 unique sentences) collected over 65 weeks of training were used. Performance was not significantly different when using data collected over the most recent 17 weeks (FIG. 47; Tables 27 and 28); however, it was decided to allow the model to learn from all available training samples. 50 trials were held out as a development set to evaluate performance and choose hyperparameters prior to online testing. 100 sentences were randomly selected from the 200 1024-word-General sentences used in the synthesis test set from Example 1 and used during the test trials. For the training and testing using the 1024-word-General sentence set, to assist the decoding models in identifying word boundaries from the neural signals without significantly compromising speed and fluency, the participant was instructed to include brief pauses of syllable length (about 300-500 ms) between words in her silent speech attempts.

FIGS. 47A-47C: Decoding accuracy by the length of training data. Median decoding accuracy is compared across different amounts of data used for training the decoder. The hours of the data are denoted along with the weeks elapsed from the date of implantation. Error bars demarcate 99% CIs. FIG. 47A: Phoneme error rates. FIG. 47B: Word error rates. FIG. 47C: Character error rates. ASR on the synthesized speech was used to measure the error rates for speech synthesis. Significant gains were observed in all metrics between 8.6 hours and 29.4 hours of training data, but no significant difference exists between 29.4 hours and 58.4 hours of training data. Performances and statistics are in Tables 27 and 28. N=10 pseudo-blocks.

TABLE 27
Decoding accuracy by the length of training data. Median
error rates (%) and confidence intervals in FIG. 47.

| Dataset | Metric | Modality | Training data hours (weak range) | | Median | 99% CI |
|---|---|---|---|---|---|---|
| 1024-word-General | PER | Speech | 58.4 | (6-71) | 46.25 | [34.78, 56.09] |
| 1024-word-General | PER | Speech | 29.4 | (54-71) | 44.57 | [35.92, 54.22] |
| 1024-word-General | PER | Speech | 8.6 | (68-71) | 69.38 | [59.12, 90.48] |
| 1024-word-General | PER | Text | 58.4 | (6-71) | 21.76 | [11.94, 31.20] |
| 1024-word-General | PER | Text | 29.4 | (54-71) | 22.45 | [17.50. 35.48] |
| 1024-word-General | PER | Text | 8.6 | (68-71) | 52.70 | [41.02, 57.85] |
| 1024-word-General | WER | Speech | 58.4 | (6-71) | 59.18 | [42.83, 70.00] |
| 1024-word-General | WER | Speech | 29.4 | (54-71) | 58.85 | [48.00, 70.60] |
| 1024-word-General | WER | Speech | 8.6 | (68-71) | 84.40 | [69.33, 100.01] |
| 1024-word-General | WER | Text | 58.4 | (6-71) | 28.00 | [16.00, 48.00] |
| 1024-word-General | WER | Text | 29.4 | (54-71) | 29.58 | [20.00, 43.33] |
| 1024-word-General | WER | Text | 8.6 | (68-71) | 62.52 | [51.00, 72.89] |
| 1024-word-General | CER | Speech | 58.4 | (6-71) | 46.02 | [36.04, 56.12] |
| 1024-word-General | CER | Speech | 29.4 | (54-71) | 46.63 | [36.24, 53.92] |
| 1024-word-General | CER | Speech | 8.6 | (68-71) | 69.89 | [59.43, 85.59] |
| 1024-word-General | CER | Text | 58.4 | (6-71) | 21.38 | [12.00, 33.78] |
| 1024-word-General | CER | Text | 29.4 | (54-71) | 22.01 | [17.51, 36.82] |
| 1024-word-General | CER | Text | 8.6 | (68-71) | 51.38 | [41.71, 56.88] |

TABLE 28
Comparisons of different training data length.

| [symbol] | [symbol] | [symbol] | [symbol] | [symbol] | [symbol] | [symbol] |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

Each comparison is a Two-sided Wilcoxon Signed-Rank test between n = 10 pseudo-blocks for 1024-word-General. 3-way Holm-Bonferroni correction for multiple comparisons was applied within metrics and modalities. [symbol]
indicates data missing or illegible when filed

### 2.5.6. Modeling

Bimodal decoder: The recurrent neural network transducer (RNN-T) is a sequence-to-sequence model designed for automatic speech recognition (ASR) tasks [41]. It enables dynamic learning of alignments between variable length input and output sequences. The RNN-T comprises three main modules: the neural encoder, the language model, and the joiner. The neural encoder, or transcriber, models the posterior of the targets from input neural signals. The language model, or predictor, is an autoregressive model which learns relationships within the target data. Finally, the joiner module combines the neural encoder and language model outputs, forming a T×L grid where T and L are the dimensions of the input neural data and target sequences, respectively. Unlike the connectionist temporal classification (CTC) loss [47], the RNN-T loss does not assume each output at a time point to be independent and is not dependent on an external language model for usable performance. During inference, multiple hypotheses are generated for plausible paths through the T×L grid, which are then searched by an RNN-T beam search algorithm (see Method 1) [41].

The bimodal decoder has two target modalities-data-driven discrete-speech units from HuBERT [42] and text tokens using Byte-Pair Encoding (BPE) and generated via SentencePiece [61]. A single shared neural encoder was trained for both modalities to utilize neural information encoded in both modalities. For each modality, a separate joiner and language model operates on the neural encoder outputs to infer probabilities of the targets from the input neural features. For discrete-speech units, 101 units extracted from the sixth layer of the HuBERT transformer encoder were used [62]. Adjacent discrete-speech units repeats were removed in a given sequence (e.g., discrete-speech unit sequence [71, 14, 14, 76, 76, 62, . . . ] would become [71, 14, 76, 62, . . . ]), resulting in an effective discrete-speech unit sampling rate of 39 Hz during training. This decouples the durations of the discrete-speech units from their contents and allows for learning a better discrete-speech unit language model. The discrete-speech unit durations are restored to 50 Hz by a discrete-speech unit duration predictor in the speech synthesis pipeline. For text, a BPE sub-word text-encoding vocabulary size of 4096 was used.

A convolutional neural network was used for the neural encoder, followed by a linear layer and a recurrent neural network. The architecture comprises two 1-D convolutional layers with 512 kernels, a kernel size 7, and a stride of 4. The recurrent portion of the neural network was composed of 3 layers of unidirectional gated recurrent units with 512 hidden units and a dropout rate of 0.5. This effectively downsamples the signal by a factor of 16 to an inference rate of 12.5 Hz, or one prediction per 80 ms. Empirically, it was found that self-attending transformer-based models did not offer substantial advantages.

For each modality, the language model comprises 4 LSTM RNN layers, each with 512 hidden units, a dropout rate of 0.3, and layer normalization. Each modality has a distinct linear layer that transforms the neural encoder outputs to decompose modality-specific features from shared neural encoder outputs. Then, the language model outputs are added to these transformed features and passed to a nonlinear function (hyperbolic tangent), followed by a linear layer with an output size corresponding to the target vocabulary. This resulted in an output distribution over 101 classes (100 discrete speech units and one blank token) for speech. Likewise, the output distribution for text is over 4097 classes (4096 sub-word tokens and one blank token). Pre-training the language model from a large speech dataset was found to be beneficial (960 hours of LibriSpeech audio; see Method 1). The language model parameters were not updated when training the models using neural activity, effectively allowing the neural decoding model to optimize only the joiner networks and neural encoder.

When streaming, ECoG features are passed to the neural encoder in 80 ms “chunks.” As model predictions were being made, RNN-T beam search was used to keep the top-K hypotheses for each iteration. While K determines the search space across L, the system can also track multiple hypotheses over T, which is helpful for text decoding. However, the model only keeps the most likely hypothesis for each time point for speech synthesis because audio chunks cannot be replayed during inference. The RNN-T beam search can still search discrete-speech unit hypotheses across L. The outputs of the RNN-T beam search are stored in internal buffers for each modality. For discrete-speech units, every 80 ms, four discrete-speech units from the speech buffer are passed as input to the speech synthesizer, and an 80 ms segment of speech is generated. The synthesized waveforms are streamed directly to a sound card via the PyAudio Python package during online decoding. For text encodings, the entire buffer is converted to text; during online decoding, it is displayed on the monitor (see Method 1).

Streaming speech synthesizer: A speech-synthesizer, or discrete-speech unit vocoder, was trained and designed to synthesize the predicted discrete-speech units into personalized, intelligible speech in increments of 80 ms. The speech synthesizer is implemented using a custom HiFi-CAR, a generative adversarial network model designed to generate high-fidelity speech waveforms from articulatory or acoustic features [63,64]. The model consisted of an input 1-D convolution, four upsampling modules, and an output 1-D convolution. Each upsampling module contains a 1-D transpose convolution for temporally upsampling the features and parallel convolutional layers with different kernel sizes. The speech synthesizer was trained on a large single-speaker dataset (LJSpeech [65]), for which voice conversion was applied to each waveform to convert the default voice into personalized audio. Specifically, a short voice clip of the participant recorded before she lost her speech ability was used to condition a voice conversion module (YourTTS [44]), which converted the LJSpeech audio waveform into personalized speech.

In addition, for the 1024-word-General set, a discrete-speech unit duration predictor was incorporated. The duration predictor module is trained to predict durations of the original discrete-speech units (i.e., before adjacent discrete-speech unit repeats were removed) to restore a consistent 50 Hz discrete-speech unit temporal resolution. This module is implemented as two 1-D convolution neural network layers with 384 hidden units and a kernel size of 3, followed by a ReLU function, layer normalization, and a dropout of 0.5. The outputs of convolutional neural network layers are then passed to a final linear projection layer to infer the duration of each discrete-speech unit. The models were trained to minimize the L1 distance between the durations of the predicted and ground-truth discrete-speech units. The discrete-speech unit duration predictor was applied to the discrete-speech units before entering the internal buffer (Method 1).

Incremental text-to-speech: As an alternative to continuous speech synthesis, an incremental text-to-speech (TTS) decoding system was developed for playing back decoded speech to the participant. A pre-trained TTS model, VITS [66], was used to synthesize the speech one word at a time as the text was predicted. During online decoding, once the text decoder predicted a new word, the complete predicted phrase was passed into the TTS system to generate an output waveform. VITS' internal word-duration predictor model was then used to identify the waveform segment corresponding to the newly predicted word. This waveform was then resampled to 16 kHz from 22050 Hz and played back to the participant via the soundcard using the same method for continuous speech synthesis. The same model checkpoint was shared between this model and the continuous speech synthesis model; however, the continuous speech synthesis language model and joiner network were disabled to speed up inference.

To match the TTS output speed with the participant's preferred speaking rate, she was asked to listen to generated waveforms of the same sentences at different audio-speed scaling factors. She was then asked which audio speed she preferred (see Method 1).

### 2.5.7. System Evaluation

Error rate calculation and perceptual evaluations: The error rate of a sequence is typically defined as the minimum number of deletions, insertions, and substitutions needed to convert a decoded transcript into the target transcript divided by the number of words in the target transcript. The units of such operations are words, characters, and phonemes for word error rate (WER), character error rate (CER), and phoneme error rate (PER), respectively. Single-trial error rates are quite variable due to differences in the lengths of the sentences, so brain-computer interface error rates are typically assessed and reported over sets of sentences rather than individual trials [24,25,67]. Sentences were sequentially parceled into “pseudo-blocks” of 10 sentences before computing metrics over their distributions, which is done by summing the edit distances between each pair of sentences and dividing by the total number of tokens (i.e., words, characters, or phonemes) in the target sentences. Phoneme sequences were determined using g2p-en [68], a grapheme-to-phoneme model.

A perceptual assessment was conducted for online speech synthesis using crowdsourced workers from Amazon Mechanical Turk. Each of the 250 online trials was independently evaluated by 12 workers (except for 26 trials where only 11 workers completed their evaluations and five trials where only ten workers completed their evaluations). Evaluators listened to the decoded speech and transcribed what they heard. The instructions and Mechanical Turk setup were identical to Example 1. To control for outlier evaluator performance, for each trial, the transcript corresponding to the median CER across evaluators was used as the final predicted transcript before computing error rate distributions (see Method 2).

Perceptual transcriptions can be costly to implement at scale, however. Since automatic speech recognition (ASR) models are now beginning to match human-level performance [69], ASR was used for offline speech synthesis evaluations. A state-of-the-art large ASR model known as Whisper [69] was used to transcribe the decoded speech for offline analyses using speech transcripts (Method 2). For online speech synthesis, word, character, and phoneme error rates were computed from transcripts generated from Whisper and found no significant difference in performance compared to perceptual transcriptions (FIG. 48; Table 29). This indicates that for the study, leading ASR models are suitable for speech-synthesis evaluation. However, more work remains to characterize its suitability in other speech-synthesis systems with different artifact profiles or speech-synthesis quality [70].

FIGS. 48A-48C: Automatic speech recognition is suitable for speech-BCI evaluation. Real-time decoding performance of speech synthesis using predicted transcripts gathered from perceptual evaluations or automatic speech recognition using the Whisper speech transcription model (Method 3). FIG. 48A: Phoneme error rates. FIG. 48B: Word error rates. FIG. 48C: Character error rates. FIGS. 48A-48C: Performances between ASR and perceptual transcripts were not significantly different, suggesting automatic speech recognition is now ready for use in speech-BCI evaluation (ns>0.05 for all metrics, Two-tailed Wilcoxon signed-rank test with 6-way Holm-Bonferonni Correction for multiple comparisons; Statistics compare n=10 total pseudo-blocks; see Table 22). Using speech recognition on the 50-phrase-AAC set, a median PER of 9.23% (99% CI [3.74, 16.4]) was observed. Median WER was 12.9% (99% CI [5.26, 25.0]), and median CER was 8.80% (99% CI [3.13, 14.3]). For the 1024-word-General set, a median PER of 49.9% (99% CI [42.1, 61.6]). Median WER was 62.4% (99% CI [53.6, 75.6]), and median CER was 48.8% (99% CI [40.2, 61.5]) was observed.

TABLE 29
Comparisons for perceptual evaluations. Each comparison is a two-sided
Wilcoxon signed-rank test between n = 15 pseudo-blocks for 50-phrase-
AAC and n = 10 pseudo-blocks for 1024-word-General. 6-way Holm-Bonferroni
correction for multiple comparisons was used for each dataset.

| Dataset | Metric | Comparison 1 | Comparison 2 | Statistic | Corrected P-value |
|---|---|---|---|---|---|
| 50-phrase-AAC | WER | ASR | Perceptual | 1.842e+04 | 1.000e+00 |
| 50-phrase-AAC | CER | ASR | Perceptual | 1.842e+04 | 1.000e+00 |
| 50-phrase-AAC | PER | ASR | Perceptual | 1.842e+04 | 1.000e+00 |
| 1024-word-General | WER | ASR | Perceptual | 1.842e+04 | 1.000e+00 |
| 1024-word-General | CER | ASR | Perceptual | 1.842e+04 | 1.000e+00 |
| 1024-word-General | PER | ASR | Perceptual | 1.842e+04 | 1.000e+00 |

Speech detection: In order to estimate when the participant was silently attempting to speak, a speech detection model was trained from neural features, with modeling parameters identical to previous demonstrations [22]. These predictions were used for decoding speed and latency calculations and were not used online.

The speech detection model was trained on the last calendar month of data from the training sets used to train the online speech-synthesis models. The 50-phrase-AAC and 1024-word-General sentence sets were used to train the online streaming speech-synthesis models. The three-class labels (silence, speech, and preparation) were automatically labeled for all data based on the task structure. Specifically, the time from the showing the target phrase until the go-cue is labeled “preparation,” while the time from the go-cue until 85% of the go-cue duration (meaning the rest of that trial where the participant silently attempted to say the phrase) is labeled “speech.” The final portion of the go-cue duration is excluded from training due to the variability in sentence lengths and ambiguity when the participant stops speaking. The inter-trial silence periods were labeled as “silence”.

The speech detection model used both LFS and HGA, causally processing these features to continuously predict the likelihood of speech at a rate of 200 Hz. The model consisted of 3 unidirectional LSTM layers containing 128, 96, and 32 hidden units, respectively. These layers are followed by a fully connected linear layer and a softmax function to determine the likelihood of preparation, speech, or silence. A modified cross-entropy loss and truncated backpropagation through time was employed to ensure the model focuses on the intended behavior rather than learning from task-specific features. This is achieved by limiting the data input to the neural data from the preceding 500 ms for each prediction.

The speech-detection model was applied to each trial in the online continuous speech-synthesis blocks for 50-phrase-AAC and 1024-word-General sentence sets. The probability distribution over the three types of speech events is then smoothed using a running window before being converted into a binary signal (identified as either speech or not) based on a predefined probability threshold. Additionally, a time thresholding technique is applied, requiring a minimum duration for changes to be recognized as an event's start (onset) or end (offset). The initial point that meets this criterion is marked as the onset or offset. A smoothing window size of 500 ms, a probability threshold of 0.5, and a time thresholding size of 500 ms was used. These parameters were chosen based on ideal parameters from Example 1.

Decoding speed and latency: To compute the onsets and offsets of the synthesized speech waveform, voice activity detection was used on the model-generated waveform [71] to get the region of time for which synthesized speech was present. Specifically, the short-term energy of the signal was computed:

$$E ( i ) = {\sum }_{n = i}^{i + c - 1} audio {( n )}^{2}$$

Here, E(i) is the energy of the i^{th} synthesized audio frame, audio(n) is the amplitude of the n^{th} sample of the synthesized speech waveform, and c is the number of samples per synthesized waveform frame. The calculation is repeated for each frame starting at sample i and advancing by a hop size of 20 ms until the end of the synthesized speech waveform. The audio was then normalized and all frames above a threshold of 0.1 were considered to be labeled as synthesized speech. The first and last detections were used for each trial as the onset and offset of synthesized speech, respectively. Text onset was defined as the emission time of the first word(s) emitted. Similarly, text offset was defined as the emission time of the last word(s) emitted.

To measure the synthesized words per minute (WPM) observed during online testing, the formula N/T was used, where N is the number of words in the synthesized waveform, and T is the time (in minutes) the participant attempted to speak. T was computed by calculating the elapsed time between the participant's detected silent speech attempt tₐₜₜₑₘₚₜ and the offset of the synthesized speech t_{synthesis offset}, yielding the resultant rate of:

$$rate = \frac{N}{{t}_{synthesis offset} - {t}_{attempt}}$$

For latency measurements, the absolute elapsed time between the participant's detected silent speech attempt and the onset of the synthesized speech was computed. The latency between the go-cue and the onset of the synthesized speech was also computed. Only speech synthesis for WPM was considered, as rapid text decoding has already been demonstrated [22]. For latency, the speech latency and text latency were considered independently. Five trials and two trials for the 50-phrase-AAC sentence set and 1024-word-General sentence set, respectively, had a detection time occurring at least 500 ms before the go-cue and were excluded from latency analyses. These trials would have occurred before the model could have received neural samples. For the 50-phrase-AAC sentence set, two trials where no audio was emitted were also excluded.

### 2.5.8. Long-Form Decoding

For the long-form decoding experiments, entire blocks of neural data was passed into the 1024-word-General sentence set model to perform continuous streaming speech-synthesis and text-decoding. Since online demonstrations used four blocks, four blocks of neural activity were independently passed into the model offline, autoregressively, and in 80 ms chunks. Each block lasted approximately 5.9 minutes. The initial input to the decoder was the data sample recorded 1 second prior to the go-cue of the block's first trial. Resetting the hidden states of the recurrent neural networks (neural encoder, language model, and joiner) was found to be beneficial if the output text encodings repeated for more than four consecutive seconds, which indicates the model has yet to generate additional outputs. Voice activity detection was used to synthesize only the active region of speech and the same 0.1 thresholds was used as for decoding latency calculations. The internal speech buffer enforces speech output in 80 ms increments, meaning the synthesized speech sounds the same as if generated in real time.

### 2.5.9. Neural Feature Evaluation

To quantify the contribution of each electrode to the online decoding, an ablation-based salience mapping method was applied [34]. The metric was calculated by determining the loss objective difference induced by occluding a specific channel in the input neural data. For each of the 253 channels, both HGA and LFS signals were set as zero for all 1024-word-General test trials. Then, the per-channel contribution value was measured by calculating the difference in loss compared to the original RNN-T loss, which was calculated with complete coverage of the channels. These differences were then averaged across trials within conditions.

The no-feedback condition consisted of 50 trials, collected on the same day as the 1024-word-General online demonstrations. The task setup for these trials was the same as during online demonstrations, except no text-decoding or speech synthesis was decoded as feedback. These sentences corresponded with the first 50 sentences from the online 1024-word-General demonstrations. In the auditory feedback condition, only the first 50 out of the 100 test trials were used to control for variations in the sentence content. For the WER evaluation, ten pseudo-blocks of 5-sentence segments were used.

### 2.5.10. Cross-Recording Modality Training and Evaluation

The test datasets from each recording modality were constrained to contain only data recorded during silent speech attempts to evaluate the model's generalizability for silent-speech synthesis. For microelectrode arrays (MEA), it began with the data split from a recent publication demonstrating high-performance open-vocabulary text decoding with a person with paralysis [23,33]. All non-silent utterances were then moved from the test set into the training or validation set. For electromyography (EMG), which uses electrodes to measure the voltage potentials on the surface of the vocal tract apparatus as a person attempts to speak, a dataset from a single healthy speaker was used [34,35]. Utterances lacking alphanumeric characters and utterances containing words that begin or end with apostrophes (which only affected the training utterances) were also filtered out. For ECoG, the speech-synthesis test set from Example 1 was used [22]. Notably, all the available training data from the released data was used. In contrast, the results reported in the previous publication only used two-thirds of the available training data due to model training-time constraints (see Method 3).

### 2.5.11. Statistics

Statistical analyses are detailed in both the figure descriptions and the above text. In summary, to compare non-paired distributions, two-sided Mann-Whitney U-tests (also known as Wilcoxon Rank-sum tests) were employed, which, importantly, do not presuppose data to be normally distributed. Two-sided Wilcoxon signed-rank tests were used for analyses involving paired data, which similarly do not assume a normal distribution of data. When relevant, the Holm-Bonferroni method was applied to adjust for multiple comparisons. P-values less than 0.05 were regarded as statistically significant. 99% confidence intervals and bootstrapped metrics were obtained by resampling the relevant data distribution 1,000 times with replacement and then calculating the desired metric for each sample.

### 2.5.7. Videos

Video 1: A demonstration of online naturalistic streaming speech synthesis and synchronized text decoding with minimal delay from brain activity. In this task, the participant silently attempts to say sentences from the 1024-word-General sentence set. Once the sentence in white turns green, she attempts to say it silently. Meanwhile, neural data is streamed to a bimodal speech synthesis and text-decoding model, which emits discrete speech units and text encodings at 80 ms increments. These intermediate features are then synthesized into personalized speech and decoded text simultaneously. The audio is streamed to the participant with low latency; meanwhile, the predicted phrase is displayed on the monitor.

Video 2: A demonstration of online streaming text-decoding and incremental text-to-speech synthesis with minimal delay from brain activity. In this task, the participant silently attempts to say sentences from the 1024-word-General sentence set. Once the sentence in white turns green, she attempts to say it silently. Meanwhile, neural data is streamed to a text-decoding model, which emits text encodings at 80 ms increments. These intermediate features are then converted into text. Once a new word is predicted, the predicted phrase, including the new word, is displayed on the monitor. In parallel, an incremental text-to-speech model synthesizes and plays the new word.

### 2.6. SUPPLEMENTARY NOTES

### 2.6.1. Supplementary Note 1

While joining the clinical trial, the participant was evaluated by a speech-language pathologist (SLP), as outlined above. The SLP found that the participant's speech impairment results from weak articulatory muscles and an inability to maintain the necessary airflow. This difficulty in maintaining airflow stems from respiratory weakness and a lack of control over airflow regulation through her vocal cords, lips, and tongue. Additionally, the participant struggles to generate the pressure to produce sounds such as plosives, fricatives, and affricates. In a vocal assessment, she demonstrated limited control over her larynx, producing effortful, monotonous sounds and pitched higher than usual. These challenges lead to speech that is uncoordinated and unintelligible.

### 2.6.2. Supplementary Note 2

The participant uses a Tobii Dynavox, a commercially available assistive-communication tool, as her primary communication method. This system includes a computer, a monitor, a camera mounted on a mobile stand, and specialized glasses. The glasses feature a reflective marker that allows the camera to track head movements, enabling the user to navigate a cursor across a digital keyboard. Selecting items is achieved by “dwelling” on them, i.e., holding the cursor steady over the selection for about a second. This setup facilitates spelling out messages. An enhanced mode offers predictive text to improve typing speed, suggesting likely follow-up text based on the initial input. For instance, typing “Hel” might prompt a “Hello” suggestion. This mode also includes a text-to-speech feature, allowing users to communicate audibly using a chosen voice. To evaluate the typing speed of this system, a task was designed where the participant would type sentences spoken by a researcher, both with and without autocomplete. Eight sentences were chosen for this purpose. The typing speed was measured in words and characters per minute, excluding the time taken for capitalization or adding final punctuation. Results showed that without autocomplete, the participant's typing speed was 8.61 words per minute and 34.1 characters per minute. With autocomplete enabled, the speed increased to 14.2 words per minute and 56.4 characters per minute. This resulted in speech synthesis latency of 23.2±3.66 seconds. The participant primarily uses this mode in her daily life.

### 2.7. METHOD 1: MODELING

### 2.7.1. RNN-T Loss

The recurrent neural network transducer (RNN-T) framework is a streamable sequence-to-sequence modeling framework designed to predict a target sequence from an input sequence where the target and the input are not temporally aligned. The RNN-T is trained by maximizing the likelihood of target (Y⊆R^{U×d}) given input (X⊆R^{T×D}) while jointly finding the alignment (d and D are the sizes of input data and target data, respectively; At the same time the RNN-T can operate on arbitrary lengths of both input and target sequences, T and U were set to denote the lengths of input and target for simplicity.). The RNN-T consists of three main modules: a transcriber, a predictor, and a joiner. The transcriber and the predictor extract features from the input sequence x=(x₁, x₂, . . . , x_{T})∈X, and the one-time-point shifted target sequence y=(Ø, y₁, y₂, . . . , y_{U-1})∈Y, respectively, where Ø is a blank token. Note that the predictor model is supposed to have a causal architecture that only allows information to flow forward in time (e.g., unidirectional RNN), while there is no architectural limitation on the transcriber.

Let fᵢ∈f₁, f₂, . . . , f_{T} be the t-th time-point of the transcriber output, and gᵤ∈g₁, g₂, . . . , g_{U} be the u-th time-point of the predictor output. Given the causal architecture of the predictor, gᵤ represents y_{1:u-1}, and ft represents x that can be further prescribed to be x_{1:t} by forcing the transcriber to be causal. The causal transcriber is a requirement for the streaming capability of RNN-T, which enables inference without the need for future context. The joiner then combines fr and gᵤ to represent P(Y=y|fₜ, gᵤ), which is denoted as h_{t,u}[y]. The inference (or emission) is made by choosing y that maximizes h_{t,u}[y] at each input time point, t, where a beam search is commonly utilized to reduce the search space (see RNN-T beam search section).

As the input and target sequence are not aligned, multiple emissions (or alignments) patterns should be considered. For example, when predicting (y₁, y₂) from (x₁, x₂, x₃), both (y₁, Ø, y₂) and (Ø, y₁y₂) mean the same output. The output of the joiner forms a T×U grid, on which the probability of alignment between x and y can be efficiently calculated. Let a be a possible alignment between x and y, and A be the set of all possible alignments, α∈A. The probability of y given x is:

$$P ( y \vert x ) = {\sum }_{α ∈ A} P ( α \vert x , y ) P ( y \vert x , α )$$

For simplicity, let α:=P(a|x, y), and α_{t,u} represents alignments by the t and u time-points on the grid. To efficiently compute the summation, α_{t,u} was computed for every possible pair of t and u using:

α_{t,u}=α_{t-1,u}h_{t . . . 1,u}[Ø]+α_{t,u-1}h_{t,u-1}[yᵤ₋₁]

Then, the P(y|x) can be simplified as α_{Y,U}h_{T,u} [Ø]. The loss function for training RNN-T is defined as the negative log of P(y|x), which is averaged in a batch sampled from the training dataset. PyTorch's RNNTLoss function was utilized to calculate the RNN-T loss efficiently.

### 2.7.2. RNN-T Beam Search

The beam search algorithm was used to predict output units from the RNN-T's joiner emissions. The algorithm was adopted from [41] as Algorithm 1. The beam search algorithm searches for the optimal sequence of tokens by extending each of the hypotheses in the beam, B, while keeping the search space at each time point limited to the top K hypotheses. Since multiple emissions are allowed per time step, hypotheses per time step is flexible but must be less than or equal to M. At the end of the iterations within each time point; the beam is further truncated to the top J hypotheses that are carried over to the next time point. K is set as 5 for speech and 20 for text. J is set as 1 for speech (given that speech that has already been synthesized cannot correct for) and 10 for text. M is set as 20 for both modalities. The argsort operation in Algorithm 1 denotes sorting the elements for the given term in descending order.

Algorithm 1 Beam Search Decoding

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

### 2.7.3. Bimodal Pretraining

The predictors and joiners of the brain decoder were pre-trained with a large speech dataset, LibriSpeech, that has 960 hours of speech derived from English audiobooks [72]. As these modules cannot be trained independently, the whole RNN-T was trained with RNN-T loss on automatic speech recognition (ASR) tasks. The input was speech audio for both speech and text; the target was either subword BPE text tokens or discrete-speech units extracted from HuBERT. For speech, a transducer model was trained to predict the discrete-speech units from speech and used the RNN-T loss to optimize the parameters. The architecture of the transcriber consisted of two convolutional layers with 512 hidden units, kernel size of 3, and stride of 2, followed by a unidirectional LSTM with three layers of 512 hidden units. The joiner and predictor have the same architecture as described above. Before being fed to the transducer, the Mel spectrogram with 80 dimensions is extracted from a 16 kHz waveform with a hop length of 160, resulting in a spectrogram sampled at 100 Hz. The transducer is trained with Adam optimizer [73] with a learning rate of 1e-3, β1=0.9, and β2=0.999, updated for 200K steps using a batch size of 20. For text, the checkpoint was retrieved provided by PyTorch, which implemented the RNN-T approach for ASR proposed by [39]. For both modalities, the predictors were employed in the streaming decoding and frozen while training the neural transducer. Also, the trained joiner weights were used to initialize the joiner weights in the ECoG transducer.

### 2.7.4. Neural-Decoder Training Details

For the 1024-word-General model, initial training used data from sessions collected up to two days before the real-time experiments. Subsequently, all sessions were included, including those from one day before the final experiments. The training utilized the Adam optimizer, with settings at a learning rate of 1e-4, β1=0.9, β2=0.999, and a batch size of 64. Initially, the model underwent 745 epochs of training, and after adding additional data, it was updated for an additional 265 epochs. The training was performed on three NVIDIA RTX A6000 GPUs, and real-time inference was performed on a single NVIDIA Tesla V100 GPU.

Data augmentation was applied to enhance model robustness and generalization.

Following the methodology described by Example 1, a random channel drop was implemented, applying it with a 0.75 probability to each training sample. Each channel (HGA and LFS) might be dropped within this framework, with chances uniformly drawn from the range [0.3, 0.6]. Additionally, a temporal window was selected from each training sample, with its size uniformly chosen between 70% and 90% of the sample's original length.

For the 50-phrase-AAC model, the CNN has one convolutional layer with 512 hidden units, a kernel size of 4, and a stride of 4. A unidirectional LSTM replaces the GRU for the RNN component and is comprised of 3 layers with 512 hidden units, each with a dropout rate of 0.4. The modality-specific linear layers used before combining language model outputs were not included. The channel drop probability was set at 0.5, with a fixed drop rate of 0.2 per channel. The temporal jitter window for this model was set between 90% and 100% of the sample length. A batch size of 32 was used and the model was trained for 100 epochs on a single NVIDIA RTX A6000 GPU. The speech synthesizer did not employ duration prediction.

For all brain-decoding models, training audio was synthesized from the text prompts using VITS [66]. A single speaker model trained on LJSpeech dataset was used [74]. The synthesized audio was then used to extract discrete speech units using HuBERT [75].

### 2.7.5. Synthesizer Training

A speech synthesizer was trained and designed to generate personalized, intelligible speech from the predicted discrete-speech units. The speech synthesizer consists of a duration predictor and a discrete-speech-unit vocoder. The duration predictor was trained to predict the duration of de-duplicated units to restore the original speech-unit sampling rate (50 Hz). The discrete-speech-unit vocoder was implemented as HiFi-CAR, a generative adversarial network model that generates a high-fidelity speech waveform from articulatory or acoustic speech features [63]. For training the speech synthesizer, a sizeable single-speaker speech corpus was used (LJSpeech [74]; 20.3 hours for training). Every clip was converted in LJSpeech to the participant's voice to make a personalized vocoder that generates speech in the participant's pre-injury voice. A short 3-second far-field voice clip provided by the participant was used, which was recorded before her loss of speech ability. The participant's voice clip was first denoised and enhanced by Adobe Enhancer and then prompted YourTTS to convert every clip in the LJSpeech corpus into personalized audio [44].

The HiFi-CAR is an autoregressive extension of HiFi-GAN [63,64,76]. The HiFi-GAN generator was implemented as 1-dimensional (1D) convolutional neural networks (CNNs) that consisted of an input 1D convolution followed by four upsampling modules and an output layer. Each upsampling module contains a Leaky ReLU (0.1 negative slope), a ConvTranspose1D for upsampling, and a Multi-Receptive Field Fusion (MRF) [64]. The MRF was implemented with residual convolutional layers, each consisting of a Leaky ReLU and a Conv1D with various sizes of the receptive field that was configured by kernel size of [3, 7, 11] and dilation of [1, 3, 5] resulting nine combinations in total. The upsampling modules had [8, 5, 4, 2] upsampling factors times respectively. The number of hidden unit channels was halved by each upsampling module, implemented in the ConvTransposed layer within the module, which had kernel size of [16, 10, 8, 4], respectively, for four modules. For the input convolution, kernel size 7, output channel of 255, with a padding of 255 was used. For the output convolution, kernel size 7, out channel of 1, with a padding of 255 was used. The final output was fed into another Leaky ReLU (0.1 negative slope) and an output convolution. Before feeding into the input layer, the discrete-speech units were converted to 768-dim learnable embedding using an embedding lookup dictionary.

The HiFi-CAR extension added conditioning inputs from previously generated wave samples, which was demonstrated to improve the quality of synthesis [63,76]. This assumes a streaming generation scenario, where the waveform is generated chunk-by-chunk progressively. The previous 512 predicted audio samples were fed into an audio encoder (32 ms, given the 16K audio sampling rate). The audio encoder consisted of four fully connected layers with 512 hidden units and leaky ReLU (0.1 negative slope) activation, followed by an output linear layer with 512 output channels. The output of the audio encoder was concatenated to the discrete-speech-unit embedding before passing this to the input layer of the HiFi-GAN.

The vocoder was trained with the generative adversarial neural networks (GAN) paradigm, a generative modeling framework where the generator is trained to fool a jointly trained discriminator that detects real/fake data [77]. To achieve high-fidelity waveform synthesis, the HiFi-GAN utilized two types of discriminators: a multi-period discriminator (MPD) and a multi-scale discriminator (MSD) [63,64]. The MPD was designed to discriminate between real/fake waveforms chunked with different periods, which consisted of CNN discriminators. For each discriminator, the input waveform with length T was reshaped to (P, T/P), where P is the period specified in each model in the MPD. The discriminator was implemented with 2D CNN with five residual blocks with downsampling factors of [3, 3, 3, 3, 1], respectively, where the kernel size was set as (5, 3) for all layers except the final output convolution that had a kernel size of (2,1). The final output was summed to represent an indicator of the genuinity of the input sample. Five different periods of [2, 3, 5, 7, 11] were used, and the discriminator for each period operated independently. On the other hand, the MSD was designed to operate on the multiple scales of the input waveform, which was packaged with multiple CNN discriminators with different scale configurations. Each CNN discriminator with a specified scale, S, processed a waveform downsampled by the factor of S by averaging every S time-point chunk. Each discriminator in the MSD was implemented with 1D CNN with an input layer with output channels of 128 and kernel size of 15; 5 convolutional layers with channels of 128, kernel size of 41, groups of 4, and a stride of [2, 2, 4, 4, 1] respectively; and an output layer with two convolutional layers with output channels of 256, kernel size of [5,3] for each layer, and stride of 1. Then, the final output was summed up as an indicator of real/fake, three different scales of [1, 2, 4] were used, and the discriminator for each scale operated independently. The weights in the MPD and MSD were normalized to have a unit norm.

The adversarial loss was defined as a min-max game between the generator and the discriminator [77]. The discriminator, D, was trained to discern generated audio from the ground truth y, and the generator, G, was trained to fool the discriminator (x is the corresponding input feature of y, sg means stop-gradient and thus no weight update).

L_{A}=−E[−D(G_{sg}(x)+D(y)]+E|D_{sg}(G(x)]

Here, Mean Squared Error was used between the discriminator outputs and 1 for D, following [64].

For accurate synthesis, a reconstruction loss, L_{M}, was calculated between ground truth and generated waveform on an acoustic space represented by Mel spectrogram, minimizing the L1 distance. To enhance fidelity, a feature matching loss, L_{F}, was also adopted, which was designed to minimize the L1 distance of penultimate features in the discriminators between the ground truth and the synthesized waveform. This loss was calculated and summed up in all layers of the MPD and MSD. The final loss, L_{G}, is a weighted sum of discriminator loss and feature matching loss.

L_{G}=L_{M}+45*(2*L_{F}+L_{A})

Following [78]; the duration predictor was implemented as two 1D CNN layers with 384 hidden units, kernel size of 3, followed by ReLU function, layer normalization, and dropout of 0.5. The outputs of CNNs are then passed to a final linear projection layer to infer the duration of each unit in the inputs. For the duration output of the generator, which is in log space, the ground truth duration was first converted into log space and then the MSE loss between the two was computed.

During inference, the duration predictor predicted the duration of each input discrete speech unit. Then, each unit was de-duplicated by the predicted duration to restore the 50 Hz sampling rate. The output of the duration was then multiplied by a constant factor of 7 to simulate the participant's slow rate of attempted speech and minimize WER. Finally, the HiFi-CAR generator synthesized audio from the expanded units. The duration predictor was independently trained from the HiFi-CAR.

Adam was used as the optimizer for the generator with β=(0.5, 0.9) and no weight decay. MultiStepLR was used as the learning rate scheduler, decreasing by a factor of 2 for every milestone of 80 k, 160 k, 320 k, and 400 k iterations. A batch size of 32 was used for each iteration, and a sample in the batch had 260 ms audio (8.32 s per batch). The best model is selected based on the lowest L_{M} evaluated on held-out validation data (1.23 hours of data). When tested on held-out 2.41 hours of test data, the synthesized speech was highly intelligible, and Whisper ASR (“openai/whisper large v2”) was applied to the audio result.

### 2.7.6. Incremental Text-to-Speech

For the design of the incremental text-to-speech (TTS) pipeline, the VITS TTS model trained on the single-speaker LJSpeech dataset was used [74]. Ideally, one would synthesize a single word at a time. However, most TTS models are trained on sentence-level corpora, and thus, single-word generations can often sound degraded or unintelligible. To work around this, the current, complete predicted phrase was passed into VITS for each word-level text generation during streaming text decoding. Since VITS explicitly models the expected duration of each word in the output sentence, their duration predictor was use to identify and segment only the new part of the phrase if at least one new word has been generated. For example, within a trial, if the previous decoded phrase was “How are,”. The new update corresponds to “How are you,” or “How are you today,”, these complete phrases were synthesized and then “you,” or “you today,” was segmented and played back respectively.

To match the output TTS with the preferred speaking rate of the participant, the VITS length scaling factor was adjusted to 1.61. This factor scales the modeled duration predictors by a constant value and hence can be used to adjust the speed of the waveform naturally. The participant was asked to listen to 10 VITS TTS-generated waveforms with even-spaced length scaling factors from 0.50 to 3.00 to find this parameter. She was asked to select which waveform most represents her preferred speaking rate during silently attempted speech (miming) with pauses. The scaling factor chosen (1.61) was then used during inference.

### 2.8. METHOD 2: EVALUATION

### 2.8.1. Perceptual Assessment

Perceptual evaluations were conducted through crowdsourcing on Amazon Mechanical Turk, with each experiment from the real-time 1024-word General and 50-phrase AAC test sets reviewed by 12 participants, except for 26 trials reviewed by only 11 workers and five trials reviewed by only ten workers. Each review involved listening to the decoded audio waveform, after which participants were asked to transcribe what they heard. To prevent the workers' time from being spent listening to pre-speech silence, the onsets and offsets of the waveform were identified. The waveform was then segmented to include only the active portions before uploading it for perceptual evaluations. The detailed instructions were as follows:
Please listen to the audio and write down what you hear. Many of the clips may be difficult to hear. If this is the case, write whatever words you are able to make out, even if it does not form a complete expression. If you are not sure about a word, please only include your guess in your transcription if you feel that you are over 50% confident that your guess is correct. Otherwise, exclude the guess from your transcription. If you cannot make out any words, leave the entry blank. You may listen as many times as needed.

The transcript corresponding to the median worker character error rate was used as the predicted transcript for that trial. Before computing metrics, punctuation was removed, the transcripts were lowercased, and the terms “N/A” or “Inaudible” were removed.

### 2.8.2. Perceptual Assessment

A state-of-the-art large automatic speech recognition (ASR) model known as Whisper [69] was used to transcribe speech-synthesis results in the manuscript. The “openai/whisper-large-v2” checkpoint was downloaded from Hugging Face and the decoded audio waveforms were passed at 16 kHz into the model to obtain a transcript. It was observed that for a tiny percentage of the transcripts, typically from the chance distributions, Whisper outputs an excessively long transcript either in terms of the number of words or the number of characters, with a (truncated) example being:
theyressssssssssssssssssssssssssssssssssssssssssssssssssssssssss

For this reason, a rule-based modification of the output transcripts was implemented. Specifically, all transcripts were truncated to be a maximum of 9 words and each word was truncated to have a maximum of two repeated characters. In this example, the output transcript would be truncated to “theyress”. This allows for a better assessment of the transcript quality as decoded from neural data as opposed to artifactual generations from the ASR model. For analyses aimed at generalization across modalities, this 9-word constraint was removed because the target sentences were much longer than nine words for these analyses.

### 2.9. METHOD 3: MULTIMODALITY

### 2.9.1. Dataset Processing

The electrocorticography (ECoG) data for the multimodal generalization analysis was obtained from a dataset from the participant [22]. The neural signal processing in [22] was identical to the neural signal processing used in this Example. The speech-synthesis test set, which tested was used on 200 unseen 1024-word-General trials. [22] used a model which, at the time, required several days to converge, so only used 6,449 1024-word-General trials during training of the speech-synthesizer models were used (approximately ⅔ of the total data collected in the study). For this Example, all available unseen 1024-word-General sentences provided (excluding sentences from the same session as the test session) were used (9,368 training sentences). 100 sentences were randomly selected from these training sentences and held for use as a validation set.

The microelectrode array (MEA) data was obtained from the competition dataset introduced in [23] and released at [33]. It contains data collected in both (attempted) vocalized and silent speech modes from a person with paralysis. This dataset consists of a total of 9680 samples and has a provided split of 8800 training samples and 880 test samples. The utterances in this dataset were from the open-vocabulary Switchboard and OpenWebText corpora and a fixed 50-phrase set that was only used for training. To evaluate generalization on the silent speech task, only the silent speech subset of the provided test set was used for evaluation, and all the other samples were moved to the training dataset. Randomly selected 100 silent speech samples were also removed from the training set to form a validation set. This resulted in 9420 training samples (7560 vocalized and 1860 silent), 100 validation samples (all silent), and 160 test samples (all silent). The training features used for this task were the spike band power and threshold crossing counts, with a threshold of −3.5×RMS, from 128 electrodes placed in the ventral premotor cortex (area 6v) as computed and filtered in [23]. These features were then z-scored within blocks to account for drifts in feature means across blocks.

The electromyography (EMG) data was obtained from [79] that proposed an EMG-based “voicing silent speech” paradigm. The EMG captured surface articulatory information collected from a participant in two different tasks: voiced and silent [34]. This dataset contains 8653 open-vocabulary samples from public domain books. All of the silent samples were parallel voiced samples collected on the same utterances; there were also additional voiced samples that did not have parallel silent recordings. Unlike [79,35,80], it was not attempted to time-warp the voiced and silent sequences or use the voiced audio as the ground truth when training the model. Instead, ground truth audio was generated from the text transcripts provided in the dataset; this procedure was consistent with the ECoG and MEA modalities and ensured that the model did not rely on any aligned ground truth speech. Additional filtering and processing were required to ensure the quality of this generated audio. 19 samples were removed because the utterances had no alphanumeric characters (e.g., or “* * * * *” or “.”) and an additional 246 samples were removed which contained ambiguous words that began or ended with apostrophes (e.g., ‘eat or ha’). The utterance texts were cleaned up for the remaining samples without changing their content. For instance, numbers and Roman 14 numerals were converted into word form, and currency amounts were reformatted (e.g., $12 would become “twelve dollars”). To split the data into training, validation, and test sets, the data splits provided in [79] were used to begin with, which had 200 validation samples and 100 test samples. After applying the utterance filtering described above and removing duplicated samples, the validation set had 196 validation samples and 98 test samples, all containing silent speech. The parallel voiced samples were removed from the remaining data corresponding to the samples in the validation and test sets, leaving 7800 training samples (6529 voiced, 1271 silent). The features, which consisted of eight electrodes sampled at 1000 Hz, had the following signal processing steps applied: band-stop filters at harmonics of 60 Hz, a third-order high-pass Butterworth filter at 2 Hz, a subsampling from 1000 Hz to 689.06 Hz, a rescaling to unit values of 20 μV, and a soft de-spiking with a scaled hyperbolic tangent function by [80]. All training and validation utterances were removed with input EMG lengths over 20000 during training due to memory constraints. This length limit was did not apply to filter the test set.

### 2.9.2. RNN-T Training Procedure

For the ECoG transcriber, all training and model hyper-parameters were the same as those used for real-time streaming. For other modalities, modality-specific transcribers were designed and the joiners and predictors were used in the same way as for real-time streaming.

For the streaming MEA transcriber, the same Gaussian smoothing unfold operation was used as in [23]. A size six kernel and a stride of 2 were chosen to unfold the input neural features that increased the input dimensionality from 256 to 1536. Note that the session layer proposed in [23] was not included for streaming as it was found the session layer harmed streaming performance. After the unfolding, a linear projection was used followed by a dropout of 0.2 to project from 1536 to 512 dimensions, which was the input dimensionality for an RNN. For this, a 3-layer unidirectional GRU was chosen followed by a 1D CNN with 512 input channels, 512 output channels, kernel size of 3, and stride of 2. This was followed with a linear layer followed by a dropout of 0.2 and a second linear layer with 512 units to output the final encoding outputs with 1024 units. 0.7 and 0.3 were chosen as the weights of the discrete-speech-unit RNN-T loss and text-encoding RNN-T loss, respectively.

For the full-context MEA transcriber, the same session layer as in [23] was added back and the GRU was changed to be bidirectional. The 1D CNN after the BiGRU was also adjusted to have 1024 input channels, 1024 output channels, a kernel size of 3, and a stride of 2. For all linear layers after the 1D CNN, an input and output unit dimensionality of 1024 were used. Everything else was kept the same as the streaming MEA transcriber.

For the streaming EMG transcriber, four ResBlocks were used following the implementation in [80], with a hidden dimension 1024 for all of them and kernel size parameter of [1, 2, 2, 2]. This is followed by a linear projection and a dropout of 0.2 to project from 1024 to 768 dimensions. The output is fed into a 4-layer unidirectional GRU with 768 hidden dimensions. The GRU is then followed by a 1D CNN with 768 input channels, 768 output channels, a kernel size of 12, and a stride of 8. This was followed with a linear layer, a dropout of 0.2, and 15 a second linear layer with 768 units. The second linear layer has an output unit of 1024. 0.7 and 0.3 were chosen as the weights of the discrete-speech-unit RNN-T loss and text-encoding RNN-T loss, respectively.

It was found that unidirectional GRUs perform better than BiGRUs offline for the full-context EMG transcriber. 3 Resblocks were used, each consisting of a 1D CNN and batchnorm1D with residual connection. ReLU was used as the activation function after the residual connection. For the three 1D CNN modules, kernel sizes of [7, 7, 2], strides of [4, 4, 2], and output channels of [512, 512, 512] were chosen, respectively. The first 1D CNN has an input channel equal to the number of channels in EMG, which is 8. The 1D CNN modules were followed by a linear projection and a dropout of 0.2 to project from 512 to 512 dimensions. The output is fed into a 3-layer unidirectional GRU with a hidden dimension of 512. This was followed with a linear layer followed by a dropout of 0.2 and a second linear layer with 512 units. The second linear layer has an output units of 1024. An equal weight of 1.0 and 1.0 were chosen for the discrete-speech-unit RNN-T loss and text-encoding RNN-T loss, respectively.

For all three models, identical beam search parameters were used as for the real-time 1024-word-General beam search. For the RNN-T full-context experiments, the streaming buffer was disabled, and for ECoG and MEA, the neural encoder was changed to be bidirectional.

### 2.9.3. CTC Training Procedure

Given an input sequence Xₙ=(x₁, x₂, . . . , x_{T})∈X and an output sequence Yₙ=(y₁, y₂, . . . , y_{U}) E Y, the Connectionist Temporal Classification (CTC) loss assumes a monotonic alignment between the two sequences. It requires U≤T where U is the length of the output sequence and T is the length of the input sequences. Denote & as one possible alignment between Xₙ and Yₙ at time step t, and let A be the set of all possible alignments between Xₙ and Yₙ. The standard CTC loss, as defined in [47], is the negative log-likelihood of the sum of the probabilities of all possible alignments between Xₙ and Yₙ, marginalized over the set of valid alignments:

$$\begin{matrix}{L}_{CTC} = - log P ( {Y}_{n} \vert {X}_{n} ) \\ = - log {\sum }_{α ∈ A} {\prod }_{t = 1}^{T} P ( {α}_{t} \vert {X}_{n} )\end{matrix}$$

The CTC loss assumes conditional independence, where each output yᵤ is conditionally independent of the other outputs given the input sequence Xₙ. For text decoding, this assumption necessitates a language model to provide contexts for decoding sensible texts, such as “he ate an apple” instead of “cc cight an app.”

Using the CTC loss, a CNN-Transformer with two output heads was trained for silent speech synthesis and text-decoding from electrocorticography (ECoG), microelectrode arrays (MEAs), and electromyography (EMG) input features. For all three datatypes—ECoG, MEA, and EMG-the core processing architecture involves initial signal modification followed by the application of a transformer model. Adam was used as the optimizer for all models 16 with beta (0.5, 0.9), the learning rate as 0.0001, and 0.0 weight decay. MultiStepLR was used as the scheduler with gamma as 0.5. A CTC beam search was used to find the most likely hypotheses from the CTC emissions matrix, but no external language model was applied.

For ECoG, the input was first passed through a 1D CNN with an input channel of 506, an output channel of 1024, a kernel size 6, and a stride of 6. This convolution is followed by a linear layer that projects the channel dimension from 1024 to 1024 units. After the linear layer, a 6-layer, 8-head Transformer was used with a 1024 hidden dimension, a 3072 feedforward dimension, 0.2 dropout rate, and a relative-positional embedding that utilizes a relative positional distance of 100. The transformer's output was divided into two heads, one for speech and the other for text. The speech head consisted of a ConvTranspose1D with an input channel of 1024, an output channel of 1024, a kernel size 6, and a stride of 6. This was followed by a linear projection layer that reduced the channel from 1024 dimensions to 101 dimensions and a final LogSoftmax layer for converting the output at each time step into a log probability distribution. The text head consisted of a linear projection layer that reduced the channel from 1024 dimensions to 41, followed by a LogSoftmax layer. The CTC loss function implemented by PyTorch was used, setting the “zero infinity” parameter to “true.” The blank token index for the speech output was 100, while the text output was 0. The final loss was a weighted sum of the CTC losses for speech and text outputs, with weights of [0.8, 0.2], respectively. For this model, the milestones were set for the multi-step learning rate scheduler to [8000, 15000], where the learning rate was halved every milestone, and trained for a maximum of 20000 steps, where each step represents one forward pass and back-propagation of one batch of data with a batch size of 16. The validation set was evaluated every 100 steps and the final model was selected based on the lowest validation loss.

For the MEA processing, the input was initially subjected to the same Gaussian

smoothing, session layer, and unfold operation as in [23]. This output was passed through another linear projection layer, reducing the channel dimension from 1536 to 1024. Following this, a 6-layer 501 8-head Transformer architecture was utilized, featuring a 1024 hidden dimension, a 3072 feedforward dimension, a dropout rate of 0.2, and relative-positional embedding utilizing a distance 100. The transformer's output then diverged into two distinct pathways: one for speech and another for text. The speech pathway utilized a ConvTranspose1D operation (input and output channels of 1024, kernel size of 6, and stride of 2), followed by a linear projection layer that reduces the dimensionality from 1024 to 101 and concluded with a LogSoftmax layer for generating categorical log probability distributions per time step. The text pathway employed a 1D CNN (input and output channels of 1024, kernel size of 2, stride of 2), followed by a dimension-reducing linear projection layer and a LogSoftmax layer. The CTC loss function from PyTorch was used for loss calculation, with the “zero infinity” parameter enabled to handle padding. The blank identifiers were set to 100 for speech and 0 for text outputs. The final model loss was a weighted combination of the CTC losses for speech and text, with weights of 0.6 and 0.4, respectively. The learning rate was halved after 5000 steps, and the model was trained with 10000 steps with a batch size of 16. While training, the model was evaluated every 100 steps on the validation set. After the training, the model checkpoint with the lowest validation loss was selected.

For the EMG, the model was implemented with CNN feature extractor and Transformer encoder [80]. The CNN had 4 Residual Blocks with 768 hidden dims, kernel size of 3, and downsampling strides with [1, 2, 2, 2], respectively. The Transformer encoder had six layers of an 8-head self-attention network followed by a feedforward layer with 3072 hidden units and a 0.2 dropout rate. The input features were concatenated with relative-positional embeddings. The transformer outputs were then fed into two separate branches: one for speech and one for text. Each branch consisted of a ConvTranspose1D to upsample the input by the factor of 2, followed by a linear classifier. The log softmax was applied to infer categorical log probability distributions at each time step for each modality.

The loss computation uses the CTC loss function from PyTorch's functionals with “zero infinity” set to true. The blank identifiers for speech and text outputs are 100 and 0, respectively. The final model loss is the weighted sum of the CTC losses for speech and text, with weights [0.9, 0.1]. The learning rate was halved every after 6000, 10000, and 15000 steps, and the model was trained with 20000 steps with a batch size of 4. While training, the model was evaluated every 500 steps on the validation set. After the training, the model checkpoint with the lowest validation loss was selected. During training and validation, utterances with EMG lengths above 20000 are excluded; however, all utterances are retained during testing, regardless of EMG length.

### 2.10. REFERENCES

The numbering related to the following references apply with respect to the experimental results presented in Example 2:
[1]_Felgoise, S. H., Zaccheo, V., Duff, J. & Simmons, Z. Verbal communication impacts quality of life in patients with amyotrophic lateral sclerosis. Amyotroph. Lateral Scler. Front. Degener. 17, 179-183 (2016).
[2]_Huggins, J. E., Wren, P. A. & Gruis, K. L. What would brain-computer interface users want? Opinions and priorities of potential users with amyotrophic lateral sclerosis. Amyotroph. Lateral Scler. 12, 318-324 (2011).
[3]_Branco, M. P. et al. Brain-Computer Interfaces for Communication: Preferences of Individuals With Locked-in Syndrome. Neurorehabil. Neural Repair 35, 267-279 (2021).
[4]_Peters, B., O'Brien, K. & Fried-Oken, M. A recent survey of augmentative and alternative communication use and service delivery experiences of people with amyotrophic lateral sclerosis in the United States. Disabil. Rehabil. Assist. Technol. 1-14 (2022) doi:10.1080/17483107.2022.2149866.
[5]_Peters, B. et al. Brain-Computer Interface Users Speak Up: The Virtual Users' Forum at the 2013 International Brain-Computer Interface Meeting. Arch. Phys. Med. Rehabil. 96, S33-S37 (2015).
[6]_Chang, E. F. & Anumanchipalli, G. K. Toward a Speech Neuroprosthesis. JAMA 323, 413 (2019).
[7]_Bouchard, K. E., Mesgarani, N., Johnson, K. & Chang, E. F. Functional organization of human sensorimotor cortex for speech articulation. Nature 495, 327-332 (2013).
[8]_Carey, D., Krishnan, S., Callaghan, M. F., Sereno, M. I. & Dick, F. Functional and Quantitative MRI Mapping of Somatomotor Representations of Human Supralaryngeal Vocal Tract. Cereb. Cortex 27, 265-278 (2017).
[9]_Lotte, F. et al. Electrocorticographic representations of segmental features in continuous speech. Front. Hum. Neurosci. 09, 1-13 (2015).
[10]_Chartier, J., Anumanchipalli, G. K., Johnson, K. & Chang, E. F. Encoding of Articulatory Kinematic Trajectories in Human Speech Sensorimotor Cortex. Neuron 98, 1042-1054.e4 (2018).
[11]_Dichter, B. K., Breshears, J. D., Leonard, M. K. & Chang, E. F. The control of vocal pitch in human laryngeal motor cortex. Cell 174, 1-11 (2018).
[12]_Herff, C. et al. Brain-to-text: decoding spoken phrases from phone representations in the brain. Front. Neurosci. 9, 1-11 (2015).
[13]_Mugler, E. M. et al. Direct classification of all American English phonemes using signals from functional speech motor cortex. J. Neural Eng. 11, 035015-035015 (2014).
[14]_Makin, J. G., Moses, D. A. & Chang, E. F. Machine translation of cortical activity to text with an encoder-decoder framework. Nat. Neurosci. 23, 575-582 (2020).
[15]_Sun, P., Anumanchipalli, G. K. & Chang, E. F. Brain2Char: a deep architecture for decoding text from brain recordings. J. Neural Eng. 17, 066015 (2020).
[16]_Moses, D. A., Leonard, M. K. & Chang, E. F. Real-time classification of auditory sentences using evoked cortical activity in humans. J. Neural Eng. 15, (2018).
[17]_Card, N. S. et al. An Accurate and Rapidly Calibrating Speech Neuroprosthesis. http://medrxiv.org/lookup/doi/10.1101/2023.12.26.23300110 (2023) doi:10.1101/2023.12.26.23300110.
[18]_Herff, C. et al. Generating Natural, Intelligible Speech From Brain Activity in Motor, Premotor, and Inferior Frontal Cortices. Front. Neurosci. 13, (2019).
[19]_Anumanchipalli, G. K., Chartier, J. & Chang, E. F. Speech synthesis from neural decoding of spoken sentences. Nature 568, 493-498 (2019).
[20]_Angrick, M. et al. Speech synthesis from ECoG using densely connected 3D convolutional neural networks. J. Neural Eng. 16, 036019 (2019).
[21]_Wairagkar, M., Hochberg, L. R., Brandman, D. M. & Stavisky, S. D. Synthesizing Speech by Decoding Intracortical Neural Activity from Dorsal Motor Cortex. in 2023 11th International IEEE/EMBS Conference on Neural Engineering (NER) 1-4 (IEEE, Baltimore, MD, USA, 2023). doi:10.1109/NER52421.2023.10123880.
[22]_Example 1: A High-Performance Neuroprosthesis for Speech Decoding and Avatar Control (Above).
[23]_Willett, F. R. et al. A high-performance speech neuroprosthesis. Nature 620, 1031-1036 (2023).
[24]_Moses, D. A. et al. Neuroprosthesis for Decoding Speech in a Paralyzed Person with Anarthria. N. Engl. J. Med. 385, 217-227 (2021).
[25]_Metzger, S. L. et al. Generalizable spelling using a speech neuroprosthesis in an individual with severe limb and vocal paralysis. Nat. Commun. 13, 6510 (2022).
[26]_Luo, S. et al. Stable Decoding from a Speech BCI Enables Control for an Individual with ALS without Recalibration for 3 Months. Adv. Sci. Weinh. Baden-Wurtt. Ger. e2304853 (2023) doi:10.1002/advs.202304853.
[27]_Angrick, M. et al. Online Speech Synthesis Using a Chronically Implanted Brain-Computer Interface in an Individual with ALS. http://medrxiv.org/lookup/doi/10.1101/2023.06.30.23291352 (2023) doi:10.1101/2023.06.30.23291352.
[28]_Schoenenberg, K., Raake, A. & Koeppe, J. Why are you so slow?-Misattribution of transmission delay to attributes of the conversation partner at the far-end. Int. J. Hum.-Comput. Stud. 72, 477-487 (2014).
[29]_Krauss, R. M. & Bricker, P. D. Effects of Transmission Delay and Access Delay on the Efficiency of Verbal Communication. J. Acoust. Soc. Am. 41, 286-292 (1967).
[30]_Brady, P. T. Effects of Transmission Delay on Conversational Behavior on Echo-Free Telephone Circuits. Bell Syst. Tech. J. 50, 115-134 (1971).
[31]_Sankaran, N., Moses, D., Chiong, W. & Chang, E. F. Recommendations for promoting user agency in the design of speech neuroprostheses. Front. Hum. Neurosci. 17, 1298129 (2023).
[32]_Mermelstein, P. Articulatory model for the study of speech production. J. Acoust. Soc. Am. 53, 1070-1082 (1973).
[33]_Willett, F. et al. Data for: A high-performance speech neuroprosthesis. 80440635830 bytes Dryad https://doi.org/10.5061/DRYAD.x₆₉P8CZPQ (2023).
[34]_Gaddy, D. Silent Speech EMG. Zenodo https://doi.org/10.5281/ZENODO.4064409 (2020).
[35]_Gaddy, D. & Klein, D. An Improved Model for Voicing Silent Speech. ArXiv210601933 Cs Eess (2021).
[36]_Crone, N. E., Miglioretti, D. L., Gordon, B. & Lesser, R. P. Functional mapping of human sensorimotor cortex with electrocorticographic spectral analysis. II. Event-related synchronization in the gamma band. Brain 121, 2301-2315 (1998).
[37]_Zhang, Q. et al. Transformer Transducer: A Streamable Speech Recognition Model with Transformer Encoders and RNN-T Loss. in ICASSP 2020-2020 IEEE International Conference on Acoustics, Speech and Signal Processing (ICASSP) 7829-7833 (IEEE, Barcelona, Spain, 2020). doi:10.1109/ICASSP40776.2020.9053896.
[38]_Rao, K., Sak, H. & Prabhavalkar, R. Exploring architectures, data and units for streaming end-to-end speech recognition with RNN-transducer. in 2017 IEEE Automatic Speech Recognition and Understanding Workshop (ASRU) 193-199 (IEEE, Okinawa, Japan, 2017). doi:10.1109/ASRU.2017.8268935.
[39]_Shi, Y. et al. Emformer: Efficient Memory Transformer Based Acoustic Model For Low Latency Streaming Speech Recognition. (2020) doi:10.48550/ARXIV.2010.10759.
[40]_He, Y. et al. Streaming End-to-end Speech Recognition For Mobile Devices. Preprint at http://arxiv.org/abs/1811.06621 (2018).
[41]_Graves, A. Sequence Transduction with Recurrent Neural Networks. (2012) doi:10.48550/ARXIV.1211.3711.
[42]_Hsu, W.-N. et al. HuBERT: Self-Supervised Speech Representation Learning by Masked Prediction of Hidden Units. Preprint at http://arxiv.org/abs/2106.07447 (2021).
[43]_Cho, C. J., Wu, P., Mohamed, A. & Anumanchipalli, G. K. Evidence of Vocal Tract Articulation in Self-Supervised Learning of Speech. in ICASSP 2023-2023 IEEE International Conference on Acoustics, Speech and Signal Processing (ICASSP) (IEEE, 2023). doi:10.1109/icassp49357.2023.10094711.
[44]_Casanova, E. et al. YourTTS: Towards Zero-Shot Multi-Speaker TTS and Zero-Shot Voice Conversion for Everyone. in Proceedings of the 39th International Conference on Machine Learning (eds. Chaudhuri, K. et al.) vol. 162 2709-2720 (PMLR, 2022).
[45]_Beukelman, D. R., Mirenda, P., & others. Augmentative and Alternative Communication. (Paul H. Brookes Baltimore, 1998).
[46]_Chiang, C.-H. et al. Development of a neural interface for high-definition, long-term recording in rodents and nonhuman primates. Sci. Transl. Med. 12, eaay4682 (2020).
[47]_Graves, A., Fernández, S., Gomez, F. & Schmidhuber, J. Connectionist temporal classification: labelling unsegmented sequence data with recurrent neural networks. in Proceedings of the 23rd international conference on Machine learning-ICML '06 369-376 (ACM Press, Pittsburgh, Pennsylvania, 2006). doi:10.1145/1143844.1143891.
[48]_Duraivel, S. et al. High-resolution neural recordings improve the accuracy of speech decoding. Nat. Commun. 14, 6938 (2023).
[49]_Mesgarani, N., Cheung, C., Johnson, K. & Chang, E. F. Phonetic Feature Encoding in Human Superior Temporal Gyrus. Science 343, 1006-1010 (2014).
[50]_Chang, E. F., Niziolek, C. A., Knight, R. T., Nagarajan, S. S. & Houde, J. F. Human cortical sensorimotor network underlying feedback control of vocal pitch. Proc. Natl. Acad. Sci. 110, 2653-2658 (2013).
[51]_Ozker, M. et al. Speech-induced suppression and vocal feedback sensitivity in human cortex. Preprint at https://doi.org/10.1101/2023.12.08.570736 (2023).
[52]_Guenther, F. H. et al. A Wireless Brain-Machine Interface for Real-Time Speech Synthesis. PLoS ONE 4, e8218 (2009).
[53]_Angrick, M. et al. Real-time synthesis of imagined speech processes from minimally invasive recordings of neural activity. Commun. Biol. 4, 1055 (2021).
[54]_Yamagishi, J., Veaux, C., King, S. & Renals, S. Speech synthesis technologies for individuals with vocal disabilities: Voice banking and reconstruction. Acoust. Sci. Technol. 33, 1-5 (2012).
[55]_Li, J. et al. Developing RNN-T Models Surpassing High-Performance Hybrid Models with Customization Capability. (2020) doi:10.48550/ARXIV.2007.15188.
[56]_Munteanu, C., Penn, G., Baecker, R., Toms, E. & James, D. Measuring the Acceptable Word Error Rate of Machine-Generated Webcast Transcripts. 4 (2006).
[57]_Fridriksson, J. et al. Speech entrainment enables patients with Broca's aphasia to produce fluent speech. Brain 135, 3815-3829 (2012).
[58]_Natraj, N. et al. Flexible regulation of representations on a drifting manifold enables long-term stable complex neuroprosthetic control. Preprint at https://doi.org/10.1101/2023.08.11.551770 (2023).
[59]_Sitaram, R. et al. Closed-loop brain training: the science of neurofeedback. Nat. Rev. Neurosci. 18, 86-100 (2017).
[60]_Sennrich, R., Haddow, B. & Birch, A. Neural Machine Translation of Rare Words with Subword Units. (2015) doi:10.48550/ARXIV.1508.07909.
[61]_Kudo, T. & Richardson, J. SentencePiece: A simple and language independent subword tokenizer and detokenizer for Neural Text Processing. (2018) doi:10.48550/ARXIV.1808.06226.
[62]_Lakhotia, K. et al. Generative Spoken Language Modeling from Raw Audio. (2021) doi:10.48550/ARXIV.2102.01192.
[63]_Wu, P., Watanabe, S., Goldstein, L., Black, A. W. & Anumanchipalli, G. K. Deep Speech Synthesis from Articulatory Representations. in Proc. Interspeech 2022 779-783 (2022). doi:10.21437/Interspeech.2022-10892.
[64]_Kong, J., Kim, J. & Bae, J. HiFi-GAN: Generative Adversarial Networks for Efficient and High Fidelity Speech Synthesis. (2020) doi:10.48550/ARXIV.2010.05646.
[65]_The LJ Speech Dataset. https://keithito.com/LJ-Speech-Dataset.
[66]_Kim, J., Kong, J. & Son, J. Conditional Variational Autoencoder with Adversarial Learning for End-to-End Text-to-Speech. (2021) doi:10.48550/ARXIV.2106.06103.
[67]_Willett, F. R., Avansino, D. T., Hochberg, L. R., Henderson, J. M. & Shenoy, K. V. High-performance brain-to-text communication via handwriting. Nature 593, 249-254 (2021).
[68]_Park, K. & Kim, J. g2pE. (2019).
[69]_Radford, A. et al. Robust Speech Recognition via Large-Scale Weak Supervision. Preprint at http://arxiv.org/abs/2212.04356 (2022).
[70]_Varshney, S., Farias, D., Brandman, D. M., Stavisky, S. D. & Miller, L. M. Using Automatic Speech Recognition to Measure the Intelligibility of Speech Synthesized From Brain Signals. in 2023 11th International IEEE/EMBS Conference on Neural Engineering (NER) I-6 (IEEE, Baltimore, MD, USA, 2023). doi:10.1109/NER52421.2023.10123751.
[71]_Jongseo Sohn, Nam Soo Kim, & Wonyong Sung. A statistical model-based voice activity detection. IEEE Signal Process. Lett. 6, 1-3 (1999).
[72]_Panayotov, V., Chen, G., Povey, D. & Khudanpur, S. Librispeech: an asr corpus based on public domain audio books in 2015 IEEE international conference on acoustics, speech and signal processing (ICASSP) (2015), 5206-5210.
[73]_Kingma, D. P. & Ba, J. Adam: A method for stochastic optimization. arXiv preprint arXiv:1412.6980 (2014).
[74]_Ito, K. & Johnson, L. The LJ Speech Dataset https://keithito.com/LJ-Speech576 Dataset/. 2017.
[75]_Kushal, L. et al. Generative spoken language modeling from raw audio. Transactions of the Association for Computational Linguistics (2021).
[76]_Morrison, M. et al. Chunked autoregressive GAN for conditional waveform synthesis. International Conference on Learning Representations (2022).
[77]_Goodfellow, I. et al. Generative adversarial networks. Communications of the ACM 63, 139-144 (2020).
[78]_Ren, Y. et al. Fastspeech 2: Fast and high-quality end-to-end text to speech. arXiv preprint arXiv: 2006.04558 (2020).
[79]_Gaddy, D. & Klein, D. Digital Voicing of Silent Speech 2020. https://arxiv.org/abs/2010.02960.
[80]_Gaddy, D. Voicing Silent Speech May 2022. http://www2.eecs.berkeley.edu/Pubs/TechRpts/2022/EECS-2022-68.html.

### Example 3: A High-Specificity Speech Neuroprosthesis With Shared Cortical Activity for Attempted Speech and Speech Perception

### 3.1. OVERVIEW

Speech neuroprostheses have the potential to restore naturalistic communication to people with paralysis by decoding intended speech from sensorimotor cortex (SMC) representations of their vocal-tract articulators. Speech neuroprostheses are usually evaluated in laboratory environments where attempted speech is isolated from other speech-related tasks, such as listening or reading. These functions, however, also engage neural populations on the SMC, raising the concern that they may interfere with speech decoding. To address this, a speech-decoding system was developed that maintained online performance and specificity to volitional speech attempts, regardless of whether two participants with vocal-tract paralysis were also participating in listening, reading, and non-speech mental imagery tasks. In offline analysis, though neural populations were observed that responded to attempted speech, listening, and reading, it was found that they leveraged different neural representations and their spectro-temporal response patterns were differentiable across tasks. Strikingly, these neural populations localized to the middle precentral gyrus and may have a distinct role in speech-motor planning. The results further understanding of shared activity on the SMC and demonstrate a maximally-reliable decoding framework for speech neuroprostheses.

An ECoG-based speech-decoder that maintains performance during perception, with most ‘shared’ production-perception activity in the middle precentral gyrus:

### 3.2. INTRODUCTION

Stroke or amyotrophic lateral sclerosis (ALS) can injure descending motor pathways in the brainstem, leading to paralysis and loss of the ability to move and articulate speech [12,13]. Despite this, recent work has demonstrated that neuroprostheses can restore lost function to people with paralysis, by recording brain activity and applying algorithms to decode this activity into intended movement [52,29,4,7,26,32,44]. These systems have predominantly leveraged persistent representations of low-level motor movements from the sensorimotor cortex (SMC). However, the SMC has been shown to not only contain representations of low level intended motor movements (e.g. movement parameters of the tongue or of one finger), but also multi-effector movements (e.g. representations of whole hand, whole body, or whole vocal-tract movements distinct from their single-effector components) as well as representations of visual and auditory stimuli. While evidence strongly suggests the brain leverages these multiplex representations, it is an open question whether single neural populations represent information the same for each function and what areas of the SMC are most likely to contain these representations.

These questions are particularly relevant and unexplored in the context of speech neuroprostheses. The nature and role of multiplexed representations, spanning reading, listening, and attempted speech, in the SMC are not yet well understood. Though studies of reading and listening have shown SMC activations, few to none have studied the overlap between these functions-particularly it is unknown if any studies have yet explored the overlap across all reading, listening, and speech production. One study on the overlap of speech and listening found that neural populations responding to both were in the SMC, but that they leveraged different representational codes for each. Some have hypothesized that SMC activations during perception occur at low-level motor control sites and extract perceived vocal-tract movements [35,36,25] while others have proposed that multisensory properties may signify a specific role for neural populations in motor planning [3,11].

In the context of speech neuroprostheses, multiplexed representations raise two important concerns: could reading or listening unintentionally engage a speech neuroprosthesis? Relatedly, could simultaneous reading or listening while speaking interfere with accurate decoding of speech attempts? Indeed, a key desire of potential speech neuroprosthesis users [24,34] is developing systems that are only activated by volitional speech attempts and maintain reliable decoding performance in real-world settings where the user may switch between engaging in various tasks, such as reading, listening, or accessing mental imagery, and attempting to speak. In fact, a recent study found that their speech-decoding system was falsely activated when participants were engaged in a listening task [43]. Thus, there is a need to both better understand the nature of multiplexed representations on the SMC and develop decoding frameworks that are robust to these representations and specific to the target motor output.

Here, the unique opportunity was used to investigate these questions, whether there are shared representations between all three functions and whether a speech neuroprosthesis can be designed to ensure specificity to attempted speech, in two participants with vocal-tract paralysis who had electrocorticography (ECoG) arrays chronically implanted over their SMC. A speech-decoding system was first developed that maintained reliable decoding performance and had no false positive activations during listening, reading, or non-speech mental imagery. This was accomplished by training speech detection and verification models on cortical activity from passive reading and listening, in addition to attempted speech. Offline, it was found that shared electrodes-those activated by attempted speech, reading, and listening-strongly localized to the middle precentral gyrus (midPrCG). Interestingly, these shared electrodes had distinct spectrotemporal response profiles for each task, allowing them to distinguish between attempted speech and perception. Perceptual content itself was not strongly decodable from shared electrodes; nor did the shared electrodes encode attempted vocal-tract movements. Instead, it was found that the shared electrodes may play a role in speech-motor planning.

### 3.3. RESULTS

3.3.1. Reading and Listening do not Interfere with Speech Decoding

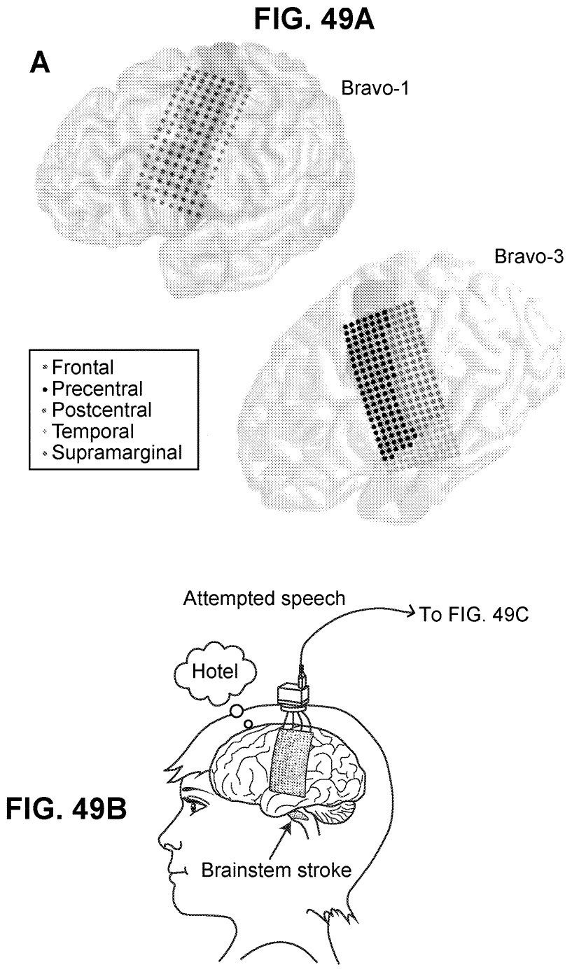

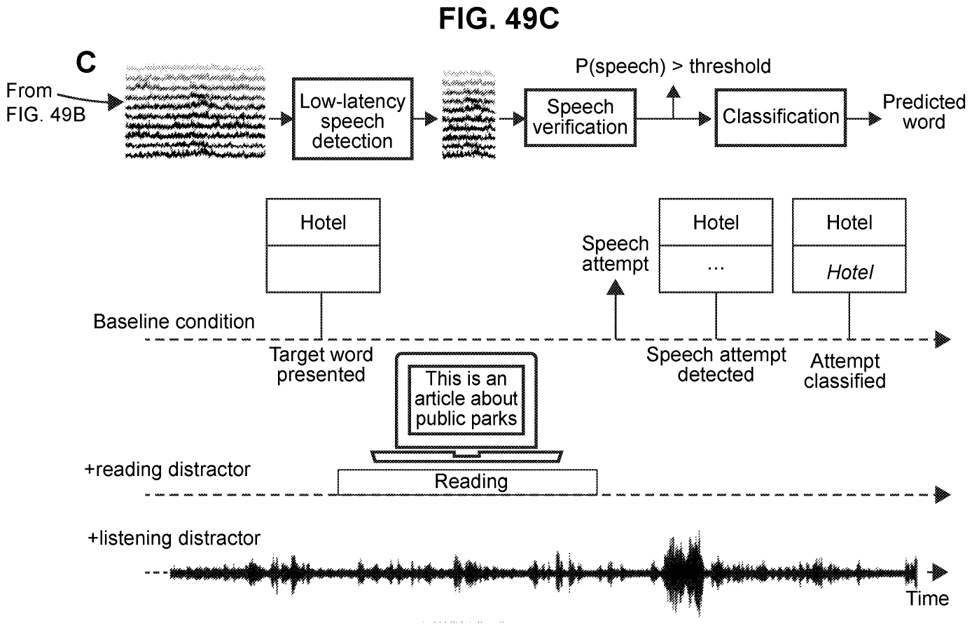

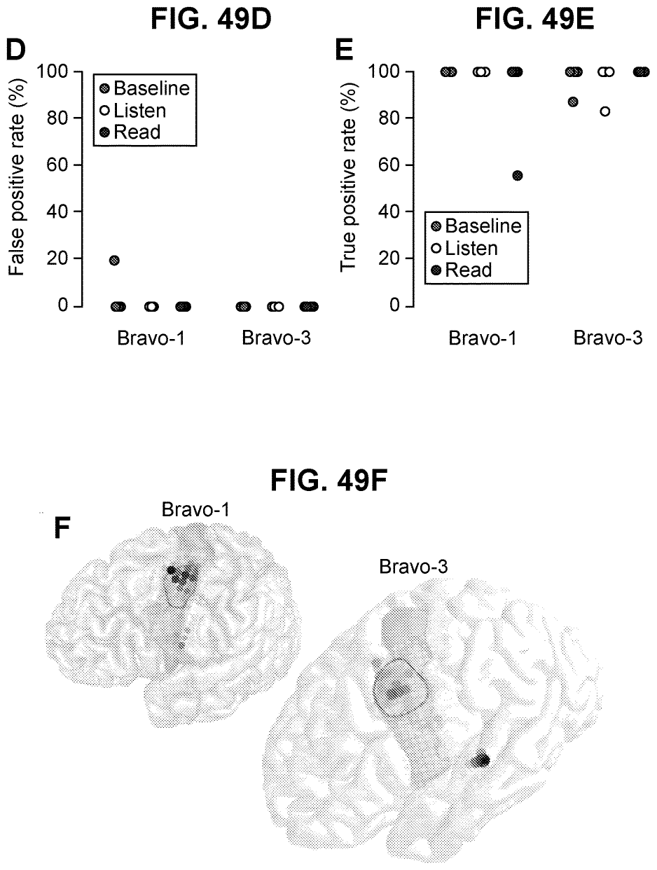

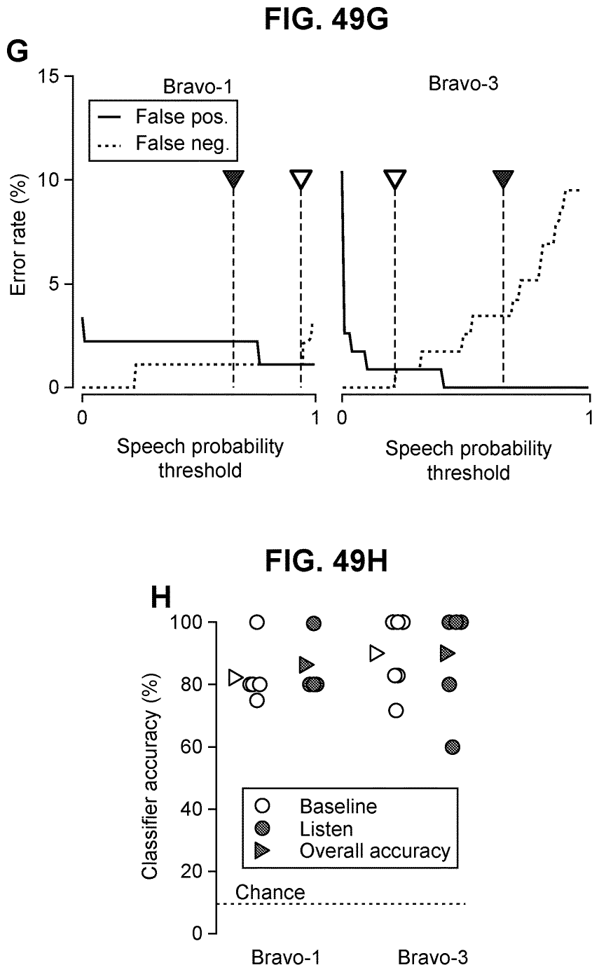

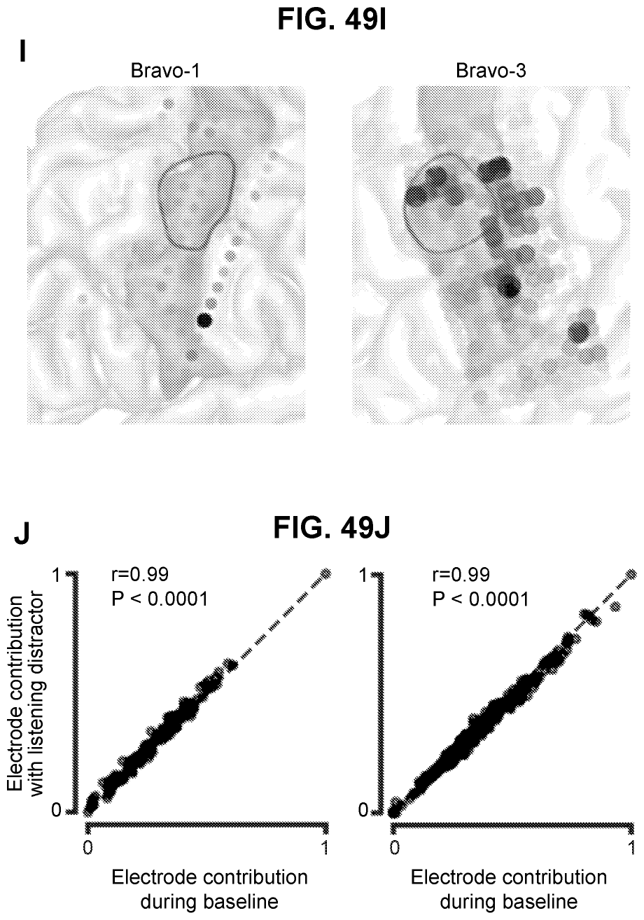

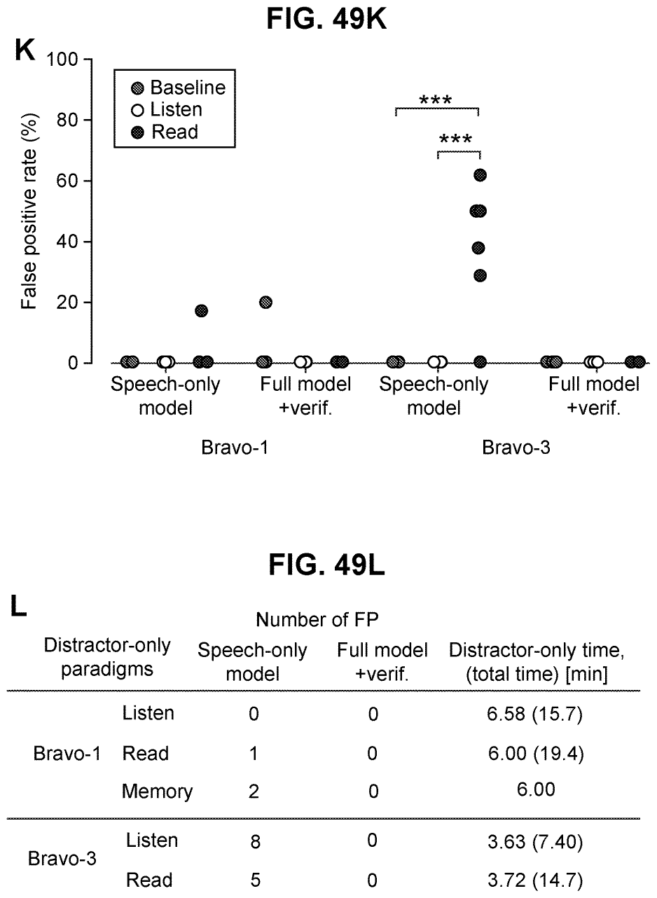

A speech-decoding system was developed that was robust to reading and listening in two participants (ClinicalTrials.gov; NCT03698149) with vocal-tract paralysis and anarthria, referred to as Bravo-1 and Bravo-3. In both participants, cortical activity was measured with electrocorticography (ECoG) arrays covering frontal, precentral, postcentral, and temporal cortex (FIG. 49A). Bravo-1 attempted to speak, producing audible grunts that were unintelligible. Bravo-3 silently attempted to speak, making uncoordinated vocal-tract movements with no produced sound. To decode cortical activity into intended speech while minimizing interference from perceptual functions, a system consisting of three key components was designed (FIG. 49B). While each participant attempted to say a target word from a predefined 10-word vocabulary, the high-gamma activity (HGA; 70-150 Hz) and low-frequency signals (LFS; 0.3-17 Hz; see Methods) were streamed from each electrode to a speech-detection model trained to identify volitional speech attempts at low latencies (approximately 0.5s, see Methods). Next, candidate speech events from the detector were then passed to a speech-verification model, which was trained to disambiguate attempted speech from reading and listening, acting as an additional mechanism to minimize false positives. Finally, if the probability of attempted speech exceeded an acceptable threshold, the speech event was passed to a classifier, which outputs the most likely word based on the neural features.

To test the robustness of the system to perceptual distractors, three online conditions were evaluated under, each consisting of three 10-trial blocks. Here, a distractor was defined as a non-attempted speech task that may falsely activate or affect accuracy of the speech-decoding system. In each block, a target word was first presented on the monitor and the participant attempted to speak the word at their own pace (FIG. 49C). This attempt was detected, verified, and classified by the system, after which the task moved to the next trial. In the baseline condition, no perceptual distractor was present during the block. In the reading distractor condition, the participant read sections of an article on a separate laptop before making a speech attempt (Video 1). Finally, in the listening distractor condition, a podcast was playing for the duration of the block, and the participant listened to the podcast before making a speech attempt (Video 2).

An ideal system would have a zero false positive rate (FPR), defined as the proportion of detected events that are not associated with any speech attempt, and maximize the true positive rate (TPR), defined as the proportion of true speech attempts that are detected. The FPR and TPR were measured during online evaluations during the baseline, listening, and reading distractor conditions. For Bravo-1, FPR was not significantly increased (FIG. 49D, P >0.01, Fisher exact test), with only one false positive detection, which was associated with a laugh, for an overall FPR of 1.15%. Of note, Bravo-1 had several non-speech vocalizations, some due to his type of paralysis, which can cause involuntary laughs. However, of the 31 non-speech vocalizations that occurred during decoding, only one resulted in the aforementioned false positive. For Bravo-3, FPR was unaffected and remained at 0% for all distractor conditions. For both participants, it was found that the presence of listening or reading distractors did not significantly decrease the TPR (FIG. 49E, P>0.01, Fisher exact test), which was 95.6% and 98.0% overall for Bravo-1 and Bravo-3, respectively. Neural activity in the midPrCG contributed most for detecting speech attempts in Bravo-1. For Bravo-3, both the midPrCG and a small cluster in the posterior superior temporal gyrus (pSTG) were most important for detecting speech events (FIG. 49F).

When using the full system, both the speech detector and the speech-verification model affect the FPR and TPR. For the speech-verification model, the choice of a probability threshold, above which to verify a detected event as attempted speech, affects whether detected events associated with a true speech attempt are mistakenly rejected (false negative) or whether detected events that are not associated with a true speech attempt and mistakenly accepted (false positive). For all the detected events during the online evaluation, the FPR and false negative rate (FNR) were measured as the speech probability threshold was moved between 0 and 1. For both participants, it was observed that lower thresholds, which are more generous in accepting events, result in low FNR but increase FPR, as expected (FIG. 49G). The reverse is true for more conservative, higher thresholds. Based on offline evaluations, the threshold was set to 0.65 during online evaluations for both participants (filled triangle, FIG. 49G). While this threshold performed well during online evaluations, adjusting the threshold would have further reduced false positives in Bravo-1 and reduced false negatives in Bravo-3 (unfilled triangle, FIG. 49G).

To test whether perceptual distractors may affect the system's decoding accuracy, classification accuracy was compared between the baseline condition and the listening condition, where a podcast was playing during the participants' speech attempts. No significant differences in classification accuracy were found between the two conditions (P>0.01, Fisher exact test, FIG. 49H). In contrast to the speech detector, electrodes in the vSMC centered along the central sulcus were of greatest importance to the classifier (FIG. 49I). The classifier placed importance on the same electrodes during the baseline and listening condition (FIG. 49J; Pearson correlation, r=0.99 and P<0.0001 for both Bravo-1 and Bravo-3).

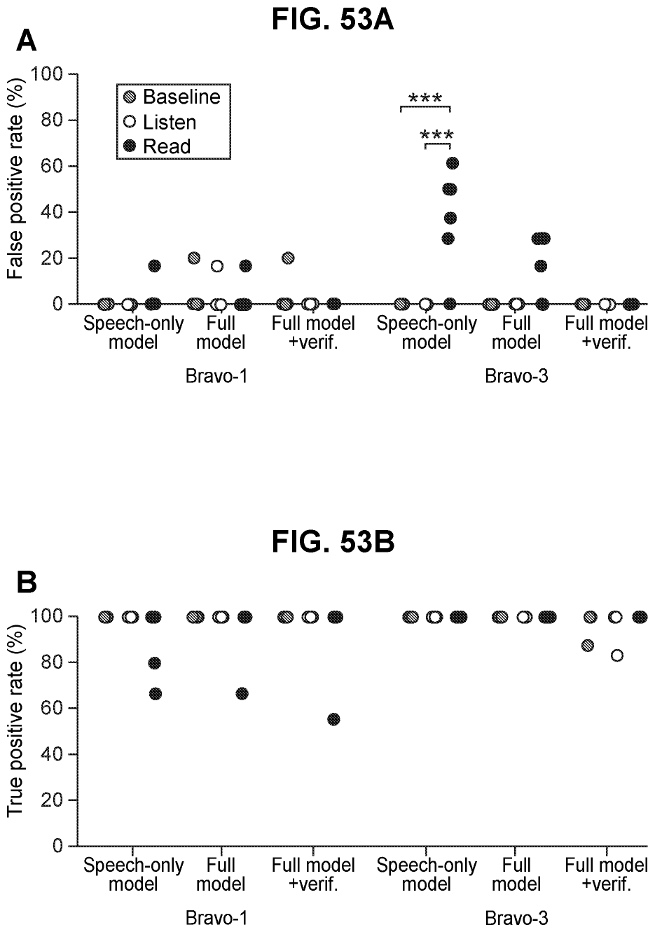

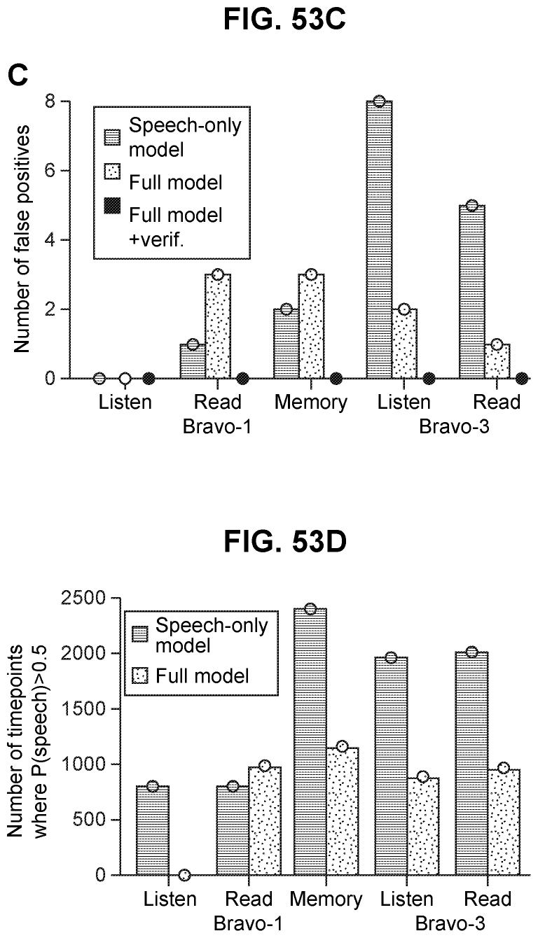

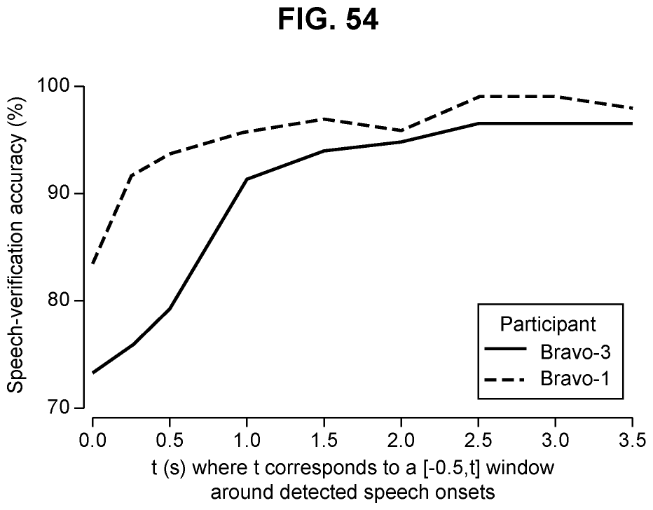

For the full system, two primary design choices were made to reduce FPR. First, examples of listening and reading were included in training the speech detector (for Bravo-1, only listening was added, see Methods) to increase the specificity of the model to attempted-speech events. Second, the speech-verification model was included. While the speech detector works at lower latencies, the speech-verification model receives a larger window relative to the onset of each detected event (4 s for Bravo-1 and Bravo-3, see Methods), allowing use of longer context lengths to disambiguate attempted speech from reading and listening. Offline, system ablations were performed to determine the effects of both these design choices on TPR and FPR. It was found that while removing just the speech-verification model increased the absolute number of false positives, it did not significantly increase the FPR (FIG. 53B) for either participant. However, removing the speech-verification model and using a speech detector trained only on speech attempts (and not reading or listening examples), significantly increased the FPR during the reading distractor for Bravo-3 (P<0.0001, Fisher exact test, FIG. 49K, FIG. 53B) but not during the listening distractor. For both participants, neither ablation significantly affected the TPR, as expected (FIG. 53A). Given that low-latency decoding may be desirable [23], speech-verification model performance was simulated as a function of the length of the window relative to detected onset used for attempted-speech verification. In Bravo-1, roughly 95% verification accuracy was achieved as quickly as a second after the detected onset, but in Bravo-3, performance rose more slowly, reaching roughly 95% at around 2 seconds (FIG. 54). This discrepancy may stem from stronger evoked activity, given array coverage, for reading and listening in Bravo-3, which were later explore in more detail. Though these hypothetical latencies (relative to the onsets) are longer than what is observed with the speech detection model (see Methods), the added safeguard can be beneficial.

To further test the robustness of this system to false positives, a handful of longer trials (roughly 2 minutes each) were also recorded while the participants were asked not to make any speech attempts and only listen, read, and, for Bravo-1, perform a mental imagery task where he was instructed to think of various memories via autobiographical questions (Video 3). Using the full system online, zero false positives were observed for both participants for a total duration of about 18.6 and 7.35 minutes of perceptual distractors for Bravo-1 and Bravo-3, respectively (FIG. 49L). When combined with the trials where participants made speech attempts during distractors, this totals to zero false positives over 41.1 and 22.1 minutes for Bravo-1 and Bravo-3, respectively (FIG. 49L). Additionally, in Bravo-1 the system totaled zero false positives over 6 minutes of a mental imagery task, demonstrating robustness to a form of internal thoughts. Offline, the same system ablations were performed for these longer evaluation blocks, similarly finding that false positives generally increase as precautions are removed (FIG. 53C). Of note, although system ablations had no effect on the FPR during listening for Bravo-1, the inclusion of listening examples during training decreased the amount of time points that have a predicted speech probability above 0.5 (the probability threshold for the speech detector) (FIG. 53D). Overall, these results show that the decoding system is robust to perceptual distractors, and that the inclusion of distractors in model training allows the system to directly model differences in these functions, thereby reducing false positives.

FIGS. 49A-49L: Reading and listening do not interfere with speech decoding. FIG. 49A: Cortical activity is recorded using chronically implanted ECoG grids in two participants. Bravo-1 has a 128 channel 4 mm spaced grid while Bravo-3 has a denser 253 channel 3 mm spaced grid. Electrodes are colored according to anatomical region and the precentral gyrus is shaded. FIG. 49B: A speech decoding system was designed with 3 main components—a low-latency neural speech detector, a speech-verification classifier, and a 10-word classifier. Filtered neural activity is processed by the speech-detector in real time, which generates discrete events associated with the participant's attempted speech. Neural activity associated with this event is first passed to the speech verification classifier, which acts as an extra precaution, to ensure that the event corresponds to attempted speech. If so, the event is finally passed to the 10-word classifier which generates the predicted word. FIG. 49C: The decoding framework was tested in a variety of settings. First, in a “baseline” setting where target words are presented on the screen and the participant attempts them at their leisure. Next, in a “reading” distractor setting where the participant is instructed to alternate between reading articles presented on a separate screen and attempting the target word. Finally, in a “listening” distractor setting where a podcast is continuously playing in the background and the participant is instructed to alternate between attending to the podcast and completing the task. FIG. 49D: The false positive rate (FPR) of the full speech decoding system (with the speech-verification classifier). FIG. 49E: The true positive rate (TPR) of the full speech decoding system (with the speech-verification classifier) during baseline, listening, and reading blocks. FIG. 49F: Electrode contributions for the speech-detection model for Bravo-1 and Bravo-3. The midPrCG is outlined in black. FIG. 49G: The false positive and negative rates as a function of the probability threshold used in the speech verification classifier. Vertical green lines with filled markers note the threshold used during real-time testing (0.65) while unfilled markers note the optimal threshold that minimizes both error rates (calculated offline). FIG. 49H: Accuracy of the 10-word classifier during baseline and listening blocks across 6 pseudo-blocks. Slightly offset triangle markers note the average accuracy across all trials, which was 82.1% without and 86.2% with the listening distractor for Bravo-1, and 90.0% for both for Bravo-3 (triangle markers, FIG. 49H). FIG. 49I: Electrode contributions for the 10-word classifier for Bravo-1 and Bravo-3. FIG. 49J: Scatter plot comparing electrode contributions for the 10-word classifier during online evaluations with the listening distractor versus without. Pearson correlation coefficient is noted. FIG. 49K: FPR of the decoding system when using the full system and a speech-detection model only trained on speech (and not reading or listening), across 6 pseudo-blocks. ***P<0.0001. FIG. 49L: The number of false positives using the full system and a speech-only speech detection model during long periods of listening and reading (and memory reflection during an autobiographical interview in Bravo-1, see Methods). In these blocks, the system was tested during prolonged periods of reading and listening with no attempted speech targets. The amount of time where the decoding system was active, in which false positives could occur, is also noted. In parentheses, the total amount of time with any one of the distractors (the combination of distractor-only blocks with the main task that includes speech attempts) is noted. When using the full system, there were 0 false positives for the total time.

FIGS. 53A-53D: Effect of speech decoding system ablations on false positive and negative performance. FIG. 53A: The false positive rate (FPR) of the system with only a speech-detection model trained only on attempted speech (no reading or listening data), the full model (trained with reading and listening data but without the speech-verification classifier), and the full speech decoding system during baseline, listening, and reading blocks. FPR generally increases as system protections are removed (e.g. not using the speech-verification model and not giving the speech-detection model examples of listening and reading to dissociate from attempted speech), however, they are only significantly increased for the speech-only model during a reading distractor with Bravo-3 (***P<0.0001, Fisher exact test with 9-way Holm-Bonferroni correction). FIG. 53B: The true positive rate (TPR) for each of the system ablations in FIG. 53A during baseline, listening, and reading blocks. TPR is not significantly different for any of the distractors or system ablations, for either participant. FIGS. 53A-53B are extensions of panels FIGS. 53D-53E in FIG. 49. FIG. 53C: The absolute number of false positives for each of the system ablations during “long” distractor blocks of listening and reading, where there were no speech attempts and only the distractor. For Bravo-1, this also included memory reflection during an autobiographical interview block (see Methods). For Bravo-1, who had much less of a pronounced overlap between reading, listening, and attempted speaking neural responses, a speech-only model outperformed a model that included examples of listening. Using the full system still resulted in the best performance. For Bravo-3, including reading and listening in the speech-detection model and then adding the speech-verification model both decreased the number of false positives. FIG. 53D: The number of time points (at 200 Hz) that had a speech probability greater than the probability threshold of 0.5 for each of the system ablations and “long” distractor blocks. These time points are not all consecutive and can reflect brief spikes in the probability that do not result in false positive events. For reference, 2500 time points corresponds to a cumulative 12.5 seconds of time points with a speech probability above 0.5, while 500 corresponds to 2.5 seconds. Even though including reading and listening in the speech-detection model (“Speech-only model” versus “Full model”) did not have an effect or resulted in slightly more false positive events (as shown in FIG. 53C, with no effect on listening, 2 more events during reading and 1 more event during memory reflection), the full model resulted in few time points with high speech probability. This suggests that while it was not enough to decrease the number of events, the addition of reading and listening examples still generally improves the accuracy of the continuously predicted probabilities.

FIG. 54: Speech-verification model performance for different time windows around detected events. Shown is the accuracy of the speech-verification model in accepting/rejecting detected speech-events using various time windows. Performance is shown using time windows spanning [−0.5, t] around a detected event, where t is iteratively increased to 3.5s.

### 3.3.2. Shared and Distinct Cortical Activations for Reading, Listening, and Attempted Speech

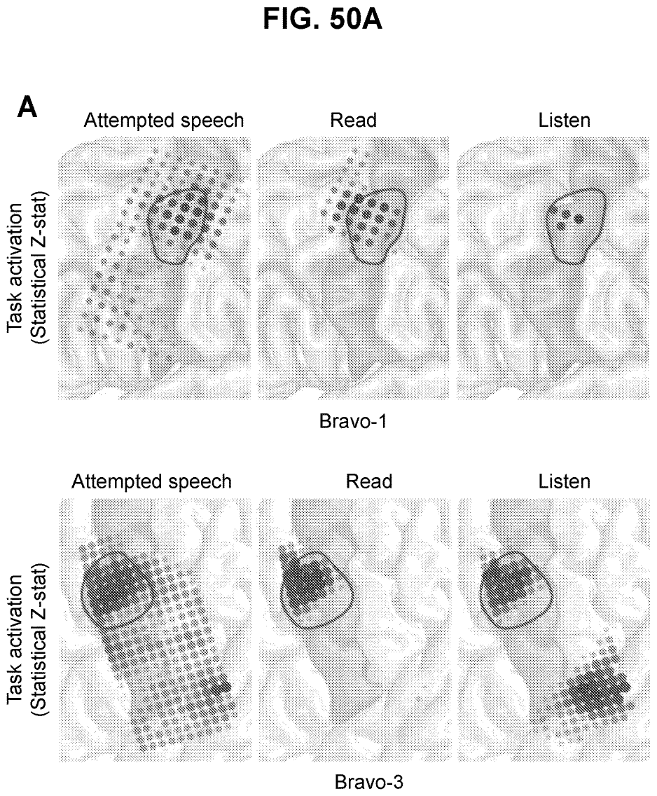

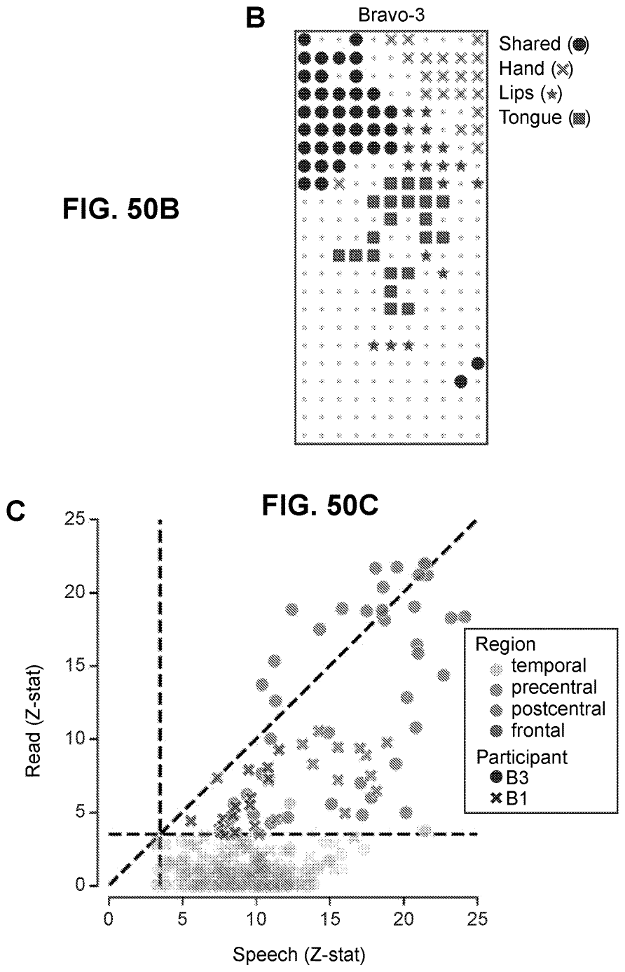

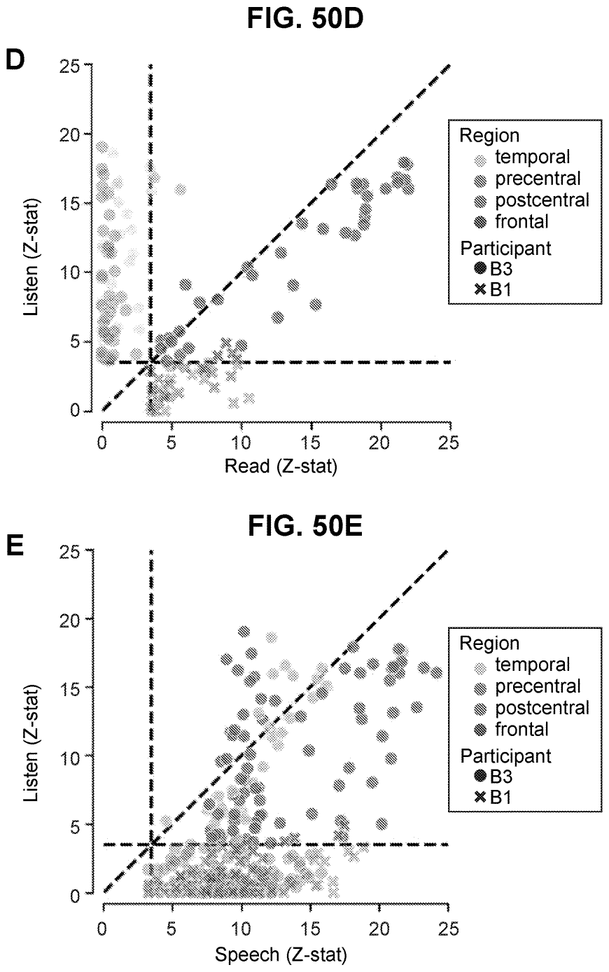

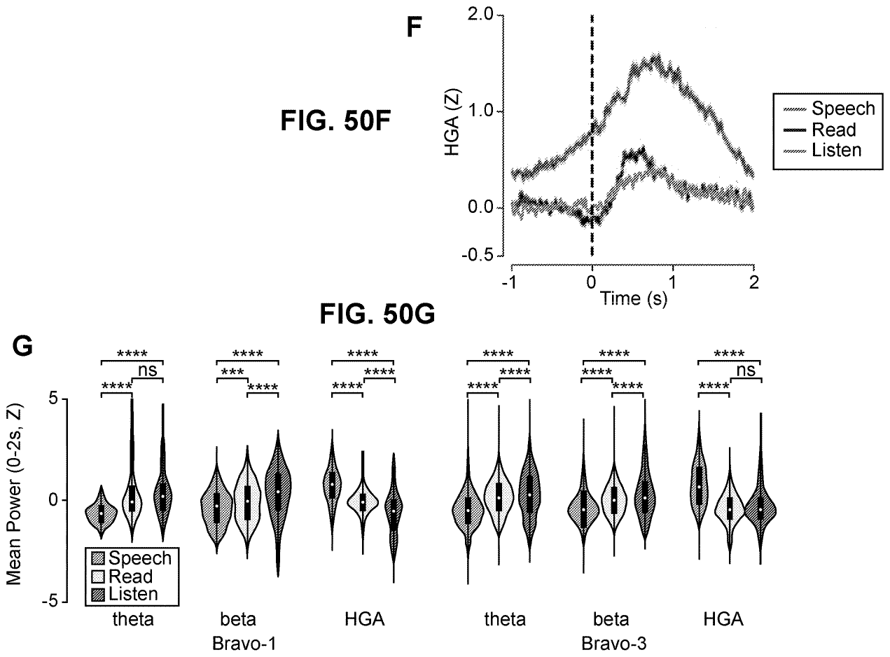

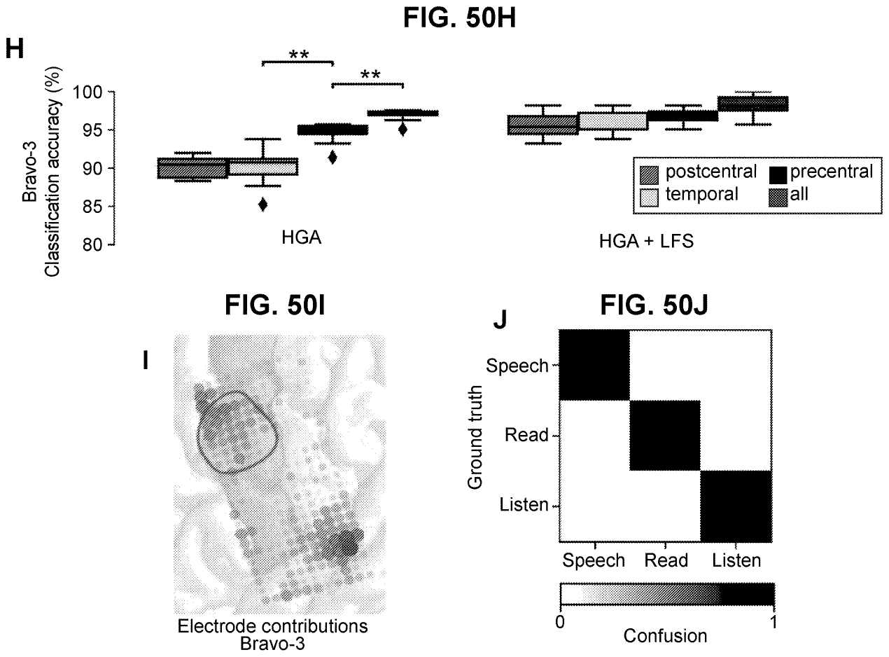

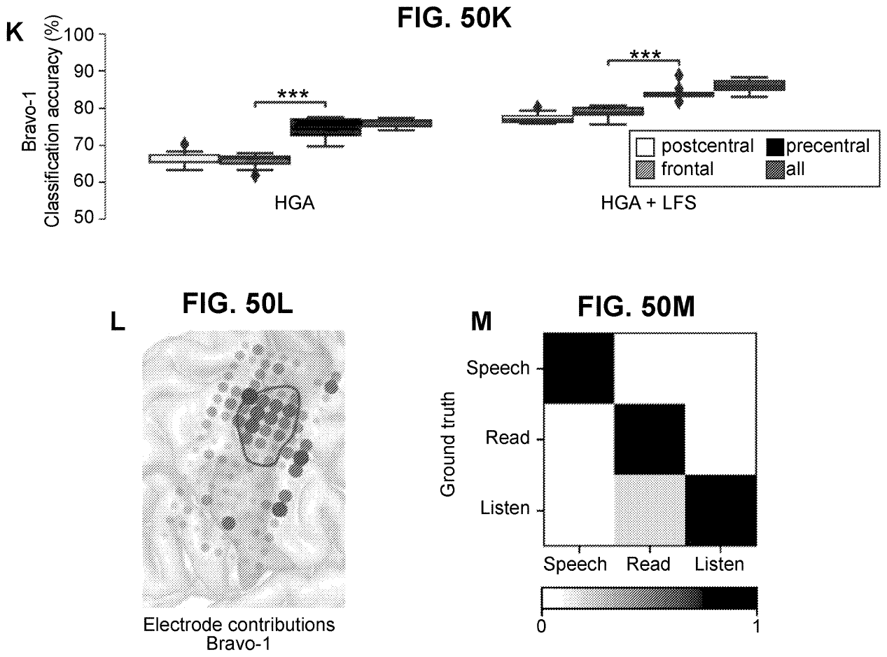

To further characterize the neural features and cortical regions that allow for disambiguation of reading, listening, and attempted speech, several offline analyses were performed. For each electrode, whether HGA was significantly increased during each of the three tasks, relative to pre-trial resting activity (FIG. 50A-C; two-sided Wilcoxon rank-sum tests with 128-way (Bravo-1) or 253-way (Bravo-3) Holm-Bonferroni correction) was tested. Across the two participants, the midPrCG and select electrodes in the STG were most strongly activated across reading, listening, and attempted speech (herein referred to as “shared” electrodes; FIG. 50A). Interestingly, shared electrodes did not overlap with electrodes found to encode attempted vocal-tract (speech articulatory) movements (FIG. 50B), localizing to the midPrCG and pSTG. Across both participants, there were no neural populations that were responsive during reading and not responsive during attempted speech (FIG. 50C). A majority of reading electrodes also had elevated activity during listening (FIG. 50D). Listening responses were also found in the temporal lobe and overlapped strongly with attempted-speech but less so with reading (FIG. 50D-E).

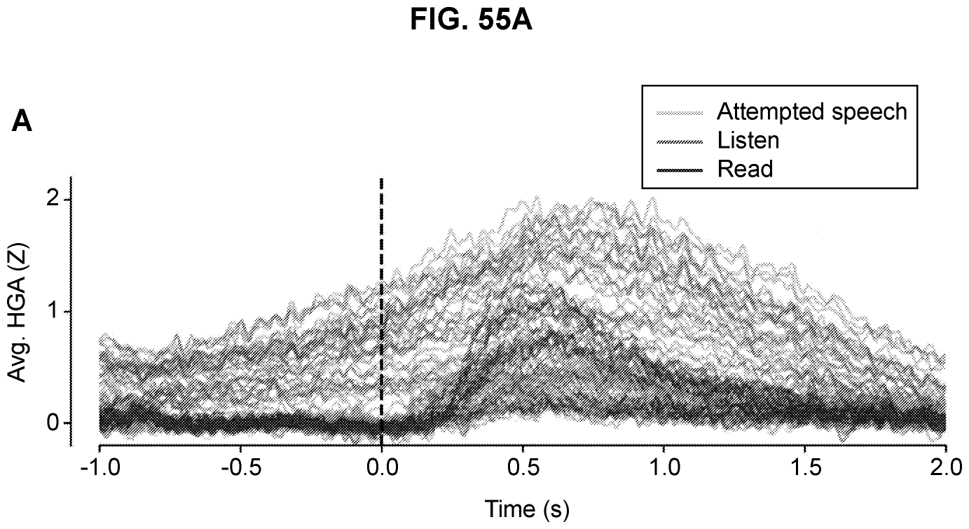

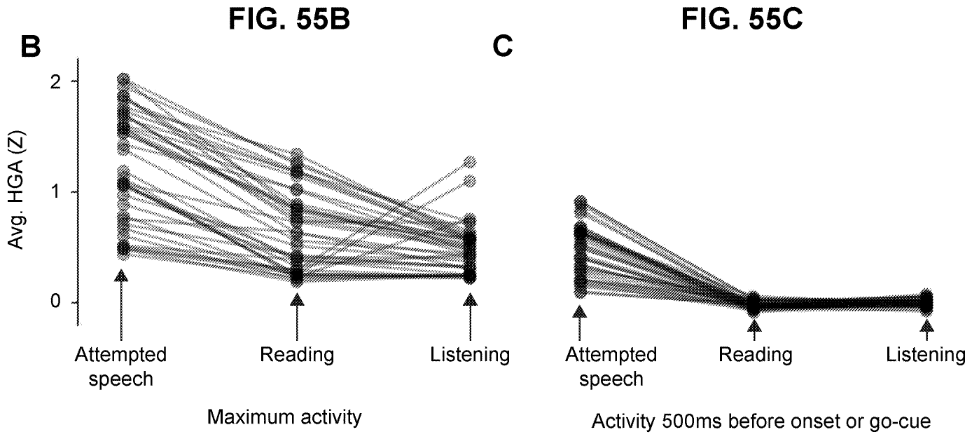

It was next asked if shared electrodes had different spectrotemporal responses for each function. It was found that, at shared electrodes, attempted speech generally evoked the highest maximum HGA followed by reading and listening (FIG. 50F, FIG. 55). Shared electrodes also had increased HGA before the go-cue, in contrast to reading and listening (FIG. 50F, FIG. 55). Shared electrodes further differed in their low-frequency content; attempted speech had significantly stronger suppression in theta-(5-12 Hz) and beta-band (20-30 Hz) power relative to reading and listening (FIG. 50G; two-sided Wilcoxon rank-sum tests with 12-way Holm-Bonferroni correction). This aligns with literature showing that there is low-frequency desynchronization during intact motor movements [8,41] and synchronized low-frequency signals during perceived speech [21,31]. Thus, despite shared electrodes having significant HGA for each function, spectro-temporal response profiles for each are distinct.

To investigate the contribution of these broad anatomical and spectral differences to differentiating between reading, listening, and attempted speech, offline training of the speech-verification model was limited to these different anatomical regions. In Bravo-3, discriminability between reading, listening, and attempted speech is strong across anatomical areas, with the best performance from the precentral gyrus (FIG. 50H; two-sided Wilcoxon rank-sum tests with 12-way Holm-Bonferroni correction). In line with spectral analysis (FIG. 50G), incorporation of low frequency signals (LFS) further improved classification performance over HGA alone (FIG. 50H). Interestingly, electrodes that contribute most to performance are in the midPrCG and STG, providing further evidence that, despite shared activations, underlying spectrotemporal profiles may be strongly differentiable for each task (FIG. 501). While all three functions are differentiable, reading and listening are more likely to be confused for one another than for attempted speech (FIG. 50J). In Bravo-1, performance was highest using electrodes from the precentral gyrus and adding LFS increased performance (FIG. 50K; two-sided Wilcoxon rank-sum tests with Holm-Bonferroni correction for multiple comparisons). Similar to Bravo-3, electrodes driving performance were in the midPrCG (FIG. 50L). Although overall speech-verification classification accuracy was lower in Bravo-1, this was due to confusability between listening and reading with preserved separation of attempted speech from reading or listening (FIG. 50M).

FIGS. 50A-50M: Shared and distinct cortical activations for reading, listening, and attempted speech. FIG. 50A: Electrode heat maps for responsiveness in each task measured by non-parametric tests of pre-trial vs post go-cue mean high-gamma amplitude (HGA). Only electrodes with significant task modulation (P<0.05; two-sided Wilcoxon rank-sum tests with 128-way (Bravo-1) and 253-way (Bravo-3) Holm-Bonferroni correction for multiple comparisons) are shown. Results are shown for Bravo-3 (right) and Bravo-1 (left). Black region outline indicates the midPrCG and blue shading the precentral gyrus. FIG. 50B: In Bravo-3, electrodes that have task modulation (P<0.05; two-sided Wilcoxon rank-sum tests with 253-way Holm-Bonferroni correction) for reading, listening, and attempted speech are visualized alongside electrodes that encode movements of the vocal-tract articulators during continuous speech and hand. FIGS. 50C-50E: Two-sided Wilcoxon rank-sum test statistics (as in FIG. 50A) for each electrode are plotted for reading and speech (FIG. 50C) speech and listening (FIG. 50D), and listening and reading (FIG. 50E). Vertical lines represent rough thresholds for statistical significance (as in FIG. 50A). The unity line (y=x) is also shown. FIG. 50F: Example evoked response potentials (ERPs) for an electrode in Bravo-3 that has significant task modulation across attempted speech, reading, and listening. Error bars represent standard error of the mean. FIG. 50G: Shown is the mean high-gamma amplitude, theta power, and beta power during reading, listening, and attempted speech across electrodes with significant task modulation (as in FIG. 50A) for listening, reading, and attempted speech (****P<0.0001; two-sided Wilcoxon rank-sum tests with 6-way Holm-Bonferroni correction for multiple comparisons). FIG. 50H: Shown is classification accuracy for the speech-verification model, trained on specific regions and feature streams in Bravo-3. Distributions are over 10 non-overlapping cross validation folds (**P<0.01; Two-sided Wilcoxon rank-sum tests with 6-way Holm-Bonferroni correction for multiple comparisons). Statistical results are shown for adjacent distributions after sorting by median classification accuracy. FIG. 501: Shown are electrode contributions to the full speech-verification model (all, HGA+LFS) in Bravo-3. FIG. 50J: Shown is a confusion matrix for predictions from the full speech-verification model in Bravo-3. FIGS. 50K-50M: The same as (FIGS. 50H-50J) for Bravo-1. In (FIGS. 501 and 50L) the black region outline indicates the midPrCG and blue shading the precentral gyrus.

FIGS. 55A-55C: Temporal dynamics of shared activity during attempted speech, listening, and reading. FIG. 55A: Shown are mean evoked response potentials (ERPs) for attempted-speech, listening, and reading. ERPs are shown across all tri-function electrodes. FIG. 55B: For each tri-function electrode, the maximum HGA evoked by attempted speech, reading, and listening is shown. FIG. 55C: For each tri-function electrode, the evoked HGA 500 ms before the onset/go-cue for attempted speech, reading, and listening is shown.

### 3.3.3. Shared and Distinct Cortical Activations for Reading, Listening, and Attempted Speech

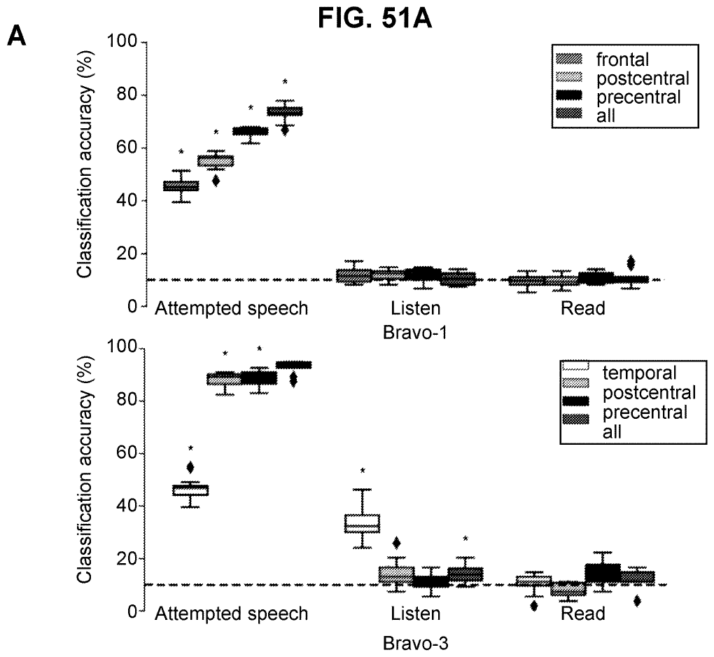

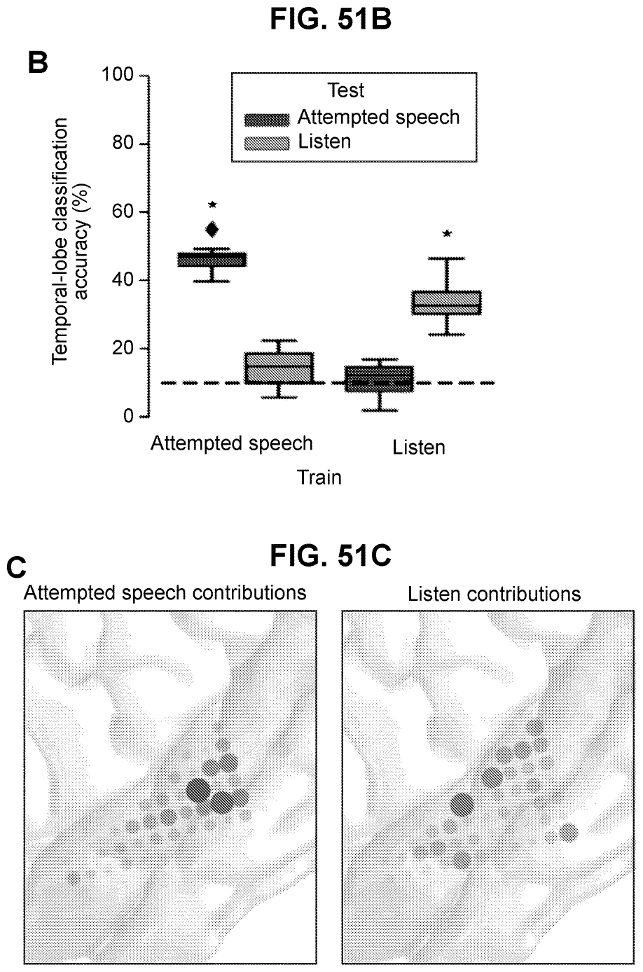

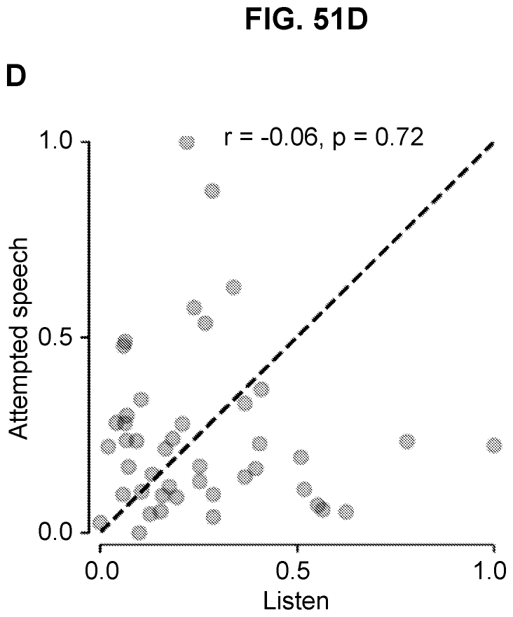

Whether the representations for attempted speech, listening, and reading were decodable and, if so, shared across cortical regions was investigated next. The 10-word classifiers were trained on cortical-activity from each anatomical region during attempted-speech, reading, and listening (FIG. 50A). While electrodes were activated by reading (FIG. 50A), reading content was not decodable from any anatomical region covered by the participants' ECoG arrays (two-sided Wilcoxon signed-rank test with 12-way Holm-Bonferroni correction). Listening was only decodable from the temporal-lobe in Bravo-3. Interestingly, attempted speech was decodable across all regions, including the temporal-lobe, despite Bravo-3's attempted speech being silent (FIG. 51A). Given that the temporal lobe in Bravo-3 was the only region where multiple functions could be decoded, it was asked whether it leveraged a distinct or shared representation for attempted speech and listening. 10-word classifiers trained on attempted speech (listening) were used to predict on listening (attempted speech) trials. Low generalizability of the models across functions was found. Additionally, contribution values for decoding attempted speech versus for decoding listening from the same electrodes were not significantly correlated (P=0.74, Spearman correlation permutation test; FIG. 51C, D). Together, these results suggest that despite neural populations having activations for both attempted speech and listening, they are not leveraging identical representations for each function. This provides further evidence that speech-decoding models are unlikely to read out perceived content if an appropriate speech detection and verification system is used.

FIGS. 51A-51D: Distinct representations for attempted speech, listening, and reading on speech cortex. FIG. 51A: Shown is classification accuracy by anatomical region on the isolated-word set during reading, listening, and attempted speech. Statistical annotations reflect above-chance performance (P<0.05; two-sided Wilcoxon signed-rank test with 12-way Holm-Bonferroni correction for multiple comparisons) FIG. 51B: For the Bravo-3 temporal-lobe attempted speech and listening classification models, accuracy is shown for evaluating models across tasks. Statistical annotations reflect above-chance performance (P<0.05; two-sided Wilcoxon signed-rank test with 4-way Holm-Bonferroni correction for multiple comparisons). FIGS. 51A-51B: distributions are over 10 non-overlapping cross validation folds of the data. FIG. 51C: Shown are electrode contributions for Bravo-3 temporal-lobe attempted speech and listening classification models. Yellow region shading indicates the superior temporal gyrus. FIG. 51D: Scatterplot of electrode contributions from FIG. 51C across temporal-lobe electrodes (r=−0.06 and P=0.72; Non-parametric Spearman correlation and permutation test).

### 3.3.4. a Role for Shared Electrodes in Speech-Motor Planning

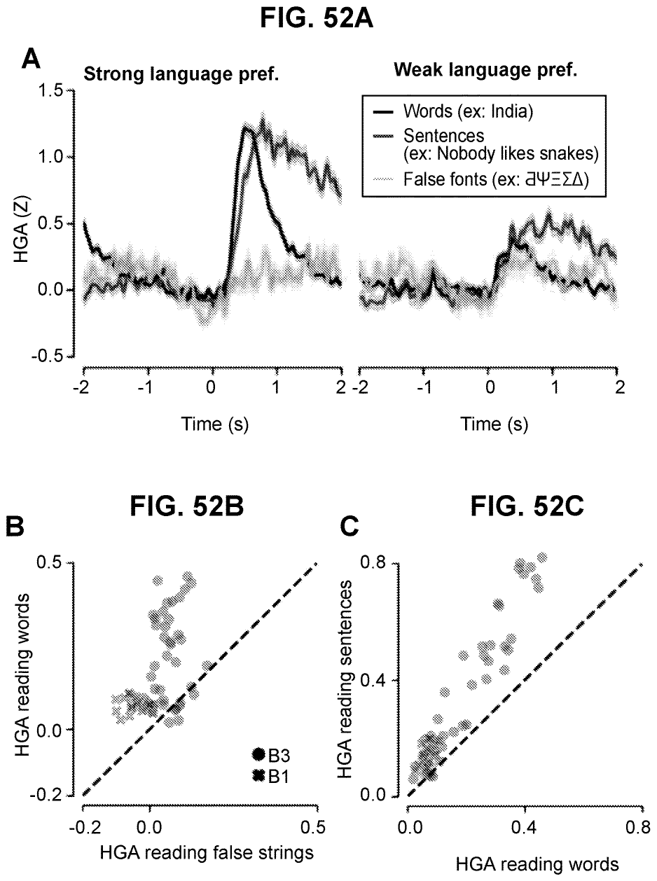

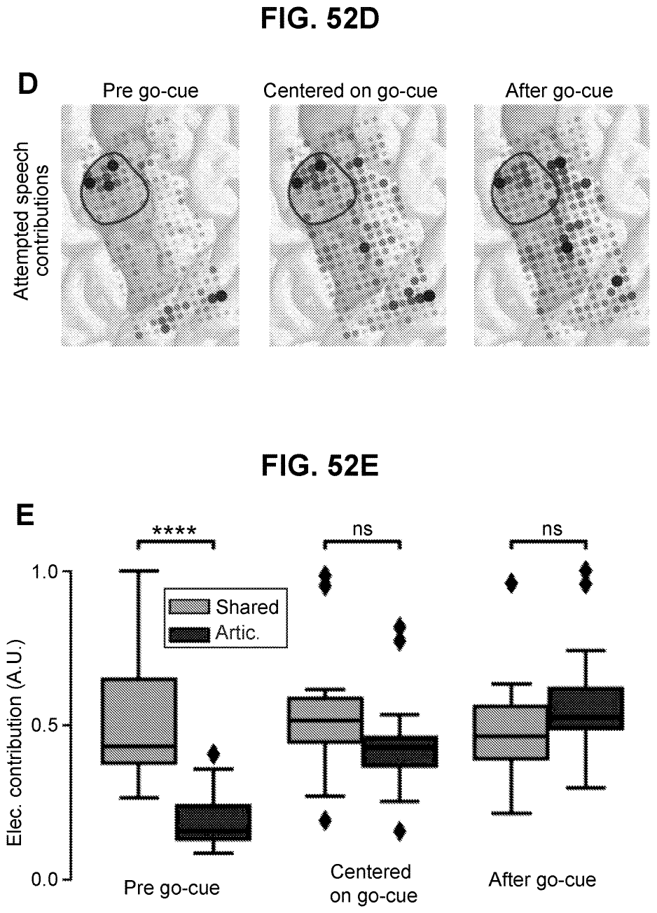

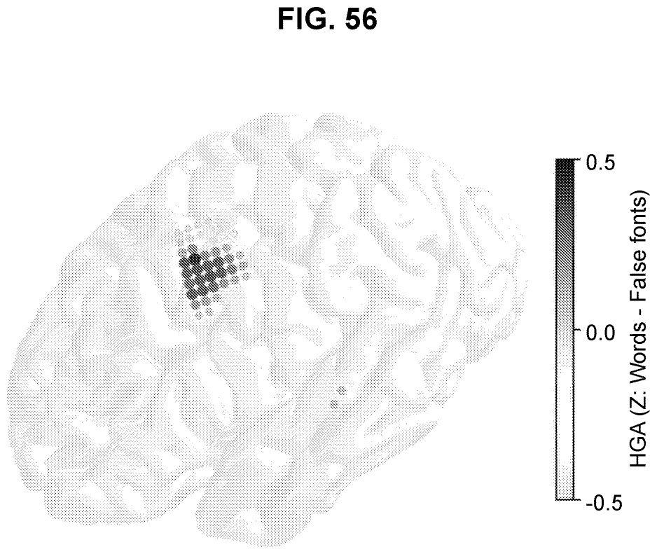

Despite reading activity not being decodable, it was assessed whether they were stronger for linguistic content, potentially indicating a meaningful role in speech, or a product of visual changes on the screen. To test this, the participants were presented with sentences and with false fonts, such as “δ [symbol] . [54] to read, in addition to the isolated words. Across both participants, evoked HGA was stronger for reading the isolated words versus false fonts (two-sided Wilcoxon signed-rank test; FIG. 52A-52B). The few electrodes with comparable evoked HGA were located near to the frontal eye fields [14] in Bravo-3 (FIG. 52A, FIG. 56). Over a 2-second window, reading sentences evoked higher average HGA than reading words, suggesting active processing of the stimuli rather than comparable visual processing for words and sentences (FIG. 52B).

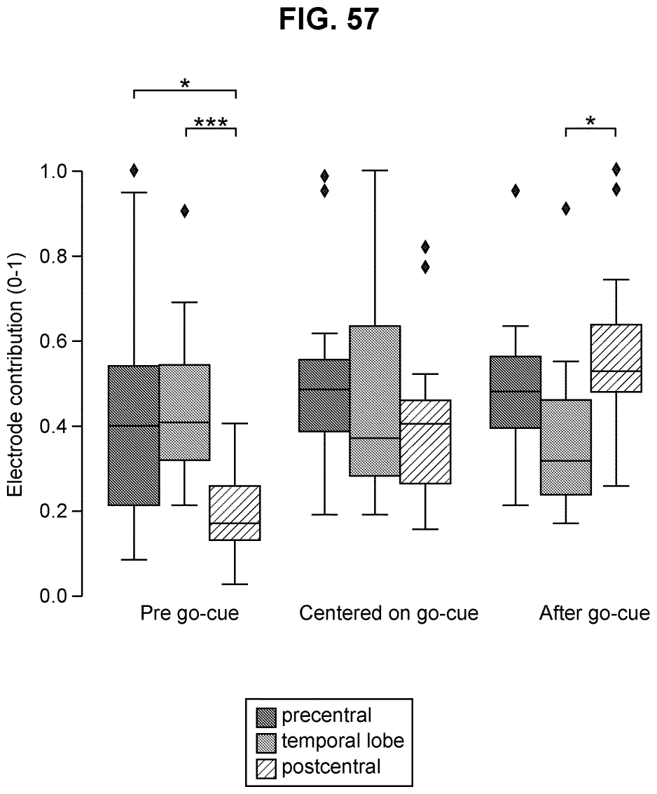

Neural populations with multisensory properties, which are activated across multiple tasks, have been proposed to play a role in higher-level coordination [11,3] of motor actions. Given that shared electrodes in Bravo-3 had multisensory properties, reading activations that were not a result of visual changes and stronger for language (despite reading and listening content not being strongly decodable), HGA above baseline before speech attempts (a requirement of motor planning activity) [49], and a distinct localization from electrodes that encode articulatory movements (FIG. 50B), it was hypothesized that shared electrodes have a specific role in speech-motor planning. To investigate this in the dataset, classifiers were trained on attempted-speech data from three sequential windows around the go-cue in the isolated-target task: one exclusively before the go-cue, one centered on the go-cue, and another exclusively after the go-cue. Indeed, it was found that shared electrodes in the midPrCG and pSTG were most important for decoding attempted speech before the go-cue whereas articulatory encoding electrodes, around the central sulcus, were most important for decoding the after go-cue window (FIG. 52D-52E; FIG. 57). Notably, shared electrodes not only contributed to before go-cue decoding, but their contributions persisted throughout each window, in contrast to articulatory encoding electrodes (FIG. 52E).

FIGS. 52A-52E: A role for shared electrodes in speech-motor planning. FIG. 52A: Shown are example evoked response potentials (ERPs) from two electrodes in Bravo-3 that are task-modulated during reading (as in FIG. 50A). ERPs are aligned to the onset of reading words from the isolated-word set, sentences, and false fonts. FIG. 52B: For each reading-responsive electrode, the average HGA (0-2 s) is scattered between reading words from the isolated-word set and false fonts. FIG. 52C: For each reading-responsive electrode, the average HGA (0-2 s) is scattered between reading words from the isolated-word set and sentences. FIG. 52D: Shown are electrode contributions for attempted-speech models, trained on different windows of time around the go-cuc: strictly before the go-cuc, centered on the go-cuc, and strictly after the go-cue. The black region outline indicates the midPrCG and blue shading the precentral gyrus. FIG. 52E: The average electrode contribution for postcentral, precentral, and temporal-lobe electrodes in each decoding window is quantified. Error bars reflect standard error of the mean.

FIG. 56: Electrodes with comparable HGA for reading words and false fonts localize around the frontal eye fields. For reading-modulated electrodes, the difference in mean HGA during the first 2 seconds of reading real words vs false fonts is shown.

FIG. 57: Anatomical characteristics of electrodes important for decoding attempted speech pre go-cue and after the go-cue. Shown are electrodes contributions by anatomical regions for each decoding model trained on different windows around the go-cue. To be considered in the analysis, an electrode must be within the top 10th percentile of contributing electrodes for at least one of the decoding windows (*P<0.05, ***P<0.001; two-sided Mann-Whitney U tests with Holm-Bonferroni correction).

### 3.4. SUMMARY AND CONCLUSIONS

Across two participants with vocal-tract paralysis, an ECoG-based speech neuroprosthesis was developed that can decode attempted speech with minimal interference from reading, listening, or non-speech mental imagery. With a tailored decoding framework, where models were trained on cortical activity during reading and listening, there were zero false-positive activations of the system during sustained periods of listening, reading, or mental imagery tasks. Furthermore, decoding performance was not disrupted while listening to audio during speech attempts. Offline, it was found that the SMC was activated by reading and listening, but the spectrotemporal properties of these activation patterns were strongly differentiable and supported discriminating between perceptual tasks and attempted speech. Interestingly, in the temporal lobe, attempted speech and listening were both decodable, but decoding performance was driven by largely separate neural populations, supporting the hypothesis that the pSTG may play a crucial role in speech production.

A commonly cited concern, from both potential users and experts in the field, regarding speech neuroprostheses is that they will be activated by and decode internal thoughts or perception that are not meant to be communicated [42,37,9,38,48]. Indeed, one study has shown that listening falsely engaged a speech decoder, resulting in unintended output from the speech decoder [43]. It is important that end-users of the technology retain volitional control over the system to encourage long-term safety, efficacy, and adoption of the technology [24,9]. However, with proper design considerations, perceptual interference with speech decoding can be eliminated or minimized. In one participant, it was also demonstrated that the speech-decoding system was not triggered by non-speech mental imagery. This study builds on principles in value-aligned development to investigate factors cited as important by end-users of the technology. These results may be extended to real-world and clinical scenarios, considering additional sources of interference such as audiovisual stimuli [27] and non-speech orofacial gestures [39,40], such as facial expressions. The results represent a key step towards developing speech-decoding frameworks, ensuring that speech is only decoded when the user desires to speak, encouraging long-term adoption of the technology by those who may benefit.

Though the system was tested using a restricted vocabulary of words, a similar framework may scale to larger vocabulary approaches [29,7,52]. In these cases, a speech-detection model could still be trained on reading, listening, and attempted-speech data to identify volitional speech attempts with low latency and a speech-verification model, trained to evaluate whether full context windows of neural activity are indeed attempted speech, can still be used to gate the onset of large-vocabulary decoding. However, introducing the speech-verification model may add latency, thus necessitating a tradeoff between latency and specificity of the system to attempted speech. Future work, enabled by the preset disclosure, may improve speech-detection frameworks so that a longer-latency speech-verification model is not needed while maintaining low-latency detection. This could be achieved by having a single holistic model to perform speech detection and decoding, such as the case in modern automatic speech recognition algorithms [16].

In terms of the cortical activations that support attempted speech, listening, and reading, while the midPrCG had the most electrodes with shared activations across tasks, it was also a critical region for discriminating perceptual functions from attempted speech. The results suggest that this was due to the differences in the spectrotemporal response patterns for each task, including activations before the onset of speech attempts. Though reading and listening were not strongly decodable from the midPrCG, the midPrCG was important for decoding attempted speech before the go-cue. This aligns with the emerging hypothesis that the midPrCG is critical for speech-motor planning rather than direct execution of articulation [45]. Thus, while not decodable, reading and listening activity in the region may stem from phonological representations that are important for speech-motor planning [50]. The findings are in line with other studies that associate multisensory activity with motor planning [3,11] and studies finding the midPrCG is not tuned to motor execution of a single muscle group [15,18]. Further understanding of the precise nature of representations in the midPrCG, using, e.g., the methods of the present disclosure, may allow speech decoders to leverage complementary information to articulatory representations.

To create reliable speech neuroprostheses that are adopted by people with paralysis, it is important to develop decoding frameworks that are robust to false activations and maintain performance during common perceptual tasks. In this study, a framework was demonstrated that meets these goals and find that electrodes with shared activity for attempted speech and perception may play a specific role in speech-motor planning.

### 3.4. MATERIALS AND METHODS

### 3.4.1. Participants

The first participant (Bravo-1, he/him) was 36 years old at enrollment and had been diagnosed with severe spastic quadriparesis and anarthria by neurologists and a speech-language pathologist due to stroke of the bilateral pons. His injury did not affect cognitive function and he has slight residual function of the vocal tract allowing for audible grunts and moans, though he is unable to produce intelligible speech. To communicate, he relies on an Augmentative and Alternative Communication (AAC) interface that utilizes residual head movements to spell out words.

The second participant (Bravo-3, she/her) was 47 years old at time of enrollment into the study and had been diagnosed with quadriplegia and anarthria by neurologists and a speech-language pathologist. Similarly, this was due to a large stroke of the pons with cognitive function left intact. She can vocalize a small set of monosyllabic sounds, such as ‘ah’ or ‘ooh’, but she is unable to articulate intelligible words. She similarly relies on an AAC interface that leverages residual head movements to facilitate spelling out words. After details concerning the neural implant, experimental protocols, and medical risks were explained, both participants provided full informed consent to partake in the study.

### 3.4.2. Neural Implants

Both participants were implanted with high-density electrocorticography (ECoG) arrays (PMT) and percutaneous pedestal connectors (Blackrock Microsystems). For Bravo-1, the array consisted of 128 electrodes with 4-mm center-to-center spacing implanted on the pial surface, covering a portion of the left frontal, precentral, and postcentral cortex (FIG. 49A). For Bravo-3, the array consisted of 253 electrodes with 3 mm center-to-center spacing implanted on the pial surface covering a portion of the left precentral, postcentral, and temporal cortex (FIG. 49A). The percutaneous connector transmits electrical measurements from the ECoG arrays to an external headstage (Blackrock Microsystems), where signals are digitized and sent to a computer for downstream analysis. Results for this project were collected 1752 days post implant for Bravo-1 and 492 days post implant for Bravo-3.

### 3.4.3. Data Acquisition and Signal Processing

The same data acquisition and signal processing framework was applied as in Example 1. In brief, electrical field potentials from the ECoG contacts were common-average referenced with subsequent extraction of the high-gamma amplitude (HGA; 70-150 Hz) and low-frequency signal (LFS; 1-100 Hz) at each electrode at 200 Hz. A 30-second sliding-window z-score was used to separately normalize each ECoG channel's HGA and LFS. Together, HGA and LFS are referred to as neural features and were used for both training and evaluation of online decoding models. All data collection and online decoding tasks were performed either at or near the participant's residence. Decoding models were trained offline using NVIDIA Tesla V100 GPUS hosted on the lab's server infrastructure. A custom Python real-time package [32] was used to coordinate data collection, task management, and online decoding.

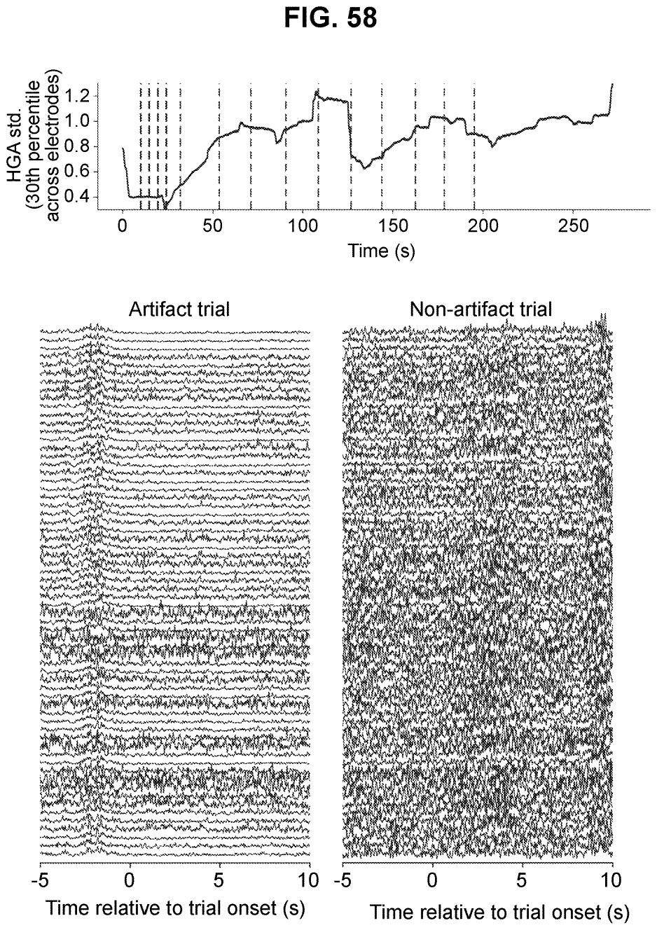

Artifact rejection: In Bravo-1, three trials were excluded due to an artifact affecting the quality of the HGA (FIG. 58). This artifact caused extended periods of decreased variance in the signal. Each trial was examined, both qualitatively and quantitatively, to exclude trials affected by this artifact. Quantitatively, the overall temporal variance was computed across channels by first taking the standard deviation of the HGA for each electrode over the window [−5, 10] relative to every timepoint. Next, as an aggregate measure the 30th percentile of the HGA standard deviation across electrodes at each timepoint was taken. A cut-off of 0.6 for this metric was used to identify trials affected by this artifact. FIG. 58 shows one of the three trials.

FIG. 58: Artifact that affected 3 trials in the online evaluation blocks. Shown is a sample artifact that occurred during three trials of online evaluation with Bravo-1. The standard deviation of the high-gamma amplitude is visualized over time (top). This metric was computed by first taking the HGA standard deviation for each electrode over the window [−5, 10] relative to each timepoint. Next, to quantify whether a proportion of electrodes' had decreased their variance, the 30th percentile of the HGA std across electrodes at each timepoint was taken. Sample trials with and without an artifact (bottom).

### 3.4.4. Task Design

To train and evaluate the speech-decoding system, several different tasks were designed.

Isolated-target task: During the isolated-target task for attempted speech, the text for a target word appeared on the screen along with 4 dots on either side. Each dot sequentially disappeared followed by the text target turning green to indicate the participant should attempt to speak (go-cue). Bravo-1 attempted to speak with some residual vocalization while Bravo-3 silently attempted to speak (attempted miming). During the isolated-target task, a vocabulary of 10 total words (referred to as isolated words) was used for each participant. For Bravo-1 these words were a subset of the words used for a prior bilingual-decoding study [45] (Table 30). In Bravo-3 these words were a subset of the NATO-code words that represent the phonetic alphabet [29]. The same utterance sets were used to probe passive listening and reading. In these cases, one of the isolated words would either be played through the speakers or shown on the screen for the participant to read. For reading and listening, no countdown was used, and a version of the task where 10 sentences, rather than the isolated words, were shown was also collected. In the case of reading, data where false fonts (e.g. “δ [symbol] ) were shown to the participant were also collected [54]. Neural features collected during the isolated-target task were used to train and optimize deep-learning models trained to detect and verify attempted speech as well as classify content of attempted speech, reading, and listening.

TABLE 30
10-word utterance sets for each participant.

| Participant | Utterance | |
|---|---|---|
| Bravo-1 | Pancho | Despierto |
| | Alimento | Vehicle |
| | Enfermero | Speak |
| | Ropa | Funny |
| | Comer | Awake |
| Bravo-3 | X-ray | India |
| | Bravo | Yankee |
| | Kilo | Sierra |
| | Foxtrot | Charlie |
| | Whiskey | Hotel |

Online-decoding task: The goal of the online-decoding task was to test the robustness of the speech-decoding system to perceptual “distractors”. In this task, one of the isolated words appeared on the screen. The participant could then attempt to speak this word whenever desired. After a speech attempt was detected, verified, and classified (FIG. 49B), the screen would be cleared of all text for 4 seconds before the next trial would begin with a new isolated word on the screen. The task was collected in blocks where each of the 10 isolated words was presented once. This task was collected under three conditions (FIG. 49C). In the first “baseline” condition, no perceptual distractor was present. In the second “listening” distractor condition, a podcast was consistently playing throughout the task and the participant took time to listen to the podcast before making a speech attempt. Finally, in the third “reading” distractor condition, a laptop opened to an online article was placed in front of the participant (the experimenter would scroll based on a head cue from the participant). The participant took time to read the article before making each speech attempt.

Speech detection, verification, and classification models were trained offline prior to online-system use with the isolated-target task. Speech detection models were trained to identify attempted speech events from the neural features. Speech verification models were trained to classify whether an isolated-target trial was reading, listening, or attempted speech. Classification models were trained to output the identity of the word based on attempted-speech events.

### 3.4.5. Modeling

The overall speech-decoding system consisted of three deep-learning models. The first was designed to run continuously and detect speech at low latencies. The second was trained to verify a detected speech event as being attempted speech, and not reading or listening. The third was trained to classify detected speech events as one of the isolated words. All model hyperparameters and weights were chosen/trained based on task data from data-collection sessions prior to online evaluations and no recalibration occurred during online evaluations. While no day-of recalibration occurred during online testing, the Bravo-3 online evaluations with the reading distractor occurred over a month after evaluations with the listening distractor. In this case, all models were retrained on data collected during the intermediate month.

Speech detection: The speech detection model was designed and trained similarly to the prior works [30,33,45]. In brief, the speech detection model consisted of 3 unidirectional long short-term memory (LSTM) layers followed by 1 fully connected layer. The model continuously processed incoming neural features at 200 Hz and generated continuous probabilities over 5 classes-silence, preparation, speech, reading, and listening. These probabilities were then processed to generate discrete windows corresponding to detected speech attempts. The processing consisted of smoothing the probabilities with a causal moving average window, thresholding the smoothed probabilities to generate a binary signal, and then a time threshold to determine the onset and offset of attempted speech events. Relevant model parameters are detailed in Table 31.

TABLE 31
Parameters corresponding to the speech detection model.

| Participant | Parameter | Value |
|---|---|---|
| Bravo-1 | Number of LSTM layers | 3 |
| | Nodes per LSTM layer | 128, 128, 32 |
| | Dropout rate | 0.5 |
| | Optimizer | Adam |
| | Learning rate (reduced on plateau) | 0.01 |
| | Smoothing window size | 100 time points |
| | Probability threshold | 0.5 |
| | Time threshold | 100 time points |
| Bravo-3 | Number of LSTM layers | 3 |
| | Nodes per LSTM layer | 128, 96, 32 |
| | Dropout rate | 0.5 |
| | Optimizer | Adam |
| | Learning rate (reduced on plateau) | 0.001 |
| | Smoothing window size | 100 time points |
| | Probability threshold | 0.5 |
| | Time threshold | 100 time points |

For training, labels corresponding to each of the 5 target classes were manually created following the same general principles, with some adjustments per participant. For the isolated words task, time points between the presentation of the word on the screen and the go-cue were labeled as “speech preparation”. Time points from the go-cue to some offset from the go-cue (for Bravo-1, to 2.75 seconds after go-cue, for Bravo-3, to 85% of the trial duration) were labeled as “speech”. Time points from this go-cue offset to the end of the trial (before the screen cleared for an inter-trial interval) were not used during training as it is ambiguous when each participant had finished the speech attempt and returned to rest. These offsets were chosen based on qualitative observations of each participant's behavior. All other time points were labeled as “silence”. For the listening task, all time points during the stimulus presentation were labeled as “listen” and the remaining time points as “silence”. For the reading task, all time points when the target word was on the screen were labeled as “read” and the remaining time points as “silence”. In addition to the listening and reading versions of the isolated-words tasks, each participant also listened and read a set of 10 phrases. These were labeled in the same way. Additionally, 1 minute-long blocks were collected with each participant where they were instructed to rest. All time points in these blocks were labeled as “silence” and used during training.

For Bravo-1, model performance qualitatively appeared to be better when not including reading in the training set, and so no reading data was included. Additionally, several test runs of the online decoding task (no distractor) infrastructure were piloted with Bravo-1. As these blocks had no explicit go-cues, the acoustic onset and offset of his speech attempts were manually annotated, labeled as speech and all other time points as silence, and used during training. Online evaluation blocks were collected on a single day, with the exception of the long memory-reflection block which was collected 48 days later. The same online model was used in both cases.

For Bravo-3, various phrase-level data was being collected alongside the aforementioned datasets. For convenience, more recent phrase-level data was labeled according to the methods stated above and used as the primary source of attempted speech examples. Online evaluation blocks were collected on two separate days, 43 days apart. For the first day, the online speech-detection model was trained with the phrase-level speech-attempt data, 10-phrase and 10-word listening data, rest data, and 10-word reading data (10-phrase reading data was accidentally excluded). For the second day, the same dataset plus 10-word speech attempt data and 10-phrase reading data was also used to retrain a new model.

In all cases, models used for online evaluation were only trained on data prior to that day.

Speech-detection ablations: In order to assess whether including examples of reading and listening helped reduce any potential false positives, versions of the speech-detection model that did not use reading or listening data were also trained and evaluated offline on the evaluation blocks. For Bravo-1, a single model was retrained, which used a subset of the data used to train the online model (e.g. just removing listening data). For Bravo-3, two models were retrained as two models were used for online evaluation. The first model was trained using a subset of the data that the online model used on the first day of online evaluations-excluding reading and listening data, however also including prior isolated words data. The second model was similarly trained using a subset of the data used to train the online model used on the second day of evaluations. These models were used to offline evaluate the blocks from the first and second day, respectively. No model parameters were changed for these ablations.

Latency of speech-detection model: In order to generate discrete detected events that correspond to speech attempts, continuously predicted probabilities (at 200 Hz) go through aforementioned post-processing. While smoothing and probability thresholding are causal or do not impose latency, time threshold does impose a latency as it must wait for the smoothed predicted speech probability to be above a certain threshold (the probability threshold parameter) for at least a certain number of time points (the time threshold parameter). Similarly, the probability must return to below this threshold for at least a certain number of time points to determine the offset of the event. In this study, those parameters were set to 100 time points, or 0.5s. If detected events were perfectly aligned to the onset and offset of speech attempts, there is still a 0.5s latency before the onset and offsets are each recognized, yielding a consistent 0.5s latency to detecting an onset or offset in addition to the latency of those from the onset or offset of the true speech attempt.

For Bravo-1, the onset and offset of his speech attempts could be measured by annotating his vocalizations. It was found that there was a median 0.14s onset latency and a median 0.89s offset latency. For Bravo-3, since she silently attempted to speak, the latency could not similarly be computed based on vocalization time.

Though in this study, discrete events were detected and classified, the speech detection model is amenable to lower latency frameworks cither by reducing the time threshold parameter or using the continuously predicted speech probabilities at 200 Hz.

Speech verification: Speech verification models were trained with the objective to classify a detected speech event as being either attempted speech, listening, or reading. To train such a model, attempted speech, listening, and reading data from the isolated-target task was used. For reading and listening, neural features were included from both isolated words and sentences (false fonts were not included) aligned to the onset. For attempted speech data, rather than aligning to the go-cue, the detected onset of each trial was aligned to (see above, ‘Speech detection’). For Bravo-1, a few practice online-decoding blocks were collected a week before testing and these trials were included in training. In all cases, the speech-verification model was trained on a window of [−0.5,3.5] s and [−1,3] s around the trial onset for Bravo-1 and Bravo-3 respectively. Similar to prior work [23,30], the model itself consumed neural features at a rate of 33.3 Hz (downsampled by a factor of 6 from 200 Hz) and consisted of a one-dimensional temporal convolutional layer followed by 2 gated recurrent neural network (GRU) layers with a final fully-connected layer that produces probabilities over 3 classes: attempted speech, reading, and listening. During model training, stochastic gradient descent and the Adam optimizer [22] were used to find a set of parameters that minimized the cross entropy loss between target and predicted outputs. During online testing, any event with an attempted-speech probability of over 0.65 was verified to be associated with attempted-speech. This was chosen based on probability distribution from the isolated-target task. A table containing the precise hyperparameters used is provided in Table 32.

TABLE 32
Parameters corresponding to the speech-verification model.

| Participant | Parameter | Value |
|---|---|---|
| Bravo-1 | Number of GRU layers | 2 |
| | Nodes per GRU layer | 220 |
| | Dropout rate | 0.7 |
| | CNN Kernel size and stride | 8 |
| | Time window | [−0.5, 3.5] |
| | weight decay | 0.0001 |
| | Optimizer | Adam |
| | Learning rate (reduced on plateau) | 0.0005 |
| Bravo-3 | Number of GRU layers | 2 |
| | Nodes per GRU layer | 220 |
| | Dropout rate | 0.7 |
| | CNN Kernel size and stride | 8 |
| | Time window | [−1, 3] |
| | weight decay | 0.0001 |
| | Optimizer | Adam |
| | Learning rate (reduced on plateau) | 0.0005 |

Classification: Classification models were trained on the attempted-speech data from the isolated-targets task and a few practice online-decoding blocks (as in ‘Speech verification’) for Bravo-1. Neural features were aligned to detected onsets for each trial and a window of [−0.5,3] s and [−1,3] s was used for Bravo-1 and Bravo-3 respectively. The classification architecture and process was the same as Example 1. Neural features were first downsampled by a factor of 6 before being passed through a one-dimensional temporal convolutional layer and a series of GRU layers (2 for Bravo-3, 3 for Bravo-1). A fully-connected layer of output dimension 10 was used to map the final latent state from the GRU layers onto a distribution over the isolated words. The softmax function was used to scale this output to a probability distribution. Similar to above, model training was guided by stochastic gradient descent and the Adam optimizer to find a set of parameters that minimized cross entropy loss between target and predicted outputs. For both online classification and speech-verification, 10 randomly initialized models were fit on the training data to improve prediction accuracy via ensembling. A table containing the precise hyperparameters used is provided in Table 33.

TABLE 33
Parameters corresponding to the speech-classification model.

| Participant | Parameter | Value |
|---|---|---|
| Bravo-1 | Number of GRU layers | 3 |
| | Nodes per GRU layer | 260 |
| | Dropout rate | 0.8 |
| | CNN Kernel size and stride | 4 |
| | Time window | [−0.5, 3.5] |
| | weight decay | 0.0001 |
| | Optimizer | Adam |
| | Learning rate (reduced on plateau) | 0.0001 |
| Bravo-3 | Number of GRU layers | 2 |
| | Nodes per GRU layer | 274 |
| | Dropout rate | 0.54 |
| | CNN Kernel size and stride | 4 |
| | Time window | [−1, 3] |
| | weight decay | 0.0001 |
| | Optimizer | Adam |
| | Learning rate (reduced on plateau) | 0.0005 |

### 3.4.6. Evaluation

To evaluate the speech-decoding system in the presence of perceptual interference, 9 total blocks of the online-decoding task were collected: 3 baseline, 3 with reading, and 3 with listening. This gave a total of 30 trials for each condition with 3 repeats of each isolated word. In Bravo-3 a fourth baseline block was collected, giving 40 total trials. In Bravo-1, the three trials affected by the artifact were excluded (FIG. 58). Additionally, to further probe the specificity of the system to attempted speech over longer time scales, online-decoding blocks where the participant listened to a podcast, read articles, or performed a mental imagery task for several minutes were collected (see FIG. 49L). During these blocks, however, the participant was instructed to not make any speech attempts.

True positive and false positive calculations: The true positive rate (TPR) is defined as the proportion of true speech attempts that were correctly detected by the decoding system while the false positive rate (FPR) is defined as the proportion of detected events that do not correspond to a true speech attempt. To identify true and false positive detected events, all true speech attempts were first manually annotated. For Bravo-1, this was done based on the recorded audio while for Bravo-3, this was done from face camera recordings (as she silently attempts to speak while Bravo-1 audibly attempts to speak). If a detected event is within 0.5 seconds of a true speech attempt, it is assigned to that speech attempt and would be considered a true positive. If one or more detected events meet this criteria for a single speech attempt, they are all assigned to that speech attempt. This does not affect the calculation of TPR, as that speech attempt is still counted as a true positive only once. For the calculation of FPR, only 1 of the detections associated with that speech attempt is counted. This is because including both detections would increase the denominator and could make the FPR appear better than it may truly be.

Offline characterizations of the speech-verification model: To further characterize the speech-verification model, the online evaluation blocks for Bravo-1 and Bravo-3. As a first characterization, the speech-probability threshold was varied between 0 and 1 and the corresponding false positive and false negative rate was computed (as a proportion of total evaluation trials) using the online speech-verification models. As a second characterization, the context length of time windows around detected onsets that were passed to the speech-verification model were restricted. The accuracy of the model in accepting or rejecting the online detected events were then computed, using the speech-probability thresholds found optimal offline (FIG. 49G). This allowed probing of speech-verification performance as a function of context length following a detected event.

Pseudo-blocking trials from online results: Since there were only up to 30 (and for one condition 40 for Bravo-3) trials for the online results for each condition, a pseudo-blocking method was used in order to visualize distributions of TPR, FPR, and 10-word classification accuracy. 6 pseudo-blocks were generated per condition (therefore an average of 5 trials per pseudo-block), and then TPR, FPR, and classification accuracy was calculated for each pseudo-block. These 6 points were used for visualization FIG. 49. Though pseudo-blocks were created for visualization purposes, they were not used for statistical analysis (see Statistics section).

Offline cross validation of classification models: To evaluate the performance of speech-verification and classification models across different regions, a cross validation approach was used over all the isolated-target data. In brief, the above-described training procedure was repeated, using neural features only from electrodes in a given region. The models were evaluated using 10-fold cross validation (CV). In each of the 10-folds, 90% of the data is used for training and 10% for evaluation. Among the 90% of data used for training, an additional 10% of that is reserved as a validation set, used to define when to stop neural network training. Within each of the 10 folds, 5 randomly initialized models were fit to ensemble predictions over the held out evaluation fold.

Neural signal analyses: To measure activation in response to attempted-speech, reading, and listening at each electrode, the data from the isolated-target task was used. Instead of a running 30s z-score, used for online decoding, each trial was z-scored to the silent, inter-trial period before stimulus presentation. For each task, responsiveness was then measured at every electrode based on Wilcoxon rank-sum tests comparing the average HGA during the pre-stimulus window to the average HGA after onset of the function ([0,1] s for attempted speech and reading and [0.5,1] s for listening). For each function, p-values across the 253 electrodes were corrected for multiple comparisons with the Holm-bonferroni method and a cutoff of 0.05 was used for statistical significance. For visualization of task-modulated activation, the Z-statistic at each electrode was visualized with negative statistics (indicating higher baseline activity) set to 0. To compare electrodes with multi-function activations to those that encoded articulatory movements, estimates of supralaryngeal encoding for Bravo-3 were used (methodology for how these values were computed can be seen in Example 1).

For low-frequency band analyses, power spectral density was estimated on the isolated-target data during attempted speech, reading, and listening using a multi-taper method in the theta (5-12 Hz) and beta (20-30 Hz) ranges. For every trial the mean power in these ranges was computed for the 0-2 second window relative to task go-cue or onset. Trials were then pseudo-blocked by taking the mean over consecutive, non-overlapping groups of 60 trials. Distributions across each task were then z-scored for each band.

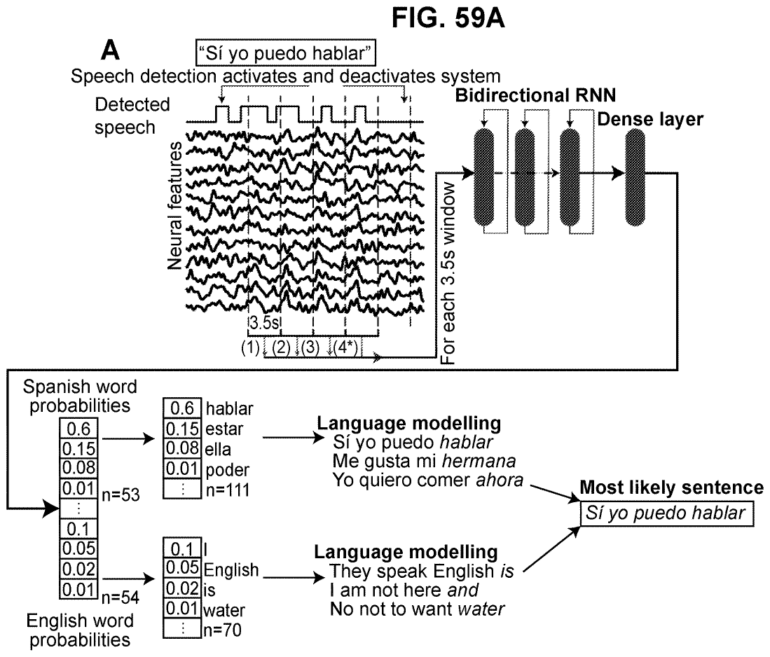

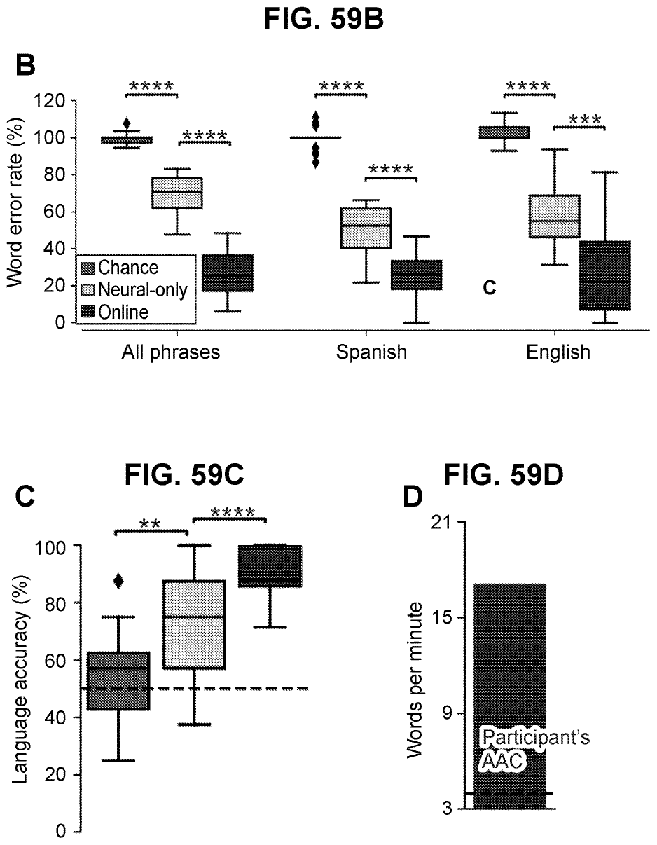

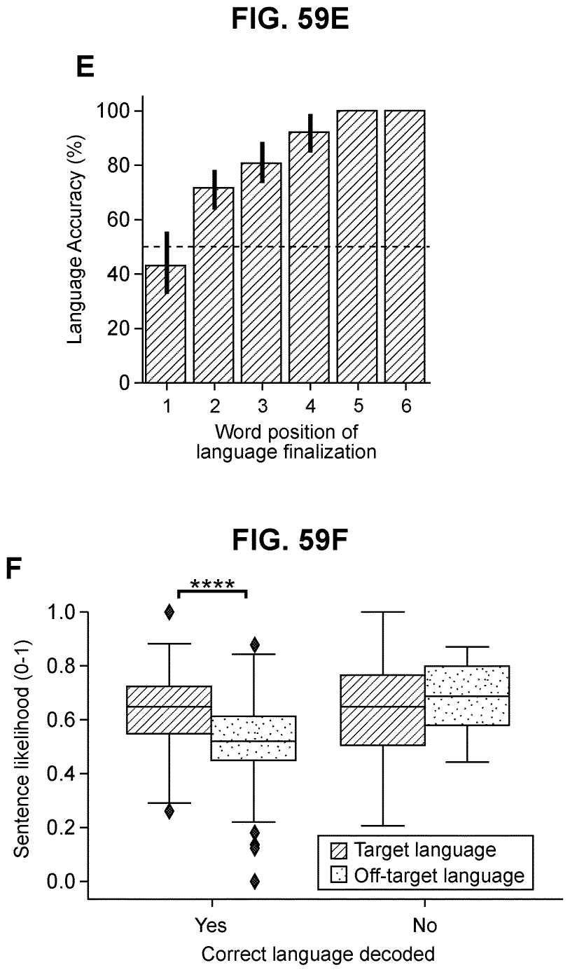

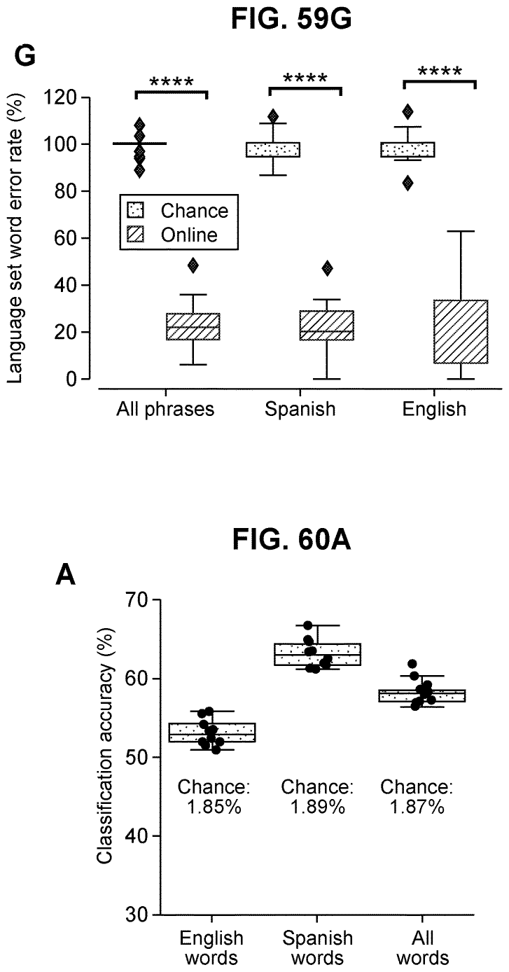

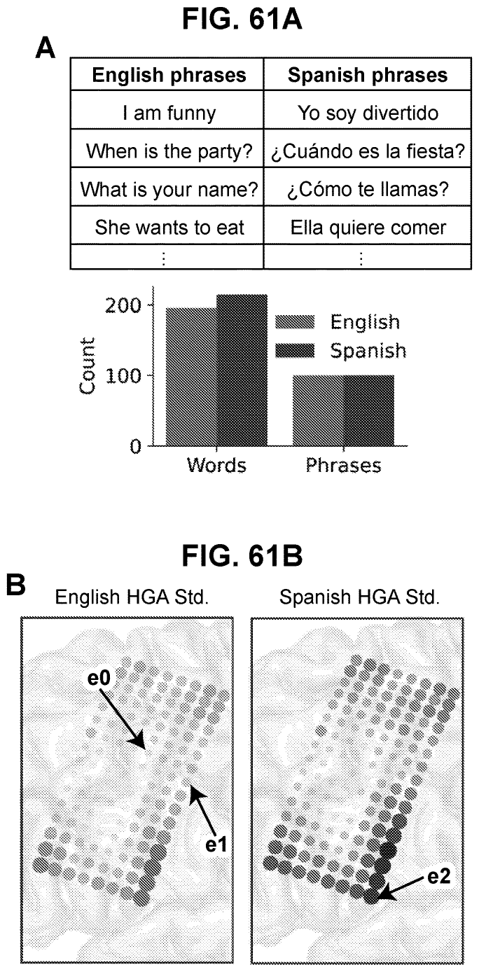

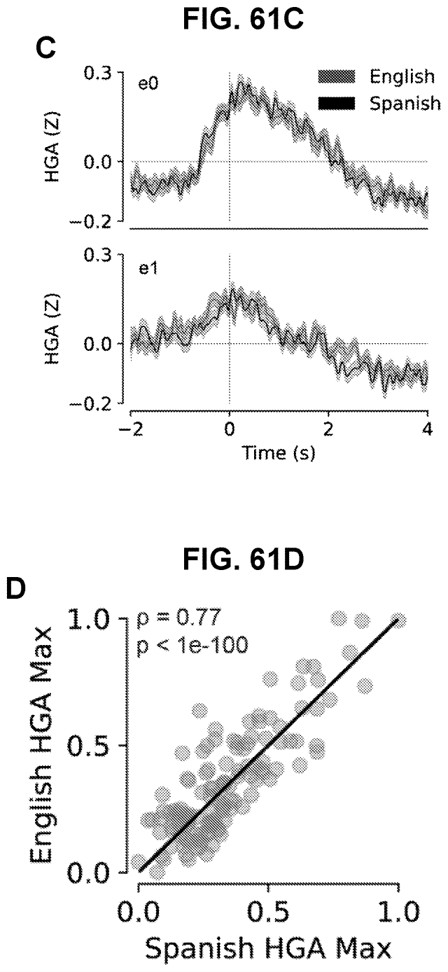

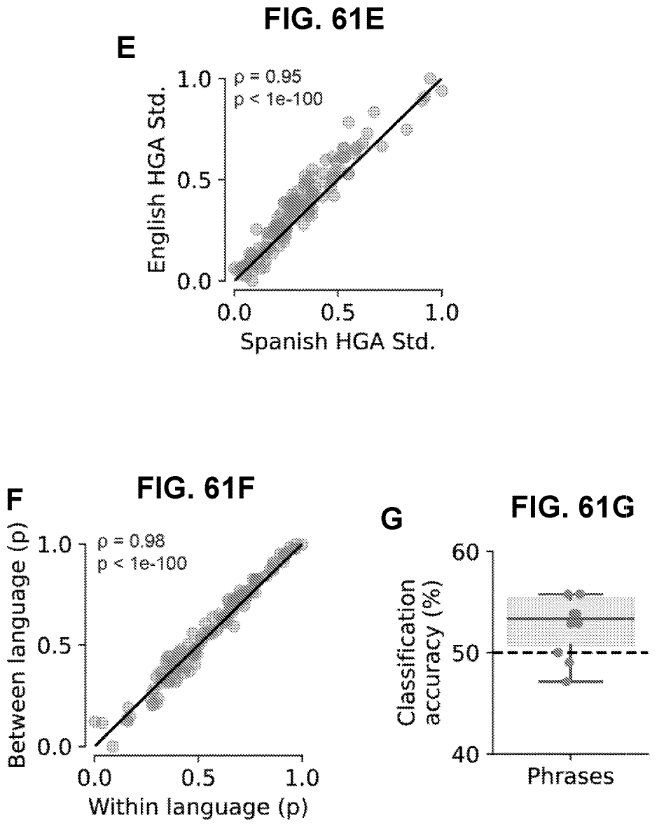

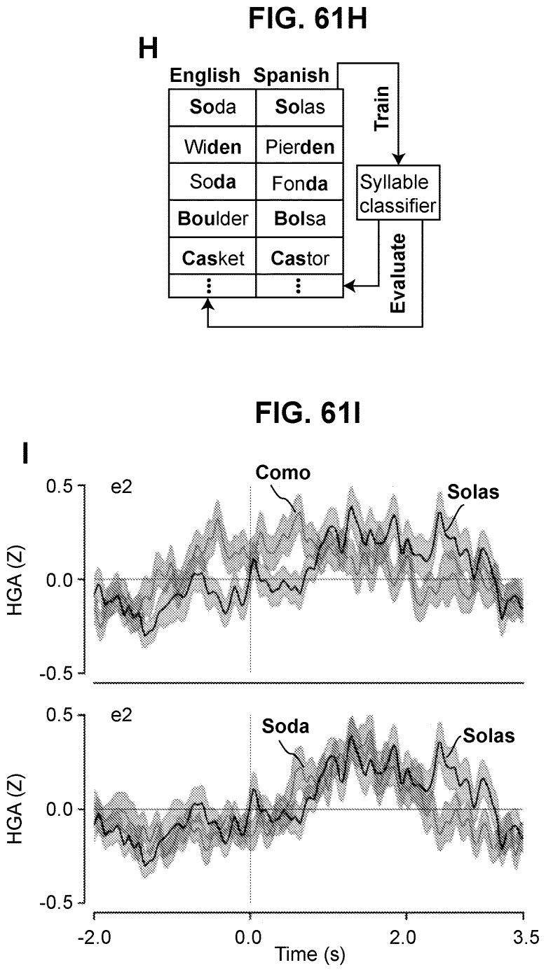

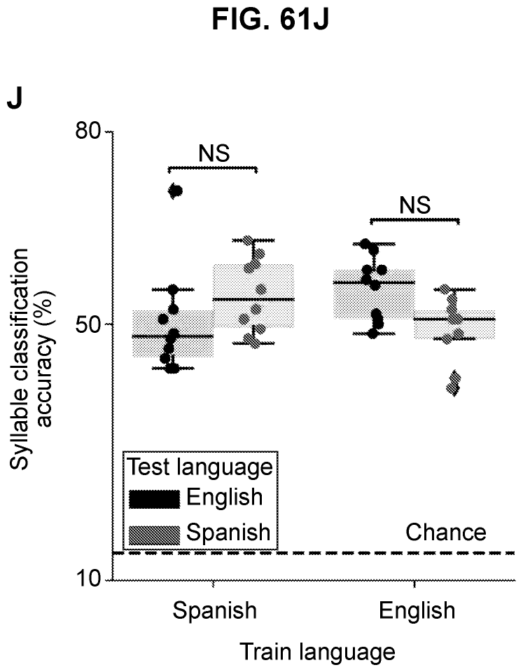

Electrode contributions: For speech detection, speech verification, and 10-word classification models, electrodes contributions were calculated by first taking the derivative of the classifier's loss function with respect to the input features over time [46,29,30]. This captures the change in the model's output given changes in the neural features at each electrode. For each electrode, the L2-norm was computed over the time-dimension of the derivative. This resulted in a single magnitude per electrode per time course. For speech verification and 10-word classification models, these time courses were for each trial. For speech detection, these time courses were over each block. These values were then averaged across time courses yielding a single contribution score per electrode. For speech detection, electrode contributions corresponding to the HGA features are plotted in FIG. 59. Given that LFS substantially improved classification performance in prior work [29], for classification and speech verification, this process was performed separately for HGA and LFS features and for each electrode the contribution score is defined by the sum of the two. To quantify mean contribution of shared and articulatory encoding electrodes in FIG. 61, only electrodes that had contributions in the top 10th percentile for at least one time window (pre go-cue, centered on go-cue, after go-cue) were considered. The electrodes contributions for the classification models in FIG. 59 are plotted on a log scale to better visualize the overall distribution of contributions.

### 3.4.7. Statistics

All statistical evaluations were performed using nonparametric tests and noted alongside significance statements in figure captions and the results section. For the online results, because there were much fewer trials, the Fisher exact test was used to compare between conditions for each participant for the TPR, FPR, and classification accuracies. For offline results on the isolated target data, where there are many more trials, Wilcoxon Rank-sum tests are used. Associated p-values for Spearman rank or Pearson correlation coefficients were computed via permutation testing (1000 iterations). For any multiple comparisons, p-values are corrected (within participants) using Holm-Bonferroni correction.

### 3.5. RECORDED VIDEOS

Video 1: Speech decoding with a reading distractor in Bravo-3. An online demonstration of speech-decoding with a reading distractor in Bravo-3. The participant attempts to speak the target word on the screen at her own pace. After choosing to stop reading an article on the computer screen, the participant makes a speech attempt. This speech attempt is detected from neural activity and the corresponding neural activity during the speech attempt is passed to a speech-verification model. If the speech-verification model assigns a high probability to the event being attempted speech and not reading or listening, the neural activity is passed to a classifier that outputs the most likely word in the vocabulary. The system retains high-specificity to attempted speech without false positive activations during the reading phase of the task.

Video 2: Speech decoding with a listening distractor in Bravo-1. The same decoding framework as described in Video 1; however, now the participant, Bravo-1, listens to a podcast and decides when to attempt to speak. The system again retains high-specificity to attempted speech without false positive activations during the listening phase of the task.

Video 3: System specificity for attempted speech during listening in Bravo-1. In this video, Bravo-1 listens to a podcast with the speech-decoding system, described in Videos 1 and 2, active. The speech-decoding system is not activated over several minutes of listening, maintaining high-specificity for attempted speech.

### 3.6. REFERENCES

The numbering related to the following references apply with respect to the experimental results presented in Example 3:
[1]_Akbari, Hassan, Bahar Khalighinejad, Jose L. Herrero, Ashesh D. Mehta, and Nima Mesgarani. 2019. “Towards Reconstructing Intelligible Speech from the Human Auditory Cortex.” Scientific Reports 9 (1): 874. https://doi.org/10.1038/s41598-018-37359-z.
[2]_Alford, John. 2014. “The Multiple Facets of Co-Production: Building on the Work of Elinor Ostrom.” Public Management Review 16 (3): 299-316. https://doi.org/10.1080/14719037.2013.806578.
[3]_Andersen, Richard A., Lawrence H. Snyder, David C. Bradley, and Jing Xing. 1997. “MULTIMODAL REPRESENTATION OF SPACE IN THE POSTERIOR PARIETAL CORTEX AND ITS USE IN PLANNING MOVEMENTS.” Annual Review of Neuroscience 20 (1): 303-30. https://doi.org/10.1146/annurev.neuro.20.1.303.
[4]_Angrick, Miguel, Shiyu Luo, Qinwan Rabbani, Daniel N. Candrea, Samyak Shah, Griffin W. Milsap, William S. Anderson, et al. 2023. “Online Speech Synthesis Using a Chronically Implanted Brain-Computer Interface in an Individual with ALS.” medRxiv. https://doi.org/10.1101/2023.06.30.23291352.
[5]_Binder, Jeffrey R. 2015. “The Wernicke Area.” Neurology 85 (24): 2170-75. https://doi.org/10.1212/WNL.0000000000002219.
[6]_Binder, Jeffrey R. 2017. “Current Controversies on Wernicke's Area and Its Role in Language.” Current Neurology and Neuroscience Reports 17 (8): 58. https://doi.org/10.1007/s11910-017-0764-8.
[7]_Card, Nicholas S., Maitreyee Wairagkar, Carrina Iacobacci, Xianda Hou, Tyler Singer-Clark, Francis R. Willett, Erin M. Kunz, et al. 2023. “An Accurate and Rapidly Calibrating Speech Neuroprosthesis.” Preprint. Neurology. https://doi.org/10.1101/2023.12.26.23300110.
[8]_Cassim, François, Christelle Monaca, William Szurhaj, Jean-Louis Bourriez, Luc Defebvre, Philippe Derambure, and Jean-Daniel Guieu. 2001. “Does Post-Movement Beta Synchronization Reflect an Idling Motor Cortex?” NeuroReport 12 (17): 3859.
[9]_Chandler, Jennifer A., Kiah I. Van der Loos, Susan Boehnke, Jonas S. Beaudry, Daniel Z. Buchman, and Judy Illes. 2022. “Brain Computer Interfaces and Communication Disabilities: Ethical, Legal, and Social Aspects of Decoding Speech From the Brain.” Frontiers in Human Neuroscience 16 (April). https://doi.org/10.3389/fnhum.2022.841035.
[10]_Cheung, Connie, Liberty S Hamilton, Keith Johnson, and Edward F Chang. 2016. “The Auditory Representation of Speech Sounds in Human Motor Cortex.” Edited by Barbara G Shinn-Cunningham. eLife 5 (March): e12577. https://doi.org/10.7554/eLife.12577.
[11]_Cooke, Dylan F., and Michael S. A. Graziano. 2004. “Sensorimotor Integration in the Precentral Gyrus: Polysensory Neurons and Defensive Movements.” Journal of Neurophysiology 91 (4): 1648-60. https://doi.org/10.1152/jn.00955.2003.
[12]_Das, Joe M., Kingsley Anosike, and Ria Monica D. Asuncion. 2021. Locked-in Syndrome. StatPearls [Internet]. StatPearls Publishing. https://www.ncbi.nlm.nih.gov/books/NBK559026/.
[13]_Duffy, Joseph R. 2019. Motor Speech Disorders: Substrates, Differential Diagnosis, and Management. Elsevier Health Sciences.
[14]_Glasser, Matthew F., Timothy S. Coalson, Emma C. Robinson, Carl D. Hacker, John Harwell, Essa Yacoub, Kamil Ugurbil, et al. 2016. “A Multi-Modal Parcellation of Human Cerebral Cortex.” Nature 536 (7615): 171-78. https://doi.org/10.1038/nature18933.
[15]_Gordon, Evan M., Roselyne J. Chauvin, Andrew N. Van, Aishwarya Rajesh, Ashley Nielsen, Dillan J. Newbold, Charles J. Lynch, et al. 2023. “A Somato-Cognitive Action Network Alternates with Effector Regions in Motor Cortex.” Nature, April 1-9. https://doi.org/10.1038/s41586-023-05964-2.
[16]_He, Yanzhang, Tara N. Sainath, Rohit Prabhavalkar, Ian McGraw, Raziel Alvarez, Ding Zhao, David Rybach, et al. 2019. “Streaming End-to-End Speech Recognition for Mobile Devices.” In ICASSP 2019-2019 IEEE International Conference on Acoustics, Speech and Signal Processing (ICASSP), 6381-85. https://doi.org/10.1109/ICASSP.2019.8682336.
[17]_Hickok, Gregory, and David Poeppel. 2007. “The Cortical Organization of Speech Processing.” Nature Reviews Neuroscience 8 (5): 393-402. https://doi.org/10.1038/nrn2113.
[18]_Jensen, Michael A., Harvey Huang, Gabriela Ojeda Valencia, Bryan T. Klassen, Max A. van den Boom, Timothy J. Kaufmann, Gerwin Schalk, et al. 2023. “A Motor Association Area in the Depths of the Central Sulcus.” Nature Neuroscience 26 (7): 1165-69. https://doi.org/10.1038/s41593-023-01346-z.
[19]_Kaestner, Erik, Thomas Thesen, Orrin Devinsky, Werner Doyle, Chad Carlson, and Eric Halgren. 2021. “An Intracranial Electrophysiology Study of Visual Language Encoding: The Contribution of the Precentral Gyrus to Silent Reading.” Journal of Cognitive Neuroscience 33 (11): 2197-2214. https://doi.org/10.1162/jocn_a_01764.
[20]__Kaestner, Erik, Xiaojing Wu, Daniel Friedman, Patricia Dugan, Orrin Devinsky, Chad Carlson, Werner Doyle, Thomas Thesen, and Eric Halgren. 2022. “The Precentral Gyrus Contributions to the Early Time-Course of Grapheme-to-Phoneme Conversion.” Neurobiology of Language 3 (1): 18-45. https://doi.org/10.1162/nol_a_00047.
[21]_Keitel, Anne, Joachim Gross, and Christoph Kayser. 2018. “Perceptually Relevant Speech Tracking in Auditory and Motor Cortex Reflects Distinct Linguistic Features.” Edited by Jennifer Bizley. PLOS Biology 16 (3): e2004473. https://doi.org/10.1371/journal.pbio.2004473.
[22]_Kingma, Diederik P., and Jimmy Ba. 2017. “Adam: A Method for Stochastic Optimization.” arXiv: 1412.6980 [Cs], January. http://arxiv.org/abs/1412.6980.
[23]_Krauss, Robert M., and Peter D. Bricker. 1967. “Effects of Transmission Delay and Access Delay on the Efficiency of Verbal Communication.” The Journal of the Acoustical Society of America 41 (2): 286-92. https://doi.org/10.1121/1.1910338.
[24]_Kübler, Andrea, Elisa M. Holz, Angela Riccio, Claudia Zickler, Tobias Kaufmann, Sonja C. Kleih, Pit Staiger-Sälzer, Lorenzo Desideri, Evert-Jan Hoogerwerf, and Donatella Mattia. 2014. “The User-Centered Design as Novel Perspective for Evaluating the Usability of BCI-Controlled Applications.” PLOS ONE 9 (12): e112392. https://doi.org/10.1371/journal.pone.0112392.
[25]_Liberman, A. M., F. S. Cooper, D. P. Shankweiler, and M. Studdert-Kennedy. 1967. “Perception of the Speech Code.” Psychological Review 74 (6): 431-61. https://doi.org/10.1037/h0020279.
[26]_Luo, Shiyu, Miguel Angrick, Christopher Coogan, Daniel N. Candrea, Kimberley Wyse-Sookoo, Samyak Shah, Qinwan Rabbani, et al. 2023. “Stable Decoding from a Speech BCI Enables Control for an Individual with ALS without Recalibration for 3 Months.” Advanced Science (Weinheim, Baden-Wurttemberg, Germany), October, e2304853. https://doi.org/10.1002/advs.202304853.
[27]_Meer, Johan N. van der, Michael Breakspear, Luke J. Chang, Saurabh Sonkusare, and Luca Cocchi. 2020. “Movic Vicwing Elicits Rich and Reliable Brain State Dynamics.” Nature Communications 11 (1): 5004. https://doi.org/10.1038/s41467-020-18717-w.
[28]_Mesgarani, Nima, Connie Cheung, Keith Johnson, and Edward F. Chang. 2014. “Phonetic Feature Encoding in Human Superior Temporal Gyrus.” Science 343 (6174): 1006-10. https://doi.org/10.1126/science.1245994.
[29]_Example 1: A High-Performance Neuroprosthesis for Speech Decoding and Avatar Control (Above).
[30]_Metzger, Sean L., Jessie R. Liu, David A. Moses, Maximilian E. Dougherty, Margaret P. Seaton, Kaylo T. Littlejohn, Josh Chartier, et al. 2022. “Generalizable Spelling Using a Speech Neuroprosthesis in an Individual with Severe Limb and Vocal Paralysis.” Nature Communications 13 (1): 6510. https://doi.org/10.1038/s41467-022-33611-3.
[31]_Meyer, Lars. 2018. “The Neural Oscillations of Speech Processing and Language Comprehension: State of the Art and Emerging Mechanisms.” European Journal of Neuroscience 48 (7): 2609-21. https://doi.org/10.1111/ejn.13748.
[32]_Moses, David A., Matthew K. Leonard, and Edward F. Chang. 2018. “Real-Time Classification of Auditory Sentences Using Evoked Cortical Activity in Humans.” Journal of Neural Engineering 15 (3). https://doi.org/10.1088/1741-2552/aaab6f.
[33]_Moses, David A., Sean L. Metzger, Jessie R. Liu, Gopala K. Anumanchipalli, Joseph G. Makin, Pengfei F. Sun, Josh Chartier, et al. 2021. “Neuroprosthesis for Decoding Speech in a Paralyzed Person with Anarthria.” New England Journal of Medicine 385 (3): 217-27. https://doi.org/10.1056/NEJMoa2027540.
[34]_Peters, B, G Bieker, SM Heckman, JE Huggins, C Wolf, D Zeitlin, and M Fried-Oken. 2015. “Brain-Computer Interface Users Speak Up: The Virtual Users' Forum at the 2013 International Brain-Computer Interface Meeting.” Archives of Physical Medicine and Rehabilitation 96 (3 0): S33-37. https://doi.org/10.1016/j.apmr.2014.03.037.
[35]_Pulvermüller, Friedemann, and Luciano Fadiga. 2010. “Active Perception: Sensorimotor Circuits as a Cortical Basis for Language.” Nature Reviews Neuroscience 11 (5): 351-60. https://doi.org/10.1038/nrn2811.
[36]_Pulvermüller, Friedemann, Martina Huss, Ferath Kherif, Fermin Moscoso del Prado Martin, Olaf Hauk, and Yury Shtyrov. 2006. “Motor Cortex Maps Articulatory Features of Speech Sounds.” Proceedings of the National Academy of Sciences 103 (20): 7865-70. https://doi.org/10.1073/pnas.0509989103.
[37]_Rainey, Stephen, Stéphanie Martin, Andy Christen, Pierre Megevand, and Eric Fourneret. 2020. “Brain Recording, Mind-Reading, and Neurotechnology: Ethical Issues from Consumer Devices to Brain-Based Speech Decoding.” Science and Engineering Ethics 26 (4): 2295-2311. https://doi.org/10.1007/s11948-020-00218-0.
[38]_Rainey, Stephen, Hannah Maslen, Pierre Megevand, Luc H. Arnal, Eric Fourneret, and Blaise Yvert. 2019. “Neuroprosthetic Speech: The Ethical Significance of Accuracy, Control and Pragmatics.” Cambridge Quarterly of Healthcare Ethics 28 (4): 657-70. https://doi.org/10.1017/S0963180119000604.
[39]_Salari, E., Z. V. Freudenburg, M. P. Branco, E. J. Aarnoutse, M. J. Vansteensel, and N. F. Ramsey. 2019. “Classification of Articulator Movements and Movement Direction from Sensorimotor Cortex Activity.” Scientific Reports 9 (1): 14165. https://doi.org/10.1038/s41598-019-50834-5.
[40]_Salari, Efraim, Zachary V. Freudenburg, Mariska J. Vansteensel, and Nick F. Ramsey. 2020. “Classification of Facial Expressions for Intended Display of Emotions Using Brain-Computer Interfaces.” Annals of Neurology 88 (3): 631-36. https://doi.org/10.1002/ana.25821.
[41]_Sanes, J. N., and J. P. Donoghue. 1993. “Oscillations in Local Field Potentials of the Primate Motor Cortex during Voluntary Movement.” Proceedings of the National Academy of Sciences of the United States of America 90 (10): 4470-74. https://doi.org/10.1073/pnas.90.10.4470.
[42]_Sankaran, Narayan, David Moses, Winston Chiong, and Edward F. Chang. 2023. “Recommendations for Promoting User Agency in the Design of Speech Neuroprostheses.” Frontiers in Human Neuroscience 17 (October): 1298129. https://doi.org/10.3389/fnhum.2023.1298129.
[43]_Schippers, A., M. J. Vansteensel, Z. V. Freudenburg, and N. F. Ramsey. 2024. “Don't Put Words in My Mouth: Speech Perception Can Generate False Positive Activation of a Speech BCI.” medRxiv. https://doi.org/10.1101/2024.01.21.23300437.
[44]_Silva, Alexander B., Jessie R. Liu, Sean L. Metzger, Ilina Bhaya-Grossman, Maximilian E. Dougherty, Margaret P. Seaton, Kaylo T. Littlejohn, et al. 2024. “A Spanish-English Speech Neuroprosthesis Driven by Multilingual Cortical Articulatory Representations.” Nature Biomedical Engineering (To Appear).
[45]_Silva, Alexander B., Jessie R. Liu, Lingyun Zhao, Deborah F. Levy, Terri L. Scott, and Edward F. Chang. 2022. “A Neurosurgical Functional Dissection of the Middle Precentral Gyrus during Speech Production.” Journal of Neuroscience 42 (45): 8416-26. https://doi.org/10.1523/JNEUROSCI.1614-22.2022.
[46]_Simonyan, Karen, Andrea Vedaldi, and Andrew Zisserman. 2014. “Deep Inside Convolutional Networks: Visualising Image Classification Models and Saliency Maps.” arXiv:1312.6034 [Cs], April. http://arxiv.org/abs/1312.6034.
[47]_Slepian, D. 1978. “Prolate Spheroidal Wave Functions, Fourier Analysis, and Uncertainty-V: The Discrete Case.” Bell System Technical Journal 57 (5): 1371-1430. https://doi.org/10.1002/j.1538-7305.1978.tb02104.x.
[48]_Sun, Xiao-yu, and Bin Ye. 2023. “The Functional Differentiation of Brain-Computer Interfaces (BCIs) and Its Ethical Implications.” Humanities and Social Sciences Communications 10 (1): 1-9. https://doi.org/10.1057/s41599-023-02419-x.
[49]_Svoboda, Karel, and Nuo Li. 2018. “Neural Mechanisms of Movement Planning: Motor Cortex and Beyond.” Current Opinion in Neurobiology 49 (April): 33-41. https://doi.org/10.1016/j.conb.2017.10.023.
[50]_Van Der Merwe, Anita. 2021. “New Perspectives on Speech Motor Planning and Programming in the Context of the Four-Level Model and Its Implications for Understanding the Pathophysiology Underlying Apraxia of Speech and Other Motor Speech Disorders.” Aphasiology 35 (4): 397-423. https://doi.org/10.1080/02687038.2020.1765306.
[51]_Venezia, Jonathan H., Virginia M. Richards, and Gregory Hickok. 2021. “Speech-Driven Spectrotemporal Receptive Fields Beyond the Auditory Cortex.” Hearing Research 408 (September): 108307. https://doi.org/10.1016/j.heares.2021.108307.
[52]_Willett, Francis R., Erin M. Kunz, Chaofei Fan, Donald T. Avansino, Guy H. Wilson, Eun Young Choi, Foram Kamdar, et al. 2023. “A High-Performance Speech Neuroprosthesis.” Nature, August 1-6. https://doi.org/10.1038/s41586-023-06377-x.
[53]_Wilson, Stephen M, Ayşe Pinar Saygin, Martin I Sereno, and Marco Iacoboni. 2004. “Listening to Speech Activates Motor Areas Involved in Speech Production.” Nature Neuroscience 7 (7): 701-2. https://doi.org/10.1038/nn1263.
[54]_Wilson, Stephen M., Melodie Yen, and Dana K. Eriksson. 2018. “An Adaptive Semantic Matching Paradigm for Reliable and Valid Language Mapping in Individuals with Aphasia.” Human Brain Mapping 39 (8): 3285-3307. https://doi.org/10.1002/hbm.24077.

### Example 4: Bilingual Speech Neuroprosthesis Driven by Multilingual Cortical Articulatory Representations

### 4.1. OVERVIEW

A core goal of speech neuro-prosthetics is to restore naturalistic communication to all people living with paralysis. Over half of the world is bilingual, having proficiency in at least two languages. However, advancements in speech decoding from the brain have focused on monolingual decoding, leaving generalizability to bilinguals an open question. Further, from a more fundamental perspective, the extent to which unique or shared cortical activity underlies bilingual speech production in each language is unclear. This has important implications for training language-specific decoders without multiplying training time for the participant. Here, electrocorticography (ECoG) was leverage to directly record cortical activity on speech-motor cortex from a Spanish-English bilingual with vocal-tract and limb paralysis. Using deep-learning models, along with natural-language models of English and Spanish, cortical activity was flexibly decoded into bilingual sentences without the participant needing to manually select the target language. It is shown that bilingual decoding relies on shared articulatory representations between languages that enable a syllable classifier trained in one language to generalize to shared syllables in the other with no additional training data. Neural data recorded with one language improved decoding in the other language via transfer learning, expediting training of the bilingual decoder. Together, these findings demonstrate a multilingual cortical articulatory representation that persists after paralysis and enables flexible decoding of multiple languages.

### 4.2. INTRODUCTION

Anarthria is a loss of the ability to articulate speech [1] and can be a severe symptom of neurological conditions such as stroke and amyotrophic lateral sclerosis. Invasive speech-based brain-computer interfaces (BCIs) that decode cortical activity into intended speech have great potential to restore naturalistic communication to patients with anarthria and paralysis. Specifically, intracortical electrodes, stereo-electroencephalography (sEEG), and electrocorticography (ECoG), which directly records electrical signals from the cortical surface, can capture neural activity relevant to produced speech [2-9]. However, speech-BCI advancements have largely focused on decoding a single language, primarily English or Dutch, due to study-population sampling [3,5-8,10-15]. A focus on monolingual and English decoding is not unique to the field of speech neuro-prosthetics and parallels trends seen in automatic speech recognition and language modeling. As a result, language technologies for bilingual speakers, as well as speakers of non-English languages, are often less developed [16,17].

Approximately two-thirds of the world is estimated to be bilingual, proficiently speaking two or more languages [18]. Research indicates that the multiple languages an individual speaks serve complementary functions for communication. For instance, bilinguals often report using their languages (L1-native and L2-acquired; in some cases L2 may also be native) in distinct speaker and social contexts and further report that the languages they speak contribute distinct dimensions to their overall personality and worldview [19-22]. To develop a neuroprosthesis capable of restoring embodied communication to all who could benefit, regardless of language background, it is essential to design BCI systems capable of multilingual decoding.

To naturally decode bilingual phrases, it is desirable for the system to flexibly infer the intended language of the participant entirely based on cortical activity and/or natural language models, which capture language-specific word sequence statistics. It is unclear whether intended language can be decoded directly from cortical activity in common speech-motor areas such as the inferior frontal gyrus (IFG, or Broca's area) and the sensorimotor cortex (SMC). A shared articulatory (vocal-tract motor) representation across languages would allow models to generalize rapidly, minimizing required training time and burden on the participant. However, this would present a challenge in decoding the intended language from cortical activity alone.

The extent to which shared representations or language-specific activation patterns exist in speech-motor cortex is unclear. Some evidence suggests that multilingualism may alter core speech-motor networks [21,23,24]. Specifically, learning a non-native second language (L2) may recruit distinct patterns of cortical activity [25-28] or evoke stronger activity in regions of the speech network like the IFG or SMC [29-34]. In support of shared representations between L1 and L2, recent work has shown that the same general anatomical regions tend to be activated across languages [35-38]. Additionally, the brain may fit a non-native L2 into the articulatory framework of L1, forming, for example, shared syllable representations and speech-motor patterns [30,39,40]. Bilingual speech production has primarily been explored with fMRI, leaving an open question for how the precise temporal dynamics underlying shared or distinct cortical representations enable decoding.

Here, ECoG is used to demonstrate a bilingual speech neuroprosthesis in a Spanish-English participant with severe anarthria and paralysis (ClinicalTrials.gov; NCT03698149). During attempted speech, neural activity is flexibly decoded from speech-motor cortex word-by-word into English and Spanish phrases, using a vocabulary of 178 unique words. The intended language is primarily inferred by scoring candidate-decoded sentences with English and Spanish language models, incorporating the differential statistical patterns of word sequences in each language that build through a sentence. Despite the participant learning English later in life, after his stroke, neural activity patterns, particularly those important for decoding, are shared, with no language-specific electrodes. This study demonstrates that these shared activity patterns best represent the articulatory content of speech and facilitate generalization of a syllable classifier across languages. Further, it is shown that performance on a vocabulary in a given language can be improved and expedited by utilizing training data previously collected in the other language, reducing the required time and burden for bilingual participants to utilize all their languages.

### 4.3. RESULTS

### 4.3.1. Performance of the Bilingual Speech Neuroprosthesis

A system capable of flexibly decoding English and Spanish phrases in a participant with paralysis and anarthria due to brainstem stroke was designed (ClinicalTrials.gov; NCT03698149). The models were trained on neural features from a high-density, 128-channel ECoG array primarily covering the left sensorimotor cortex and inferior frontal gyrus.

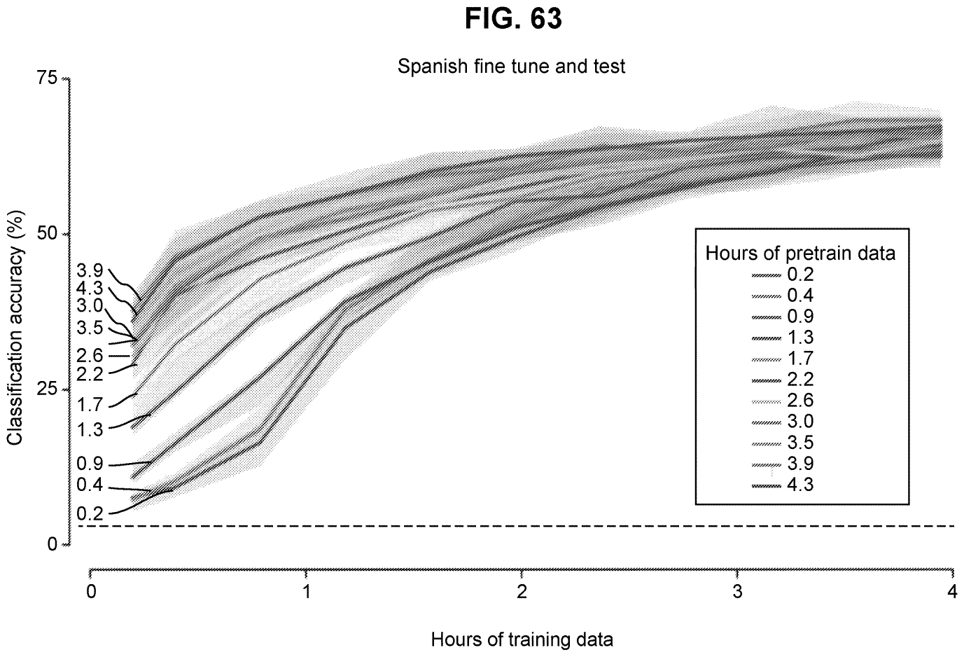

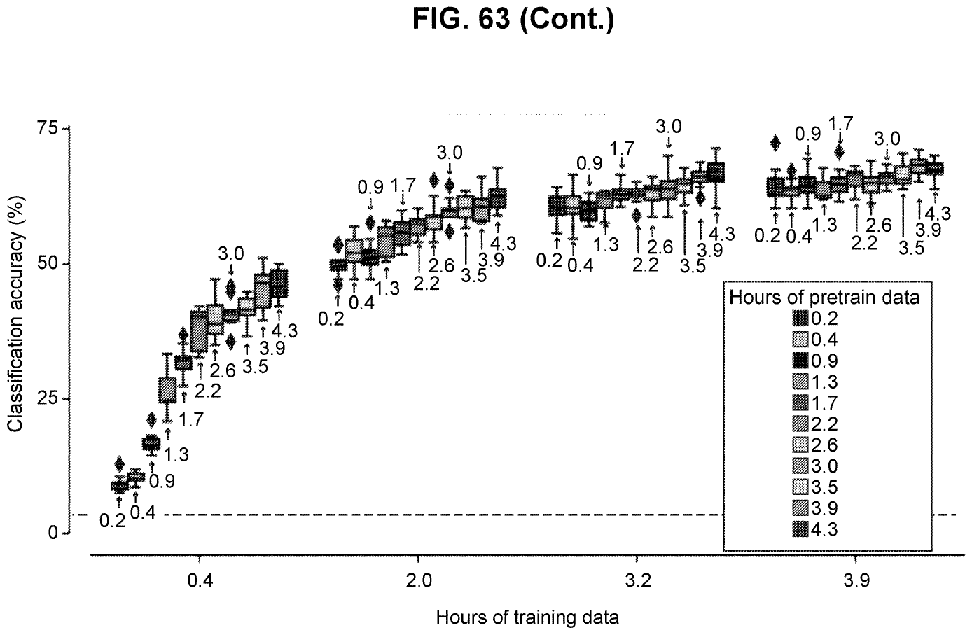

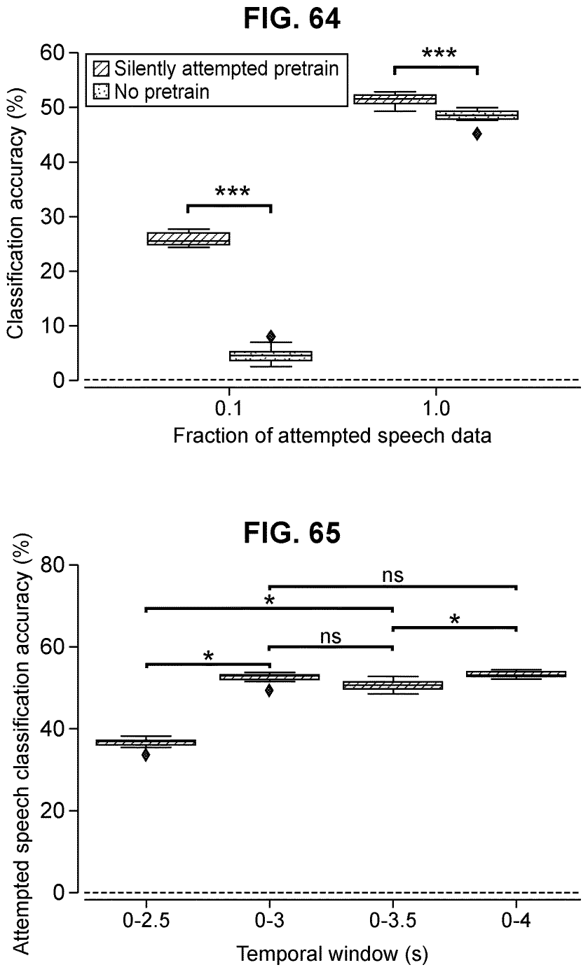

During each phrase-decoding trial, the system displayed a single English or Spanish phrase on the screen as the current target. The phrase-decoding system continuously recorded local field potentials (LFP) from each electrode in the ECoG array and extracted relevant neural features, specifically high-gamma activity (HGA; 70-150 Hz) and low-frequency signals (LFS; 0.3-100 Hz; FIG. 59A; FIG. 63). The participant volitionally activated the system by attempting to speak, and this initial speech attempt was identified by a speech-detection module. Once this initial attempt was detected, the phrase-decoding system activates and presents a series of go cues every 3.5 seconds. In each 3.5-s window, the participant attempted to say a single word. The vocabulary, referred to as bilingual-words, consisted of 51 English words, 50 Spanish words, and 3 words shared between languages (no, a, and the participant's nickname, Pancho; Table 34) for a total of 104 words. Within each cued window, the neural features were streamed to a classifier trained to emit probabilities over the bilingual-words (FIG. 59A; FIG. 64). These probabilities were then split for English and Spanish words. Given that verbs and adjectives, especially in Spanish, may have multiple different conjugations, the predicted probability for the unconjugated form of the verb was broadcasted to all the conjugated forms. For example, the predicted probability for the word “traer” would be broadcasted to the words in the set {traer, traigo, traes, trae, traemos, traen, trayendo}, and the probability for the word “bring” would be broadcasted to the words in the set {bring, brings, bringing}. This led to an increased vocabulary size of 111 Spanish and 70 English words for a total of 178 unique words (67 English, 108 Spanish, and 3 shared; Table 35). A beam search was then applied to combine probabilities with separate unilingual natural-language models trained in English or Spanish. The use of natural-language models prioritizes linguistically valid phrases and properly conjugates verbs based on preceding and following context. A composite score is generated for phrases in each language that reflects a combination of neural predictions and phrase likelihood under the language model (LM). As a final step, an integrator module chooses the highest-scoring phrase across the two languages to display on the screen. As speech attempts were being made by the participant, the speech-detection module continued to predict speech events. The decoding system monitored these events over the course of the trial and deactivated when a speech attempt was not detected in the preceding 3.5s window. The system then advanced to the next trial after a brief delay.

TABLE 34
Bilingual-words used in isolated-target task.

| English | Spanish | English | Spanish | Both |
|---|---|---|---|---|
| store | hermana | i | yo | no |
| nurse | agua | we | nosotros | a |
| sister | almuerzo | you | tu | Pancho |
| water | aquí | your | ella | |
| lunch | ingles | she | ustedes | |
| here | español | they | nos | |
| english | ropa | my | mi | |
| spanish | nombre | not | si | |
| clothes | hablar | yes | por favor | |
| name | estar | if | el | |
| speak | ser | please | la | |
| is | querer | the | ahora | |
| want | tener | now | d [symbol] nde | |
| have | comer | where | y | |
| eat | ir | to | muy | |
| go | traer | and | hola | |
| bring | poder | very | c [symbol] mo | |
| can | gustar | hello | familia | |
| like | divertida | how | sobrino | |
| funny | r [symbol] pidx | family | alimento | |
| fast | reflexivo | nephew | espalda | |
| thoughtful | caliente | food | casa | |
| hot | despierto | back | coche | |
| awake | buenx | house | tienda | |
| good | nuevo | vehicle | enfermero | |
| new | | | | |

[symbol]

indicates data missing or illegible when filed

TABLE 35
Bilingual-words used in phrase-decoding.

| [symbol] | [symbol] | [symbol] | [symbol] | [symbol] | [symbol] | [symbol] | [symbol] |
|---|---|---|---|---|---|---|---|
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | [symbol] | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | [symbol] | [symbol] | | [symbol] | [symbol] |
| [symbol] | | [symbol] | | [symbol] | | [symbol] | [symbol] |
| [symbol] | | [symbol] | | [symbol] | | [symbol] | [symbol] |
| [symbol] | | [symbol] | | [symbol] | | [symbol] | [symbol] |
| [symbol] | | [symbol] | | [symbol] | | [symbol] | [symbol] |
| [symbol] | | [symbol] | | [symbol] | | [symbol] | [symbol] |
| [symbol] | | [symbol] | | [symbol] | | [symbol] | [symbol] |
| [symbol] | | [symbol] | | | | | |
| [symbol] | | [symbol] | | | | | |
| [symbol] | | [symbol] | | | | | |
| [symbol] | | [symbol] | | | | | |
| [symbol] | | [symbol] | | | | | |
| [symbol] | | [symbol] | | | | | |

[symbol]

indicates data missing or illegible when filed

Classification and detection models were trained prior to phrase decoding with an isolated-target task. In this task, a single bilingual-word was presented on the screen, and the participant attempted to produce the target word at a visual go cue. The HGA and LFS features spanning from the go cue to 3.5s after the go cue were used to predict the target bilingual-word.

The decoding pipeline was evaluated using a similar copy-typing task to past work [13]. The participant was prompted with randomly interleaved English and Spanish phrases, which he tried to replicate (Video 1). During evaluation, decoding models were fit using data exclusively from preceding sessions with no day-of recalibration. To measure performance, the word error rate (WER) metric was primarily used, commonly used to evaluate outputs of automatic speech recognition systems and communication BCIs [13,41-43]. 3 repetitions of 56 phrases were collected (split between English and Spanish, Table 36), covering all unconjugated bilingual-words across languages. Median word error rates were achieved across online testing blocks of 25.0% (99% CI: [17.2, 36.4] %) on all phrases, 26.7% (99% CI: [18.2, 33.3] %) for Spanish phrases and 22.2% (99% CI: [7.14, 44.4] %) for English phrases (FIG. 59B). Decoding with neural data alone, without language modeling or beam search, achieved median word error rates of 70.6% (99% CI: [61.9, 78.1] %) overall, 52.5% (99% CI: [40.4, 61.7] %) for Spanish phrases and 55.0% (99% CI: [46.3, 68.8] %) for English phrases (FIG. 59B), indicating that decoding did not only depend on the use of LMs (see FIG. 65 for a comparison to chance neural-only performance). Performance on all phrases as well as separately on English and Spanish phrases, using either the full-system or neural-only decoding, was significantly better than chance (full-system performance with temporally shuffled neural data; Table 37). During online testing median language-classification accuracy of 87.5% (99% CI: [85.7, 100.] %) was achieved, freely decoding intended language based on the overall highest-scoring phrases. This was significantly better than both chance predictions and picking language based on neural activity alone (product of highest probability words in each language), illustrating the importance of language modeling in choosing the correct language (FIG. 59C, Table 38).

TABLE 36
Copy-typing task sentences.

| English sentences | Spanish sentences |
|---|---|
| bring back the water | ahora ustedes van |
| can you please go | aquí está la comida |
| eat your lunch | c [symbol] mo está mi espalda |
| hello are you awake | c [symbol] mo está mi hermana |
| hello how are you | dónde está la tíenda |
| hello my name is pancho | el alimento es bueno |
| here we go | el almuerzo es caliente |
| i have a nephew | el coche es rápido |
| i like my new clothes | el enfermero es reflexive |
| i like your family | ella quiere ir a casa |
| i speak english and spanish | hola mi nombre es pancho |
| my family speaks spanish | mi familia esta aquí |
| my family wants lunch | mi hermana es muy divertida |
| my nurse is bringing water | mi sobrino tiene el poder |
| no not now | nosotros estamos comiendo |
| please bring hot water | nosotros nos gusta comer |
| please have my back | por favor trae el alimento |
| she goes to the store | sí yo puedo hablar |
| the house is new | sí yo qu [symbol] ero agua |
| the nurse is thoughtful | trae ropa nueva |
| the store has good food | t [symbol] puedes ir ahora |
| the vehicle is fast | yo estoy despierto |
| they like the food | yo estoy hablando español |
| where is my nephew | yo hablo espa [symbol] ol y ingles |
| where is the food | yo no tengo |
| yes i can speak | yo puedo comer ahora |
| yes if you want | yo quiero agua caliente |
| your sister is very funny | yo quiero comer |

[symbol]

indicates data missing or illegible when filed

TABLE 37
Statistical comparisons of word error rates
across languages and decoding paradigms.

| | | | Test- | P-value |
|---|---|---|---|---|
| Language¹ | Paradigm 1 | Paradigm 2 | statistic | (corrected)² |
| Overall | Chance | Neural-only | 4.41e+02 | 1.91e−07 |
| Overall | Chance | Real-time | 4.41e+02 | 1.91e−07 |
| Overall | Neural-only | Real-time | 4.40e+02 | 1.91e−07 |
| Spanish | Chance | Neural-only | 4.41e+02 | 1.91e−07 |
| Spanish | Chance | Real-time | 4.41e+02 | 1.91e−07 |
| Spanish | Neural-only | Real-time | 4.13e+02 | 2.66e−06 |
| English | Chance | Neural-only | 4.40e+02 | 1.9te−07 |
| English | Chance | Real-time | 4.41e+02 | 1.91e−07 |
| English | Neural-only | Real-time | 3.74e+02 | 1.24e−04 |

¹Each comparison is a two-sided Mann-Whitney U-test with 9-way Holm-Bonferroni correction for multiple comparisons across 21 real-time sentence-decoding blocks.
²9-way Holm-Bonferroni correction for multiple comparisons.

TABLE 38
Statistical comparisons of language classification
accuracy across decoding-paradigms.

| | | | P-value |
|---|---|---|---|
| Paradigm 1¹ | Paradigm 2 | Test-statistic | (corrected)² |
| Chance | Neural-only | 1.05e+02 | 4.79e−03 |
| Chance | Real-time | 1.50e+01 | 5.44e−07 |
| Neural-only | Real-time | 1.02e+02 | 4.79e−03 |

¹Each comparison is a two-sided Mann-Whitney U-test with 3-way Holm- Bonferroni correction for multiple comparisons across 21 real-time sentence- decoding blocks.
²3-way Holm-Bonferroni correction for multiple comparisons.

Selecting the target language (L_target) relied on scoring sequences of decoded words in each language based on their likelihood under neural-classification models and language-specific LMs. While the neural classifier did confuse words in L_target as words in L_other (details discussed in later sections), it was hypothesized that these confusions would not form linguistically likely sequences of words, in contrast to word-sequence predictions in L_target. This difference in linguistic likelihood between L_target and L_other is referred to as differential linguistic context. Differential linguistic context builds throughout a phrase as longer sequences of words can be better scored for linguistic likelihood; correspondingly, language-decoding accuracy improved as a function of position within a phrase, with 100% classification by word 5 versus chance classification at position 1 (FIG. 59E). GPT2, a large neural-network L_{M}, was used to score the linguistic likelihood of the final decoded word sequences in L_target and L_other across trials [44]. For trials in which the language was correctly decoded, sequences in L_target had a significantly higher likelihood than L_other, but, on language-error trials, differential linguistic context did not distinguish L_target and L_other (FIG. 59F). Lack of differential linguistic context on language-error trials could stem from lower likelihoods in L_target or higher likelihoods in L_other. To assess these possibilities, likelihoods were compared for L_target on correct trials with likelihoods for L_target and L_other on incorrect trials. No statistically significant differences were found, implying that likelihood of L_other is increased on incorrect trials, reducing differential linguistic context. This may stem from neural confusions in L_other that form plausible sequences of words. Together, these analyses (FIGS. 59E and 59F) implicate differential linguistic context between languages as a driving force in decoding the target language.

Offline, the performance of the system was simulated when the target language was manually set rather than freely chosen (FIG. 59G; Table 39). Improved word error rates were seen, with a median of 21.9% (99% CI: [16.7, 27.6] %) for all phrases, 20.0% (99% CI: [16.7, 28.6] %) for Spanish phrases, and 20.0% (99% CI: [6.67, 33.3] %) for English phrases.

TABLE 39
Statistical comparisons of word error rates
with manual setting of the target language.

| | | | P-value |
|---|---|---|---|
| Paradigm 1¹ | Paradigm 2 | Test-statistic | (corrected)² |
| Chance | Neural-only | 1.05e+02 | 4.79e−03 |
| Chance | Real-time | 1.50e+01 | 5.44e−07 |
| Neural-only | Real-time | 1.02e+02 | 4.79e−03 |

¹Each comparison is a two-sided Mann-Whitney U-test with 3-way Holm-Bonferroni correction for multiple comparisons across 21 real-time sentence-decoding blocks.
²3-way Holm-Bonferroni correction for multiple comparisons.

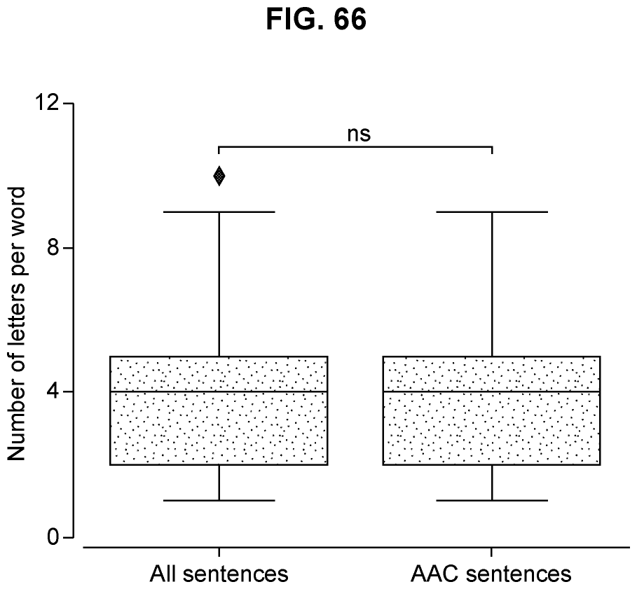

Finally, it is demonstrated that the participant could use the system to openly generate desired phrases from the bilingual-words vocabulary and participate in a conversation where he can fluidly switch between languages based on preference (Video 2). In line with this result, it is verified that neural features were specific to attempted speech and not listening or reading (FIG. 66).

FIGS. 59A-59G: Implementation of a bilingual speech neuroprosthesis. FIG. 59A: Schematic diagram of the bilingual decoding system. In each trial, the participant is presented with a target phrase in English or Spanish. The participant volitionally activates the system by attempting to speak, and this attempt is identified from the neural features by a speech-detection model. After an initial attempted speech event is detected, the system cues the participant to attempt to say the next word in the sequence every 3.5 s. The neural features from each window are processed by a classifier, composed of recurrent neural network (RNN) layers and a fully-connected dense layer, to produce a probability distribution over the 104 possible words across both languages (51 English, 50 Spanish, and 3 shared). The probability vectors over English and Spanish words are processed separately. Here, the neural probability for a verb or adjective in the un-conjugated form is broadcast to all conjugated forms giving a total of 178 unique words (67 English, 108 Spanish, and 3 shared) to be scored by the n-gram language model. The most likely phrase at the end of each window is chosen across languages and displayed to the participant. The system is deactivated when a speech attempt is not detected within a 3.5-s window. FIG. 59B: Word error rates with the phrase test set, calculated using shuffled neural data (Chance), neural decoding from the RNN without language modeling (Neural-only), and the full online system with language modeling (Online) (****P<0.0001, ***P<0.001, See Table 37 for exact p-values; two-sided Mann-Whitney U-test with 15-way Holm-Bonferroni correction for multiple comparisons). FIG. 59C: Language classification accuracy for chance, neural-only, and online results (****P<0.0001, **P<0.005, See Table 38 for exact p-values; two-sided Mann-Whitney U-test with 3-way Holm-Bonferroni correction for multiple comparisons). FIG. 59D: The decoding rate (words per minute) compared to the participant's communication speed with his alternative augmentative communication (AAC) strategy. FIG. 59E: The language-classification accuracy as a function of word position in a phrase. Error bars denote 99% confidence intervals. FIG. 59F: Phrase likelihood scores from GPT2 (large language model) for trials where the language is correctly and incorrectly classified. For each trial, a score is computed for the phrase decoded by the system in the target and off-target languages (****P<0.0001; two-sided Wilocxon Signed-rank test). FIG. 59G: Word error rates, as in FIG. 59B when the target language is manually set rather than freely decoded (****P<0.0001, See Table 39 for exact p-values; two-sided Mann-Whitney U-test with 6-way Holm-Bonferroni correction for multiple comparisons). In FIGS. 59B-59C, 59E and 59G distributions are over 21 online phrase-decoding blocks. Box plots in all figures depict median (horizontal line inside box), 25th and 75th percentiles (box), 25th and 75th percentiles+/−1.5 times the interquartile range (whiskers), and outliers (diamonds).

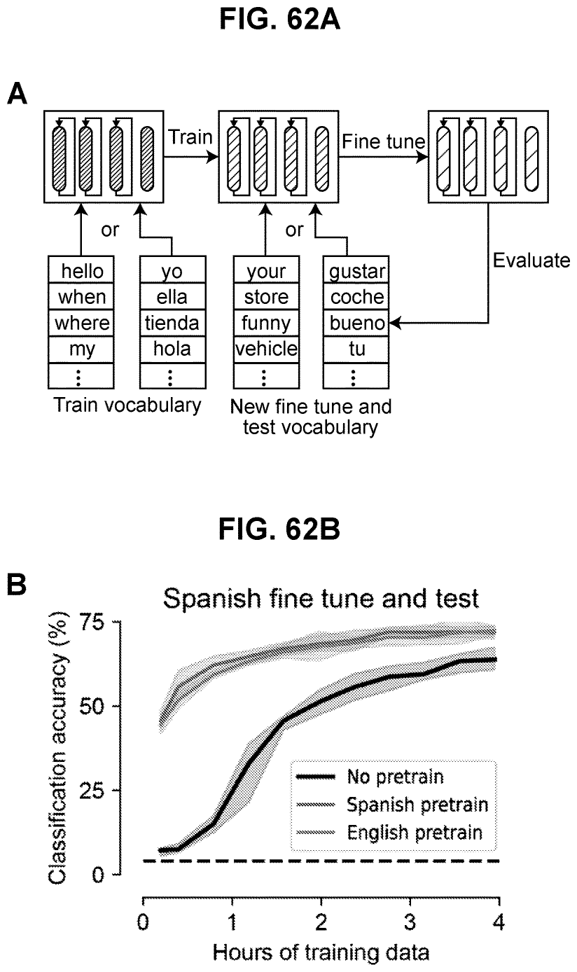

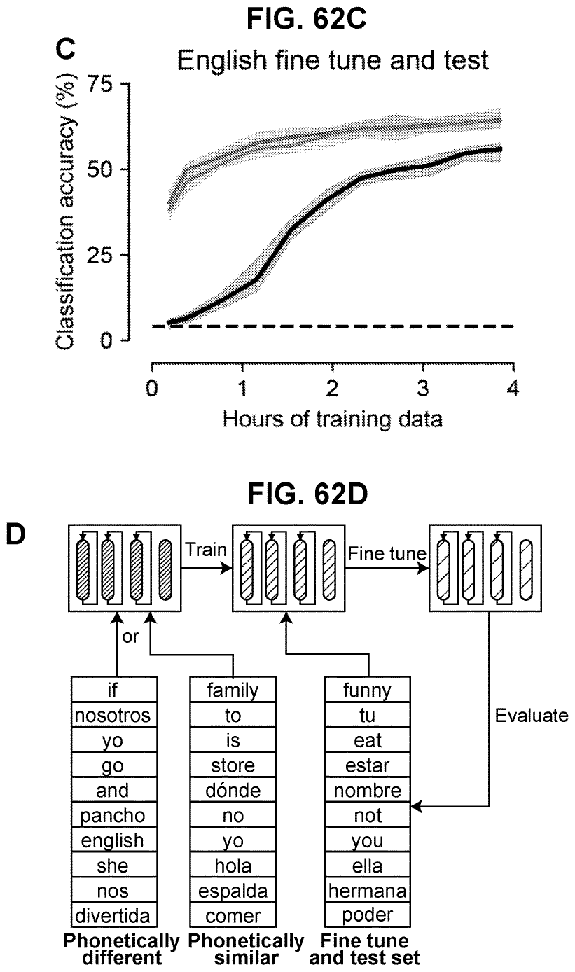

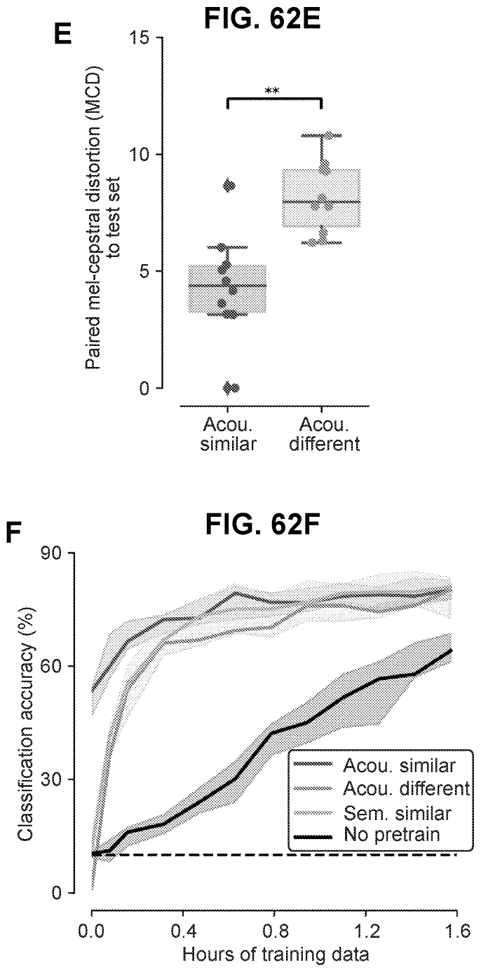

FIG. 63: Effect of amount of pretrain data on transfer learning efficacy. (top) Shown are learning curves on the Spanish vocabulary (FIG. 62B) with transfer learning from English models trained on varying amounts of data. The number of hours of pretraining data has an effect on transfer learning efficacy; however, this effect saturates. Lines represent median accuracy and shaded error bars are 99% confidence intervals. (bottom) Shown is the classification accuracy at select amounts of training data, again as a function of the hours of data used to pretrain the English models. The distribution of accuracy across hours of pretraining data is easier to visualize. Distributions in both panels are over 10 non-overlapping folds. Box plots in all panels depict median (horizontal line inside box), 25th and 75th percentiles (box), 25th and 75th percentiles+/−1.5 times the interquartile range (whiskers), and outliers (diamonds).

FIG. 64: Effect of pre-training with silently attempted speech data. Shown is performance of classification models, over the full 104 bilingual words, at two fractions of total attempted speech data: 0.1 and 1. At both fractions, pre-training with the limited silently attempted speech data helps improve performance, albeit marginally with full use of the attempted speech data (Median 21.00% and 3.12% improvement at 0.1 and 1, respectively, ***P=0.00018 and P=0.00044, two-sided Mann-Whitney U-test). Dashed line indicates chance performance (0.96%). Box plots in all panels depict median (horizontal line inside box), 25th and 75th percentiles (box), 25th and 75th percentiles+/−1.5 times the interquartile range (whiskers), and outliers (diamonds).

FIG. 65: Performance of attempted speech model using windows of various input lengths. Classification performance was evaluated over the full 104 bilingual words, using models trained with varying length temporal windows of neural features. Classification training and evaluation occurred identically to described in the methodology and each distribution is over 10-fold cross validation (*P<0.01; two-sided Mann-Whitney U-test with 6-way Bonferroni correction for multiple comparisons). For 0-2.5 vs 0-3, 0-3 vs 0-3.5, 0.35 vs 0.4, 0-2.5 vs 0-35, 0-3 vs 0-4, and 0-2.5 vs 0-4, p-values are 0.0011, 0.1, 0.0019, 0.0011, 0.32, and 0.0011. Dashed line indicates chance performance (0.96%). Box plots in all panels depict median (horizontal line inside box), 25th and 75th percentiles (box), 25th and 75th percentiles+/−1.5 times the interquartile range (whiskers), and outliers (diamonds).

FIG. 66: Comparison of all online-evaluation sentences and AAC evaluation subset. For each word in each of the online-evaluation sentences, the number of letters was computed. The same was done for the 30 random sentences drawn for the AAC evaluation. Distributions are over 126 words in the AAC sentences and 227 words in the evaluation sentences with no significant difference (NS P=0.71, two-sided Mann-Whitney U-test). Box plots in all panels depict median (horizontal line inside box), 25th and 75th percentiles (box), 25th and 75th percentiles+/−1.5 times the interquartile range (whiskers), and outliers (diamonds).

### 4.3.2. Offline Characterizations of Neural-Decoding Performance

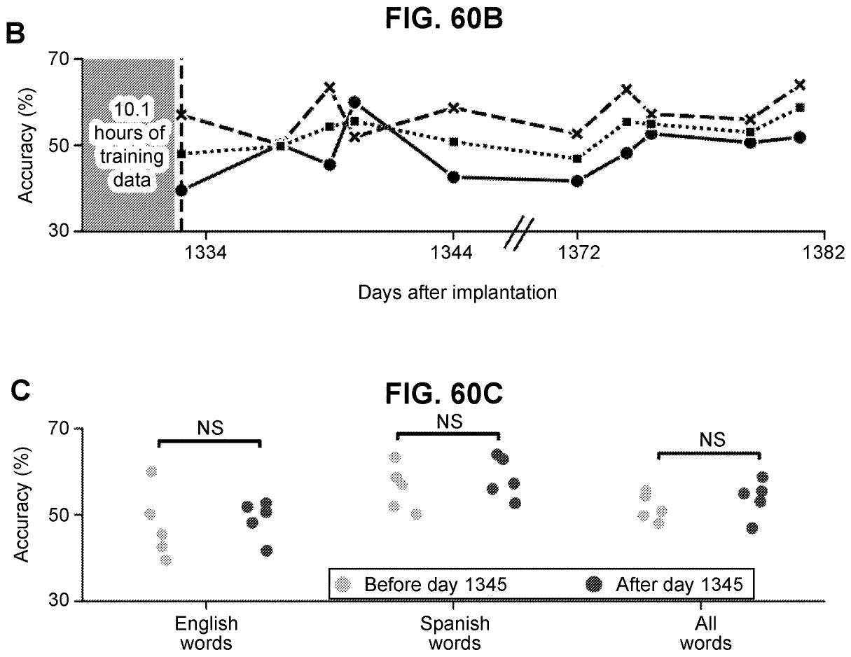

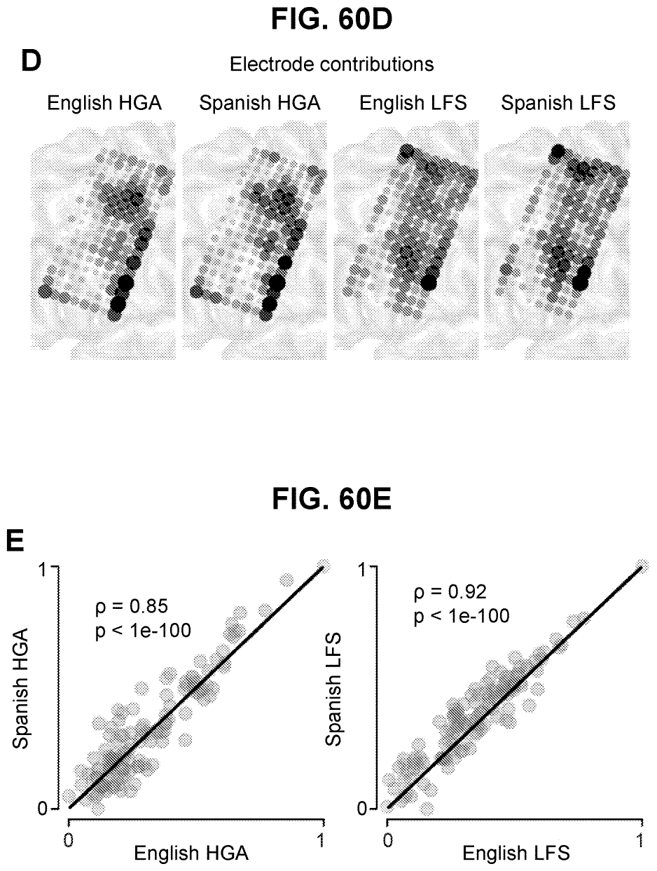

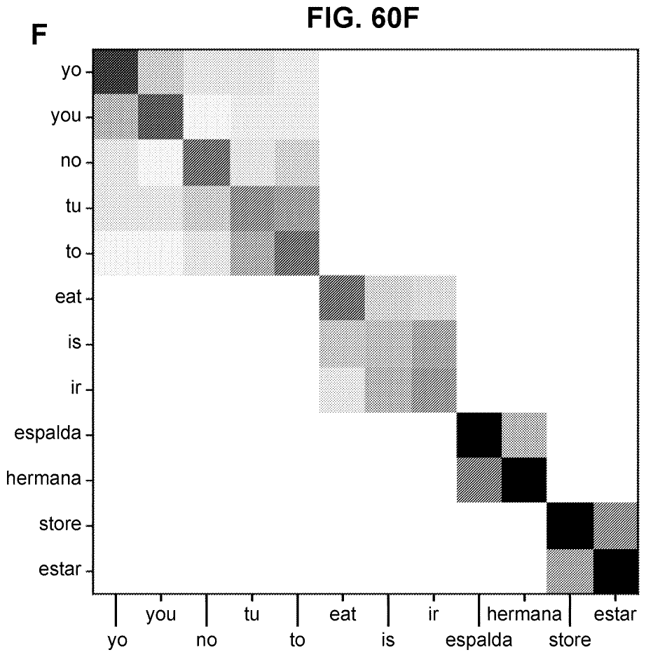

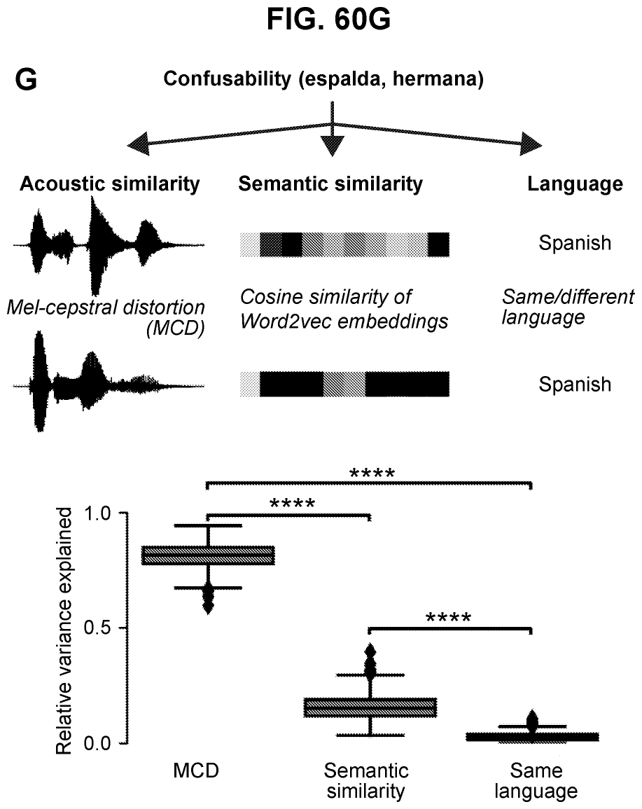

To further characterize the system's ability to decode words in both English and Spanish from neural features, 10-fold cross validation (CV) was used to evaluate classification performance on the isolated-target data collected prior to phrase decoding. A classification model was trained on bilingual-words across both languages, but the predictions were masked in the off-target language for each word during training and testing (see Methods). During training, this encourages the model to learn predictions consistent with the vocabulary in each language. During evaluation, this approach allowed the performance of the model on English, Spanish, and combined vocabularies to be probed. Median CV classification accuracies of 58.1% (99% CI: [56.9, 59.3] %) overall, 62.9% (99% CI: [61.3, 64.9] %) for Spanish words, and 52.9% (99% CI: [51.6, 55.6] %) for English words were achieved (FIG. 60A). Median CV classification accuracy was also computed over the full 104-word vocabulary (no masking), which was 47.2% (99% CI: [45.8, 48.2] %). Differences in English and Spanish classification accuracy may stem from characteristics of the specific stimuli used.

An important challenge in developing clinically viable BCIs is maintaining similar decoding performance day-to-day without requiring the user to dedicate time to frequently recalibrate the system. The relatively large spatial-sampling scale of ECoG offers the potential for consistent day-to-day signal acquisition [45-49]. Notably, it was found that the models were able to maintain similar classification accuracy, with some day-to-day variance, for 48 days without recalibration (FIGS. 60B and 60C). This highlights that similar modulation patterns for each word in the neural features are maintained over 48 days. These results were achieved over 3.5 years after ECoG-device implantation with improved classification performance on a slightly larger vocabulary than initial work with this participant [13], demonstrating longevity of speech-information content in the neural signals. Modest improvements in performance were possible with adding more data to the models by re-training each day.

Given evidence in the literature that L1 and L2 may activate distinct cortical regions [26,28], it was next examined whether classification models trained only on English or Spanish words utilized different electrodes or features. It was found that for both the HGA and LES feature types, electrode contributions to the classifier (see Methods) were similar between English and Spanish (FIG. 60D). Indeed, non-parametric correlation between electrode contributions for English and Spanish, for both HGA (P<0.0001, p=0.85, non-parametric correlation permutation test; FIG. 60D) and LFS (P<0.0001, p=0.92, non-parametric correlation permutation test; FIG. 60E), showed a strong positive relationship. In contrast, for both English and Spanish, very few electrodes contributed strongly with both LFS and HGA, indicating complementary information in the two bands.

Based on this result, along with array coverage of the sensorimotor cortex, it was hypothesized that shared articulatory representations were driving decoding [2,50,51]. To assess this, a model trained on all 104 bilingual-words was used (no masking) and which factors drove confusability between any given pair of words was explored. Here, confusability refers to the number of times that a given target word was incorrectly classified as another word in the dataset. Specifically, the effect of the following three similarity measures on confusability was assessed: 1) semantic similarity, measured by high-dimensional Word2Vec embeddings [52]; 2) acoustic similarity, measured by mel-cepstral distortion (MCD) [53]; and 3) whether the pair of words were in the same language (FIG. 60G). A multiple-regression model was fitted to predict confusability between each pair of words from these three variables. It was found that acoustic similarity between words had significantly stronger relative explanatory power than semantic similarity or whether the pair of words was in the same language (FIG. 60G; P<0.0001, two-sided Mann-Whitney U-test with 3-way Holm-Bonferroni correction). Given that acoustics are a strong proximate measure for articulation [2,54], this provides evidence that the classification models capture shared articulatory information at the same electrode sites rather than language-specific signals.

FIGS. 60A-60G: Offline characterizations of the bilingual classification algorithms. FIG. 60A: 10-fold cross-validation classification accuracy for English words, Spanish words, and across both languages (all words). FIG. 60B: Classification of words in English, Spanish, and across both languages for 48 days without retraining or recalibration of the system. Classifiers were trained from data collected over a 50-day period and then the weights were frozen (black dashed line). Results were achieved over 3.5 years after ECoG-device implantation. The break in the axis indicates a 30-day break in recording sessions with the participant. No retraining occurred between sessions separated by the 30 day break. FIG. 60C: Classification performance before (n=5 days) and after (n=5 days) a 30-day break in recording without retraining. FIG. 60D: Electrode contributions for models trained only on English or Spanish words, separated by neural-feature type (high-gamma amplitude (HGA); low-frequency signal (LFS)). FIG. 60E: Relationship between 128 HGA (left) and LFS (right) electrode contributions for Spanish and English models. Correlation assessed with non-parametric Spearman rank correlation and permutation test. FIG. 60F: Selected portion of the confusion matrix between bilingual words, highlighting confusability. FIG. 60G: Multiple regression models were fit to predict confusability between a pair of words from their acoustic similarity (measured by Mel-cepstral distortion; MCD), semantic similarity (measured by cosine similarity of Word2Vec embeddings), and whether the words are in the same language (top). The relative variance explained by each factor in the multiple regression model (bottom). Distributions were created by bootstrapping the confusion matrix with replacement 2000 times. ****P<0.0001, two-sided Wilcoxon Signed-Rank test with 3-way Holm-Bonferroni correction for multiple comparisons. Box plots in all panels depict median (horizontal line inside box), 25th and 75th percentiles (box), 25th and 75th percentiles+/−1.5 times the interquartile range (whiskers), and outliers (diamonds).

### 4.3.3. a Shared Cortical Representation of English and Spanish Phrases in Speech-Motor Cortex

The neural representation of English and Spanish speech was next directly probed across the participant's electrode array in a model-agnostic manner. A large set of unique phrases with around 200 words in each language was designed (FIG. 61A) to sample a larger articulatory space in each language (Table 40). This allowed whether the magnitude or localization of neural activity was different between languages to be evaluated in a less biased manner. The standard deviation of the average HGA (0-2 seconds after the go cue) was first computed across English and Spanish phrases for each electrode and the values on the cortex were visualized (FIG. 61B). Sample evoked response potentials (ERPs) are shown for two electrodes, demonstrating temporally similar neural-activity patterns for English and Spanish phrases (FIG. 61C). Next, 2 key metrics were quantitatively compared across electrodes and between languages. A strong positive correlation of the maximum of the HGA ERP (P<0.0001, ρ=0.77, spearman correlation permutation test; FIG. 61D) and standard deviation of the average HGA (described above, P<0.0001, ρ=0.95, spearman correlation permutation test; FIG. 61E) between languages was found.

TABLE 40
Large bilingual phrase set.

| English sentences | English sentences (cont) | Spanish sentences | Spanish sentences (cont) |
|---|---|---|---|
| stop that hurts | they went outside | soy gracioso | me gusta al pastel |
| you like pizza | how do you write | hola como estás | no ahora no |
| this weekend is free | on my way | no puedo dormir | usa un casco |
| wear a jacket | use a helmet | por favor dile a mi familia | estoy impresionado |
| call me back | my uncle is fun | por dentro es c [symbol] lida | por qu [symbol] hace frio |
| do me a favor | swim in the river | ellos corrieron lento | conduce mi coche |
| you bike quickly | you seem confused | tenedor o cuchara | cuàndo esta aqui |
| look it is snowing | we play outside | todos ustedes son amables | tras a tus amigos |
| your house is large | pen or pencil | estas enfermo | las c [symbol] udades est [symbol] n ocupadas |
| chicken is tasty | she is on vacation | mi tio es divertido | cierra la ventana |
| he walks fast | trees are green | si entra | azul es un color |
| they eat soup | your sister is tall | puedo cir | llama a tu hermano |
| is the water clean | is it raining | te gusta la pizza | este fin de semana está libre |
| dinner is ready | let me see | tu hermana es alta | [symbol] una mano |
| they ran fast | fruits or vegetables | esta es mi casa | frutas o verduras |
| my back hurts | go to your room | dame un segundo | tu casa es grande |
| drive my car | you all are nice | mira est [symbol] nevando | nadar en el rio |
| faith is good | i am impressed | camisa roja y pantalones | estoy emocionado |
| close the door | sleep with a pillow | pasea al perro | su pelo es largo |
| shut the window | cities are busy | el pollo es sabroso | avi [symbol] n tren o autob [symbol] s |
| yes come in | i like cake | tras mis lentes | los perros son lindos |
| do you like music | blue is a color | ellos comen sopa | te gusta la [symbol] |
| give me a hand | i can hear | para eso me duele | esto es c [symbol] modo |
| we are lucky | hello how are you | como escr [symbol] bes | déjame ver |
| why is it cold | where did you go | me duele la espalda | no [symbol] preocupes demasiado |
| cats are shy | bring my pillow | aqu [symbol] vamos | tiralo a la basura |
| is that your wife | how will you feel | la computadora est [symbol] apagada | duerme con una almohada |
| against the law | are you sick | huevos y tostadas | cierra la puerta |
| the coffee is perfect | when is it here | la le es buena | nosotros [symbol] |
| how is your heart | bring your friends | [symbol] o l [symbol] piz | ella est [symbol] de vacaciones |
| no not now | i enjoy sports | està lloviendo | [symbol] camina rápido |
| it is comfortable | i am funny | hazme un favor | ahora tengo hambre |
| please tell my family | let's have fun | vaso de lache | cuando debemos ir |
| this is my home | eat a banana | c [symbol] mo te [symbol] | los [symbol] son verdes |
| give me a second | yesterday i left | tenemos suerta | esto asté limpio |
| throw it away | i received the package | el cielo est [symbol] nublado | adonde fuiste |
| the road is wet | red shirt and pants | el camino est [symbol] mojado | [symbol] por favor |
| the computer is off | i like my nurse | me gusta mi enfermera | andas en bicicleta rápido |
| walk the dog | turn on the television | c [symbol] mo está tu corazón | esa es tu esposa |
| i am excited | plane train or bus | leo libros | [symbol] confundido |
| dogs are cute | come here please | disfruto de los deportes | ven aquí por favor |
| now i am hungry | when should we leave | el agua est [symbol] limpia | la cena est [symbol] lista |
| i can't sleep | bring my glasses | ellas [symbol] | estoy en camino |
| call your brother | it is clean | vamos a divertimos | ayer me fui |
| cup of tea | inside in warm | tras mi almohada | taza de [symbol] |
| don't worry too much | hurry home please | come una banana | usar una chaqueta |
| fork or spoon | glass of milk | ap [symbol] rate a casa por favor | va a tu cuarto |
| here we go | what a catch | [symbol] el paquete | los gatos son timidos |
| her hair is long | the sky is cloudy | vaya trampa | enciende et televisor |
| i read books | eggs and toast | s [symbol] café está perfecto | contra la ley |

[symbol]

indicates data missing or illegible when filed

To better understand if the temporal dynamics of neural responses differed between languages, HGA ERPs computed from trials in the same language were correlated with ERPs computed from trials in the other language. Again, a strong positive relationship between correlations within and between languages across electrodes was found (P<0.0001, p=0.98, non-parametric correlation permutation test; FIG. 61F). Finally, a deep-learning model that predicted whether a phrase was English or Spanish based on the neural features, HGA and LFS, during the speech attempt was trained. 53.3% median 10-fold CV classification accuracy (FIG. 61G; 99% CI: [49.0, 55.8] %) was achieved, indicating that performance was not different from chance. Therefore, across a large phrase set, the precise temporal patterns of neural features in speech-motor cortex also cannot strongly distinguish between languages.

FIGS. 61A-61J: A shared articulatory representation in speech-motor cortex across languages. FIG. 61A: Large stimulus set of unique words and phrases used to cover a larger articulatory space in each language, relative to the vocabulary used for core phrase-decoding (FIG. 59). FIG. 61B: Standard deviation of the average high-gamma amplitude (HGA) from 0 to 2 seconds (relative to the visual go cue) for English and Spanish phrases across each electrode. FIG. 61C: Sample evoked response potentials (ERPs) to English and Spanish phrases for two electrodes noted in FIG. 61B. FIG. 61D: Relationship between the maximum HGA for each electrode during English and Spanish phrases. FIG. 61E: Relationship between the HGA standard deviation (as in FIG. 61B) for English and Spanish phrases for each electrode. FIG. 61F: Relationship of the correlation of ERPs within a language to the correlation of ERPs between languages for each electrode. FIGS. 61D-61F: correlation assessed with non parametric Spearman rank correlation and permutation testing over 128 electrodes. Values normalized to fall between 0 and 1. FIG. 61G: 10-fold cross-validation (CV) classification accuracy for classifying each phrase as English or Spanish. FIG. 61H: Stimulus set designed to probe a shared articulatory, syllabic representation between languages. Classifiers were trained in each language and tested in both the same and the other language. FIG. 61I: Sample ERPs from an electrode indicated in FIG. 61B for different syllables in the same language and a shared syllable in different languages. FIG. 61J: 10-fold CV syllable-classification accuracy across training and testing paradigms. Two-sided Mann-Whitney U-test (NS: P>0.01). FIGS. 61A and 61I Shaded regions indicate the standard error of the mean at each timepoint, computed across trials.

### 4.3.4. Shared Syllable Representations Enable Cross-Language Training and Testing of Classifiers

It is hypothesized that bilinguals who learn L2 later in life may fit the articulatory content of L2 into previously learned L1 representations. One way this may manifest is in a conserved syllable representation between languages [30,39,40]. Given the strong evidence for shared articulatory representations with the participant, whether a syllable classifier could generalize between English and Spanish was assessed. Aan utterance set was designed in which the same 7 syllables were present in 7 English and Spanish words (FIG. 61H; Table 41). ERPs from a sample electrode demonstrate a clear similarity in neural activity for the same syllable across languages (FIG. 61I). Next, syllable-classification models were trained over the shared syllable set using neural data recorded as the participant attempted to say the English or Spanish words. These models were evaluated either on held-out data from the same language or data from the other language. The syllable classifier achieved high performance regardless of whether training and testing occurred in the same language (FIG. 61J; P>0.01, two-sided Mann-Whitney U-test, for train English (Spanish) and test Spanish (English)). This provides compelling evidence that a shared syllable representation can allow data collected in one language to be repurposed for a second language.

TABLE 41
English and Spanish syllable paired words.

| English words | Spanish words | Shared syllable |
|---|---|---|
| boulder | bolsa | Bol |
| casket | castor | Cas |
| crazy | crece | cre/crae |
| petal | pesar | Pe |
| soda | fonda | Da |
| soda | solas | So |
| widen | pierden | den |

### 4.3.5. Rapid Transfer Learning Between Languages

Transfer learning is a common technique in machine learning that involves the initialization of model weights with parameters learned on a separate task or dataset [55,56]. Transfer learning has primarily been used in neural decoding to leverage models trained in prior participants to expedite training in a new participant [57,58].

Given strong shared articulatory representations between languages, it was hypothesized that transfer learning between different languages in the same participant should expedite learning a new vocabulary. To assess this, a potential use case was evaluated where models trained on a vocabulary in a given language were used to learn a new vocabulary in a second language. This performance was benchmarked against a scheme in which models trained on a vocabulary in a given language were used to learn a new vocabulary in the same language. English and Spanish models were pre-trained on 4.33 hours of data from a vocabulary of 25 words in each respective language. These models were then fine-tuned and tested on a new set of 25 Spanish words (FIG. 62B). Interestingly, it was found that pre-training on an English vocabulary achieved equivalent performance to pre-training in Spanish. Further, significantly higher performance was achieved on the new vocabulary using either pre-training scheme, compared to no pre-training, after just minutes of new training data. An analogous outcome was observed when pre-trained models were fine-tuned and tested on a new set of 25 English words (FIG. 62C). Overall, this demonstrates that rapid decoder training with a vocabulary in a new language can be achieved by leveraging prior data collected in a different language, minimizing required training times for multilingual BCI use.

To further explore the factors that drove transfer learning and whether models could generalize to an entirely new vocabulary, with no additional training data, a new 10-word fine-tune and test set was designed (FIG. 62D). Next, three pre-training sets were designed by choosing a word to pair with each of the 10 words in the test set that was either acoustically similar, acoustically (acou.) different, or semantically (sem.) similar (FIG. 62D). It was verified that the pairwise MCD between each word in the acoustically similar set and the corresponding word in the test set was significantly lower than that of the acoustically different set and the test set (FIG. 62E; P<0.005; two-sided Wilocxon Signed-rank test). Learning curves were then computed on the new test set, using transfer learning from one of the three pre-training sets or no transfer learning (FIG. 62F). Here an evaluation point at 0 hours of training data was also included, which indicates the ability of the pre-trained model to generalize with no additional data specific to the fine-tune set. Notably, it was only seen in the case of an acoustically similar pre-training set is this possible, and learning curves with the acoustically similar set increase most rapidly (FIG. 62F). Overall, this demonstrates that careful design of pre-training and fine-tune/test sets for acoustic, and by virtue articulatory [2,54], similarity may allow generalization of models to bilingual vocabularies with as little as no additional training data.

FIGS. 62A-62F: Rapid transfer learning between languages. FIG. 62A: Schematic depiction of the paradigm used to evaluate transfer learning between languages. Models were trained on an English or Spanish vocabulary. These models were then fine-tuned and evaluated on a new English or Spanish vocabulary. FIG. 62B: Learning curves for fine-tuning and evaluating on a new Spanish vocabulary. FIG. 62C: Learning curves for fine-tuning and evaluating on a new English vocabulary. In FIGS. 62B-62C models were either not pre-trained or pre-trained on a different English or Spanish vocabulary. Chance decoding level is 4%. FIG. 62D: Schematic depiction of the paradigm used to evaluate the effect of acoustic similarity between the train and fine-tune set on transfer learning efficacy. FIG. 62E: Mel-cepstral distortion (MCD, as in FIG. 60G) between each word in the acoustically (acou.) similar or different train sets with the corresponding word in the fine-tune/test set (**P<0.005; two-sided Wilocxon Signed-rank test). FIG. 62F: Learning curves for fine-tuning and evaluating on the “Fine-tune and test set”, defined in FIG. 62D with transfer learning from the acoustically (acou.) similar and different models. Learning curves are also shown for no pretraining and transfer learning from a model trained on a semantically (sem.) similar set of words. Performance at 0.0 hours of training data reflects the pretrained model's ability to generalize to the fine-tune and test set with no additional or specific training data. Chance decoding is 10%. In FIGS. 62B-62C and 62F the shaded areas represent 99% confidence intervals.

### 4.4. SUMMARY AND CONCLUSIONS

In this study, shared articulatory representations were leverage in speech motor-cortex to drive a bilingual speech neuroprosthesis. Low word error rates, usable in a clinical setting [59], with high language-classification accuracy were achieve when the target language is freely decoded based on neural features and, importantly, differential linguistic context that builds throughout a phrase. Over 180 weeks after implantation, the ECoG-based neural-classification algorithms demonstrate stable performance without retraining for over 40 days. Robust decoding of syllables in a given language was also observed after training on data collected exclusively in the other language. Correspondingly, transfer learning between languages facilitated learning of a new vocabulary in a second language with as little as one hour of new training data. Together, this set of findings parallels advancements in ASR, bringing communication technology to multilingual and non-English speakers.

This study represents one of the first investigations of bilingual speech production with invasive electrophysiology. The participant learned Spanish natively (L1) and then learned English (L2) later in his adult life, significantly after any critical acquisition period [63]. However, no cortical regions or patterns of neural activity [26,28] specific to English or Spanish speech attempts were found. Despite later acquisition of L2 (after brainstem stroke), no clear differences in magnitude of evoked activity between L1 and L2 (noted in non-invasive neurophysiology studies [29-34]) were noticed. Aligning with theories that the brain fits L2 into the articulatory framework of L1 [39,40], the results demonstrate a largely conserved syllable representation between languages.

This shared articulatory representation offers key advantages for multilingual BCIs. BCIs require training data with the user to develop high-performance decoders. This puts a burden on the user, and long training times may discourage adoption and continued use [64,65]. Here, it is demonstrated that, through transfer learning, training time for multilingual participants to use all their languages may be strongly reduced. Further, the sub-lexical decoders achieved robust performance on syllables in a new language with no additional language-specific training data. These findings suggest that future multilingual speech BCIs may target shared sub-lexical units, for example phonemes, to improve bilingual vocabulary sizes and decoding speeds, leveraging alternative approaches to fixed-window word decoding [8,15,66].

The strength of shared-cortical articulatory representations is encouraging for generalizability to other patients. This is especially true for those who learned L2 early in life, which correlates with stronger shared representations [30,31,33,35]. Future work based on this study may examine whether this effect varies with L2 proficiency, age-of-acquisition, and articulatory similarity to L1. While this study leveraged invasive electrophysiology, the results also hold relevance for non-invasive BCIs. A potential advantage of non-invasive BCIs is the ability to sample a larger distribution of cortex involved in functions beyond speech-motor control. Non-invasive BCIs may apply a similar framework, as applied here with articulatory features, for higher-level language features, such as semantics [67,68], that could further enable rapid generalizability across languages. These studies may also find language-specific signals, which were not seen on speech motor cortex, but may exist in higher-order cortical regions [28,69,70]. A complementary direction for invasive BCIs is to understand whether language-specific signals exist at the level of single-neurons.

While a strong, shared articulatory representation between English and Spanish was demonstrated, it is possible that languages in different families, such as English and a tonal language (e.g. Mandarin), have distinct cortical representations for certain speech-production features, such as pitch [71,72]. These differences could improve neural decoding of the intended language but limit transfer learning between the languages. Additionally, language selection was modeled at the phrase level. In practice, multilingual speakers may switch between languages within phrases (code-switching), a topic that has been studied with non-invasive neural recordings and large language models [73-75]. Future studies based on this work may explore a neural code-switching signal or utilize code-switching language models to decode language on the word level [73]. Overall, this study demonstrates the feasibility of a bilingual speech neuroprosthesis that can flexibly decode intended language and generalize rapidly between languages, having the potential to restore naturalistic communication to the many bilingual speakers who may benefit.

### 4.5. MATERIALS AND MATERIALS

### 4.5.1. Clinical-Trial Overview and the Participant

This study was conducted as part of the BCI Restoration of Arm and Voice (BRAVO) clinical trial (ClinicalTrials.gov; NCT03698149) approved by the FDA, UCSF IRB, and the NIH. All ethical regulations were followed. This trial is aimed at assessing the feasibility of ECoG, and ECoG-based decoding methods, as a long-term assistive neurotechnology to restore naturalistic communication and limb mobility.

The participant (he/him) involved in this study was 36 years old at enrollment. He was previously diagnosed with severe spastic quadriparesis and anarthria by neurologists and a speech-language pathologist due to stroke of the bilateral pons [13]. His injury did not affect cognitive function, and he has slight residual function of the vocal tract allowing for audible grunts and moans; however, he is unable to produce intelligible speech. To communicate, he relies on an Augmentative and Alternative Communication (AAC) interface that utilizes residual head movements to spell out words. After details concerning the neural implant, experimental protocols, and medical risks were explained, the participant provided full informed consent to partake in the study. The participant is a native Spanish speaker (L1) and learned English in adult life reaching fluency at age 30, after his brainstem stroke.

### 4.5.2. Neural Implant

The participant's neural implant was a high-density ECoG array (PMT) coupled with a percutaneous connector (Blackrock Microsystems). 128 electrodes, arranged in a lattice formation, with 4-mm center-to center spacing make up the ECoG array. Over three years ago, the array was surgically implanted on the pial surface of the left hemisphere. The array was centered to sample cortical regions essential for speech production, namely the dorsal posterior aspect of the inferior frontal gyrus, the posterior aspect of the middle frontal gyrus, the precentral gyrus, and the anterior aspect of the postcentral gyrus. In order to transmit data to a computer for further analysis, the percutaneous connector was implanted in the skull. The connector conducts electrical signals from the ECoG array to a detachable digital headstage and cable (NeuroPlex E; Blackrock Microsystems), which transmits the data to a computer with little signal processing.

### 4.5.3. Data Acquisition and Preprocessing

To acquire and extract meaningful neural features for downstream analysis, a multi-step pipeline was applied. First, the local field potential (LFP) was acquired from each electrode via a headstage (a detachable digital connector; NeuroPlex E, Blackrock Microsystems) connected to the percutaneous pedestal connector. The connector digitized the LFP from each electrode and transmitted the signals through an HDMI connection to a digital hub (Blackrock Microsystems). The digital hub relays the digitized signals to a Neuroport system (Blackrock Microsystems) through an optical fiber cable. The Neuroport system applies noise cancellation and an anti-aliasing filter to the signals before streaming them at 1 kHz to a separate online computer via an Ethernet connection (Colfax International). The NeuroPort Central Suite software package (version 7.0.4; Blackrock Microsystems) was used to control the Neuroport system.

The resulting LFP across electrodes is further processed on the online computer, using a custom Python software package (rtNSR) that is capable of processing and analyzing the ECoG signals, executing the online tasks, performing online decoding, and storing the data and task metadata [4,13,42,76]. Common average referencing (CAR) is a useful technique for reducing shared noise across multi-channel neural datasets [77]. rtNSR was used to first apply a CAR (across all electrode channels) to each time sample of the ECoG LFP. Two sets of neural features were then extracted from the re-referenced ECoG signals: high-gamma activity (HGA) and low-frequency signal (LFS). To extract these features, digital finite impulse response (FIR) filters were used to compute the analytic amplitude of the signals in the high-gamma frequency band (70-150 Hz; HGA) and an anti-aliased version of the signals (with a cutoff frequency at 100 Hz; LFS). The time-aligned HGA and LFS were concatenated into a single temporal stream, sampled at 200 Hz. The HGA is then derived from the analytic amplitude whereas LFS is not. Next, the HGA and LFS were z-scored for each channel using a 30-s sliding window approach. Lastly, artifacts in the signal, defined as time points with 32 features with z-score magnitudes greater than 10, were rejected. Each of these time points was replaced with the z-score values from the preceding time point and ignored when updating the 30-s window z-score statistics. These final, processed features defined the HGA and LFS used in subsequent analyses and online decoding (together referred to as “neural features”). All data collection and online decoding tasks were performed in a small office near the participant's residence. To train decoding models and perform offline analyses, data was uploaded to the lab's server infrastructure and models were trained using NVIDIA V100 GPUs hosted on this infrastructure.

### 4.5.4. Task Design: Online Bilingual Neuroprosthesis

To develop a system capable of online decoding of English and Spanish phrases, two general types of tasks were collected with the participant: an isolated-target task and a phrase-decoding task.

Isolated-target task: During the isolated-target task, the text for a target word appeared on the screen along with 4 dots on either side. The dots sequentially disappeared and the text target turned green to indicate the participant should attempt to speak the word (go-cue). The participant attempted to speak the target word at the go cue. A version of the task where the participant was asked not to vocalize (mimed) was first collected. Data included in this paper, is from a subsequent version of the task where the participant was allowed to attempt audible grunts of the target word (overt). During the isolated-target task, a vocabulary of 104 total words was used: 50 Spanish, 51 English, and 3 that are shared across languages (referred to together as “bilingual-words”; Table 34). Neural data collected during the isolated-target task was used to train and optimize classification and detection models that were later used, without recalibration, for phrase-decoding.

Phrase-decoding task: The phrase-decoding task is treated with detail in the Results section and FIG. 59A. In brief, the participant used this paradigm to either perform a copy-typing task with the bilingual-test-phrases (Table 36) or freely use bilingual-words to have a conversation. To choose bilingual-test-phrases and train language models, generation of a large set of phrases made from the bilingual-words was crowdsourced through volunteers independent of the laboratory. To form the bilingual-test-phrases, 28 English and Spanish phrases werc chosen at random that covered the entire vocabulary, had no grammatical errors, and appeared at least 2 times in the generated corpus. Data collected during the phrase-decoding task was used for evaluation only with no calibration of models or hyperparameters based on the bilingual-test-phrases.

### 4.5.5. Task Design: Large-Bilingual-Phrase Set

To further probe the representation of English and Spanish speech attempts, a large-bilingual-phrase set was designed with around 200 unique words in each language (FIG. 61A). These phrases were designed to span a large articulatory space in each language (FIG. 65; Method 4). These phrases were presented to the participant using a sliding cued paradigm. In brief, the participant first heard an audio version of the phrase. The text corresponding to the phrase was then shown on the screen. A green vertical bar then slid across the phrase, at the same rate as the previously presented audio. The green vertical sliding bar indicated the timing of when the participant should attempt each word in the phrase. The neural data collected during this task was used offline to evaluate the representations of English and Spanish speech attempts on speech-motor cortex. No online demonstrations were performed with this task paradigm and phrase set.

### 4.5.6. Task Design: English and Spanish Paired Syllable Words

To probe a shared syllable representation between English and Spanish words, a limited stimuli set was designed where each word shared a syllable, in a slightly different context, with a word in the opposite language (Table 41). This stimuli set was collected using a modified paradigm similar to the isolated-target task. In brief, the participant first heard an audio recording of each word. Next, the text corresponding to the target word appeared on the screen along with 4 dots on either side. The dots sequentially disappeared, and the text target turned green to indicate the participant should attempt to speak the word. The neural data collected during this task was used offline to train and test syllable classifiers between languages. No online demonstrations were performed with this task paradigm and phrase set.

### 4.5.7. Modeling: Online Bilingual Neuroprosthesis

Speech detection and classification models were trained using data collected during the isolated-target task where the participant attempted to speak bilingual-words.

For use with the phrase-decoding task, trained models were saved to the online computer. Models were trained and implemented using the PyTorch Python package (version 1.6.0). Natural language models were also used to encourage the system to decode plausible sequences of words in each language, complementing neural predictions during the phrase-decoding task (Method 2). All hyperparameters for phrase-decoding were chosen and optimized on simulations with the isolated-target task, collected prior to evaluation (Method 1). Common scientific computing packages in Python were used including NumPy, scikit-learn, pandas, seaborn, and matplotlib during modeling and data analysis.

Speech detection: To allow the participant to volitionally engage and disengage the decoding system, a speech detection model was trained to detect attempted speech from neural features online. The speech detector was trained using both low frequency and high-gamma neural features at 200 Hz, using recurrent neural networks (in particular, long short-term memory layers) and truncated backpropagation through time, similar to previously described methods in [13,42]. Model architecture and training parameter details are listed in Table 42.

TABLE 42
Model architecture, training parameters, and detection
parameter values for the speech detection model.

| | | Search- | | Optimal |
|---|---|---|---|---|
| Model | Hyperparameter description | space type | Value range | values |
| Word | Number of GRU layers | by 1 | [1, 4] | 3 |
| classifier | Nodes per GRU layer | by 10 | [200, 300] | 260 |
| | Dropout fraction | by 0.05 | [0.5, 0.9] | 0.8 |
| | Convolution kernel | by 1 | [1, 6] | 4 |
| | size and skip | | | |
| Beam | Language-model scaling factor, α | by 0.1 | [0.1, 1.0] | 0.9 |
| search | Word-insertion weight, β | by 1 | [0, 15] | 11 |
| | Number of beams maintained, B | by 1 | [0, 150] | 150* |

*Probability threshold was slightly adjusted (to the lower value) during the second day of online testing based on qualitative observations.

In this work, the model was trained on overt isolated-target bilingual-words data and rest blocks (where the participant rested silently for 1 minute). For each isolated-target bilingual-words block, each time point was labeled as ‘rest’, ‘speech preparation’, or ‘speech.’ Time points between the presentation of the target word on the screen and the go-cue were labeled as ‘speech preparation’ and time points between the go-cue and 2 seconds after the go-cue were labeled as ‘speech’. This window was chosen based on the duration of average neural responses during speech attempts. Time points from 2 seconds after the go-cue to the end of the trial (when the screen cleared) were discarded from training, due to the ambiguity of when a speech attempt truly ended, and the participant was at rest. All other time points (i.e. the 1 second between trials where the screen was blank) were labeled as ‘rest’. For rest blocks, every time point was labeled as ‘rest’. Rest blocks were added into the training set to augment the amount of time points where the participant was truly resting outside the task context.

The speech detector was trained using all available isolated bilingual-words data prior to online testing. This means that before each day of online testing, the speech detector was retrained if new isolated data was collected the previous day. The speech detector generates the probability of a speech event (causally), and thresholding converts these continuous probability streams into discrete events [13,42]. Detection thresholding parameters (smoothing, probability thresholding, and time thresholding) were manually fine-tuned by evaluating on held-out bilingual phrase blocks that were not used in any part of training the speech detector.

Classification: an artificial neural network (ANN) was trained with the objective of classifying the neural features from a speech attempt as one of the 104 words in the bilingual-words set. During model training, stochastic gradient descent and the Adam optimizer were used to find a set of parameters that minimized the cross entropy loss between target and predicted outputs across the 104 classes. Weights were initialized with parameters learned on the prior collection of mimed bilingual-words, given a small, final improvement on overt with transfer learning from mimed.

In brief, the ANN processed a 3.5 second window of neural features (0-3.5 s relative to the go-cue), corresponding to a speech attempt. 3.5 seconds was chosen for the window length, as it was the speed at which the participant could reliably attempt sequential words using the cued decoding system. The neural features were first decimated by a factor of 6 to 33.33 Hz. A one-dimensional temporal convolutional layer further down sampled the neural features by a factor of 4. The downsampled neural features are then passed through a three layered bidirectional gated recurrent neural network (GRU) [79]. Here, dropout is used to prevent the model from overfitting. Finally, the output of the final timestep of the last layer of the GRU is passed through a dense, fully connected layer to produce 104 outputs, corresponding to each word of the bilingual-words. A softmax function is applied to these outputs to yield an estimated probability of each word. During training, the outputs corresponding to words in a different language than the target word are masked before computation of the loss. This encourages the model to make predictions consistent with the vocabulary of each language. During evaluation, where the language of the speech attempt is unknown, English and Spanish word probabilities were separately passed forward to downstream decoding modules (see Method 1 for specific details regarding data-augmentation and hyperparameter-optimization). A table providing optimal hyperparameters is provided in Table 43.

TABLE 43
Hyperparameter definitions and values
for classification and beam search.

| Parameter type | Parameter description | Value |
|---|---|---|
| Model Architecture | Number of LSTM layers | 3 |
| | Nodes per LSTM layer | 128, 64, 32 |
| | LSTM type | Unidirectional |
| Training parameters | Optimizer | Adam |
| | Loss | Cross-entropy |
| | Learning rate | 0.001 |
| | Batch size | 512 |
| | Dropout | 0.5 |
| Detection parameters | Smoothing size | 78 |
| | Probability threshold | 0.50. 0.49* |
| | Time threshold duration | 100 |

*Higher values were not tested due to increasing computational burden.

During the phrase-decoding task, model ensembling was utilized to minimize overfitting and variance caused by random initialization of parameters [80]. 10 distinct classification models were trained using 10 random subsets of the isolated-target task training set. Each model retained the same architecture but saw a slightly different distribution of training data. To evaluate a given window of neural features, predictions across the 10 models were averaged to yield a single ensemble prediction.

Language modeling: 5-gram natural-language models were trained for both English and Spanish from the crowdsourced corpus, using custom code (Method 2). The language model was trained to output the probability of each word in the vocabulary based on the previous up to 4 words in the sequence.

Phrase decoding: The primary goal of phrase decoding is to find the most likely sequence of words (s*) given neural data X. To solve this problem, a beam search approach with modifications was used to apply to word sequences in two languages. In brief, the neural features for each 3.5 second window were passed through the ensemble classifier to generate a neural-based probability of each word in the vocabulary. These probabilities were then split and rescaled into two vectors: one for Spanish words and another for English words. At this stage, the same downstream decoding modules are applied separately for English and Spanish. The phrase decoding module finds s* given its likelihood from the neural data and its likelihood under the language-model prior. At each window within the trial the highest scoring English beam is compared to the highest scoring Spanish beam. The beam with the higher overall score is displayed to the participant as feedback and effectively sets the language of the output (see Method 3 for a detailed mathematical treatment and information on hyperparameter selection for the beam search process).

### 4.5.8. System Evaluation

Data exclusions: During the phrase-decoding task, the participant was instructed to continue attempting each word in the phrase regardless of decoding accuracy. However, on a small subset of trials, the participant self-reported making an error or not being able to continue the attempt (n=7 out of 168 total trials). Errors included attempting the wrong words in the phrase (n=3, such as attempting in English rather than Spanish), muscle spasms/shakes preventing attempted speech (n=3), and having something in his eye (n=1). These trials were excluded from subsequent analysis to focus on the performance of the system rather than the performance of the participant.

Word error rate: the word error rate (WER) was reported as the sum of the word edit distances between the predicted and target phrases in a phrase-decoding block divided by the total number of words across all target phrases in the block. Each block contained 8 phrases, 4 English and 4 Spanish. It was chosen to report WER over blocks given that short phrases may become overly influential [42,43,76]. To compute the WER in the neural-only condition, verbs were left unconjugated both in the decoded and ground truth sentences and considered up to the point where the ground truth and decoded sentences had the same number of words. Thus, it is the inverse of classification accuracy and lets the neural-classification performance of the system be probed during the sentence-decoding paradigm.

Cross validation accuracies: To evaluate the offline performance of the system in classifying bilingual words, 10-fold cross validation (CV) was used. In each of the 10-folds, 90% of the data is used for training and 10% for evaluation. Within the 90% of data used for training, 10% is randomly selected to be reserved for a validation set (used to early stop training, see Method 2). Within each of the 10 folds, 10 randomly initialized models were fitted to ensemble predictions on the held out evaluation fold. When indicated, during evaluation, the predictions that were not in the same language as the target word were masked. This allowed performance over the English and Spanish vocabularies to be probed separately.

To assess performance over the full 104 bilingual-word vocabulary, models were trained with a single modification. Masking was removed entirely to allow the models to learn associations across languages during training and testing. The CV accuracies for using different window sizes during classification of the full 104 bilingual-word vocabulary were also provide.

Performance without recalibration: To evaluate the performance of the system without daily recalibration, a neural classifier was trained using the same process as described above (Methods: Classification) on data collected before day 1333 post-implantation. The weights of this classifier were then frozen, and its performance was evaluated on isolated-task data collected on subsequent days until the start of online sentence-decoding. Performance with the masking approach was evaluated to compute accuracy over the English and Spanish vocabularies separately or the entire vocabulary. For a comparison, the same methodology was used to retrain the classifier with sequential addition of each day's data.

Electrode contributions to classification: To probe whether English and Spanish words led to similar electrode contributions, classification models were trained only on bilingual-words in English or Spanish. The electrode contributions in these models were then evaluated to compare between the two languages. The contribution of each electrode to classification performance was defined as the derivative of the classifier's loss function with respect to the input features (HGA or LFS) over time [13,42,81]. This effectively measures the change in model outputs given small changes in the HGA or LFS for each electrode across time points. To form a composite contribution per electrode and feature set, the L2-norm was calculated over time and averaged across evaluation trials. For each feature set, the resulting values were then log transformed and normalized so that each value fell between 0-1.

Confusability of English and Spanish words: To probe the confusability between bilingual-words, the predictions from 10-fold CV models trained on all bilingual-words with no masking were used. A confusion matrix was computed where each entry represents the number of times a word (row) was predicted as being any of the 104 words in the vocabulary. Intuitively, the entry on the diagonal then represents the number of times a word was correctly classified. Each row was normalized to sum to 1, making each entry a proportion (0-1). To explore which factors drove confusability between any two words the semantic and acoustic similarity between bilingual-words were measured.

Each word was first embed as a 300 dimensional vector using Word2Vec [52]. The semantic similarity between words was computed as the cosine similarity between respective embedded vectors. An audio waveform was next generated for each word using a multilingual text-to-speech system [82], where a single speaker was used. The mel-cepstral distortion (MCD), commonly used to evaluate speech synthesis systems [53], was used to measure the acoustic similarity between words. The MCD is defined as the squared error between dynamically time warped mel cepstral coefficients of two waveforms.

A multiple regression model was fit to predict the confusability between every pair of bilingual-words based on whether the words were in the same language as well as their semantic and acoustic similarity. To assess the relative variance explained by each factor each variable was removed individually and the drop in R\hat{ }2 compared to the full model was calculated. The three values were then normalized for explained variance to sum to 1. To compute confidence intervals for these estimates the confusion matrix was sampled with replacement 2000 times. For each random sample, the above described procedure was performed to compute the relative variance explained by each factor.

### 4.5.9. a Shared Articulatory Representation Across Languages

To probe a shared articulatory representation, the standard deviation of the average HGA (0-2 seconds after a go-cue) was first computed for speech attempts to English and Spanish phrases within the large-bilingual-phrase set. Evoked response potentials (ERPs) were calculated by averaging the HGA at each timepoint for speech attempts to phrases in each language. The maximum HGA was computed across timepoints of the ERP at each electrode.

The temporal correlations were also tested between the ERPs for English and Spanish phrases. During each iteration of 2000 bootstraps, English and Spanish trials were randomly split into two equal groups. The ERPs for each group were then computed, resulting in 2 English and 2 Spanish ERPs per electrode. At each electrode, the Pearson correlation was computed between the within language and between language ERPs. The correlations across the within and between language groups were averaged to yield two data points per electrode per iteration. The median between language and within language correlation is taken across bootstraps for each electrode.

It was next asked whether temporal patterns in the neural features could be used to classify each trial as an English or Spanish phrase. A classification model was fit, with the same parameters and architecture found optimal for bilingual-words in the isolated-task, to predict whether a trial was an English or Spanish phrase based on a time interval of [−2, 4] s relative to the go cue.

### 4.5.10. Syllable Decoding

To decode syllables from speech attempts in the English and Spanish paired syllable words set, a classifier was trained to predict over the set of shared syllables (Table 41). For syllables that occurred at the beginning of a word, neural features were used from a time window of [0, 2] s relative to the go cue. For syllables that occurred at the end of a word, neural features were used from a time window of [1.5, 3.5] s relative to the go cue. A classification model was then trained, using the same architecture, training procedure (no masking), and hyperparameters (except the dropout was lowered to 0.5 given fewer target classes) as found optimal for bilingual-words in the isolated-target task, to predict probabilities over the shared syllables. To evaluate the ability of classification models to generalize between languages, two evaluation schemes were used. To evaluate performance in the same language used to train the classifier, 10-fold CV was used. To evaluate performance on the other language, not used in training, all syllable pair trials from the non-training language (split into 10 equally sized folds) were used.

### 4.5.11. Transfer Learning Between Languages

To assess the efficacy of transfer learning between languages, data collected during the isolated-target task was used. 25 words were randomly selected from the English bilingual-words and 25 words from the Spanish bilingual-words. Classification models for these vocabularies were then trained using the same architecture, training procedure (no masking), and hyperparameters as found optimal for bilingual-words. This yielded two pretrained models, one in English and another Spanish. Another 25 English words were then randomly selected from the remaining unused English bilingual-words to form a new vocabulary. The above described process was repeated a second time with one change: selecting 25 Spanish words from the remaining unused Spanish bilingual-words to form the new vocabulary.

To compute learning curves with the new vocabulary, 10-fold CV was used. Within each fold, classification models were iteratively trained with increasing fractions of the training data. At each fraction of training data, evaluation was done on the held out evaluation fold. This yields 10 estimates of evaluation accuracy at each fraction of training data included. The 99% confidence interval over these 10 estimates at each fraction of training data studied was visualized.

Learning curves were computed on the new vocabulary, using the above described procedure, under three conditions: no transfer learning (weights initialized randomly), transfer learning from the pre-trained English model, and transfer learning from the pre-trained Spanish model.

### 4.5.12. Statistics

Two-sided non-parametric tests were used to compare groups of observations. For unpaired data, Mann-Whitney U-tests were used and for paired data Wilcoxon signed-rank tests were used. A cutoff of 0.01 was used to determine significance of p-values and a Holm-Bonferroni correction was used to adjust p-values for multiple comparisons where the underlying neural data was not independent. Associated P-values for the Spearman rank correlation were computed with permutation testing. Confidence intervals were computed with a bootstrapping approach. In brief, over 2000 iterations, the data (for example blocks) was randomly sampled with replacement and the desired metric (for example the median word error rate over blocks) was computed. The 99% confidence interval was then taken over the bootstrapped distribution.

### 4.5.13. Videos

Video 1: A demonstration of online word-by-word bilingual sentence decoding from the brain of a participant with paralysis. Decoding is initialized when a speech attempt is detected from cortical activity by the system. The participant next attempts to say each word in the target phrase (top of screen) during consecutive 3.5 second cued windows. The green underline that appears after the countdown indicates the go-cue for each consecutive window. The system deactivates when a speech attempt is not detected during a 3.5 second cued window. During each cued window, a classifier predicts the likelihood of each English and Spanish word in the vocabulary based on the cortical activity. These likelihoods are combined with natural-language models of English and Spanish to output the most likely sentence at each timepoint. The language (English or Spanish) of the most likely sentence is not manually set by the participant; rather, it is flexibly decoded by the classifier and language-models. The system better predicts the intended language as the phrase progresses, highlighting the importance of linguistic context captured by the language models.

Video 2: The participant uses the bilingual speech neuroprosthesis to have an open-ended conversation with a member of the research team. He flexibly switches between using English and Spanish. The system works as in Video 1; however, the participant now produces any sentence he desires, using the system vocabulary. Translations of each sentence are shown for clarity.

### 4.6. SUPPLEMENTARY NOTES

### 4.6.1. Assessment of the Participant's Articulatory Inventory

The participant described in this study (he/him) was diagnosed with anarthria by a certified speech-language pathologist. Anarthria was secondary to brainstem stroke of the bilateral pons. With regard to residual motor-movement, an oral-mechanism test was performed to assess jaw, tongue, and lip function. The participant has severe articulatory deficits, with movement of all three articulators being slow and effortful. The participant was unable to perform lip rounding or maintain a lip closure. Further, tongue movements were severely limited with only a slight ability to slowly extend and raise the tongue. Residual jaw opening and closing was the most reliable but was slow and effortful. To characterize the reliability of syllabic level speech production during vocalized attempts, a perceptual dysarthria assessment was performed. The participant was unable to intelligibly produce sequences of multiple syllables. Breakdowns increasingly occurred as the number or words in the phrase or syllables per word increased. Rates of speech were slow, only 1-2 syllables per breath), and speech was overall characterized by spastic features, leading to articulatory imprecision. Thus, overall, the final diagnosis from speech language pathology was anarthria, defined as a loss of the ability to articulate speech with preserved comprehension.

### 4.6.2. Participant's AAC Speed

The participant's communication speed was quantified using his common Augmentative and Alternative Communication (AAC) strategy. In brief, the participant uses residual head movements to control a laser pointer attached to his glasses to spell out words by directing the laser to letters on a board. The participant was recorded spelling out 30 sentences, 15 English and 15 Spanish, that were randomly chosen from the bilingual-test phrases. The average words per minute was then calculated over these 30 sentences. This is the value displayed on FIG. 59D (3 |97 words per minute). It was also confirmed that the randomly drawn sentences had the same letters/word distribution as the full bilingual-test-phrases (FIG. 66).

### 4.7. Method 1: Articulatory Sampling of the Large-Bilingual-Phrase-Set

To verify that the large-bilingual-phrase-set (FIG. 61A) spanned a range of articulatory features in each language, a few analyses were performed. First, a multilingual transformer based grapheme to phoneme conversion model was used to extract the phonemes across English and Spanish phrases in the large set [83]. Phonemes are defined as the smallest discriminable units of sound that make up a language [84]. For each unique English and Spanish phoneme present in the large-bilingual-phrase-set, the number of occurrences were computed. It was noted that many phonemes in each language are represented. Further, phonemes have place of articulation (POA) features, based on which vocal tract muscle is primarily used to produce the sound [84]. For example the “b” sound is bilabial, being articulated by bringing the lips together. The number of occurrences of each POA feature was computed over the English and Spanish phrases. A similar distribution of POA features between the two languages was noticed, with 5 primary articulatory groups represented. Overall, this provides quantitative evidence that the large-bilingual-phrase-set samples a diverse set of articulatory features in each language.

### 4.8. METHOD 2: LANGUAGE MODELING

### 4.8.1. N-Gram Modeling

During phrase-decoding, two 5-gram language models were used, one in English and another in Spanish, given their ability to guide neural predictions towards linguistically valid phrases. A 5-gram language model emits a probability distribution over words in the vocabulary based on context from the up to 4 previous words. The same training procedure previously described [42] was used. Back-off and discounting was used to improve the contextual estimates of the 5-gram model [85]. Additionally, Kneser-Ney smoothing was applied to improve unigram model estimation. Specifically, a discounting rate of 0.9 was used. 5-gram language models were trained in each language using 700 Spanish and 806 English sentences generated by bilingual volunteers independent from the inventors.

### 4.9. METHOD 3: ISOLATED-TARGET CLASSIFICATION MODEL

### 4.9.1. Data Preparation

Classification models were trained on the isolated-target data for use in downstream phrase-decoding and offline characterizations of English and Spanish speech attempts. For each isolated-target trial, a 3.5 second window of neural features (0-3.5s relative to the go-cue) was used. The neural features were first decimated by a factor of 6 to 33.33 Hz. Next, each time sample was normalized to have an L2-norm of 1 across the HGA and LFS features, separately. These pre-processed neural features were then used to train downstream classifiers.

### 4.9.2. Modeling

Architecture and training: To capture temporal patterns in the neural features, gated-recurrent unit (GRU) layers were primarily used [87]. The pre-processed neural features are first consumed by a 1-dimensional convolutional layer with kernel size and stride of 4. This effectively downsamples the neural features by a factor of 4. The representations from the 1-d convolutional layer are then passed through a stack of 3 GRU layers with 260 units in each layer. Each GRU layer was bidirectional allowing the classifier to learn both forward and backward representations from the neural features. During training, a dropout rate of 0.8 was used minimize overfitting [88]. To compute a probability distribution over the 104 bilingual-words, the representation at the final time point of the final GRU layer was passed through a fully connected layer, with output size 104, yielding activation over the 104 bilingual-words. A softmax function was next applied to rescale the activation to a probability distribution.

Cross entropy loss was used to train the classifier. Mini-batch stochastic gradient descent (batch size 32) was used along with the Adam optimizer [78] to optimize network parameters. Training was stopped after 5 epochs with no improvement to the validation accuracy. The model that had the highest performance on the validation set was kept.

During phrase-decoding, model ensembling was used to improve classification performance by providing robustness to noise and random initialization of weights. 10-fold stratified cross validation was applied on all isolated-target data, collected prior to phrase-decoding, to train 10 unique classification models. During evaluation, the average probability distribution across the 10 models was computed.

Augmentations: To bolster classifier performance, time jittering was used, which has been shown to improve generalization and reduce overfitting for both images [89,90] and neural activity [43,13]. Additionally, a masking procedure was used during training to encourage the classification model to make predictions consistent with the vocabulary of each language. During training, before computation of the cross-entropy loss, predictions that were in a different language than the language of the isolated-target word were masked.

Model pre-training and fine-tuning: A classifier was first trained, using the above-described procedure, on a previously collected isolated-target dataset where the participant silently attempted to speak bilingual-words (rather than producing audible grunts/moans). The weights of this classifier were used to initialize the 10 classifiers trained on the core isolated-target dataset where the participant attempted to speak bilingual-words, expressing some residual, uncoordinated grunts/moans. This process expedited training of the core models used for downstream analysis on the bilingual-words and phrase-decoding.

Hyperparameter optimization: the number of GRU layers, the number of hidden units in each GRU layer, the kernel size and stride of the convolutional layer, the dropout rate, and the learning rate were optimized. Isolated-target trials were used from the two prior recording sessions before the start of the phrase-decoding as a held-out test set. Models were trained on all other isolated-target trials and evaluated on the held-out test set. The ranges provided in (Table 43) were searched for each hyperparameter and the parameters that produced the highest accuracy on the held-out test set were kept. Training was conducted in the same manner as previously described.

### 4.10. METHOD 4: SENTENCE DECODING

### 4.10.1. Task Infrastructure

The phrase-decoding system was designed to decode English and Spanish phrases from cortical activity without the user having to manually select a language. The system continuously acquired neural features, high-gamma activity (HGA; 70-150 Hz) and low-frequency signals (LFS; 0.3-100 Hz), from the local field potential (LFP) of each electrode on the participant's array. The temporal flow of information through the system is as follows (FIG. 63). The participant first makes a speech attempt which is detected by the system, through the speech detection model, and cues activation of an ongoing decoding process. Following the system activating, sequential 3.5s windows are shown to the participant. At the end of each window, after the full 3.5s have passed, the corresponding neural features from that window are passed to the decoding process illustrated in FIG. 59A and FIG. 64. There is then a latency to conduct the beam search and decoding, after which the most likely beam from the process is displayed to the participant monitor. This will continue to occur until a 3.5 window passes where there is no detected speech event. After such a window, the decoding is finalized and terminated. The system then resets and waits for another speech attempt.

### 4.10.2. Verb and Adjective Conjugations

Given that verbs and adjectives, especially in Spanish, may have multiple different conjugations (i.e., traer: traigo, traes, trae, traemos, traen, trayendo), the neural probability for the unconjugated form of the verb was broadcast to all the conjugated forms. This led to an increased vocabulary size of 111 Spanish and 70 English words. The language models were trained on this increased vocabulary size in each language. The beam search is also conducted over these increased vocabulary sizes, and the language model is readily able to infer the correct conjugation based on surrounding context of the verb in the overall phrase.

### 4.10.3. Beam Search

As described previously, a bilingual beam search was used to flexibly decode English and Spanish phrases from cortical neural activity. The goal of this process is to find the most likely sequence of words (s*) given the neural data X. Here, more mathematical detail to the approach is provided. As previously described, an ensembled prediction across the bilingual-words is generated for each 3.5 second window in the trial. These predictions are split and re-scaled across English and Spanish words. Next, a language-specific beam search is applied to find s*, the most likely phrase in each language, based on likelihood under the neural data and language models. This can be expressed mathematically as:

s*=argmaxₛP_{nc}(s|X)*Pₗₘ(s^{α})* |s|^{β}

Here, Pnc(s|X) is the probability of s estimated by the ensembled neural classifier given each 3.5s window of neural activity. This is equivalent to the product of the neural classification probability of each word in s. Plm(sα) is the probability of the phrase s under the language-specific natural language-model down weighted by a factor alpha. The parameter |s|β introduces a word-insertion bonus, β, to counter the decreasing probability under the language model with each word added. The notation |s| indicates the number of words in s. Functionally, since a cued window approach is relied on where the number of words in s* is known based on the number of windows, β has no effect on the decoded phrase; however, it better normalizes scores for number of words, aiding offline analyses.

To approximate s* at each timepoint t=t an iterative beam-search as in [42] was used. A list of the B most likely phrases from t=τ−1 (or an empty string if t=1) is used as a set of candidate prefixes. Here B, denotes the beam width: the number of candidate phrases evaluated. For each candidate prefix 1 and each word in the vocabulary (w), a new phrase was constructed by adding w to 1. These phrases were then re-scored under the formulation provided above. The most likely candidate phrase, s*, was selected at t=t as the new prefix (1+w) with the highest score.

Phrase decoding is run separately for English and Spanish. At each cued-window the highest scoring English beam is compared to the highest scoring Spanish beam. The beam with the higher overall score is displayed to the participant as feedback and effectively sets the language of the output. To choose values for α, β, and B a hyperparameter optimization procedure was used on simulations with data from the isolated-word task. 20 random phrases were selected from the generated bilingual corpus, excluding phrases that appeared in the bilingual-test-phrases. Neural predictions corresponding to isolated-task trials were then used for each word in the phrases to simulate the phrase-decoding procedure. A range of values for α, β, and B (Table 43) were swept and the values that minimized the overall word error rate of the system on the validation phrases were kept.

### 4.11. REFERENCES

The numbering related to the following references apply with respect to the experimental results presented in Example 4:
[1]_Nip, I. & Roth, C. R. Anarthria. in Encyclopedia of Clinical Neuropsychology (eds. Kreutzer, J., DeLuca, J. & Caplan, B.) 1-1 (Springer International Publishing, 2017).
[2]__Chartier, J., Anumanchipalli, G. K., Johnson, K. & Chang, E. F. Encoding of Articulatory Kinematic Trajectories in Human Specch Sensorimotor Cortex. Neuron 98, 1042-1054.c4 (2018).
[3]_Herff, C. et al. Generating Natural, Intelligible Speech From Brain Activity in Motor, Premotor, and Inferior Frontal Cortices. Front. Neurosci. 13, 1267 (2019).
[4]_Moses, D. A., Leonard, M. K., Makin, J. G. & Chang, E. F. Real-time decoding of question-and-answer speech dialogue using human cortical activity. Nat. Commun. 10, 3096 (2019).
[5]_Soroush, P. Z. et al. The nested hierarchy of overt, mouthed, and imagined speech activity evident in intracranial recordings. NeuroImage 269, 119913 (2023).
[6]_Thomas, T. M. et al. Decoding articulatory and phonetic components of naturalistic continuous speech from the distributed language network. J. Neural Eng. 20, 046030 (2023).
[7]_Stavisky, S. D. et al. Neural ensemble dynamics in dorsal motor cortex during speech in people with paralysis. eLife 8, e46015 (2019).
[8]_Willett, F. R. et al. A high-performance speech neuroprosthesis. Nature 1-6 (2023) doi:10.1038/s41586-023-06377-x.
[9]_Wandelt, S. K. et al. Decoding grasp and speech signals from the cortical grasp circuit in a tetraplegic human. Neuron 110, 1777-1787.e3 (2022).
[10]_Angrick, M. et al. Speech synthesis from ECoG using densely connected 3D convolutional neural networks. J. Neural Eng. 16, 036019 (2019).
[11]_Berezutskaya, J. et al. Direct Speech Reconstruction from Sensorimotor Brain Activity with Optimized Deep Learning Models. http://biorxiv.org/lookup/doi/10.1101/2022.08.02.502503 (2022) doi:10.1101/2022.08.02.502503.
[12]_Dash, D., Ferrari, P. & Wang, J. Decoding Imagined and Spoken Phrases From Non-invasive Neural (MEG) Signals. Front. Neurosci. 14, 290 (2020).
[13]_Moses, D. A. et al. Neuroprosthesis for Decoding Speech in a Paralyzed Person with Anarthria. N. Engl. J. Med. 385, 217-227 (2021).
[14]_Mugler, E. M. et al. Direct classification of all American English phonemes using signals from functional speech motor cortex. J. Neural Eng. 11, 035015-035015 (2014).
[15]_Metzger, S. L. et al. A high-performance neuroprosthesis for speech decoding and avatar control. Nature (2023) doi:10.1038/s41586-023-06443-4.
[16]_Choe, J. et al. Language-specific Effects on Automatic Speech Recognition Errors for World Englishes. in Proceedings of the 29th International Conference on Computational Linguistics 7177-7186 (International Committee on Computational Linguistics, 2022).
[17]_DiChristofano, A., Shuster, H., Chandra, S. & Patwari, N. Global Performance Disparities Between English-Language Accents in Automatic Speech Recognition. Preprint at http://arxiv.org/abs/2208.01157 (2023).
[18]_Baker, C. & Jones, S. Encyclopedia of Bilingualism and Bilingual Education. (1998).
[19]_Athanasopoulos, P. et al. Two languages, two minds: flexible cognitive processing driven by language of operation. Psychol. Sci. 26, 518-526 (2015).
[20]_Chen, S. X. & Bond, M. H. Two languages, two personalities? Examining language effects on the expression of personality in a bilingual context. Pers. Soc. Psychol. Bull. 36, 1514-1528 (2010).
[21._Costa, A. & Sebastián-Galles, N. How does the bilingual experience sculpt the brain? Nat. Rev. Neurosci. 15, 336-345 (2014).
[22]_Naranowicz, M., Jankowiak, K. & Behnke, M. Native and non-native language contexts differently modulate mood-driven electrodermal activity. Sci. Rep. 12, 22361 (2022).
[23]_Li, Q. et al. Monolingual and bilingual language networks in healthy subjects using functional MRI and graph theory. Sci. Rep. 11, 10568 (2021).
[24]_Pierce, L. J., Chen, J.-K., Delcenserie, A., Genesee, F. & Klein, D. Past experience shapes ongoing neural patterns for language. Nat. Commun. 6, 10073 (2015).
[25]_Dehaene, S. Fitting two languages into one brain. Brain 122, 2207-2208 (1999).
[26]_Kim, K. H. S., Relkin, N. R., Lee, K.-M. & Hirsch, J. Distinct cortical areas associated with native and second languages. Nature 388, 171-174 (1997).
[27]_Tham, W. W. P. et al. Phonological processing in Chinese-English bilingual biscriptals: An fMRI study. NeuroImage 28, 579-587 (2005).
[28]_Xu, M., Baldauf, D., Chang, C. Q., Desimone, R. & Tan, L. H. Distinct distributed patterns of neural activity are associated with two languages in the bilingual brain. Sci. Adv. 3, e1603309 (2017).
[29]_Berken, J. A. et al. Neural activation in speech production and reading aloud in native and non-native languages. NeuroImage 112, 208-217 (2015).
[30]_Del Maschio, N. & Abutalebi, J. The Handbook of the Neuroscience of Multilingualism. (2019).
[31]_DeLuca, V., Rothman, J., Bialystok, E. & Pliatsikas, C. Redefining bilingualism as a spectrum of experiences that differentially affects brain structure and function. Proc. Natl. Acad. Sci. 116, 7565-7574 (2019).
[32]_Liu, H., Hu, Z., Guo, T. & Peng, D. Speaking words in two languages with one brain: neural overlap and dissociation. Brain Res. 1316, 75-82 (2010).
[33]_Shimada, K. et al. Fluency-dependent cortical activation associated with speech production and comprehension in second language learners. Neuroscience 300, 474-492 (2015).
[34]_Treutler, M. & Soros, P. Functional MRI of Native and Non-native Speech Sound Production in Sequential German-English Bilinguals. Front. Hum. Neurosci. 15, 683277 (2021).
[35]_Cao, F., Tao, R., Liu, L., Perfetti, C. A. & Booth, J. R. High Proficiency in a Second Language is Characterized by Greater Involvement of the First Language Network: Evidence from Chinese Learners of English. J. Cogn. Neurosci. 25, 1649-1663 (2013).
[36]_Geng, S. et al. Intersecting distributed networks support convergent linguistic functioning across different languages in bilinguals. Commun. Biol. 6, 1-13 (2023).
[37]_Malik-Moraleda, S. et al. An investigation across 45 languages and 12 language families reveals a universal language network. Nat. Neurosci. 25, 1014-1019 (2022).
[38]_Perani, D. & Abutalebi, J. The neural basis of first and second language processing. Curr. Opin. Neurobiol. 15, 202-206 (2005).
[39]_Alario, F.-X., Goslin, J., Michel, V. & Laganaro, M. The Functional Origin of the Foreign Accent: Evidence From the Syllable-Frequency Effect in Bilingual Speakers. Psychol. Sci. 21, 15-20 (2010).
[40]_Simmonds, A., Wise, R. & Leech, R. Two Tongues, One Brain: Imaging Bilingual Speech Production. Front. Psychol. 2, (2011).
[41]_Hannun, A. Y., Maas, A. L., Jurafsky, D. & Ng, A. Y. First-Pass Large Vocabulary Continuous Speech Recognition using Bi-Directional Recurrent DNNs. ArXiv14082873 Cs (2014).
[42]_Metzger, S. L. et al. Generalizable spelling using a speech neuroprosthesis in an individual with severe limb and vocal paralysis. Nat. Commun. 13, 6510 (2022).
[43]_Willett, F. R., Avansino, D. T., Hochberg, L. R., Henderson, J. M. & Shenoy, K. V. High-performance brain-to-text communication via handwriting. Nature 593, 249-254 (2021).
[44]_Radford, A. et al. Language Models are Unsupervised Multitask Learners. 24 (2018).
[45]_Blakely, T., Miller, K. J., Zanos, S. P., Rao, R. P. N. & Ojemann, J. G. Robust, long-term control of an electrocorticographic brain-computer interface with fixed parameters. Neurosurg. Focus 27, E13 (2009).
[46]_Pels, E. G. M. et al. Stability of a chronic implanted brain-computer interface in late-stage amyotrophic lateral sclerosis. Clin. Neurophysiol. 130, 1798-1803 (2019).
[47]_Silversmith, D. B. et al. Plug-and-play control of a brain-computer interface through neural map stabilization. Nat. Biotechnol. 39, 326-335 (2021).
[48]_Volkova, K., Lebedev, M. A., Kaplan, A. & Ossadtchi, A. Decoding Movement From Electrocorticographic Activity: A Review. Front. Neuroinformatics 13, (2019).
[49]_Luo, S. et al. Stable Decoding from a Speech BCI Enables Control for an Individual with ALS without Recalibration for 3 Months. Adv. Sci. Weinh. Baden-Wurtt. Ger. e2304853 (2023) doi:10.1002/advs.202304853.
[50]_Bouchard, K. E., Mesgarani, N., Johnson, K. & Chang, E. F. Functional organization of human sensorimotor cortex for speech articulation. Nature 495, 327-332 (2013).
[51]_Carey, D., Krishnan, S., Callaghan, M. F., Sereno, M. I. & Dick, F. Functional and Quantitative MRI Mapping of Somatomotor Representations of Human Supralaryngeal Vocal Tract. Cereb. Cortex 27, 265-278 (2017).
[52]_Mikolov, T., Chen, K., Corrado, G. & Dean, J. Efficient Estimation of Word Representations in Vector Space. arXiv.org https://arxiv.org/abs/1301.3781v3 (2013).
[53]_Kubichek, R. Mel-cepstral distance measure for objective speech quality assessment. in Proceedings of IEEE Pacific Rim Conference on Communications Computers and Signal Processing vol. 1 125-128 (IEEE, 1993).
[54]_Mitra, V. et al. Joint modeling of articulatory and acoustic spaces for continuous speech recognition tasks. in 2017 IEEE International Conference on Acoustics, Speech and Signal Processing (ICASSP) 5205-5209 (IEEE, 2017). doi:10.1109/ICASSP.2017.7953149.
[55]_Caruana, R. Multitask Learning. Mach. Learn. 28, 41-75 (1997).
[56]_Tan, C. et al. A Survey on Deep Transfer Learning. in Artificial Neural Networks and Machine Learning-ICANN 2018 (eds. Kůrková, V., Manolopoulos, Y., Hammer, B., Iliadis, L. & Maglogiannis, I.) 270-279 (Springer International Publishing, 2018). doi:10.1007/978-3-030-01424-7_27.
[57]_Makin, J. G., Moses, D. A. & Chang, E. F. Machine translation of cortical activity to text with an encoder-decoder framework. Nat. Neurosci. 23, 575-582 (2020).
[58]_Peterson, S. M., Steine-Hanson, Z., Davis, N., Rao, R. P. N. & Brunton, B. W. Generalized neural decoders for transfer learning across participants and recording modalities. J. Neural Eng. 18, 026014 (2021).
[59]_Watanabe, S., Delcroix, M., Metze, F. & Hershey, J. R. New era for robust speech recognition: exploiting deep learning. (Springer-Verlag, 2017).
[60]_Gao, H. et al. Domain Generalization for Language-Independent Automatic Speech Recognition. Front. Artif. Intell. 5, 806274 (2022).
[61]_Radford, A. et al. Robust Speech Recognition via Large-Scale Weak Supervision. Preprint at http://arxiv.org/abs/2212.04356 (2022).
[62]_Zhang, Y. et al. Google USM: Scaling Automatic Speech Recognition Beyond 100 Languages. Preprint at http://arxiv.org/abs/2303.01037 (2023).
[63]_Hartshorne, J. K., Tenenbaum, J. B. & Pinker, S. A critical period for second language acquisition: Evidence from 2/3 million English speakers. Cognition 177, 263-277 (2018).
[64]_Huggins, J. E., Wren, P. A. & Gruis, K. L. What would brain-computer interface users want? Opinions and priorities of potential users with amyotrophic lateral sclerosis. Amyotroph. Lateral Scler. 12, 318-324 (2011).
[65]_Peters, B. et al. Brain-Computer Interface Users Speak Up: The Virtual Users' Forum at the 2013 International Brain-Computer Interface Meeting. Arch. Phys. Med. Rehabil. 96, S33-S37 (2015).
[66]_Herff, C. et al. Brain-to-text: decoding spoken phrases from phone representations in the brain. Front. Neurosci. 9, (2015).
[67]_Tang, J., LeBel, A., Jain, S. & Huth, A. G. Semantic reconstruction of continuous language from non-invasive brain recordings. Nat. Neurosci. 26, 858-866 (2023).
[68]_Correia, J. et al. Brain-Based Translation: fMRI Decoding of Spoken Words in Bilinguals Reveals Language-Independent Semantic Representations in Anterior Temporal Lobe. J. Neurosci. 34, 332-338 (2014).
[69]_Lucas, T. H., McKhann, G. M. & Ojemann, G. A. Functional separation of languages in the bilingual brain: a comparison of electrical stimulation language mapping in 25 bilingual patients and 117 monolingual control patients. J. Neurosurg. 101, 449-457 (2004).
[70]_Giussani, C., Roux, F.-E., Lubrano, V., Gaini, S. M. & Bello, L. Review of language organisation in bilingual patients: what can we learn from direct brain mapping? Acta Neurochir. (Wien) 149, 1109-1116; discussion 1116 (2007).
[71]_Best, C. T. The Diversity of Tone Languages and the Roles of Pitch Variation in Non-tone Languages: Considerations for Tone Perception Research. Front. Psychol. 10, (2019).
[72]_Li, Y., Tang, C., Lu, J., Wu, J. & Chang, E. F. Human cortical encoding of pitch in tonal and non-tonal languages. Nat. Commun. 12, 1161 (2021).
[73]_Lee, G. & Li, H. Modeling Code-Switch Languages Using Bilingual Parallel Corpus. in Proceedings of the 58th Annual Meeting of the Association for Computational Linguistics 860-870 (Association for Computational Linguistics, 2020). doi:10.18653/v1/2020.acl-main.80.
[74]_Rossi, E., Dussias, P. E., Diaz, M., van Hell, J. G. & Newman, S. Neural signatures of inhibitory control in intra-sentential code-switching: Evidence from fMRI. J. Neurolinguistics 57, 100938 (2021).
[75]_Zheng, X., Roelofs, A., Erkan, H. & Lemhöfer, K. Dynamics of inhibitory control during bilingual speech production: An electrophysiological study. Neuropsychologia 140, 107387 (2020).
[76]_Moses, D. A., Leonard, M. K. & Chang, E. F. Real-time classification of auditory sentences using evoked cortical activity in humans. J. Neural Eng. 15, (2018).
[77]_Ludwig, K. A. et al. Using a common average reference to improve cortical neuron recordings from microelectrode arrays. J. Neurophysiol. 101, 1679-89 (2009).
[78]_Kingma, D. P. & Ba, J. Adam: A Method for Stochastic Optimization. ArXiv14126980 Cs (2017).
[79]_Cho, K. et al. Learning Phrase Representations using RNN Encoder-Decoder for Statistical Machine Translation. ArXiv14061078 Cs Stat (2014).
[80]_Fort, S., Hu, H. & Lakshminarayanan, B. Deep Ensembles: A Loss Landscape Perspective. ArXiv191202757 Cs Stat (2020).
[81]_Simonyan, K., Vedaldi, A. & Zisserman, A. Deep Inside Convolutional Networks: Visualising Image Classification Models and Saliency Maps. ArXiv13126034 Cs (2014).
[82]_Lux, F., Koch, J., Schweitzer, A. & Vu, N. T. The IMS Toucan system for the Blizzard Challenge 2021. (2021).
[83] Zhu J, Zhang C, and Jurgens D. ByT5 model for massively multilingual grapheme-to-phoneme conversion. arXiv preprint arXiv: 2204.03067 2022.
[84] Ladefoged P and Johnson K. A course in phonetics. Cengage learning, 2014.
[85] Jurafsky D and Martin JH. Speech and language processing. Vol. 3. US: Prentice Hall 2014.
[86] Kneser R and Ney H. Improved backing-off for m-gram language modeling. In: 1995 international conference on acoustics, speech, and signal processing. Vol. 1. IEEE. 1995:181-4.
[87] Cho K, Van Merri “enboer B, Bahdanau D, and Bengio Y. On the properties of neural machine translation: Encoder-decoder approaches. arXiv preprint arXiv: 1409.1259 2014.
[88] Hinton G E, Srivastava N, Krizhevsky A, Sutskever I, and Salakhutdinov RR. Improving neural networks by preventing co-adaptation of feature detectors. arXiv preprint arXiv:1207.0580 2012.
[89] Krizhevsky A, Sutskever I, and Hinton GE. Imagenet classification with deep convolutional neural networks. Advances in neural information processing systems 2012; 25:1097-105.
[90] Reed C J, Metzger S, Srinivas A, Darrell T, and Keutzer K. Selfaugment: Automatic augmentation policies for self-supervised learning. In: Proceedings of the IEEE/CVF Conference on Computer Vision and Pattern Recognition. 2021:2674-83.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of assisting a subject with communication, the method comprising:
positioning a neural recording device comprising an electrode at a location in a sensorimotor cortex region of the brain of the subject to record brain electrical signal data associated with attempted speech by the subject;
positioning an interface in communication with a computing device at a location on the head of the subject, wherein the interface is connected to the neural recording device;
recording the brain electrical signal data associated with attempted speech by the subject using the neural recording device, wherein the interface receives the brain electrical signal data from the neural recording device and transmits the brain electrical signal data to a processor of the computing device; and
decoding one or more speech sounds from the recorded brain electrical signal data using the processor, wherein the processor is programmed to use a machine learning model for the decoding.

2. The method of claim 1, wherein the subject has difficulty with said communication because of anarthria, a stroke, a traumatic brain injury, a brain tumor, or amyotrophic lateral sclerosis.

3. The method of claim 1 or 2, wherein the subject is paralyzed.

4. The method of any of claims 1-3, wherein the subject has a speech intelligibility of 10% or less for prompted words.

5. The method of any of claims 1-4, wherein the location of the neural recording device is on a surface of the sensorimotor cortex region or within the sensorimotor cortex region.

6. The method of any of claims 1-5, wherein the electrode of the neural recording device is positioned on the pial surface of the sensorimotor cortex.

7. The method of any of claims 1-6, wherein the neural recording device is positioned such that the recording device covers regions associated with speech production and language perception.

8. The method of claim 7, wherein the covered regions include a middle portion of the superior and/or middle temporal gyrus, the precentral gyrus, and/or the postcentral gyrus.

9. The method of any of claims 1-8, wherein the neural recording device is centered on the central sulcus.

10. The method of any of claims 1-9, wherein the neural recording device comprises an electrocorticography (ECoG) electrode array.

11. The method of claim 10, wherein the ECoG electrode array is a high-density array.

12. The method of claim 11, wherein the high-density array comprises 200 electrodes or more.

13. The method of any of claims 10-12, wherein the electrode array comprises non-penetrating surface electrodes.

14. The method of any of claims 1-13, wherein the interface comprises a percutaneous pedestal connector attached to the subject's cranium.

15. The method of any of claims 1-14, wherein the interface further comprises a headstage connected to the percutaneous pedestal connector.

16. The method of claim 15, wherein the headstage processes and digitizes the brain electrical signal data before transmitting the data to the processor.

17. The method of any of claims 1-16, wherein the electrical signal data comprises high-gamma frequency content features.

18. The method of any of claims 1-17, wherein the electrical signal data comprises neural oscillations in a range from 70 Hz to 150 Hz.

19. The method of any of claims 1-18, wherein the electrical signal data comprises low-frequency signals.

20. The method of claim 19, wherein the electrical signal data comprises neural oscillations in a range from 0.3 Hz to 17 Hz.

21. The method of any of claims 1-20, wherein the brain electrical signal data is recorded from a sensorimotor cortex region selected from the precentral gyrus, postcentral gyrus, superior temporal gyrus, middle temporal gyrus, or any combination thereof.

22. The method of any of claims 1-21, wherein the method further comprises mapping the brain of the subject to identify an optimal location for positioning the electrode for recording the brain electrical signals associated with the attempted speech by the subject.

23. The method of any of claims 1-22, wherein the one or more speech sounds form a word.

24. The method of claim 23, wherein the one or more speech sounds form a sentence.

25. The method of claim 23 or 24, wherein the subject is limited to a specified word set for the attempted speech.

26. The method of claim 25, wherein the word set comprises words for expressing basic concepts and/or caregiving needs.

27. The method of claim 25 or 26, wherein the word set comprises 100 words or more.

28. The method of claim 27, wherein the word set comprises 350 words or more.

29. The method of claim 28, wherein the word set comprises 1000 words or more.

30. The method of any of claims 25-29, wherein the subject is limited to two or more word sets for the attempted speech.

31. The method of claim 30, wherein the subject may switch between word sets.

32. The method of any of claims 1-31, wherein the decoding machine learning model comprises a neural network.

33. The method of claim 32, wherein the neural network comprises one or more convolutional layers.

34. The method of claim 32 or 33, wherein the neural network is bidirectional.

35. The method of claim 34, wherein the neural network is a recurrent neural network (RNN).

36. The method of claim 35, wherein the RNN comprises gated recurrent units (GRUs) or long-short term memory (LSTM).

37. The method of claim 36, wherein the RNN comprises GRUs.

38. The method of any of claims 1-37, wherein one or more discrete speech units of a set of discrete speech units are decoded from the recorded brain electrical signal data.

39. The method of claim 38, wherein each speech sound is decoded from one or more discrete speech units.

40. The method of claim 38 or 39, wherein the set of discrete speech units comprises 50 or more discrete speech units.

41. The method of claim 40, wherein the set of discrete speech units comprises 100 or more discrete speech units.

42. The method of any of claims 36-39, wherein discrete speech units are continuously decoded at a uniform frequency.

43. The method of claim 42, wherein the frequency is 50 Hz or more.

44. The method of claim 43, wherein the frequency is 200 Hz or more.

45. The method of any of claims 38-44, wherein the set of discrete speech units are generated using an encoding machine learning model.

46. The method of claim 45, wherein the encoding machine learning model comprises a neural network.

47. The method of claim 46, wherein the neural network comprises one or more convolutional layers.

48. The method of claim 46 or 47, wherein the neural network is bidirectional.

49. The method of claim 48, wherein the neural network comprises a transformer encoder.

50. The method of claim 49, wherein the machine learning model is a Hidden-Unit Bidirectional Encoder Representations from Transformers (HuBERT) model.

51. The method of any of claims 45-50, wherein the set of discrete speech units are generated by training the encoding machine learning model.

52. The method of claim 51, wherein the training is self-supervised training.

53. The method of any of claims 45-52, wherein the method further comprises:
obtaining reference electronic speech waveforms for a plurality of phrases;
encoding each reference speech waveform into a temporal sequence of discrete speech units using the encoding model;
recording the brain electrical signal data associated with attempted speech by the subject for each one of the plurality of phrases;
training the decoding machine learning model to predict the most likely discrete speech unit associated with a segment of electrical signal data using the speech units derived from the reference speech waveforms and the brain electrical signal data associated with attempted speech for each one of the plurality of phrases.

54. The method of claim 53, wherein the reference speech waveforms are obtained from a recruited speaker.

55. The method of claim 53, wherein the reference speech waveforms are obtained using a text-to-speech algorithm.

56. The method of any of claims 53-55, wherein the decoding machine learning model is trained to learn mappings between neural activity patterns of electrical signals in the brain electrical signal data and the discrete speech units.

57. The method of any of claims 53-56, wherein the training uses a CTC loss function.

58. The method of any of claims 39-57, wherein each speech sound is decoded from one or more discrete speech units using a speech synthesizer.

59. The method of claim 58, wherein the speech synthesizer comprises a machine learning model.

60. The method of claim 59, wherein the synthesizing machine learning model comprises a neural network.

61. The method of claim 60, wherein the neural network comprises one or more convolutional layers.

62. The method of claim 60 or 61, wherein the neural network is bidirectional.

63. The method of claim 62, wherein the neural network is an RNN.

64. The method of claim 63, wherein the RNN comprises GRUs or LSTM.

65. The method of claim 64, wherein the RNN comprises one or more LSTM layers.

66. The method of any of claims 60-65, wherein the neural network comprises an attention mechanism.

67. The method of any of claims 59-66, wherein the synthesizing machine learning model is configured to generate a spectrogram from the one or more discrete speech units.

68. The method of claim 67, wherein the spectrogram is a mel spectrogram.

69. The method of claim 67 or 68, wherein the speech synthesizer further comprises a vocoder configured to synthesize an electronic speech waveform from the spectrogram.

70. The method of claim 69, wherein the vocoder comprises a machine learning model.

71. The method of claim 70, wherein the vocoder machine learning model comprises a neural network.

72. The method of claim 71, wherein the neural network is an RNN.

73. The method of any of claims 69-72, wherein the method further comprises transforming the electronic speech waveform into a personalized electronic speech waveform.

74. The method of claim 73, wherein the personalized electronic speech waveform resembles speech in the subject's own voice.

75. The method of claim 73 or 74, wherein the transforming is performed using a machine learning model.

76. The method of claim 75, wherein the transforming machine learning model comprises a neural network.

77. The method of claim 76, wherein the neural network is based on transformer architecture.

78. The method of any of claims 69-77, wherein the method further comprises converting the electronic speech waveform into an audible speech waveform.

79. The method of claim 78, wherein the electronic speech waveform is converted into an audible speech waveform using a loudspeaker.

80. The method of any of the preceding claims, wherein the processor is provided by a computer or handheld device.

81. The method of claim 80, wherein the handheld device is a cell phone or a tablet.

82. A non-transitory computer-readable medium comprising program instructions that, when executed by a processor, cause the processor to perform the method of any one of claims 1-81.

83. A kit comprising the non-transitory computer-readable medium of claim 82 and instructions for decoding brain electrical signal data associated with attempted speech by a subject.

84. A system for decoding speech sounds from recorded brain electrical signal data configured to perform the method according to any of claims 1-81.

85. A computer implemented method for decoding speech audio from recorded brain electrical signal data associated with attempted speech by a subject, the computer performing steps comprising:
receiving the recorded brain electrical signal data associated with the attempted speech by the subject; and
decoding one or more speech sounds from the recorded brain electrical signal data using a machine learning model.

86. The computer implemented method of claim 85, wherein the decoding machine learning model comprises a neural network.

87. The computer implemented method of claim 86, wherein the neural network comprises one or more convolutional layers.

88. The computer implemented method of claim 86 or 87, wherein the neural network is bidirectional.

89. The computer implemented method of claim 88, wherein the neural network is a recurrent neural network (RNN).

90. The computer implemented method of claim 89, wherein the RNN comprises gated recurrent units (GRUs) or long-short term memory (LSTM).

91. The computer implemented method of claim 90, wherein the RNN comprises GRUs.

92. The computer implemented method of any of claims 85-91, wherein one or more discrete speech units of a set of discrete speech units are decoded from the recorded brain electrical signal data.

93. The computer implemented method of claim 92, wherein each speech sound is decoded from one or more discrete speech units.

94. The computer implemented method of claim 92 or 93, wherein the set of discrete speech units comprises 50 or more discrete speech units.

95. The computer implemented method of claim 94, wherein the set of discrete speech units comprises 100 or more discrete speech units.

96. The computer implemented method of any of claims 92-95, wherein discrete speech units are continuously decoded at a uniform frequency.

97. The computer implemented method of claim 96, wherein the frequency is 50 Hz or more.

98. The computer implemented method of claim 97, wherein the frequency is 200 Hz or more.

99. The computer implemented method of any of claims 92-98, wherein the set of discrete speech units are generated using an encoding machine learning model.

100. The computer implemented method of claim 99, wherein the encoding machine learning model comprises a neural network.

101. The computer implemented method of claim 100, wherein the neural network comprises one or more convolutional layers.

102. The computer implemented method of claim 100 or 101, wherein the neural network is bidirectional.

103. The computer implemented method of claim 102, wherein the neural network comprises a transformer encoder.

104. The computer implemented method of claim 103, wherein the machine learning model is a Hidden-Unit Bidirectional Encoder Representations from Transformers (HuBERT) model.

105. The computer implemented method of any of claims 99-104, wherein the set of discrete speech units are generated by training the encoding machine learning model.

106. The computer implemented method of claim 105, wherein the training is self-supervised training.

107. The computer implemented method of any of claims 99-106, wherein the method further comprises:
receiving reference electronic speech waveforms for a plurality of phrases;
encoding each reference speech waveform into a temporal sequence of discrete speech units using the encoding model;
receiving brain electrical signal data associated with attempted speech by the subject for each one of the plurality of phrases;
training the decoding machine learning model to predict the most likely discrete speech unit associated with a segment of electrical signal data using the speech units derived from the reference speech waveforms and the brain electrical signal data associated with attempted speech for each one of the plurality of phrases.

108. The computer implemented method of claim 107, wherein the reference speech waveforms are generated using a text-to-speech algorithm.

109. The computer implemented method of claim 107 or 108, wherein the decoding machine learning model is trained to learn mappings between neural activity patterns of electrical signals in the brain electrical signal data and the discrete speech units.

110. The computer implemented method of any of claims 93-109, wherein each speech sound is decoded from one or more discrete speech units using a speech synthesizer.

111. The computer implemented method of claim 110, wherein the speech synthesizer comprises a machine learning model.

112. The computer implemented method of claim 111, wherein the synthesizing machine learning model comprises a neural network.

113. The computer implemented method of claim 112, wherein the neural network comprises one or more convolutional layers.

114. The computer implemented method of claim 112 or 113, wherein the neural network is bidirectional.

115. The computer implemented method of claim 114, wherein the neural network is an RNN.

116. The computer implemented method of claim 115, wherein the RNN comprises GRUs or LSTM.

117. The computer implemented method of claim 116, wherein the RNN comprises one or more LSTM layers.

118. The computer implemented method of any of claims 112-117, wherein the neural network comprises an attention mechanism.

119. The computer implemented method of any of claims 111-118, wherein the synthesizing machine learning model is configured to generate a spectrogram from the one or more discrete speech units.

120. The computer implemented method of claim 119, wherein the spectrogram is a mel spectrogram.

121. The computer implemented method of claim 119 or 120, wherein the speech synthesizer further comprises a vocoder configured to synthesize an electronic speech waveform from the spectrogram.

122. The computer implemented method of claim 121, wherein the vocoder comprises a machine learning model.

123. The computer implemented method of claim 122, wherein the vocoder machine learning model comprises a neural network.

124. The computer implemented method of claim 123, wherein the neural network is an RNN.

125. The computer implemented method of any of claims 121-124, wherein the method further comprises transforming the electronic speech waveform into a personalized electronic speech waveform.

126. The computer implemented method of claim 125, wherein the personalized electronic speech waveform resembles speech in the subject's own voice.

127. The computer implemented method of claim 125 or 126, wherein the transforming is performed using a machine learning model.

128. The computer implemented method of claim 127, wherein the transforming machine learning model comprises a neural network.

129. The computer implemented method of claim 128, wherein the neural network is based on transformer architecture.

131. A non-transitory computer-readable medium comprising program instructions that, when executed by a processor, cause the processor to perform the computer implemented method of any one of claims 85-129.

132. A kit comprising the non-transitory computer-readable medium of claim 131 and instructions for decoding brain electrical signal data associated with attempted speech by a subject.

133. A system for producing speech audio directly from neural activity, the system comprising:
a neural recording device comprising an electrode adapted for positioning at a location in a sensorimotor cortex region of the brain of the subject to record brain electrical signal data associated with attempted speech by the subject;
a processor programmed to use a machine learning model to decode one or more speech sounds from the recorded brain electrical signal data;
an interface in communication with a computing device, said interface adapted for positioning at a location on the head of the subject, wherein the interface receives the brain electrical signal data from the neural recording device and transmits the brain electrical signal data to the processor; and
an audio speaker for playing the one or more speech sounds from the recorded brain electrical signal data.

134. The system of claim 133, wherein the subject has difficulty with said speech because of anarthria, a stroke, a traumatic brain injury, a brain tumor, or amyotrophic lateral sclerosis.

135. The system of any of claims 133-134, wherein the neural recording device is adapted for positioning on a surface of the sensorimotor cortex region or within the sensorimotor cortex region.

136. The system of any of claims 133-135, wherein the electrode of the neural recording device is adapted for positioning on the pial surface of the sensorimotor cortex.

137. The system of any of claims 133-136, wherein the neural recording device is adapted to be positioned such that the recording device covers regions associated with speech production and language perception.

138. The system of claim 137, wherein the covered regions include a middle portion of the superior and/or middle temporal gyrus, the precentral gyrus, and/or the postcentral gyrus.

139. The system of any of claims 133-138, wherein the neural recording device is adapted to be centered on the central sulcus.

140. The system of any of claims 133-139, wherein the neural recording device comprises an electrocorticography (ECoG) electrode array.

141. The system of claim 140, wherein the ECoG electrode array is a high-density array.

142. The system of claim 141, wherein the high-density array comprises 200 electrodes or more.

143. The system of any of claims 140-142, wherein the electrode array comprises non-penetrating surface electrodes.

144. The system of any of claims 133-143, wherein the interface comprises a percutaneous pedestal connector attached to the subject's cranium.

145. The system of any of claims 133-144, wherein the interface further comprises a headstage connected to the percutaneous pedestal connector.

146. The system of claim 145, wherein the headstage is configured to process and digitize the brain electrical signal data before transmitting the data to the processor.

147. The system of any of claims 133-146, wherein the electrical signal data comprises high-gamma frequency content features.

148. The system of any of claims 133-147, wherein the electrical signal data comprises neural oscillations in a range from 70 Hz to 150 Hz.

149. The system of any of claims 133-148, wherein the electrical signal data comprises low-frequency signals.

150. The system of claim 149, wherein the electrical signal data comprises neural oscillations in a range from 0.3 Hz to 17 Hz.

151. The system of any of claims 133-150, wherein the brain electrical signal data is recorded from a sensorimotor cortex region selected from the precentral gyrus, postcentral gyrus, superior temporal gyrus, middle temporal gyrus, or any combination thereof.

153. The system of any of claims 133-152, wherein the one or more speech sounds form a word.

154. The system of claim 153, wherein the one or more speech sounds form a sentence.

155. The system of claim 153 or 154, wherein the subject is limited to a specified word set for the attempted speech.

156. The system of claim 155, wherein the word set comprises words for expressing basic concepts and/or caregiving needs.

157. The system of claim 155 or 156, wherein the word set comprises 100 words or more.

158. The system of claim 157, wherein the word set comprises 350 words or more.

159. The system of claim 158, wherein the word set comprises 1000 words or more.

160. The system of any of claims 155-159, wherein the subject is limited to two or more word sets for the attempted speech.

161. The system of claim 160, wherein the processor is configured to switch between word sets.

162. The system of any of claims 133-161, wherein the decoding machine learning model comprises a neural network.

163. The system of claim 162, wherein the neural network comprises one or more convolutional layers.

164. The system of claim 162 or 163, wherein the neural network is bidirectional.

165. The system of claim 164, wherein the neural network is a recurrent neural network (RNN).

166. The system of claim 165, wherein the RNN comprises gated recurrent units (GRUs) or long-short term memory (LSTM).

167. The system of claim 166, wherein the RNN comprises GRUs.

168. The system of any of claims 133-167, wherein one or more discrete speech units of a set of discrete speech units are decoded from the recorded brain electrical signal data.

169. The system of claim 168, wherein each speech sound is decoded from one or more discrete speech units.

170. The system of any of claims 168-169, wherein discrete speech units are continuously decoded at a uniform frequency.

171. The system of claim 170, wherein the frequency is 200 Hz or more.

172. The system of any of claims 168-171, wherein the set of discrete speech units are generated using an encoding machine learning model.

173. The system of claim 172, wherein the encoding machine learning model comprises a neural network.

174. The system of claim 173, wherein the neural network comprises one or more convolutional layers.

175. The system of claim 173 or 174, wherein the neural network is bidirectional.

176. The system of claim 175, wherein the neural network comprises a transformer encoder.

177. The system of claim 176, wherein the machine learning model is a Hidden-Unit Bidirectional Encoder Representations from Transformers (HuBERT) model.

178. The system of any of claims 172-177, wherein the set of discrete speech units are generated by training the encoding machine learning model.

179. The system of claim 178, wherein the training is self-supervised training.

180. The system of any of claims 172-179, wherein the processor is further programmed to:
obtain reference electronic speech waveforms for a plurality of phrases;
encode each reference speech waveform into a temporal sequence of discrete speech units using the encoding model;
record the brain electrical signal data associated with attempted speech by the subject for each one of the plurality of phrases;
train the decoding machine learning model to predict the most likely discrete speech unit associated with a segment of electrical signal data using the speech units derived from the reference speech waveforms and the brain electrical signal data associated with attempted speech for each one of the plurality of phrases.

181. The system of claim 180, wherein the system further comprises a microphone configured to generate reference speech waveforms from a recruited speaker.

182. The system of claim 180, wherein the processor further comprises a text-to-speech algorithm configured to generate the reference speech waveforms.

183. The system of any of claims 180-182, wherein the decoding machine learning model is trained to learn mappings between neural activity patterns of electrical signals in the brain electrical signal data and the discrete speech units.

184. The system of any of claims 172 to 183, wherein each speech sound is decoded from one or more discrete speech units using a speech synthesizer.

185. The system of claim 184, wherein the speech synthesizer comprises a machine learning model.

186. The system of claim 185, wherein the synthesizing machine learning model comprises a neural network.

187. The system of claim 186, wherein the neural network comprises one or more convolutional layers.

188. The system of claim 186 or 187, wherein the neural network is bidirectional.

189. The system of claim 188, wherein the neural network is an RNN.

190. The system of claim 189, wherein the RNN comprises GRUs or LSTM.

191. The system of claim 190, wherein the RNN comprises one or more LSTM layers.

192. The system of any of claims 186 to 191, wherein the neural network comprises an attention mechanism.

193. The system of any of claims 185 to 192, wherein the synthesizing machine learning model is configured to generate a spectrogram from the one or more discrete speech units.

194. The system of claim 193, wherein the spectrogram is a mel spectrogram.

195. The system of claim 193 or 194, wherein the speech synthesizer further comprises a vocoder configured to synthesize an electronic speech waveform from the spectrogram.

196. The system of claim 195, wherein the vocoder comprises a machine learning model.

197. The system of claim 196, wherein the vocoder machine learning model comprises a neural network.

198. The system of claim 197, wherein the neural network is an RNN.

199. The system of any of claims 195 to 198, wherein the processor is further programmed to transform the electronic speech waveform into a personalized electronic speech waveform.

200. The system of claim 199, wherein the personalized electronic speech waveform resembles speech in the subject's own voice.

201. The system of claim 199 or 200, wherein the transforming is performed using a machine learning model.

202. The system of claim 201, wherein the transforming machine learning model comprises a neural network.

203. The system of claim 202, wherein the neural network is based on transformer architecture.

204. The system of any of claims 195-203, wherein the processor is further programmed to convert the electronic speech waveform into an audible speech waveform.

205. The system of claim 204, wherein the electronic speech waveform is converted into an audible speech waveform using a loudspeaker.

206. The system of any of claims 133-205, wherein the processor is provided by a computer or handheld device.

207. The system of claim 206, wherein the handheld device is a cell phone or a tablet.

208. A kit comprising the system of any of claims 133-207 and instructions for decoding brain electrical signal data associated with attempted speech by a subject.

209. A method of controlling an electronic output device or system to perform one or more actions using brain electrical signals, the method comprising:
positioning a neural recording device comprising an electrode at a location in a sensorimotor cortex region of the brain of the subject to record brain electrical signal data associated with an attempted action by the subject;
positioning an interface in communication with a computing device at a location on the head of the subject, wherein the interface is connected to the neural recording device;
recording the brain electrical signal data associated with the attempted action by the subject using the neural recording device, wherein the interface receives the brain electrical signal data from the neural recording device and transmits the brain electrical signal data to a processor of the computing device;
decoding one or more electronic output device or system actions from the recorded brain electrical signal data, wherein the processor is programmed to use a machine learning model for the decoding; and
controlling the electronic output device or system to perform the one or more decoded electronic output device or system actions.

210. The method of claim 209, wherein the subject has difficulty communicating because of anarthria, a stroke, a traumatic brain injury, a brain tumor, or amyotrophic lateral sclerosis.

211. The method of claim 209 or 210, wherein the subject is paralyzed.

212. The method of any of claims 209-211, wherein the subject is quadriplegic and/or experiences partial or total facial paralysis.

213. The method of any of claims 209-212, wherein the location of the neural recording device is on a surface of the sensorimotor cortex region or within the sensorimotor cortex region.

214. The method of any of claims 209-213, wherein the electrode of the neural recording device is positioned on the pial surface of the sensorimotor cortex.

215. The method of any of claims 209-214, wherein the neural recording device is positioned such that the recording device covers regions associated with speech production and language perception.

216. The method of claim 215, wherein the covered regions include a middle portion of the superior and/or middle temporal gyrus, the precentral gyrus, and/or the postcentral gyrus.

217. The method of any of claims 209-216, wherein the neural recording device is centered on the central sulcus.

218. The method of any of claims 209-217, wherein the neural recording device comprises an electrocorticography (ECoG) electrode array.

219. The method of claim 218, wherein the ECoG electrode array is a high-density array.

220. The method of claim 219, wherein the high-density array comprises 200 electrodes or more.

221. The method of any of claims 218-220, wherein the electrode array comprises non-penetrating surface electrodes.

222. The method of any of claims 209-221, wherein the interface comprises a percutaneous pedestal connector attached to the subject's cranium.

223. The method of any of claims 209-222, wherein the interface further comprises a headstage connected to the percutaneous pedestal connector.

224. The method of claim 223, wherein the headstage processes and digitizes the brain electrical signal data before transmitting the data to the processor.

225. The method of any of claims 209-224, wherein the electrical signal data comprises high-gamma frequency content features.

226. The method of any of claims 209-225, wherein the electrical signal data comprises neural oscillations in a range from 70 Hz to 150 Hz.

227. The method of any of claims 209-226, wherein the electrical signal data comprises low-frequency signals.

228. The method of claim 227, wherein the electrical signal data comprises neural oscillations in a range from 0.3 Hz to 17 Hz.

229. The method of any of claims 209-228, wherein the brain electrical signal data is recorded from a sensorimotor cortex region selected from the precentral gyrus, postcentral gyrus, superior temporal gyrus, middle temporal gyrus, or any combination thereof.

230. The method of any of claims 209-229, wherein the method further comprises mapping the brain of the subject to identify an optimal location for positioning the electrode for recording the brain electrical signals associated with the attempted speech by the subject.

231. The method of any of claims 209-230, wherein the subject is limited to a specified action set for the attempted action.

232. The method of claim 231, wherein the action set comprises actions for expressing basic emotions and/or communicating caregiving needs.

233. The method of claim 231 or 232, wherein the action set comprises 6 actions or more.

234. The method of claim 233, wherein the action set comprises 100 actions or more.

235. The method of claim 234, wherein the action set comprises 500 actions or more.

236. The method of any of claims 231-235, wherein the subject is limited to two or more action sets for the attempted action.

237. The method of claim 28236 wherein the subject may switch between action sets.

238. The method of any of claims 209-237, wherein the decoding machine learning model comprises a neural network.

239. The method of claim 238, wherein the neural network comprises one or more convolutional layers.

240. The method of claim 238 or 239, wherein the neural network is bidirectional.

241. The method of claim 240, wherein the neural network is a recurrent neural network (RNN).

242. The method of claim 241, wherein the RNN comprises gated recurrent units (GRUs) or long-short term memory (LSTM).

243. The method of claim 242, wherein the RNN comprises GRUs.

244. The method of any of claims 209-243, wherein each action of the device or system is discretized into one or more action representations.

245. The method of claim 244, wherein the action representations are decoded from the recorded brain electrical signal data.

246. The method of claim 245, wherein each action of the device or system is decoded from one or more action representations.

247. The method of claim 246, wherein discrete action representations are continuously decoded from the recorded brain electrical signal data at a uniform frequency.

248. The method of claim 247, wherein the frequency is 50 Hz or more.

249. The method of claim 248, wherein the frequency is 200 Hz or more.

250. The method of any of claims 244-249, wherein the discretization is performed using an autoencoder.

251. The method of claim 250, wherein the autoencoder comprises one or more convolutional layers.

252. The method of claim 250 or 251, wherein the autoencoder uses one or more rectified linear unit (ReLU) activations.

253. The method of claim 252, wherein the autoencoder comprises a vector-quantized variational autoencoder (VQ-VAE).

254. The method of any of claims 250-253, wherein the discretization occurs during training of the autoencoder.

255. The method of claim 254, wherein the training is self-supervised training.

256. The method of any of claims 250-255, wherein the attempted action performed by the subject is different than the one or more electronic output device or system actions.

257. The method of claim 256, wherein the attempted action performed by the subject is a hand gesture and the action of the electronic output device or system is powering on or off.

258. The method of any of claims 250-255, wherein the attempted action performed by the subject corresponds to the one or more electronic output device or system actions.

259. The method of claim 258, wherein the electronic output device or system is a prosthetic limb.

260. The method of claim 258, wherein the electronic output device or system comprises a visual display and/or a loudspeaker.

261. The method of claim 260, wherein the visual display and/or loudspeaker is configured to present a humanoid avatar.

262. The method of claim 261, wherein the one or more electronic output device or system actions comprise actions performed by the avatar.

263. The method of claim 262, wherein actions performed by the avatar include speech, orofacial movements for speech, and/or non-speech communicative gestures.

264. The method of claim 263, wherein the speech orofacial movements comprise one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction.

265. The method of claim 263, wherein the non-speech communicative gestures comprise the abduction, adduction, flexion, extension, and/or circumduction of one or more body parts.

266. The method of claim 263 or 265, wherein the non-speech communicative gestures include emotional expressions using facial muscles.

267. The method of claim 266, wherein the emotional expressions comprise happy, sad, and

268. The method of any of claims 262-267, wherein the method further comprises:
obtaining reference avatar animations for a plurality of actions;
encoding each reference animation into a temporal sequence of discrete action representations using the autoencoder;
recording the brain electrical signal data associated with attempted action by the subject for each one of the plurality of actions;
training the decoding machine learning model to predict the most likely discrete action representation associated with a segment of electrical signal data using the action representations derived from the reference avatar animations and the brain electrical signal data associated with attempted action for each one of the plurality of actions.

269. The method of claim 268, wherein the reference avatar animations are obtained from an avatar-animation system.

270. The method of any of claim 268 or 268, wherein the decoding machine learning model is trained to learn mappings between neural activity patterns of electrical signals in the brain electrical signal data and the discrete action representations.

271. The method of any of claims 268-270, wherein the training uses a CTC loss function.

272. The method of any of claims 250-271, wherein each action of the avatar is decoded from one or more action representations using the autoencoder.

273. The method of any of claims 268-272, wherein the plurality of actions includes at least two of speech, orofacial movements for speech, and/or non-speech communicative gestures.

274. The method of claim 273, wherein the decoding machine learning model is trained to discriminate between actions performed by different regions of the body and/or between speech associated actions and non-speech communicative gestures.

275. The method of claim 274, wherein the decoding machine learning model is trained to discriminate between finger-flexions and actions associated with attempted speech.

276. The method of any of claims 268-275, wherein the machine learning model is trained to decode orofacial movements for speech using action representations derived from reference avatar animations of the avatar performing the orofacial movements and brain electrical signal data associated with orofacial movements attempted by the subject.

277. The method of any of claims 268-275, wherein the avatar is controlled to perform orofacial movements based on speech decoded from the recorded brain electrical signal data.

278. The method of claim 277, wherein the avatar is controlled to perform orofacial movements using a speech-to-gesture algorithm.

279. The method of any of claims 268-277, wherein a single decoding machine learning model is trained for speech, orofacial speech movement, and non-speech communicative gesture actions.

280. The method of any of claims 268-277, wherein separate decoding machine learning models are trained for speech associated actions and non-speech communicative gestures.

281. The method of any of claims 268-277 and 280, wherein separate decoding machine learning models are trained for speech and orofacial movements for speech.

282. The method of claim 280 or 281, wherein the avatar is controlled using actions decoded from multiple machine learning models.

283. The method of claim 282, wherein the actions decoded from the multiple machine learning models occur concurrently and the avatar is controlled to perform the actions simultaneously.

284. The method of any of claims 273-283, wherein a decoded non-speech communicative gesture action affects how the avatar performs a decoded speech associated action.

285. The method of claim 284, wherein a decoded emotional expression affects the inflection of decoded speech performed by the avatar.

286. The method of any of claims 262-285, wherein the avatar is used by the subject to communicate with one or more individuals in person.

287. The method of any of claims 262-286, wherein the avatar is used by the subject to communicate with one or more individuals in an interactive virtual environment.

288. The method of any of claims 262-287, wherein the avatar is used by the subject to play a video game.

289. The method of any of claims 262-288, wherein the avatar is used by the subject for therapy.

290. The method of claim 289, wherein the avatar is used by the subject for physical therapy.

291. The method of claim 290, wherein the physical therapy involves regaining mobility of a body part after an injury.

292. The method of any of claims 262-291, wherein actions performed by the avatar control one or more electronic devices in the subject's environment.

293. The method of claim 292, wherein actions performed by the avatar control one or more electronic devices in the same room as the subject.

294. The method of claim 293, wherein actions performed by the avatar control one or smart home appliances.

295. The method of any of claims 262-294, wherein the visual display comprises a computer monitor, a television, and/or a visual projection device.

296. The method of any of claims 262-295, wherein the visual display comprises a virtual reality headset, goggles, or contacts.

297. The method of any of claims 262-295, wherein the visual display comprises an augmented reality headset, goggles, or contacts.

298. The method of any of claims 262-297, wherein the method further comprises generating a virtual environment for the avatar.

299. The method of claim 298, wherein the action performed by the avatar is determined using the virtual environment.

300. The method of any of claims 262-299, wherein previous actions performed by the avatar are used to determine the action performed by the avatar.

301. The method of any of claims 209-300, wherein the processor is provided by a computer, a handheld device, or a headset.

302. The method of claim 301, wherein the handheld device is a cell phone or a tablet.

303. A non-transitory computer-readable medium comprising program instructions that, when executed by a processor, cause the processor to perform the method of any one of claims 209-302.

304. A kit comprising the non-transitory computer-readable medium of claim 303 and instructions for decoding brain electrical signal data associated with attempted speech by a subject.

305. A system for controlling an electronic device or system using brain electrical signals configured to perform the method according to any of claims 209-302.

306. A computer implemented method for controlling an electronic output device or system to perform one or more actions using brain electrical signals, the computer performing steps comprising:
receiving the recorded brain electrical signal data associated with the attempted action by the subject; and
decoding one or more electronic output device or system actions from the recorded brain electrical signal data using a machine learning model.

307. The computer implemented method of claim 306, wherein the decoding machine learning model comprises a neural network.

308. The computer implemented method of claim 307, wherein the neural network comprises one or more convolutional layers.

309. The computer implemented method of claim 307 or 308, wherein the neural network is bidirectional.

310. The computer implemented method of claim 309, wherein the neural network is a recurrent neural network (RNN).

311. The computer implemented method of claim 310, wherein the RNN comprises gated recurrent units (GRUs) or long-short term memory (LSTM).

312. The computer implemented method of claim 311, wherein the RNN comprises GRUs.

313. The computer implemented method of any of claims 306-312, wherein the subject is limited to a specified action set for the attempted action.

314. The computer implemented method of claim 313, wherein the action set comprises actions for expressing basic emotions and/or communicating caregiving needs.

315. The computer implemented method of claim 314, wherein the action set comprises 6 actions or more.

316. The computer implemented method of claim 315, wherein the action set comprises 100 actions or more.

317. The computer implemented method of claim 316, wherein the action set comprises 500 actions or more.

318. The computer implemented method of any of claims 313-317, wherein the subject is limited to two or more action sets for the attempted action.

319. The computer implemented method of claim 318, wherein the subject may switch between action sets.

320. The computer implemented method of any of claims 306-319, wherein each action of the device or system is discretized into one or more action representations.

321. The computer implemented method of claim 320, wherein the action representations are decoded from the recorded brain electrical signal data.

322. The computer implemented method of claim 321, wherein each action of the output device or system is decoded from one or more action representations.

323. The computer implemented method of any of claims 320-322, wherein the discretization is performed using an autoencoder.

324. The computer implemented method of claim 323, wherein the autoencoder comprises one or more convolutional layers.

325. The computer implemented method of claim 323 or 324, wherein the autoencoder uses one or more rectified linear unit (ReLU) activations.

326. The computer implemented method of claim 325, wherein the autoencoder comprises a vector-quantized variational autoencoder (VQ-VAE).

327. The computer implemented method of any of claims 323-326, wherein the discretization occurs during training of the autoencoder.

328. The computer implemented method of claim 327, wherein the training is self-supervised training.

329. The computer implemented method of any of claims 306-328, wherein the electronic output device or system comprises a visual display and/or a loudspeaker.

330. The computer implemented method of claim 329, wherein the visual display and/or loudspeaker is configured to present a humanoid avatar.

331. The computer implemented method of claim 330, wherein the one or more electronic output device or system actions comprise actions performed by the avatar.

332. The computer implemented method of claim 331, wherein actions performed by the avatar include speech, orofacial movements for speech, and/or non-speech communicative gestures.

333. The method of claim 332, wherein the non-speech communicative gestures include emotional expressions using facial muscles.

334. The computer implemented method of any of claims 331-333, wherein the method further comprises:
receiving reference avatar animations for a plurality of actions;
encoding each reference avatar animation into a temporal sequence of discrete action representations using the autoencoder;
receiving brain electrical signal data associated with the attempted action by the subject for each one of the plurality of actions;
training the decoding machine learning model to predict the most likely discrete action representation associated with a segment of electrical signal data using the action representations derived from the reference avatar animations and the brain electrical signal data associated with the attempted action for each one of the plurality of actions.

335. The computer implemented method of claim 334, wherein separate decoding machine learning models are trained for speech associated actions and non-speech communicative gestures.

336. The computer implemented method of claim 334 or 335, wherein the avatar is used by the subject to communicate with one or more individuals in person.

337. The computer implemented method of any of claims 334-336, wherein the avatar is used by the subject to communicate with one or more individuals in an interactive virtual environment.

338. The computer implemented method of claim 337, wherein the method further comprises generating a virtual environment for the avatar.

339. The computer implemented method of claim 338, wherein the action performed by the avatar is determined using the virtual environment.

340. The computer implemented method of any of claims 331-339, wherein previous actions performed by the avatar are used to determine the action performed by the avatar.

341. A non-transitory computer-readable medium comprising program instructions that, when executed by a processor, cause the processor to perform the computer implemented method of any one of claims 306-340.

342. A kit comprising the non-transitory computer-readable medium of claim 341 and instructions for decoding brain electrical signal data associated with attempted speech by a subject.

343. A system for controlling an avatar to perform one or more actions using brain electrical signals, the system comprising:
a neural recording device comprising an electrode adapted for positioning at a location in a sensorimotor cortex region of the brain of the subject to record brain electrical signal data associated with an attempted action by the subject;
a processor programmed to use a machine learning model to decode an avatar animation from the recorded brain electrical signal data;
an interface in communication with a computing device, said interface adapted for positioning at a location on the head of the subject, wherein the interface receives the brain electrical signal data from the neural recording device and transmits the brain electrical signal data to the processor; and
a display component for displaying the avatar action from the recorded brain electrical signal data.

344. The system of claim 343, wherein the subject has difficulty with said speech because of anarthria, a stroke, a traumatic brain injury, a brain tumor, or amyotrophic lateral sclerosis.

345. The system of claim 343 or 344, wherein the neural recording device is adapted for positioning on a surface of the sensorimotor cortex region or within the sensorimotor cortex region.

346. The system of any of claims 343-345, wherein the electrode of the neural recording device is adapted for positioning on the pial surface of the sensorimotor cortex.

347. The system of any of claims 343-345, wherein the neural recording device is adapted to be positioned such that the recording device covers regions associated with speech production and language perception.

348. The system of claim 347, wherein the covered regions include a middle portion of the superior and/or middle temporal gyrus, the precentral gyrus, and/or the postcentral gyrus.

349. The system of any of claims 343-348, wherein the neural recording device is adapted to be centered on the central sulcus.

350. The system of any of claims 343-349, wherein the neural recording device comprises an electrocorticography (ECoG) electrode array.

351. The system of claim 350, wherein the ECoG electrode array is a high-density array.

352. The system of claim 351, wherein the high-density array comprises 200 electrodes or more.

353. The system of any of claims 350-352, wherein the electrode array comprises non-penetrating surface electrodes.

354. The system of any of claims 343-353, wherein the interface comprises a percutaneous pedestal connector attached to the subject's cranium.

355. The system of any of claims 343-354, wherein the interface further comprises a headstage connected to the percutaneous pedestal connector.

356. The system of claim 355, wherein the headstage is configured to process and digitize the brain electrical signal data before transmitting the data to the processor.

357. The system of any of claims 343-356, wherein the electrical signal data comprises high-gamma frequency content features.

358. The system of any of claims 343-357, wherein the electrical signal data comprises neural oscillations in a range from 70 Hz to 150 Hz.

359. The system of any of claims 343-358, wherein the electrical signal data comprises low-frequency signals.

360. The system of claim 359, wherein the electrical signal data comprises neural oscillations in a range from 0.3 Hz to 17 Hz.

361. The system of any of claims 343-360, wherein the brain electrical signal data is recorded from a sensorimotor cortex region selected from the precentral gyrus, postcentral gyrus, superior temporal gyrus, middle temporal gyrus, or any combination thereof.

362. The system of any of claims 343-361, wherein the subject is limited to a specified action set for the attempted action.

363. The system of claim 362, wherein the action set comprises actions for expressing basic emotions and/or communicating caregiving needs.

364. The system of claim 362 or 363, wherein the action set comprises 6 actions or more.

365. The system of claim 364, wherein the action set comprises 100 actions or more.

366. The system of any of claims 362-365, wherein the subject is limited to two or more action sets for the attempted action.

367. The system of claim 366, wherein the processor is configured to switch between action sets.

368. The system of any of claims 343-367, wherein the decoding machine learning model comprises a neural network.

369. The system of claim 368 wherein the neural network comprises one or more convolutional layers.

370. The system of claim 368 or 369, wherein the neural network is bidirectional.

371. The system of claim 370, wherein the neural network is a recurrent neural network (RNN).

372. The system of claim 371, wherein the RNN comprises gated recurrent units (GRUs) or long-short term memory (LSTM).

373. The system of claim 372, wherein the RNN comprises GRUs.

374. The system of any of claims 343-373, wherein the processor is further programmed to discretize each avatar action into one or more action representations.

375. The system of claim 374, wherein the action representations are decoded from the recorded brain electrical signal data.

376. The system of claim 375, wherein each avatar action is decoded from one or more action representations.

377. The system of any of claims 374-376, wherein the discretization is performed using an autoencoder.

378. The system of claim 377, wherein the autoencoder comprises one or more convolutional layers.

379. The system of claim 377 or 378, wherein the autoencoder uses one or more rectified linear unit (ReLU) activations.

380. The system of claim 379, wherein the autoencoder comprises a vector-quantized variational autoencoder (VQ-VAE).

381. The system of any of claims 377-380, wherein the discretization occurs during training of the autoencoder.

382. The system of claim 381, wherein the training is self-supervised training.

383. The system of any of claims 343-382, wherein actions performed by the avatar include speech, orofacial movements for speech, and/or non-speech communicative gestures.

384. The system of claim 383, wherein the speech orofacial movements comprise one or more of: a tongue tip raise, tongue retraction, tongue body raise, tongue advance, lip rounding, pinching nostril flare, upper lip pull, lower lip tuck, lower lip push, lower lip pull, lip flare, jaw opening, lip compression, and/or lip adduction.

385. The system of claim 383, wherein the non-speech communicative gestures comprise the abduction, adduction, flexion, extension, and/or circumduction of one or more body parts.

386. The system of claim 383 or 385, wherein the non-speech communicative gestures include emotional expressions using facial muscles.

387. The system of claim 58, wherein the emotional expressions comprise happy, sad, and/or

388. The system of any of claims 377-387, wherein the processor is further programmed to:
obtain reference avatar animations for a plurality of actions;
encode each reference animation into a temporal sequence of discrete action representations using the autoencoder;
record brain electrical signal data associated with the attempted action by the subject for each one of the plurality of actions;
train the decoding machine learning model to predict the most likely discrete action representation associated with a segment of electrical signal data using the action representations derived from the reference avatar animations and the brain electrical signal data associated with attempted action for each one of the plurality of actions.

389. The system of claim 388, wherein the decoding machine learning model is trained to learn mappings between neural activity patterns of electrical signals in the brain electrical signal data and the discrete action representations.

390. The system of claim 388 or 389, wherein the training uses a CTC loss function.

391. The system of any of claims 377-390, wherein each action of the avatar is decoded from one or more action representations using the autoencoder.

392. The system of any of claims 388-391, wherein the plurality of actions includes at least two of speech, orofacial movements for speech, and/or non-speech communicative gestures.

393. The system of claim 392, wherein the decoding machine learning model is trained to discriminate between actions performed by different regions of the body and/or between speech associated actions and non-speech communicative gestures.

394. The system of any of claims 377-393, wherein separate decoding machine learning models are trained for speech associated actions and non-speech communicative gestures.

395. The system of claim 394, wherein separate decoding machine learning models are trained for speech and orofacial movements for speech.

396. The system of claim 394 or 395, wherein the avatar is controlled using actions decoded from multiple machine learning models.

397. The system of claim 396, wherein the actions decoded from the multiple machine learning models occur concurrently and the avatar is controlled to perform the actions simultaneously.

398. The system of any of claims 343-397, wherein the visual display comprises a computer monitor, a television, and/or a visual projection device.

399. The system of any of claims 343-398, wherein the visual display comprises a virtual reality headset, goggles, or contacts.

400. The system of any of claims 343-399, wherein the visual display comprises an augmented reality headset, goggles, or contacts.

401. The system of any of the claims 343-400, wherein the processor is further programmed to generate a virtual environment for the avatar.

402. The system of claim 401, wherein the action performed by the avatar is determined using the virtual environment.

403. The system of any of the claims 343-402, wherein previous actions performed by the avatar are used to determine the action performed by the avatar.

404. The system of any of claims 343-403, wherein the processor is provided by a computer, a handheld device, or a headset.

405. The system of claim 404, wherein the handheld device is a cell phone or a tablet.

406. A kit comprising the system of any of claims 343-405 and instructions for decoding brain electrical signal data associated with attempted speech by a subject.
